(19) United States
(12) Patent Application Publication
(10) Pub. No.: US 2026/0262442 A1
(43) Pub. Date: Sep. 3, 2026

(54) ARYLAMINE COMPOUND, ORGANIC ELECTROLUMINESCENT DEVICE, AND ELECTRONIC APPARATUS

(71) Applicant: SHAANXI LIGHTE OPTOELECTRONICS MATERIAL CO., LTD., Xi'an, Shaanxi (CN)

(72) Inventor: Fumin YUE, Xi'an, Shaanxi (CN)

(21) Appl. No.: 18/862,100

(22) PCT Filed: Sep. 11, 2023
(86) PCT No.: PCT/CN2023/118114

§ 371 Date: Oct. 31, 2024

(30) Foreign Application Priority Data
May 11, 2023 (CN) 202310531460.6

(51) Int. Cl. H10K 85/60; C07B 59/00; C07D 307/91; C07D 405/14; C07D 407/10; C07D 407/14; C07D 409/10; C07D 409/12; C07D 409/14; H10K 50/15

(52) U.S. Cl. CPC H10K 85/633; C07B 59/002; C07D 307/91; C07D 405/14; C07D 407/10; C07D 407/14; C07D 409/10; C07D 409/12; C07D 409/14; H10K 85/636; H10K 50/156; H10K 85/615; H10K 85/626; H10K 85/6572; H10K 85/6574; H10K 85/6576

(57) ABSTRACT

The present disclosure relates to the technical field of organic electroluminescent materials, and provides an arylamine compound, an organic electroluminescent device comprising same, and an electronic apparatus. In the arylamine compound of the present disclosure, the dibenzofuran (dibenzothiophene) parent nucleus is connected with a dibenzopenta-membered ring at its 1-position, while with an arylamine at its 2-position. Such a configuration ensures the para-conjugation effect between the arylamine and the O or S atoms in the parent nucleus, as well as the ortho-conjugation effect between the arylamine and the dibenzopenta-membered ring at the 1-position, such that this compound has high hole mobility, which is beneficial for improving the luminous efficiency of the device; secondly, due to the introduction of the dibenzopenta-membered ring, the steric hindrance effect increases the steric hindrance between molecules, avoiding the planar stacking of molecules, thereby improving the film forming properties of the material and extending the lifetime of the device.

### CROSS-REFERENCE TO RELATED DISCLOSURES

This present disclosure claims the priority of Chinese patent application No. 202310531460.6 filed on May 11, 2023, which is incorporated herein by reference in its entirety as a part of this disclosure.

### FIELD OF THE INVENTION

The present disclosure relates to the technical field of organic electroluminescent materials, and in particular to an arylamine compound, and an organic electroluminescent device comprising same and an electronic apparatus.

### BACKGROUND OF THE INVENTION

With the development of electronic technology and the progress of material science, the application range of electronic components used to realize electroluminescence or photoelectric conversion is being more and more extensive. An organic electroluminescent device (OLED) usually comprises: a cathode and an anode disposed opposite to each other, and a functional layer disposed between the cathode and the anode. The functional layer is composed of multiple organic or inorganic film layers, and generally comprises an organic light-emitting layer, a hole transport layer, an electron transport layer, and so on. When a voltage is applied to the anode and the cathode, an electric field is generated between the two electrodese. Under the influence of the electric field, the electrons on the cathode side move to the electroluminescent layer, and the holes on the anode side also move to the luminescent layer. Electrons and holes combine in the electroluminescent layer to form excitons, which are in an excited state and release energy outward, thereby causing the electroluminescent layer to emit light externally.

In existing organic electroluminescent devices, the main problems are reflected in lifetime and efficiency. As the display area increases, the driving voltage also increases. Research on improving the performance of OLED light-emitting devices includes: reducing the driving voltage of the device, improving the luminous efficiency of the device, extending the service life of the device and so on. To improve the performance of OLED devices, a multi-layer sandwich structure is often used in the design of device structures, i.e., an anode, a cathode, and an organic functional layer together form a complete device. Hole-transporting materials are materials that can accept and effectively transport positively charged hole carriers or block the transport of electrons. They typically have high hole mobility and low ionization potential and are a crucial component in an organic electroluminescent device. It is necessary to continue to develop a new hole transport material to further improve the performance of organic electroluminescent devices.

### SUMMARY OF THE INVENTION

Directed against the above problems in the prior art, an objective of the present disclosure is to provide an arylamine compound, and an organic electroluminescent device and an electronic apparatus using the same. The arylamine compound, when used in an organic electroluminescent device, can improve the performance of the device.

According to a first aspect of the present disclosure, there is provided an arylamine compound having a structure shown in Formula 1:
the arylamine compound has a structure shown in Formula 1:

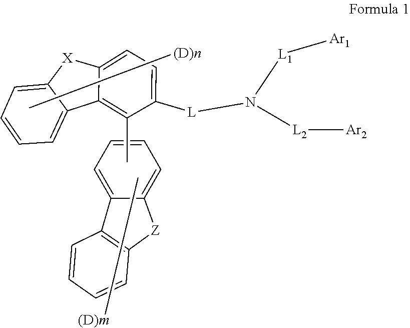

wherein X is selected from O and S; Z is selected from O, S, and C(R₁R₂);
R₁ and R₂ are the same or different, and are each independently selected from an alkyl having 1 to 4 carbon atoms and a deuteroalkyl having 1 to 4 carbon atoms;
m represents the number of D, and m is selected from 0, 1, 2, 3, 4, 5, 6, and 7;
n represents the number of D, and n is selected from 0, 1, 2, 3, 4, 5, and 6;
L is selected from a single bond and a substituted or unsubstituted phenylene;
L₁ and L₂ are the same or different, and are each independently selected from the group consisting of a single bond, a substituted or unsubstituted arylene having 6 to 15 carbon atoms, and a substituted or unsubstituted heteroarylene having 12 to 18 carbon atoms;
the substituent(s) in L, L₁, and L₂ are the same or different, and are each independently selected from the group consisting of a deuterium, a cyano, a halogen group, an alkyl having 1 to 4 carbon atoms, a haloalkyl having 1 to 4 carbon atoms, a deuteroalkyl having 1 to 4 carbon atoms, and a phenyl;
Ar₁ and Ar₂ are the same or different, and are each independently selected from a substituted or unsubstituted aryl having 6 to 40 carbon atoms and a substituted or unsubstituted heteroaryl having 5 to 40 carbon atoms;
the substituent(s) in Ar₁ and Ar₂ are the same or different, and are each independently selected from the group consisting of a deuterium, a cyano, a halogen group, an alkyl having 1 to 10 carbon atoms, a haloalkyl having 1 to 10 carbon atoms, a deuteroalkyl having 1 to 10 carbon atoms, an alkoxy having 1 to 10 carbon atoms, a trialkylsilyl having 3 to 12 carbon atoms, a triphenylsilyl, an aryl having 6 to 20 carbon atoms, a deuteroaryl having 6 to 20 carbon atoms, a heteroaryl having 3 to 20 carbon atoms, a cycloalkyl having 3 to 10 carbon atoms, an alkylthio having 1 to 10 carbon atoms, an aryloxy having 6 to 20 carbon atoms, and an arylthio having 6 to 20 carbon atoms; optionally, any two adjacent substituents form a 5- to 15-membered ring.

According to a second aspect of the present disclosure, there is provided an organic electroluminescent device, comprising an anode and a cathode disposed opposite to each other, and a functional layer disposed between the anode and the cathode; the functional layer comprises the aforementioned arylamine compound.

According to a third aspect of the present disclosure, there is provided an electronic apparatus, comprising the organic electroluminescent device of the second aspect.

In the arylamine compound of the present disclosure, the dibenzofuran (dibenzothiophene) parent nucleus is connected with a dibenzopenta-membered ring at its 1-position, while with an arylamine at its 2-position. Such a configuration ensures a para-conjugation effect between the arylamine and the O or S atom in the parent nucleus, as well as the ortho-conjugation effect between the arylamine and the dibenzopenta-membered ring at the 1-position, such that this compound has high hole mobility, which is beneficial for improving the luminous efficiency of the device; secondly, due to the introduction of the dibenzopenta-membered ring, the steric hindrance effect increases the stereo-structure between molecules, which avoids the planar stacking of molecules, thereby improving the film forming properties of the material and extending the lifetime of a device.

### BRIEF DESCRIPTION OF THE DRAWINGS

The drawings are provided for a further understanding of the present disclosure and constitute a part of the specification, and are used to explain the present disclosure together with the following specific embodiments, but do not constitute a limitation of the present disclosure.

FIG. 1 is a schematic structural diagram of an organic electroluminescent device according to an embodiment of the present disclosure.

FIG. 2 is a schematic structural diagram of an electronic apparatus according to an embodiment of the present disclosure.

List of Reference Signs

| | |
|---|---|
| 100: Anode; | 200: Cathode; |
| 300: Functional layer; | 310: Hole injection layer; |
| 321: Hole transport layer; | 322: Hole auxiliary layer; |
| 330: Organic light-emitting layer; | 340: Hole blocking layer; |
| 350: Electron transport layer; | 360: Electron injection layer; |
| 320: Hole transport area; | 400: Electronic apparatus. |

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein. On the contrary, these embodiments are provided such that the present disclosure is more comprehensive and complete, and the concepts of these exemplary embodiments can be conveyed fully to those skilled in the art. Features, structures, or characteristics described herein can be combined in one or more embodiments in any suitable manner. In the following description, various specific details are provided to give a full understanding of the embodiments of the present disclosure.

In a first aspect, the present disclosure provides an arylamine compound having a structure shown in Formula 1:

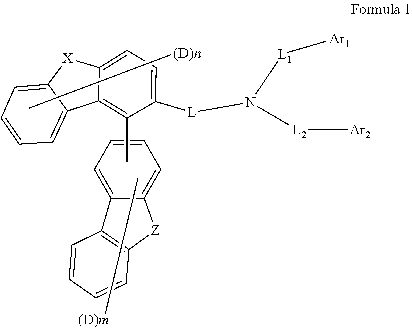

wherein X is selected from O and S; Z is selected from O, S, and C(R₁R₂);
R₁ and R₂ are the same or different, and are each independently selected from an alkyl having 1 to 4 carbon atoms and a deuteroalkyl having 1 to 4 carbon atoms;
m represents the number of D, and m is selected from 0, 1, 2, 3, 4, 5, 6, and 7;
n represents the number of D, and n is selected from 0, 1, 2, 3, 4, 5, and 6;
L is selected from a single bond and a substituted or unsubstituted phenylene;
L₁ and L₂ are the same or different, and are each independently selected from the group consisting of a single bond, a substituted or unsubstituted arylene having 6 to 15 carbon atoms, and a substituted or unsubstituted heteroarylene having 12 to 18 carbon atoms;
the substituent(s) in L, L₁, and L₂ are the same or different, and are each independently selected from the group consisting of a deuterium, a cyano, a halogen group, an alkyl having 1 to 4 carbon atoms, a haloalkyl having 1 to 4 carbon atoms, a deuteroalkyl having 1 to 4 carbon atoms, and a phenyl;
Ar₁ and Ar₂ are the same or different, and are each independently selected from a substituted or unsubstituted aryl having 6 to 40 carbon atoms and a substituted or unsubstituted heteroaryl having 5 to 40 carbon atoms;
the substituent(s) in Ar₁ and Ar₂ are the same or different, and are each independently selected from the group consisting of a deuterium, a cyano, a halogen group, an alkyl having 1 to 10 carbon atoms, a haloalkyl having 1 to 10 carbon atoms, a deuteroalkyl having 1 to 10 carbon atoms, an alkoxy having 1 to 10 carbon atoms, a trialkylsilyl having 3 to 12 carbon atoms, a triphenylsilyl, an aryl having 6 to 20 carbon atoms, a deuteroaryl having 6 to 20 carbon atoms, a heteroaryl having 3 to 20 carbon atoms, a cycloalkyl having 3 to 10 carbon atoms, an alkylthio having 1 to 10 carbon atoms, an aryloxy having 6 to 20 carbon atoms, and an arylthio having 6 to 20 carbon atoms; optionally, any two adjacent substituents form a 5- to 15-membered ring.

In the present disclosure, the terms “optional” and “optionally” mean that the subsequently described event or circumstance may or may not occur. For example, “optionally, any two adjacent substituents in Ar₁ and Ar₂ form a 5- to 15-membered ring” includes both scenarios where any two adjacent substituents form a ring and where any two adjacent substituents exist independently and do not form a ring. “Any two adjacent substituents” can include two substituents on the same atom and can also include two substituents on two adjacent atoms, respectively; wherein, when there are two substituents on the same atom, the two substituents can form a saturated or unsaturated spiro-ring with the atom to which they are connected together; when each of two adjacent atoms has one substituent, the two substituents can be condensed into a ring.

In the present disclosure, a ring system that is formed of n atoms is a n-membered ring. For example, a phenyl is a 6-membered ring. 5- to 15-membered ring refers to a cyclic group having 5 to 15 ring atoms, and the ring atoms can be carbon atoms or heteroatoms selected from O, S, and N, etc. 3- to 15-membered ring is, such as, cyclopentane, cyclohexane, fluorene ring, and benzene ring, etc.

In the present disclosure, the descriptive expression “ . . . are each independently” may be used interchangeably with the descriptive expressions “ . . . are respectively and independently” and “each . . . is independently”, and all these expressions should be interpreted in a broad sense. They can not only mean that, for the same symbol in different groups, specific options expressed by the same symbols are mutual non-influential, but also mean that for the same symbol in the same group, specific options expressed by the same symbols are mutual non-influential. For example,

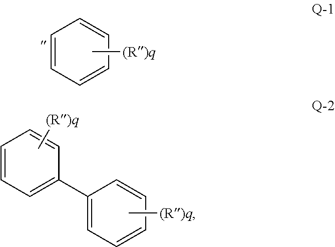

in which each q is independently 0, 1, 2, or 3, and each R″ is independently selected from a hydrogen, a deuterium, a fluoro, and a chloro″, means that the Formula Q-1 represents that there are q substituents R″ on a benzene ring, and each R″ can be the same or different, with mutual non-influence between the options for each R″; Formula Q-2 represents that there are q substituents R″ on each benzene ring of biphenyl, and the number of R″ substituents on the two benzene rings can be the same or different, with mutual non-influence between the options for each R″.

In the present disclosure, such a term “substituted or unsubstituted” means that the functional group defined by the term may or may not have a substituent (hereinafter referred to as Rc for ease of description). For example, “a substituted or unsubstituted aryl” refers to an aryl having substituent Rc or an aryl not having a substituent. The substituent Rc mentioned above may be, such as, a deuterium, a fluoro, a cyano, a heteroaryl, an aryl, a deuteroaryl, a trialkylsilyl, an alkyl, a haloalkyl, a deuteroalkyl, and a cycloalkyl, etc. The number of substituents may be one or more.

In the present disclosure, “more” refers to two or more, such as 1, 2, 3, 4, 5, and 6, etc.

In the present disclosure, the number of carbon atoms of a substituted or unsubstituted functional group refers to the total number of all carbon atoms on this functional group and all substituents of this functional group. For example, if L₁ is a substituted arylene having 12 carbon atoms, the number of all carbon atoms of the arylene and the substituents thereon is 12.

The hydrogen atom in the structure of the compounds in the present disclosure includes various isotope atoms of hydrogen, such as hydrogen (H), deuterium (D), or tritium (T).

The “D” in the structural Formulae of the compounds in the present disclosure represents a deuterium substitution.

In the present disclosure, an aryl refers to an optional functional group or a substituent derived from an aromatic carbon ring. An aryl may be a monocyclic aryl (e.g., phenyl) or a polycyclic aryl. In other words, an aryl may be a monocyclic aryl, a fused cycloaryl, two or more monocyclic aryls linked by carbon-carbon bond conjugation, a monocyclic aryl and a fused cycloaryl linked by carbon-carbon bond conjugation, or two or more fused cycloaryls linked by carbon-carbon bond conjugation. That is, unless otherwise specified, two or more aromatic groups linked by carbon-carbon bond conjugation may also be regarded as an aryl in the present disclosure. Among them, a fused cycloaryl may include, such as, a bicyclic fused aryl (e.g., a naphthyl), a tricyclic fused aryl (e.g., a phenanthryl, a fluorenyl, and an anthryl), etc. An aryl does not contain a heteroatom such as B, N, O, S, P, Se, and Si. Examples of the aryl include, but are not limited to, a phenyl, a naphthyl, a fluorenyl, a spirobifluorenyl

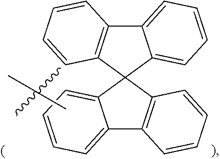

an anthracenyl, a phenanthryl, a biphenyl, a terphenyl, a tetraphenyl, a pentaphenyl, a triphenylene

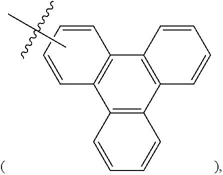

perylenyl, a benzo[9,10]phenanthryl, a pyrenyl, a benzofluoranthryl, and a chrysenyl, ect.

In the present disclosure, “an arylene” involved refers to a divalent group formed by further removing one or more hydrogen atom(s) from an aryl.

In the present disclosure, a terphenyl includes

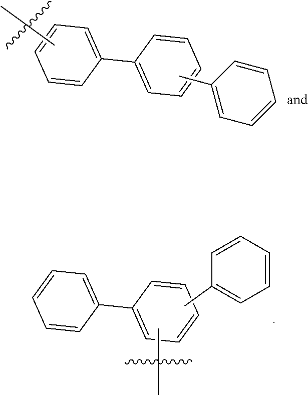

In the present disclosure, the number of carbon atoms of a substituted or unsubstituted aryl (arylene) may be 6, 8, 10, 12, 13, 14, 15, 16, 17, 18, 19, 20, 21, 22, 23, 24, 25, 30, 31, 32, 33, 34, 35, 36, 37, 38, 39, or 40. In some embodiments, a substituted or unsubstituted aryl is a substituted or unsubstituted aryl having 6 to 30 carbon atoms; in some other embodiments, a substituted or unsubstituted aryl is a substituted or unsubstituted aryl having 6 to 25 carbon atoms; in some other embodiments, a substituted or unsubstituted aryl is a substituted or unsubstituted aryl having 6 to 18 carbon atoms; in some other embodiments, a substituted or unsubstituted aryl is a substituted or unsubstituted aryl having 6 to 15 carbon atoms.

In the present disclosure, a fluorenyl may be substituted by one or more substituent(s). In the case that the above-mentioned fluorenyl is substituted, the substituted fluorenyl may be:

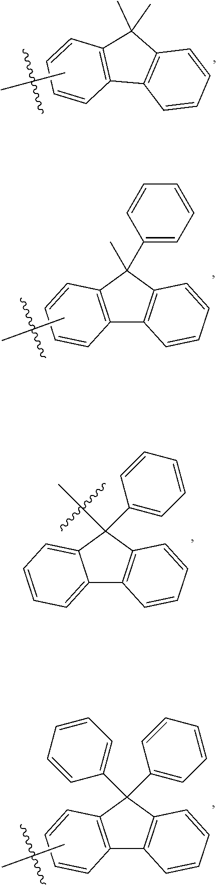

etc, but are not limited thereo.

In the present disclosure, the aryl as a substituent for Ar₁ and Ar₂ is such as, but not limited to, a phenyl, a naphthyl, a phenanthryl, a biphenyl, a fluorenyl, and a dimethylfluorenyl, etc.

In the present disclosure, “a heteroaryl” refers to a monovalent aromatic ring containing 1, 2, 3, 4, 5, or 6 heteroatoms or the derivative thereof. The heteroatoms may be one or more of B, O, N, P, Si, Se, and S. A heteroaryl may be a monocyclic heteroaryl or a polycyclic heteroaryl. In other words, a heteroaryl may be a single aromatic ring system, and may also be multiple aromatic ring systems linked by carbon-carbon bond conjugation, with any one of the aromatic ring systems being an aromatic monocyclic ring or a fused aromatic ring. For example, the heteroaryl may include a thienyl, a furyl, a pyrrolyl, an imidazolyl, a thiazolyl, an oxazolyl, an oxadiazolyl, a triazolyl, a pyridyl, a bipyridyl, a pyrimidinyl, a triazinyl, an acridinyl, a pyridazinyl, a pyrazinyl, a quinolyl, a quinazolinyl, a quinoxalinyl, a phenoxazinyl, a phthalazinyl, a pyridopyrimidinyl, a pyridopyrazinyl, a pyrazinopyrazinyl, an isoquinolyl, an indolyl, a carbazolyl, a benzoxazolyl, a benzimidazolyl, a benzothiazolyl, a benzocarbazolyl, a benzothienyl, a dibenzothienyl, a thienothienyl, a benzofuranyl, a phenanthrolinyl, an isoxazolyl, a thiadiazolyl, a phenothiazinyl, a silafluorenyl, a dibenzofuranyl, and a N-phenylcarbazolyl, a N-pyridylcarbazolyl, a N-methylcarbazolyl, etc, but are not limited to thereto.

In the present disclosure, a heteroarylene involved is a divalent or multivalent group formed by further removing one or more hydrogen atom(s) from a heteroaryl group.

In the present disclosure, the number of carbon atoms of a substituted or unsubstituted heteroaryl (heteroarylene) may be selected from 3, 4, 5, 6, 7, 8, 9, 10, 11, 12, 13, 14, 15, 16, 17, 18, 20, 21, 22, 23, 24, 25, 26, 27, 28, 29, 30, 31, 32, 33, 34, 35, 36, 37, 38, 39, or 40. In some embodiments, a substituted or unsubstituted heteroaryl is a substituted or unsubstituted heteroaryl having 12 to 18 carbon atoms. In some other embodiments, a substituted or unsubstituted heteroaryl is a substituted or unsubstituted heteroaryl having 5 to 12 carbon atoms.

In the present disclosure, the heteroaryl as a substituent for Ar₁ and Ar₂ is, such as, but is not limited to, a pyridyl, a carbazolyl, a dibenzothienyl, a dibenzofuranyl, a benzoxazolyl, a benzothiazolyl, and benzimidazolyl.

In the present disclosure, a substituted heteroaryl may be a heteroaryl in which one or more hydrogen atom(s) are replaced by a group such as a deuterium atom, a halogen group, a cyano, an aryl, a heteroaryl, a trialkylsilyl, an alkyl, a deuteroalkyl, a cycloalkyl, or a haloalkyl etc.

In the present disclosure, an alkyl having 1 to 10 carbon atoms may include a straight-chain alkyl having 1 to 10 carbon atoms or a branched-chain alkyl having 3 to 10 carbon atoms. The number of the carbon atoms of an alkyl may be, for example, 1, 2, 3, 4, 5, 6, 7, 8, 9, or 10. Specific examples of an alkyl include, but are not limited to, a methyl, an ethyl, a n-propyl, an isopropyl, a n-butyl, an isobutyl, a tert-butyl, a n-pentyl, an isopentyl, a neopentyl, and a n-hexyl, etc.

In the present disclosure, a halogen group may be, for example, a fluoro, a chloro, a bromo, and an iodo.

In the present disclosure, specific examples of a trialkylsilyl include, but are not limited to, a trimethylsilyl, a triethylsilyl, etc.

In the present disclosure, a haloalkyl refers to an alkyl substituted with a halogen. The specific examples of a haloalkyl include, but are not limited to, a trifluoromethyl.

In the present disclosure, a deuteroalkyl refers to an alkyl having one or more deuterium substitution(s). Specific examples of a deuteroalkyl include, but are not limited to, a trideuteromethyl.

In the present disclosure, the number of carbon atoms of a cycloalkyl having 3 to 10 carbon atoms may be, for example, 3, 4, 5, 6, 7, 8 or 10. Specific examples of a cycloalkyl include, but are not limited to, a cyclopentyl, a cyclohexyl, and an adamantyl.

In the present disclosure, the number of carbon atoms of a deuteroalkyl having 1 to 10 carbon atoms may be, for example 1, 2, 3, 4, 5, 6, 7, 8 or 10. Specific examples of a deuteroalkyl include, but are not limited to, a trideuteromethyl.

In the present disclosure, the number of carbon atoms of a haloalkyl having 1 to 10 carbon atoms may be, for example 1, 2, 3, 4, 5, 6, 7, 8 or 10. Specific examples of a haloalkyl include, but are not limited to, a trifluoromethyl.

In the present disclosure,

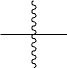

refers to a chemical bond that connects with other groups.

In the present disclosure, a non-positional connection bond refers to a single bond

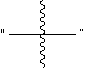

extending from the ring system involved in the non-positional connection bond, which represents that one end of the connection bond can connect to any position in the ring system through which the bond passes, and the other end connects to the rest of the compound molecule. For example, as shown in Formula (f) below, the naphthyl represented by Formula (f) is connected to other positions of the molecule through two non-positional bonds passing through the two rings, which indicates any of possible connection forms shown in Formulae (f-1) to (f-10):

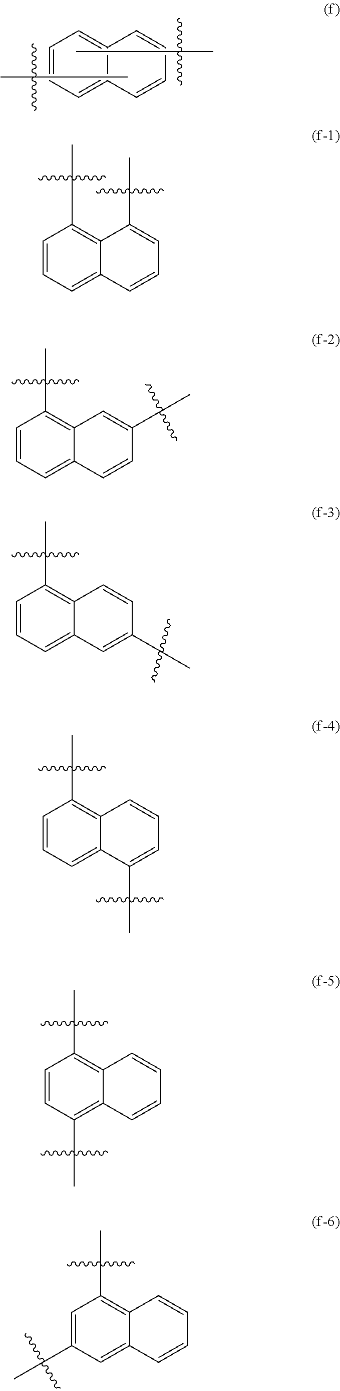

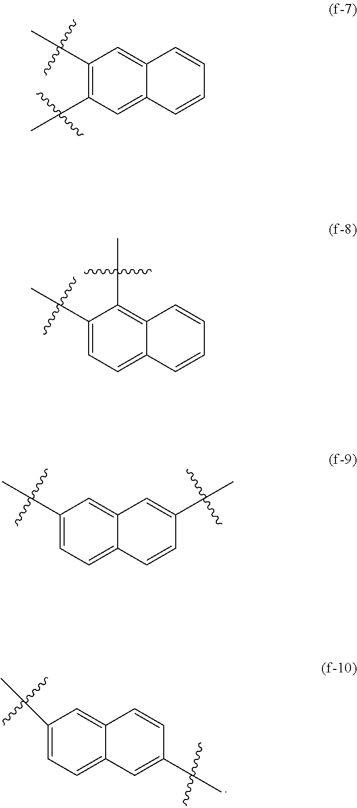 [SMILES: *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, the dibenzofuranyl represented by Formula (X′) is connected to other positions of the molecule via a non-positional connection bond extending from the center of a benzene ring at one side, which indicates any of possible connection form shown in Formulae (X′-1) to (X′-4):

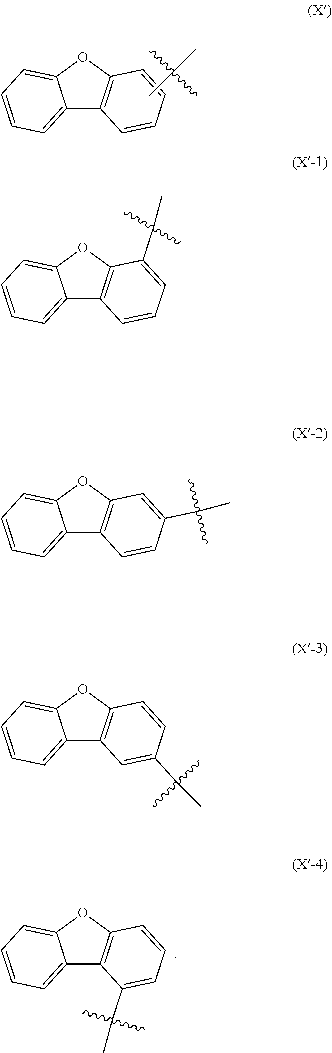

A non-positional substituent in the present disclosure refers to a substituent connected via a single bond extending from the center of a ring system, and it means that the substituent may be connected to any possible position in the ring system. For example, as shown in Formula (Y) below, the substituent R′ represented by Formula (Y) is connected to a quinoline ring via a non-positional connection bond, which indicates any of possible connecting mode shown in Formulae (Y-1) to (Y-7):

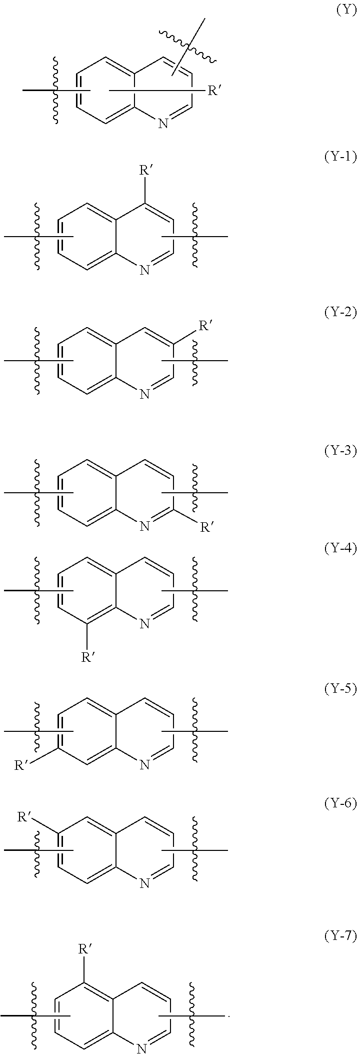

In some embodiments, L₁ and L₂ are each independently selected from a single bond, a substituted or unsubstituted arylene having 6, 7, 8, 9, 10, 11, 12, 13, 14, or 15 carbon atoms, and a substituted or unsubstituted heteroarylene having 12, 13, 14, 15, 16, 17, or 18 carbon atoms.

In some embodiments, L₁ and L₂ are the same or different, and are each independently selected from the group consisting of a single bond, a substituted or unsubstituted phenylene, a substituted or unsubstituted naphthylene, a substituted or unsubstituted biphenylene, a substituted or unsubstituted phenanthrylene, a substituted or unsubstituted dibenzothienylene, a substituted or unsubstituted dibenzofuranylene, and a substituted or unsubstituted carbazolylene.

Optionally, the substituent(s) in L₁ and L₂ are the same or different, and are each independently selected from the group consisting of a deuterium, a fluoro, a cyano, a methyl, an ethyl, an isopropyl, a tert-butyl, a trifluoromethyl, a trideuteromethyl, and a phenyl.

In some embodiments, L₁ and L₂ are each independently selected from a single bond and the group consisting of the following groups:

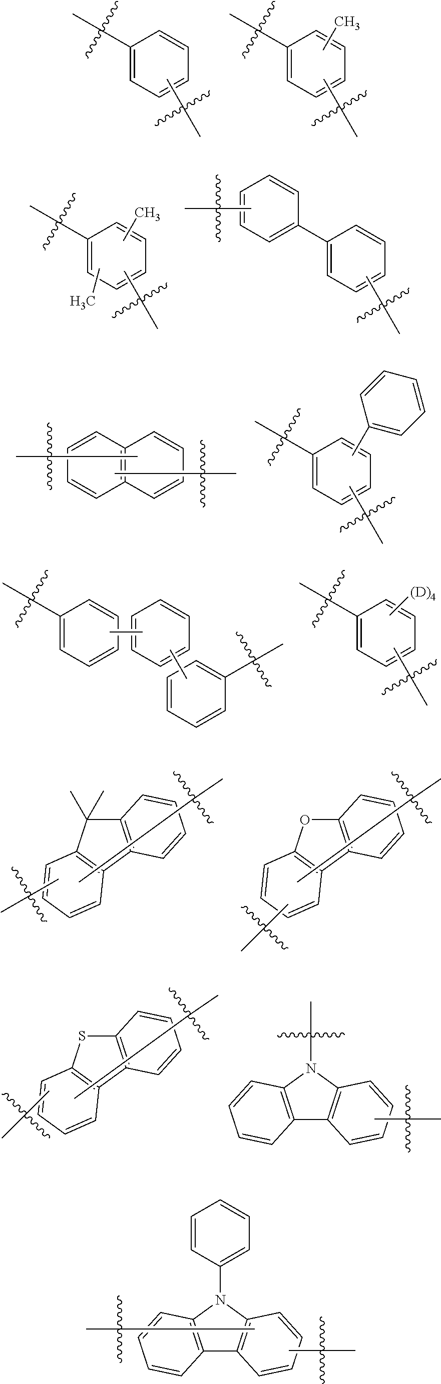

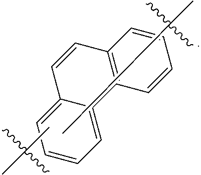

In some embodiments, L₁ and L₂ are each independently selected from a single bond and the group consisting of the following groups:

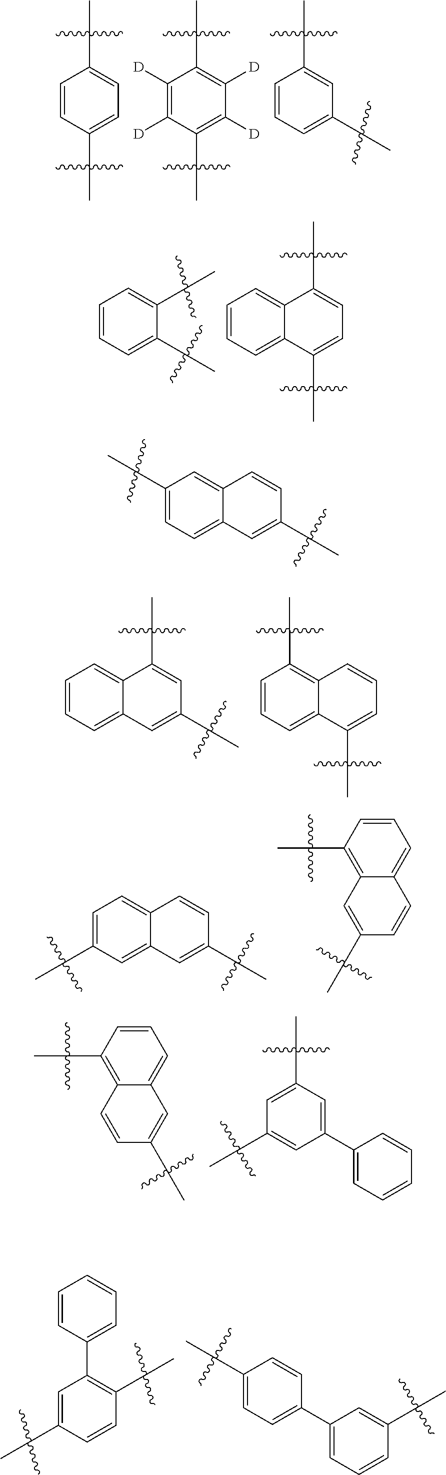

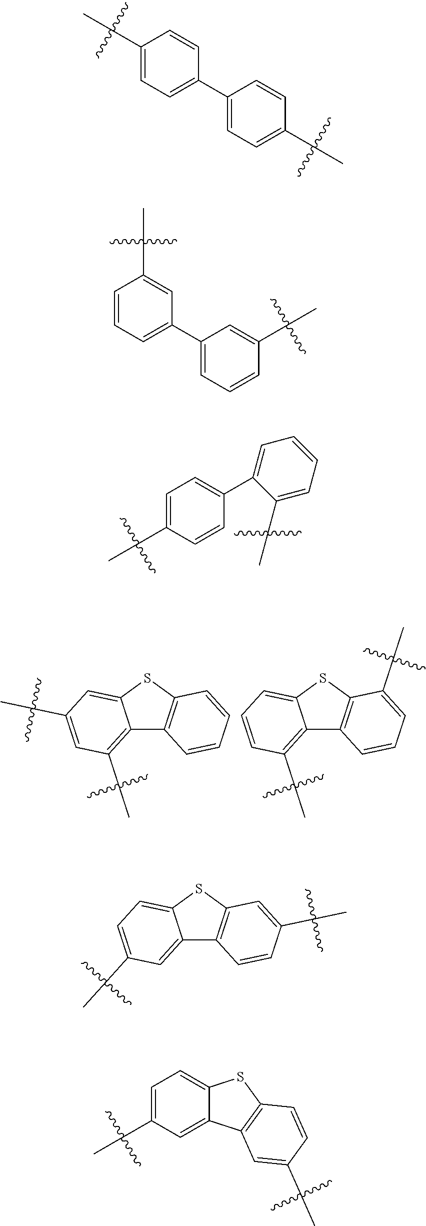

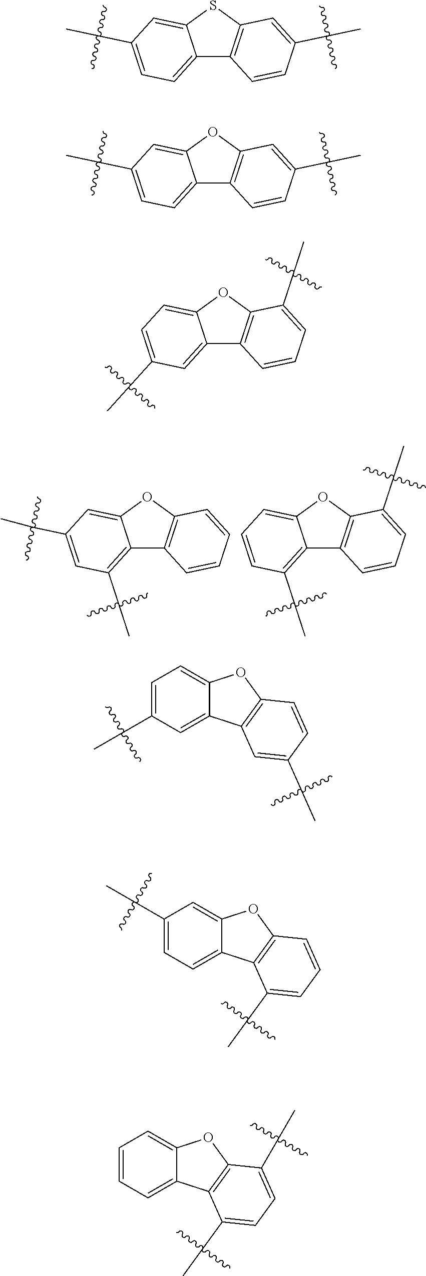

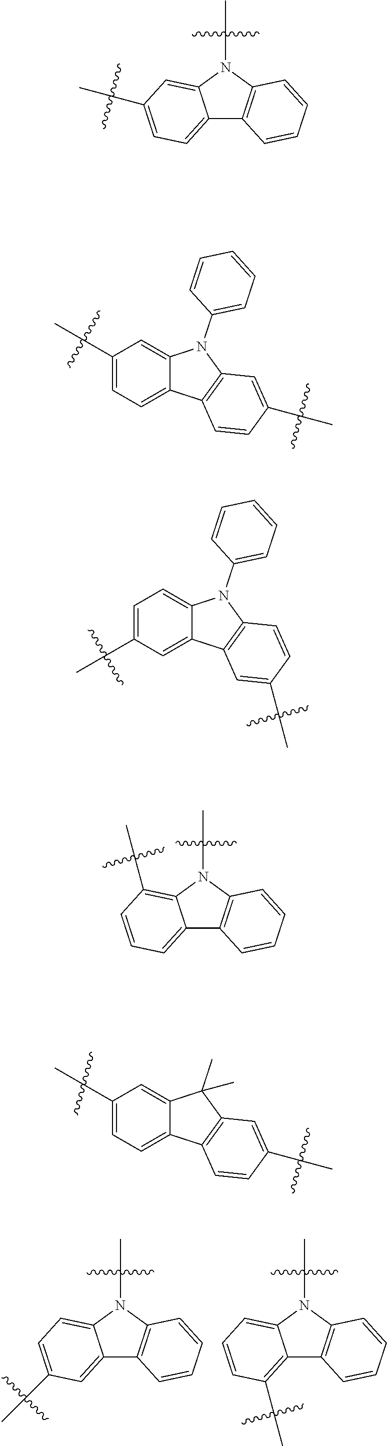

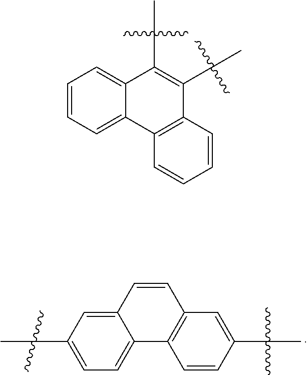 [SMILES: *c1c(*)c2ccccc2c2ccccc12 ; *c1ccc2c(ccc3cc(*)ccc32)c1]

In some embodiments, L is selected from a single bond and the following groups:

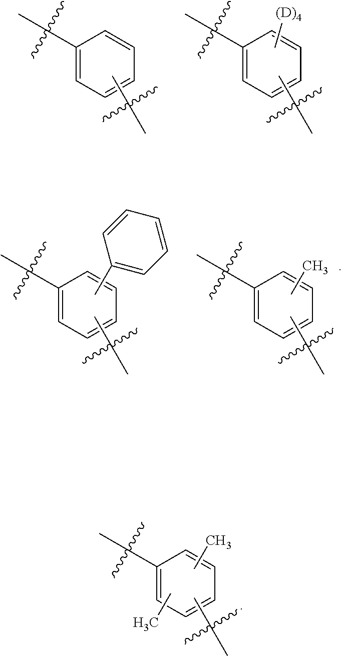

In some embodiments, L is selected from a single bond and the following groups:

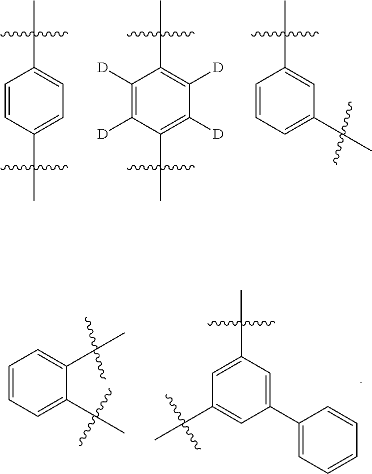

In some embodiments, Ar₁ and Ar₂ are each independently selected from a substituted or unsubstituted aryl having 6, 7, 8, 9, 10, 11, 12, 13, 14, 15, 16, 17, 18, 19, 20, 21, 22, 23, 24, 25, 26, 27, 28, 29, or 30 carbon atoms and a substituted or unsubstituted heteroaryl having 3, 4, 5, 6, 7, 8, 9, 10, 12, 13, 14, 15, 16, 17, 18, 19, 20, 21, 22, 23, 24, 25, 26, 27, 28, 29, or 30 carbon atoms.

Optionally, the substituent(s) in Ar₁ and Ar₂ are each independently selected from the group consisting of a deuterium, a fluoro, a cyano, a deuteroalkyl having 1 to 4 carbon atoms, a trimethylsilyl, a fluoroalkyl having 1 to 4 carbon atoms, a cycloalkyl having 5 to 10 carbon atoms, an alkyl having 1 to 4 carbon atoms, a phenyl, a naphthyl, a pyridyl, a dibenzofuranyl, a dibenzothienyl, and a carbazolyl. Optionally, any two adjacent substituents in Ar₁ and Ar₂ form a benzene ring or a fluorene ring.

In some embodiments, Ar₁ and Ar₂ are each independently selected from the group consisting of a substituted or unsubstituted phenyl, a substituted or unsubstituted biphenyl, a substituted or unsubstituted terphenyl, a substituted or unsubstituted naphthyl, a substituted or unsubstituted anthryl, a substituted or unsubstituted phenanthryl, a substituted or unsubstituted fluorenyl, a substituted or unsubstituted spirodifluorenyl, a substituted or unsubstituted triphenylene, a substituted or unsubstituted pyrenyl, a substituted or unsubstituted perylenyl, a substituted or unsubstituted dibenzothienyl, a substituted or unsubstituted dibenzofuranyl, a substituted or unsubstituted carbazolyl, a substituted or unsubstituted benzothiazolyl, and a substituted or unsubstituted benzoxazolyl.

Optionally, the substituent(s) in Ar₁ and Ar₂ are each independently selected from the group consisting of a deuterium, a fluoro, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a pyridyl, a dibenzofuranyl, a dibenzothienyl, and a carbazolyl; optionally, any two adjacent substituents in Ar₁ and Ar₂ form a benzene ring or a fluorene ring.

In some embodiments, Ar₁ and Ar₂ are each independently selected from a substituted or unsubstituted group W; among them, the unsubstituted group W is selected from the group consisting of the following groups:

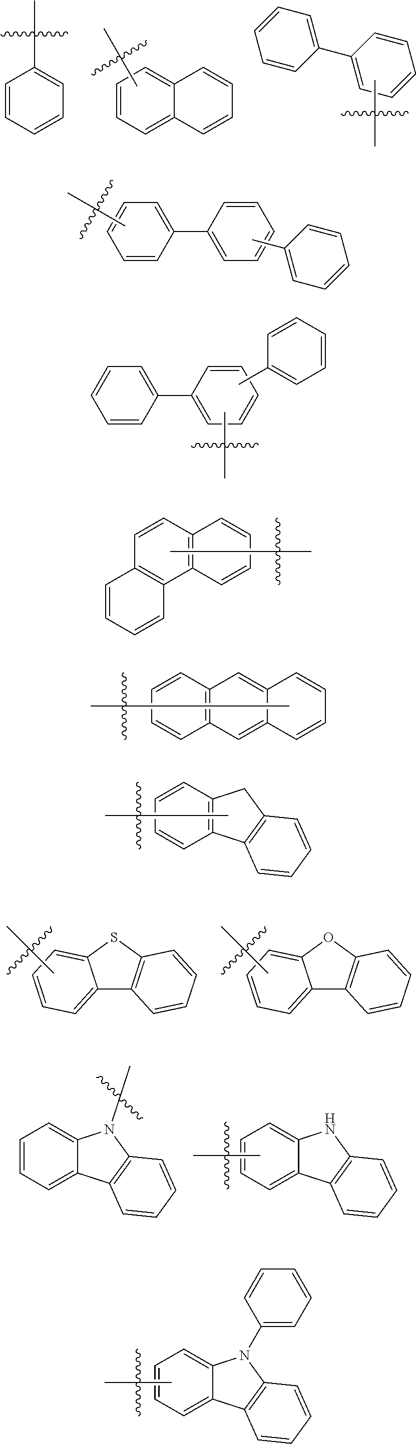

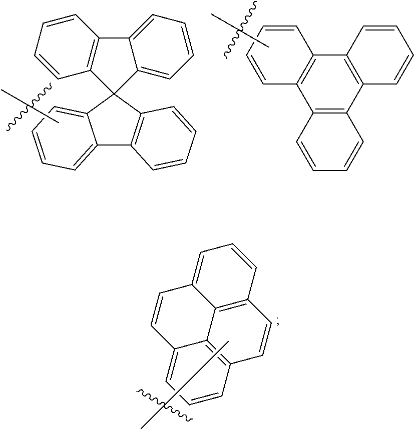

the substituted group W has one or more substituent(s), and the substituent(s) in the substituted group W are each independently selected from the group consisting of a deuterium, a fluoro, a cyano, a trideuteromethyl, a trimethylsilyl, a trifluoromethyl, a cyclopentyl, a cyclohexyl, an adamantyl, a methyl, a ethyl, an isopropyl, a tert-butyl, a phenyl, a naphthyl, a pyridyl, a dibenzofuranyl, a dibenzothienyl, and a carbazolyl, and when the number of the substituents in the group W is greater than 1, each substituent is the same or different.

In some embodiments, Ar₁ and Ar₂ are each independently selected from the group consisting of the following groups:

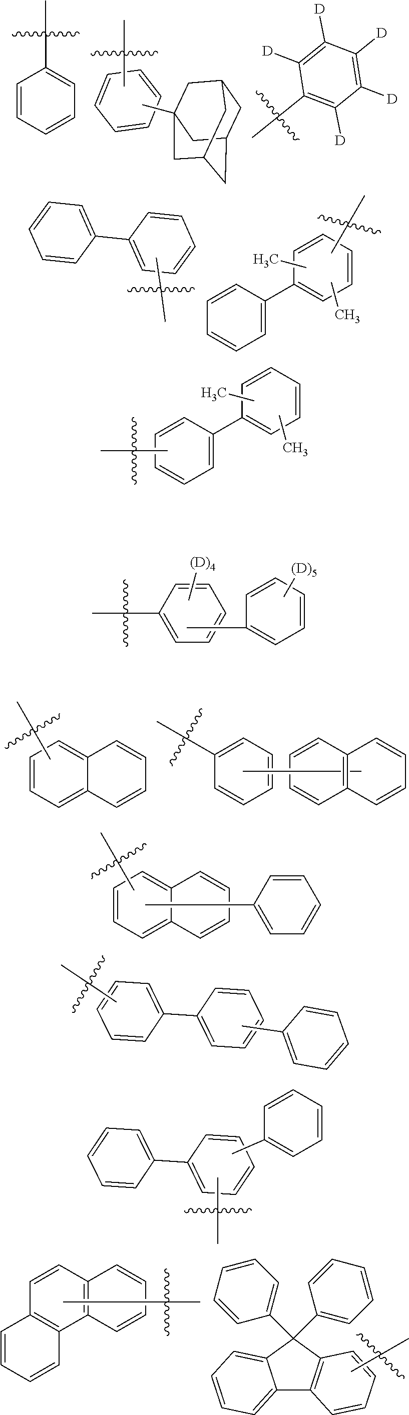

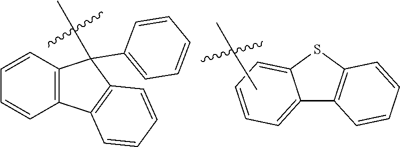

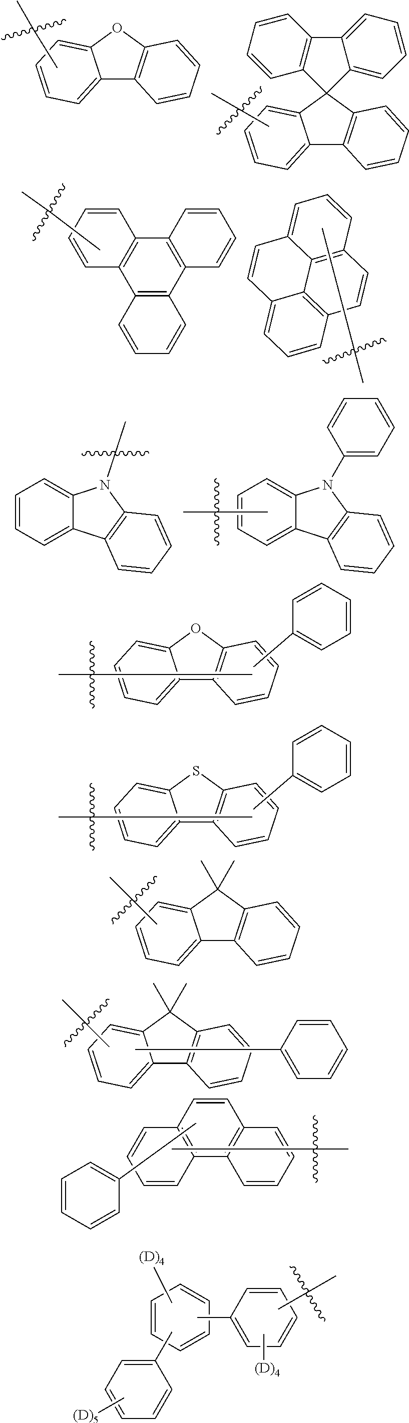

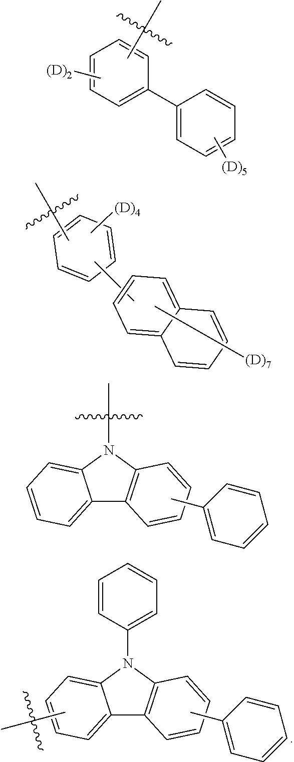

In some embodiments, Ar₁ and Ar₂ are respectively independently selected from the group consisting of the following groups:

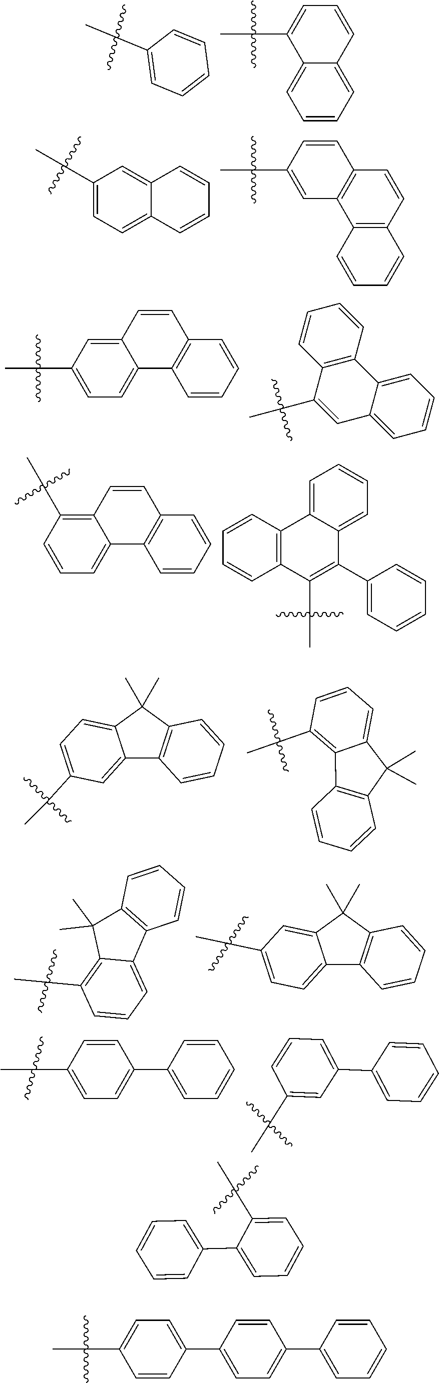

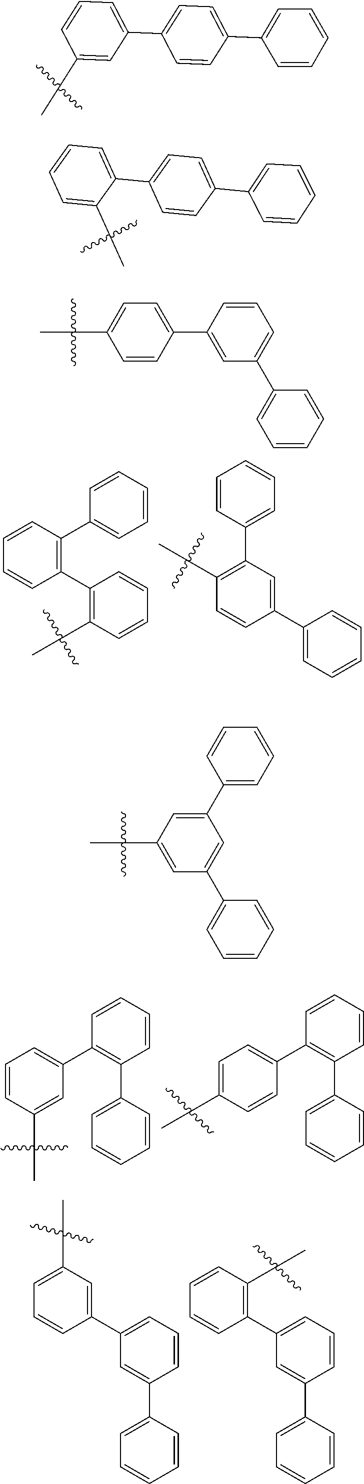

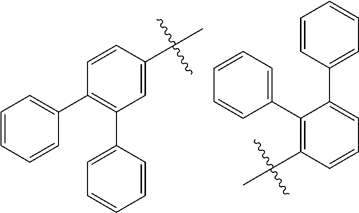

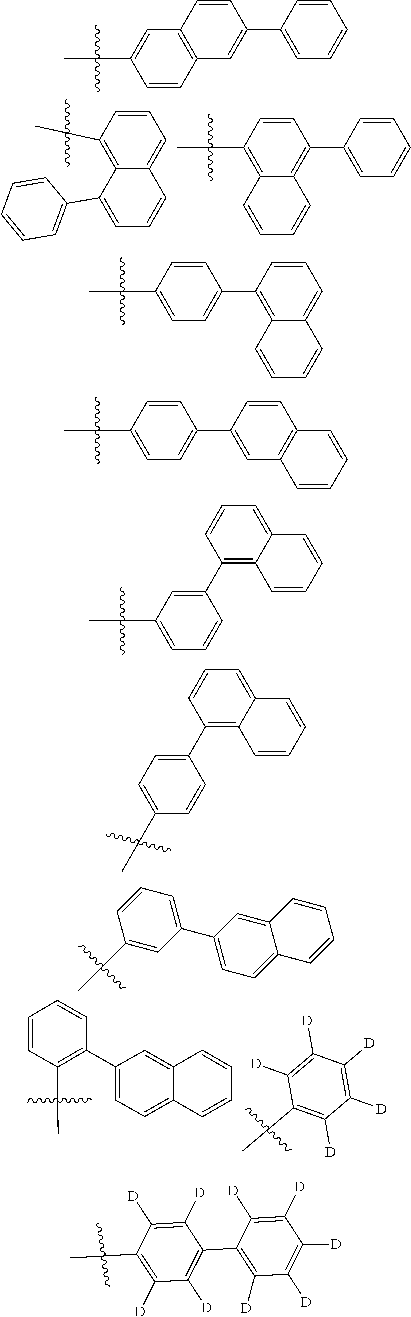

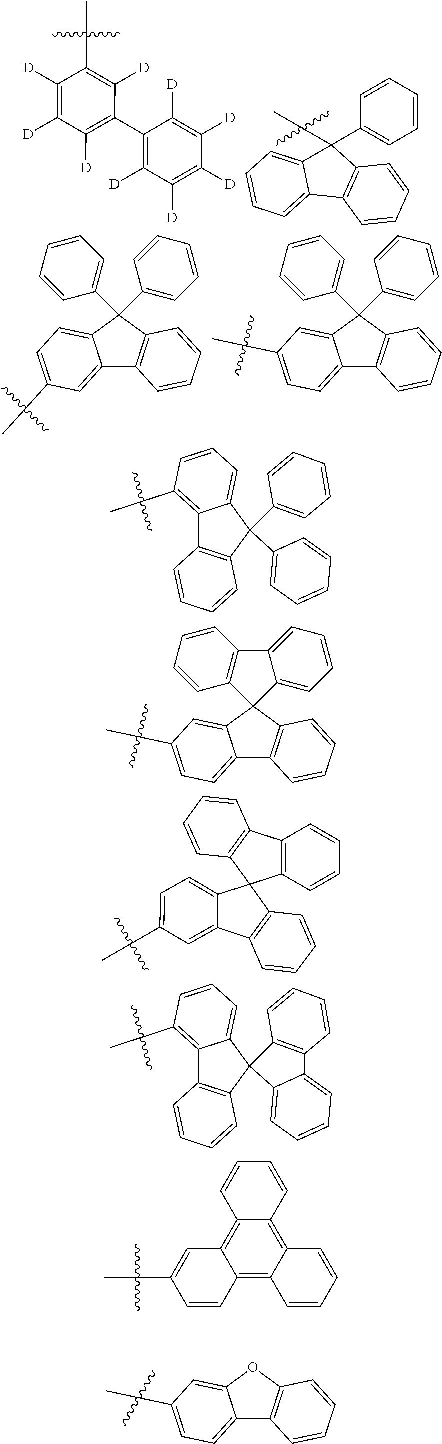

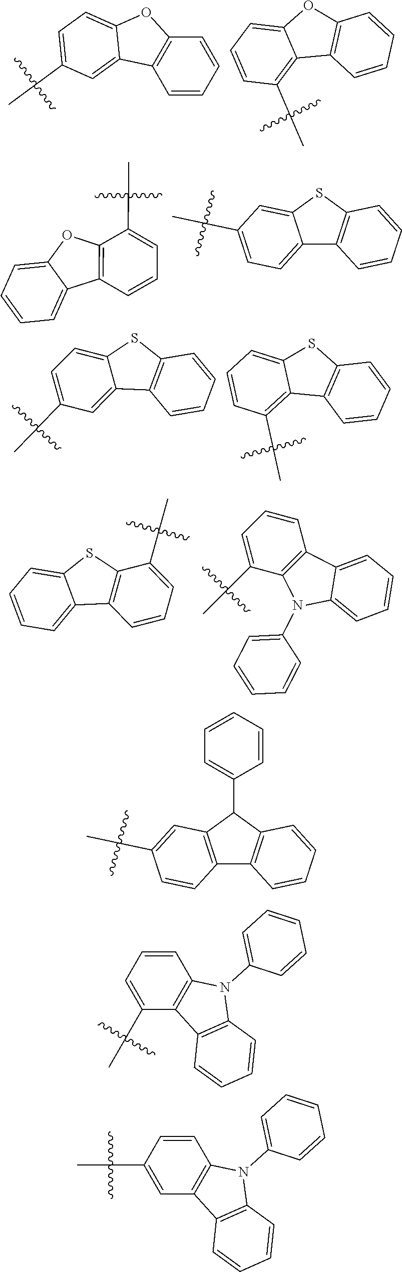

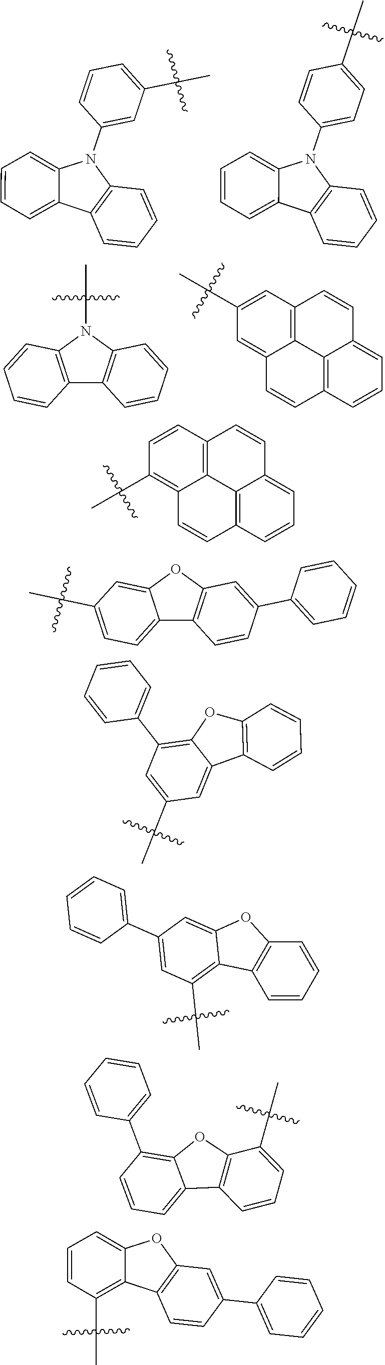

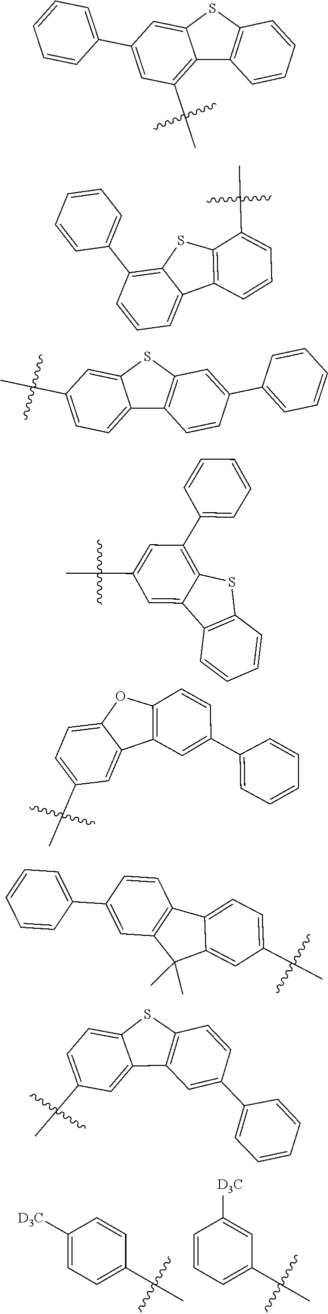

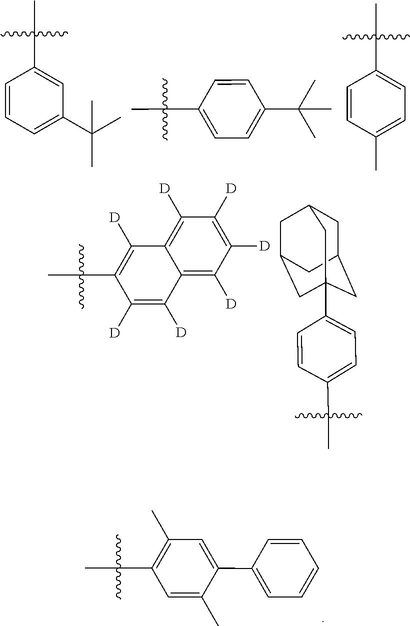

In some embodiments,

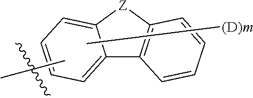

A in Formula 1 is selected from the following groups:

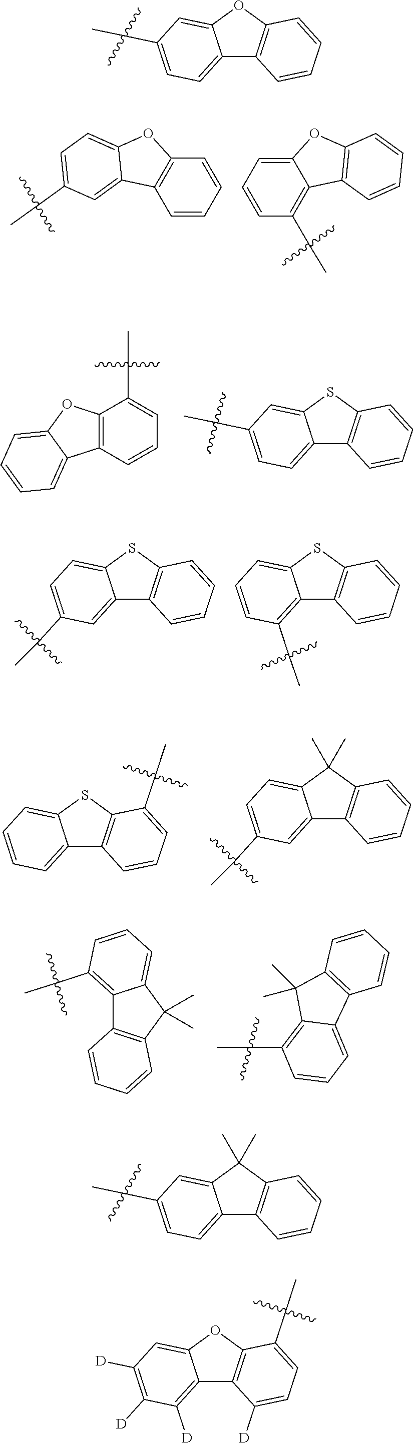

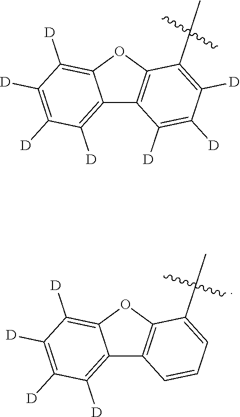 [SMILES: *c1c([2H])c([2H])c([2H])c2c1oc1c([2H])c([2H])c([2H])c([2H])c12 ; *c1cccc2c1oc1c([2H])c([2H])c([2H])c([2H])c12]

In some embodiments,

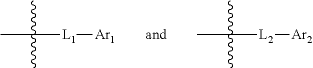

are each independently selected from the following groups:

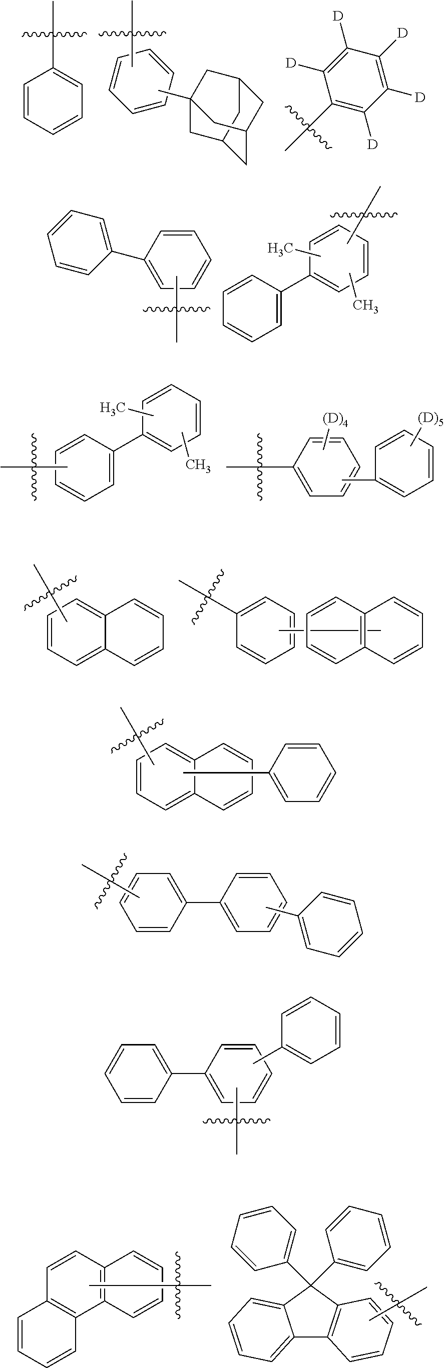

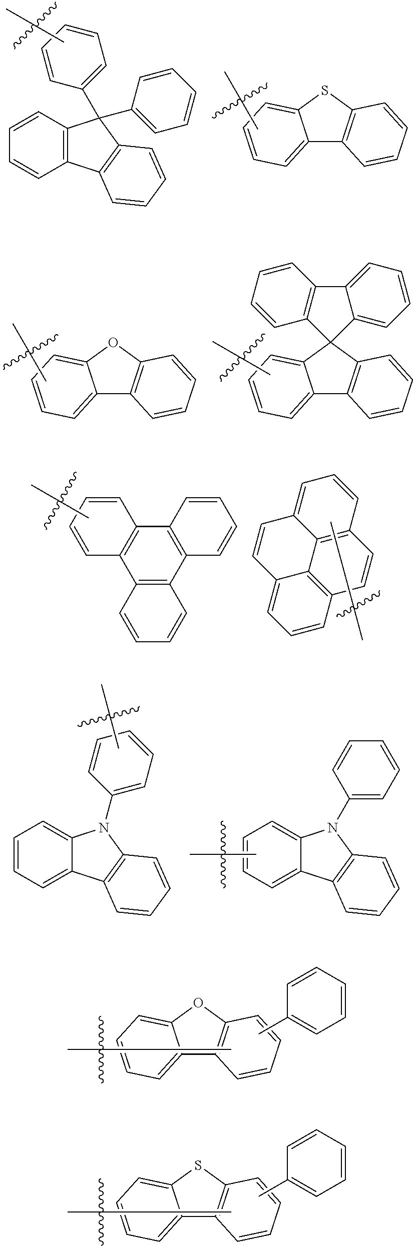

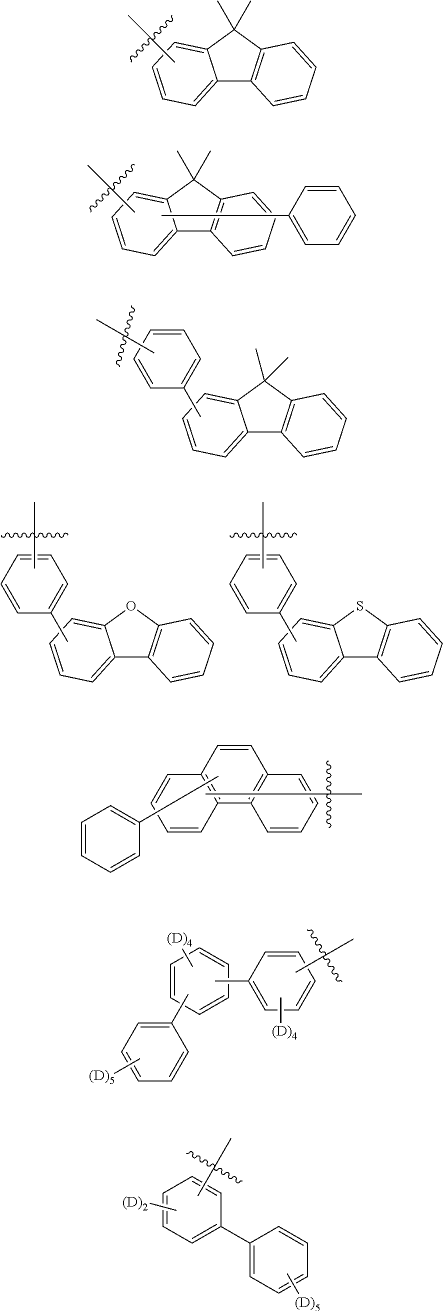

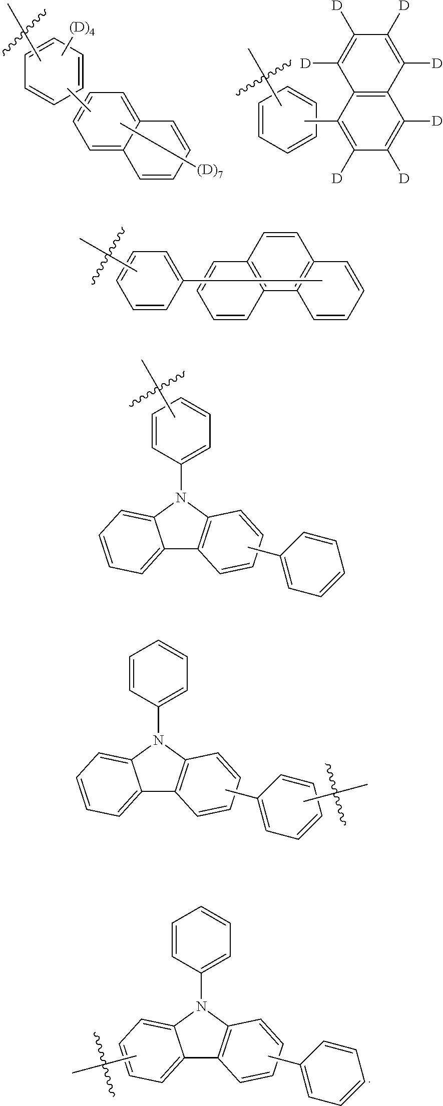

In some embodiments,

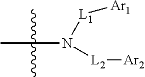

is selected from the following groups:

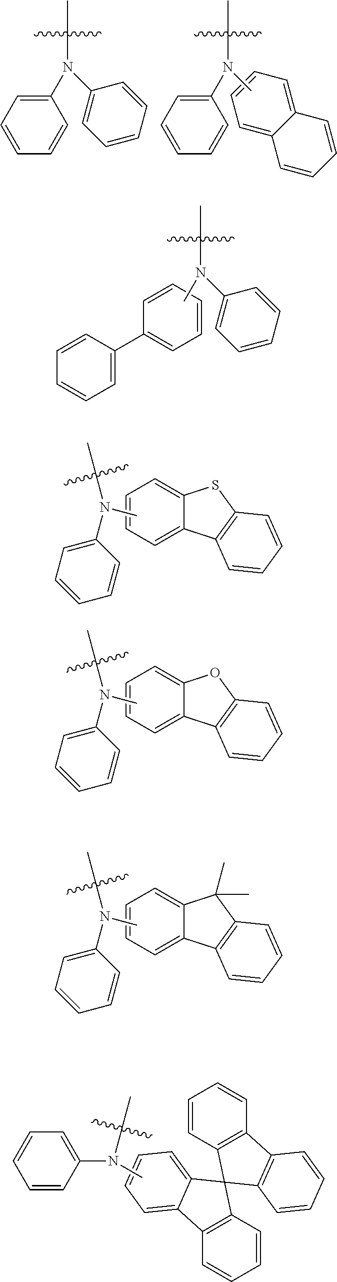

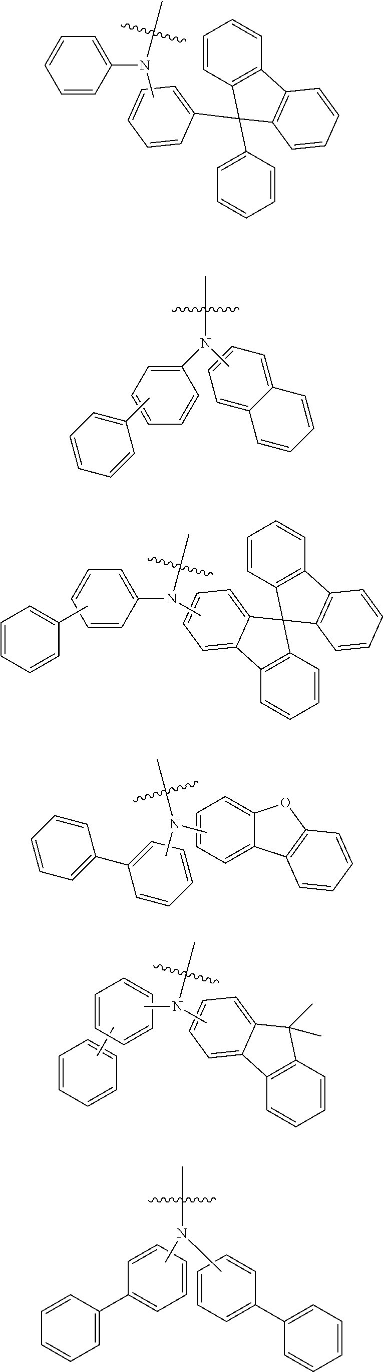

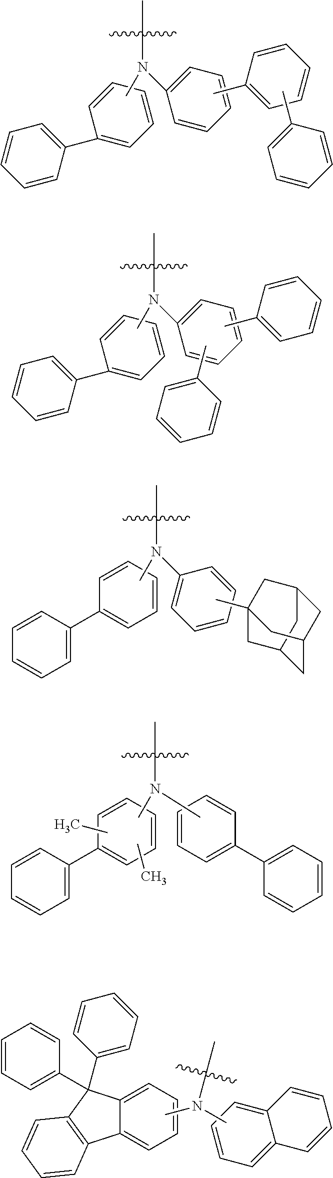

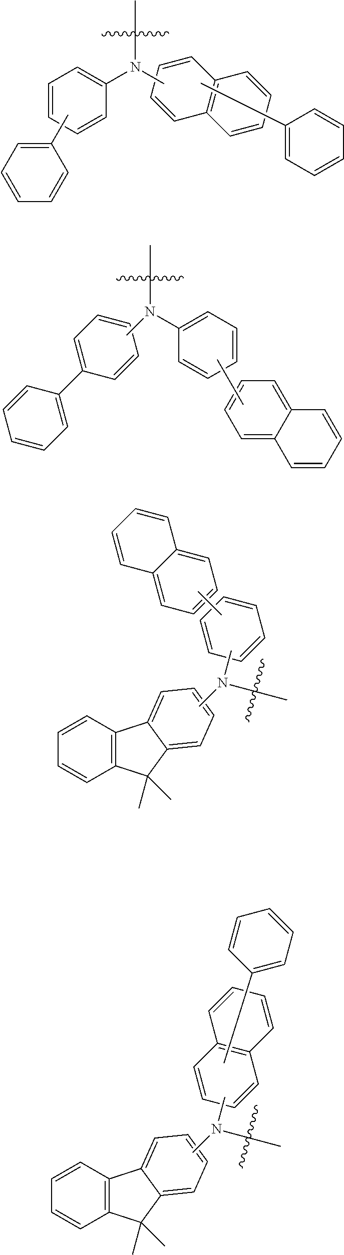

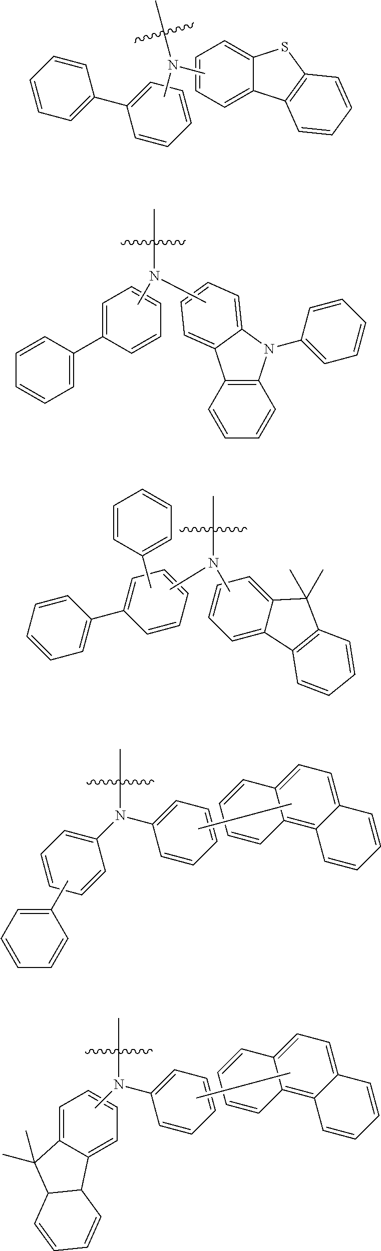

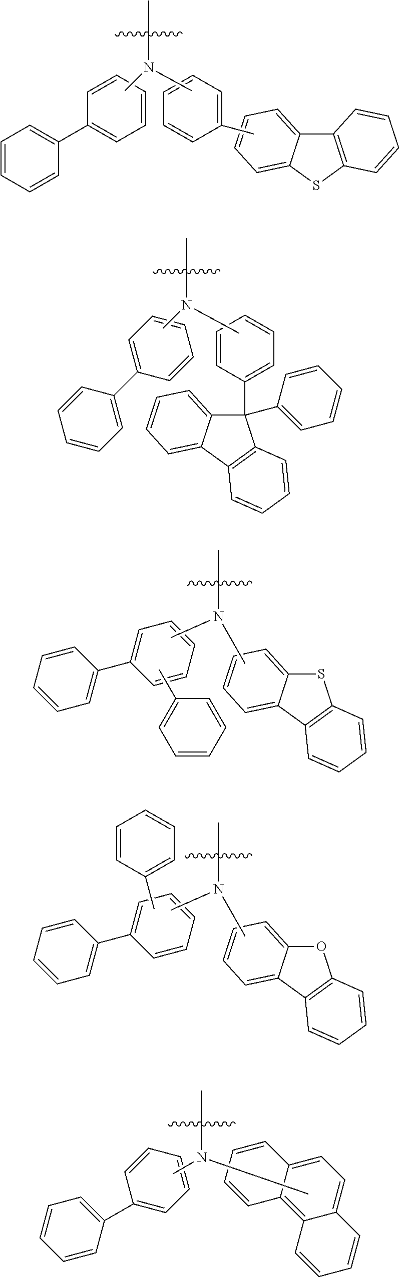

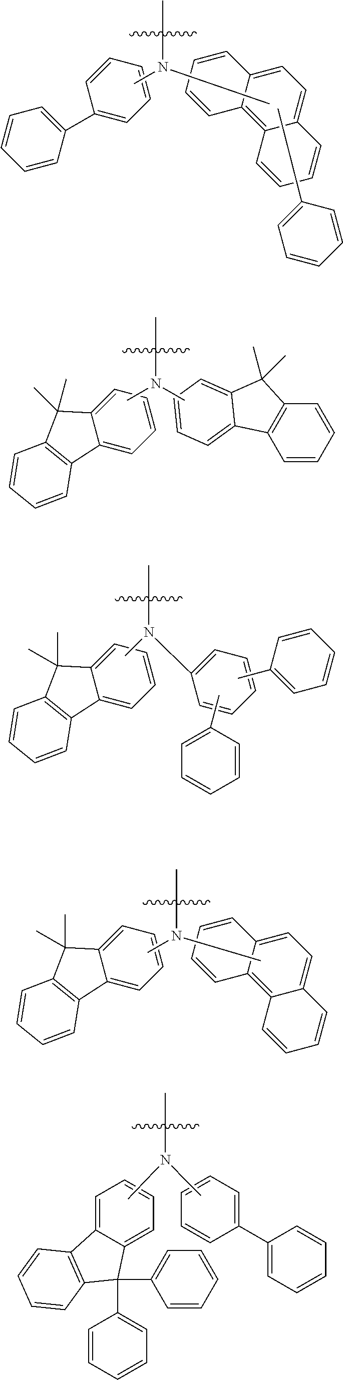

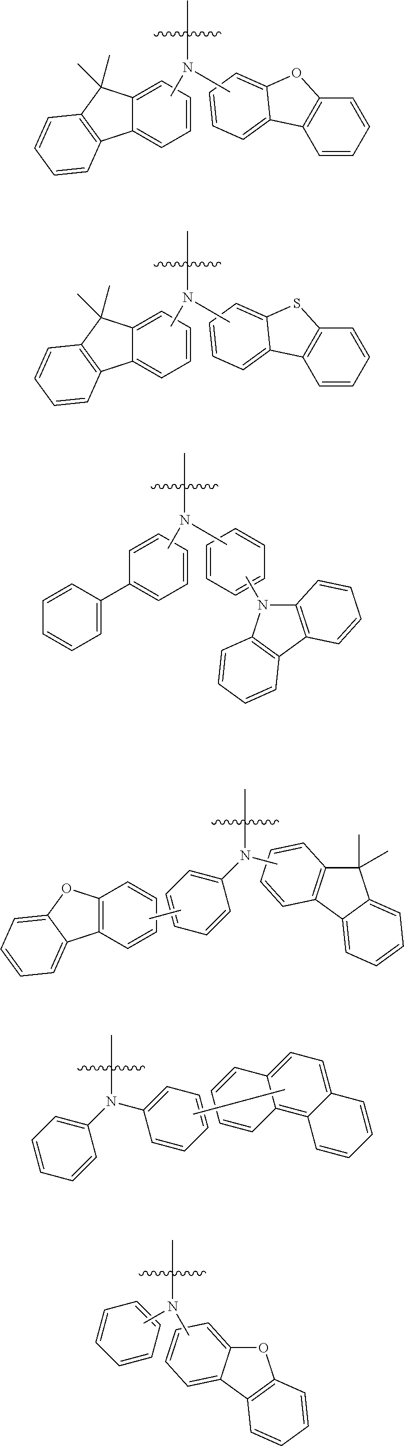

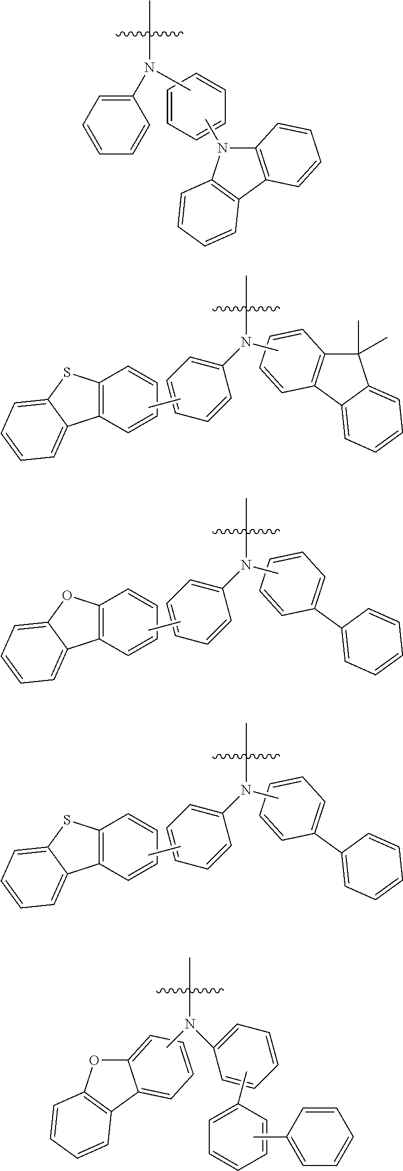

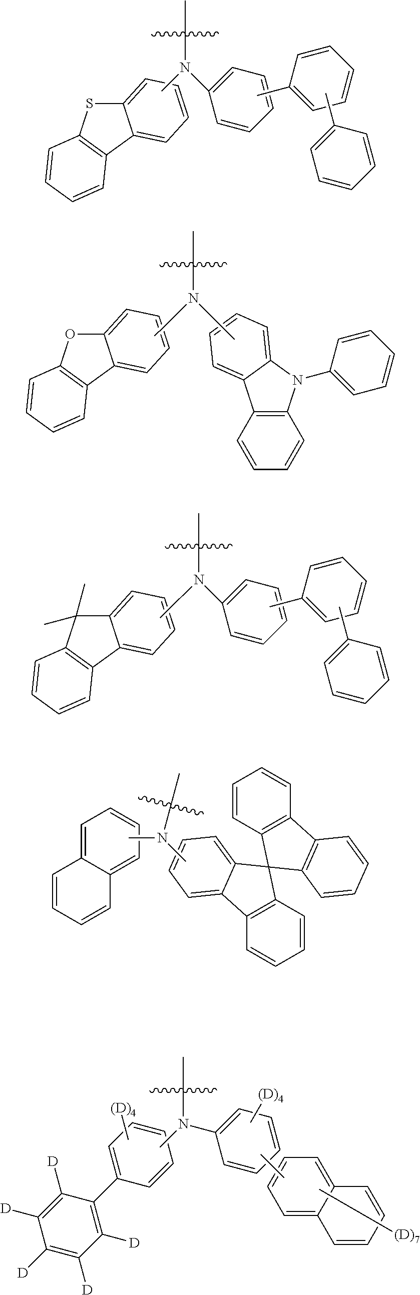

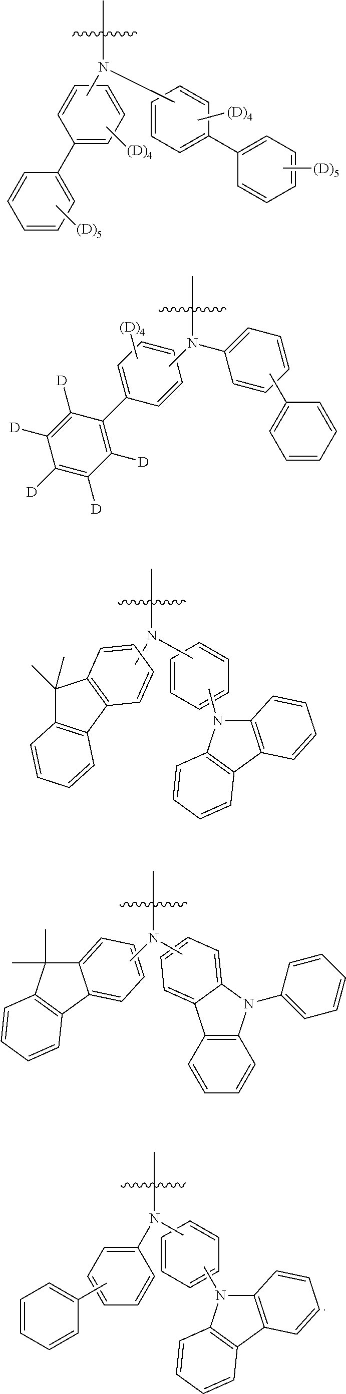

In some embodiments,

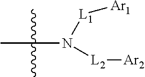

is selected from the following groups:

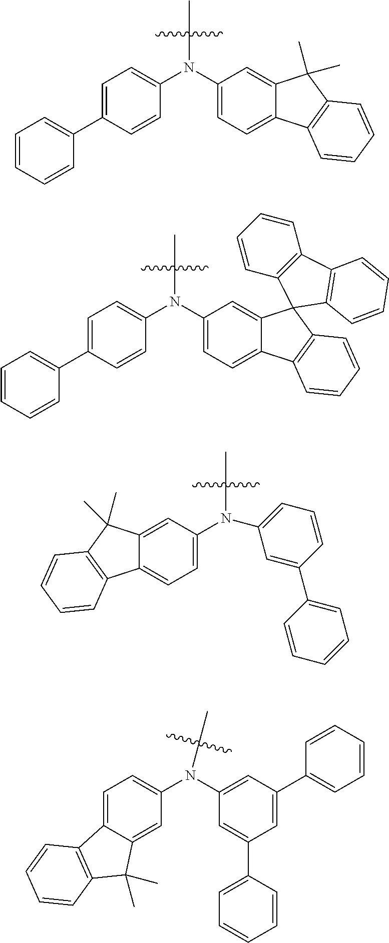 [SMILES: *N(c1ccc(-c2ccccc2)cc1)c1ccc2c(c1)C(C)(C)c1ccccc1-2 ; *N(c1ccc(-c2ccccc2)cc1)c1ccc2c(c1)C1(c3ccccc3-c3ccccc31)c1ccccc1-2 ; *N(c1cccc(-c2ccccc2)c1)c1ccc2c(c1)C(C)(C)c1ccccc1-2 ; *N(c1cc(-c2ccccc2)cc(-c2ccccc2)c1)c1ccc2c(c1)C(C)(C)c1ccccc1-2]

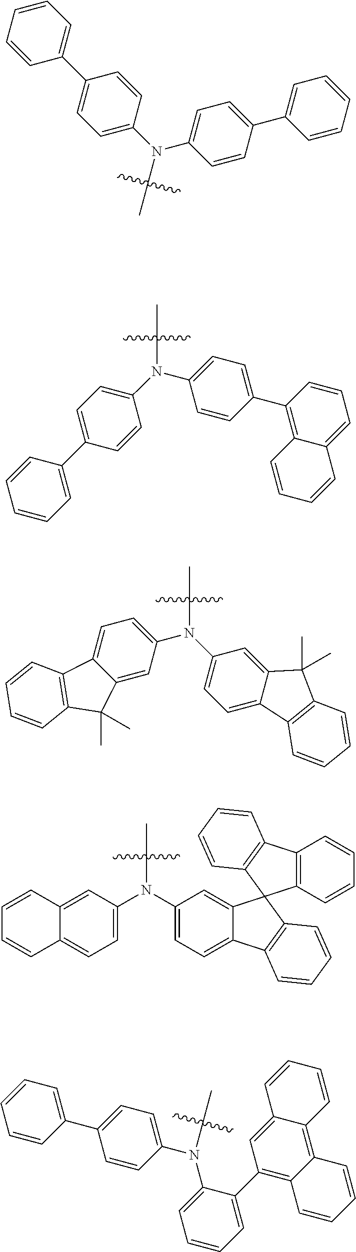 [SMILES: *N(c1ccc(-c2ccccc2)cc1)c1ccc(-c2ccccc2)cc1 ; *N(c1ccc(-c2ccccc2)cc1)c1ccc(-c2cccc3ccccc23)cc1 ; *N(c1ccc2c(c1)C(C)(C)c1ccccc1-2)c1ccc2c(c1)C(C)(C)c1ccccc1-2 ; *N(c1ccc2c(c1)C1(c3ccccc3-c3ccccc31)c1ccccc1-2)c1ccc2ccccc2c1 ; *N(c1ccc(-c2ccccc2)cc1)c1ccccc1-c1cc2ccccc2c2ccccc12]

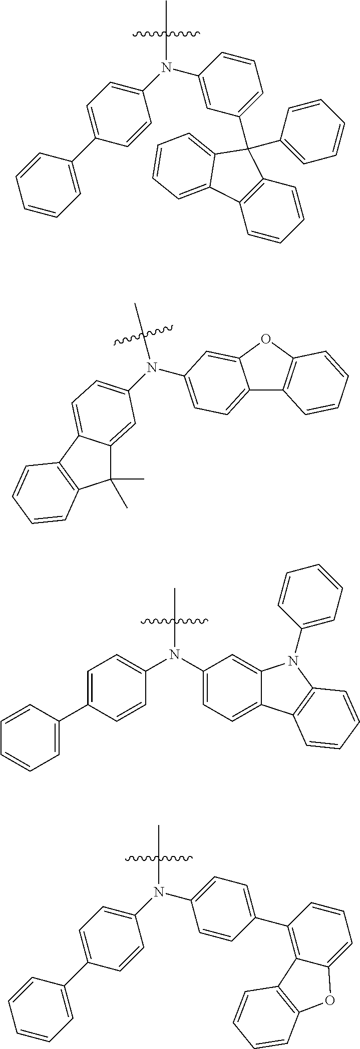 [SMILES: *N(c1ccc(-c2ccccc2)cc1)c1cccc(C2(c3ccccc3)c3ccccc3-c3ccccc32)c1 ; *N(c1ccc2c(c1)C(C)(C)c1ccccc1-2)c1ccc2c(c1)oc1ccccc12 ; *N(c1ccc(-c2ccccc2)cc1)c1ccc2c3ccccc3n(-c3ccccc3)c2c1 ; *N(c1ccc(-c2ccccc2)cc1)c1ccc(-c2cccc3oc4ccccc4c23)cc1]

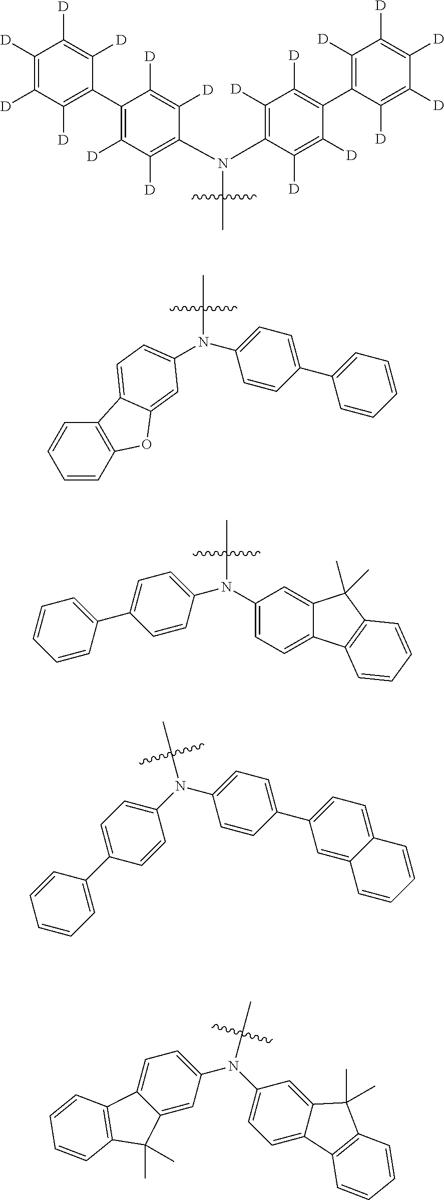 [SMILES: *N(c1c([2H])c([2H])c(-c2c([2H])c([2H])c([2H])c([2H])c2[2H])c([2H])c1[2H])c1c([2H])c([2H])c(-c2c([2H])c([2H])c([2H])c([2H])c2[2H])c([2H])c1[2H] ; *N(c1ccc(-c2ccccc2)cc1)c1ccc2c(c1)oc1ccccc12 ; *N(c1ccc(-c2ccccc2)cc1)c1ccc2c(c1)C(C)(C)c1ccccc1-2 ; *N(c1ccc(-c2ccccc2)cc1)c1ccc(-c2ccc3ccccc3c2)cc1 ; *N(c1ccc2c(c1)C(C)(C)c1ccccc1-2)c1ccc2c(c1)C(C)(C)c1ccccc1-2]

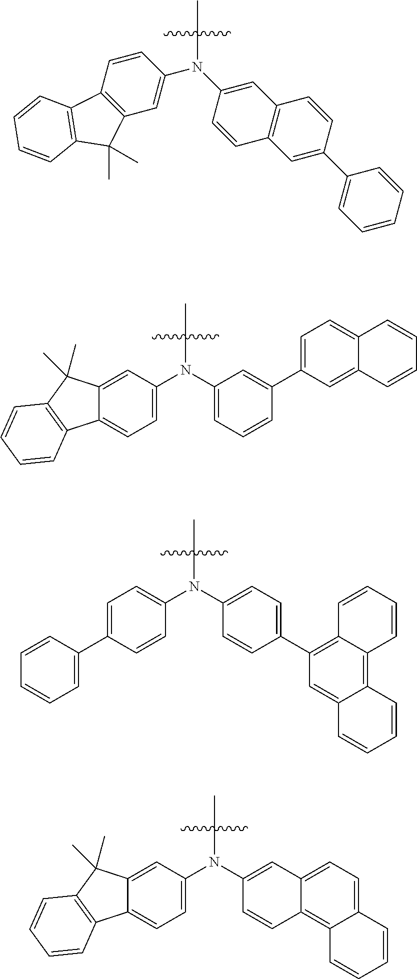 [SMILES: *N(c1ccc2c(c1)C(C)(C)c1ccccc1-2)c1ccc2cc(-c3ccccc3)ccc2c1 ; *N(c1cccc(-c2ccc3ccccc3c2)c1)c1ccc2c(c1)C(C)(C)c1ccccc1-2 ; *N(c1ccc(-c2ccccc2)cc1)c1ccc(-c2cc3ccccc3c3ccccc23)cc1 ; *N(c1ccc2c(c1)C(C)(C)c1ccccc1-2)c1ccc2c(ccc3ccccc32)c1]

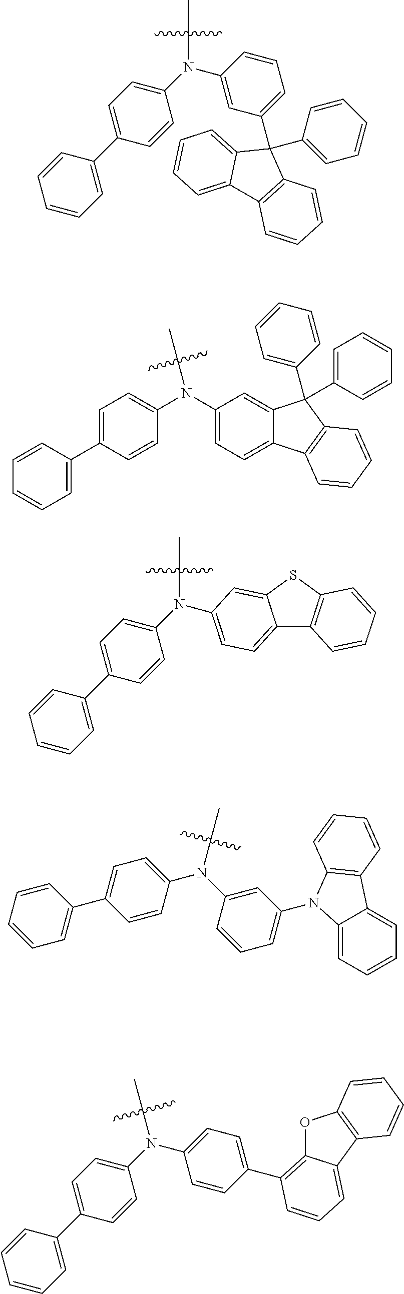 [SMILES: *N(c1ccc(-c2ccccc2)cc1)c1cccc(C2(c3ccccc3)c3ccccc3-c3ccccc32)c1 ; *N(c1ccc(-c2ccccc2)cc1)c1ccc2c(c1)C(c1ccccc1)(c1ccccc1)c1ccccc1-2 ; *N(c1ccc(-c2ccccc2)cc1)c1ccc2c(c1)sc1ccccc12 ; *N(c1ccc(-c2ccccc2)cc1)c1cccc(-n2c3ccccc3c3ccccc32)c1 ; *N(c1ccc(-c2ccccc2)cc1)c1ccc(-c2cccc3c2oc2ccccc23)cc1]

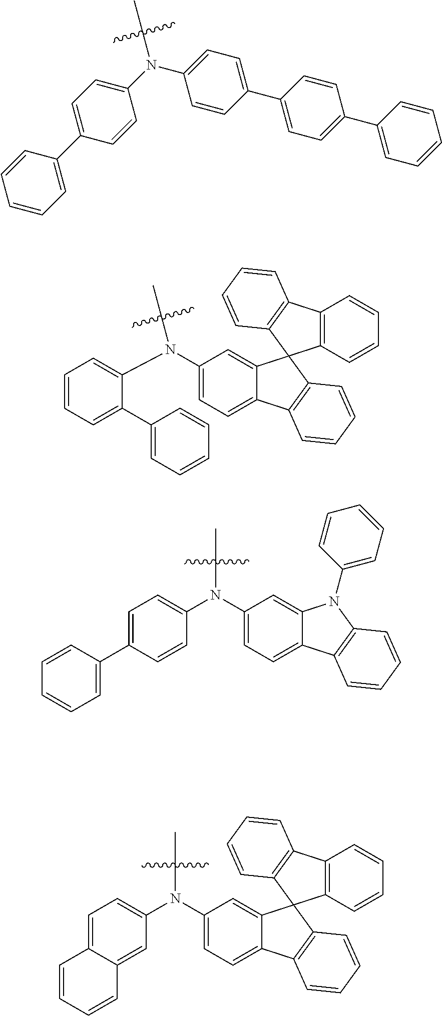 [SMILES: *N(c1ccc(-c2ccccc2)cc1)c1ccc(-c2ccc(-c3ccccc3)cc2)cc1 ; *N(c1ccc2c(c1)C1(c3ccccc3-c3ccccc31)c1ccccc1-2)c1ccccc1-c1ccccc1 ; *N(c1ccc(-c2ccccc2)cc1)c1ccc2c3ccccc3n(-c3ccccc3)c2c1 ; *N(c1ccc2c(c1)C1(c3ccccc3-c3ccccc31)c1ccccc1-2)c1ccc2ccccc2c1]

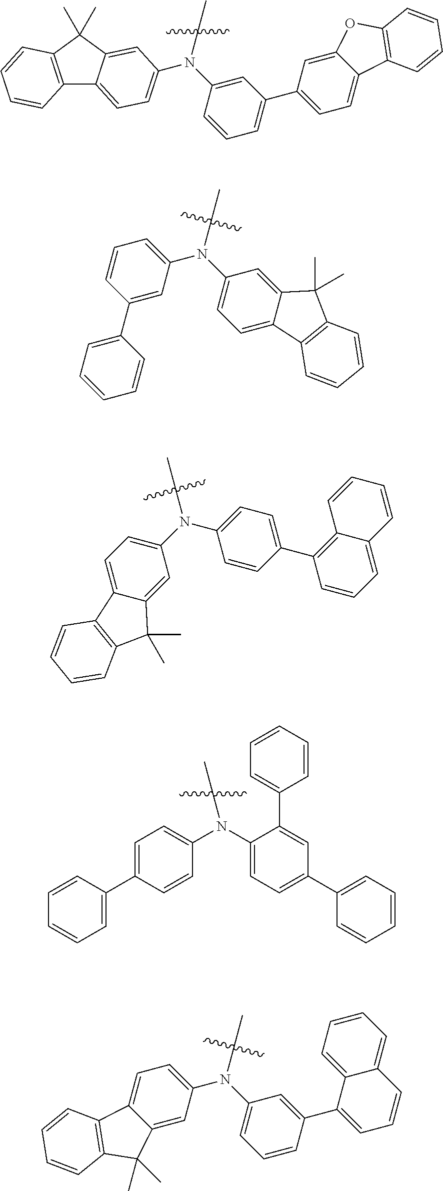 [SMILES: *N(c1cccc(-c2ccc3c(c2)oc2ccccc23)c1)c1ccc2c(c1)C(C)(C)c1ccccc1-2 ; *N(c1cccc(-c2ccccc2)c1)c1ccc2c(c1)C(C)(C)c1ccccc1-2 ; *N(c1ccc(-c2cccc3ccccc23)cc1)c1ccc2c(c1)C(C)(C)c1ccccc1-2 ; *N(c1ccc(-c2ccccc2)cc1)c1ccc(-c2ccccc2)cc1-c1ccccc1 ; *N(c1cccc(-c2cccc3ccccc23)c1)c1ccc2c(c1)C(C)(C)c1ccccc1-2]

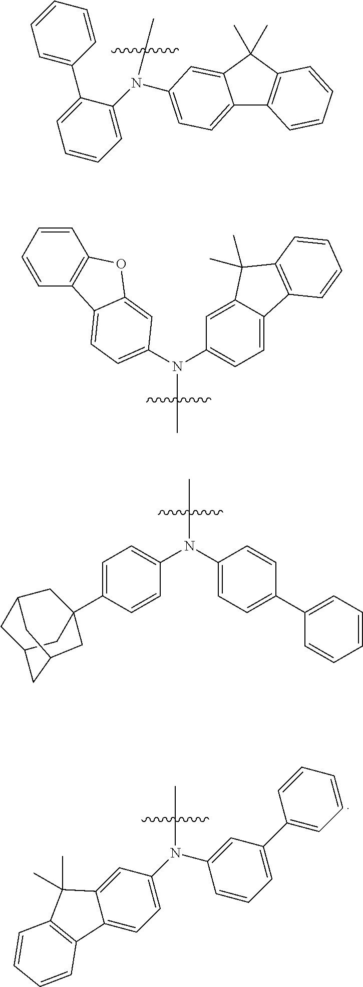 [SMILES: *N(c1ccc2c(c1)C(C)(C)c1ccccc1-2)c1ccccc1-c1ccccc1 ; *N(c1ccc2c(c1)C(C)(C)c1ccccc1-2)c1ccc2c(c1)oc1ccccc12 ; *N(c1ccc(-c2ccccc2)cc1)c1ccc(C23CC4CC(CC(C4)C2)C3)cc1 ; *N(c1cccc(-c2ccccc2)c1)c1ccc2c(c1)C(C)(C)c1ccccc1-2]

In some embodiments, the arylamine compound is selected from the following compounds as follows:

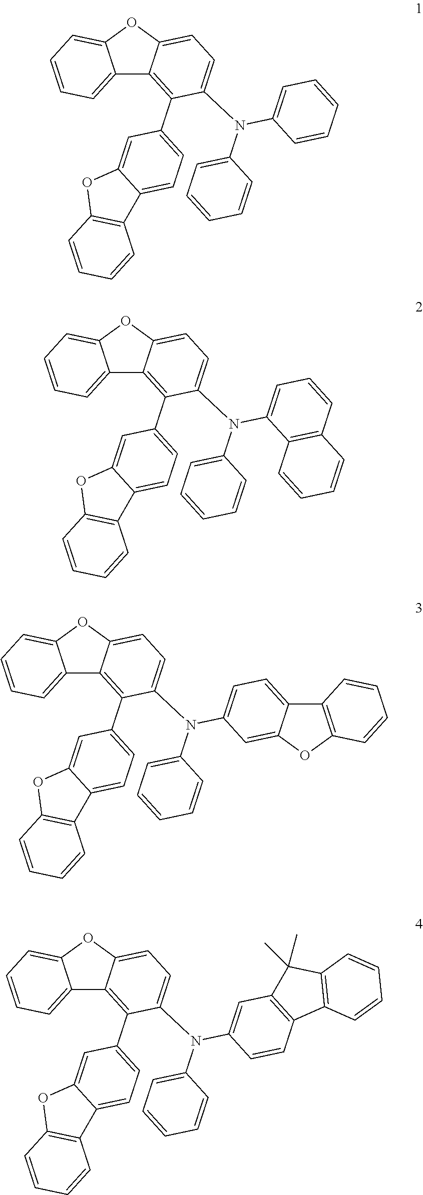

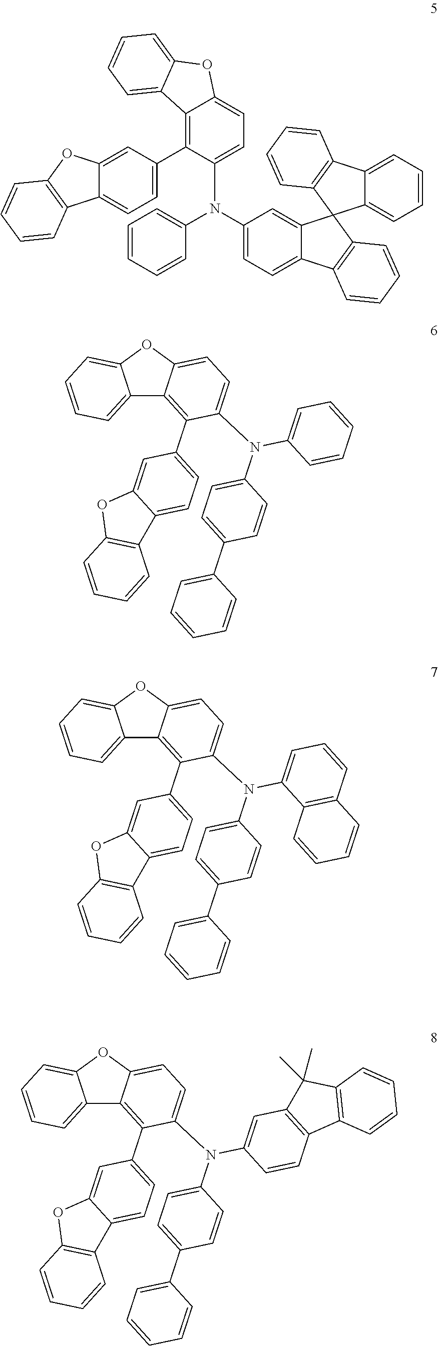 [SMILES: c1ccc(N(c2ccc3c(c2)C2(c4ccccc4-c4ccccc42)c2ccccc2-3)c2ccc3oc4ccccc4c3c2-c2ccc3c(c2)oc2ccccc23)cc1 ; c1ccc(-c2ccc(N(c3ccccc3)c3ccc4oc5ccccc5c4c3-c3ccc4c(c3)oc3ccccc34)cc2)cc1 ; c1ccc(-c2ccc(N(c3ccc4oc5ccccc5c4c3-c3ccc4c(c3)oc3ccccc34)c3cccc4ccccc34)cc2)cc1 ; CC1(C)c2ccccc2-c2ccc(N(c3ccc(-c4ccccc4)cc3)c3ccc4oc5ccccc5c4c3-c3ccc4c(c3)oc3ccccc34)cc21]

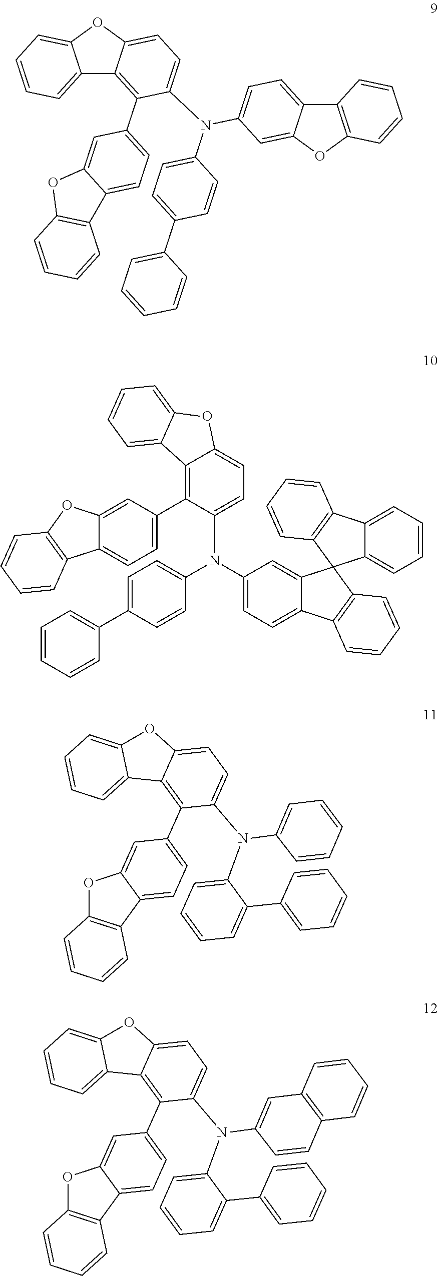 [SMILES: c1ccc(-c2ccc(N(c3ccc4c(c3)oc3ccccc34)c3ccc4oc5ccccc5c4c3-c3ccc4c(c3)oc3ccccc34)cc2)cc1 ; c1ccc(-c2ccc(N(c3ccc4c(c3)C3(c5ccccc5-c5ccccc53)c3ccccc3-4)c3ccc4oc5ccccc5c4c3-c3ccc4c(c3)oc3ccccc34)cc2)cc1 ; c1ccc(-c2ccccc2N(c2ccccc2)c2ccc3oc4ccccc4c3c2-c2ccc3c(c2)oc2ccccc23)cc1 ; c1ccc(-c2ccccc2N(c2ccc3ccccc3c2)c2ccc3oc4ccccc4c3c2-c2ccc3c(c2)oc2ccccc23)cc1]

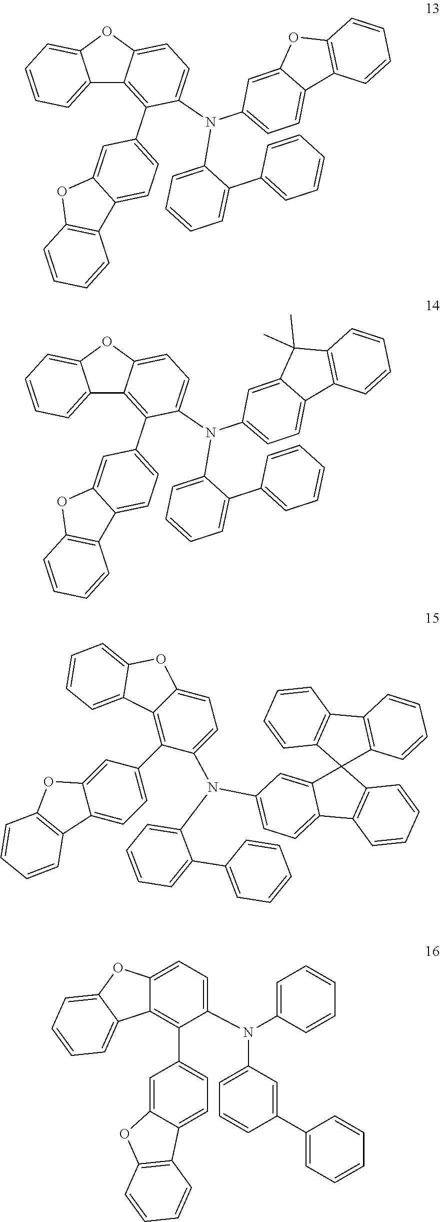 [SMILES: c1ccc(-c2ccccc2N(c2ccc3c(c2)oc2ccccc23)c2ccc3oc4ccccc4c3c2-c2ccc3c(c2)oc2ccccc23)cc1 ; CC1(C)c2ccccc2-c2ccc(N(c3ccccc3-c3ccccc3)c3ccc4oc5ccccc5c4c3-c3ccc4c(c3)oc3ccccc34)cc21 ; c1ccc(-c2ccccc2N(c2ccc3c(c2)C2(c4ccccc4-c4ccccc42)c2ccccc2-3)c2ccc3oc4ccccc4c3c2-c2ccc3c(c2)oc2ccccc23)cc1 ; c1ccc(-c2cccc(N(c3ccccc3)c3ccc4oc5ccccc5c4c3-c3ccc4c(c3)oc3ccccc34)c2)cc1]

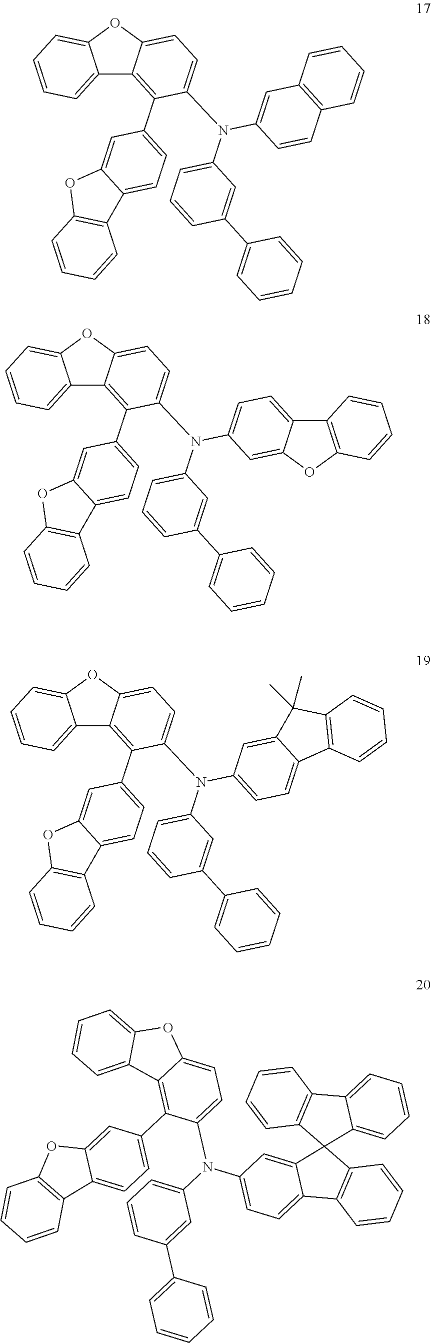 [SMILES: c1ccc(-c2cccc(N(c3ccc4ccccc4c3)c3ccc4oc5ccccc5c4c3-c3ccc4c(c3)oc3ccccc34)c2)cc1 ; c1ccc(-c2cccc(N(c3ccc4c(c3)oc3ccccc34)c3ccc4oc5ccccc5c4c3-c3ccc4c(c3)oc3ccccc34)c2)cc1 ; CC1(C)c2ccccc2-c2ccc(N(c3cccc(-c4ccccc4)c3)c3ccc4oc5ccccc5c4c3-c3ccc4c(c3)oc3ccccc34)cc21 ; c1ccc(-c2cccc(N(c3ccc4c(c3)C3(c5ccccc5-c5ccccc53)c3ccccc3-4)c3ccc4oc5ccccc5c4c3-c3ccc4c(c3)oc3ccccc34)c2)cc1]

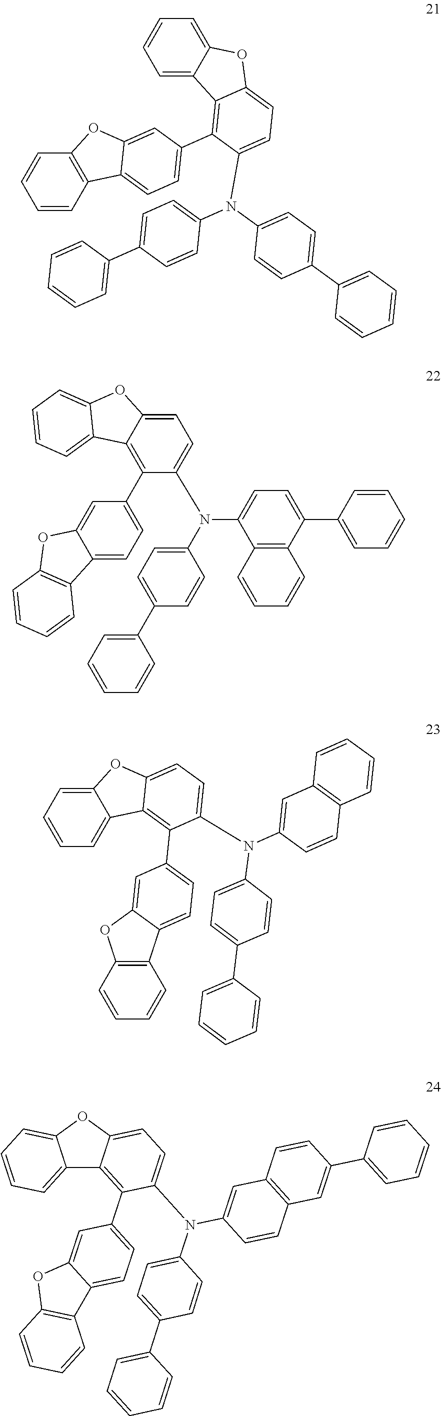 [SMILES: c1ccc(-c2ccc(N(c3ccc(-c4ccccc4)cc3)c3ccc4oc5ccccc5c4c3-c3ccc4c(c3)oc3ccccc34)cc2)cc1 ; c1ccc(-c2ccc(N(c3ccc4oc5ccccc5c4c3-c3ccc4c(c3)oc3ccccc34)c3ccc(-c4ccccc4)c4ccccc34)cc2)cc1 ; c1ccc(-c2ccc(N(c3ccc4ccccc4c3)c3ccc4oc5ccccc5c4c3-c3ccc4c(c3)oc3ccccc34)cc2)cc1 ; c1ccc(-c2ccc(N(c3ccc4cc(-c5ccccc5)ccc4c3)c3ccc4oc5ccccc5c4c3-c3ccc4c(c3)oc3ccccc34)cc2)cc1]

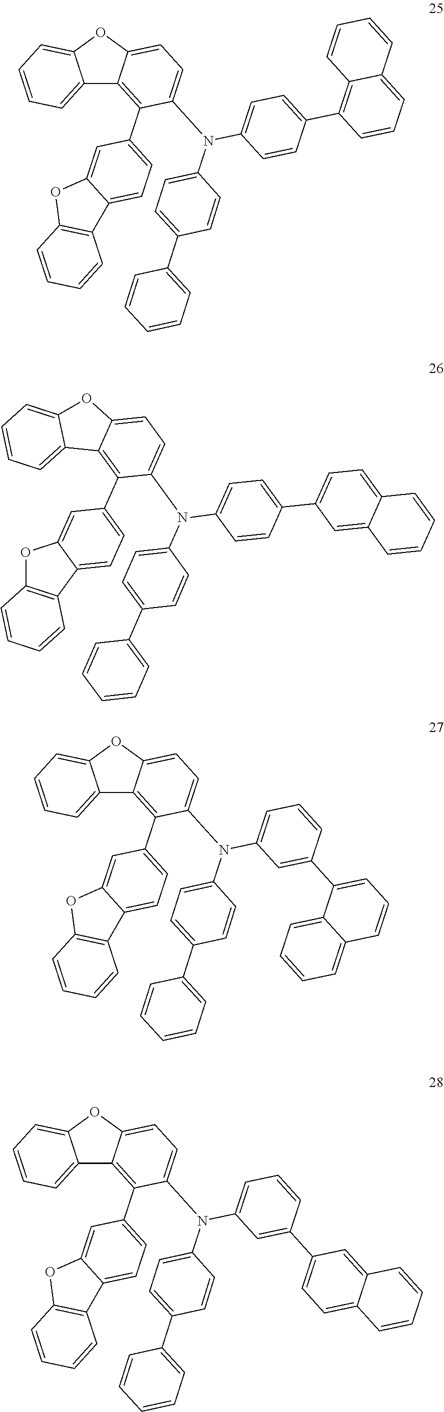

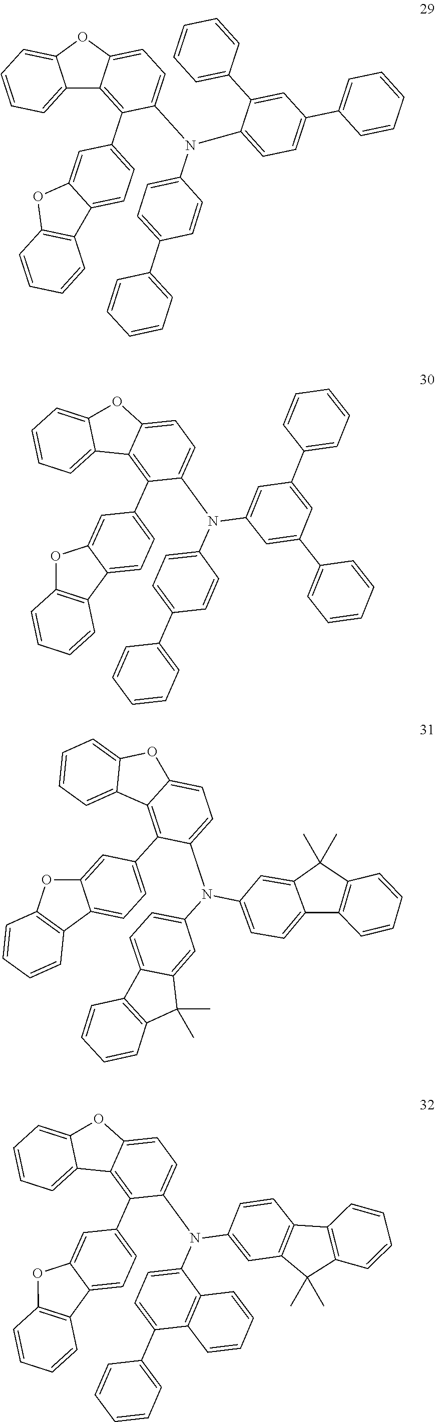 [SMILES: c1ccc(-c2ccc(N(c3ccc(-c4ccccc4)cc3-c3ccccc3)c3ccc4oc5ccccc5c4c3-c3ccc4c(c3)oc3ccccc34)cc2)cc1 ; c1ccc(-c2ccc(N(c3cc(-c4ccccc4)cc(-c4ccccc4)c3)c3ccc4oc5ccccc5c4c3-c3ccc4c(c3)oc3ccccc34)cc2)cc1 ; CC1(C)c2ccccc2-c2ccc(N(c3ccc4c(c3)C(C)(C)c3ccccc3-4)c3ccc4oc5ccccc5c4c3-c3ccc4c(c3)oc3ccccc34)cc21 ; CC1(C)c2ccccc2-c2ccc(N(c3ccc4oc5ccccc5c4c3-c3ccc4c(c3)oc3ccccc34)c3ccc(-c4ccccc4)c4ccccc34)cc21]

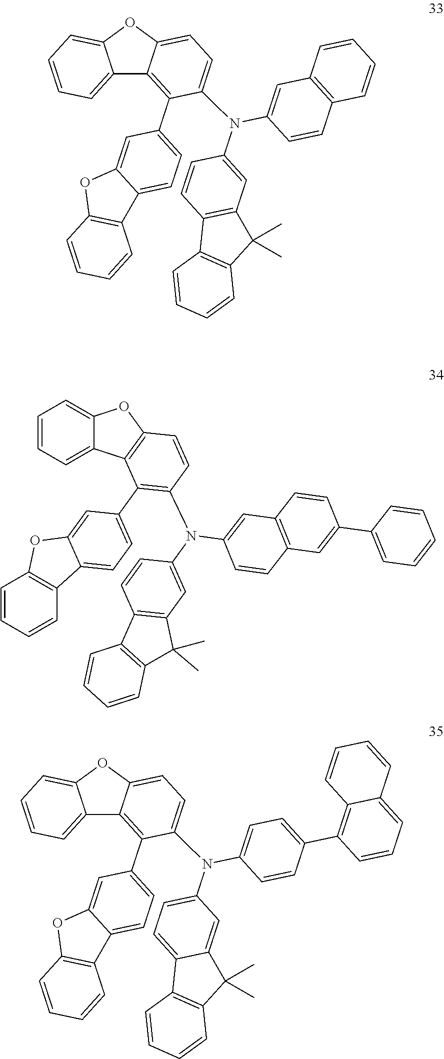 [SMILES: CC1(C)c2ccccc2-c2ccc(N(c3ccc4ccccc4c3)c3ccc4oc5ccccc5c4c3-c3ccc4c(c3)oc3ccccc34)cc21 ; CC1(C)c2ccccc2-c2ccc(N(c3ccc4cc(-c5ccccc5)ccc4c3)c3ccc4oc5ccccc5c4c3-c3ccc4c(c3)oc3ccccc34)cc21 ; CC1(C)c2ccccc2-c2ccc(N(c3ccc(-c4cccc5ccccc45)cc3)c3ccc4oc5ccccc5c4c3-c3ccc4c(c3)oc3ccccc34)cc21]

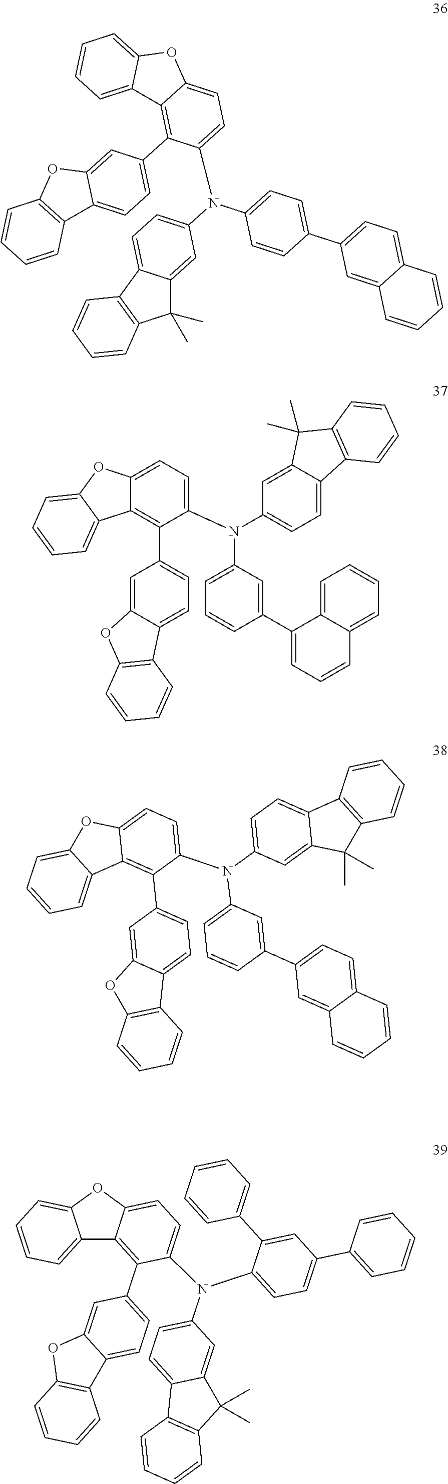 [SMILES: CC1(C)c2ccccc2-c2ccc(N(c3ccc(-c4ccc5ccccc5c4)cc3)c3ccc4oc5ccccc5c4c3-c3ccc4c(c3)oc3ccccc34)cc21 ; CC1(C)c2ccccc2-c2ccc(N(c3cccc(-c4cccc5ccccc45)c3)c3ccc4oc5ccccc5c4c3-c3ccc4c(c3)oc3ccccc34)cc21 ; CC1(C)c2ccccc2-c2ccc(N(c3cccc(-c4ccc5ccccc5c4)c3)c3ccc4oc5ccccc5c4c3-c3ccc4c(c3)oc3ccccc34)cc21 ; CC1(C)c2ccccc2-c2ccc(N(c3ccc(-c4ccccc4)cc3-c3ccccc3)c3ccc4oc5ccccc5c4c3-c3ccc4c(c3)oc3ccccc34)cc21]

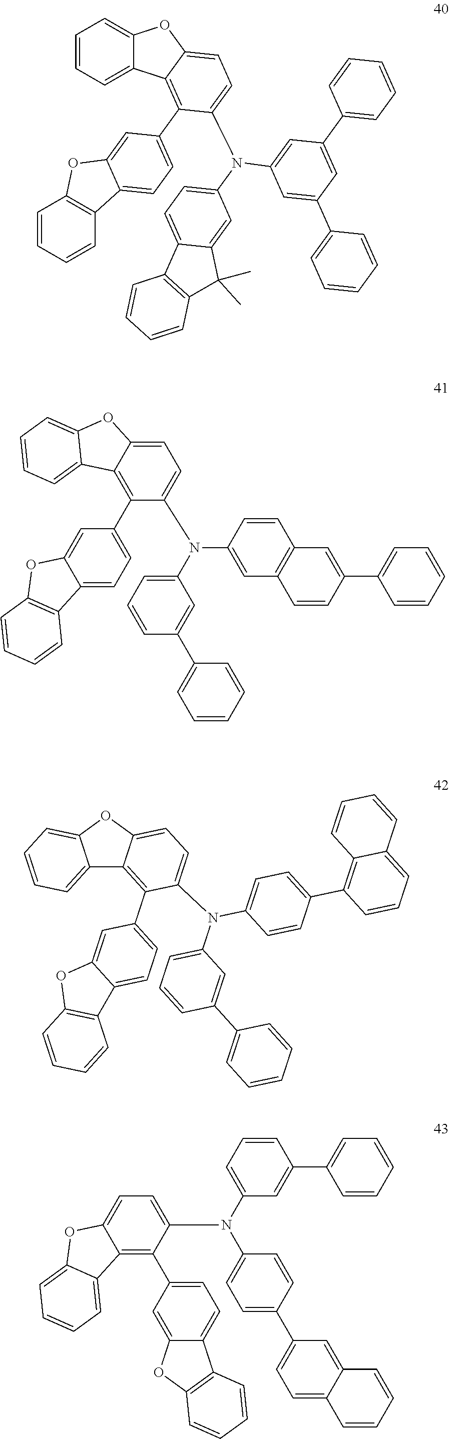 [SMILES: CC1(C)c2ccccc2-c2ccc(N(c3cc(-c4ccccc4)cc(-c4ccccc4)c3)c3ccc4oc5ccccc5c4c3-c3ccc4c(c3)oc3ccccc34)cc21 ; c1ccc(-c2cccc(N(c3ccc4cc(-c5ccccc5)ccc4c3)c3ccc4oc5ccccc5c4c3-c3ccc4c(c3)oc3ccccc34)c2)cc1 ; c1ccc(-c2cccc(N(c3ccc(-c4cccc5ccccc45)cc3)c3ccc4oc5ccccc5c4c3-c3ccc4c(c3)oc3ccccc34)c2)cc1 ; c1ccc(-c2cccc(N(c3ccc(-c4ccc5ccccc5c4)cc3)c3ccc4oc5ccccc5c4c3-c3ccc4c(c3)oc3ccccc34)c2)cc1]

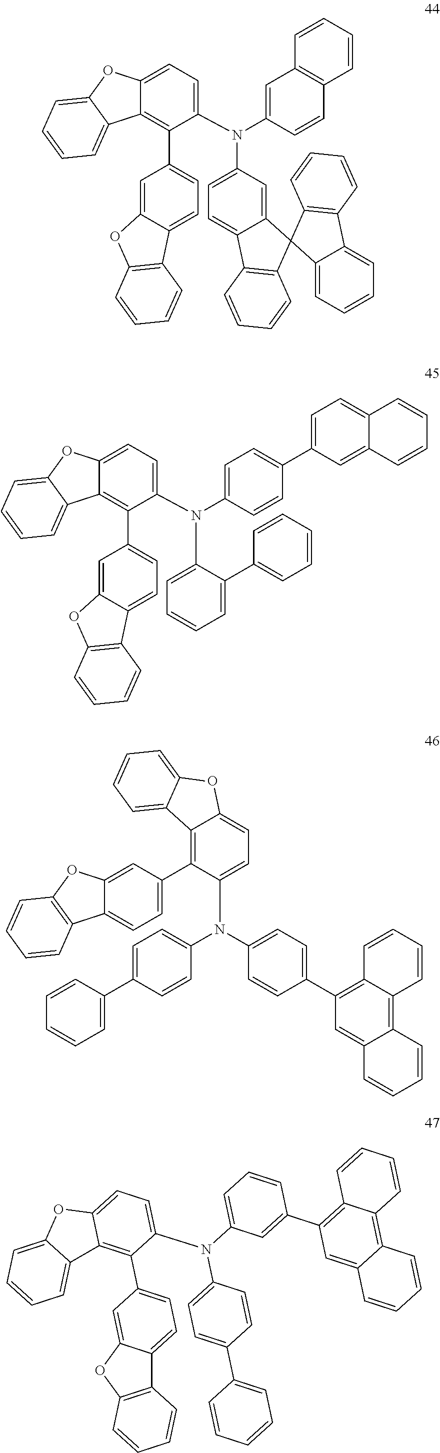 [SMILES: c1ccc2c(c1)-c1ccccc1C21c2ccccc2-c2ccc(N(c3ccc4ccccc4c3)c3ccc4oc5ccccc5c4c3-c3ccc4c(c3)oc3ccccc34)cc21 ; c1ccc(-c2ccccc2N(c2ccc(-c3ccc4ccccc4c3)cc2)c2ccc3oc4ccccc4c3c2-c2ccc3c(c2)oc2ccccc23)cc1 ; c1ccc(-c2ccc(N(c3ccc(-c4cc5ccccc5c5ccccc45)cc3)c3ccc4oc5ccccc5c4c3-c3ccc4c(c3)oc3ccccc34)cc2)cc1 ; c1ccc(-c2ccc(N(c3cccc(-c4cc5ccccc5c5ccccc45)c3)c3ccc4oc5ccccc5c4c3-c3ccc4c(c3)oc3ccccc34)cc2)cc1]

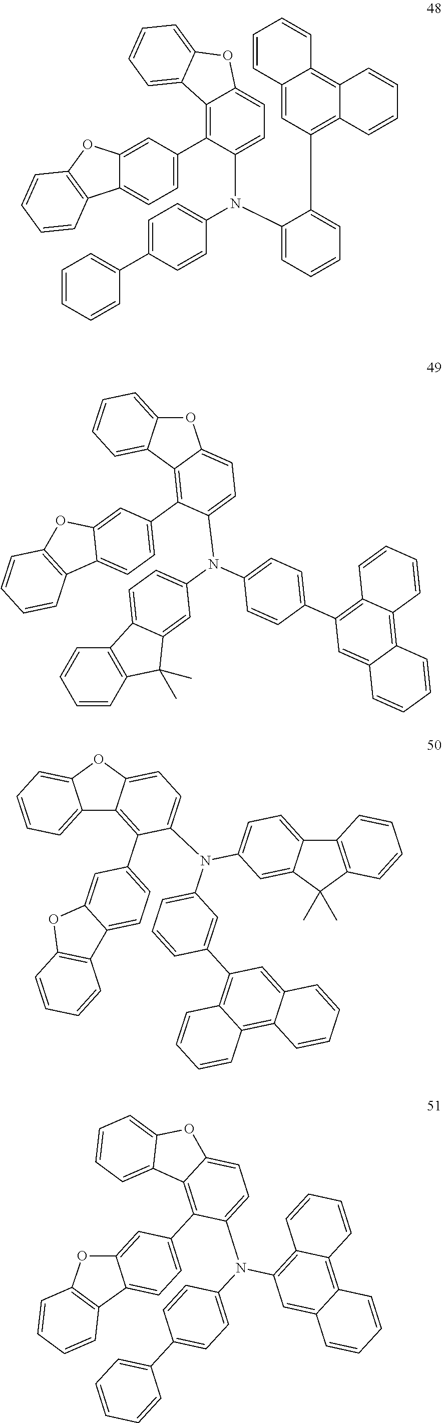 [SMILES: c1ccc(-c2ccc(N(c3ccccc3-c3cc4ccccc4c4ccccc34)c3ccc4oc5ccccc5c4c3-c3ccc4c(c3)oc3ccccc34)cc2)cc1 ; CC1(C)c2ccccc2-c2ccc(N(c3ccc(-c4cc5ccccc5c5ccccc45)cc3)c3ccc4oc5ccccc5c4c3-c3ccc4c(c3)oc3ccccc34)cc21 ; CC1(C)c2ccccc2-c2ccc(N(c3cccc(-c4cc5ccccc5c5ccccc45)c3)c3ccc4oc5ccccc5c4c3-c3ccc4c(c3)oc3ccccc34)cc21 ; c1ccc(-c2ccc(N(c3ccc4oc5ccccc5c4c3-c3ccc4c(c3)oc3ccccc34)c3cc4ccccc4c4ccccc34)cc2)cc1]

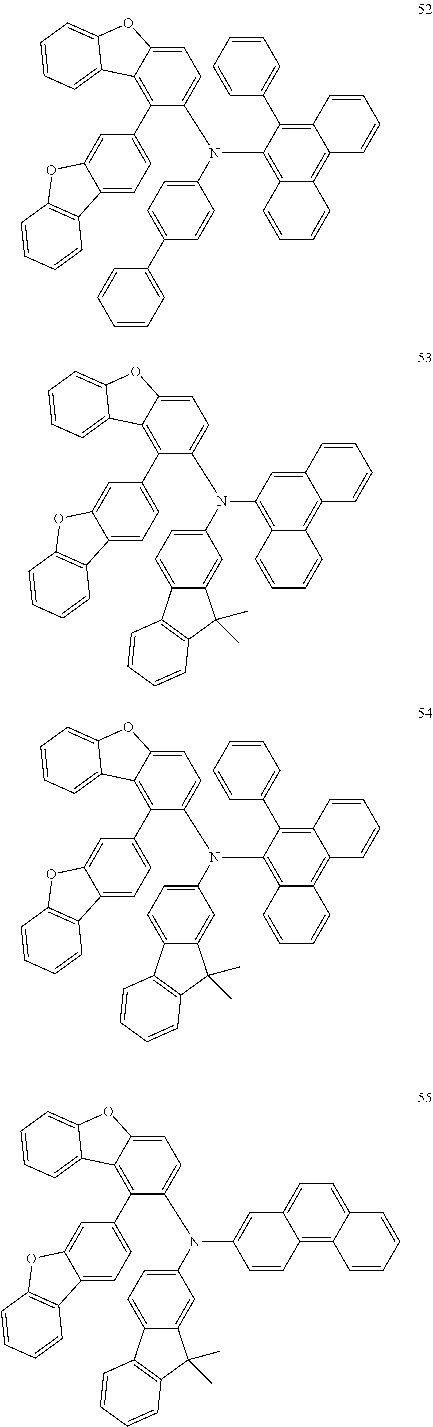

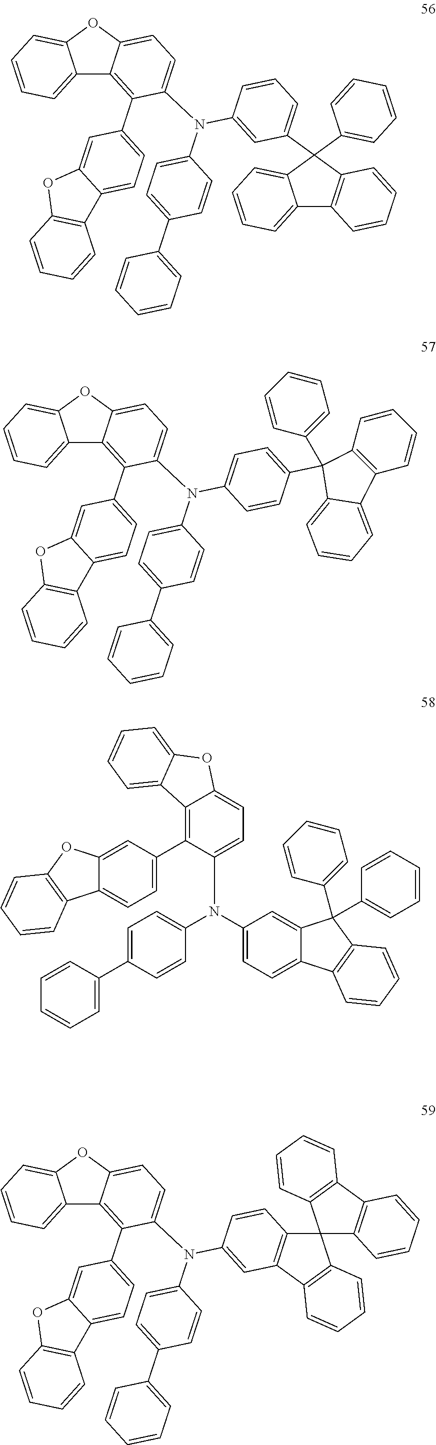 [SMILES: c1ccc(-c2ccc(N(c3cccc(C4(c5ccccc5)c5ccccc5-c5ccccc54)c3)c3ccc4oc5ccccc5c4c3-c3ccc4c(c3)oc3ccccc34)cc2)cc1 ; c1ccc(-c2ccc(N(c3ccc(C4(c5ccccc5)c5ccccc5-c5ccccc54)cc3)c3ccc4oc5ccccc5c4c3-c3ccc4c(c3)oc3ccccc34)cc2)cc1 ; c1ccc(-c2ccc(N(c3ccc4c(c3)C(c3ccccc3)(c3ccccc3)c3ccccc3-4)c3ccc4oc5ccccc5c4c3-c3ccc4c(c3)oc3ccccc34)cc2)cc1 ; c1ccc(-c2ccc(N(c3ccc4c(c3)-c3ccccc3C43c4ccccc4-c4ccccc43)c3ccc4oc5ccccc5c4c3-c3ccc4c(c3)oc3ccccc34)cc2)cc1]

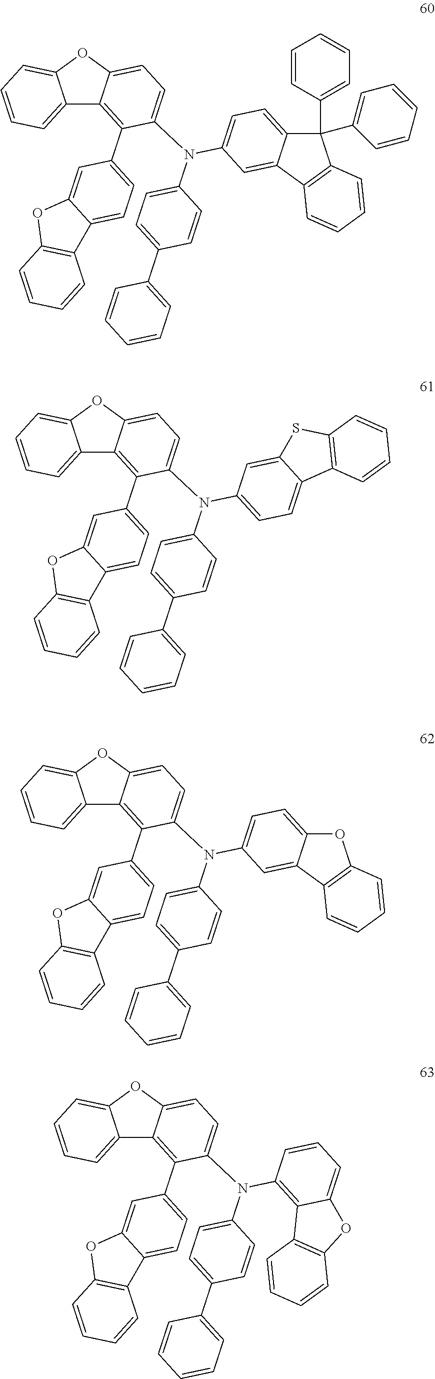

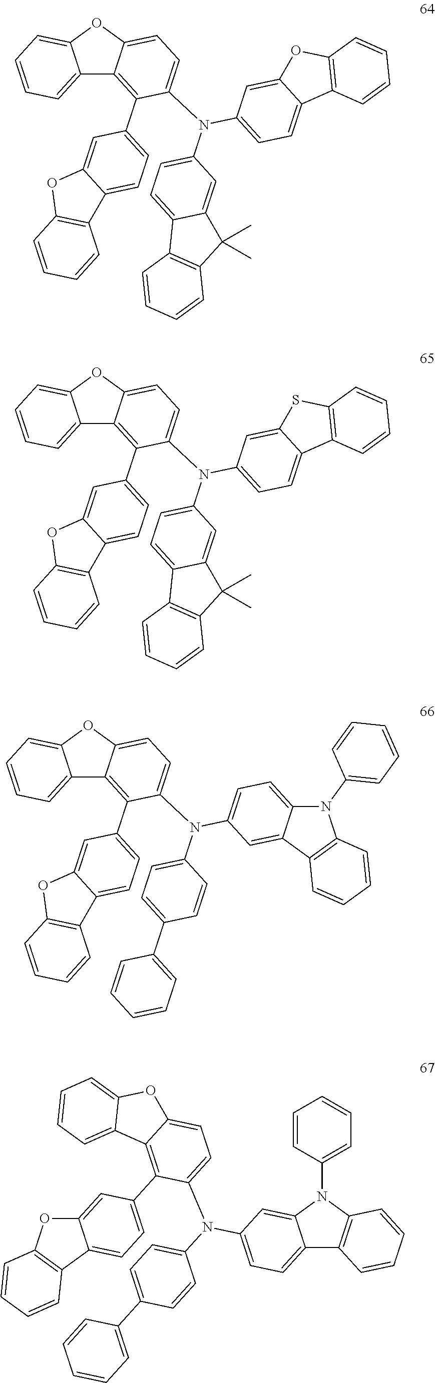 [SMILES: CC1(C)c2ccccc2-c2ccc(N(c3ccc4c(c3)oc3ccccc34)c3ccc4oc5ccccc5c4c3-c3ccc4c(c3)oc3ccccc34)cc21 ; CC1(C)c2ccccc2-c2ccc(N(c3ccc4c(c3)sc3ccccc34)c3ccc4oc5ccccc5c4c3-c3ccc4c(c3)oc3ccccc34)cc21 ; c1ccc(-c2ccc(N(c3ccc4c(c3)c3ccccc3n4-c3ccccc3)c3ccc4oc5ccccc5c4c3-c3ccc4c(c3)oc3ccccc34)cc2)cc1 ; c1ccc(-c2ccc(N(c3ccc4c5ccccc5n(-c5ccccc5)c4c3)c3ccc4oc5ccccc5c4c3-c3ccc4c(c3)oc3ccccc34)cc2)cc1]

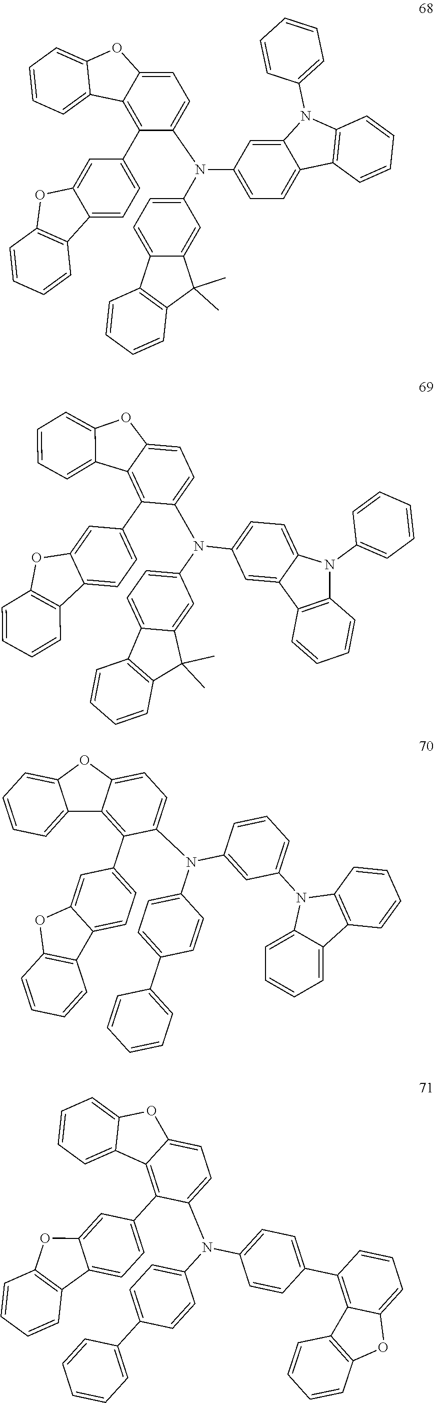 [SMILES: CC1(C)c2ccccc2-c2ccc(N(c3ccc4c5ccccc5n(-c5ccccc5)c4c3)c3ccc4oc5ccccc5c4c3-c3ccc4c(c3)oc3ccccc34)cc21 ; CC1(C)c2ccccc2-c2ccc(N(c3ccc4c(c3)c3ccccc3n4-c3ccccc3)c3ccc4oc5ccccc5c4c3-c3ccc4c(c3)oc3ccccc34)cc21 ; c1ccc(-c2ccc(N(c3cccc(-n4c5ccccc5c5ccccc54)c3)c3ccc4oc5ccccc5c4c3-c3ccc4c(c3)oc3ccccc34)cc2)cc1 ; c1ccc(-c2ccc(N(c3ccc(-c4cccc5oc6ccccc6c45)cc3)c3ccc4oc5ccccc5c4c3-c3ccc4c(c3)oc3ccccc34)cc2)cc1]

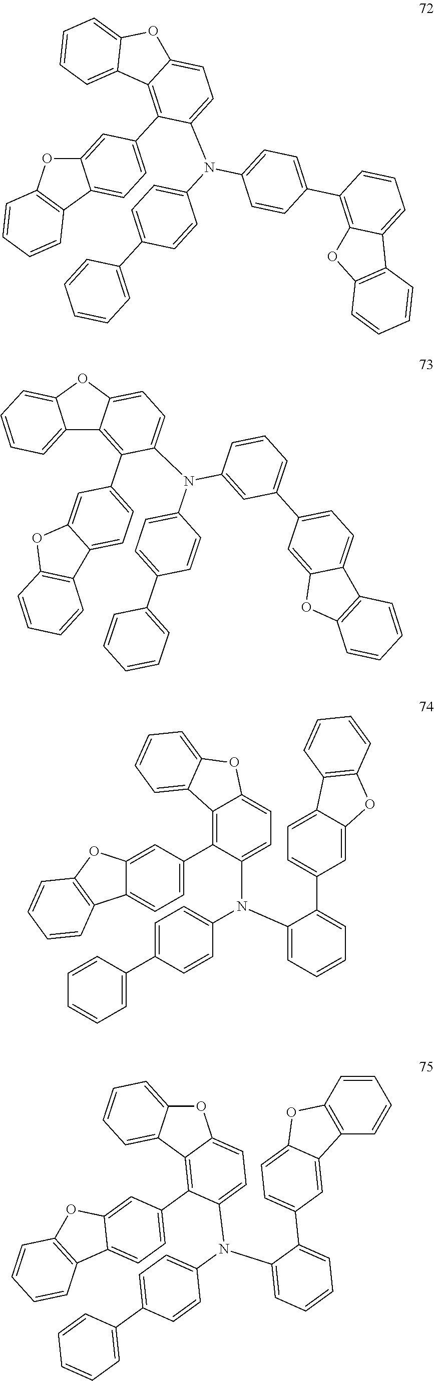 [SMILES: c1ccc(-c2ccc(N(c3ccc(-c4cccc5c4oc4ccccc45)cc3)c3ccc4oc5ccccc5c4c3-c3ccc4c(c3)oc3ccccc34)cc2)cc1 ; c1ccc(-c2ccc(N(c3cccc(-c4ccc5c(c4)oc4ccccc45)c3)c3ccc4oc5ccccc5c4c3-c3ccc4c(c3)oc3ccccc34)cc2)cc1 ; c1ccc(-c2ccc(N(c3ccccc3-c3ccc4c(c3)oc3ccccc34)c3ccc4oc5ccccc5c4c3-c3ccc4c(c3)oc3ccccc34)cc2)cc1 ; c1ccc(-c2ccc(N(c3ccccc3-c3ccc4oc5ccccc5c4c3)c3ccc4oc5ccccc5c4c3-c3ccc4c(c3)oc3ccccc34)cc2)cc1]

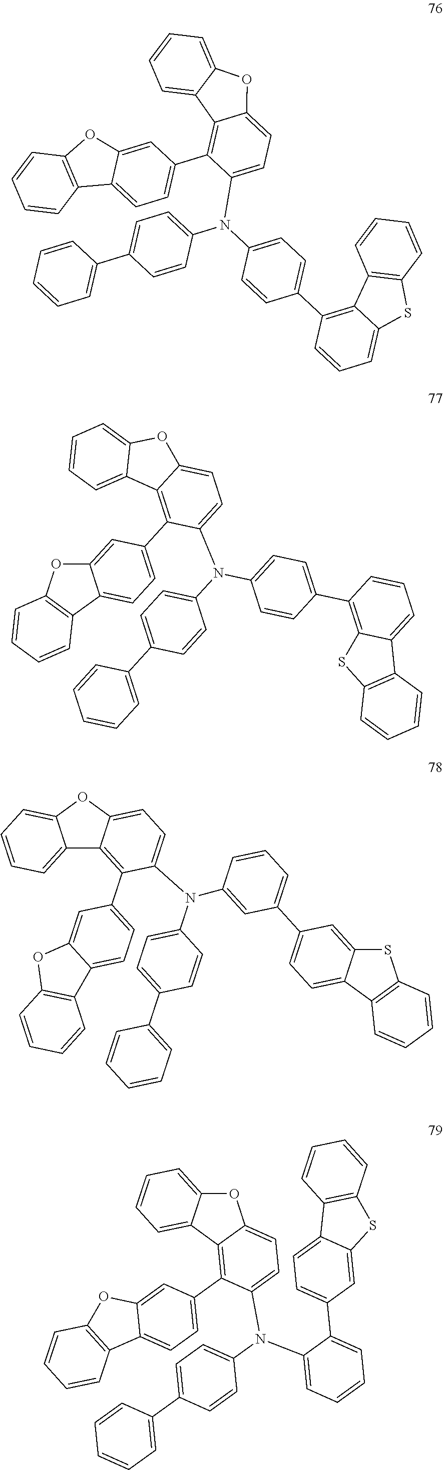 [SMILES: c1ccc(-c2ccc(N(c3ccc(-c4cccc5sc6ccccc6c45)cc3)c3ccc4oc5ccccc5c4c3-c3ccc4c(c3)oc3ccccc34)cc2)cc1 ; c1ccc(-c2ccc(N(c3ccc(-c4cccc5c4sc4ccccc45)cc3)c3ccc4oc5ccccc5c4c3-c3ccc4c(c3)oc3ccccc34)cc2)cc1 ; c1ccc(-c2ccc(N(c3cccc(-c4ccc5c(c4)sc4ccccc45)c3)c3ccc4oc5ccccc5c4c3-c3ccc4c(c3)oc3ccccc34)cc2)cc1 ; c1ccc(-c2ccc(N(c3ccccc3-c3ccc4c(c3)sc3ccccc34)c3ccc4oc5ccccc5c4c3-c3ccc4c(c3)oc3ccccc34)cc2)cc1]

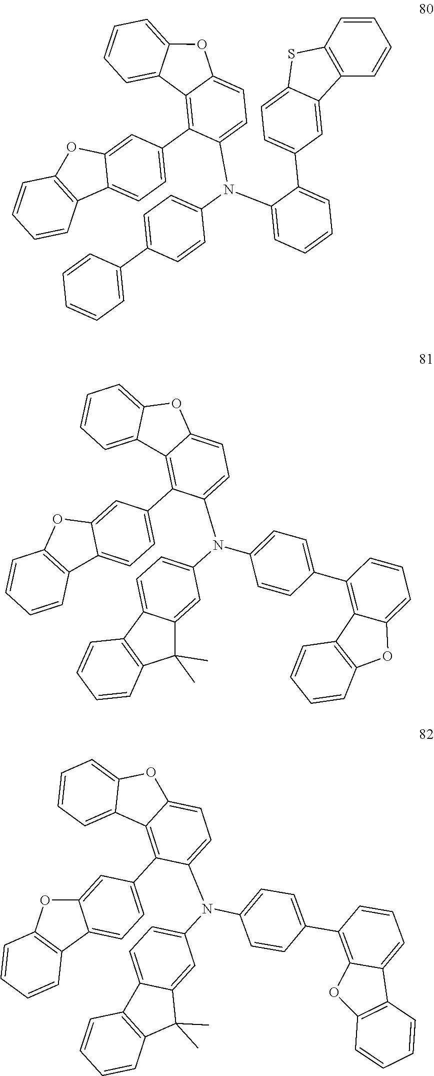 [SMILES: c1ccc(-c2ccc(N(c3ccccc3-c3ccc4sc5ccccc5c4c3)c3ccc4oc5ccccc5c4c3-c3ccc4c(c3)oc3ccccc34)cc2)cc1 ; CC1(C)c2ccccc2-c2ccc(N(c3ccc(-c4cccc5oc6ccccc6c45)cc3)c3ccc4oc5ccccc5c4c3-c3ccc4c(c3)oc3ccccc34)cc21 ; CC1(C)c2ccccc2-c2ccc(N(c3ccc(-c4cccc5c4oc4ccccc45)cc3)c3ccc4oc5ccccc5c4c3-c3ccc4c(c3)oc3ccccc34)cc21]

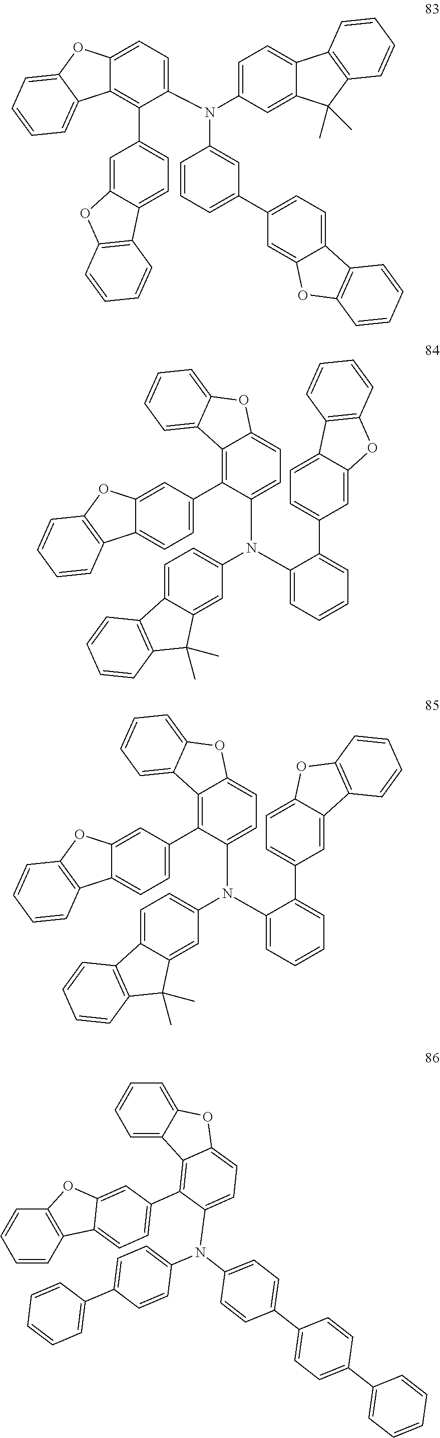

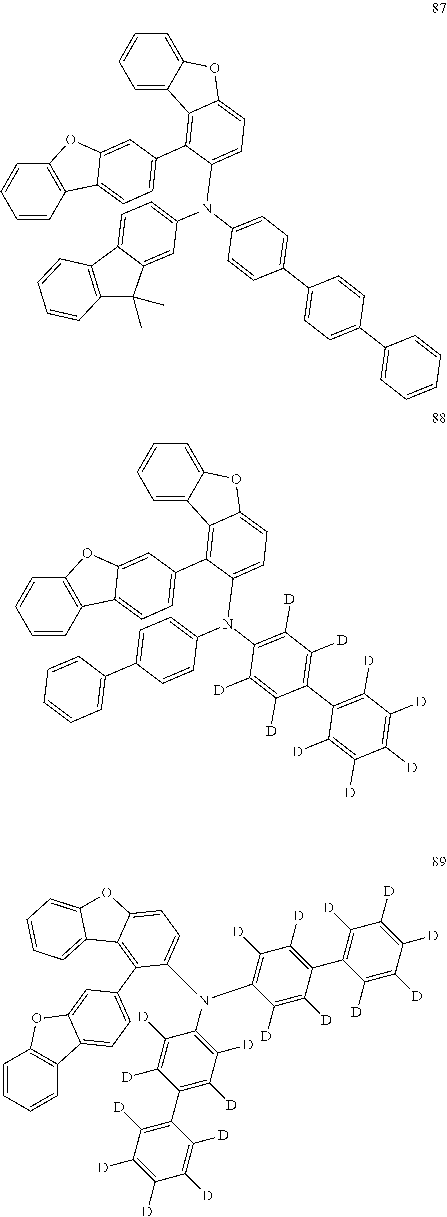 [SMILES: CC1(C)c2ccccc2-c2ccc(N(c3ccc(-c4ccc(-c5ccccc5)cc4)cc3)c3ccc4oc5ccccc5c4c3-c3ccc4c(c3)oc3ccccc34)cc21 ; [2H]c1c([2H])c([2H])c(-c2c([2H])c([2H])c(N(c3ccc(-c4ccccc4)cc3)c3ccc4oc5ccccc5c4c3-c3ccc4c(c3)oc3ccccc34)c([2H])c2[2H])c([2H])c1[2H] ; [2H]c1c([2H])c([2H])c(-c2c([2H])c([2H])c(N(c3ccc4oc5ccccc5c4c3-c3ccc4c(c3)oc3ccccc34)c3c([2H])c([2H])c(-c4c([2H])c([2H])c([2H])c([2H])c4[2H])c([2H])c3[2H])c([2H])c2[2H])c([2H])c1[2H]]

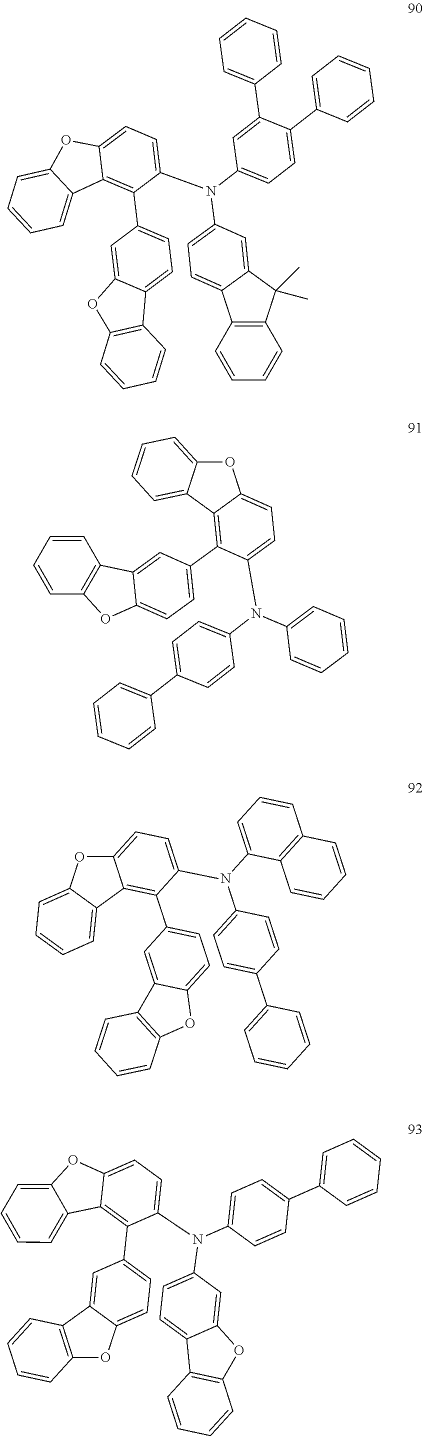 [SMILES: CC1(C)c2ccccc2-c2ccc(N(c3ccc(-c4ccccc4)c(-c4ccccc4)c3)c3ccc4oc5ccccc5c4c3-c3ccc4c(c3)oc3ccccc34)cc21 ; c1ccc(-c2ccc(N(c3ccccc3)c3ccc4oc5ccccc5c4c3-c3ccc4oc5ccccc5c4c3)cc2)cc1 ; c1ccc(-c2ccc(N(c3ccc4oc5ccccc5c4c3-c3ccc4oc5ccccc5c4c3)c3cccc4ccccc34)cc2)cc1 ; c1ccc(-c2ccc(N(c3ccc4c(c3)oc3ccccc34)c3ccc4oc5ccccc5c4c3-c3ccc4oc5ccccc5c4c3)cc2)cc1]

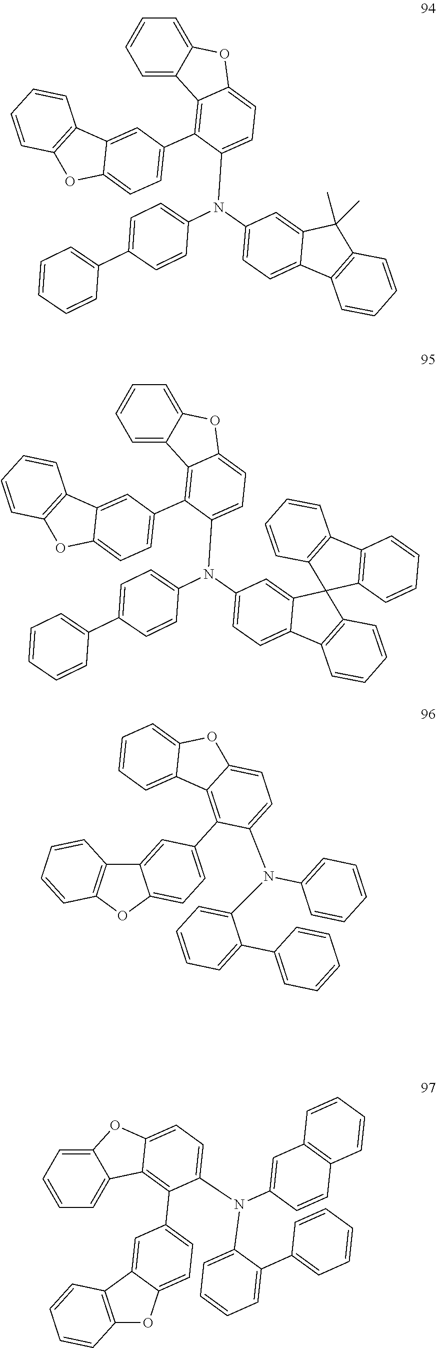 [SMILES: CC1(C)c2ccccc2-c2ccc(N(c3ccc(-c4ccccc4)cc3)c3ccc4oc5ccccc5c4c3-c3ccc4oc5ccccc5c4c3)cc21 ; c1ccc(-c2ccc(N(c3ccc4c(c3)C3(c5ccccc5-c5ccccc53)c3ccccc3-4)c3ccc4oc5ccccc5c4c3-c3ccc4oc5ccccc5c4c3)cc2)cc1 ; c1ccc(-c2ccccc2N(c2ccccc2)c2ccc3oc4ccccc4c3c2-c2ccc3oc4ccccc4c3c2)cc1 ; c1ccc(-c2ccccc2N(c2ccc3ccccc3c2)c2ccc3oc4ccccc4c3c2-c2ccc3oc4ccccc4c3c2)cc1]

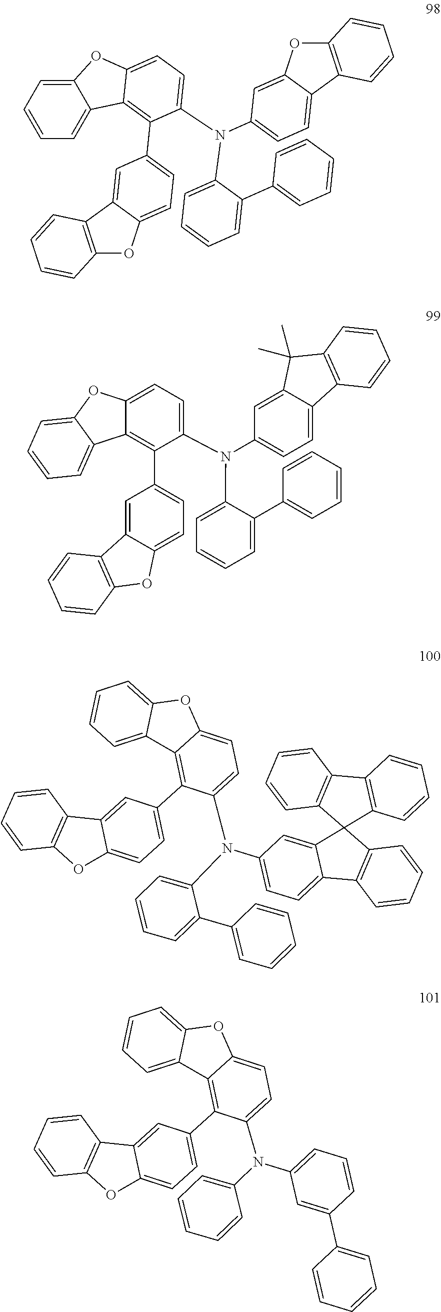 [SMILES: c1ccc(-c2ccccc2N(c2ccc3c(c2)oc2ccccc23)c2ccc3oc4ccccc4c3c2-c2ccc3oc4ccccc4c3c2)cc1 ; CC1(C)c2ccccc2-c2ccc(N(c3ccccc3-c3ccccc3)c3ccc4oc5ccccc5c4c3-c3ccc4oc5ccccc5c4c3)cc21 ; c1ccc(-c2ccccc2N(c2ccc3c(c2)C2(c4ccccc4-c4ccccc42)c2ccccc2-3)c2ccc3oc4ccccc4c3c2-c2ccc3oc4ccccc4c3c2)cc1 ; c1ccc(-c2cccc(N(c3ccccc3)c3ccc4oc5ccccc5c4c3-c3ccc4oc5ccccc5c4c3)c2)cc1]

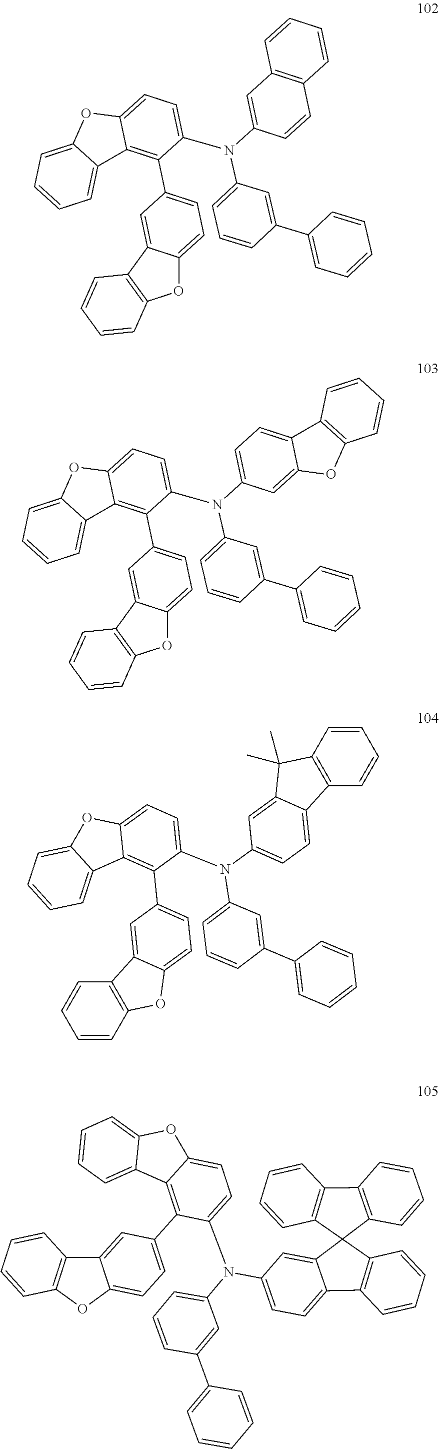 [SMILES: c1ccc(-c2cccc(N(c3ccc4ccccc4c3)c3ccc4oc5ccccc5c4c3-c3ccc4oc5ccccc5c4c3)c2)cc1 ; c1ccc(-c2cccc(N(c3ccc4c(c3)oc3ccccc34)c3ccc4oc5ccccc5c4c3-c3ccc4oc5ccccc5c4c3)c2)cc1 ; CC1(C)c2ccccc2-c2ccc(N(c3cccc(-c4ccccc4)c3)c3ccc4oc5ccccc5c4c3-c3ccc4oc5ccccc5c4c3)cc21 ; c1ccc(-c2cccc(N(c3ccc4c(c3)C3(c5ccccc5-c5ccccc53)c3ccccc3-4)c3ccc4oc5ccccc5c4c3-c3ccc4oc5ccccc5c4c3)c2)cc1]

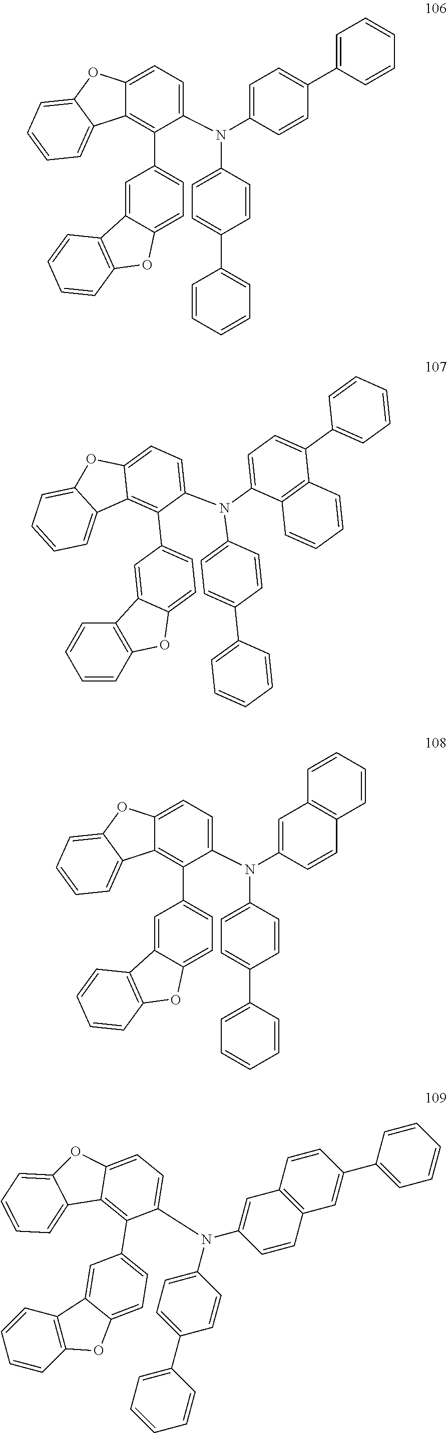 [SMILES: c1ccc(-c2ccc(N(c3ccc(-c4ccccc4)cc3)c3ccc4oc5ccccc5c4c3-c3ccc4oc5ccccc5c4c3)cc2)cc1 ; c1ccc(-c2ccc(N(c3ccc4oc5ccccc5c4c3-c3ccc4oc5ccccc5c4c3)c3ccc(-c4ccccc4)c4ccccc34)cc2)cc1 ; c1ccc(-c2ccc(N(c3ccc4ccccc4c3)c3ccc4oc5ccccc5c4c3-c3ccc4oc5ccccc5c4c3)cc2)cc1 ; c1ccc(-c2ccc(N(c3ccc4cc(-c5ccccc5)ccc4c3)c3ccc4oc5ccccc5c4c3-c3ccc4oc5ccccc5c4c3)cc2)cc1]

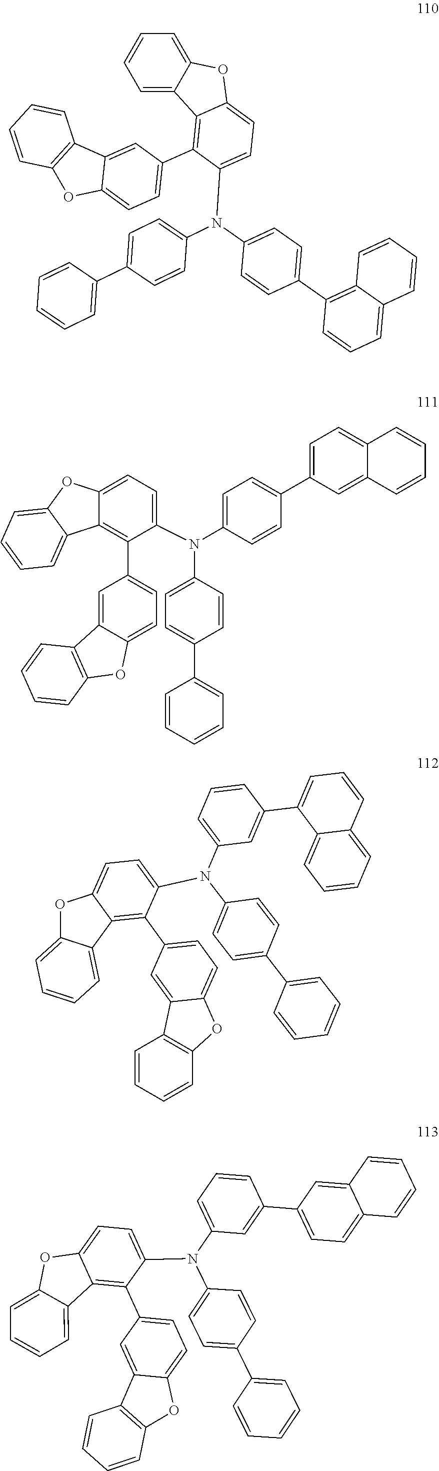 [SMILES: c1ccc(-c2ccc(N(c3ccc(-c4cccc5ccccc45)cc3)c3ccc4oc5ccccc5c4c3-c3ccc4oc5ccccc5c4c3)cc2)cc1 ; c1ccc(-c2ccc(N(c3ccc(-c4ccc5ccccc5c4)cc3)c3ccc4oc5ccccc5c4c3-c3ccc4oc5ccccc5c4c3)cc2)cc1 ; c1ccc(-c2ccc(N(c3cccc(-c4cccc5ccccc45)c3)c3ccc4oc5ccccc5c4c3-c3ccc4oc5ccccc5c4c3)cc2)cc1 ; c1ccc(-c2ccc(N(c3cccc(-c4ccc5ccccc5c4)c3)c3ccc4oc5ccccc5c4c3-c3ccc4oc5ccccc5c4c3)cc2)cc1]

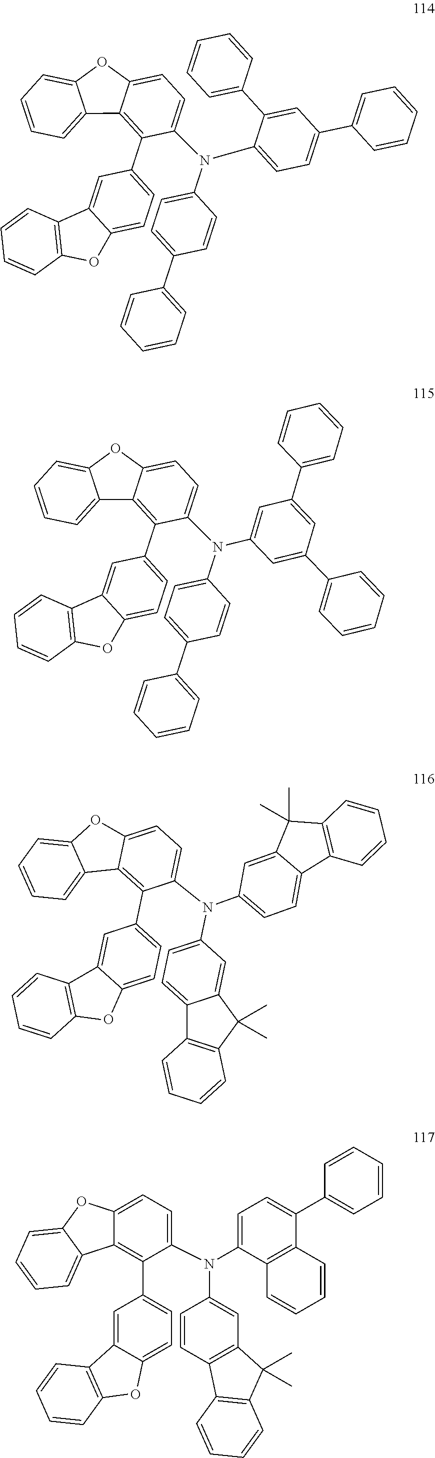 [SMILES: c1ccc(-c2ccc(N(c3ccc(-c4ccccc4)cc3-c3ccccc3)c3ccc4oc5ccccc5c4c3-c3ccc4oc5ccccc5c4c3)cc2)cc1 ; c1ccc(-c2ccc(N(c3cc(-c4ccccc4)cc(-c4ccccc4)c3)c3ccc4oc5ccccc5c4c3-c3ccc4oc5ccccc5c4c3)cc2)cc1 ; CC1(C)c2ccccc2-c2ccc(N(c3ccc4c(c3)C(C)(C)c3ccccc3-4)c3ccc4oc5ccccc5c4c3-c3ccc4oc5ccccc5c4c3)cc21 ; CC1(C)c2ccccc2-c2ccc(N(c3ccc4oc5ccccc5c4c3-c3ccc4oc5ccccc5c4c3)c3ccc(-c4ccccc4)c4ccccc34)cc21]

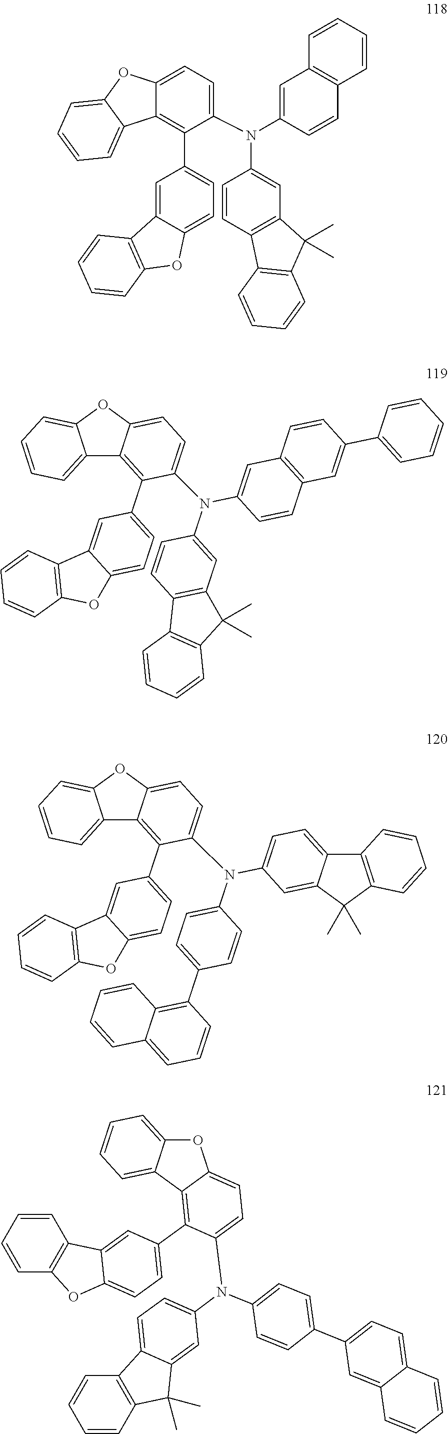 [SMILES: CC1(C)c2ccccc2-c2ccc(N(c3ccc4ccccc4c3)c3ccc4oc5ccccc5c4c3-c3ccc4oc5ccccc5c4c3)cc21 ; CC1(C)c2ccccc2-c2ccc(N(c3ccc4cc(-c5ccccc5)ccc4c3)c3ccc4oc5ccccc5c4c3-c3ccc4oc5ccccc5c4c3)cc21 ; CC1(C)c2ccccc2-c2ccc(N(c3ccc(-c4cccc5ccccc45)cc3)c3ccc4oc5ccccc5c4c3-c3ccc4oc5ccccc5c4c3)cc21 ; CC1(C)c2ccccc2-c2ccc(N(c3ccc(-c4ccc5ccccc5c4)cc3)c3ccc4oc5ccccc5c4c3-c3ccc4oc5ccccc5c4c3)cc21]

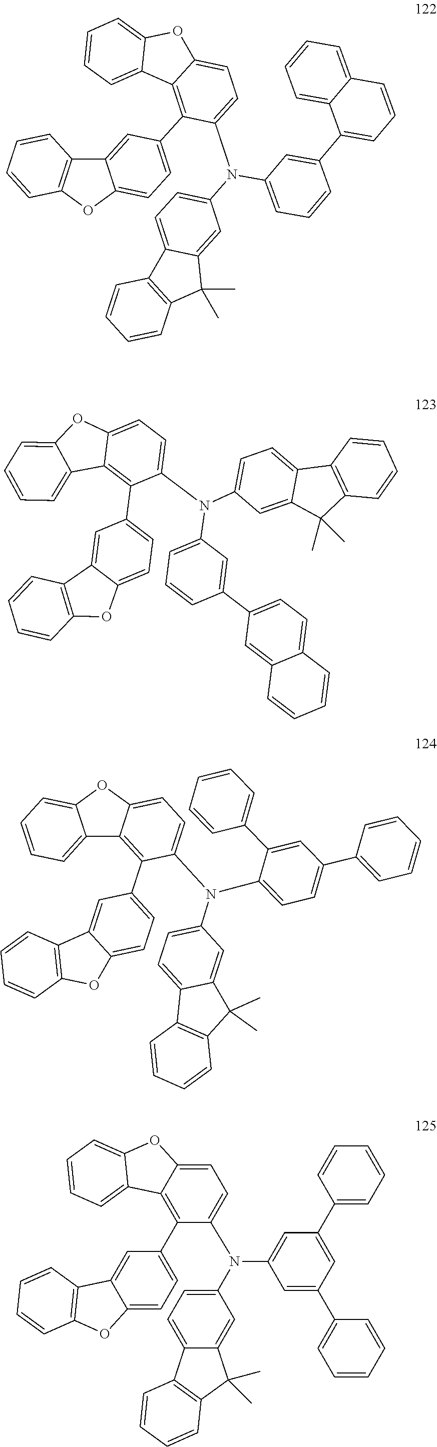 [SMILES: CC1(C)c2ccccc2-c2ccc(N(c3cccc(-c4cccc5ccccc45)c3)c3ccc4oc5ccccc5c4c3-c3ccc4oc5ccccc5c4c3)cc21 ; CC1(C)c2ccccc2-c2ccc(N(c3cccc(-c4ccc5ccccc5c4)c3)c3ccc4oc5ccccc5c4c3-c3ccc4oc5ccccc5c4c3)cc21 ; CC1(C)c2ccccc2-c2ccc(N(c3ccc(-c4ccccc4)cc3-c3ccccc3)c3ccc4oc5ccccc5c4c3-c3ccc4oc5ccccc5c4c3)cc21 ; CC1(C)c2ccccc2-c2ccc(N(c3cc(-c4ccccc4)cc(-c4ccccc4)c3)c3ccc4oc5ccccc5c4c3-c3ccc4oc5ccccc5c4c3)cc21]

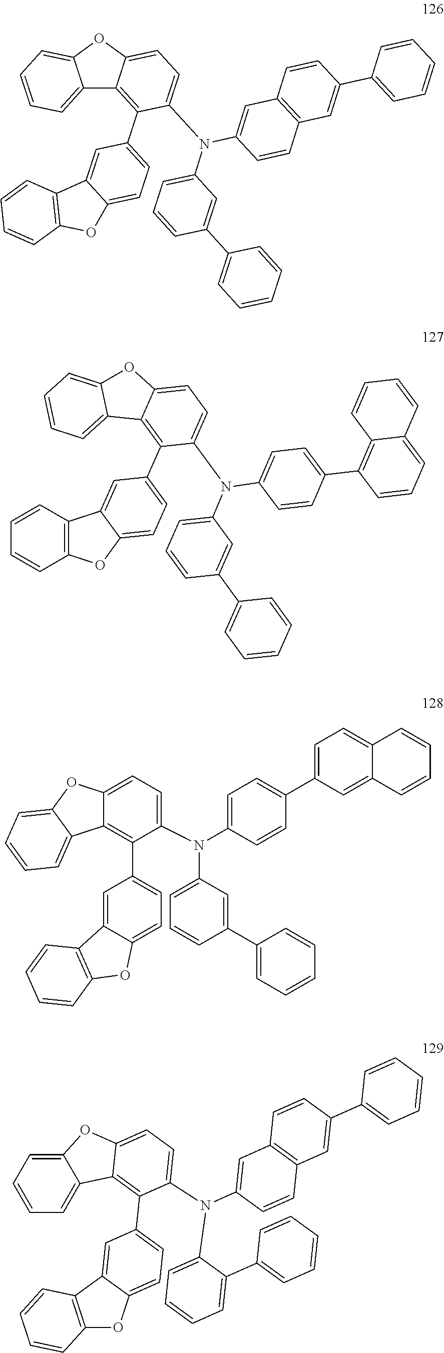 [SMILES: c1ccc(-c2cccc(N(c3ccc4cc(-c5ccccc5)ccc4c3)c3ccc4oc5ccccc5c4c3-c3ccc4oc5ccccc5c4c3)c2)cc1 ; c1ccc(-c2cccc(N(c3ccc(-c4cccc5ccccc45)cc3)c3ccc4oc5ccccc5c4c3-c3ccc4oc5ccccc5c4c3)c2)cc1 ; c1ccc(-c2cccc(N(c3ccc(-c4ccc5ccccc5c4)cc3)c3ccc4oc5ccccc5c4c3-c3ccc4oc5ccccc5c4c3)c2)cc1 ; c1ccc(-c2ccc3cc(N(c4ccccc4-c4ccccc4)c4ccc5oc6ccccc6c5c4-c4ccc5oc6ccccc6c5c4)ccc3c2)cc1]

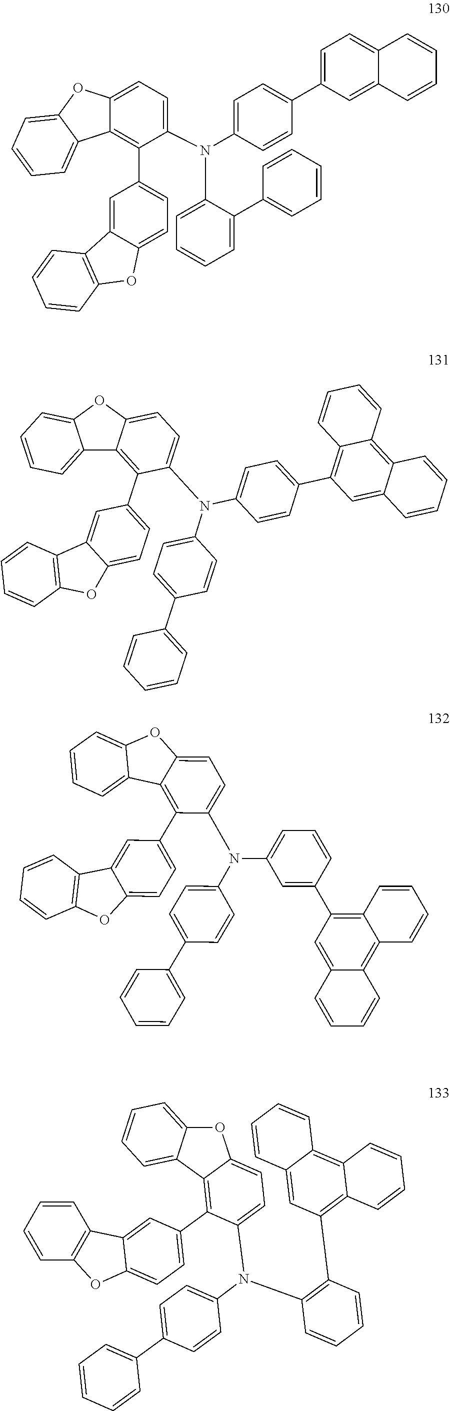

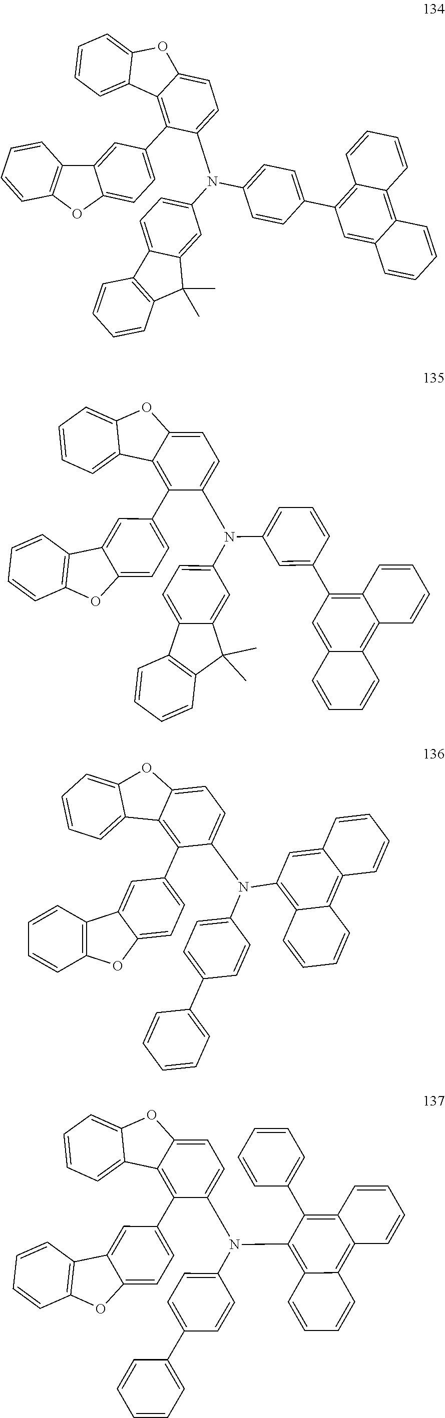

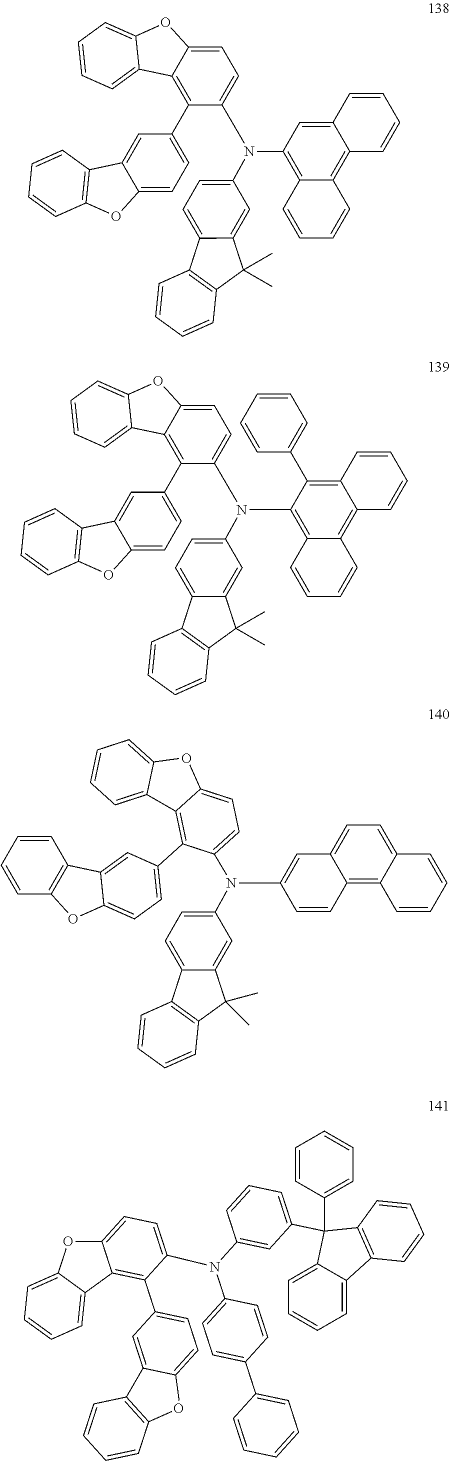 [SMILES: CC1(C)c2ccccc2-c2ccc(N(c3ccc4oc5ccccc5c4c3-c3ccc4oc5ccccc5c4c3)c3cc4ccccc4c4ccccc34)cc21 ; CC1(C)c2ccccc2-c2ccc(N(c3ccc4oc5ccccc5c4c3-c3ccc4oc5ccccc5c4c3)c3c(-c4ccccc4)c4ccccc4c4ccccc34)cc21 ; CC1(C)c2ccccc2-c2ccc(N(c3ccc4c(ccc5ccccc54)c3)c3ccc4oc5ccccc5c4c3-c3ccc4oc5ccccc5c4c3)cc21 ; c1ccc(-c2ccc(N(c3cccc(C4(c5ccccc5)c5ccccc5-c5ccccc54)c3)c3ccc4oc5ccccc5c4c3-c3ccc4oc5ccccc5c4c3)cc2)cc1]

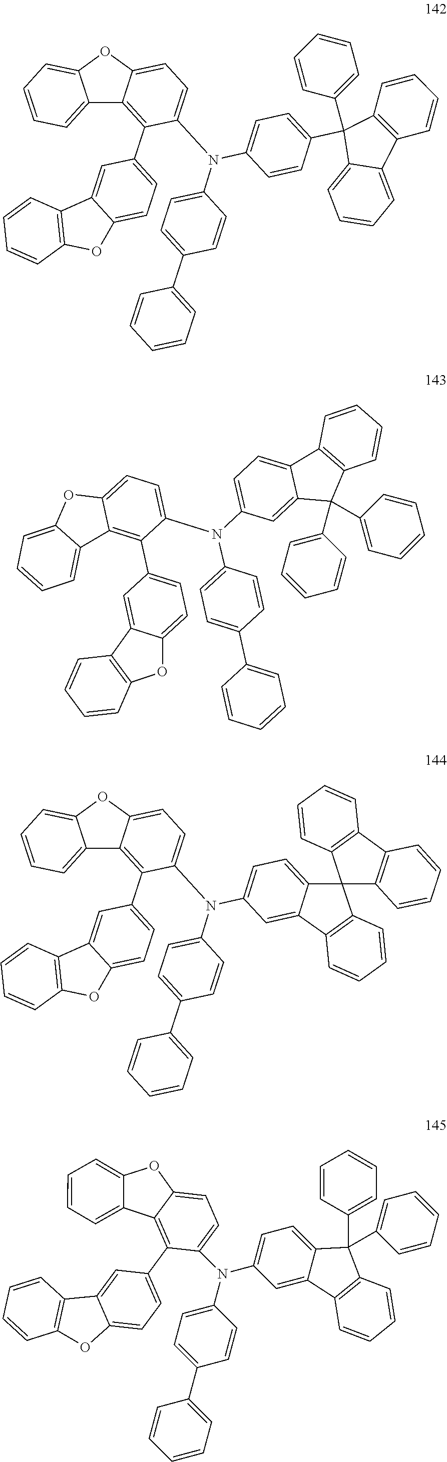 [SMILES: c1ccc(-c2ccc(N(c3ccc(C4(c5ccccc5)c5ccccc5-c5ccccc54)cc3)c3ccc4oc5ccccc5c4c3-c3ccc4oc5ccccc5c4c3)cc2)cc1 ; c1ccc(-c2ccc(N(c3ccc4c(c3)C(c3ccccc3)(c3ccccc3)c3ccccc3-4)c3ccc4oc5ccccc5c4c3-c3ccc4oc5ccccc5c4c3)cc2)cc1 ; c1ccc(-c2ccc(N(c3ccc4c(c3)-c3ccccc3C43c4ccccc4-c4ccccc43)c3ccc4oc5ccccc5c4c3-c3ccc4oc5ccccc5c4c3)cc2)cc1 ; c1ccc(-c2ccc(N(c3ccc4c(c3)-c3ccccc3C4(c3ccccc3)c3ccccc3)c3ccc4oc5ccccc5c4c3-c3ccc4oc5ccccc5c4c3)cc2)cc1]

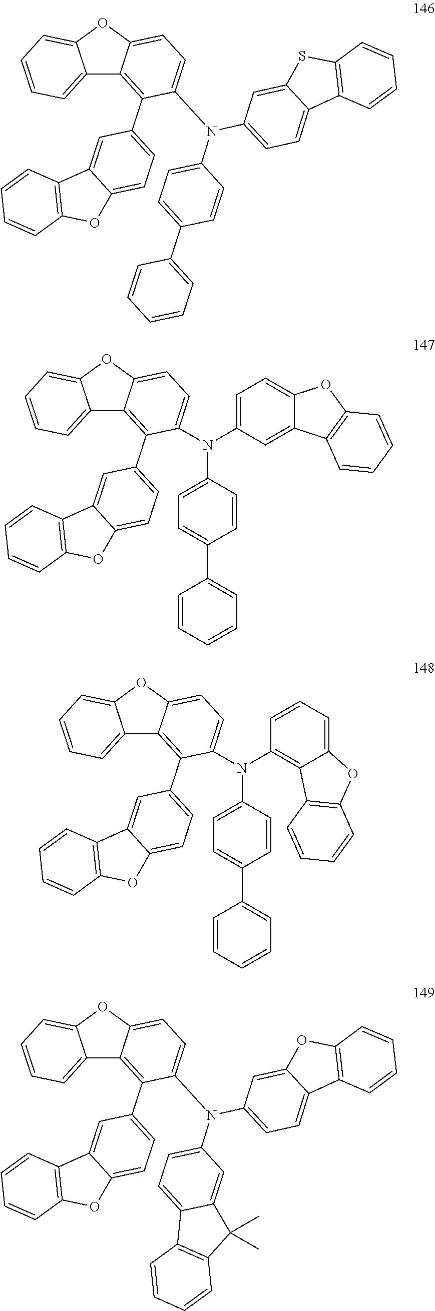 [SMILES: c1ccc(-c2ccc(N(c3ccc4c(c3)sc3ccccc34)c3ccc4oc5ccccc5c4c3-c3ccc4oc5ccccc5c4c3)cc2)cc1 ; c1ccc(-c2ccc(N(c3ccc4oc5ccccc5c4c3)c3ccc4oc5ccccc5c4c3-c3ccc4oc5ccccc5c4c3)cc2)cc1 ; c1ccc(-c2ccc(N(c3ccc4oc5ccccc5c4c3-c3ccc4oc5ccccc5c4c3)c3cccc4oc5ccccc5c34)cc2)cc1 ; CC1(C)c2ccccc2-c2ccc(N(c3ccc4c(c3)oc3ccccc34)c3ccc4oc5ccccc5c4c3-c3ccc4oc5ccccc5c4c3)cc21]

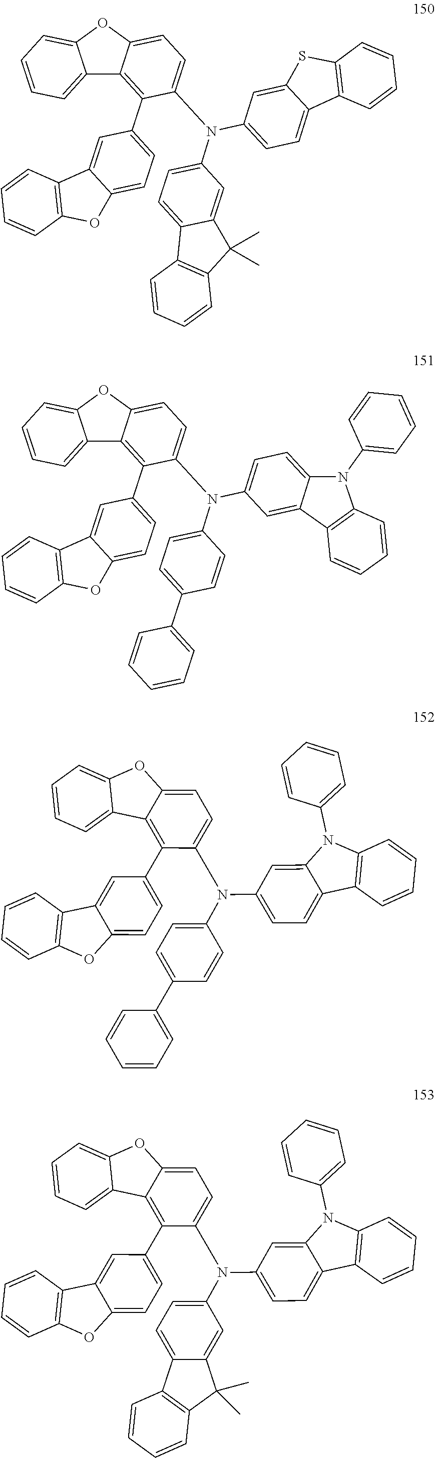 [SMILES: CC1(C)c2ccccc2-c2ccc(N(c3ccc4c(c3)sc3ccccc34)c3ccc4oc5ccccc5c4c3-c3ccc4oc5ccccc5c4c3)cc21 ; c1ccc(-c2ccc(N(c3ccc4c(c3)c3ccccc3n4-c3ccccc3)c3ccc4oc5ccccc5c4c3-c3ccc4oc5ccccc5c4c3)cc2)cc1 ; c1ccc(-c2ccc(N(c3ccc4c5ccccc5n(-c5ccccc5)c4c3)c3ccc4oc5ccccc5c4c3-c3ccc4oc5ccccc5c4c3)cc2)cc1 ; CC1(C)c2ccccc2-c2ccc(N(c3ccc4c5ccccc5n(-c5ccccc5)c4c3)c3ccc4oc5ccccc5c4c3-c3ccc4oc5ccccc5c4c3)cc21]

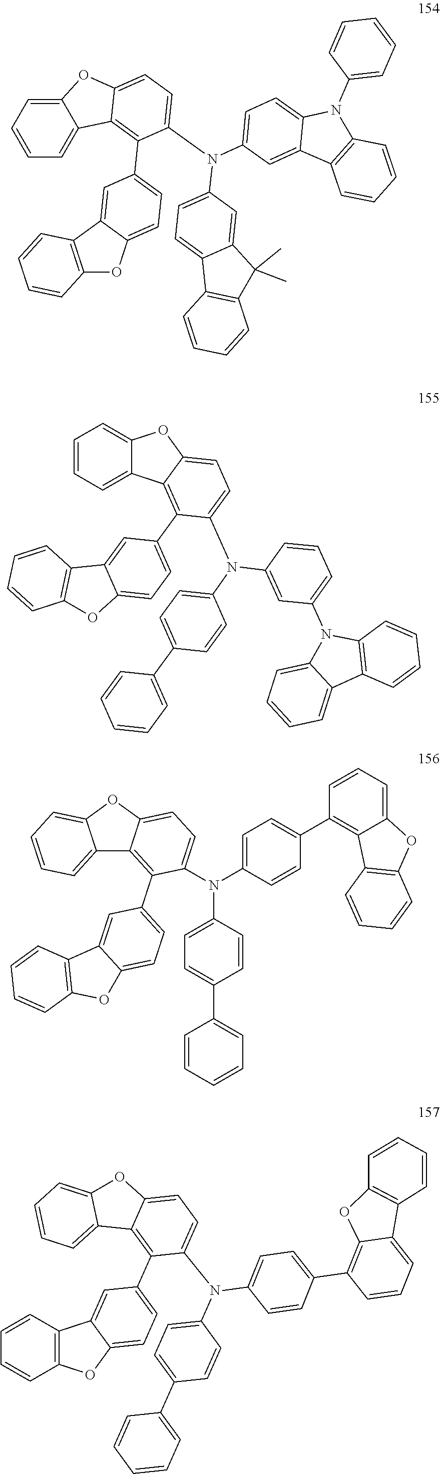 [SMILES: CC1(C)c2ccccc2-c2ccc(N(c3ccc4c(c3)c3ccccc3n4-c3ccccc3)c3ccc4oc5ccccc5c4c3-c3ccc4oc5ccccc5c4c3)cc21 ; c1ccc(-c2ccc(N(c3cccc(-n4c5ccccc5c5ccccc54)c3)c3ccc4oc5ccccc5c4c3-c3ccc4oc5ccccc5c4c3)cc2)cc1 ; c1ccc(-c2ccc(N(c3ccc(-c4cccc5oc6ccccc6c45)cc3)c3ccc4oc5ccccc5c4c3-c3ccc4oc5ccccc5c4c3)cc2)cc1 ; c1ccc(-c2ccc(N(c3ccc(-c4cccc5c4oc4ccccc45)cc3)c3ccc4oc5ccccc5c4c3-c3ccc4oc5ccccc5c4c3)cc2)cc1]

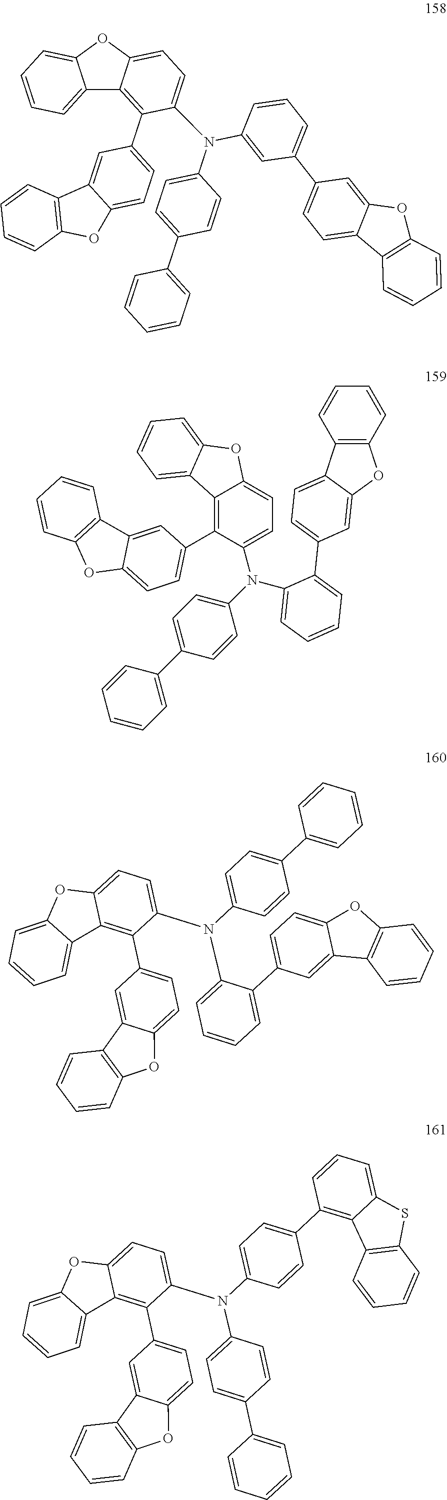 [SMILES: c1ccc(-c2ccc(N(c3cccc(-c4ccc5c(c4)oc4ccccc45)c3)c3ccc4oc5ccccc5c4c3-c3ccc4oc5ccccc5c4c3)cc2)cc1 ; c1ccc(-c2ccc(N(c3ccccc3-c3ccc4c(c3)oc3ccccc34)c3ccc4oc5ccccc5c4c3-c3ccc4oc5ccccc5c4c3)cc2)cc1 ; c1ccc(-c2ccc(N(c3ccccc3-c3ccc4oc5ccccc5c4c3)c3ccc4oc5ccccc5c4c3-c3ccc4oc5ccccc5c4c3)cc2)cc1 ; c1ccc(-c2ccc(N(c3ccc(-c4cccc5sc6ccccc6c45)cc3)c3ccc4oc5ccccc5c4c3-c3ccc4oc5ccccc5c4c3)cc2)cc1]

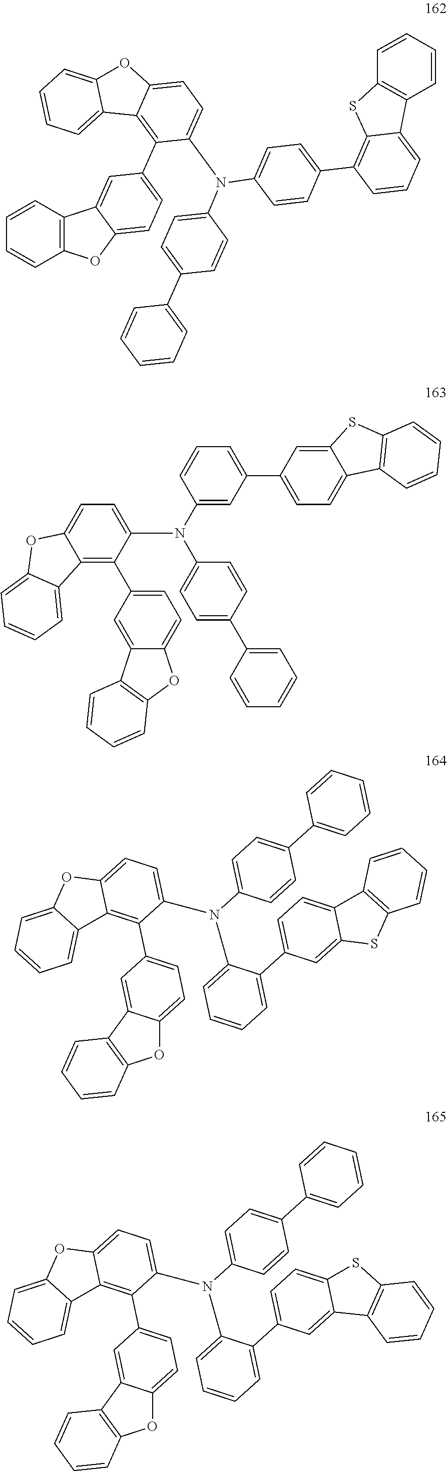 [SMILES: c1ccc(-c2ccc(N(c3ccc(-c4cccc5c4sc4ccccc45)cc3)c3ccc4oc5ccccc5c4c3-c3ccc4oc5ccccc5c4c3)cc2)cc1 ; c1ccc(-c2ccc(N(c3cccc(-c4ccc5c(c4)sc4ccccc45)c3)c3ccc4oc5ccccc5c4c3-c3ccc4oc5ccccc5c4c3)cc2)cc1 ; c1ccc(-c2ccc(N(c3ccccc3-c3ccc4c(c3)sc3ccccc34)c3ccc4oc5ccccc5c4c3-c3ccc4oc5ccccc5c4c3)cc2)cc1 ; c1ccc(-c2ccc(N(c3ccccc3-c3ccc4sc5ccccc5c4c3)c3ccc4oc5ccccc5c4c3-c3ccc4oc5ccccc5c4c3)cc2)cc1]

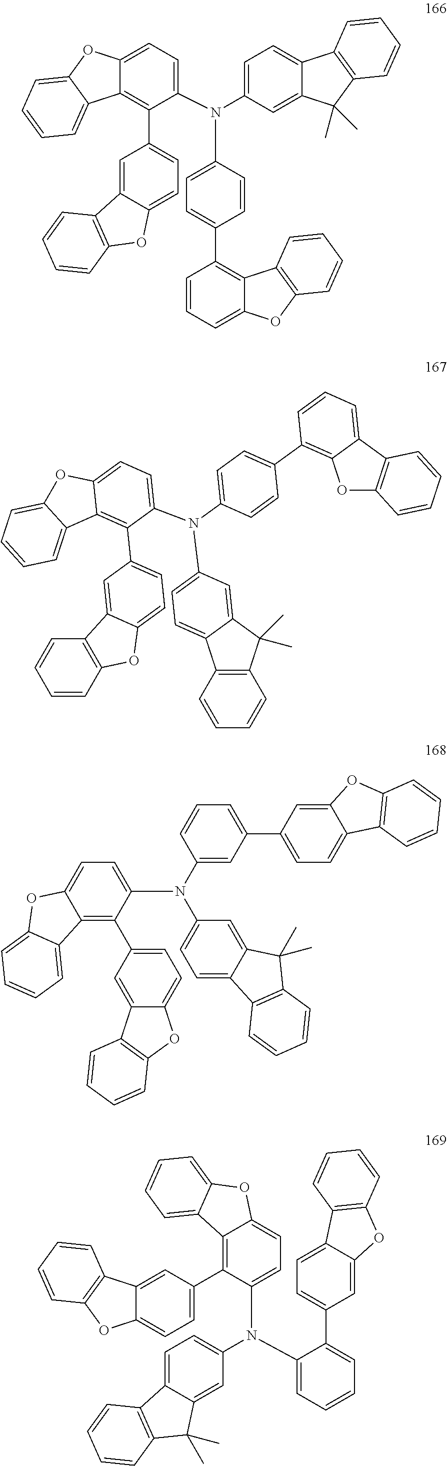 [SMILES: CC1(C)c2ccccc2-c2ccc(N(c3ccc(-c4cccc5oc6ccccc6c45)cc3)c3ccc4oc5ccccc5c4c3-c3ccc4oc5ccccc5c4c3)cc21 ; CC1(C)c2ccccc2-c2ccc(N(c3ccc(-c4cccc5c4oc4ccccc45)cc3)c3ccc4oc5ccccc5c4c3-c3ccc4oc5ccccc5c4c3)cc21 ; CC1(C)c2ccccc2-c2ccc(N(c3cccc(-c4ccc5c(c4)oc4ccccc45)c3)c3ccc4oc5ccccc5c4c3-c3ccc4oc5ccccc5c4c3)cc21 ; CC1(C)c2ccccc2-c2ccc(N(c3ccccc3-c3ccc4c(c3)oc3ccccc34)c3ccc4oc5ccccc5c4c3-c3ccc4oc5ccccc5c4c3)cc21]

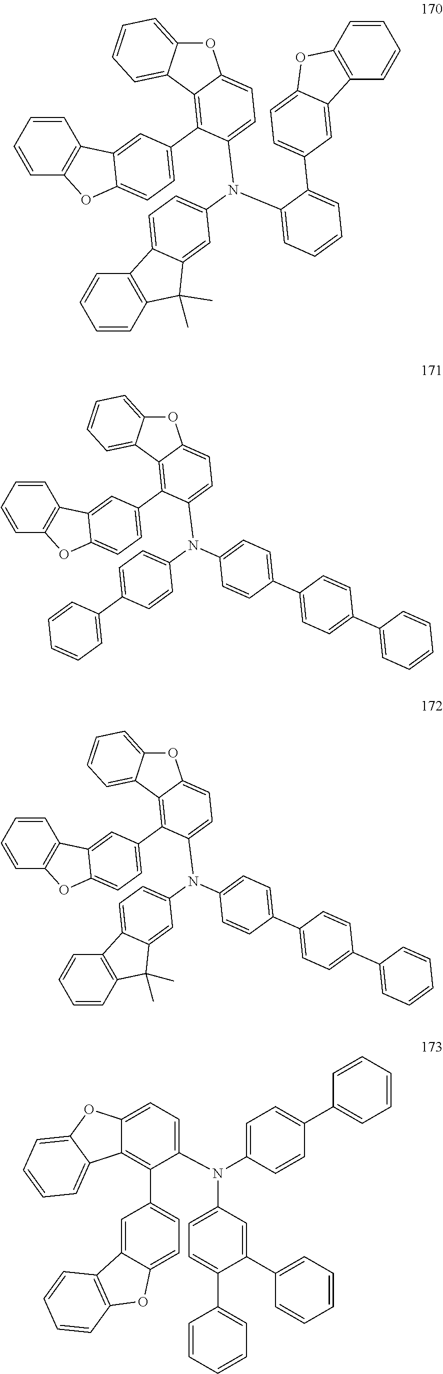 [SMILES: CC1(C)c2ccccc2-c2ccc(N(c3ccccc3-c3ccc4oc5ccccc5c4c3)c3ccc4oc5ccccc5c4c3-c3ccc4oc5ccccc5c4c3)cc21 ; c1ccc(-c2ccc(-c3ccc(N(c4ccc(-c5ccccc5)cc4)c4ccc5oc6ccccc6c5c4-c4ccc5oc6ccccc6c5c4)cc3)cc2)cc1 ; CC1(C)c2ccccc2-c2ccc(N(c3ccc(-c4ccc(-c5ccccc5)cc4)cc3)c3ccc4oc5ccccc5c4c3-c3ccc4oc5ccccc5c4c3)cc21 ; c1ccc(-c2ccc(N(c3ccc(-c4ccccc4)c(-c4ccccc4)c3)c3ccc4oc5ccccc5c4c3-c3ccc4oc5ccccc5c4c3)cc2)cc1]

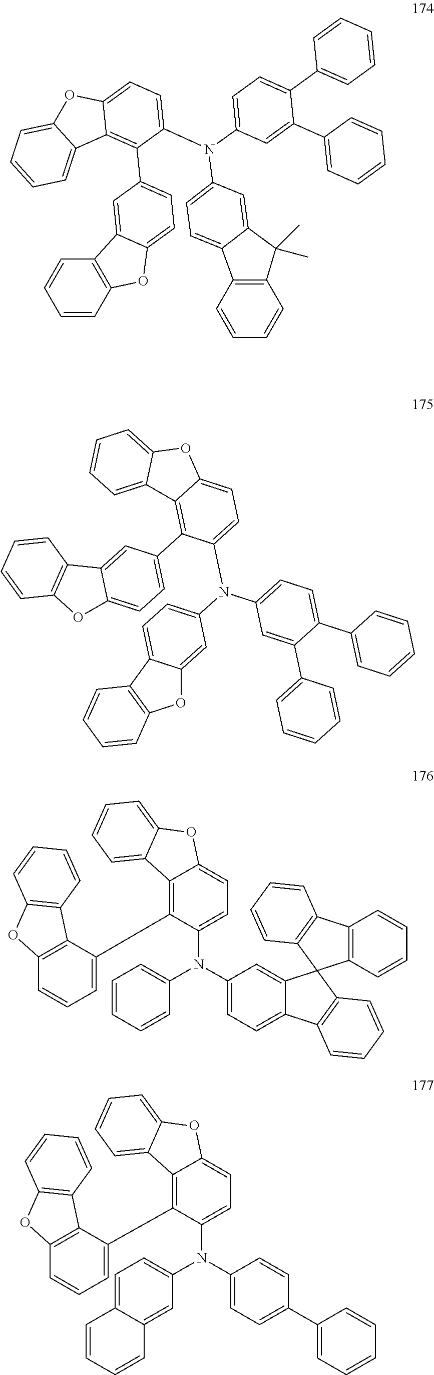 [SMILES: CC1(C)c2ccccc2-c2ccc(N(c3ccc(-c4ccccc4)c(-c4ccccc4)c3)c3ccc4oc5ccccc5c4c3-c3ccc4oc5ccccc5c4c3)cc21 ; c1ccc(-c2ccc(N(c3ccc4c(c3)oc3ccccc34)c3ccc4oc5ccccc5c4c3-c3ccc4oc5ccccc5c4c3)cc2-c2ccccc2)cc1 ; c1ccc(N(c2ccc3c(c2)C2(c4ccccc4-c4ccccc42)c2ccccc2-3)c2ccc3oc4ccccc4c3c2-c2cccc3oc4ccccc4c23)cc1 ; c1ccc(-c2ccc(N(c3ccc4ccccc4c3)c3ccc4oc5ccccc5c4c3-c3cccc4oc5ccccc5c34)cc2)cc1]

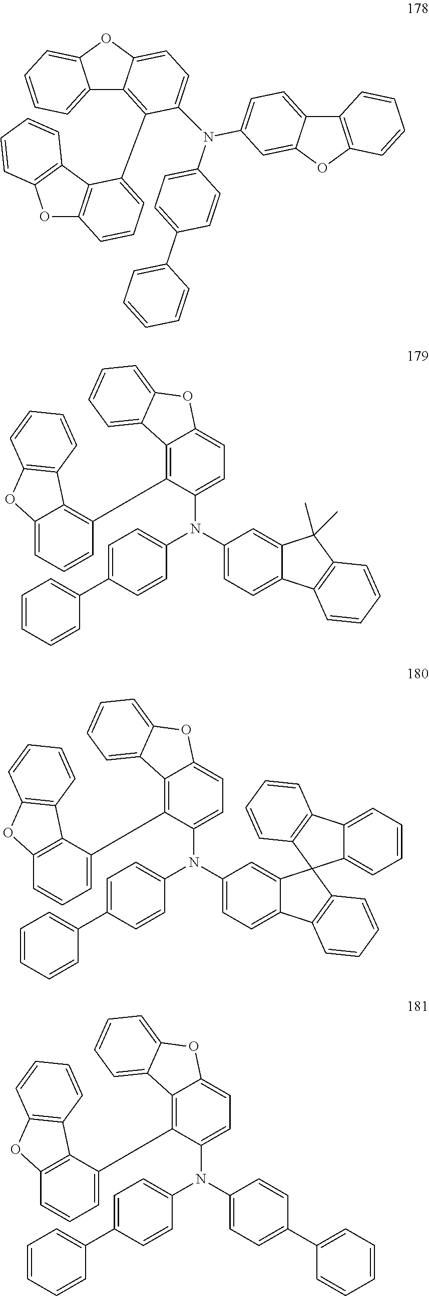

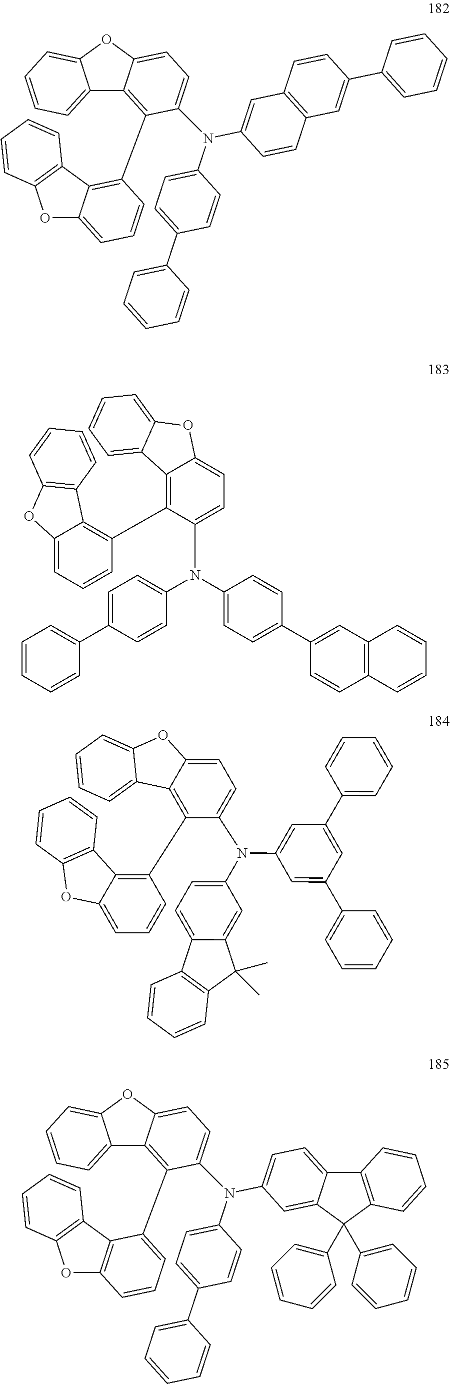

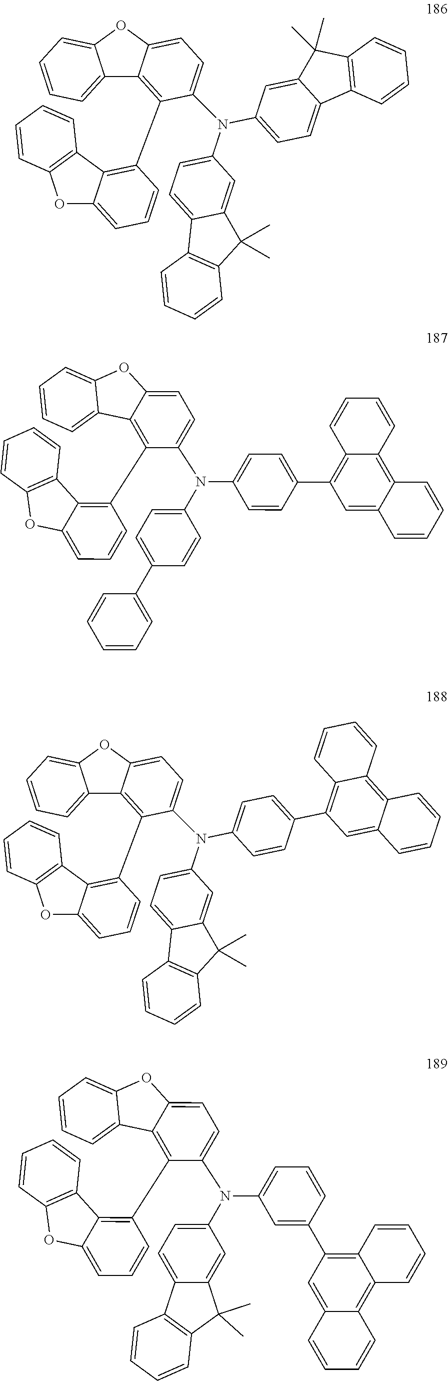 [SMILES: CC1(C)c2ccccc2-c2ccc(N(c3ccc4c(c3)C(C)(C)c3ccccc3-4)c3ccc4oc5ccccc5c4c3-c3cccc4oc5ccccc5c34)cc21 ; c1ccc(-c2ccc(N(c3ccc(-c4cc5ccccc5c5ccccc45)cc3)c3ccc4oc5ccccc5c4c3-c3cccc4oc5ccccc5c34)cc2)cc1 ; CC1(C)c2ccccc2-c2ccc(N(c3ccc(-c4cc5ccccc5c5ccccc45)cc3)c3ccc4oc5ccccc5c4c3-c3cccc4oc5ccccc5c34)cc21 ; CC1(C)c2ccccc2-c2ccc(N(c3cccc(-c4cc5ccccc5c5ccccc45)c3)c3ccc4oc5ccccc5c4c3-c3cccc4oc5ccccc5c34)cc21]

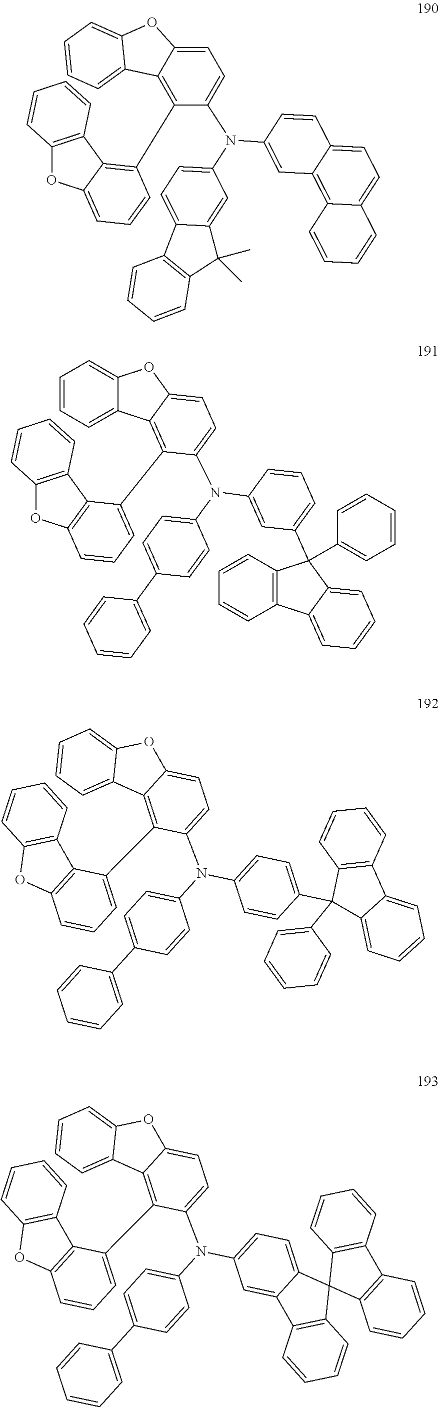

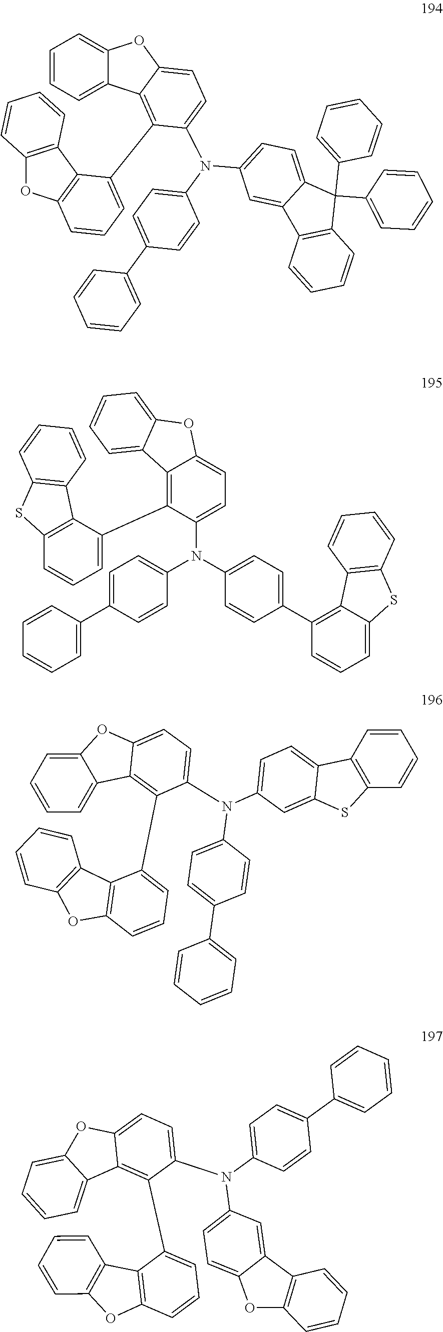 [SMILES: c1ccc(-c2ccc(N(c3ccc4c(c3)-c3ccccc3C4(c3ccccc3)c3ccccc3)c3ccc4oc5ccccc5c4c3-c3cccc4oc5ccccc5c34)cc2)cc1 ; c1ccc(-c2ccc(N(c3ccc(-c4cccc5sc6ccccc6c45)cc3)c3ccc4oc5ccccc5c4c3-c3cccc4sc5ccccc5c34)cc2)cc1 ; c1ccc(-c2ccc(N(c3ccc4c(c3)sc3ccccc34)c3ccc4oc5ccccc5c4c3-c3cccc4oc5ccccc5c34)cc2)cc1 ; c1ccc(-c2ccc(N(c3ccc4oc5ccccc5c4c3)c3ccc4oc5ccccc5c4c3-c3cccc4oc5ccccc5c34)cc2)cc1]

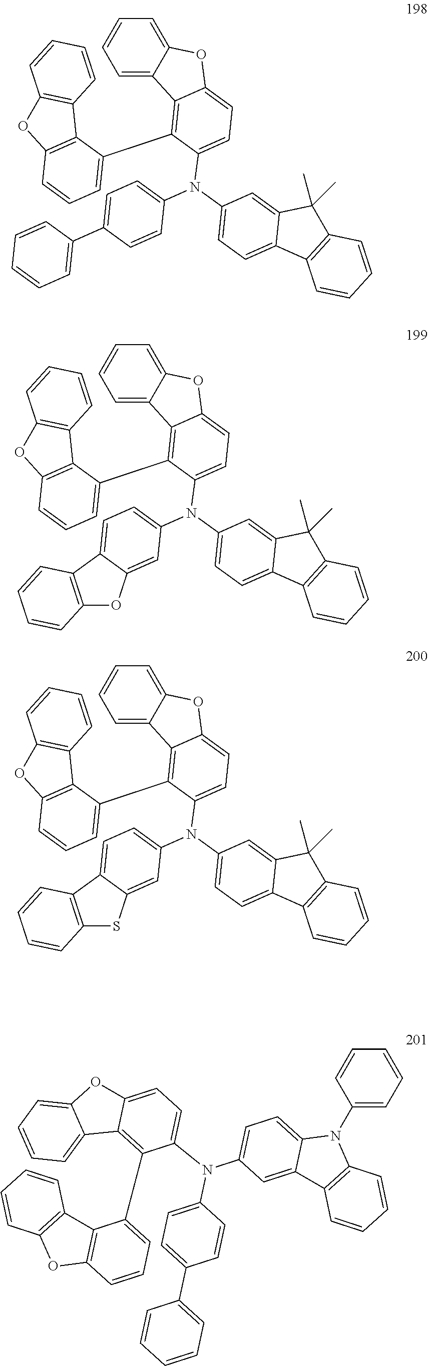 [SMILES: CC1(C)c2ccccc2-c2ccc(N(c3ccc(-c4ccccc4)cc3)c3ccc4oc5ccccc5c4c3-c3cccc4oc5ccccc5c34)cc21 ; CC1(C)c2ccccc2-c2ccc(N(c3ccc4c(c3)oc3ccccc34)c3ccc4oc5ccccc5c4c3-c3cccc4oc5ccccc5c34)cc21 ; CC1(C)c2ccccc2-c2ccc(N(c3ccc4c(c3)sc3ccccc34)c3ccc4oc5ccccc5c4c3-c3cccc4oc5ccccc5c34)cc21 ; c1ccc(-c2ccc(N(c3ccc4c(c3)c3ccccc3n4-c3ccccc3)c3ccc4oc5ccccc5c4c3-c3cccc4oc5ccccc5c34)cc2)cc1]

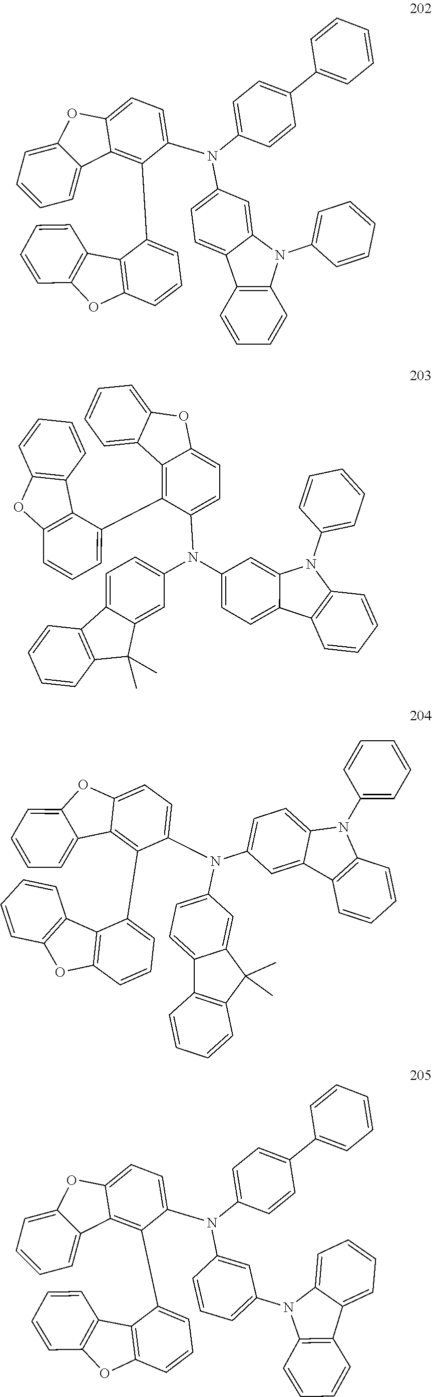 [SMILES: c1ccc(-c2ccc(N(c3ccc4c5ccccc5n(-c5ccccc5)c4c3)c3ccc4oc5ccccc5c4c3-c3cccc4oc5ccccc5c34)cc2)cc1 ; CC1(C)c2ccccc2-c2ccc(N(c3ccc4c5ccccc5n(-c5ccccc5)c4c3)c3ccc4oc5ccccc5c4c3-c3cccc4oc5ccccc5c34)cc21 ; CC1(C)c2ccccc2-c2ccc(N(c3ccc4c(c3)c3ccccc3n4-c3ccccc3)c3ccc4oc5ccccc5c4c3-c3cccc4oc5ccccc5c34)cc21 ; c1ccc(-c2ccc(N(c3cccc(-n4c5ccccc5c5ccccc54)c3)c3ccc4oc5ccccc5c4c3-c3cccc4oc5ccccc5c34)cc2)cc1]

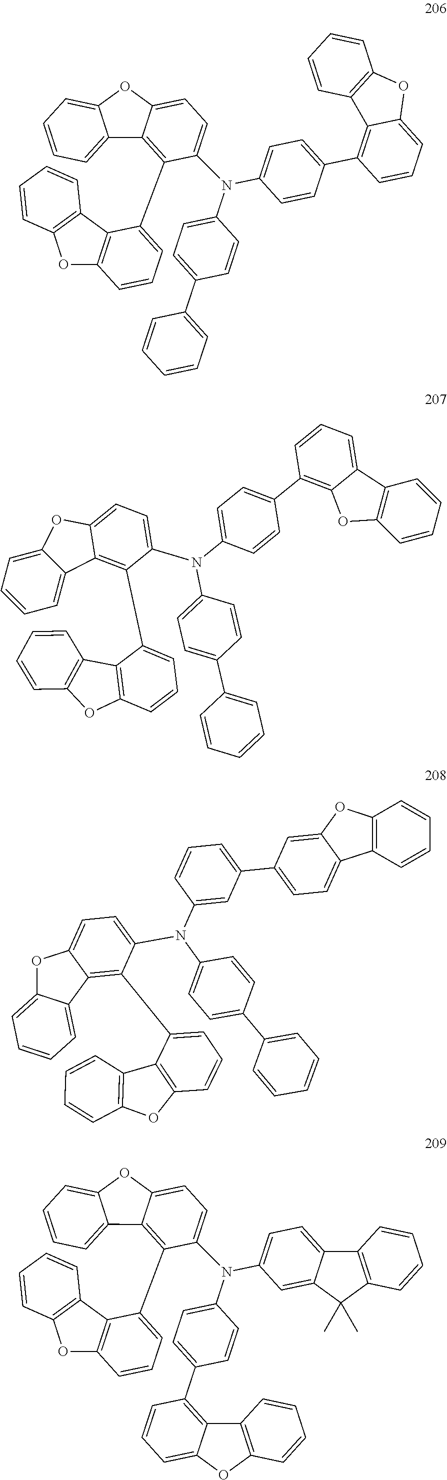 [SMILES: c1ccc(-c2ccc(N(c3ccc(-c4cccc5oc6ccccc6c45)cc3)c3ccc4oc5ccccc5c4c3-c3cccc4oc5ccccc5c34)cc2)cc1 ; c1ccc(-c2ccc(N(c3ccc(-c4cccc5c4oc4ccccc45)cc3)c3ccc4oc5ccccc5c4c3-c3cccc4oc5ccccc5c34)cc2)cc1 ; c1ccc(-c2ccc(N(c3cccc(-c4ccc5c(c4)oc4ccccc45)c3)c3ccc4oc5ccccc5c4c3-c3cccc4oc5ccccc5c34)cc2)cc1 ; CC1(C)c2ccccc2-c2ccc(N(c3ccc(-c4cccc5oc6ccccc6c45)cc3)c3ccc4oc5ccccc5c4c3-c3cccc4oc5ccccc5c34)cc21]

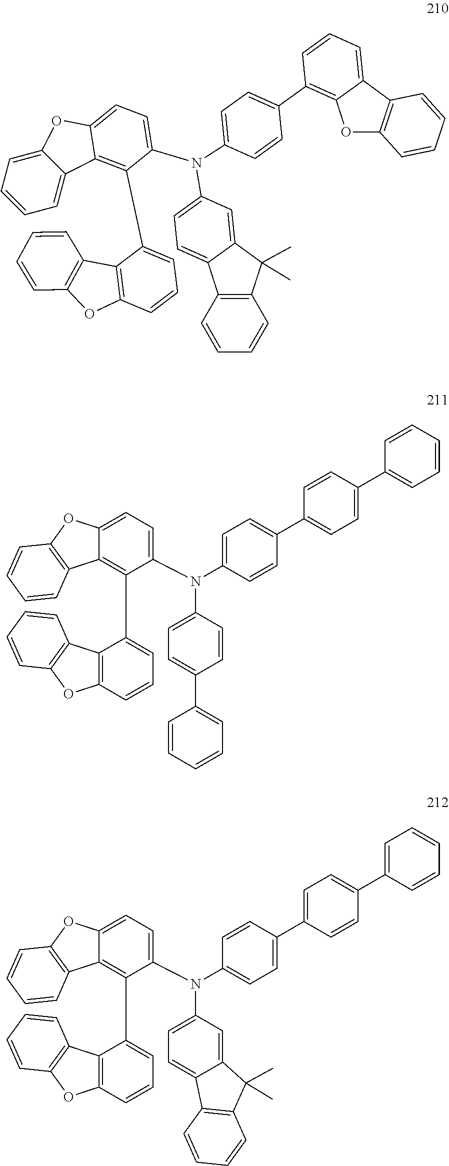 [SMILES: CC1(C)c2ccccc2-c2ccc(N(c3ccc(-c4cccc5c4oc4ccccc45)cc3)c3ccc4oc5ccccc5c4c3-c3cccc4oc5ccccc5c34)cc21 ; c1ccc(-c2ccc(-c3ccc(N(c4ccc(-c5ccccc5)cc4)c4ccc5oc6ccccc6c5c4-c4cccc5oc6ccccc6c45)cc3)cc2)cc1 ; CC1(C)c2ccccc2-c2ccc(N(c3ccc(-c4ccc(-c5ccccc5)cc4)cc3)c3ccc4oc5ccccc5c4c3-c3cccc4oc5ccccc5c34)cc21]

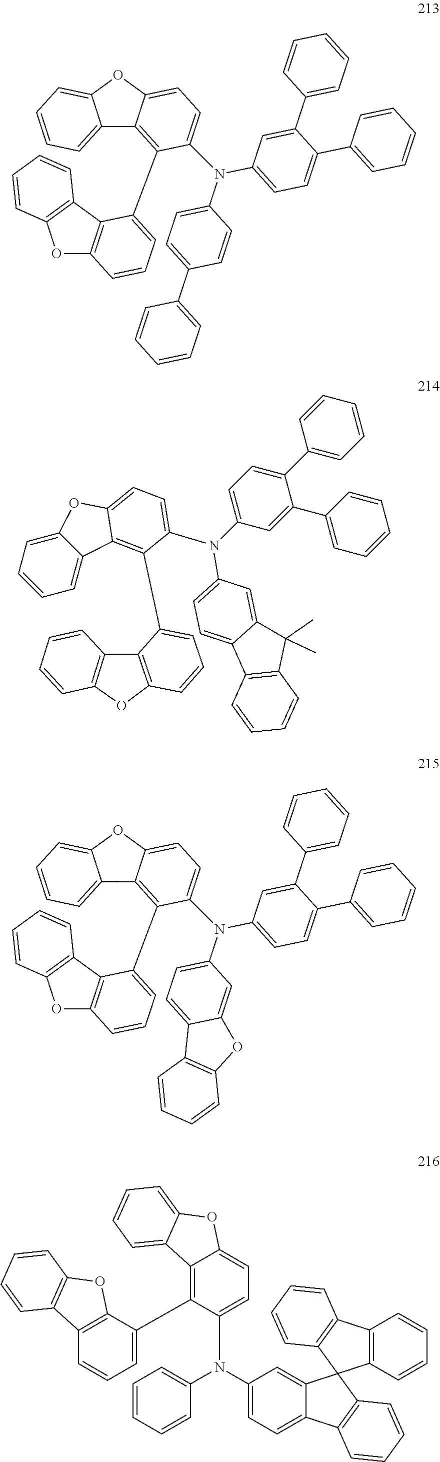 [SMILES: c1ccc(-c2ccc(N(c3ccc(-c4ccccc4)c(-c4ccccc4)c3)c3ccc4oc5ccccc5c4c3-c3cccc4oc5ccccc5c34)cc2)cc1 ; CC1(C)c2ccccc2-c2ccc(N(c3ccc(-c4ccccc4)c(-c4ccccc4)c3)c3ccc4oc5ccccc5c4c3-c3cccc4oc5ccccc5c34)cc21 ; c1ccc(-c2ccc(N(c3ccc4c(c3)oc3ccccc34)c3ccc4oc5ccccc5c4c3-c3cccc4oc5ccccc5c34)cc2-c2ccccc2)cc1 ; c1ccc(N(c2ccc3c(c2)C2(c4ccccc4-c4ccccc42)c2ccccc2-3)c2ccc3oc4ccccc4c3c2-c2cccc3c2oc2ccccc23)cc1]

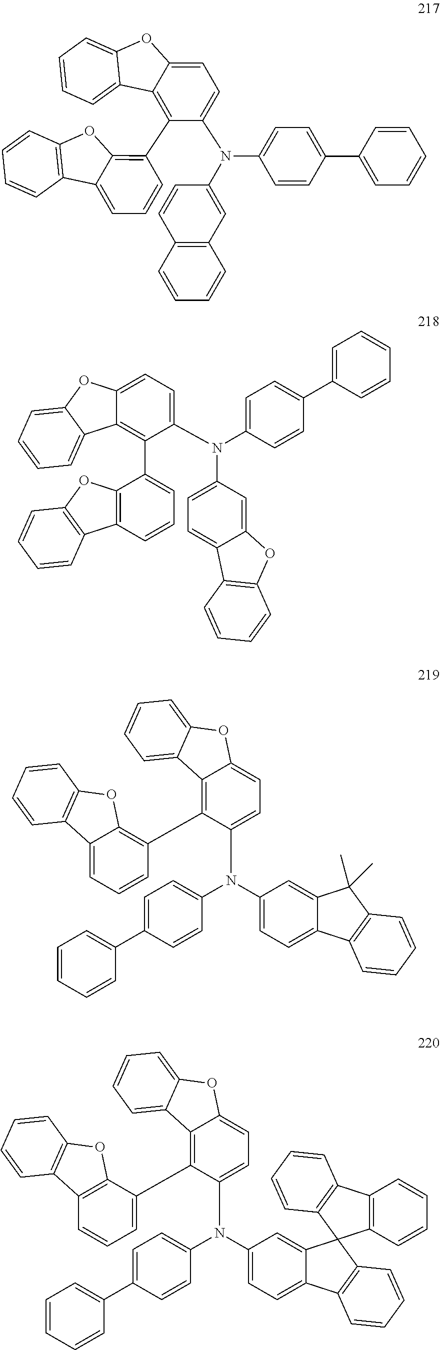 [SMILES: c1ccc(-c2ccc(N(c3ccc4ccccc4c3)c3ccc4oc5ccccc5c4c3-c3cccc4c3oc3ccccc34)cc2)cc1 ; c1ccc(-c2ccc(N(c3ccc4c(c3)oc3ccccc34)c3ccc4oc5ccccc5c4c3-c3cccc4c3oc3ccccc34)cc2)cc1 ; CC1(C)c2ccccc2-c2ccc(N(c3ccc(-c4ccccc4)cc3)c3ccc4oc5ccccc5c4c3-c3cccc4c3oc3ccccc34)cc21 ; c1ccc(-c2ccc(N(c3ccc4c(c3)C3(c5ccccc5-c5ccccc53)c3ccccc3-4)c3ccc4oc5ccccc5c4c3-c3cccc4c3oc3ccccc34)cc2)cc1]

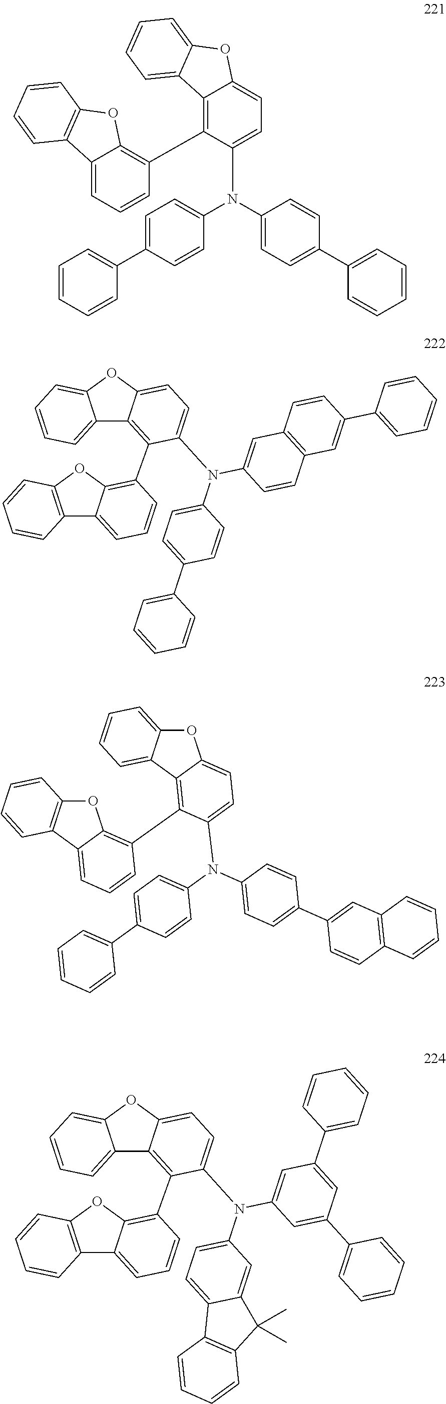 [SMILES: c1ccc(-c2ccc(N(c3ccc(-c4ccccc4)cc3)c3ccc4oc5ccccc5c4c3-c3cccc4c3oc3ccccc34)cc2)cc1 ; c1ccc(-c2ccc(N(c3ccc4cc(-c5ccccc5)ccc4c3)c3ccc4oc5ccccc5c4c3-c3cccc4c3oc3ccccc34)cc2)cc1 ; c1ccc(-c2ccc(N(c3ccc(-c4ccc5ccccc5c4)cc3)c3ccc4oc5ccccc5c4c3-c3cccc4c3oc3ccccc34)cc2)cc1 ; CC1(C)c2ccccc2-c2ccc(N(c3cc(-c4ccccc4)cc(-c4ccccc4)c3)c3ccc4oc5ccccc5c4c3-c3cccc4c3oc3ccccc34)cc21]

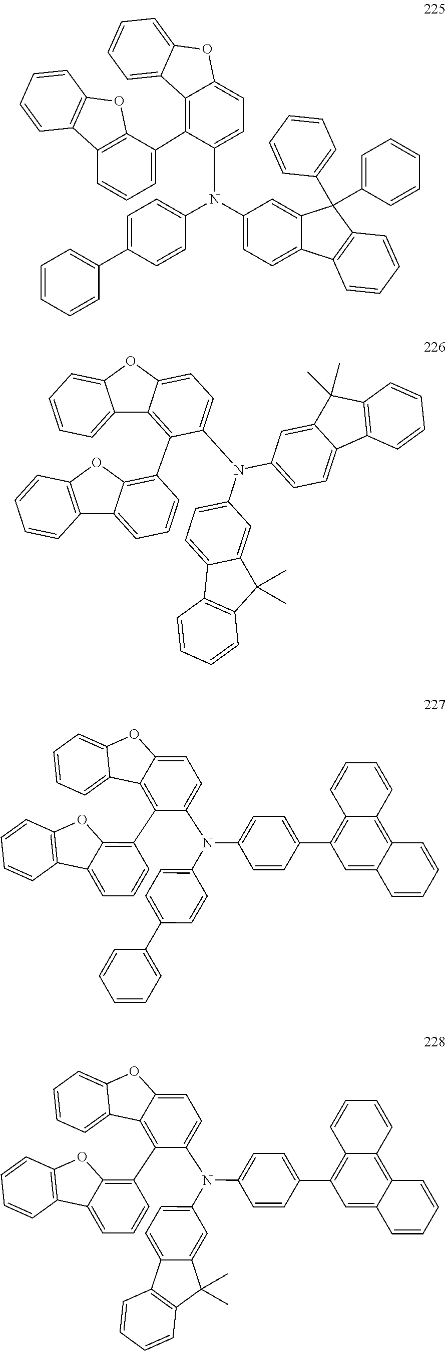 [SMILES: c1ccc(-c2ccc(N(c3ccc4c(c3)C(c3ccccc3)(c3ccccc3)c3ccccc3-4)c3ccc4oc5ccccc5c4c3-c3cccc4c3oc3ccccc34)cc2)cc1 ; CC1(C)c2ccccc2-c2ccc(N(c3ccc4c(c3)C(C)(C)c3ccccc3-4)c3ccc4oc5ccccc5c4c3-c3cccc4c3oc3ccccc34)cc21 ; c1ccc(-c2ccc(N(c3ccc(-c4cc5ccccc5c5ccccc45)cc3)c3ccc4oc5ccccc5c4c3-c3cccc4c3oc3ccccc34)cc2)cc1 ; CC1(C)c2ccccc2-c2ccc(N(c3ccc(-c4cc5ccccc5c5ccccc45)cc3)c3ccc4oc5ccccc5c4c3-c3cccc4c3oc3ccccc34)cc21]

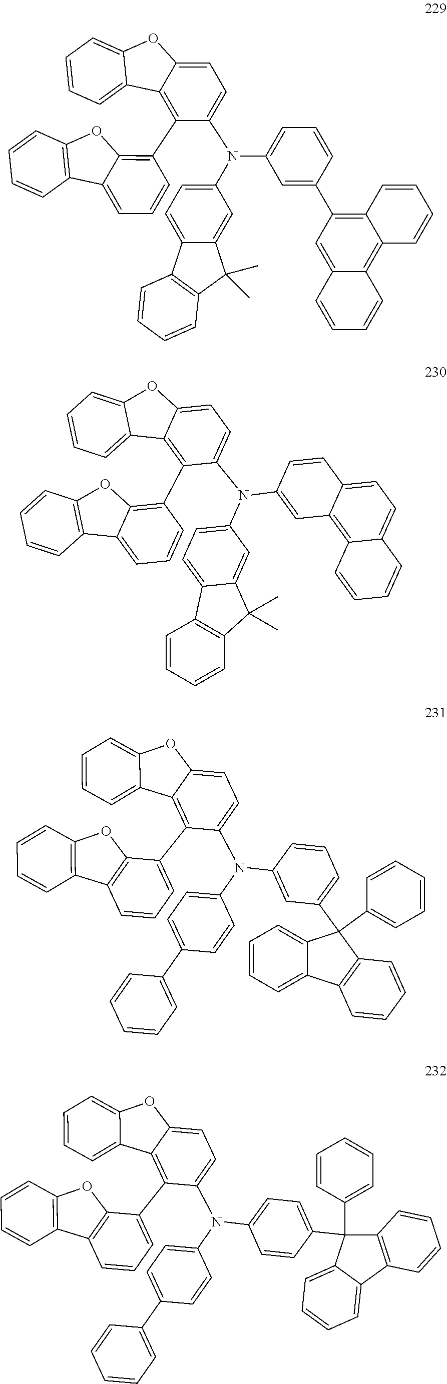 [SMILES: CC1(C)c2ccccc2-c2ccc(N(c3cccc(-c4cc5ccccc5c5ccccc45)c3)c3ccc4oc5ccccc5c4c3-c3cccc4c3oc3ccccc34)cc21 ; CC1(C)c2ccccc2-c2ccc(N(c3ccc4ccc5ccccc5c4c3)c3ccc4oc5ccccc5c4c3-c3cccc4c3oc3ccccc34)cc21 ; c1ccc(-c2ccc(N(c3cccc(C4(c5ccccc5)c5ccccc5-c5ccccc54)c3)c3ccc4oc5ccccc5c4c3-c3cccc4c3oc3ccccc34)cc2)cc1 ; c1ccc(-c2ccc(N(c3ccc(C4(c5ccccc5)c5ccccc5-c5ccccc54)cc3)c3ccc4oc5ccccc5c4c3-c3cccc4c3oc3ccccc34)cc2)cc1]

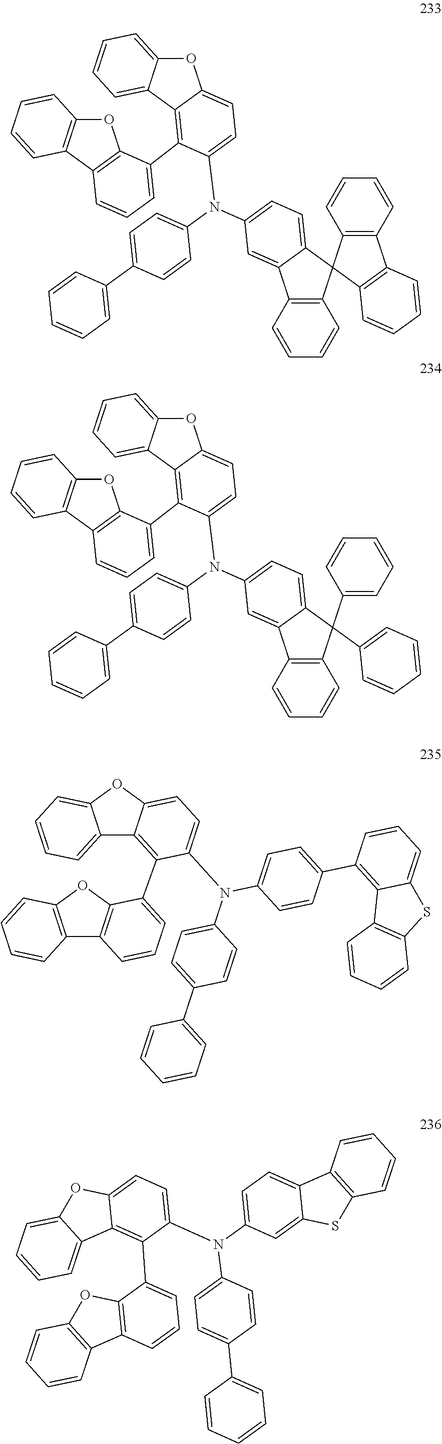 [SMILES: c1ccc(-c2ccc(N(c3ccc4c(c3)-c3ccccc3C43c4ccccc4-c4ccccc43)c3ccc4oc5ccccc5c4c3-c3cccc4c3oc3ccccc34)cc2)cc1 ; c1ccc(-c2ccc(N(c3ccc4c(c3)-c3ccccc3C4(c3ccccc3)c3ccccc3)c3ccc4oc5ccccc5c4c3-c3cccc4c3oc3ccccc34)cc2)cc1 ; c1ccc(-c2ccc(N(c3ccc(-c4cccc5sc6ccccc6c45)cc3)c3ccc4oc5ccccc5c4c3-c3cccc4c3oc3ccccc34)cc2)cc1 ; c1ccc(-c2ccc(N(c3ccc4c(c3)sc3ccccc34)c3ccc4oc5ccccc5c4c3-c3cccc4c3oc3ccccc34)cc2)cc1]

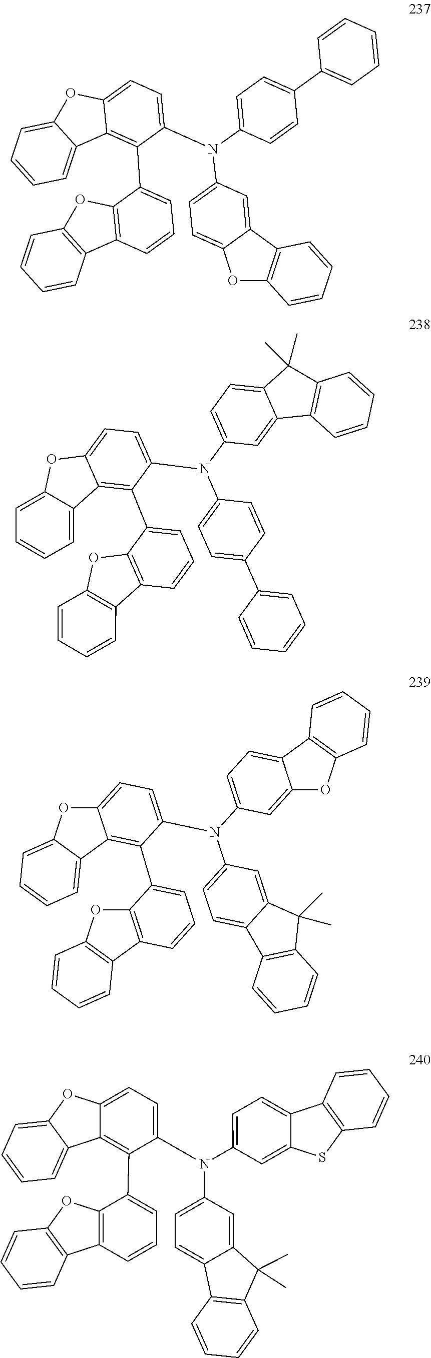

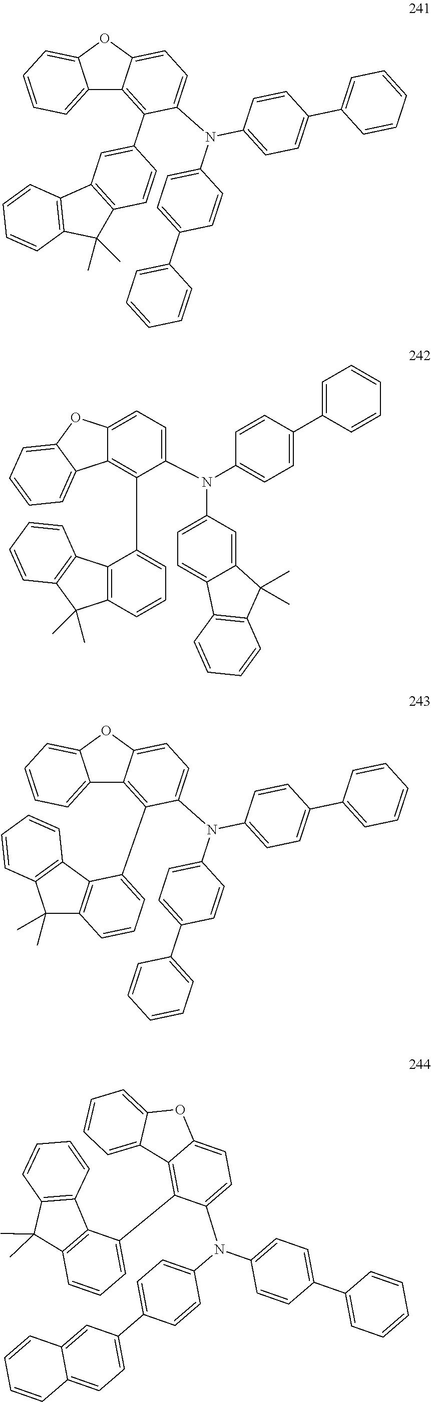 [SMILES: CC1(C)c2ccccc2-c2cc(-c3c(N(c4ccc(-c5ccccc5)cc4)c4ccc(-c5ccccc5)cc4)ccc4oc5ccccc5c34)ccc21 ; CC1(C)c2ccccc2-c2ccc(N(c3ccc(-c4ccccc4)cc3)c3ccc4oc5ccccc5c4c3-c3cccc4c3-c3ccccc3C4(C)C)cc21 ; CC1(C)c2ccccc2-c2c(-c3c(N(c4ccc(-c5ccccc5)cc4)c4ccc(-c5ccccc5)cc4)ccc4oc5ccccc5c34)cccc21 ; CC1(C)c2ccccc2-c2c(-c3c(N(c4ccc(-c5ccccc5)cc4)c4ccc(-c5ccc6ccccc6c5)cc4)ccc4oc5ccccc5c34)cccc21]

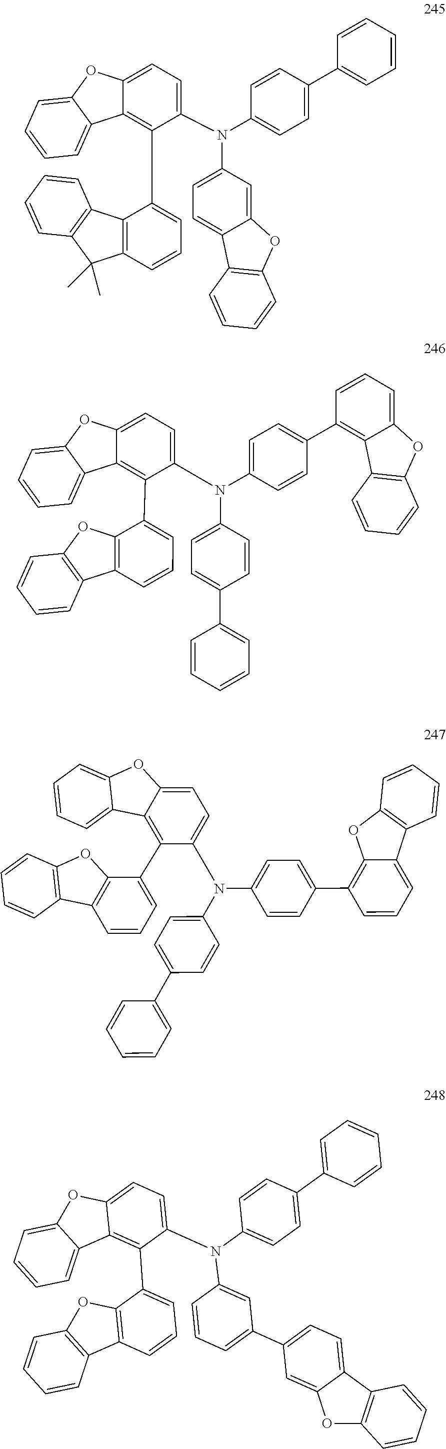 [SMILES: CC1(C)c2ccccc2-c2c(-c3c(N(c4ccc(-c5ccccc5)cc4)c4ccc5c(c4)oc4ccccc45)ccc4oc5ccccc5c34)cccc21 ; c1ccc(-c2ccc(N(c3ccc(-c4cccc5oc6ccccc6c45)cc3)c3ccc4oc5ccccc5c4c3-c3cccc4c3oc3ccccc34)cc2)cc1 ; c1ccc(-c2ccc(N(c3ccc(-c4cccc5c4oc4ccccc45)cc3)c3ccc4oc5ccccc5c4c3-c3cccc4c3oc3ccccc34)cc2)cc1 ; c1ccc(-c2ccc(N(c3cccc(-c4ccc5c(c4)oc4ccccc45)c3)c3ccc4oc5ccccc5c4c3-c3cccc4c3oc3ccccc34)cc2)cc1]

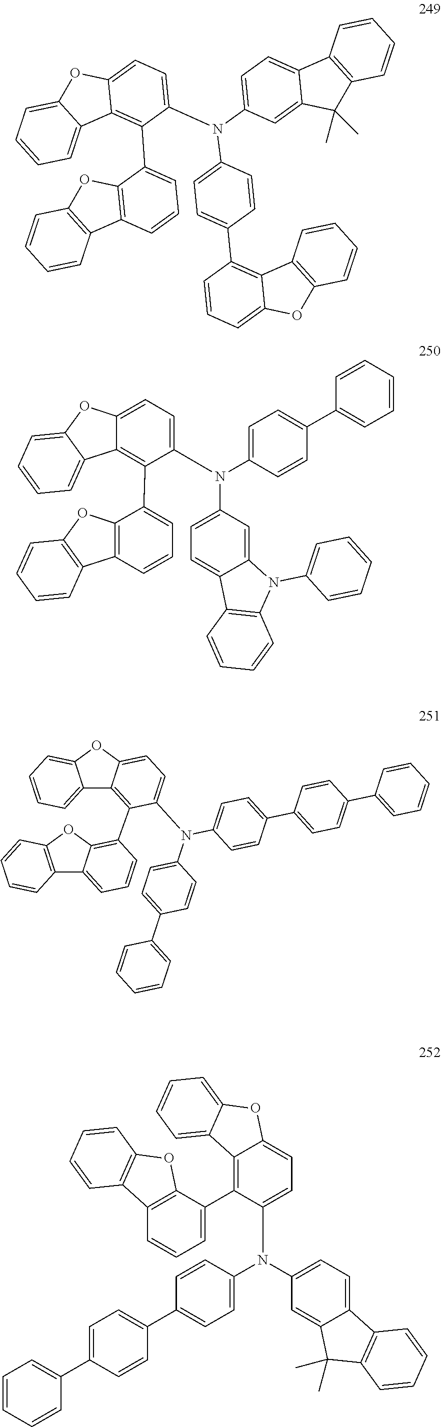 [SMILES: CC1(C)c2ccccc2-c2ccc(N(c3ccc(-c4cccc5oc6ccccc6c45)cc3)c3ccc4oc5ccccc5c4c3-c3cccc4c3oc3ccccc34)cc21 ; c1ccc(-c2ccc(N(c3ccc4c5ccccc5n(-c5ccccc5)c4c3)c3ccc4oc5ccccc5c4c3-c3cccc4c3oc3ccccc34)cc2)cc1 ; c1ccc(-c2ccc(-c3ccc(N(c4ccc(-c5ccccc5)cc4)c4ccc5oc6ccccc6c5c4-c4cccc5c4oc4ccccc45)cc3)cc2)cc1 ; CC1(C)c2ccccc2-c2ccc(N(c3ccc(-c4ccc(-c5ccccc5)cc4)cc3)c3ccc4oc5ccccc5c4c3-c3cccc4c3oc3ccccc34)cc21]

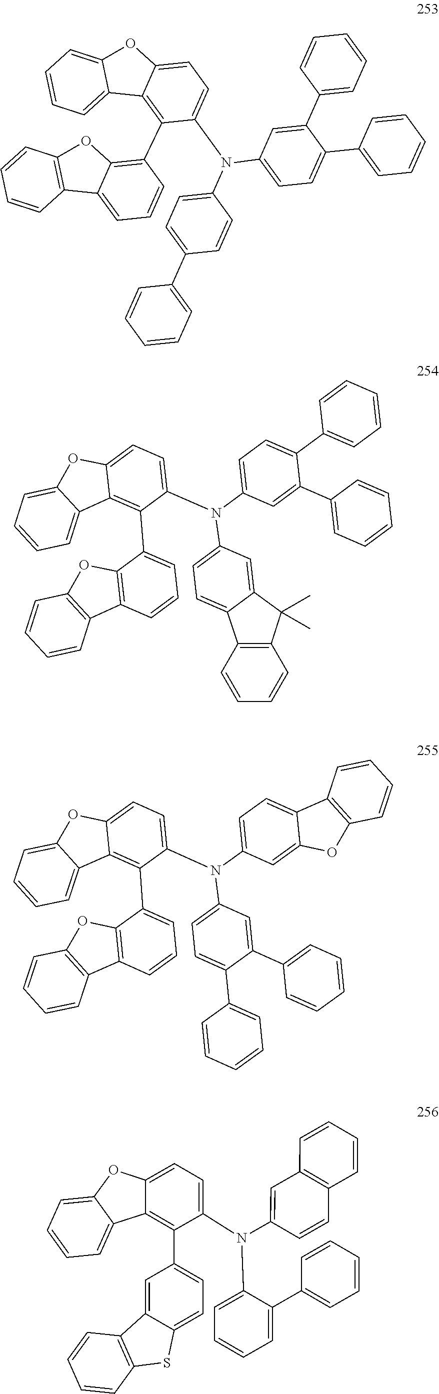 [SMILES: c1ccc(-c2ccc(N(c3ccc(-c4ccccc4)c(-c4ccccc4)c3)c3ccc4oc5ccccc5c4c3-c3cccc4c3oc3ccccc34)cc2)cc1 ; CC1(C)c2ccccc2-c2ccc(N(c3ccc(-c4ccccc4)c(-c4ccccc4)c3)c3ccc4oc5ccccc5c4c3-c3cccc4c3oc3ccccc34)cc21 ; c1ccc(-c2ccc(N(c3ccc4c(c3)oc3ccccc34)c3ccc4oc5ccccc5c4c3-c3cccc4c3oc3ccccc34)cc2-c2ccccc2)cc1 ; c1ccc(-c2ccccc2N(c2ccc3ccccc3c2)c2ccc3oc4ccccc4c3c2-c2ccc3sc4ccccc4c3c2)cc1]

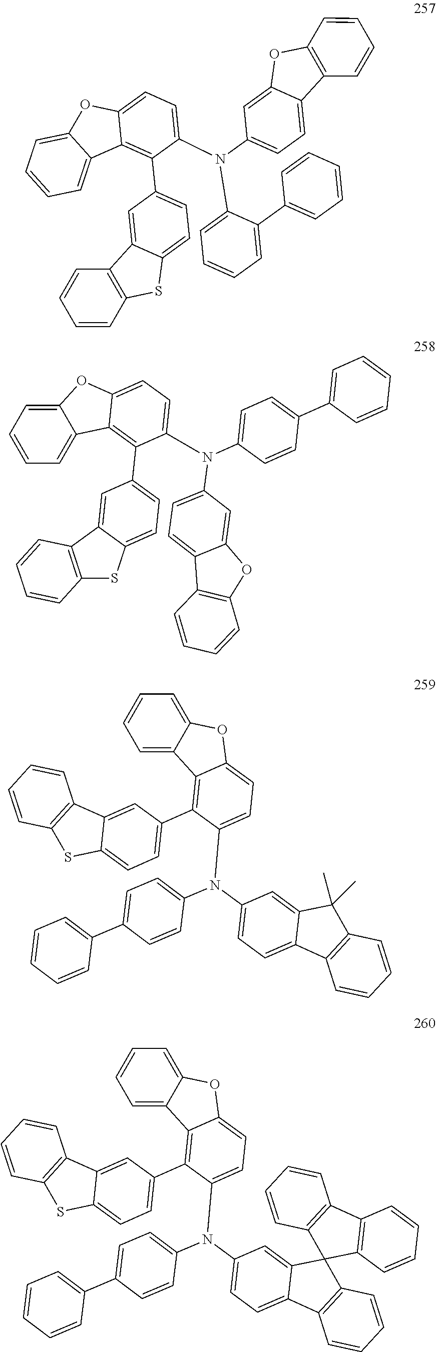 [SMILES: c1ccc(-c2ccccc2N(c2ccc3c(c2)oc2ccccc23)c2ccc3oc4ccccc4c3c2-c2ccc3sc4ccccc4c3c2)cc1 ; c1ccc(-c2ccc(N(c3ccc4c(c3)oc3ccccc34)c3ccc4oc5ccccc5c4c3-c3ccc4sc5ccccc5c4c3)cc2)cc1 ; CC1(C)c2ccccc2-c2ccc(N(c3ccc(-c4ccccc4)cc3)c3ccc4oc5ccccc5c4c3-c3ccc4sc5ccccc5c4c3)cc21 ; c1ccc(-c2ccc(N(c3ccc4c(c3)C3(c5ccccc5-c5ccccc53)c3ccccc3-4)c3ccc4oc5ccccc5c4c3-c3ccc4sc5ccccc5c4c3)cc2)cc1]

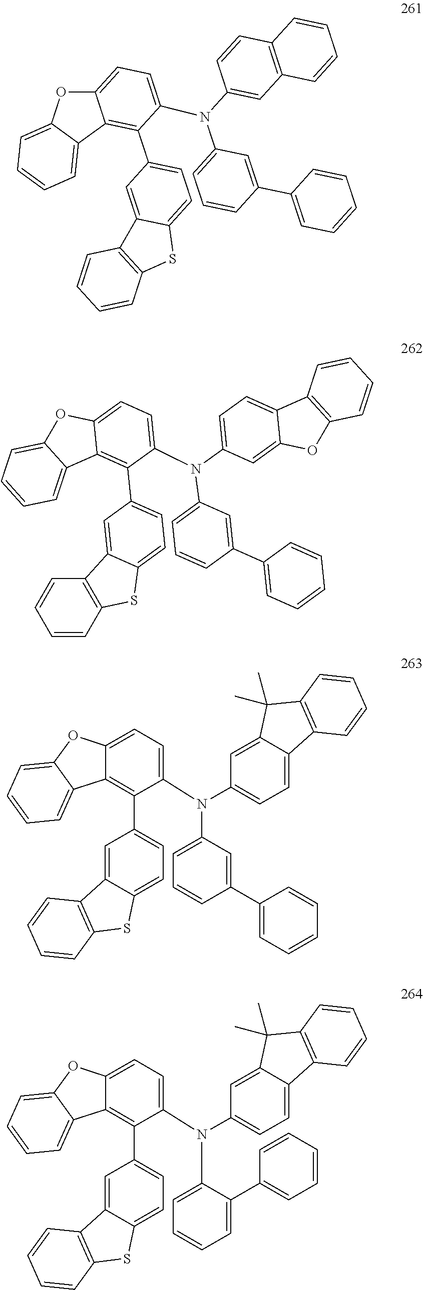 [SMILES: c1ccc(-c2cccc(N(c3ccc4ccccc4c3)c3ccc4oc5ccccc5c4c3-c3ccc4sc5ccccc5c4c3)c2)cc1 ; c1ccc(-c2cccc(N(c3ccc4c(c3)oc3ccccc34)c3ccc4oc5ccccc5c4c3-c3ccc4sc5ccccc5c4c3)c2)cc1 ; CC1(C)c2ccccc2-c2ccc(N(c3cccc(-c4ccccc4)c3)c3ccc4oc5ccccc5c4c3-c3ccc4sc5ccccc5c4c3)cc21 ; CC1(C)c2ccccc2-c2ccc(N(c3ccccc3-c3ccccc3)c3ccc4oc5ccccc5c4c3-c3ccc4sc5ccccc5c4c3)cc21]

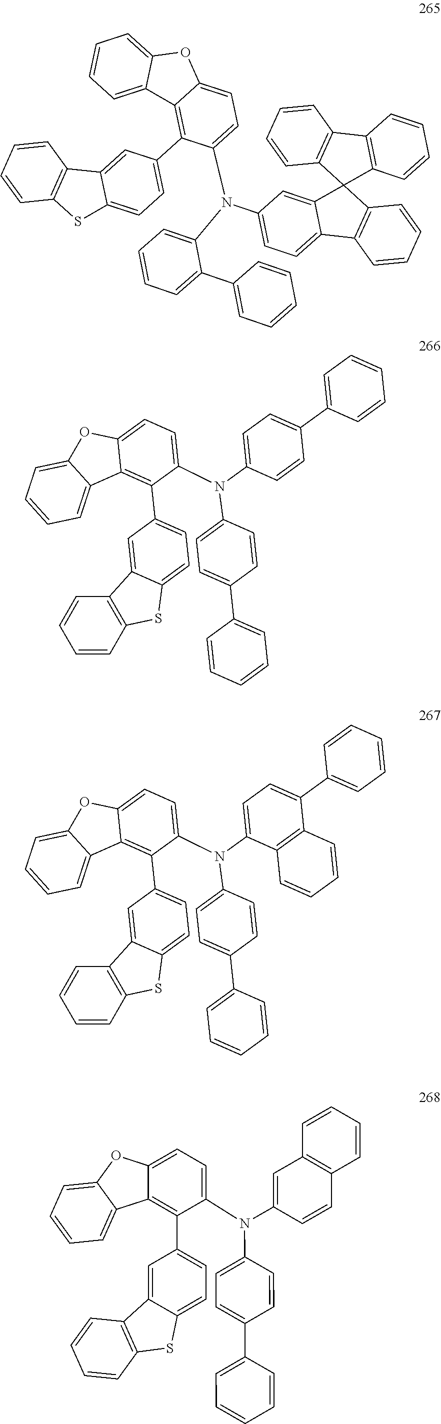 [SMILES: c1ccc(-c2ccccc2N(c2ccc3c(c2)C2(c4ccccc4-c4ccccc42)c2ccccc2-3)c2ccc3oc4ccccc4c3c2-c2ccc3sc4ccccc4c3c2)cc1 ; c1ccc(-c2ccc(N(c3ccc(-c4ccccc4)cc3)c3ccc4oc5ccccc5c4c3-c3ccc4sc5ccccc5c4c3)cc2)cc1 ; c1ccc(-c2ccc(N(c3ccc4oc5ccccc5c4c3-c3ccc4sc5ccccc5c4c3)c3ccc(-c4ccccc4)c4ccccc34)cc2)cc1 ; c1ccc(-c2ccc(N(c3ccc4ccccc4c3)c3ccc4oc5ccccc5c4c3-c3ccc4sc5ccccc5c4c3)cc2)cc1]

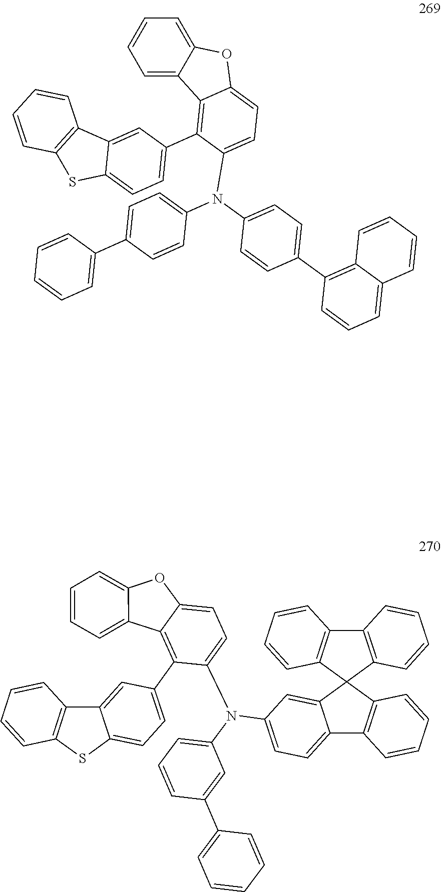 [SMILES: c1ccc(-c2ccc(N(c3ccc(-c4cccc5ccccc45)cc3)c3ccc4oc5ccccc5c4c3-c3ccc4sc5ccccc5c4c3)cc2)cc1 ; c1ccc(-c2cccc(N(c3ccc4c(c3)C3(c5ccccc5-c5ccccc53)c3ccccc3-4)c3ccc4oc5ccccc5c4c3-c3ccc4sc5ccccc5c4c3)c2)cc1]

[chemical structure]

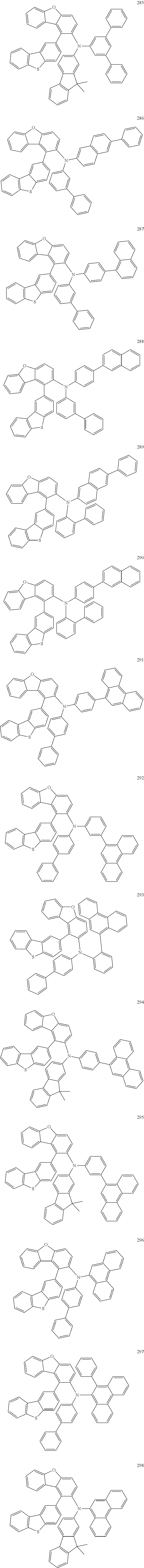

[chemical structure]

[chemical structure]

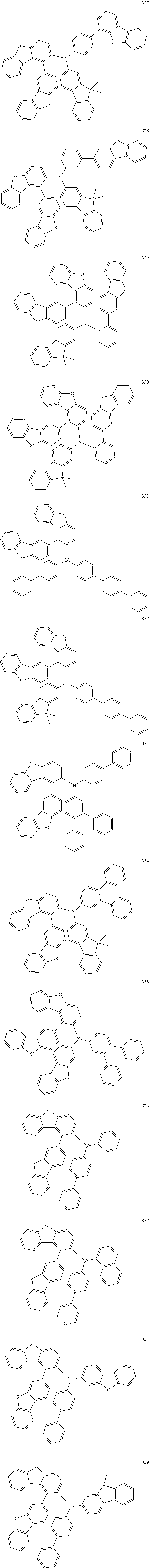

[chemical structure]

[chemical structure]

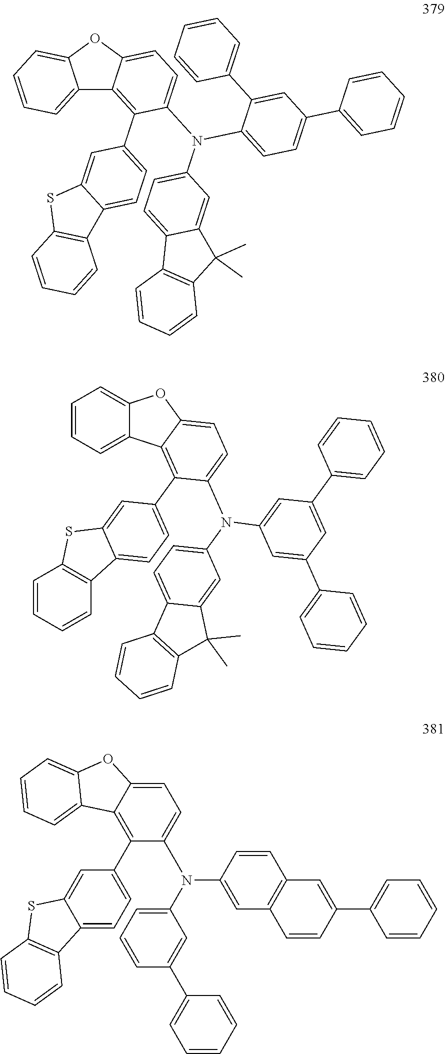 [SMILES: CC1(C)c2ccccc2-c2ccc(N(c3ccc(-c4ccccc4)cc3-c3ccccc3)c3ccc4oc5ccccc5c4c3-c3ccc4c(c3)sc3ccccc34)cc21 ; CC1(C)c2ccccc2-c2ccc(N(c3cc(-c4ccccc4)cc(-c4ccccc4)c3)c3ccc4oc5ccccc5c4c3-c3ccc4c(c3)sc3ccccc34)cc21 ; c1ccc(-c2cccc(N(c3ccc4cc(-c5ccccc5)ccc4c3)c3ccc4oc5ccccc5c4c3-c3ccc4c(c3)sc3ccccc34)c2)cc1]

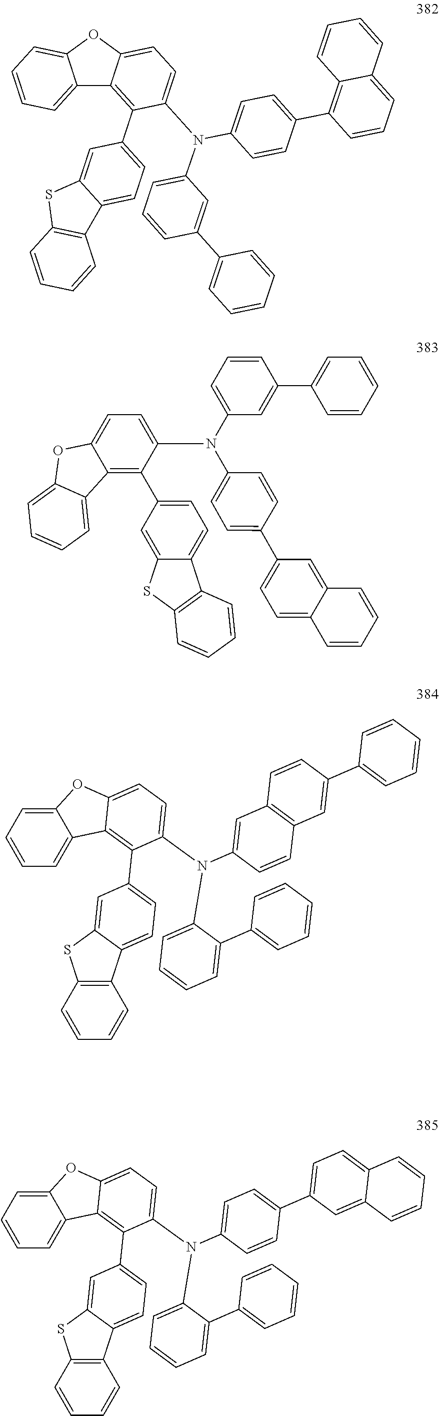

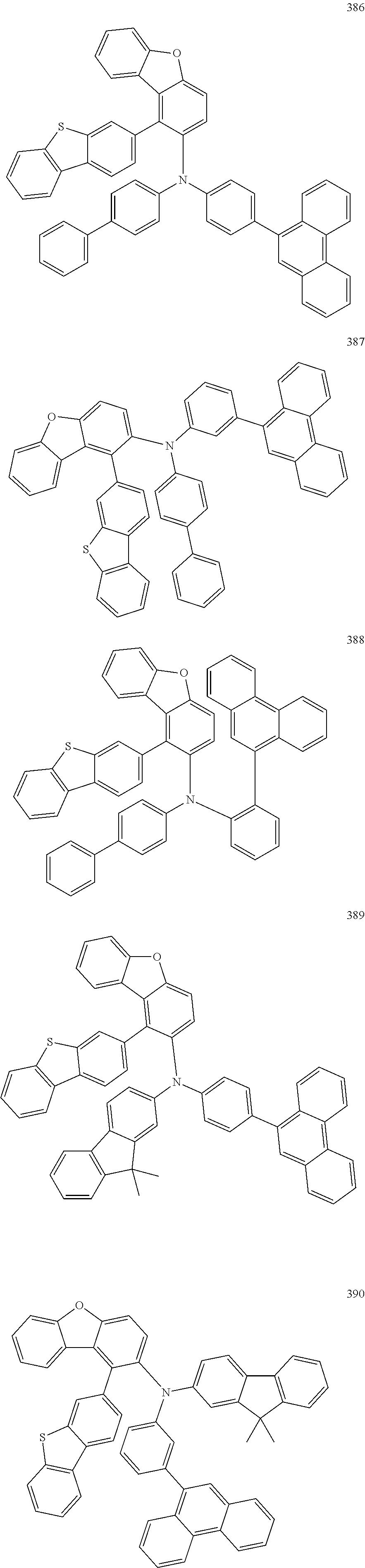

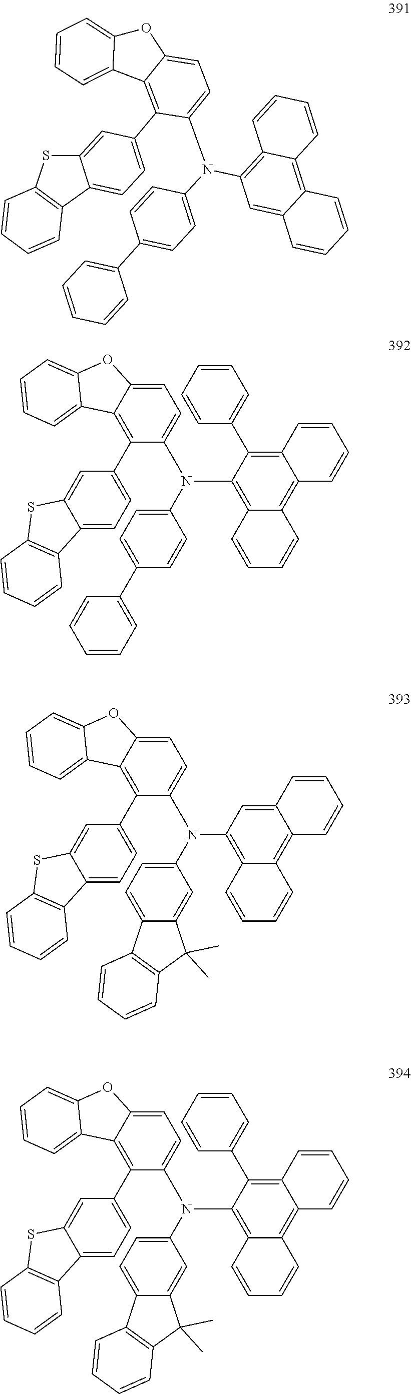

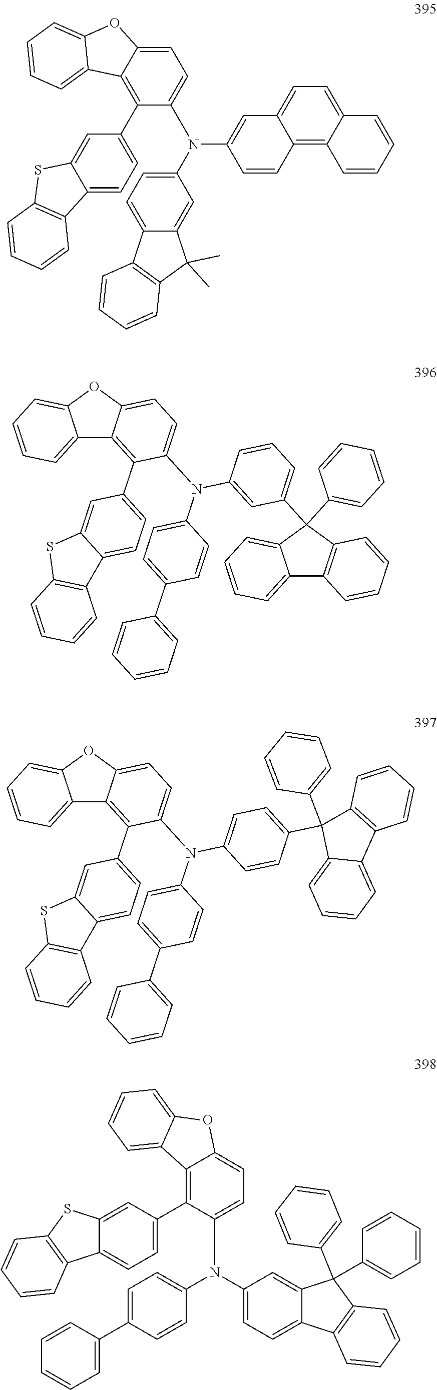 [SMILES: CC1(C)c2ccccc2-c2ccc(N(c3ccc4c(ccc5ccccc54)c3)c3ccc4oc5ccccc5c4c3-c3ccc4c(c3)sc3ccccc34)cc21 ; c1ccc(-c2ccc(N(c3cccc(C4(c5ccccc5)c5ccccc5-c5ccccc54)c3)c3ccc4oc5ccccc5c4c3-c3ccc4c(c3)sc3ccccc34)cc2)cc1 ; c1ccc(-c2ccc(N(c3ccc(C4(c5ccccc5)c5ccccc5-c5ccccc54)cc3)c3ccc4oc5ccccc5c4c3-c3ccc4c(c3)sc3ccccc34)cc2)cc1 ; c1ccc(-c2ccc(N(c3ccc4c(c3)C(c3ccccc3)(c3ccccc3)c3ccccc3-4)c3ccc4oc5ccccc5c4c3-c3ccc4c(c3)sc3ccccc34)cc2)cc1]

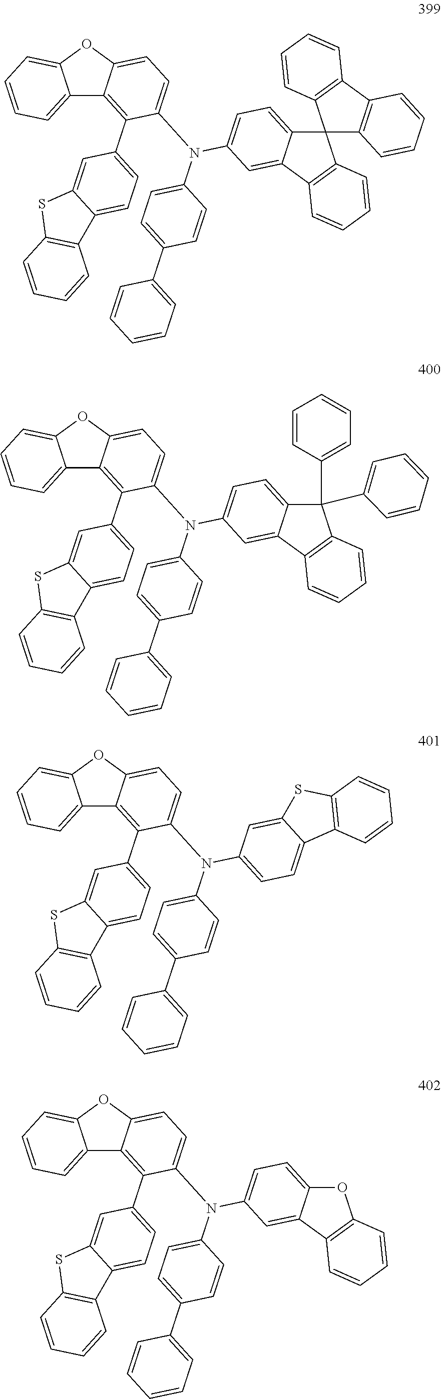 [SMILES: c1ccc(-c2ccc(N(c3ccc4c(c3)-c3ccccc3C43c4ccccc4-c4ccccc43)c3ccc4oc5ccccc5c4c3-c3ccc4c(c3)sc3ccccc34)cc2)cc1 ; c1ccc(-c2ccc(N(c3ccc4c(c3)-c3ccccc3C4(c3ccccc3)c3ccccc3)c3ccc4oc5ccccc5c4c3-c3ccc4c(c3)sc3ccccc34)cc2)cc1 ; c1ccc(-c2ccc(N(c3ccc4c(c3)sc3ccccc34)c3ccc4oc5ccccc5c4c3-c3ccc4c(c3)sc3ccccc34)cc2)cc1 ; c1ccc(-c2ccc(N(c3ccc4oc5ccccc5c4c3)c3ccc4oc5ccccc5c4c3-c3ccc4c(c3)sc3ccccc34)cc2)cc1]

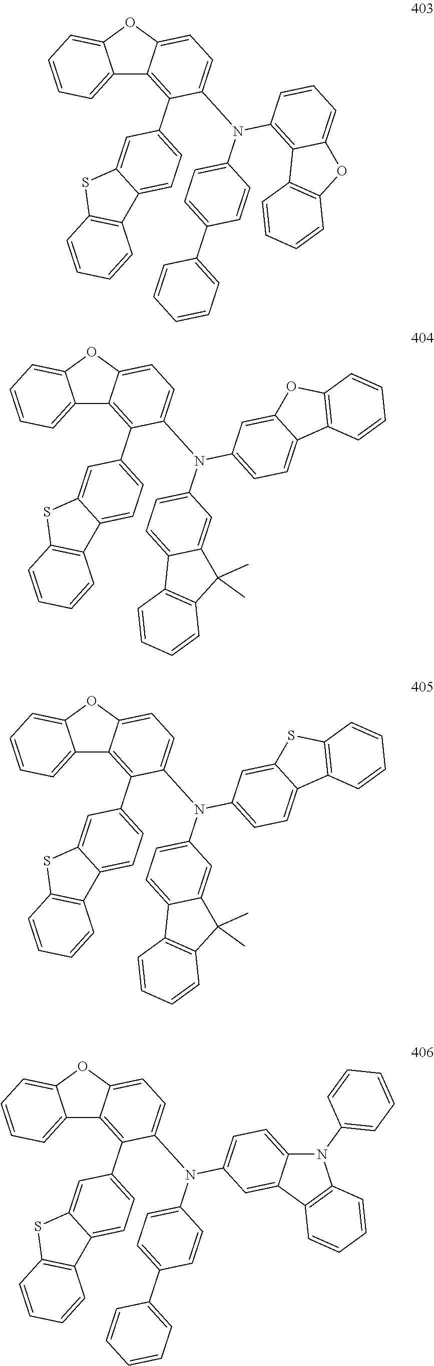 [SMILES: c1ccc(-c2ccc(N(c3ccc4oc5ccccc5c4c3-c3ccc4c(c3)sc3ccccc34)c3cccc4oc5ccccc5c34)cc2)cc1 ; CC1(C)c2ccccc2-c2ccc(N(c3ccc4c(c3)oc3ccccc34)c3ccc4oc5ccccc5c4c3-c3ccc4c(c3)sc3ccccc34)cc21 ; CC1(C)c2ccccc2-c2ccc(N(c3ccc4c(c3)sc3ccccc34)c3ccc4oc5ccccc5c4c3-c3ccc4c(c3)sc3ccccc34)cc21 ; c1ccc(-c2ccc(N(c3ccc4c(c3)c3ccccc3n4-c3ccccc3)c3ccc4oc5ccccc5c4c3-c3ccc4c(c3)sc3ccccc34)cc2)cc1]

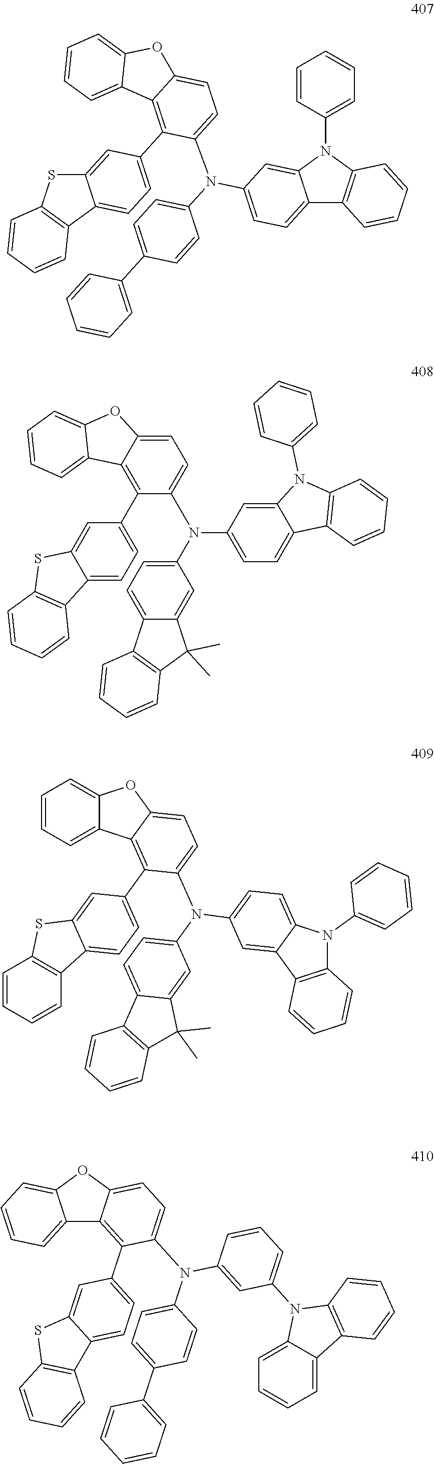 [SMILES: c1ccc(-c2ccc(N(c3ccc4c5ccccc5n(-c5ccccc5)c4c3)c3ccc4oc5ccccc5c4c3-c3ccc4c(c3)sc3ccccc34)cc2)cc1 ; CC1(C)c2ccccc2-c2ccc(N(c3ccc4c5ccccc5n(-c5ccccc5)c4c3)c3ccc4oc5ccccc5c4c3-c3ccc4c(c3)sc3ccccc34)cc21 ; CC1(C)c2ccccc2-c2ccc(N(c3ccc4c(c3)c3ccccc3n4-c3ccccc3)c3ccc4oc5ccccc5c4c3-c3ccc4c(c3)sc3ccccc34)cc21 ; c1ccc(-c2ccc(N(c3cccc(-n4c5ccccc5c5ccccc54)c3)c3ccc4oc5ccccc5c4c3-c3ccc4c(c3)sc3ccccc34)cc2)cc1]

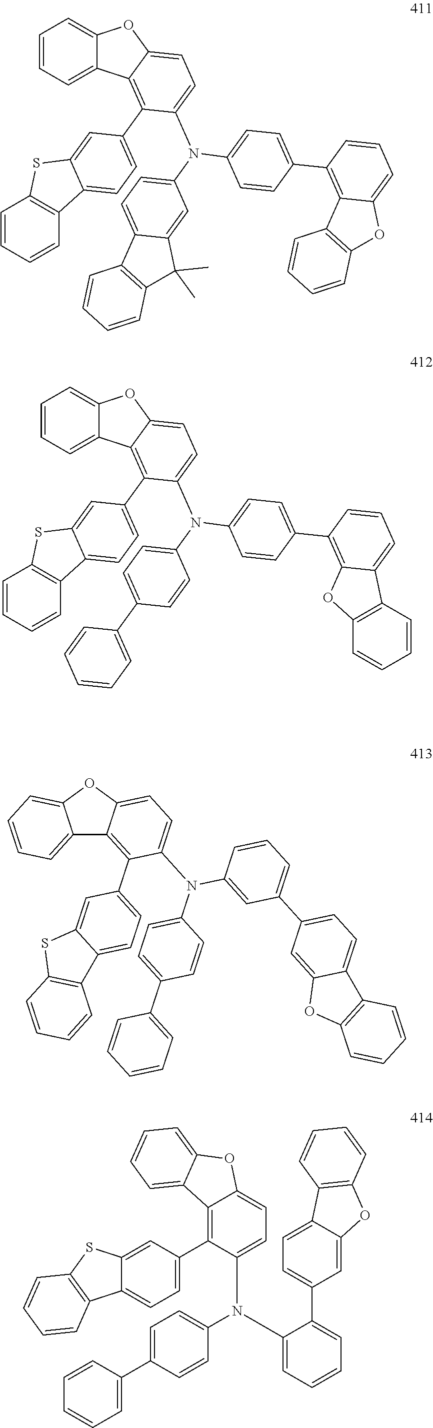 [SMILES: CC1(C)c2ccccc2-c2ccc(N(c3ccc(-c4cccc5oc6ccccc6c45)cc3)c3ccc4oc5ccccc5c4c3-c3ccc4c(c3)sc3ccccc34)cc21 ; c1ccc(-c2ccc(N(c3ccc(-c4cccc5c4oc4ccccc45)cc3)c3ccc4oc5ccccc5c4c3-c3ccc4c(c3)sc3ccccc34)cc2)cc1 ; c1ccc(-c2ccc(N(c3cccc(-c4ccc5c(c4)oc4ccccc45)c3)c3ccc4oc5ccccc5c4c3-c3ccc4c(c3)sc3ccccc34)cc2)cc1 ; c1ccc(-c2ccc(N(c3ccccc3-c3ccc4c(c3)oc3ccccc34)c3ccc4oc5ccccc5c4c3-c3ccc4c(c3)sc3ccccc34)cc2)cc1]

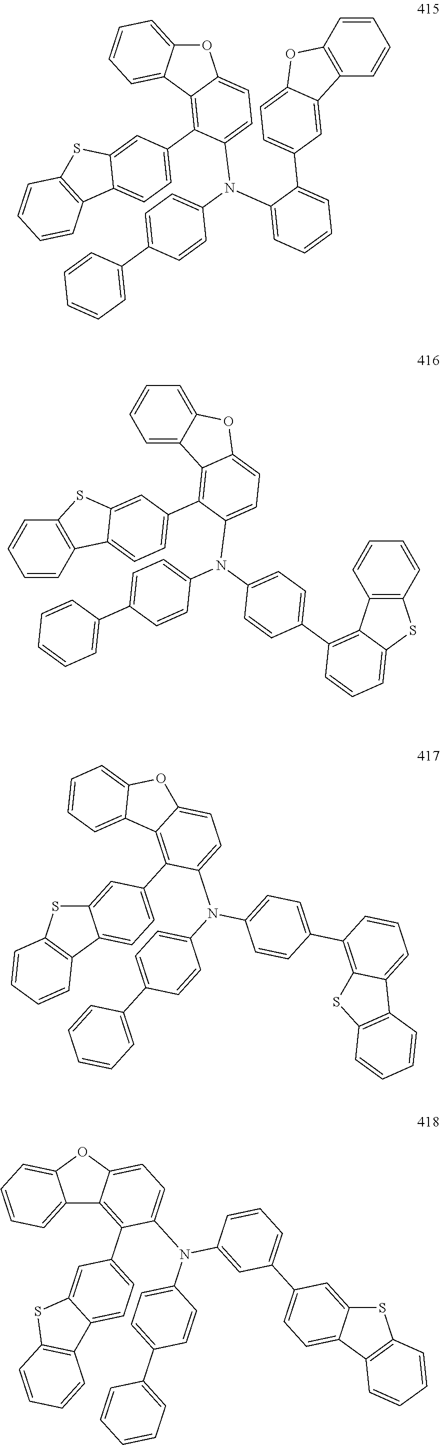 [SMILES: c1ccc(-c2ccc(N(c3ccccc3-c3ccc4oc5ccccc5c4c3)c3ccc4oc5ccccc5c4c3-c3ccc4c(c3)sc3ccccc34)cc2)cc1 ; c1ccc(-c2ccc(N(c3ccc(-c4cccc5sc6ccccc6c45)cc3)c3ccc4oc5ccccc5c4c3-c3ccc4c(c3)sc3ccccc34)cc2)cc1 ; c1ccc(-c2ccc(N(c3ccc(-c4cccc5c4sc4ccccc45)cc3)c3ccc4oc5ccccc5c4c3-c3ccc4c(c3)sc3ccccc34)cc2)cc1 ; c1ccc(-c2ccc(N(c3cccc(-c4ccc5c(c4)sc4ccccc45)c3)c3ccc4oc5ccccc5c4c3-c3ccc4c(c3)sc3ccccc34)cc2)cc1]

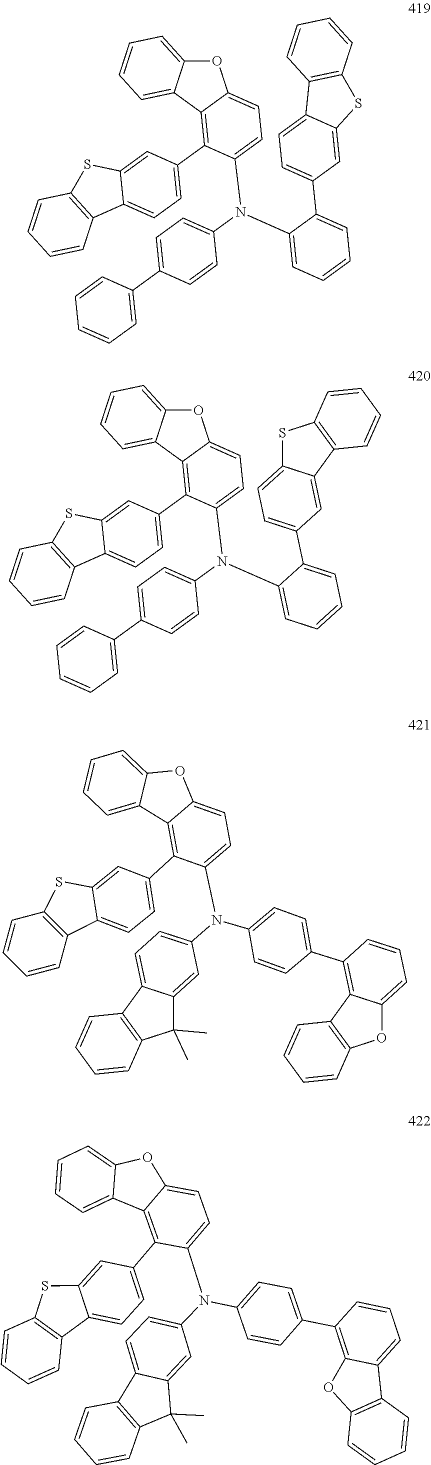 [SMILES: c1ccc(-c2ccc(N(c3ccccc3-c3ccc4c(c3)sc3ccccc34)c3ccc4oc5ccccc5c4c3-c3ccc4c(c3)sc3ccccc34)cc2)cc1 ; c1ccc(-c2ccc(N(c3ccccc3-c3ccc4sc5ccccc5c4c3)c3ccc4oc5ccccc5c4c3-c3ccc4c(c3)sc3ccccc34)cc2)cc1 ; CC1(C)c2ccccc2-c2ccc(N(c3ccc(-c4cccc5oc6ccccc6c45)cc3)c3ccc4oc5ccccc5c4c3-c3ccc4c(c3)sc3ccccc34)cc21 ; CC1(C)c2ccccc2-c2ccc(N(c3ccc(-c4cccc5c4oc4ccccc45)cc3)c3ccc4oc5ccccc5c4c3-c3ccc4c(c3)sc3ccccc34)cc21]

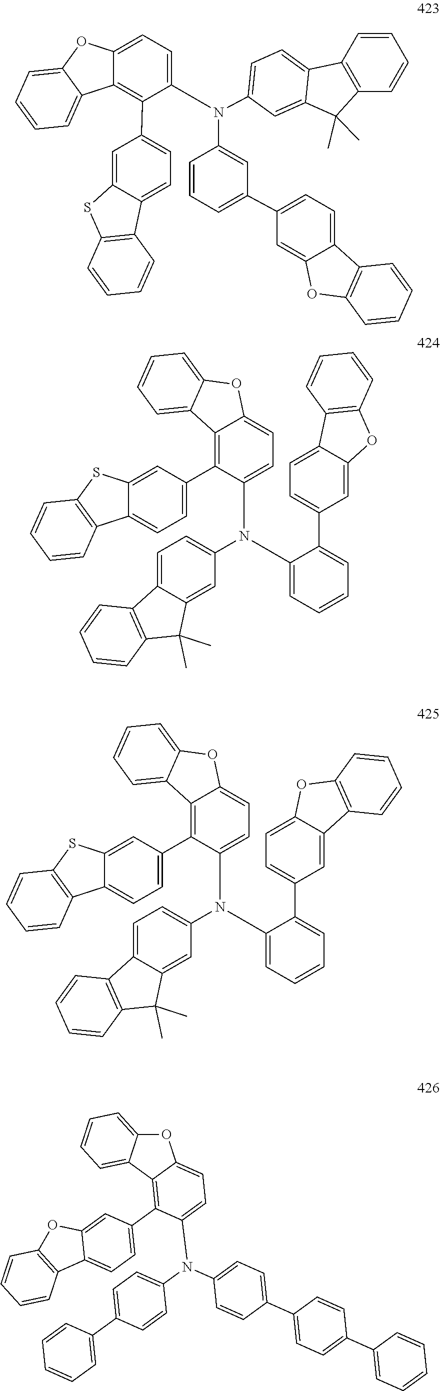

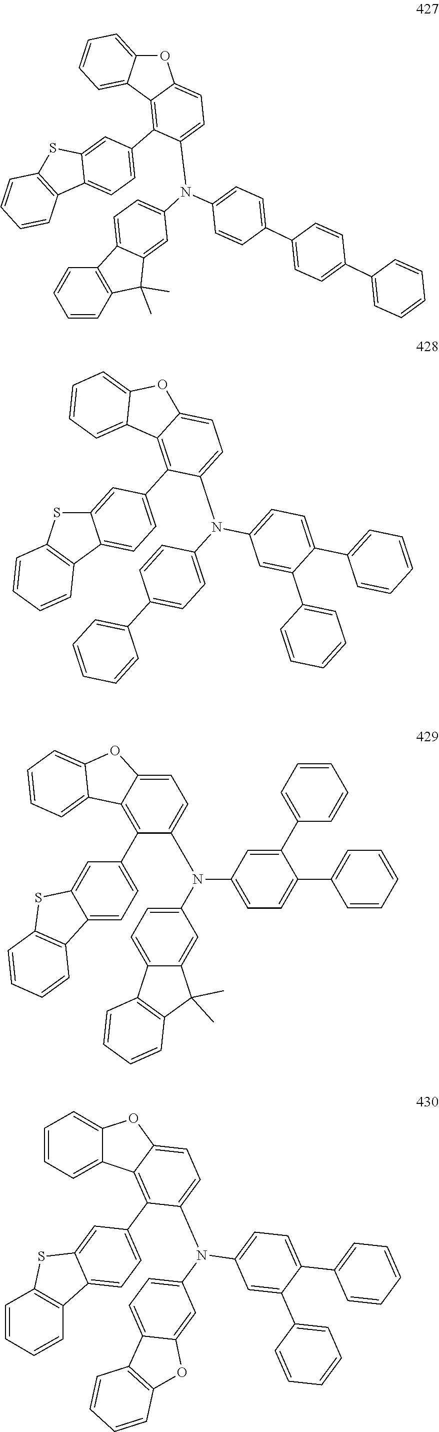 [SMILES: CC1(C)c2ccccc2-c2ccc(N(c3ccc(-c4ccc(-c5ccccc5)cc4)cc3)c3ccc4oc5ccccc5c4c3-c3ccc4c(c3)sc3ccccc34)cc21 ; c1ccc(-c2ccc(N(c3ccc(-c4ccccc4)c(-c4ccccc4)c3)c3ccc4oc5ccccc5c4c3-c3ccc4c(c3)sc3ccccc34)cc2)cc1 ; CC1(C)c2ccccc2-c2ccc(N(c3ccc(-c4ccccc4)c(-c4ccccc4)c3)c3ccc4oc5ccccc5c4c3-c3ccc4c(c3)sc3ccccc34)cc21 ; c1ccc(-c2ccc(N(c3ccc4c(c3)oc3ccccc34)c3ccc4oc5ccccc5c4c3-c3ccc4c(c3)sc3ccccc34)cc2-c2ccccc2)cc1]

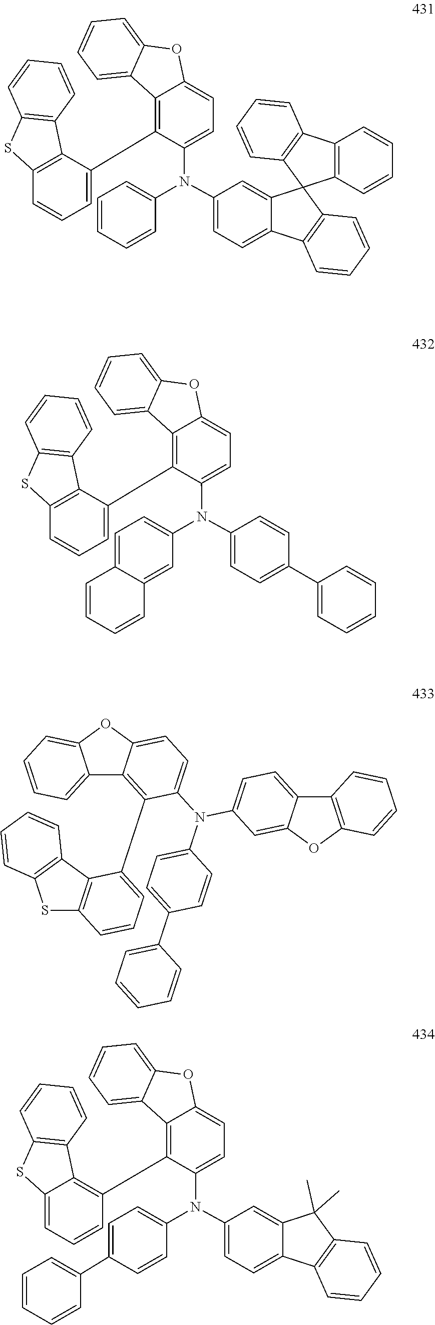

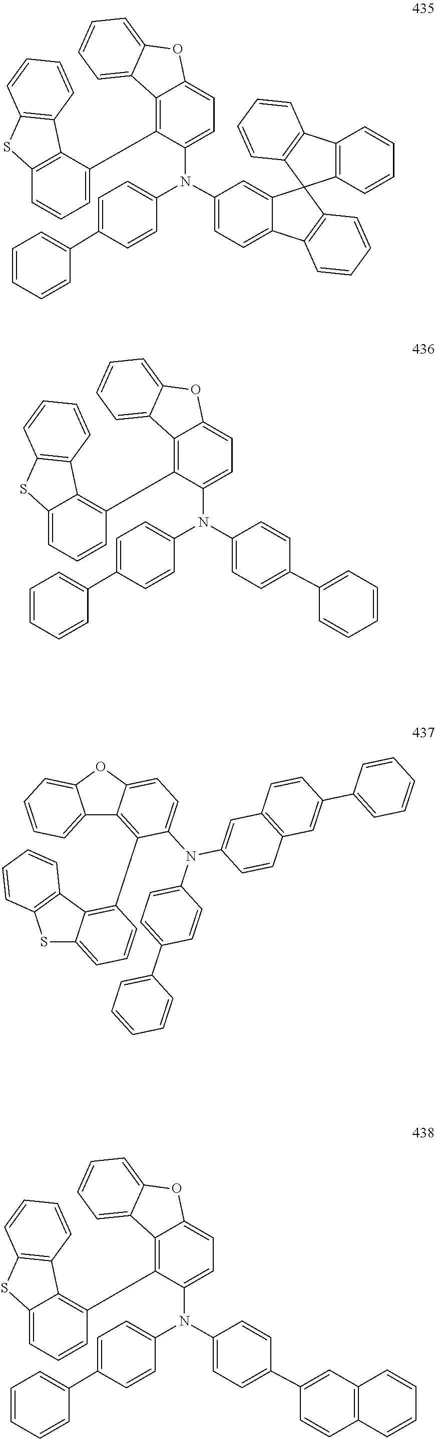 [SMILES: c1ccc(-c2ccc(N(c3ccc4c(c3)C3(c5ccccc5-c5ccccc53)c3ccccc3-4)c3ccc4oc5ccccc5c4c3-c3cccc4sc5ccccc5c34)cc2)cc1 ; c1ccc(-c2ccc(N(c3ccc(-c4ccccc4)cc3)c3ccc4oc5ccccc5c4c3-c3cccc4sc5ccccc5c34)cc2)cc1 ; c1ccc(-c2ccc(N(c3ccc4cc(-c5ccccc5)ccc4c3)c3ccc4oc5ccccc5c4c3-c3cccc4sc5ccccc5c34)cc2)cc1 ; c1ccc(-c2ccc(N(c3ccc(-c4ccc5ccccc5c4)cc3)c3ccc4oc5ccccc5c4c3-c3cccc4sc5ccccc5c34)cc2)cc1]

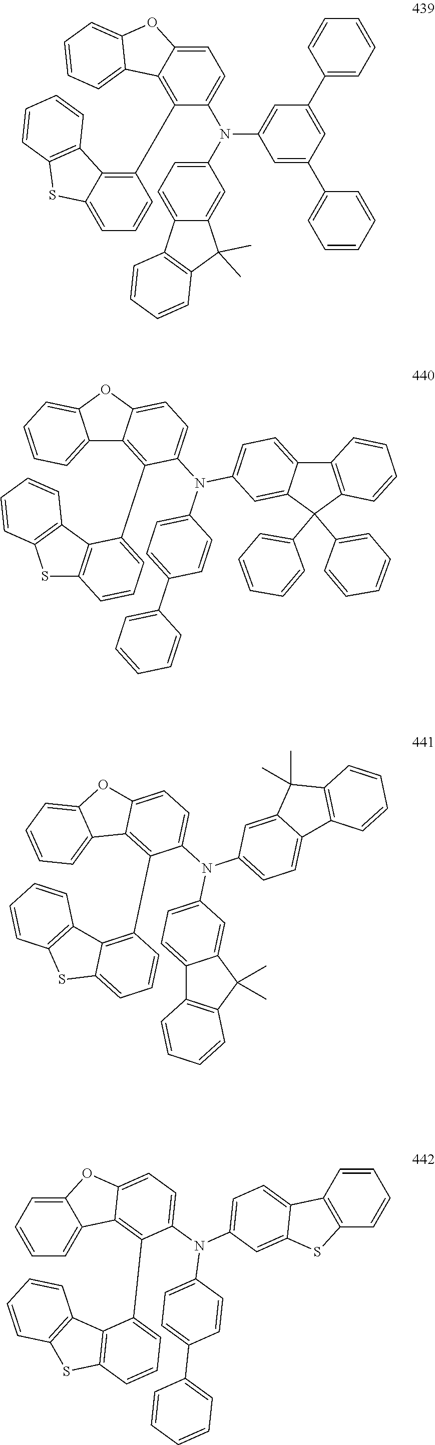 [SMILES: CC1(C)c2ccccc2-c2ccc(N(c3cc(-c4ccccc4)cc(-c4ccccc4)c3)c3ccc4oc5ccccc5c4c3-c3cccc4sc5ccccc5c34)cc21 ; c1ccc(-c2ccc(N(c3ccc4c(c3)C(c3ccccc3)(c3ccccc3)c3ccccc3-4)c3ccc4oc5ccccc5c4c3-c3cccc4sc5ccccc5c34)cc2)cc1 ; CC1(C)c2ccccc2-c2ccc(N(c3ccc4c(c3)C(C)(C)c3ccccc3-4)c3ccc4oc5ccccc5c4c3-c3cccc4sc5ccccc5c34)cc21 ; c1ccc(-c2ccc(N(c3ccc4c(c3)sc3ccccc34)c3ccc4oc5ccccc5c4c3-c3cccc4sc5ccccc5c34)cc2)cc1]

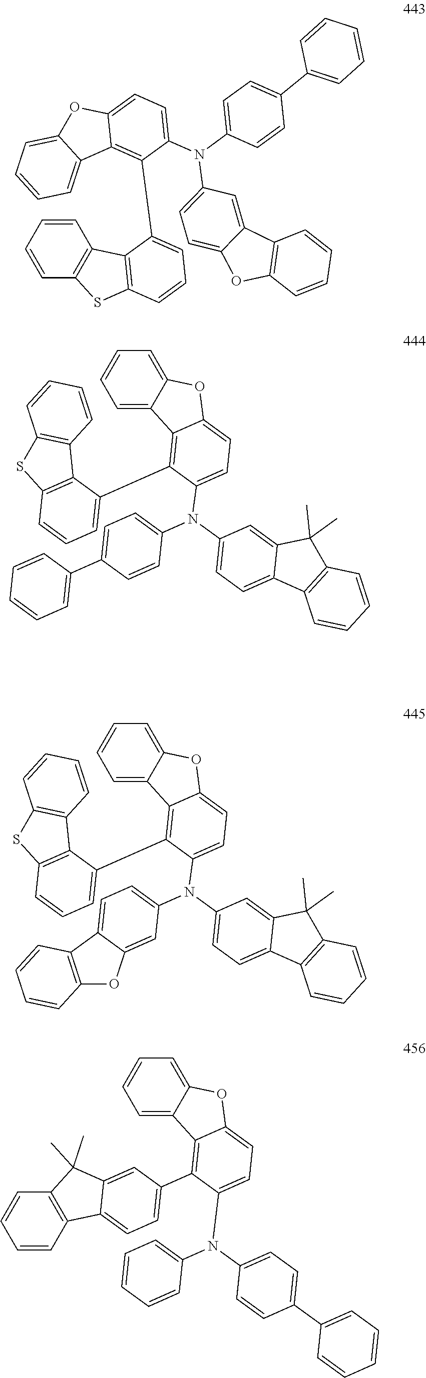 [SMILES: c1ccc(-c2ccc(N(c3ccc4oc5ccccc5c4c3)c3ccc4oc5ccccc5c4c3-c3cccc4sc5ccccc5c34)cc2)cc1 ; CC1(C)c2ccccc2-c2ccc(N(c3ccc(-c4ccccc4)cc3)c3ccc4oc5ccccc5c4c3-c3cccc4sc5ccccc5c34)cc21 ; CC1(C)c2ccccc2-c2ccc(N(c3ccc4c(c3)oc3ccccc34)c3ccc4oc5ccccc5c4c3-c3cccc4sc5ccccc5c34)cc21 ; CC1(C)c2ccccc2-c2ccc(-c3c(N(c4ccccc4)c4ccc(-c5ccccc5)cc4)ccc4oc5ccccc5c34)cc21]

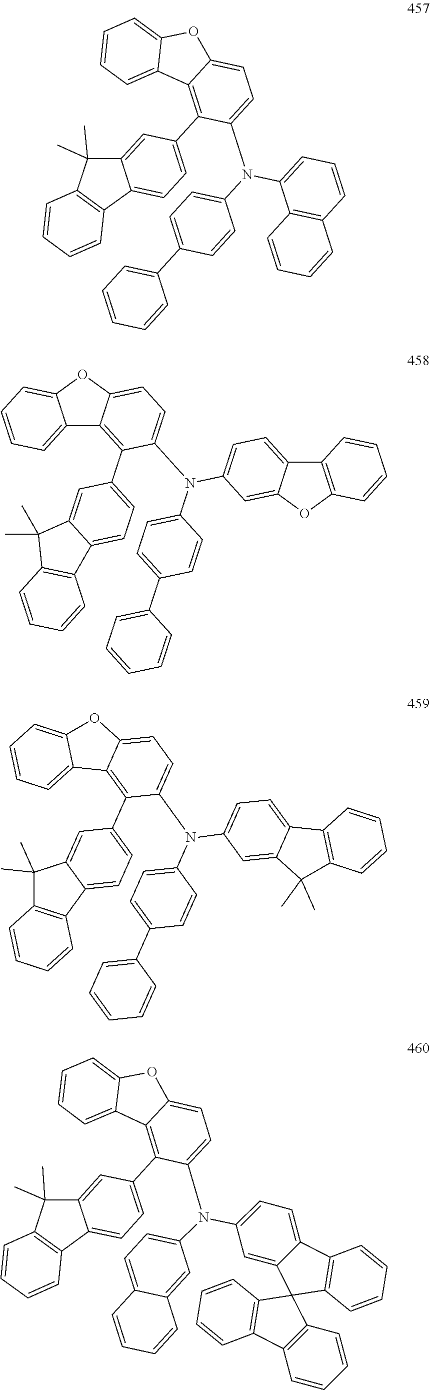

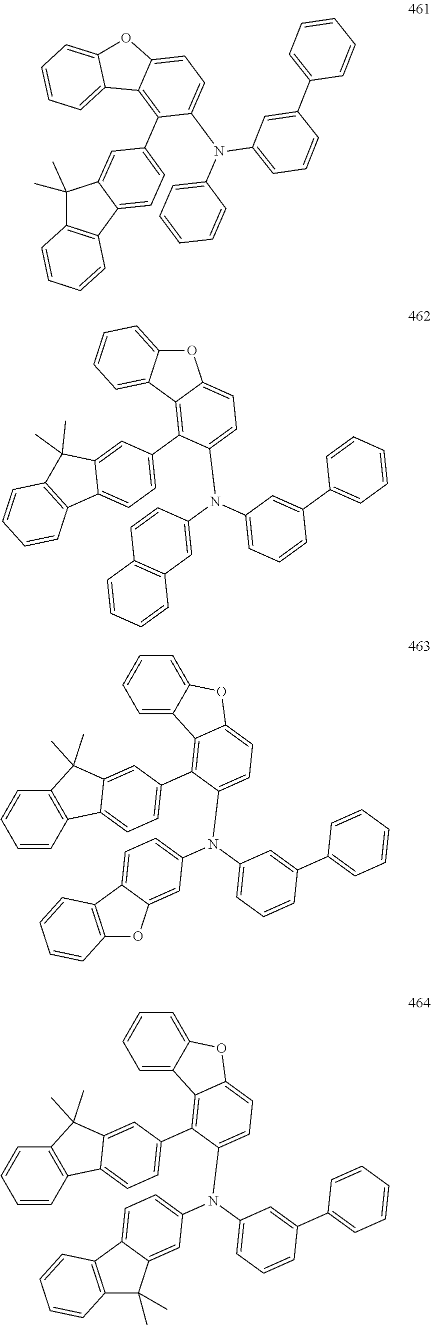

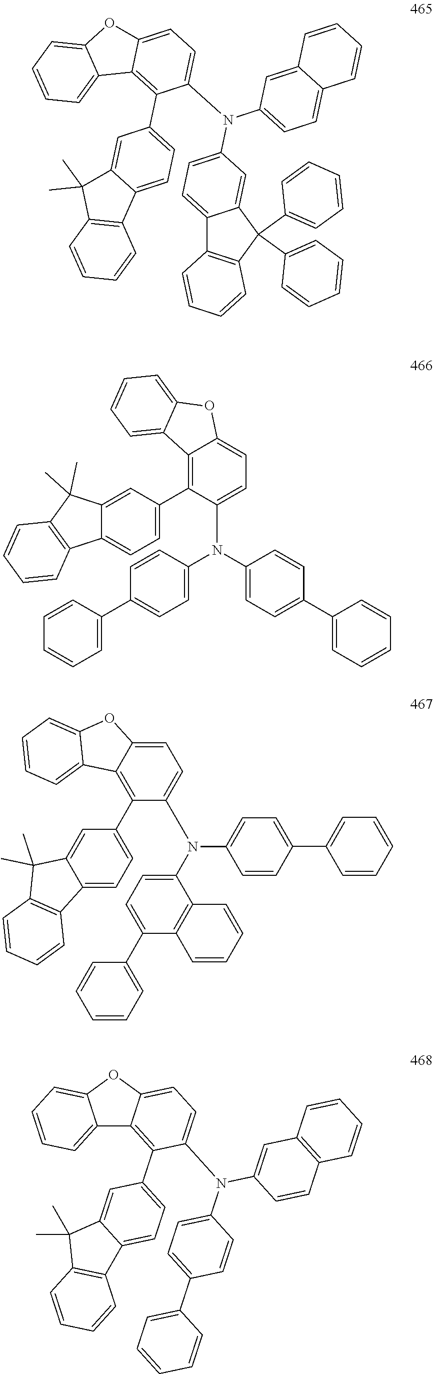 [SMILES: CC1(C)c2ccccc2-c2ccc(-c3c(N(c4ccc5c(c4)C(c4ccccc4)(c4ccccc4)c4ccccc4-5)c4ccc5ccccc5c4)ccc4oc5ccccc5c34)cc21 ; CC1(C)c2ccccc2-c2ccc(-c3c(N(c4ccc(-c5ccccc5)cc4)c4ccc(-c5ccccc5)cc4)ccc4oc5ccccc5c34)cc21 ; CC1(C)c2ccccc2-c2ccc(-c3c(N(c4ccc(-c5ccccc5)cc4)c4ccc(-c5ccccc5)c5ccccc45)ccc4oc5ccccc5c34)cc21 ; CC1(C)c2ccccc2-c2ccc(-c3c(N(c4ccc(-c5ccccc5)cc4)c4ccc5ccccc5c4)ccc4oc5ccccc5c34)cc21]

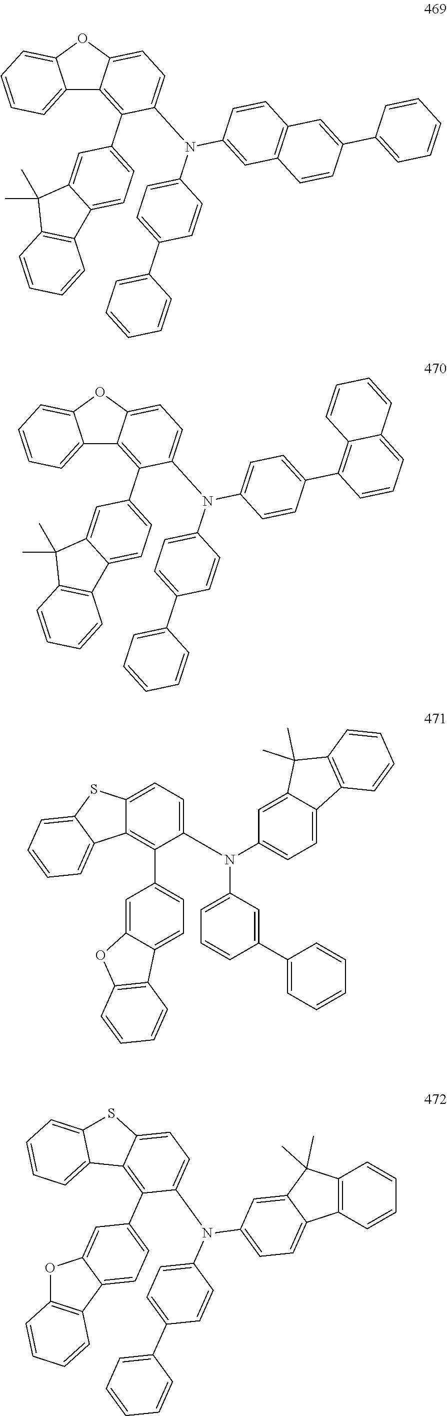 [SMILES: CC1(C)c2ccccc2-c2ccc(-c3c(N(c4ccc(-c5ccccc5)cc4)c4ccc5cc(-c6ccccc6)ccc5c4)ccc4oc5ccccc5c34)cc21 ; CC1(C)c2ccccc2-c2ccc(-c3c(N(c4ccc(-c5ccccc5)cc4)c4ccc(-c5cccc6ccccc56)cc4)ccc4oc5ccccc5c34)cc21 ; CC1(C)c2ccccc2-c2ccc(N(c3cccc(-c4ccccc4)c3)c3ccc4sc5ccccc5c4c3-c3ccc4c(c3)oc3ccccc34)cc21 ; CC1(C)c2ccccc2-c2ccc(N(c3ccc(-c4ccccc4)cc3)c3ccc4sc5ccccc5c4c3-c3ccc4c(c3)oc3ccccc34)cc21]

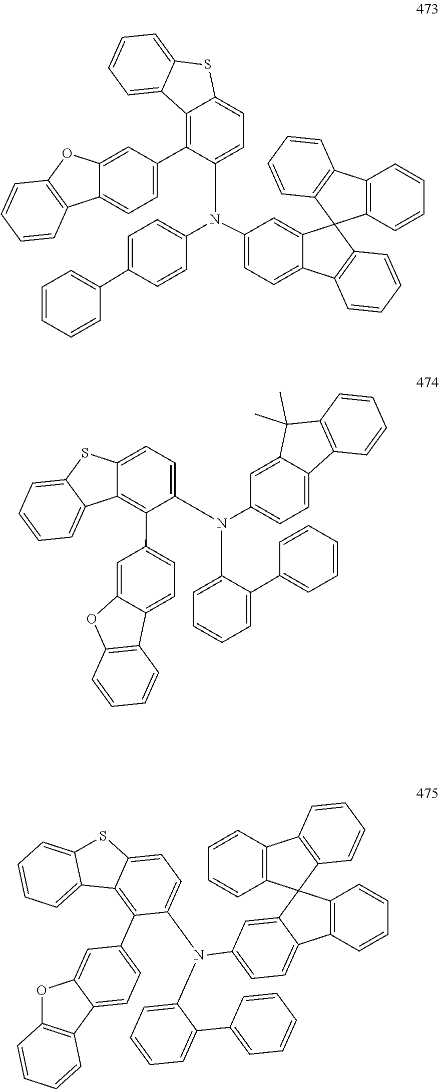 [SMILES: c1ccc(-c2ccc(N(c3ccc4c(c3)C3(c5ccccc5-c5ccccc53)c3ccccc3-4)c3ccc4sc5ccccc5c4c3-c3ccc4c(c3)oc3ccccc34)cc2)cc1 ; CC1(C)c2ccccc2-c2ccc(N(c3ccccc3-c3ccccc3)c3ccc4sc5ccccc5c4c3-c3ccc4c(c3)oc3ccccc34)cc21 ; c1ccc(-c2ccccc2N(c2ccc3c(c2)C2(c4ccccc4-c4ccccc42)c2ccccc2-3)c2ccc3sc4ccccc4c3c2-c2ccc3c(c2)oc2ccccc23)cc1]

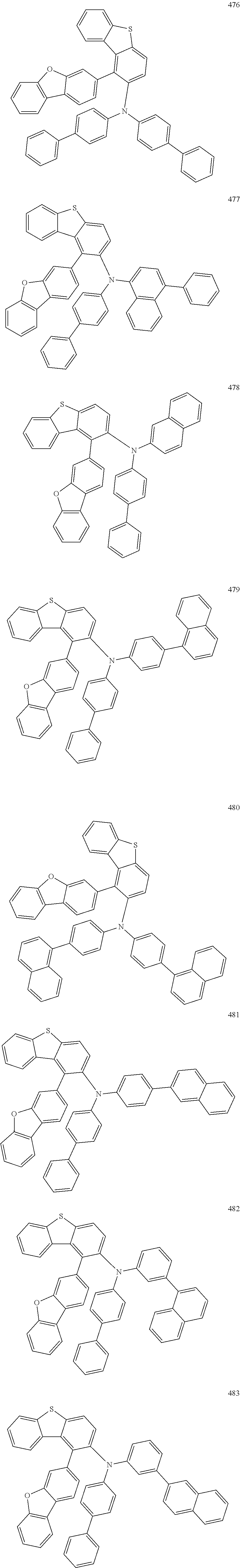

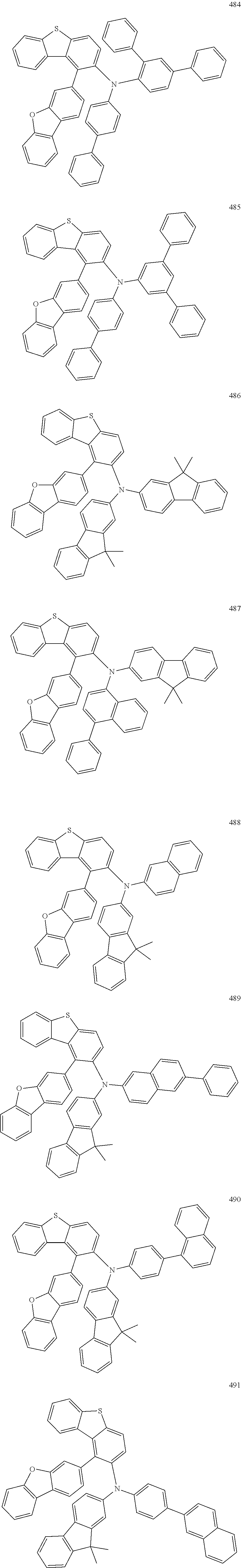

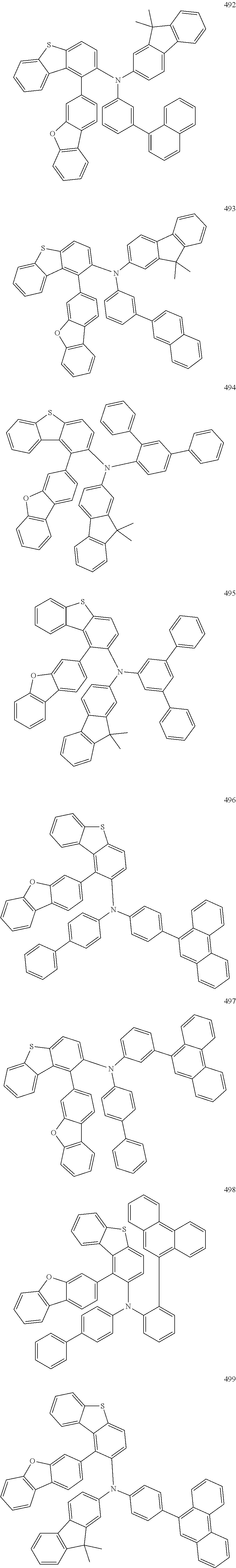

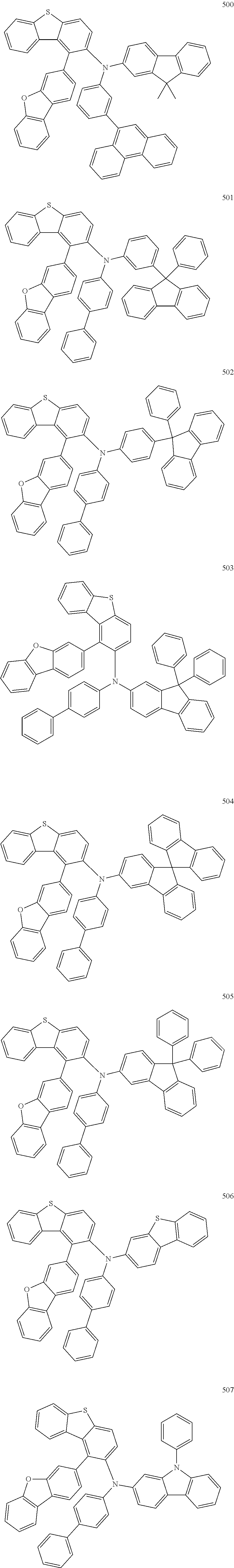

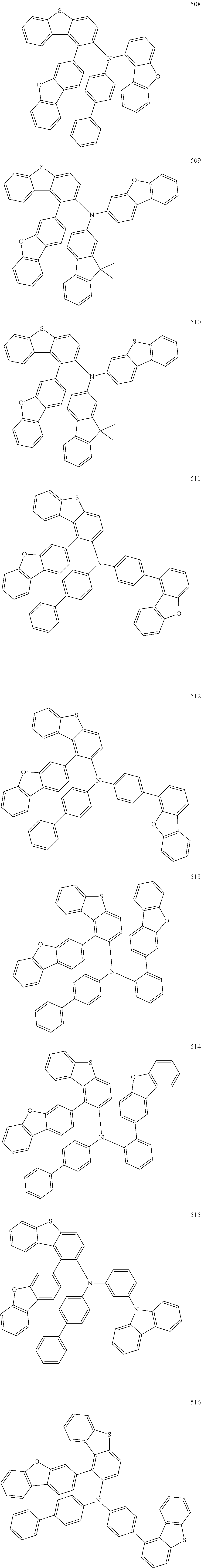

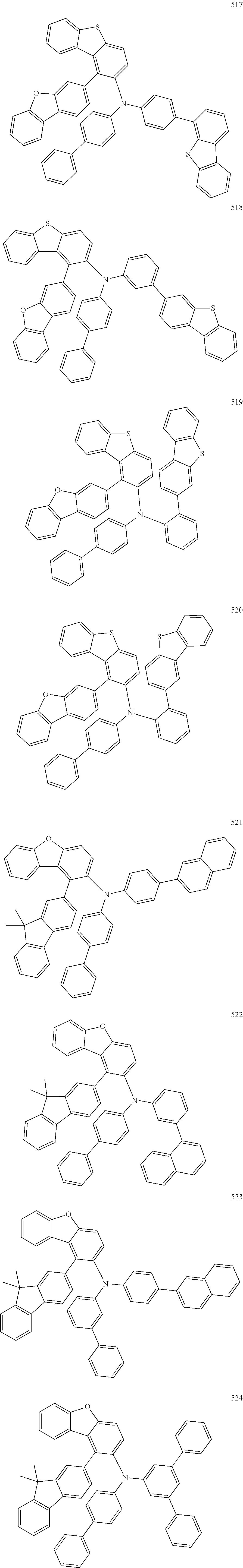

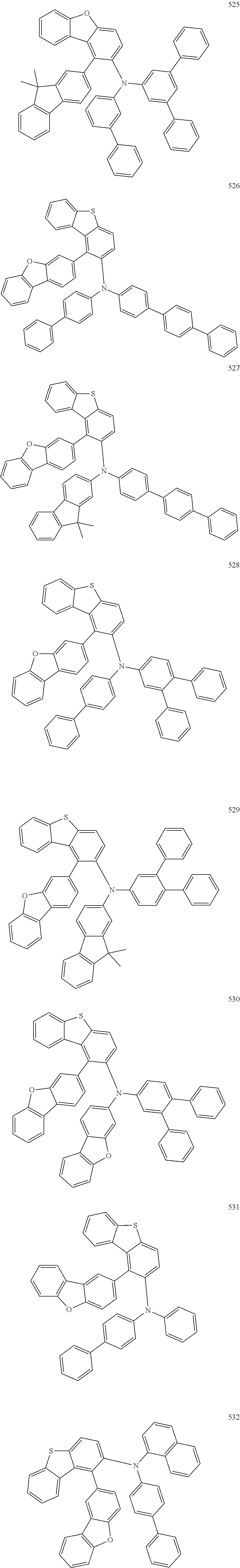

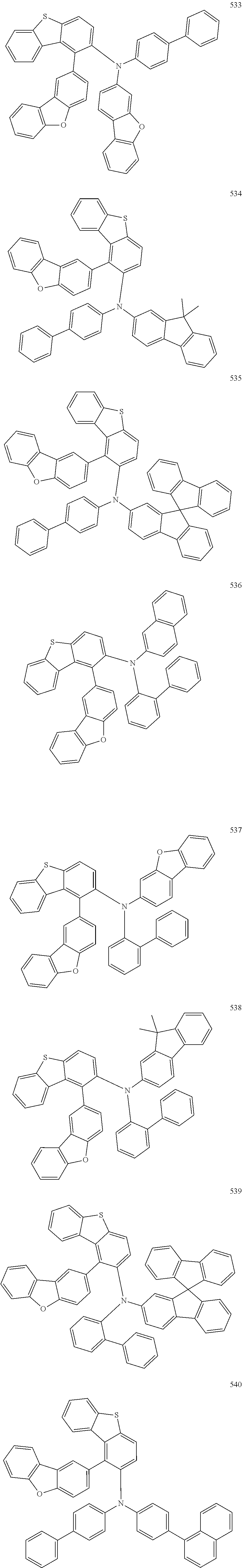

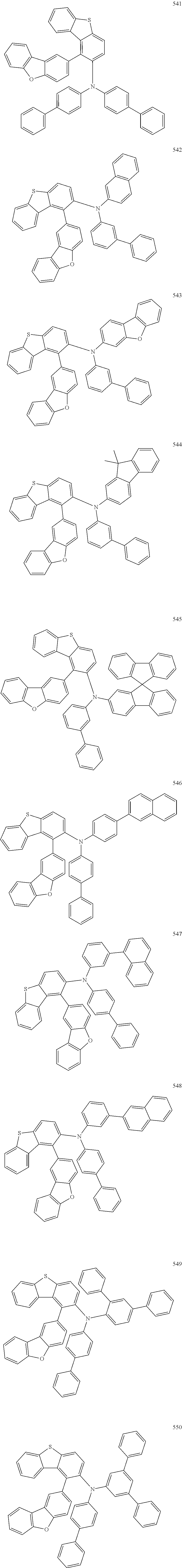

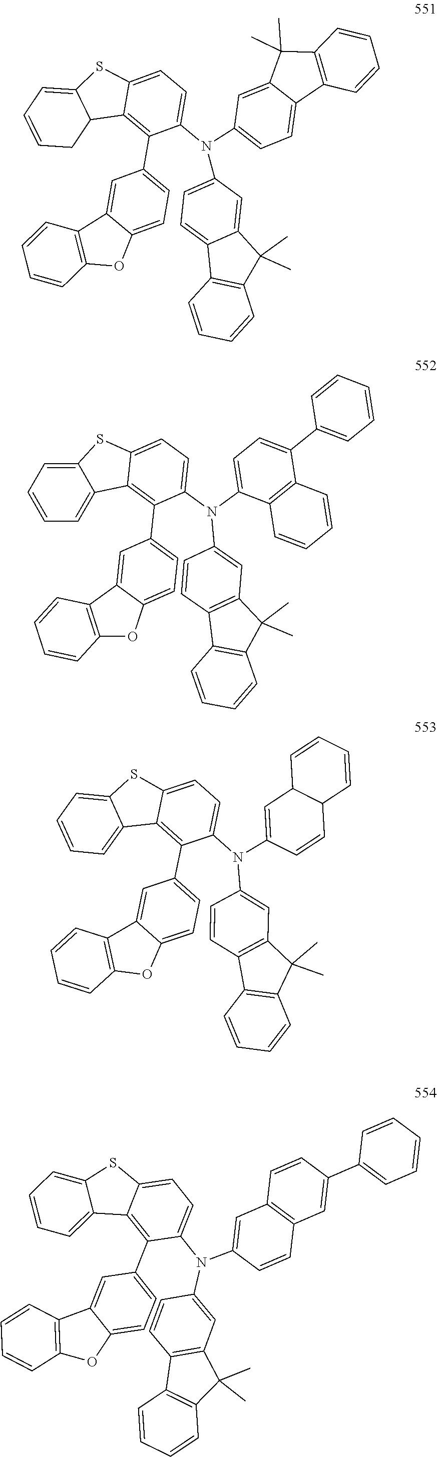 [SMILES: CC1(C)c2ccccc2-c2ccc(N(c3ccc4c(c3)C(C)(C)c3ccccc3-4)c3ccc4c(c3-c3ccc5oc6ccccc6c5c3)C3CC=CC=C3S4)cc21 ; CC1(C)c2ccccc2-c2ccc(N(c3ccc4sc5ccccc5c4c3-c3ccc4oc5ccccc5c4c3)c3ccc(-c4ccccc4)c4ccccc34)cc21 ; CC1(C)c2ccccc2-c2ccc(N(C3=CC4C=CC=CC4C=C3)c3ccc4sc5ccccc5c4c3-c3ccc4oc5ccccc5c4c3)cc21 ; CC1(C)c2ccccc2-c2ccc(N(c3ccc4cc(-c5ccccc5)ccc4c3)c3ccc4sc5ccccc5c4c3-c3ccc4oc5ccccc5c4c3)cc21]

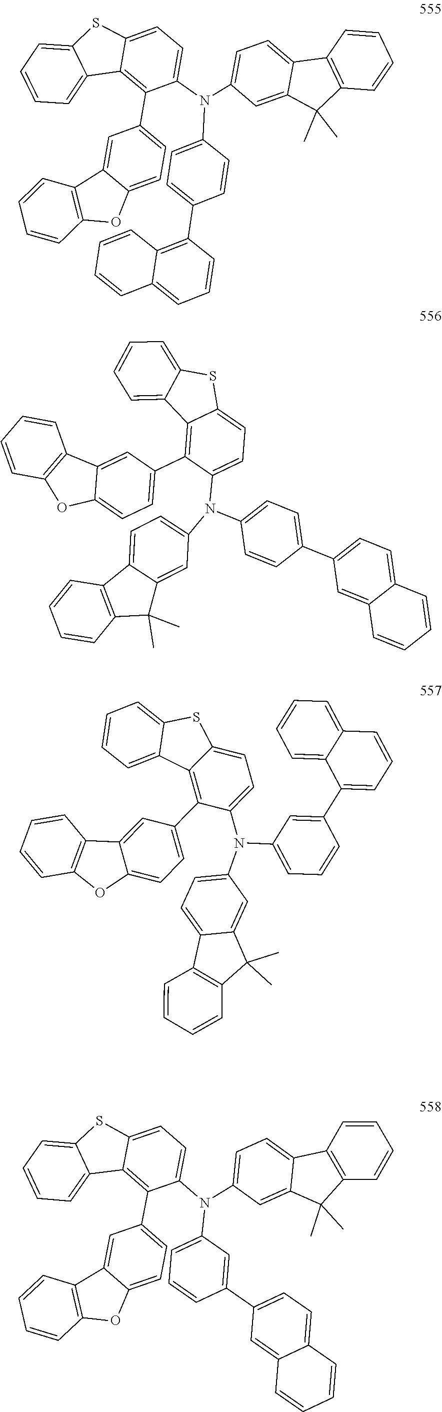

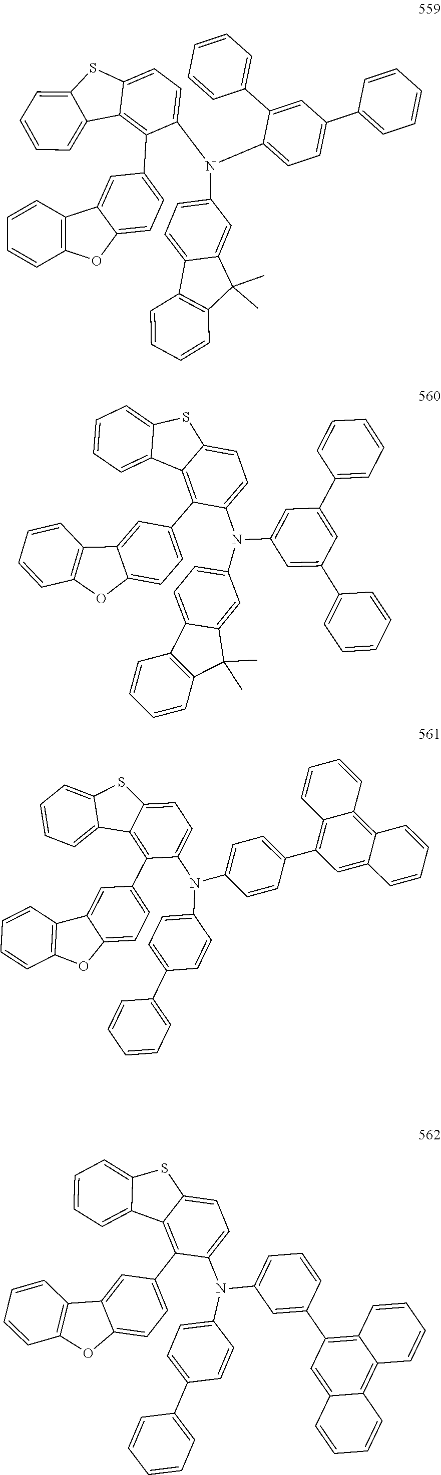 [SMILES: CC1(C)c2ccccc2-c2ccc(N(c3ccc(-c4ccccc4)cc3-c3ccccc3)c3ccc4sc5ccccc5c4c3-c3ccc4oc5ccccc5c4c3)cc21 ; CC1(C)c2ccccc2-c2ccc(N(c3cc(-c4ccccc4)cc(-c4ccccc4)c3)c3ccc4sc5ccccc5c4c3-c3ccc4oc5ccccc5c4c3)cc21 ; c1ccc(-c2ccc(N(c3ccc(-c4cc5ccccc5c5ccccc45)cc3)c3ccc4sc5ccccc5c4c3-c3ccc4oc5ccccc5c4c3)cc2)cc1 ; c1ccc(-c2ccc(N(c3cccc(-c4cc5ccccc5c5ccccc45)c3)c3ccc4sc5ccccc5c4c3-c3ccc4oc5ccccc5c4c3)cc2)cc1]

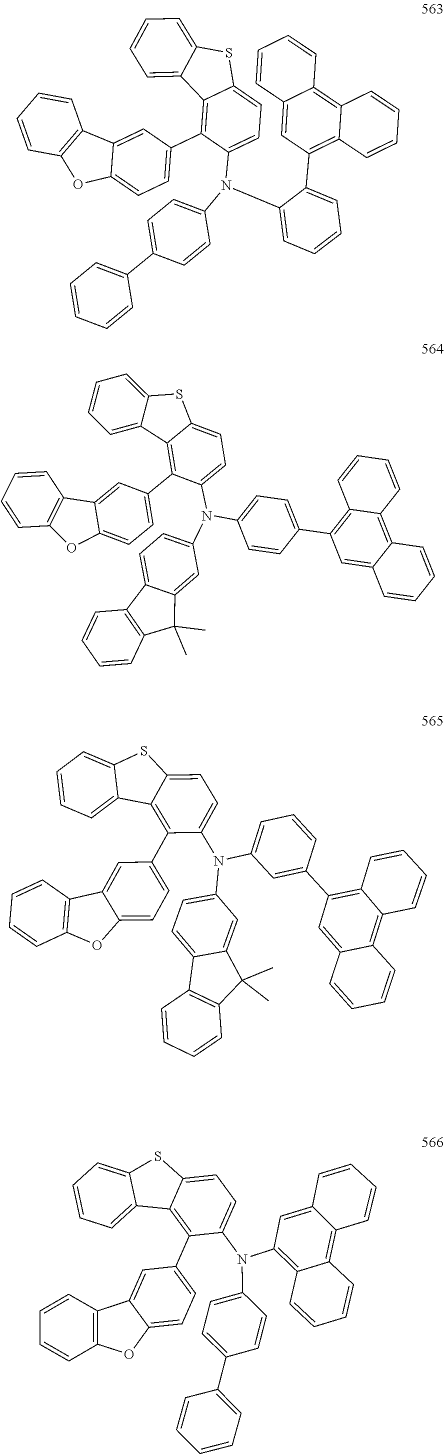 [SMILES: c1ccc(-c2ccc(N(c3ccccc3-c3cc4ccccc4c4ccccc34)c3ccc4sc5ccccc5c4c3-c3ccc4oc5ccccc5c4c3)cc2)cc1 ; CC1(C)c2ccccc2-c2ccc(N(c3ccc(-c4cc5ccccc5c5ccccc45)cc3)c3ccc4sc5ccccc5c4c3-c3ccc4oc5ccccc5c4c3)cc21 ; CC1(C)c2ccccc2-c2ccc(N(c3cccc(-c4cc5ccccc5c5ccccc45)c3)c3ccc4sc5ccccc5c4c3-c3ccc4oc5ccccc5c4c3)cc21 ; c1ccc(-c2ccc(N(c3ccc4sc5ccccc5c4c3-c3ccc4oc5ccccc5c4c3)c3cc4ccccc4c4ccccc34)cc2)cc1]

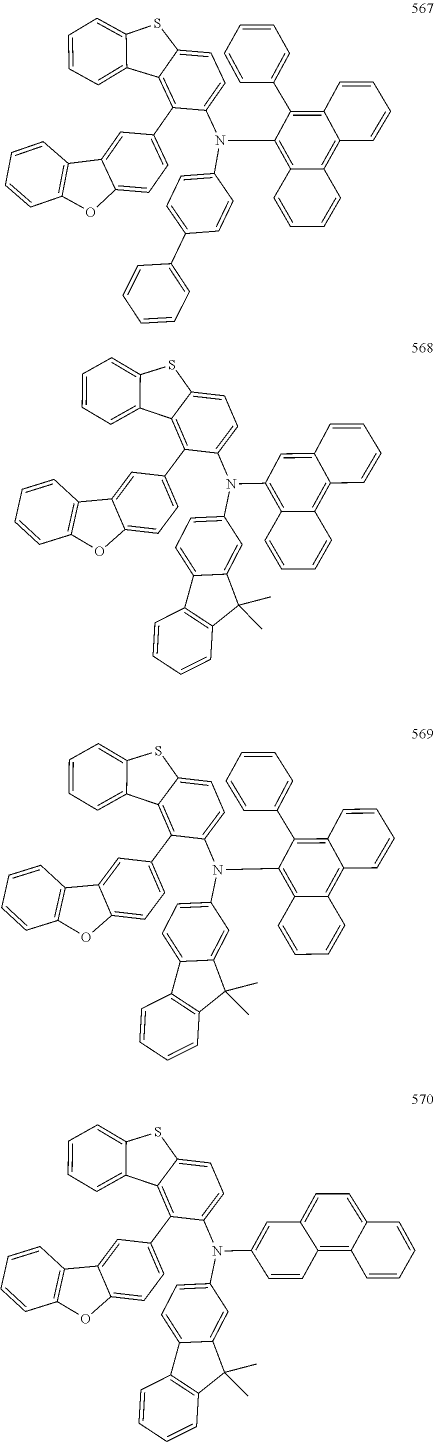

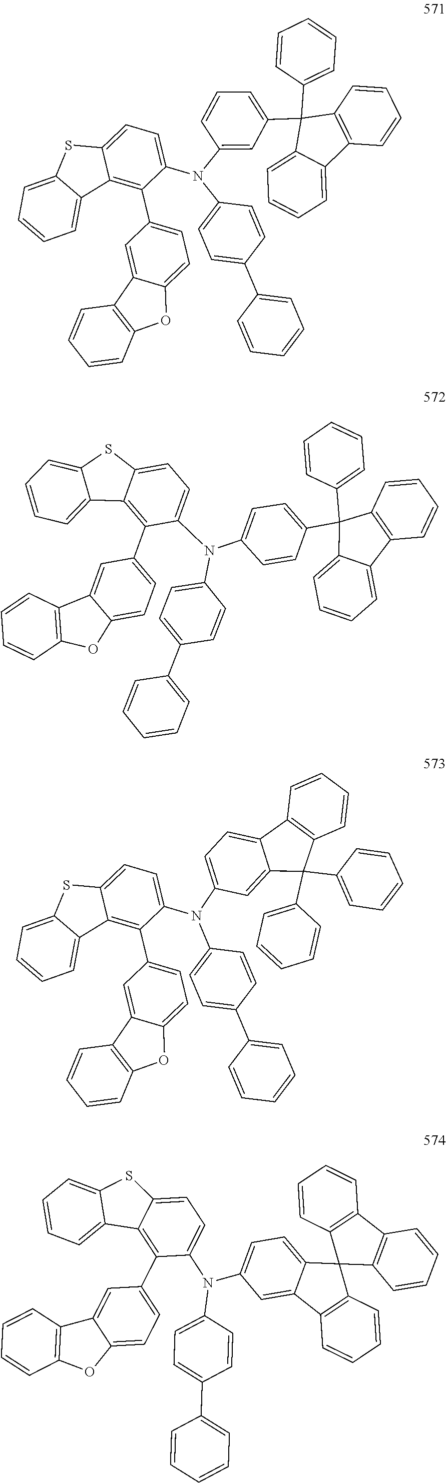 [SMILES: c1ccc(-c2ccc(N(c3cccc(C4(c5ccccc5)c5ccccc5-c5ccccc54)c3)c3ccc4sc5ccccc5c4c3-c3ccc4oc5ccccc5c4c3)cc2)cc1 ; c1ccc(-c2ccc(N(c3ccc(C4(c5ccccc5)c5ccccc5-c5ccccc54)cc3)c3ccc4sc5ccccc5c4c3-c3ccc4oc5ccccc5c4c3)cc2)cc1 ; c1ccc(-c2ccc(N(c3ccc4c(c3)C(c3ccccc3)(c3ccccc3)c3ccccc3-4)c3ccc4sc5ccccc5c4c3-c3ccc4oc5ccccc5c4c3)cc2)cc1 ; c1ccc(-c2ccc(N(c3ccc4c(c3)-c3ccccc3C43c4ccccc4-c4ccccc43)c3ccc4sc5ccccc5c4c3-c3ccc4oc5ccccc5c4c3)cc2)cc1]

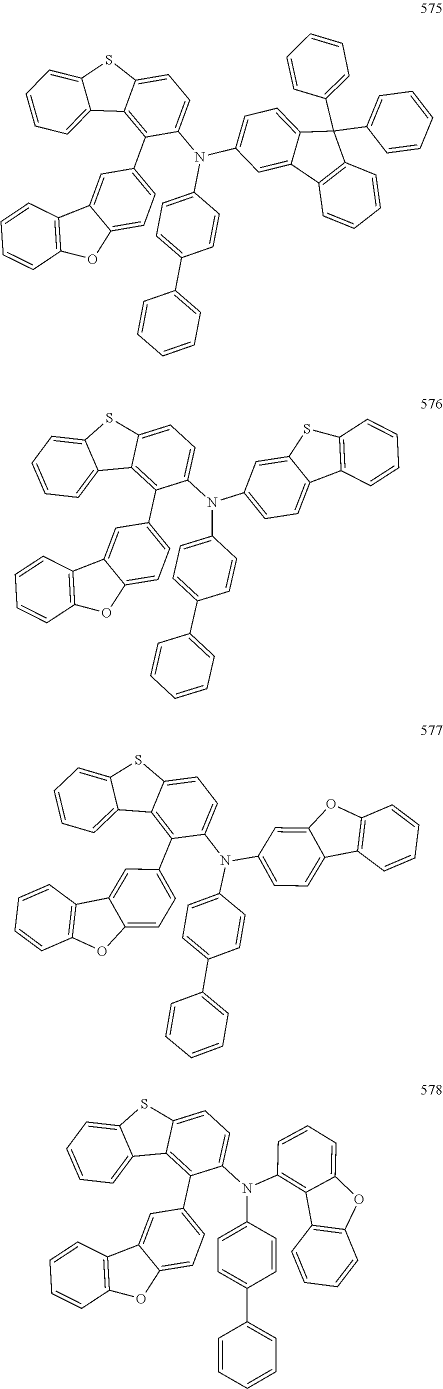 [SMILES: c1ccc(-c2ccc(N(c3ccc4c(c3)-c3ccccc3C4(c3ccccc3)c3ccccc3)c3ccc4sc5ccccc5c4c3-c3ccc4oc5ccccc5c4c3)cc2)cc1 ; c1ccc(-c2ccc(N(c3ccc4c(c3)sc3ccccc34)c3ccc4sc5ccccc5c4c3-c3ccc4oc5ccccc5c4c3)cc2)cc1 ; c1ccc(-c2ccc(N(c3ccc4c(c3)oc3ccccc34)c3ccc4sc5ccccc5c4c3-c3ccc4oc5ccccc5c4c3)cc2)cc1 ; c1ccc(-c2ccc(N(c3ccc4sc5ccccc5c4c3-c3ccc4oc5ccccc5c4c3)c3cccc4oc5ccccc5c34)cc2)cc1]

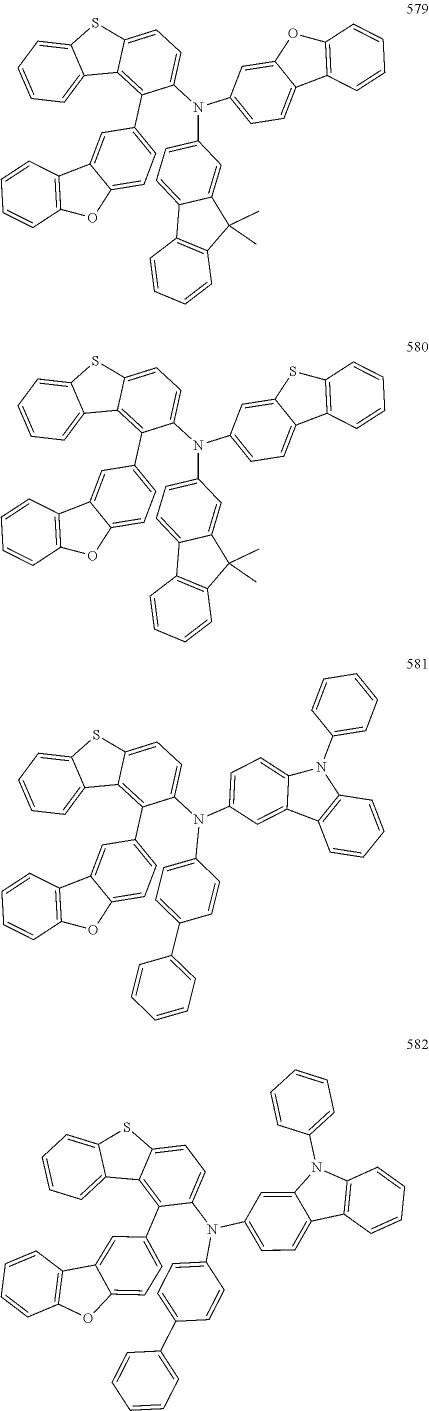 [SMILES: CC1(C)c2ccccc2-c2ccc(N(c3ccc4c(c3)oc3ccccc34)c3ccc4sc5ccccc5c4c3-c3ccc4oc5ccccc5c4c3)cc21 ; CC1(C)c2ccccc2-c2ccc(N(c3ccc4c(c3)sc3ccccc34)c3ccc4sc5ccccc5c4c3-c3ccc4oc5ccccc5c4c3)cc21 ; c1ccc(-c2ccc(N(c3ccc4c(c3)c3ccccc3n4-c3ccccc3)c3ccc4sc5ccccc5c4c3-c3ccc4oc5ccccc5c4c3)cc2)cc1 ; c1ccc(-c2ccc(N(c3ccc4c5ccccc5n(-c5ccccc5)c4c3)c3ccc4sc5ccccc5c4c3-c3ccc4oc5ccccc5c4c3)cc2)cc1]

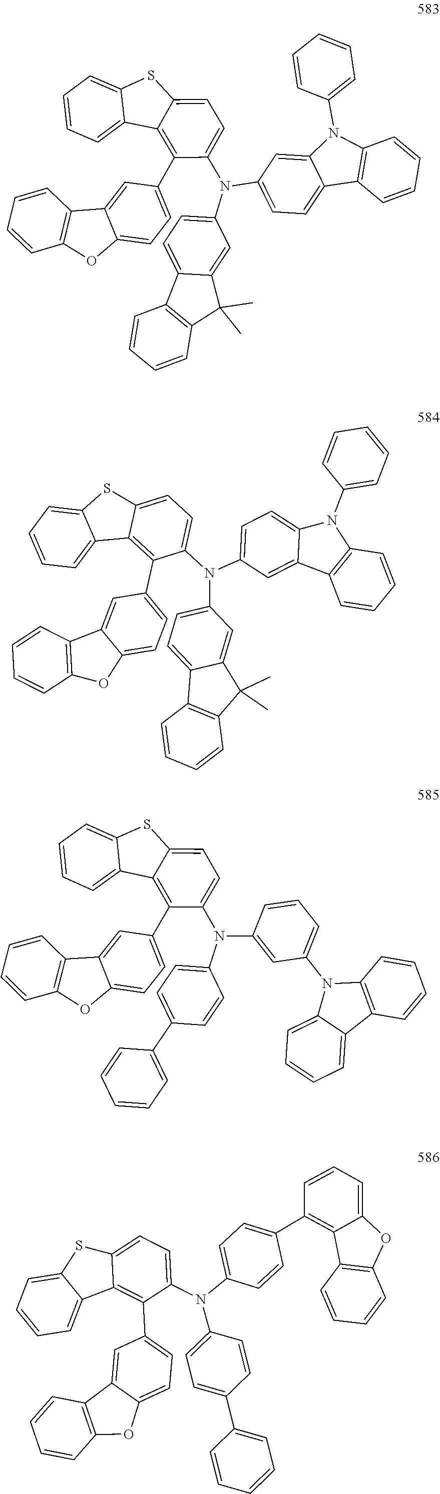 [SMILES: CC1(C)c2ccccc2-c2ccc(N(c3ccc4c5ccccc5n(-c5ccccc5)c4c3)c3ccc4sc5ccccc5c4c3-c3ccc4oc5ccccc5c4c3)cc21 ; CC1(C)c2ccccc2-c2ccc(N(c3ccc4c(c3)c3ccccc3n4-c3ccccc3)c3ccc4sc5ccccc5c4c3-c3ccc4oc5ccccc5c4c3)cc21 ; c1ccc(-c2ccc(N(c3cccc(-n4c5ccccc5c5ccccc54)c3)c3ccc4sc5ccccc5c4c3-c3ccc4oc5ccccc5c4c3)cc2)cc1 ; c1ccc(-c2ccc(N(c3ccc(-c4cccc5oc6ccccc6c45)cc3)c3ccc4sc5ccccc5c4c3-c3ccc4oc5ccccc5c4c3)cc2)cc1]

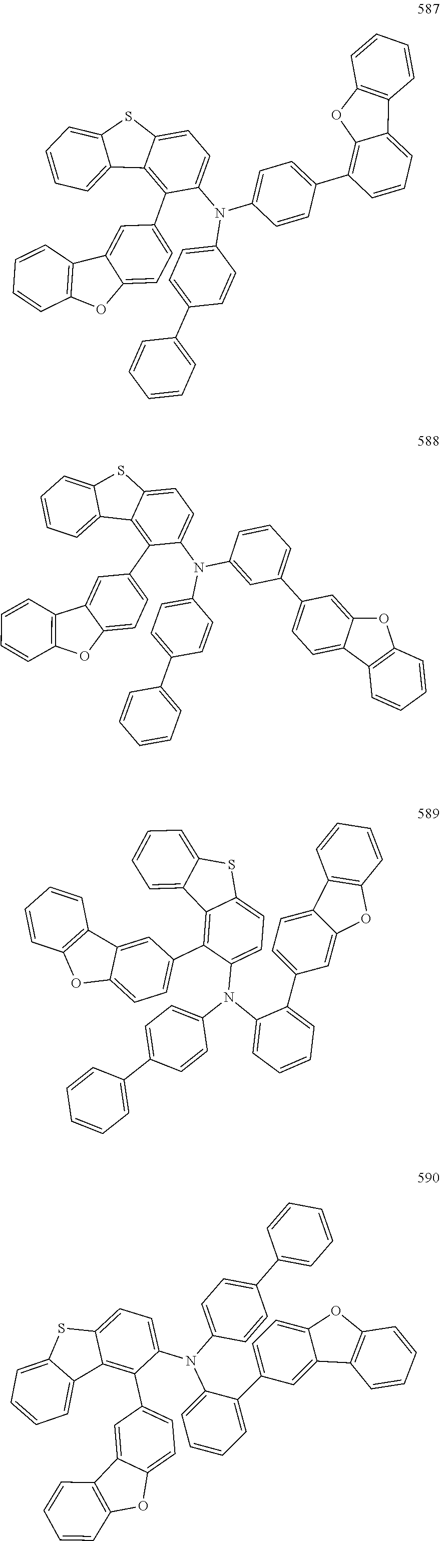 [SMILES: c1ccc(-c2ccc(N(c3ccc(-c4cccc5c4oc4ccccc45)cc3)c3ccc4sc5ccccc5c4c3-c3ccc4oc5ccccc5c4c3)cc2)cc1 ; c1ccc(-c2ccc(N(c3cccc(-c4ccc5c(c4)oc4ccccc45)c3)c3ccc4sc5ccccc5c4c3-c3ccc4oc5ccccc5c4c3)cc2)cc1 ; c1ccc(-c2ccc(N(c3ccccc3-c3ccc4c(c3)oc3ccccc34)c3ccc4sc5ccccc5c4c3-c3ccc4oc5ccccc5c4c3)cc2)cc1 ; c1ccc(-c2ccc(N(c3ccccc3-c3ccc4oc5ccccc5c4c3)c3ccc4sc5ccccc5c4c3-c3ccc4oc5ccccc5c4c3)cc2)cc1]

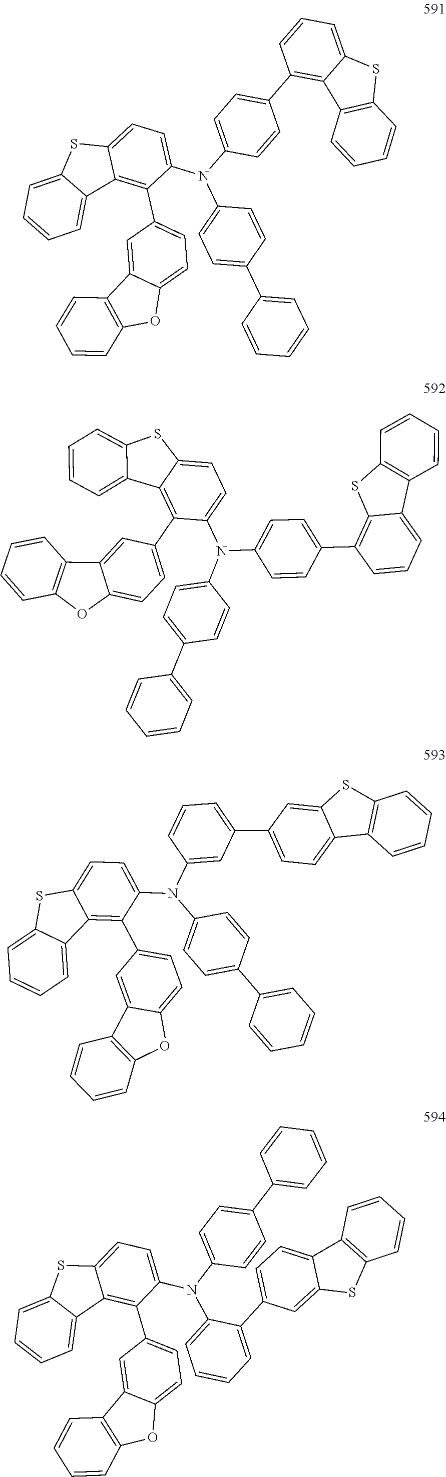 [SMILES: c1ccc(-c2ccc(N(c3ccc(-c4cccc5sc6ccccc6c45)cc3)c3ccc4sc5ccccc5c4c3-c3ccc4oc5ccccc5c4c3)cc2)cc1 ; c1ccc(-c2ccc(N(c3ccc(-c4cccc5c4sc4ccccc45)cc3)c3ccc4sc5ccccc5c4c3-c3ccc4oc5ccccc5c4c3)cc2)cc1 ; c1ccc(-c2ccc(N(c3cccc(-c4ccc5c(c4)sc4ccccc45)c3)c3ccc4sc5ccccc5c4c3-c3ccc4oc5ccccc5c4c3)cc2)cc1 ; c1ccc(-c2ccc(N(c3ccccc3-c3ccc4c(c3)sc3ccccc34)c3ccc4sc5ccccc5c4c3-c3ccc4oc5ccccc5c4c3)cc2)cc1]

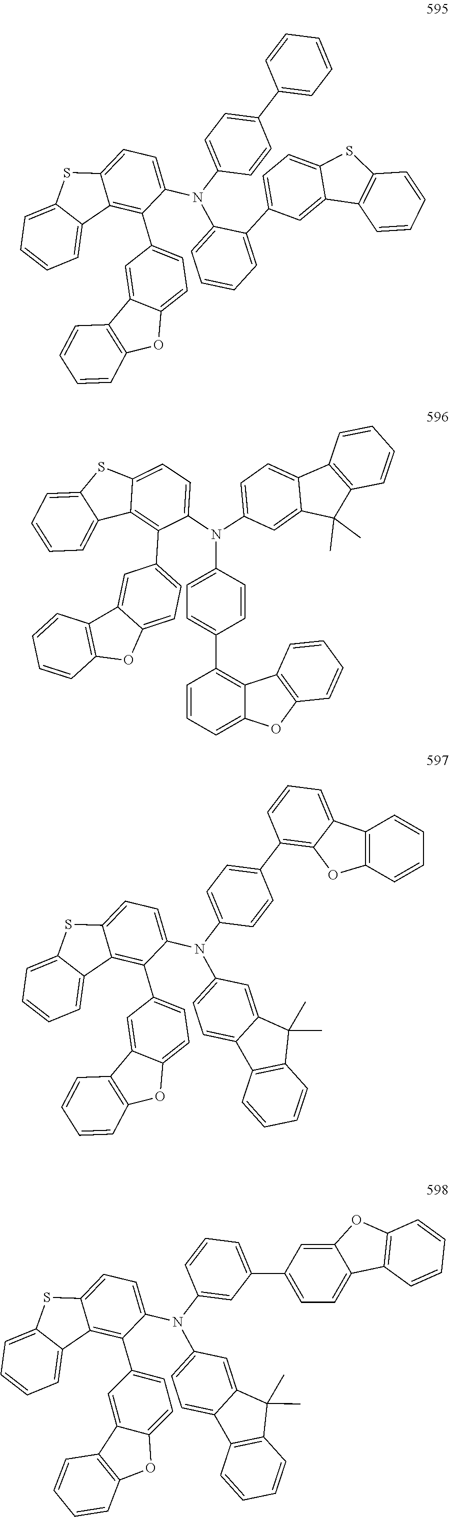 [SMILES: c1ccc(-c2ccc(N(c3ccccc3-c3ccc4sc5ccccc5c4c3)c3ccc4sc5ccccc5c4c3-c3ccc4oc5ccccc5c4c3)cc2)cc1 ; CC1(C)c2ccccc2-c2ccc(N(c3ccc(-c4cccc5oc6ccccc6c45)cc3)c3ccc4sc5ccccc5c4c3-c3ccc4oc5ccccc5c4c3)cc21 ; CC1(C)c2ccccc2-c2ccc(N(c3ccc(-c4cccc5c4oc4ccccc45)cc3)c3ccc4sc5ccccc5c4c3-c3ccc4oc5ccccc5c4c3)cc21 ; CC1(C)c2ccccc2-c2ccc(N(c3cccc(-c4ccc5c(c4)oc4ccccc45)c3)c3ccc4sc5ccccc5c4c3-c3ccc4oc5ccccc5c4c3)cc21]

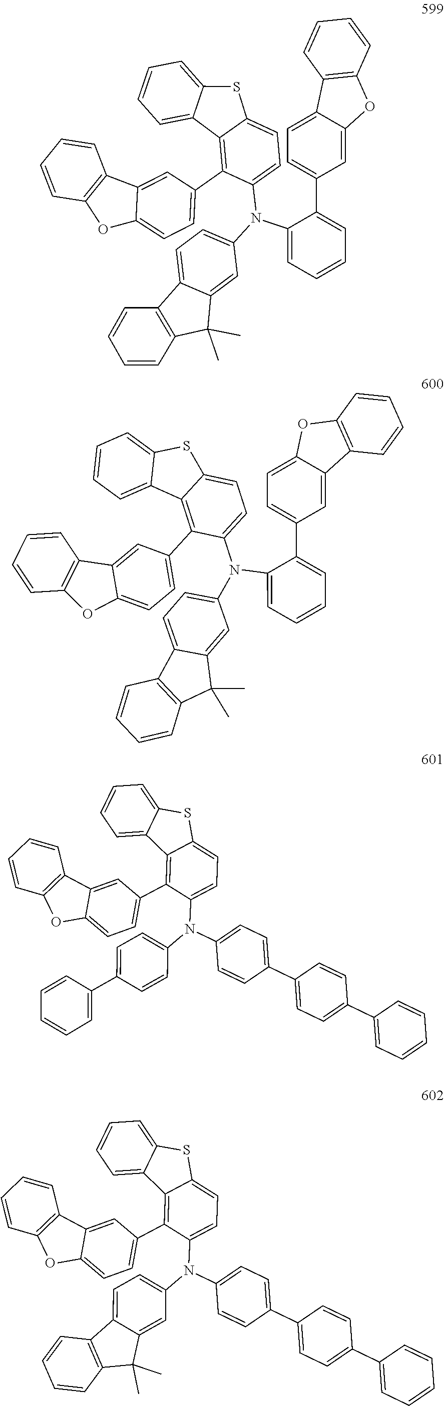

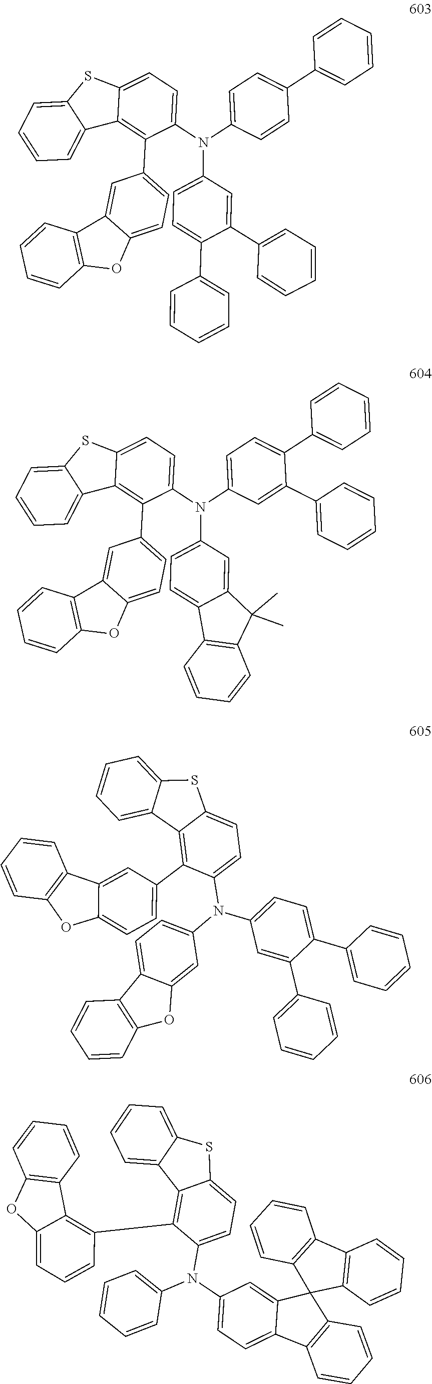 [SMILES: c1ccc(-c2ccc(N(c3ccc(-c4ccccc4)c(-c4ccccc4)c3)c3ccc4sc5ccccc5c4c3-c3ccc4oc5ccccc5c4c3)cc2)cc1 ; CC1(C)c2ccccc2-c2ccc(N(c3ccc(-c4ccccc4)c(-c4ccccc4)c3)c3ccc4sc5ccccc5c4c3-c3ccc4oc5ccccc5c4c3)cc21 ; c1ccc(-c2ccc(N(c3ccc4c(c3)oc3ccccc34)c3ccc4sc5ccccc5c4c3-c3ccc4oc5ccccc5c4c3)cc2-c2ccccc2)cc1 ; c1ccc(N(c2ccc3c(c2)C2(c4ccccc4-c4ccccc42)c2ccccc2-3)c2ccc3sc4ccccc4c3c2-c2cccc3oc4ccccc4c23)cc1]

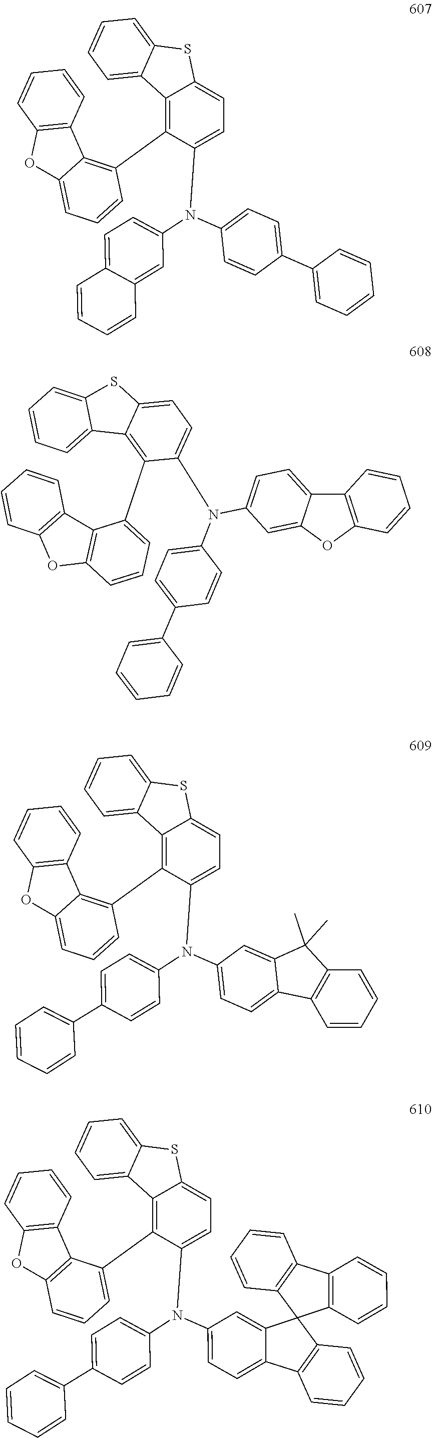 [SMILES: c1ccc(-c2ccc(N(c3ccc4ccccc4c3)c3ccc4sc5ccccc5c4c3-c3cccc4oc5ccccc5c34)cc2)cc1 ; c1ccc(-c2ccc(N(c3ccc4c(c3)oc3ccccc34)c3ccc4sc5ccccc5c4c3-c3cccc4oc5ccccc5c34)cc2)cc1 ; CC1(C)c2ccccc2-c2ccc(N(c3ccc(-c4ccccc4)cc3)c3ccc4sc5ccccc5c4c3-c3cccc4oc5ccccc5c34)cc21 ; c1ccc(-c2ccc(N(c3ccc4c(c3)C3(c5ccccc5-c5ccccc53)c3ccccc3-4)c3ccc4sc5ccccc5c4c3-c3cccc4oc5ccccc5c34)cc2)cc1]

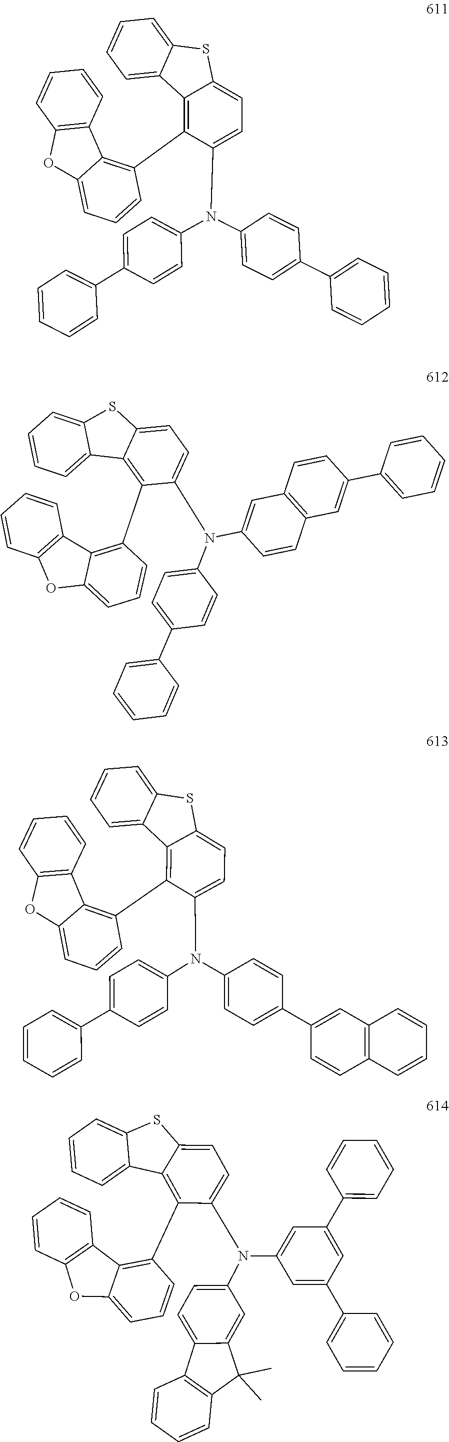 [SMILES: c1ccc(-c2ccc(N(c3ccc(-c4ccccc4)cc3)c3ccc4sc5ccccc5c4c3-c3cccc4oc5ccccc5c34)cc2)cc1 ; c1ccc(-c2ccc(N(c3ccc4cc(-c5ccccc5)ccc4c3)c3ccc4sc5ccccc5c4c3-c3cccc4oc5ccccc5c34)cc2)cc1 ; c1ccc(-c2ccc(N(c3ccc(-c4ccc5ccccc5c4)cc3)c3ccc4sc5ccccc5c4c3-c3cccc4oc5ccccc5c34)cc2)cc1 ; CC1(C)c2ccccc2-c2ccc(N(c3cc(-c4ccccc4)cc(-c4ccccc4)c3)c3ccc4sc5ccccc5c4c3-c3cccc4oc5ccccc5c34)cc21]

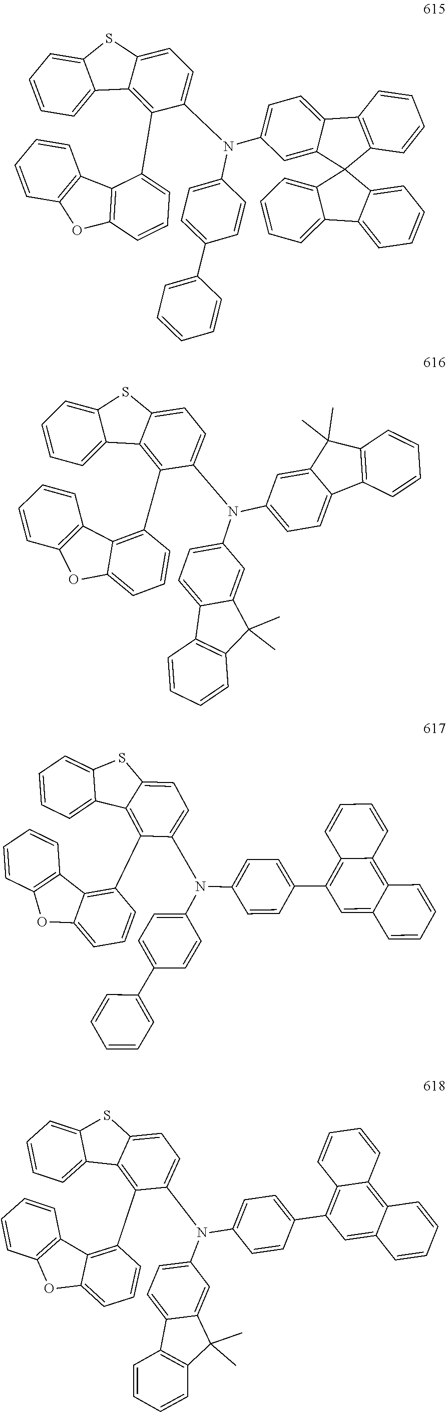 [SMILES: c1ccc(-c2ccc(N(c3ccc4c(c3)C3(c5ccccc5-c5ccccc53)c3ccccc3-4)c3ccc4sc5ccccc5c4c3-c3cccc4oc5ccccc5c34)cc2)cc1 ; CC1(C)c2ccccc2-c2ccc(N(c3ccc4c(c3)C(C)(C)c3ccccc3-4)c3ccc4sc5ccccc5c4c3-c3cccc4oc5ccccc5c34)cc21 ; c1ccc(-c2ccc(N(c3ccc(-c4cc5ccccc5c5ccccc45)cc3)c3ccc4sc5ccccc5c4c3-c3cccc4oc5ccccc5c34)cc2)cc1 ; CC1(C)c2ccccc2-c2ccc(N(c3ccc(-c4cc5ccccc5c5ccccc45)cc3)c3ccc4sc5ccccc5c4c3-c3cccc4oc5ccccc5c34)cc21]

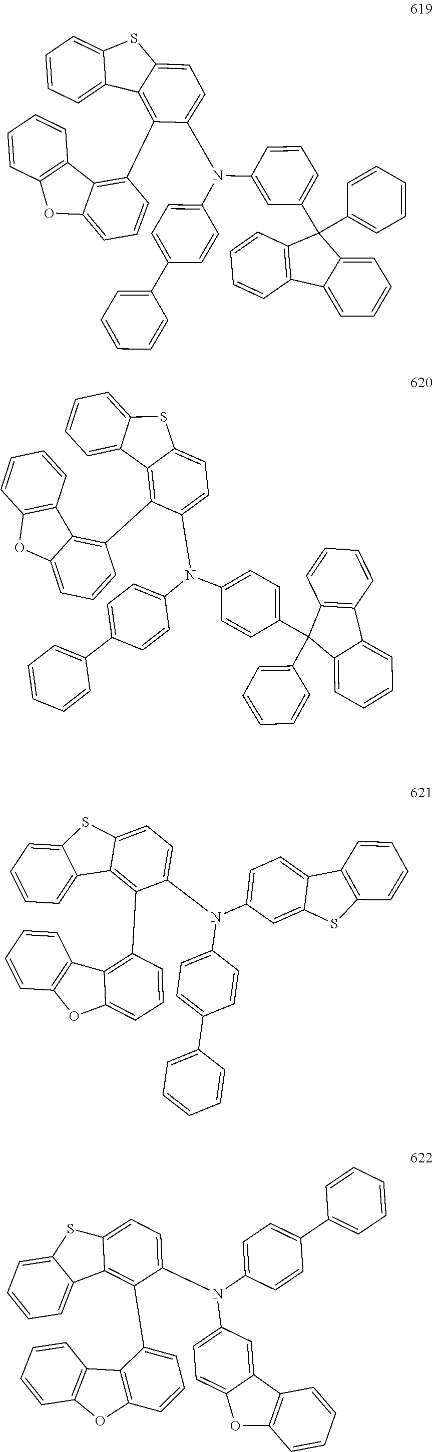

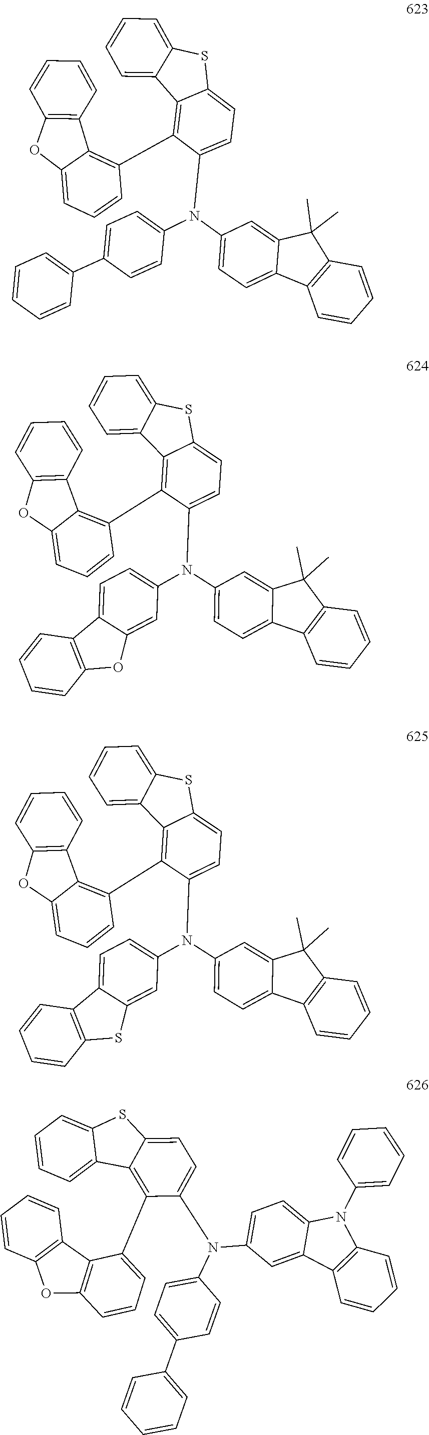 [SMILES: CC1(C)c2ccccc2-c2ccc(N(c3ccc(-c4ccccc4)cc3)c3ccc4sc5ccccc5c4c3-c3cccc4oc5ccccc5c34)cc21 ; CC1(C)c2ccccc2-c2ccc(N(c3ccc4c(c3)oc3ccccc34)c3ccc4sc5ccccc5c4c3-c3cccc4oc5ccccc5c34)cc21 ; CC1(C)c2ccccc2-c2ccc(N(c3ccc4c(c3)sc3ccccc34)c3ccc4sc5ccccc5c4c3-c3cccc4oc5ccccc5c34)cc21 ; c1ccc(-c2ccc(N(c3ccc4c(c3)c3ccccc3n4-c3ccccc3)c3ccc4sc5ccccc5c4c3-c3cccc4oc5ccccc5c34)cc2)cc1]

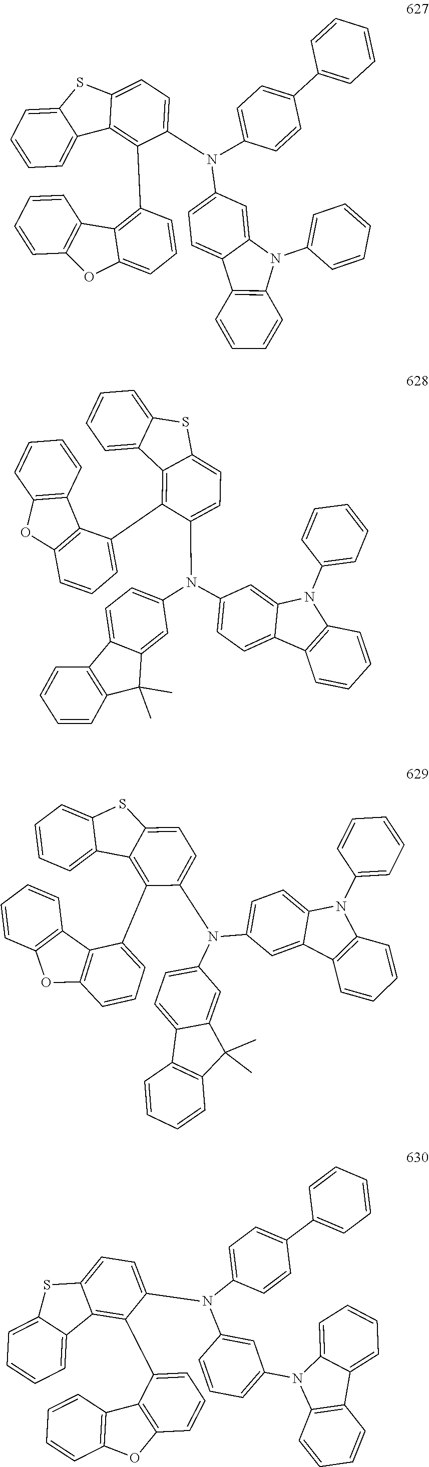 [SMILES: c1ccc(-c2ccc(N(c3ccc4c5ccccc5n(-c5ccccc5)c4c3)c3ccc4sc5ccccc5c4c3-c3cccc4oc5ccccc5c34)cc2)cc1 ; CC1(C)c2ccccc2-c2ccc(N(c3ccc4c5ccccc5n(-c5ccccc5)c4c3)c3ccc4sc5ccccc5c4c3-c3cccc4oc5ccccc5c34)cc21 ; CC1(C)c2ccccc2-c2ccc(N(c3ccc4c(c3)c3ccccc3n4-c3ccccc3)c3ccc4sc5ccccc5c4c3-c3cccc4oc5ccccc5c34)cc21 ; c1ccc(-c2ccc(N(c3cccc(-n4c5ccccc5c5ccccc54)c3)c3ccc4sc5ccccc5c4c3-c3cccc4oc5ccccc5c34)cc2)cc1]

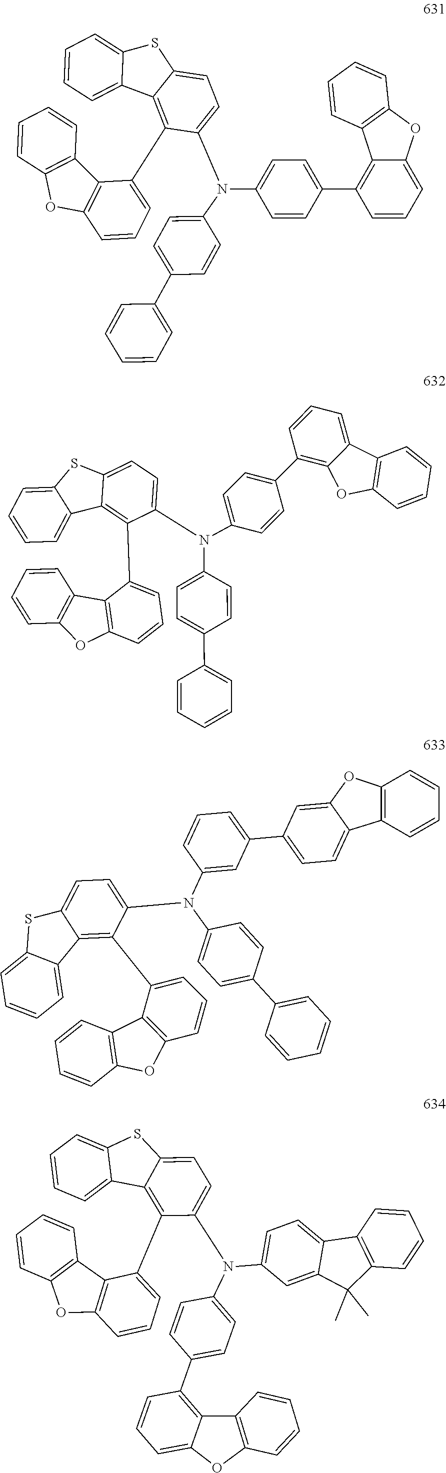 [SMILES: c1ccc(-c2ccc(N(c3ccc(-c4cccc5oc6ccccc6c45)cc3)c3ccc4sc5ccccc5c4c3-c3cccc4oc5ccccc5c34)cc2)cc1 ; c1ccc(-c2ccc(N(c3ccc(-c4cccc5c4oc4ccccc45)cc3)c3ccc4sc5ccccc5c4c3-c3cccc4oc5ccccc5c34)cc2)cc1 ; c1ccc(-c2ccc(N(c3cccc(-c4ccc5c(c4)oc4ccccc45)c3)c3ccc4sc5ccccc5c4c3-c3cccc4oc5ccccc5c34)cc2)cc1 ; CC1(C)c2ccccc2-c2ccc(N(c3ccc(-c4cccc5oc6ccccc6c45)cc3)c3ccc4sc5ccccc5c4c3-c3cccc4oc5ccccc5c34)cc21]

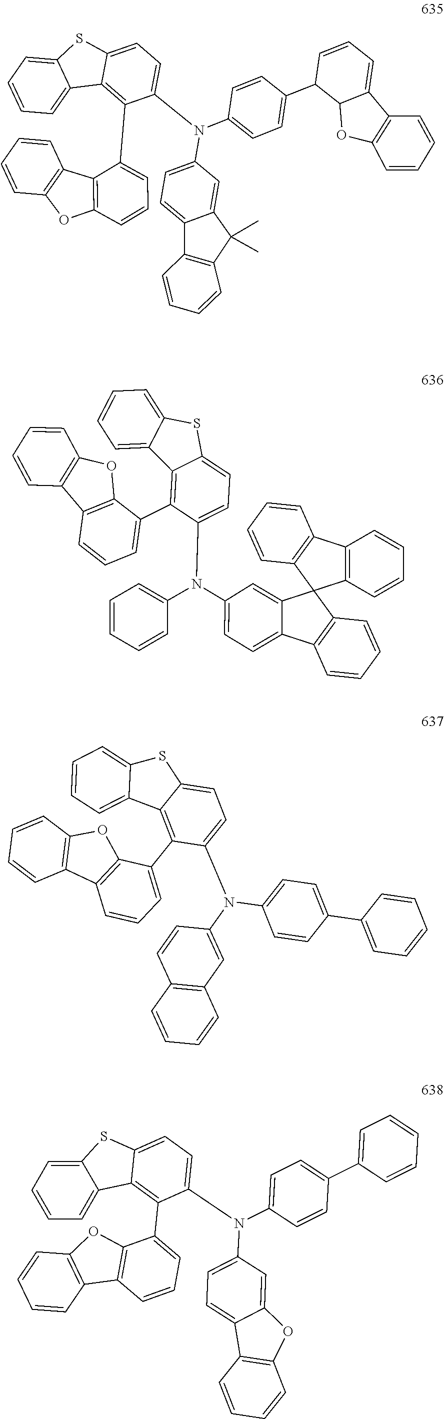 [SMILES: CC1(C)c2ccccc2-c2ccc(N(c3ccc(C4C=CC=C5c6ccccc6OC54)cc3)c3ccc4sc5ccccc5c4c3-c3cccc4oc5ccccc5c34)cc21 ; c1ccc(N(c2ccc3c(c2)C2(c4ccccc4-c4ccccc42)c2ccccc2-3)c2ccc3sc4ccccc4c3c2-c2cccc3c2oc2ccccc23)cc1 ; c1ccc(-c2ccc(N(c3ccc4ccccc4c3)c3ccc4sc5ccccc5c4c3-c3cccc4c3oc3ccccc34)cc2)cc1 ; c1ccc(-c2ccc(N(c3ccc4c(c3)oc3ccccc34)c3ccc4sc5ccccc5c4c3-c3cccc4c3oc3ccccc34)cc2)cc1]

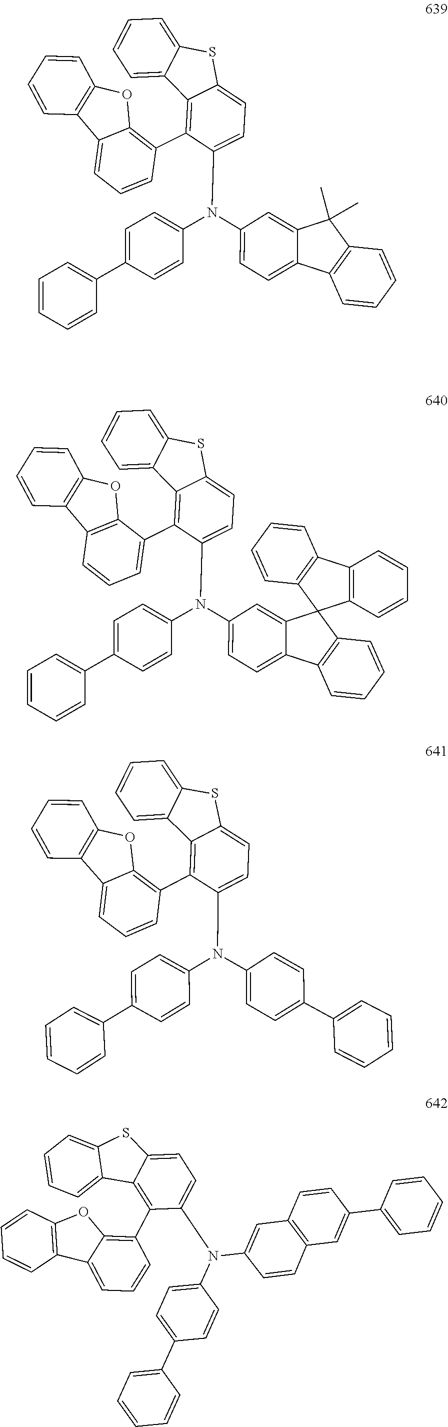 [SMILES: CC1(C)c2ccccc2-c2ccc(N(c3ccc(-c4ccccc4)cc3)c3ccc4sc5ccccc5c4c3-c3cccc4c3oc3ccccc34)cc21 ; c1ccc(-c2ccc(N(c3ccc4c(c3)C3(c5ccccc5-c5ccccc53)c3ccccc3-4)c3ccc4sc5ccccc5c4c3-c3cccc4c3oc3ccccc34)cc2)cc1 ; c1ccc(-c2ccc(N(c3ccc(-c4ccccc4)cc3)c3ccc4sc5ccccc5c4c3-c3cccc4c3oc3ccccc34)cc2)cc1 ; c1ccc(-c2ccc(N(c3ccc4cc(-c5ccccc5)ccc4c3)c3ccc4sc5ccccc5c4c3-c3cccc4c3oc3ccccc34)cc2)cc1]

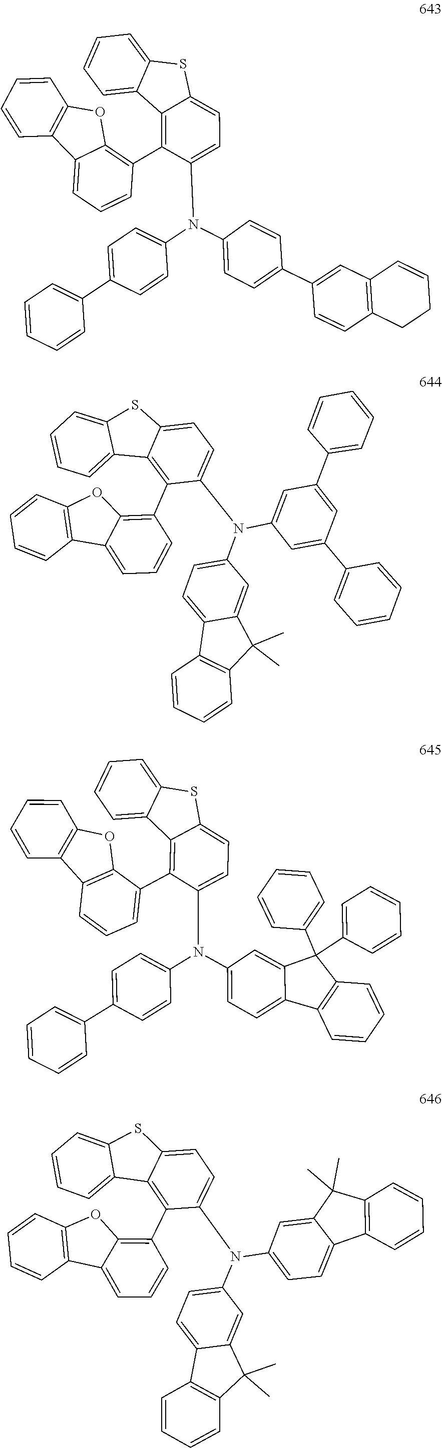 [SMILES: C1=Cc2cc(-c3ccc(N(c4ccc(-c5ccccc5)cc4)c4ccc5sc6ccccc6c5c4-c4cccc5c4oc4ccccc45)cc3)ccc2CC1 ; CC1(C)c2ccccc2-c2ccc(N(c3cc(-c4ccccc4)cc(-c4ccccc4)c3)c3ccc4sc5ccccc5c4c3-c3cccc4c3oc3ccccc34)cc21 ; c1ccc(-c2ccc(N(c3ccc4c(c3)C(c3ccccc3)(c3ccccc3)c3ccccc3-4)c3ccc4sc5ccccc5c4c3-c3cccc4c3oc3ccccc34)cc2)cc1 ; CC1(C)c2ccccc2-c2ccc(N(c3ccc4c(c3)C(C)(C)c3ccccc3-4)c3ccc4sc5ccccc5c4c3-c3cccc4c3oc3ccccc34)cc21]

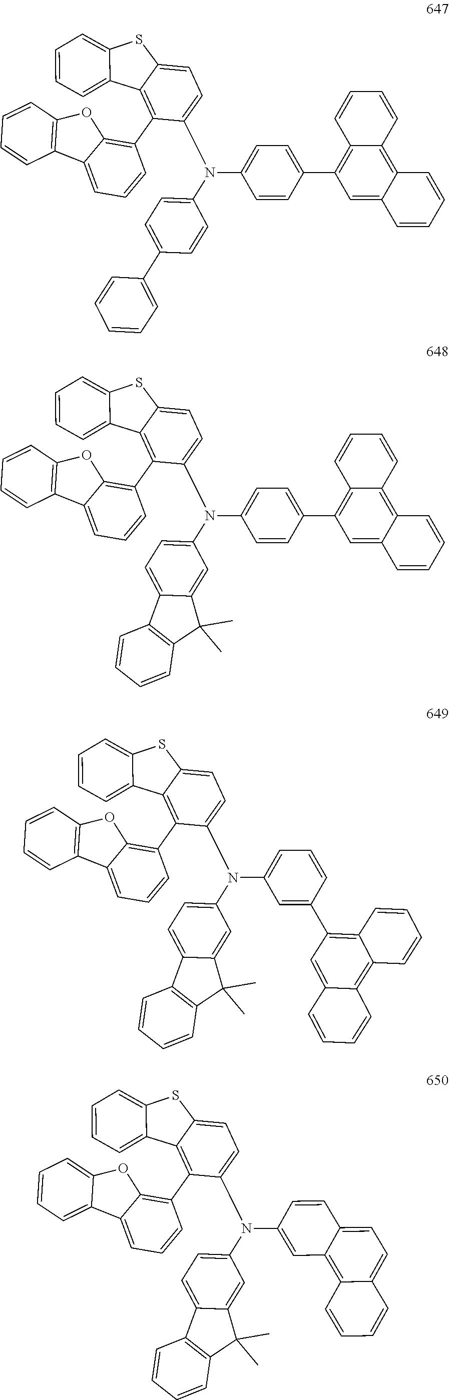 [SMILES: c1ccc(-c2ccc(N(c3ccc(-c4cc5ccccc5c5ccccc45)cc3)c3ccc4sc5ccccc5c4c3-c3cccc4c3oc3ccccc34)cc2)cc1 ; CC1(C)c2ccccc2-c2ccc(N(c3ccc(-c4cc5ccccc5c5ccccc45)cc3)c3ccc4sc5ccccc5c4c3-c3cccc4c3oc3ccccc34)cc21 ; CC1(C)c2ccccc2-c2ccc(N(c3cccc(-c4cc5ccccc5c5ccccc45)c3)c3ccc4sc5ccccc5c4c3-c3cccc4c3oc3ccccc34)cc21 ; CC1(C)c2ccccc2-c2ccc(N(c3ccc4ccc5ccccc5c4c3)c3ccc4sc5ccccc5c4c3-c3cccc4c3oc3ccccc34)cc21]

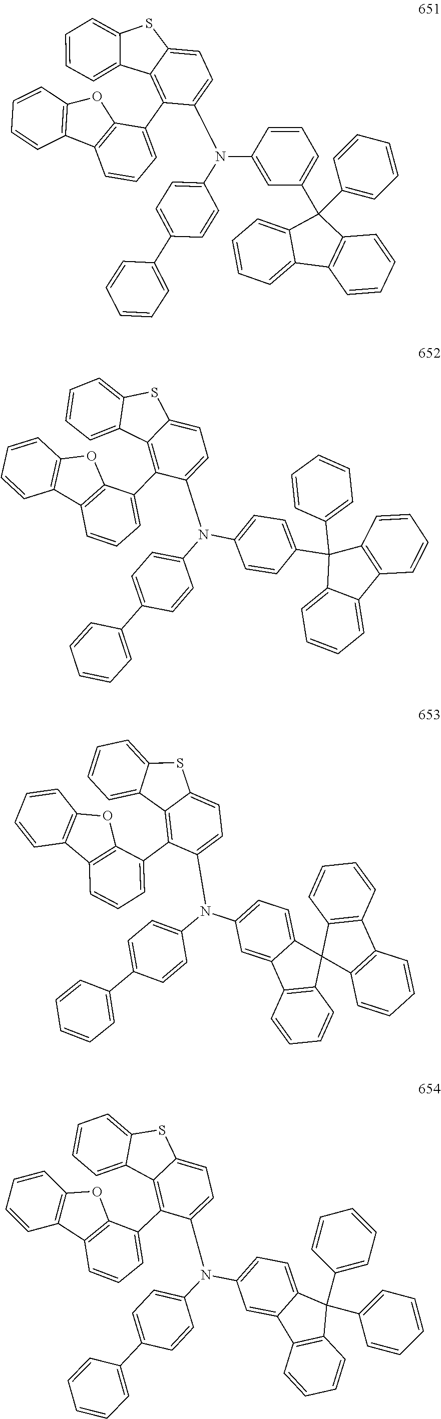 [SMILES: c1ccc(-c2ccc(N(c3cccc(C4(c5ccccc5)c5ccccc5-c5ccccc54)c3)c3ccc4sc5ccccc5c4c3-c3cccc4c3oc3ccccc34)cc2)cc1 ; c1ccc(-c2ccc(N(c3ccc(C4(c5ccccc5)c5ccccc5-c5ccccc54)cc3)c3ccc4sc5ccccc5c4c3-c3cccc4c3oc3ccccc34)cc2)cc1 ; c1ccc(-c2ccc(N(c3ccc4c(c3)-c3ccccc3C43c4ccccc4-c4ccccc43)c3ccc4sc5ccccc5c4c3-c3cccc4c3oc3ccccc34)cc2)cc1 ; c1ccc(-c2ccc(N(c3ccc4c(c3)-c3ccccc3C4(c3ccccc3)c3ccccc3)c3ccc4sc5ccccc5c4c3-c3cccc4c3oc3ccccc34)cc2)cc1]

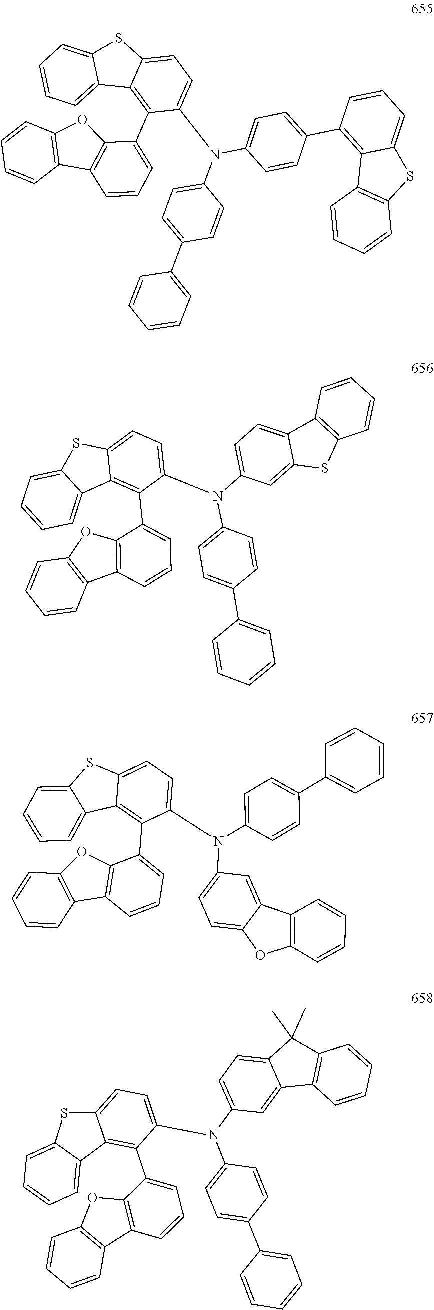 [SMILES: c1ccc(-c2ccc(N(c3ccc(-c4cccc5sc6ccccc6c45)cc3)c3ccc4sc5ccccc5c4c3-c3cccc4c3oc3ccccc34)cc2)cc1 ; c1ccc(-c2ccc(N(c3ccc4c(c3)sc3ccccc34)c3ccc4sc5ccccc5c4c3-c3cccc4c3oc3ccccc34)cc2)cc1 ; c1ccc(-c2ccc(N(c3ccc4oc5ccccc5c4c3)c3ccc4sc5ccccc5c4c3-c3cccc4c3oc3ccccc34)cc2)cc1 ; CC1(C)c2ccccc2-c2cc(N(c3ccc(-c4ccccc4)cc3)c3ccc4sc5ccccc5c4c3-c3cccc4c3oc3ccccc34)ccc21]

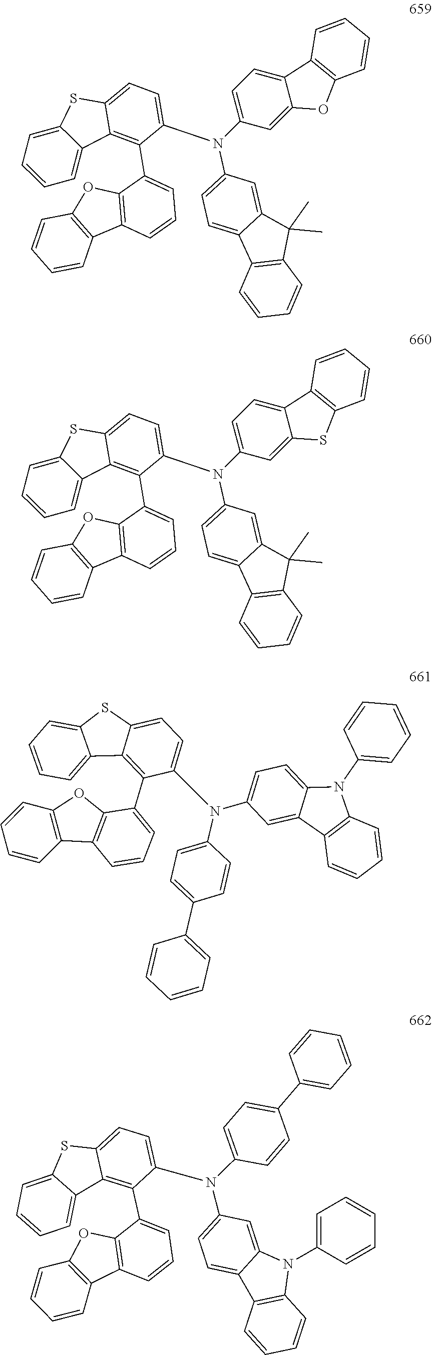 [SMILES: CC1(C)c2ccccc2-c2ccc(N(c3ccc4c(c3)oc3ccccc34)c3ccc4sc5ccccc5c4c3-c3cccc4c3oc3ccccc34)cc21 ; CC1(C)c2ccccc2-c2ccc(N(c3ccc4c(c3)sc3ccccc34)c3ccc4sc5ccccc5c4c3-c3cccc4c3oc3ccccc34)cc21 ; c1ccc(-c2ccc(N(c3ccc4c(c3)c3ccccc3n4-c3ccccc3)c3ccc4sc5ccccc5c4c3-c3cccc4c3oc3ccccc34)cc2)cc1 ; c1ccc(-c2ccc(N(c3ccc4c5ccccc5n(-c5ccccc5)c4c3)c3ccc4sc5ccccc5c4c3-c3cccc4c3oc3ccccc34)cc2)cc1]

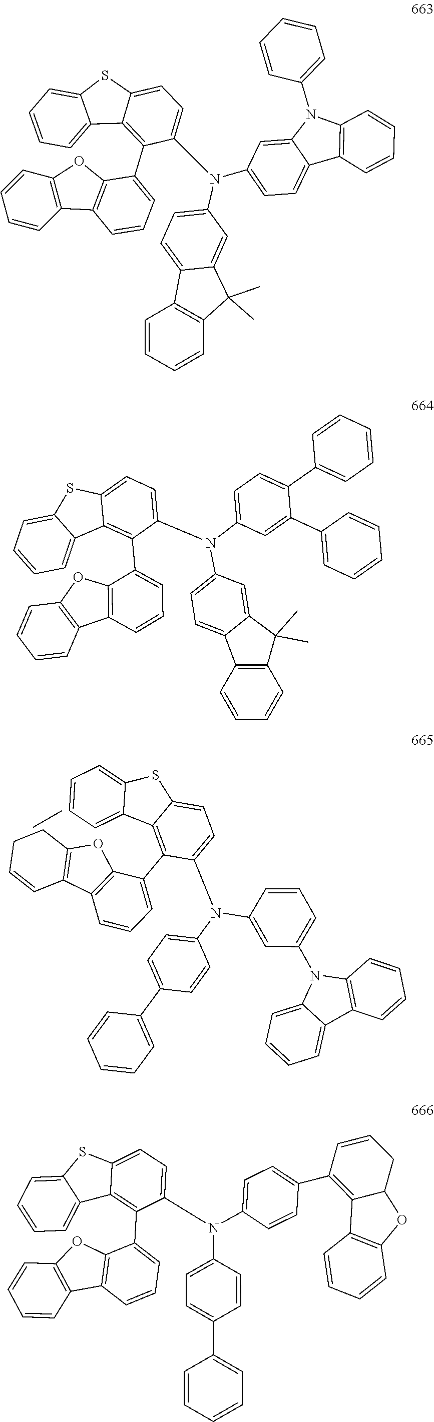

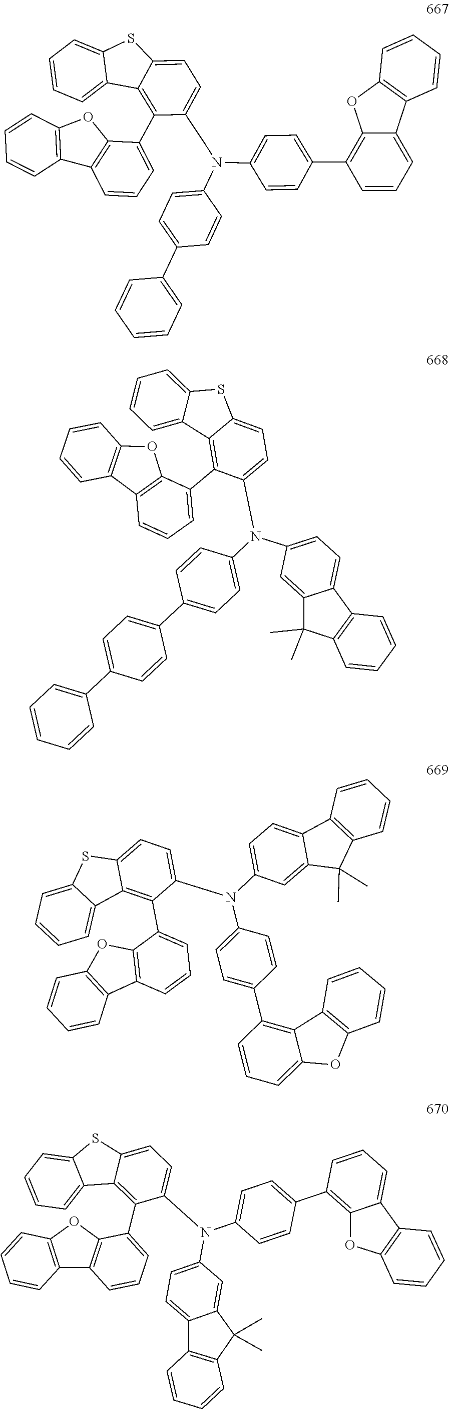

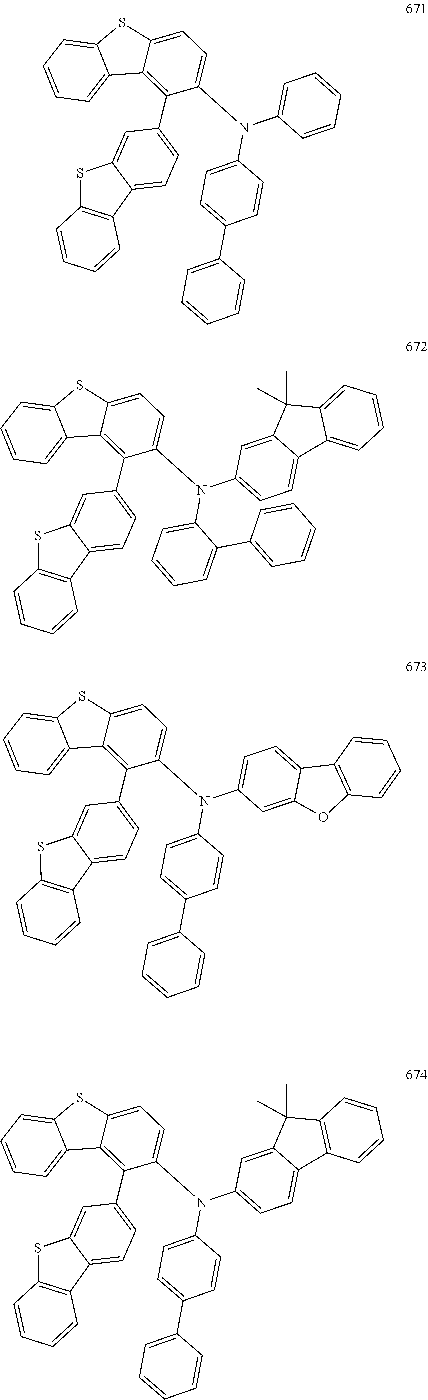 [SMILES: c1ccc(-c2ccc(N(c3ccccc3)c3ccc4sc5ccccc5c4c3-c3ccc4c(c3)sc3ccccc34)cc2)cc1 ; CC1(C)c2ccccc2-c2ccc(N(c3ccccc3-c3ccccc3)c3ccc4sc5ccccc5c4c3-c3ccc4c(c3)sc3ccccc34)cc21 ; c1ccc(-c2ccc(N(c3ccc4c(c3)oc3ccccc34)c3ccc4sc5ccccc5c4c3-c3ccc4c(c3)sc3ccccc34)cc2)cc1 ; CC1(C)c2ccccc2-c2ccc(N(c3ccc(-c4ccccc4)cc3)c3ccc4sc5ccccc5c4c3-c3ccc4c(c3)sc3ccccc34)cc21]

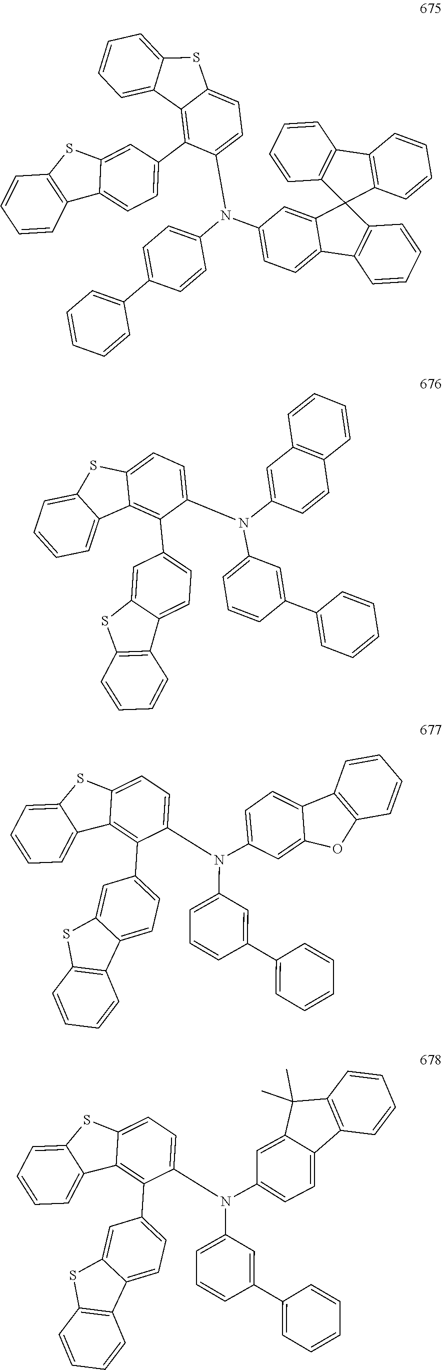 [SMILES: c1ccc(-c2ccc(N(c3ccc4c(c3)C3(c5ccccc5-c5ccccc53)c3ccccc3-4)c3ccc4sc5ccccc5c4c3-c3ccc4c(c3)sc3ccccc34)cc2)cc1 ; c1ccc(-c2cccc(N(c3ccc4ccccc4c3)c3ccc4sc5ccccc5c4c3-c3ccc4c(c3)sc3ccccc34)c2)cc1 ; c1ccc(-c2cccc(N(c3ccc4c(c3)oc3ccccc34)c3ccc4sc5ccccc5c4c3-c3ccc4c(c3)sc3ccccc34)c2)cc1 ; CC1(C)c2ccccc2-c2ccc(N(c3cccc(-c4ccccc4)c3)c3ccc4sc5ccccc5c4c3-c3ccc4c(c3)sc3ccccc34)cc21]

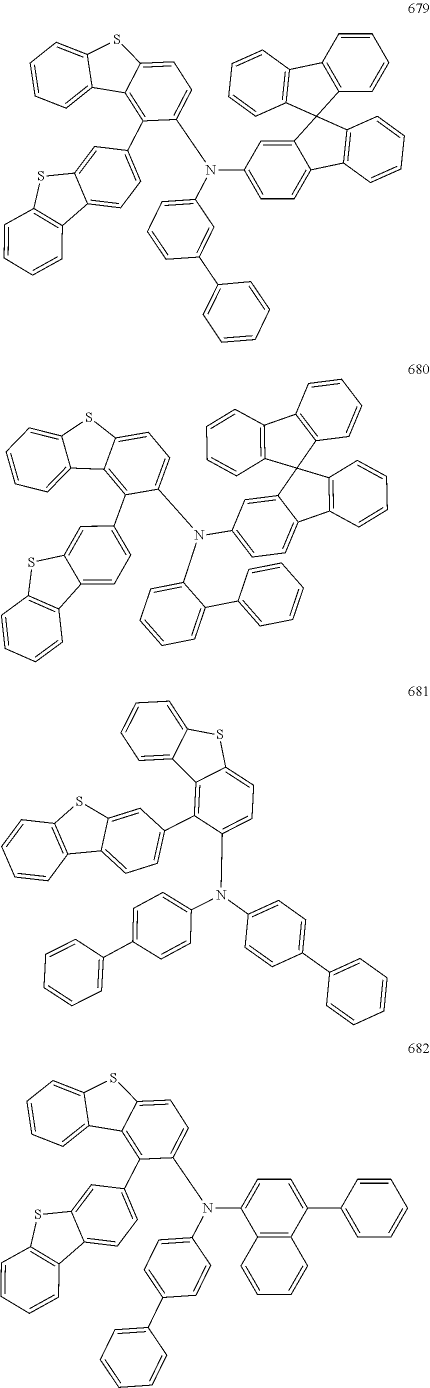 [SMILES: c1ccc(-c2cccc(N(c3ccc4c(c3)C3(c5ccccc5-c5ccccc53)c3ccccc3-4)c3ccc4sc5ccccc5c4c3-c3ccc4c(c3)sc3ccccc34)c2)cc1 ; c1ccc(-c2ccccc2N(c2ccc3c(c2)C2(c4ccccc4-c4ccccc42)c2ccccc2-3)c2ccc3sc4ccccc4c3c2-c2ccc3c(c2)sc2ccccc23)cc1 ; c1ccc(-c2ccc(N(c3ccc(-c4ccccc4)cc3)c3ccc4sc5ccccc5c4c3-c3ccc4c(c3)sc3ccccc34)cc2)cc1 ; c1ccc(-c2ccc(N(c3ccc4sc5ccccc5c4c3-c3ccc4c(c3)sc3ccccc34)c3ccc(-c4ccccc4)c4ccccc34)cc2)cc1]

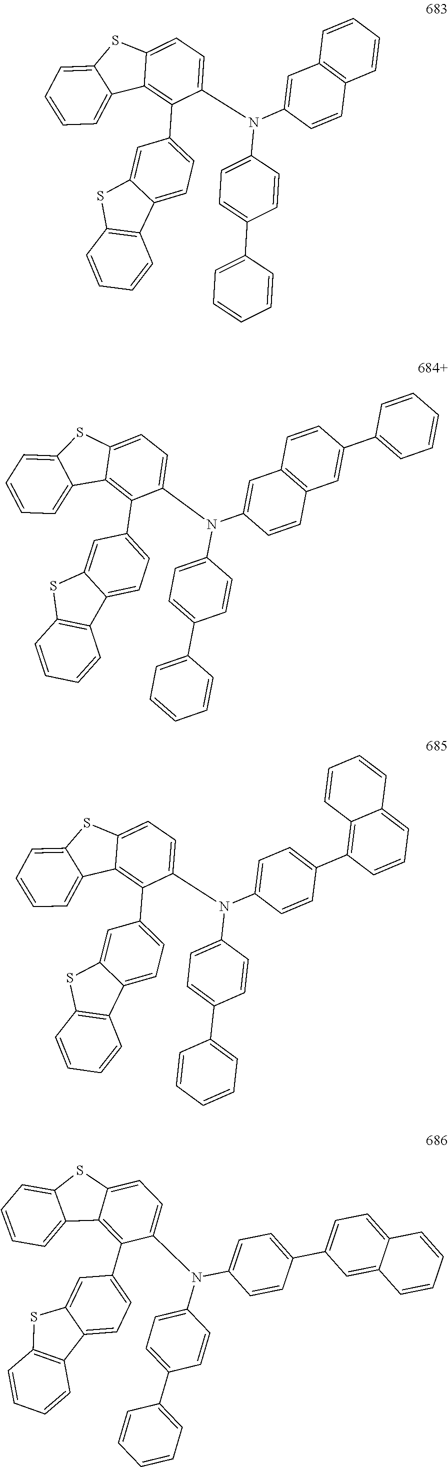 [SMILES: c1ccc(-c2ccc(N(c3ccc4ccccc4c3)c3ccc4sc5ccccc5c4c3-c3ccc4c(c3)sc3ccccc34)cc2)cc1 ; c1ccc(-c2ccc(N(c3ccc4cc(-c5ccccc5)ccc4c3)c3ccc4sc5ccccc5c4c3-c3ccc4c(c3)sc3ccccc34)cc2)cc1 ; c1ccc(-c2ccc(N(c3ccc(-c4cccc5ccccc45)cc3)c3ccc4sc5ccccc5c4c3-c3ccc4c(c3)sc3ccccc34)cc2)cc1 ; c1ccc(-c2ccc(N(c3ccc(-c4ccc5ccccc5c4)cc3)c3ccc4sc5ccccc5c4c3-c3ccc4c(c3)sc3ccccc34)cc2)cc1]

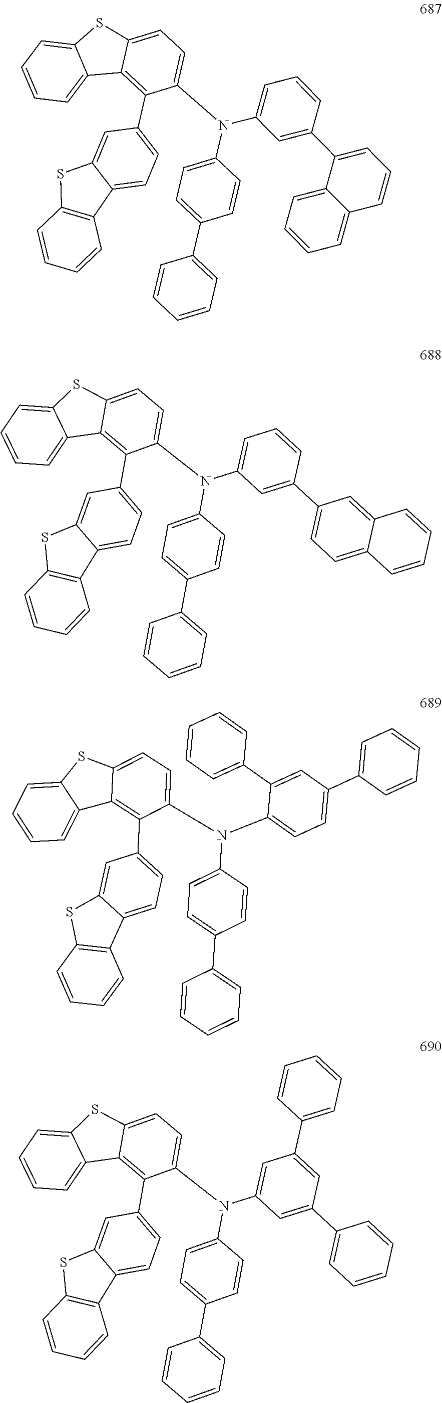 [SMILES: c1ccc(-c2ccc(N(c3cccc(-c4cccc5ccccc45)c3)c3ccc4sc5ccccc5c4c3-c3ccc4c(c3)sc3ccccc34)cc2)cc1 ; c1ccc(-c2ccc(N(c3cccc(-c4ccc5ccccc5c4)c3)c3ccc4sc5ccccc5c4c3-c3ccc4c(c3)sc3ccccc34)cc2)cc1 ; c1ccc(-c2ccc(N(c3ccc(-c4ccccc4)cc3-c3ccccc3)c3ccc4sc5ccccc5c4c3-c3ccc4c(c3)sc3ccccc34)cc2)cc1 ; c1ccc(-c2ccc(N(c3cc(-c4ccccc4)cc(-c4ccccc4)c3)c3ccc4sc5ccccc5c4c3-c3ccc4c(c3)sc3ccccc34)cc2)cc1]

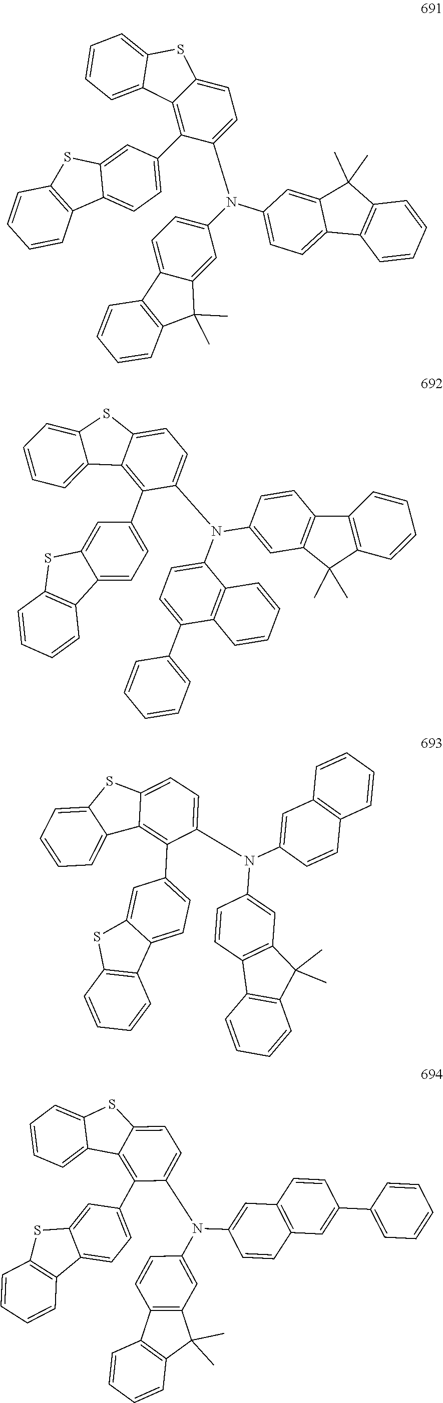 [SMILES: CC1(C)c2ccccc2-c2ccc(N(c3ccc4c(c3)C(C)(C)c3ccccc3-4)c3ccc4sc5ccccc5c4c3-c3ccc4c(c3)sc3ccccc34)cc21 ; CC1(C)c2ccccc2-c2ccc(N(c3ccc4sc5ccccc5c4c3-c3ccc4c(c3)sc3ccccc34)c3ccc(-c4ccccc4)c4ccccc34)cc21 ; CC1(C)c2ccccc2-c2ccc(N(c3ccc4ccccc4c3)c3ccc4sc5ccccc5c4c3-c3ccc4c(c3)sc3ccccc34)cc21 ; CC1(C)c2ccccc2-c2ccc(N(c3ccc4cc(-c5ccccc5)ccc4c3)c3ccc4sc5ccccc5c4c3-c3ccc4c(c3)sc3ccccc34)cc21]

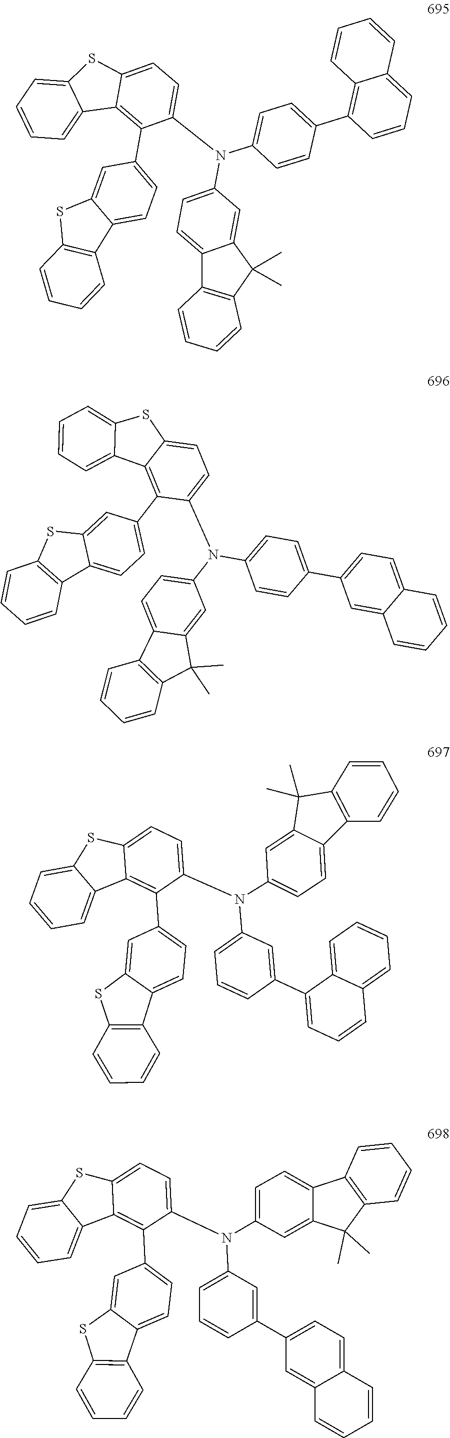 [SMILES: CC1(C)c2ccccc2-c2ccc(N(c3ccc(-c4cccc5ccccc45)cc3)c3ccc4sc5ccccc5c4c3-c3ccc4c(c3)sc3ccccc34)cc21 ; CC1(C)c2ccccc2-c2ccc(N(c3ccc(-c4ccc5ccccc5c4)cc3)c3ccc4sc5ccccc5c4c3-c3ccc4c(c3)sc3ccccc34)cc21 ; CC1(C)c2ccccc2-c2ccc(N(c3cccc(-c4cccc5ccccc45)c3)c3ccc4sc5ccccc5c4c3-c3ccc4c(c3)sc3ccccc34)cc21 ; CC1(C)c2ccccc2-c2ccc(N(c3cccc(-c4ccc5ccccc5c4)c3)c3ccc4sc5ccccc5c4c3-c3ccc4c(c3)sc3ccccc34)cc21]

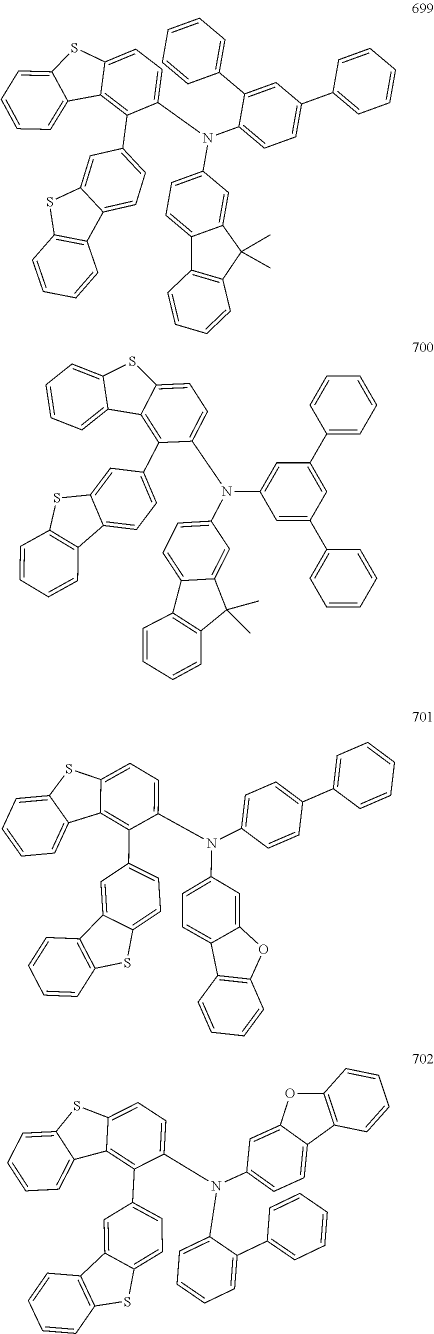 [SMILES: CC1(C)c2ccccc2-c2ccc(N(c3ccc(-c4ccccc4)cc3-c3ccccc3)c3ccc4sc5ccccc5c4c3-c3ccc4c(c3)sc3ccccc34)cc21 ; CC1(C)c2ccccc2-c2ccc(N(c3cc(-c4ccccc4)cc(-c4ccccc4)c3)c3ccc4sc5ccccc5c4c3-c3ccc4c(c3)sc3ccccc34)cc21 ; c1ccc(-c2ccc(N(c3ccc4c(c3)oc3ccccc34)c3ccc4sc5ccccc5c4c3-c3ccc4sc5ccccc5c4c3)cc2)cc1 ; c1ccc(-c2ccccc2N(c2ccc3c(c2)oc2ccccc23)c2ccc3sc4ccccc4c3c2-c2ccc3sc4ccccc4c3c2)cc1]

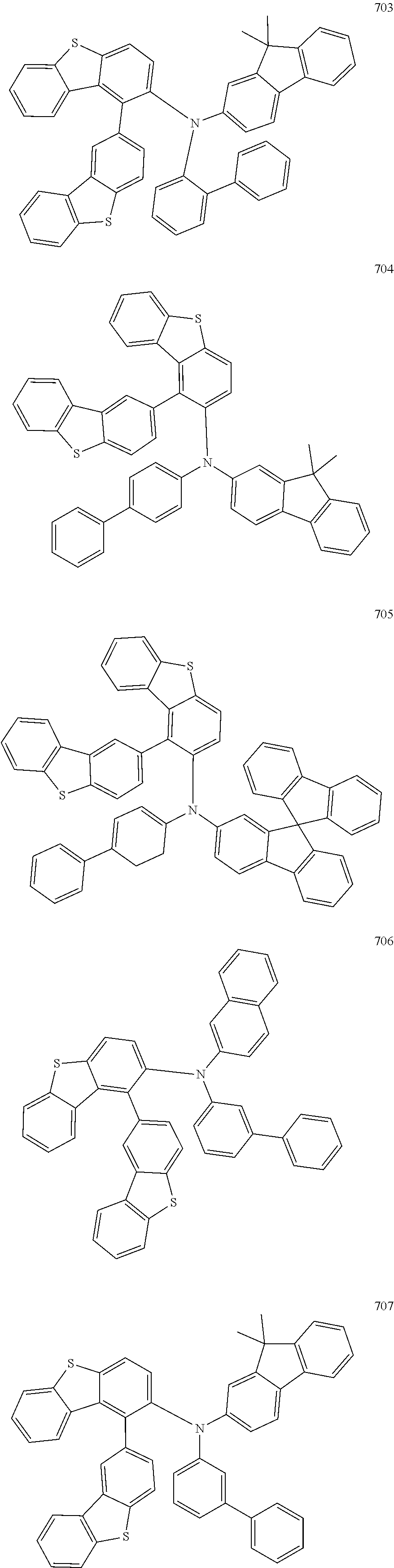 [SMILES: CC1(C)c2ccccc2-c2ccc(N(c3ccccc3-c3ccccc3)c3ccc4sc5ccccc5c4c3-c3ccc4sc5ccccc5c4c3)cc21 ; CC1(C)c2ccccc2-c2ccc(N(c3ccc(-c4ccccc4)cc3)c3ccc4sc5ccccc5c4c3-c3ccc4sc5ccccc5c4c3)cc21 ; C1=C(c2ccccc2)CCC(N(c2ccc3c(c2)C2(c4ccccc4-c4ccccc42)c2ccccc2-3)c2ccc3sc4ccccc4c3c2-c2ccc3sc4ccccc4c3c2)=C1 ; c1ccc(-c2cccc(N(c3ccc4ccccc4c3)c3ccc4sc5ccccc5c4c3-c3ccc4sc5ccccc5c4c3)c2)cc1 ; CC1(C)c2ccccc2-c2ccc(N(c3cccc(-c4ccccc4)c3)c3ccc4sc5ccccc5c4c3-c3ccc4sc5ccccc5c4c3)cc21]

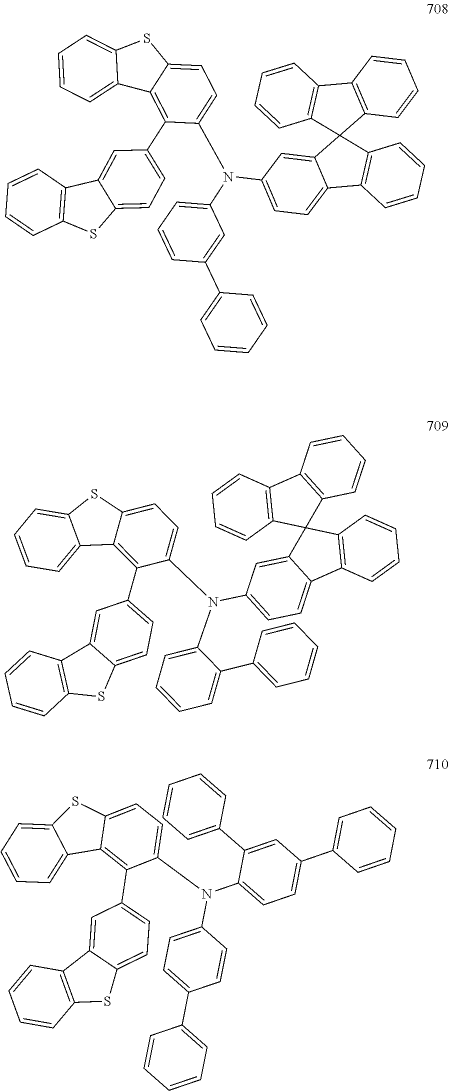 [SMILES: c1ccc(-c2cccc(N(c3ccc4c(c3)C3(c5ccccc5-c5ccccc53)c3ccccc3-4)c3ccc4sc5ccccc5c4c3-c3ccc4sc5ccccc5c4c3)c2)cc1 ; c1ccc(-c2ccccc2N(c2ccc3c(c2)C2(c4ccccc4-c4ccccc42)c2ccccc2-3)c2ccc3sc4ccccc4c3c2-c2ccc3sc4ccccc4c3c2)cc1 ; c1ccc(-c2ccc(N(c3ccc(-c4ccccc4)cc3-c3ccccc3)c3ccc4sc5ccccc5c4c3-c3ccc4sc5ccccc5c4c3)cc2)cc1]

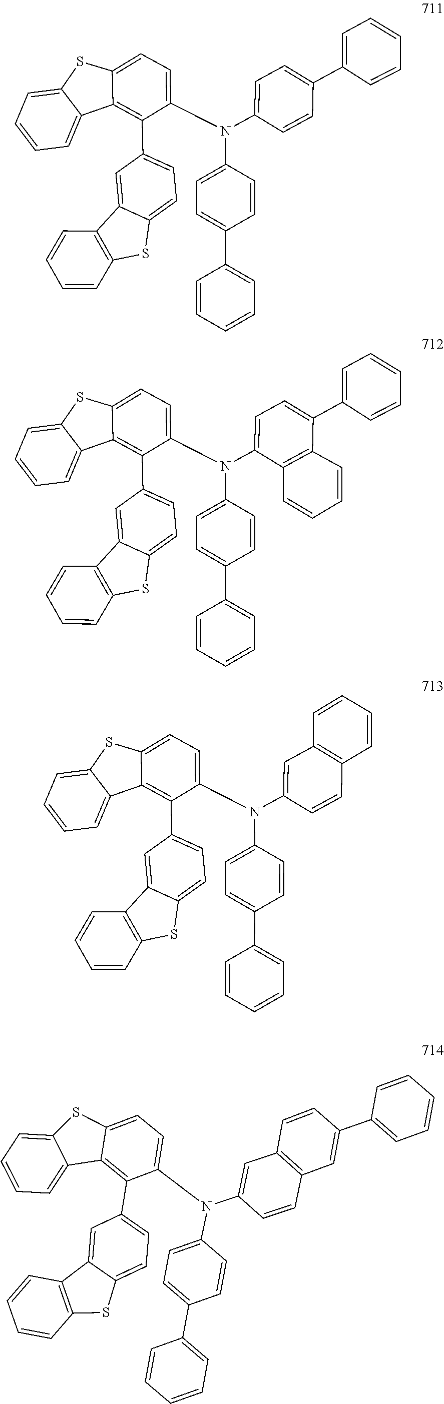 [SMILES: c1ccc(-c2ccc(N(c3ccc(-c4ccccc4)cc3)c3ccc4sc5ccccc5c4c3-c3ccc4sc5ccccc5c4c3)cc2)cc1 ; c1ccc(-c2ccc(N(c3ccc4sc5ccccc5c4c3-c3ccc4sc5ccccc5c4c3)c3ccc(-c4ccccc4)c4ccccc34)cc2)cc1 ; c1ccc(-c2ccc(N(c3ccc4ccccc4c3)c3ccc4sc5ccccc5c4c3-c3ccc4sc5ccccc5c4c3)cc2)cc1 ; c1ccc(-c2ccc(N(c3ccc4cc(-c5ccccc5)ccc4c3)c3ccc4sc5ccccc5c4c3-c3ccc4sc5ccccc5c4c3)cc2)cc1]

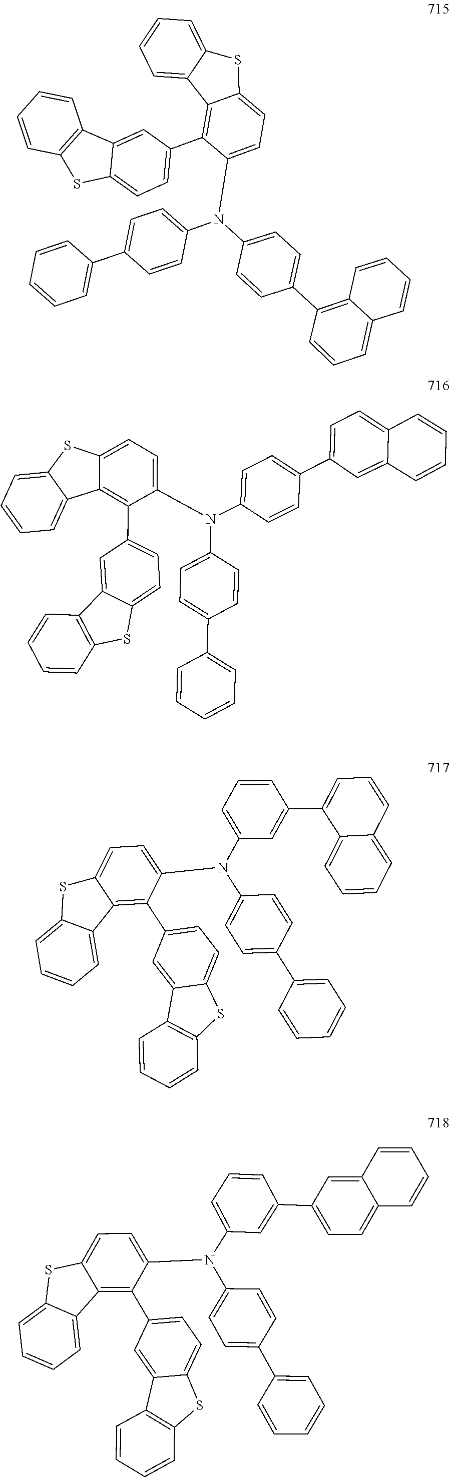 [SMILES: c1ccc(-c2ccc(N(c3ccc(-c4cccc5ccccc45)cc3)c3ccc4sc5ccccc5c4c3-c3ccc4sc5ccccc5c4c3)cc2)cc1 ; c1ccc(-c2ccc(N(c3ccc(-c4ccc5ccccc5c4)cc3)c3ccc4sc5ccccc5c4c3-c3ccc4sc5ccccc5c4c3)cc2)cc1 ; c1ccc(-c2ccc(N(c3cccc(-c4cccc5ccccc45)c3)c3ccc4sc5ccccc5c4c3-c3ccc4sc5ccccc5c4c3)cc2)cc1 ; c1ccc(-c2ccc(N(c3cccc(-c4ccc5ccccc5c4)c3)c3ccc4sc5ccccc5c4c3-c3ccc4sc5ccccc5c4c3)cc2)cc1]

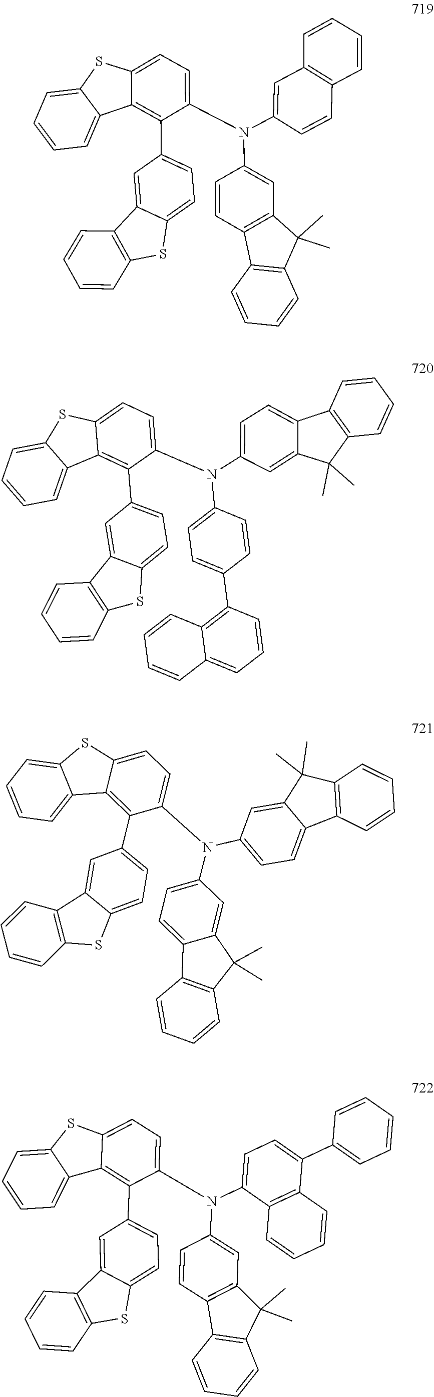 [SMILES: CC1(C)c2ccccc2-c2ccc(N(c3ccc4ccccc4c3)c3ccc4sc5ccccc5c4c3-c3ccc4sc5ccccc5c4c3)cc21 ; CC1(C)c2ccccc2-c2ccc(N(c3ccc(-c4cccc5ccccc45)cc3)c3ccc4sc5ccccc5c4c3-c3ccc4sc5ccccc5c4c3)cc21 ; CC1(C)c2ccccc2-c2ccc(N(c3ccc4c(c3)C(C)(C)c3ccccc3-4)c3ccc4sc5ccccc5c4c3-c3ccc4sc5ccccc5c4c3)cc21 ; CC1(C)c2ccccc2-c2ccc(N(c3ccc4sc5ccccc5c4c3-c3ccc4sc5ccccc5c4c3)c3ccc(-c4ccccc4)c4ccccc34)cc21]

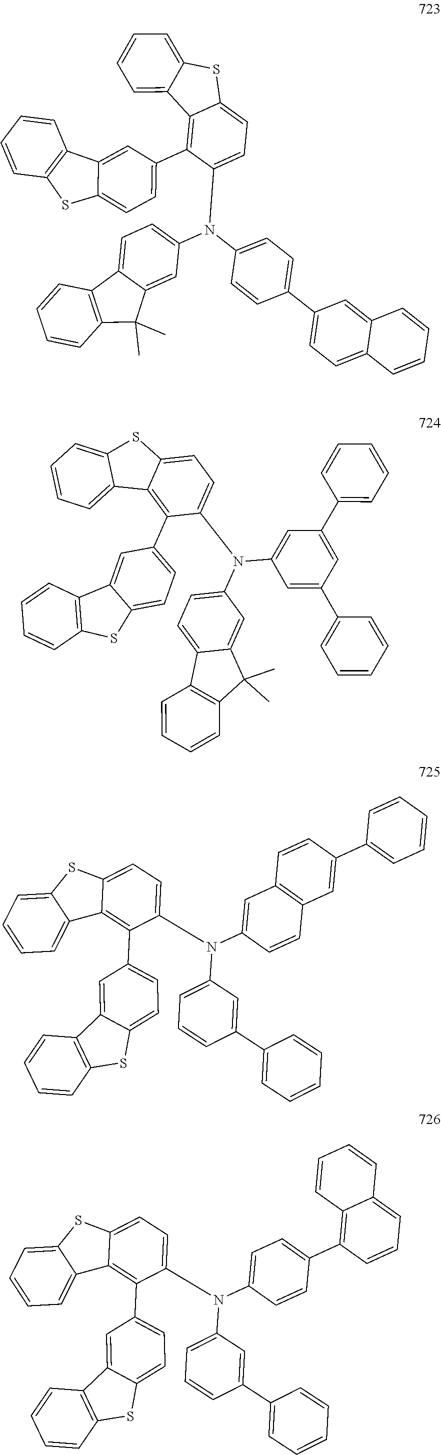 [SMILES: CC1(C)c2ccccc2-c2ccc(N(c3ccc(-c4ccc5ccccc5c4)cc3)c3ccc4sc5ccccc5c4c3-c3ccc4sc5ccccc5c4c3)cc21 ; CC1(C)c2ccccc2-c2ccc(N(c3cc(-c4ccccc4)cc(-c4ccccc4)c3)c3ccc4sc5ccccc5c4c3-c3ccc4sc5ccccc5c4c3)cc21 ; c1ccc(-c2cccc(N(c3ccc4cc(-c5ccccc5)ccc4c3)c3ccc4sc5ccccc5c4c3-c3ccc4sc5ccccc5c4c3)c2)cc1 ; c1ccc(-c2cccc(N(c3ccc(-c4cccc5ccccc45)cc3)c3ccc4sc5ccccc5c4c3-c3ccc4sc5ccccc5c4c3)c2)cc1]

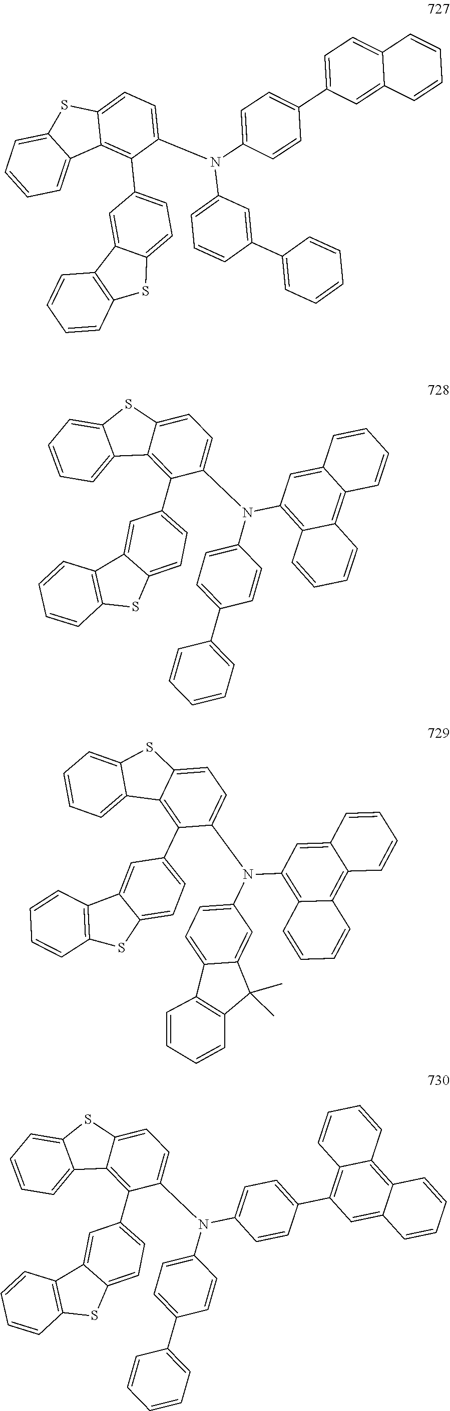 [SMILES: c1ccc(-c2cccc(N(c3ccc(-c4ccc5ccccc5c4)cc3)c3ccc4sc5ccccc5c4c3-c3ccc4sc5ccccc5c4c3)c2)cc1 ; c1ccc(-c2ccc(N(c3ccc4sc5ccccc5c4c3-c3ccc4sc5ccccc5c4c3)c3cc4ccccc4c4ccccc34)cc2)cc1 ; CC1(C)c2ccccc2-c2ccc(N(c3ccc4sc5ccccc5c4c3-c3ccc4sc5ccccc5c4c3)c3cc4ccccc4c4ccccc34)cc21 ; c1ccc(-c2ccc(N(c3ccc(-c4cc5ccccc5c5ccccc45)cc3)c3ccc4sc5ccccc5c4c3-c3ccc4sc5ccccc5c4c3)cc2)cc1]

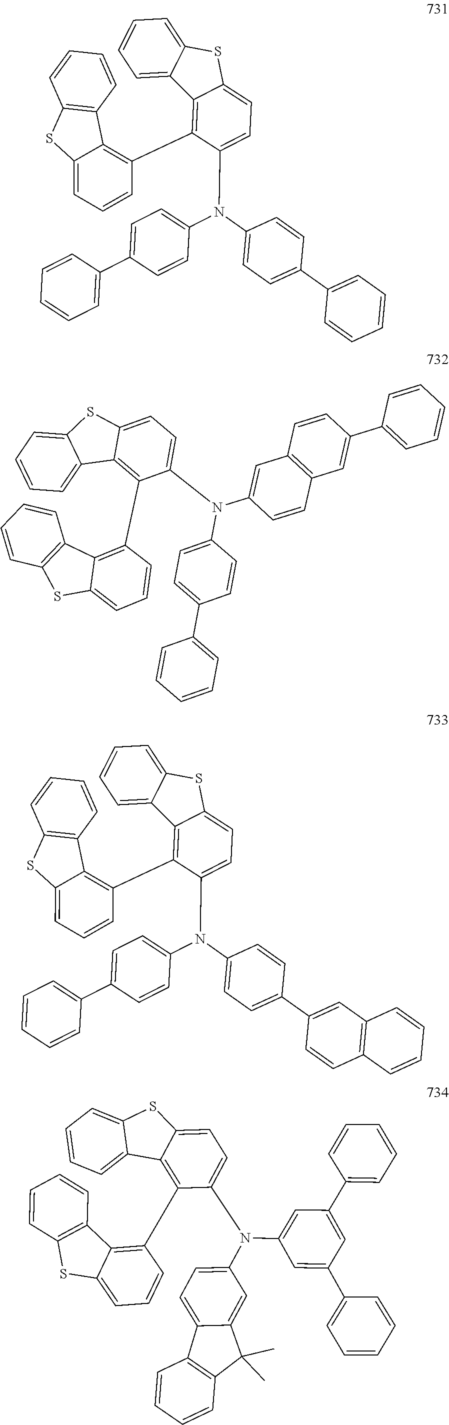 [SMILES: c1ccc(-c2ccc(N(c3ccc(-c4ccccc4)cc3)c3ccc4sc5ccccc5c4c3-c3cccc4sc5ccccc5c34)cc2)cc1 ; c1ccc(-c2ccc(N(c3ccc4cc(-c5ccccc5)ccc4c3)c3ccc4sc5ccccc5c4c3-c3cccc4sc5ccccc5c34)cc2)cc1 ; c1ccc(-c2ccc(N(c3ccc(-c4ccc5ccccc5c4)cc3)c3ccc4sc5ccccc5c4c3-c3cccc4sc5ccccc5c34)cc2)cc1 ; CC1(C)c2ccccc2-c2ccc(N(c3cc(-c4ccccc4)cc(-c4ccccc4)c3)c3ccc4sc5ccccc5c4c3-c3cccc4sc5ccccc5c34)cc21]

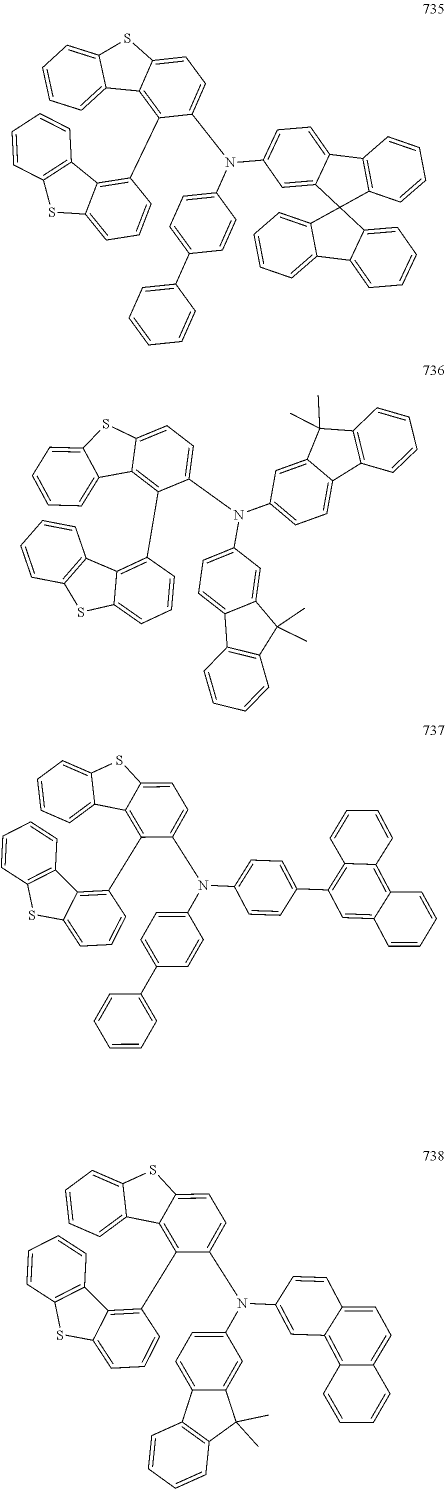 [SMILES: c1ccc(-c2ccc(N(c3ccc4c(c3)C3(c5ccccc5-c5ccccc53)c3ccccc3-4)c3ccc4sc5ccccc5c4c3-c3cccc4sc5ccccc5c34)cc2)cc1 ; CC1(C)c2ccccc2-c2ccc(N(c3ccc4c(c3)C(C)(C)c3ccccc3-4)c3ccc4sc5ccccc5c4c3-c3cccc4sc5ccccc5c34)cc21 ; c1ccc(-c2ccc(N(c3ccc(-c4cc5ccccc5c5ccccc45)cc3)c3ccc4sc5ccccc5c4c3-c3cccc4sc5ccccc5c34)cc2)cc1 ; CC1(C)c2ccccc2-c2ccc(N(c3ccc4ccc5ccccc5c4c3)c3ccc4sc5ccccc5c4c3-c3cccc4sc5ccccc5c34)cc21]

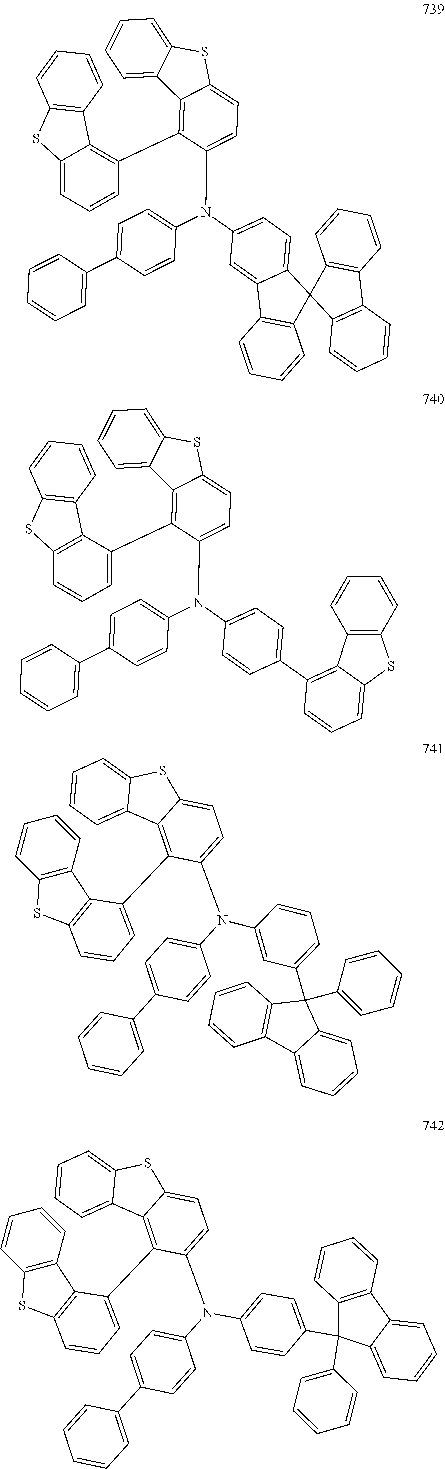 [SMILES: c1ccc(-c2ccc(N(c3ccc4c(c3)-c3ccccc3C43c4ccccc4-c4ccccc43)c3ccc4sc5ccccc5c4c3-c3cccc4sc5ccccc5c34)cc2)cc1 ; c1ccc(-c2ccc(N(c3ccc(-c4cccc5sc6ccccc6c45)cc3)c3ccc4sc5ccccc5c4c3-c3cccc4sc5ccccc5c34)cc2)cc1 ; c1ccc(-c2ccc(N(c3cccc(C4(c5ccccc5)c5ccccc5-c5ccccc54)c3)c3ccc4sc5ccccc5c4c3-c3cccc4sc5ccccc5c34)cc2)cc1 ; c1ccc(-c2ccc(N(c3ccc(C4(c5ccccc5)c5ccccc5-c5ccccc54)cc3)c3ccc4sc5ccccc5c4c3-c3cccc4sc5ccccc5c34)cc2)cc1]

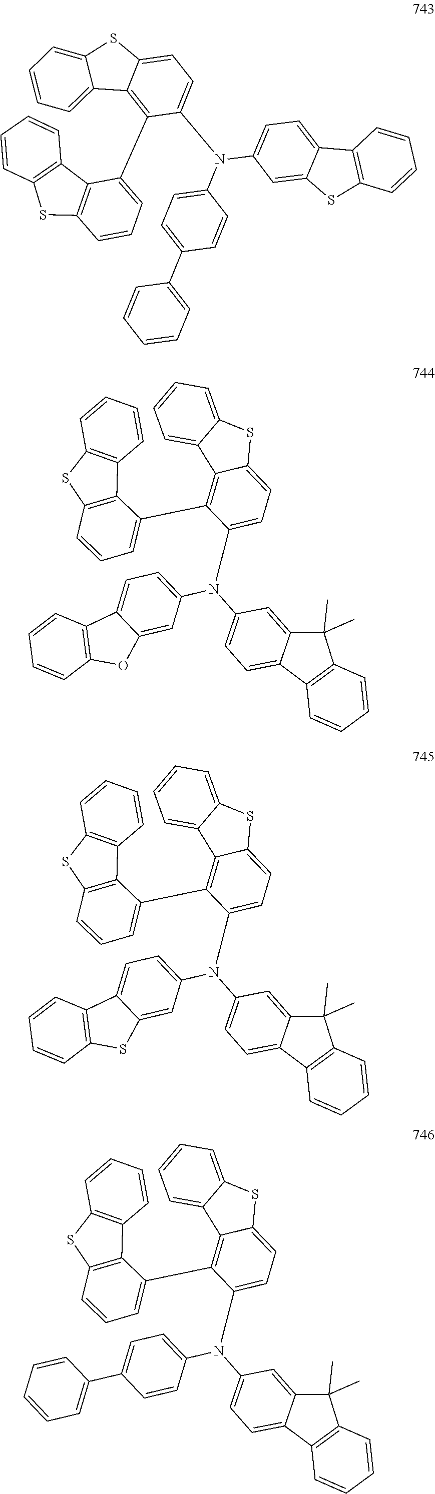

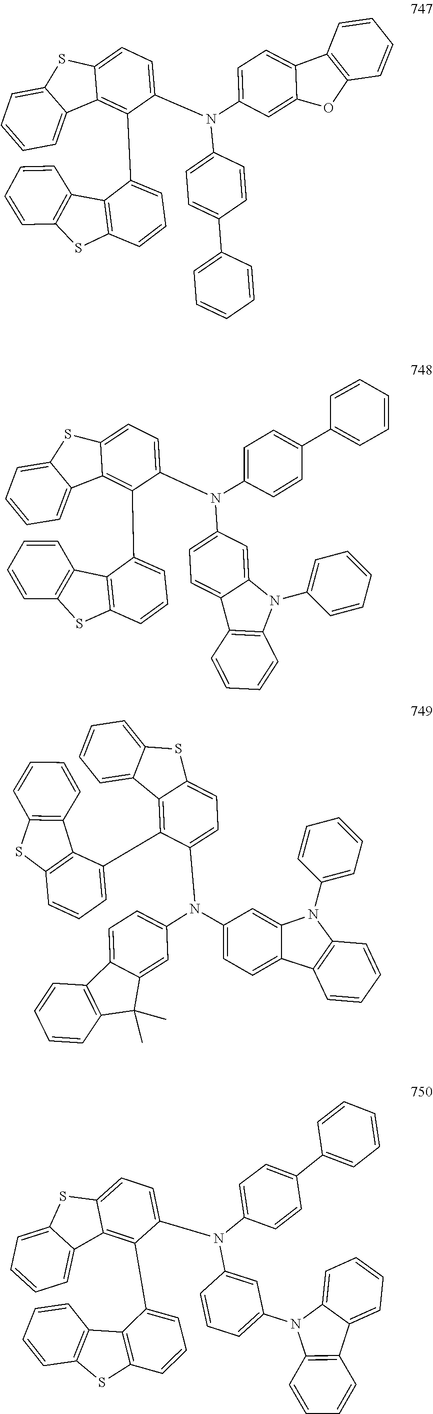 [SMILES: c1ccc(-c2ccc(N(c3ccc4c(c3)oc3ccccc34)c3ccc4sc5ccccc5c4c3-c3cccc4sc5ccccc5c34)cc2)cc1 ; c1ccc(-c2ccc(N(c3ccc4c5ccccc5n(-c5ccccc5)c4c3)c3ccc4sc5ccccc5c4c3-c3cccc4sc5ccccc5c34)cc2)cc1 ; CC1(C)c2ccccc2-c2ccc(N(c3ccc4c5ccccc5n(-c5ccccc5)c4c3)c3ccc4sc5ccccc5c4c3-c3cccc4sc5ccccc5c34)cc21 ; c1ccc(-c2ccc(N(c3cccc(-n4c5ccccc5c5ccccc54)c3)c3ccc4sc5ccccc5c4c3-c3cccc4sc5ccccc5c34)cc2)cc1]

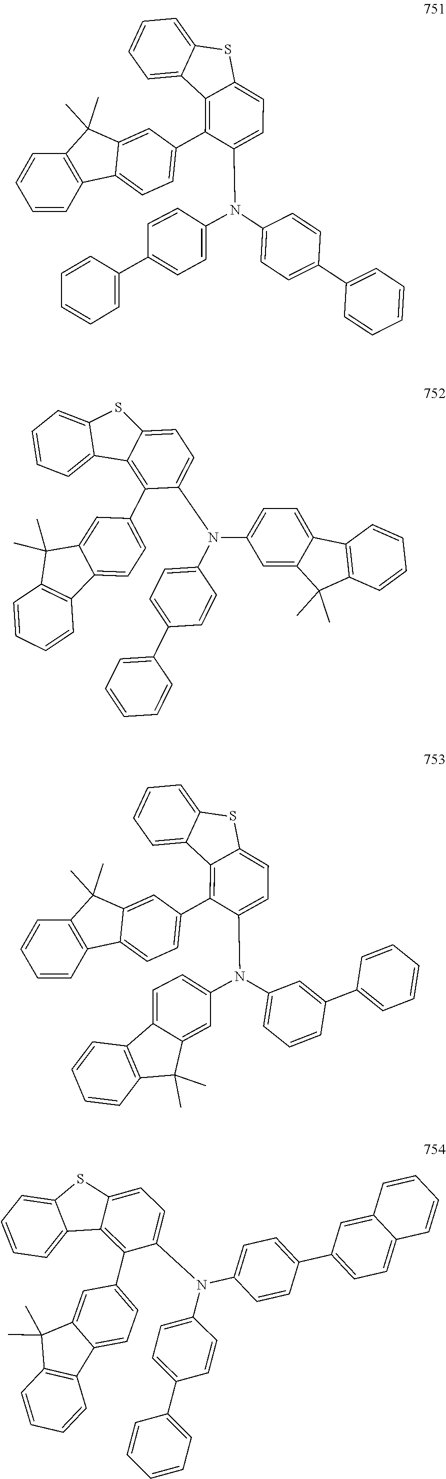 [SMILES: CC1(C)c2ccccc2-c2ccc(-c3c(N(c4ccc(-c5ccccc5)cc4)c4ccc(-c5ccccc5)cc4)ccc4sc5ccccc5c34)cc21 ; CC1(C)c2ccccc2-c2ccc(-c3c(N(c4ccc(-c5ccccc5)cc4)c4ccc5c(c4)C(C)(C)c4ccccc4-5)ccc4sc5ccccc5c34)cc21 ; CC1(C)c2ccccc2-c2ccc(-c3c(N(c4cccc(-c5ccccc5)c4)c4ccc5c(c4)C(C)(C)c4ccccc4-5)ccc4sc5ccccc5c34)cc21 ; CC1(C)c2ccccc2-c2ccc(-c3c(N(c4ccc(-c5ccccc5)cc4)c4ccc(-c5ccc6ccccc6c5)cc4)ccc4sc5ccccc5c34)cc21]

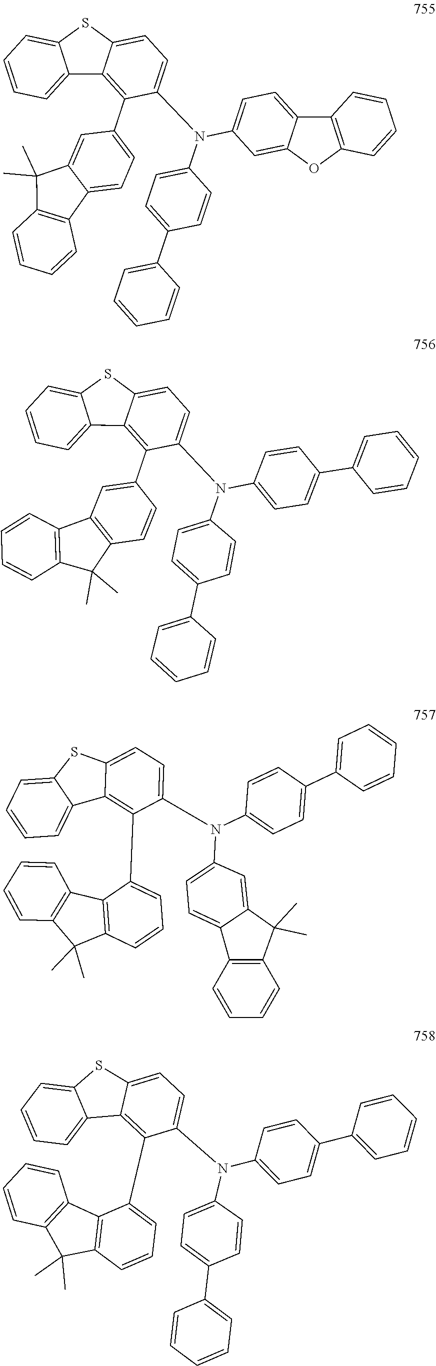 [SMILES: CC1(C)c2ccccc2-c2ccc(-c3c(N(c4ccc(-c5ccccc5)cc4)c4ccc5c(c4)oc4ccccc45)ccc4sc5ccccc5c34)cc21 ; CC1(C)c2ccccc2-c2cc(-c3c(N(c4ccc(-c5ccccc5)cc4)c4ccc(-c5ccccc5)cc4)ccc4sc5ccccc5c34)ccc21 ; CC1(C)c2ccccc2-c2ccc(N(c3ccc(-c4ccccc4)cc3)c3ccc4sc5ccccc5c4c3-c3cccc4c3-c3ccccc3C4(C)C)cc21 ; CC1(C)c2ccccc2-c2c(-c3c(N(c4ccc(-c5ccccc5)cc4)c4ccc(-c5ccccc5)cc4)ccc4sc5ccccc5c34)cccc21]

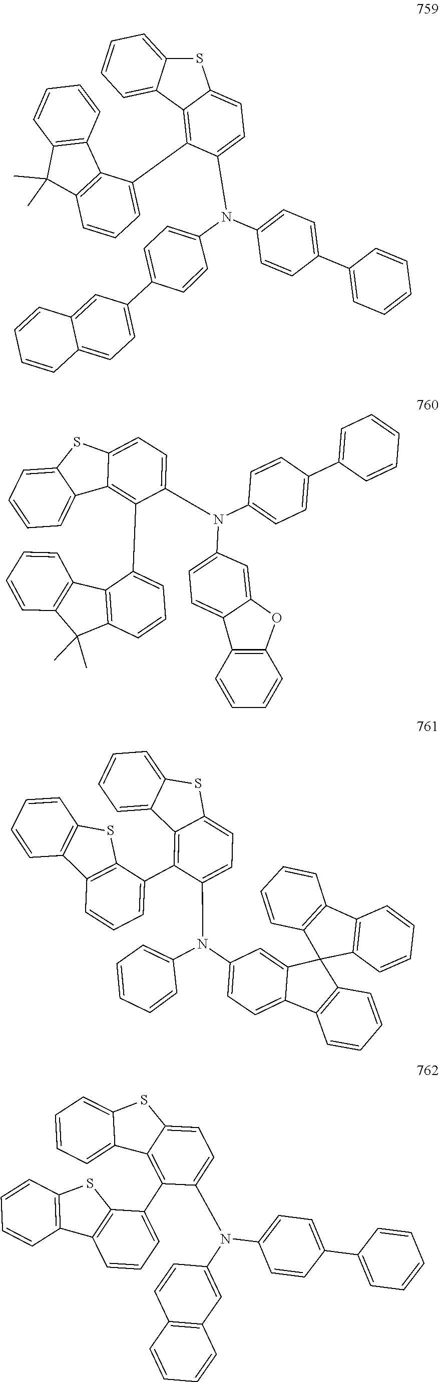 [SMILES: CC1(C)c2ccccc2-c2c(-c3c(N(c4ccc(-c5ccccc5)cc4)c4ccc(-c5ccc6ccccc6c5)cc4)ccc4sc5ccccc5c34)cccc21 ; CC1(C)c2ccccc2-c2c(-c3c(N(c4ccc(-c5ccccc5)cc4)c4ccc5c(c4)oc4ccccc45)ccc4sc5ccccc5c34)cccc21 ; c1ccc(N(c2ccc3c(c2)C2(c4ccccc4-c4ccccc42)c2ccccc2-3)c2ccc3sc4ccccc4c3c2-c2cccc3c2sc2ccccc23)cc1 ; c1ccc(-c2ccc(N(c3ccc4ccccc4c3)c3ccc4sc5ccccc5c4c3-c3cccc4c3sc3ccccc34)cc2)cc1]

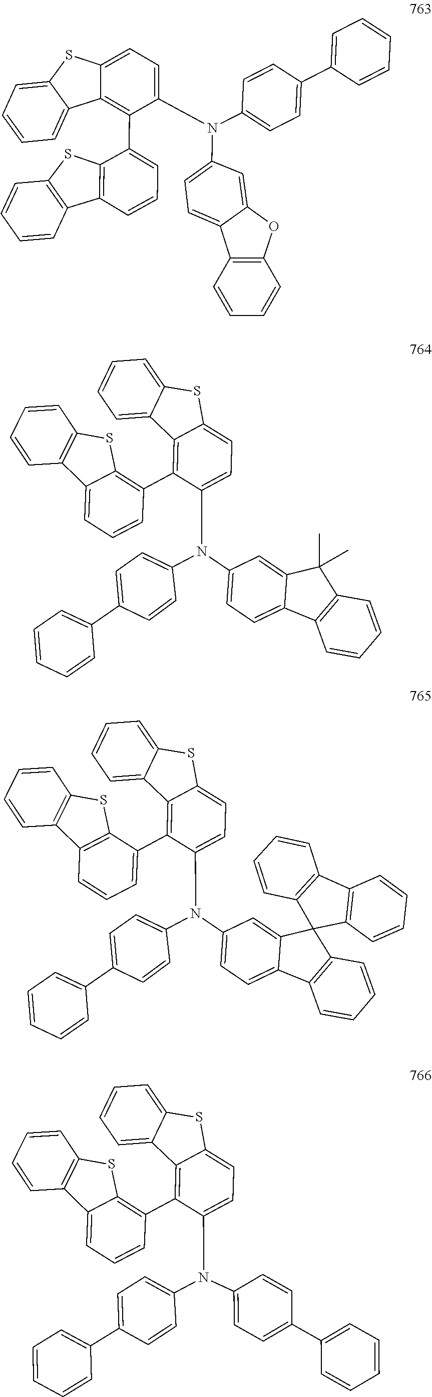 [SMILES: c1ccc(-c2ccc(N(c3ccc4c(c3)oc3ccccc34)c3ccc4sc5ccccc5c4c3-c3cccc4c3sc3ccccc34)cc2)cc1 ; CC1(C)c2ccccc2-c2ccc(N(c3ccc(-c4ccccc4)cc3)c3ccc4sc5ccccc5c4c3-c3cccc4c3sc3ccccc34)cc21 ; c1ccc(-c2ccc(N(c3ccc4c(c3)C3(c5ccccc5-c5ccccc53)c3ccccc3-4)c3ccc4sc5ccccc5c4c3-c3cccc4c3sc3ccccc34)cc2)cc1 ; c1ccc(-c2ccc(N(c3ccc(-c4ccccc4)cc3)c3ccc4sc5ccccc5c4c3-c3cccc4c3sc3ccccc34)cc2)cc1]

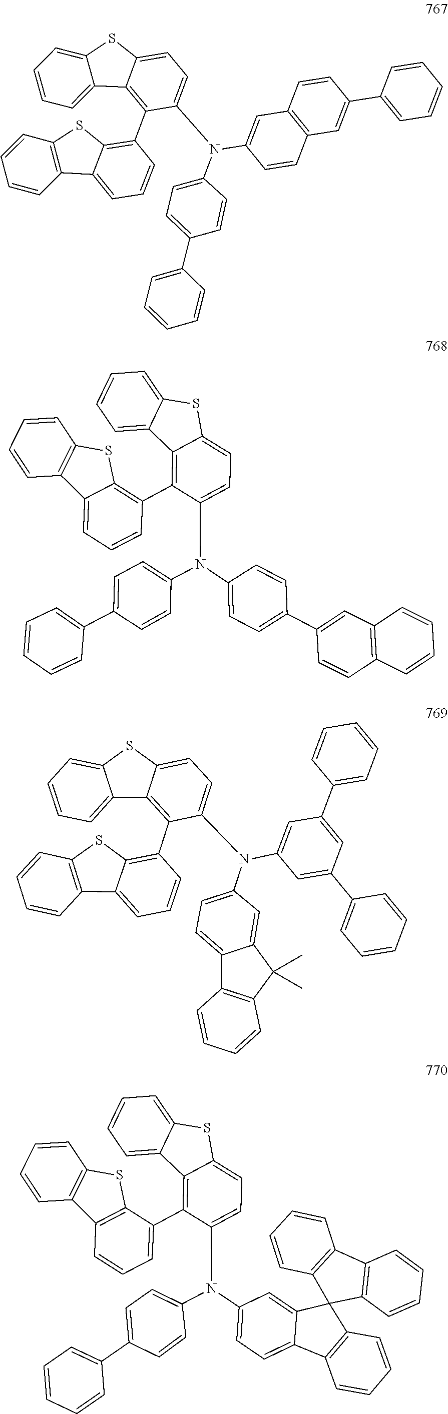

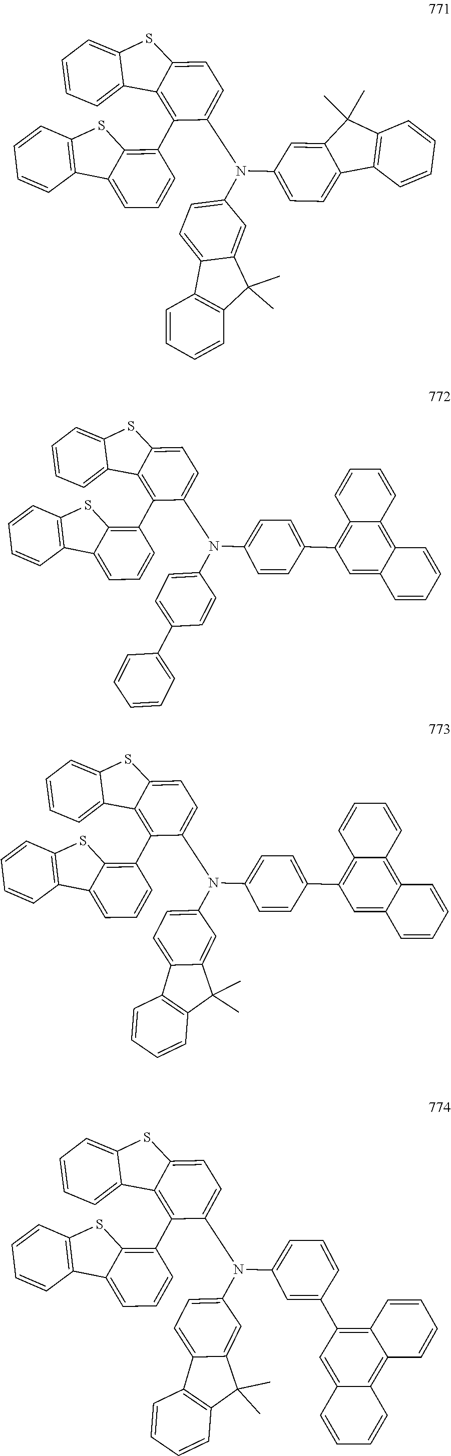 [SMILES: CC1(C)c2ccccc2-c2ccc(N(c3ccc4c(c3)C(C)(C)c3ccccc3-4)c3ccc4sc5ccccc5c4c3-c3cccc4c3sc3ccccc34)cc21 ; c1ccc(-c2ccc(N(c3ccc(-c4cc5ccccc5c5ccccc45)cc3)c3ccc4sc5ccccc5c4c3-c3cccc4c3sc3ccccc34)cc2)cc1 ; CC1(C)c2ccccc2-c2ccc(N(c3ccc(-c4cc5ccccc5c5ccccc45)cc3)c3ccc4sc5ccccc5c4c3-c3cccc4c3sc3ccccc34)cc21 ; CC1(C)c2ccccc2-c2ccc(N(c3cccc(-c4cc5ccccc5c5ccccc45)c3)c3ccc4sc5ccccc5c4c3-c3cccc4c3sc3ccccc34)cc21]

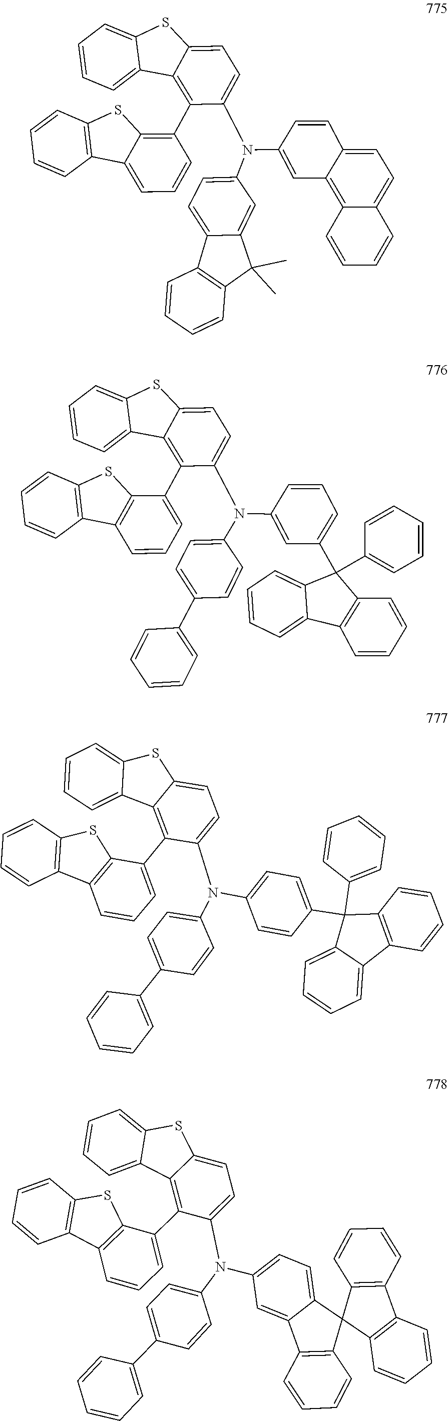 [SMILES: CC1(C)c2ccccc2-c2ccc(N(c3ccc4ccc5ccccc5c4c3)c3ccc4sc5ccccc5c4c3-c3cccc4c3sc3ccccc34)cc21 ; c1ccc(-c2ccc(N(c3cccc(C4(c5ccccc5)c5ccccc5-c5ccccc54)c3)c3ccc4sc5ccccc5c4c3-c3cccc4c3sc3ccccc34)cc2)cc1 ; c1ccc(-c2ccc(N(c3ccc(C4(c5ccccc5)c5ccccc5-c5ccccc54)cc3)c3ccc4sc5ccccc5c4c3-c3cccc4c3sc3ccccc34)cc2)cc1 ; c1ccc(-c2ccc(N(c3ccc4c(c3)-c3ccccc3C43c4ccccc4-c4ccccc43)c3ccc4sc5ccccc5c4c3-c3cccc4c3sc3ccccc34)cc2)cc1]

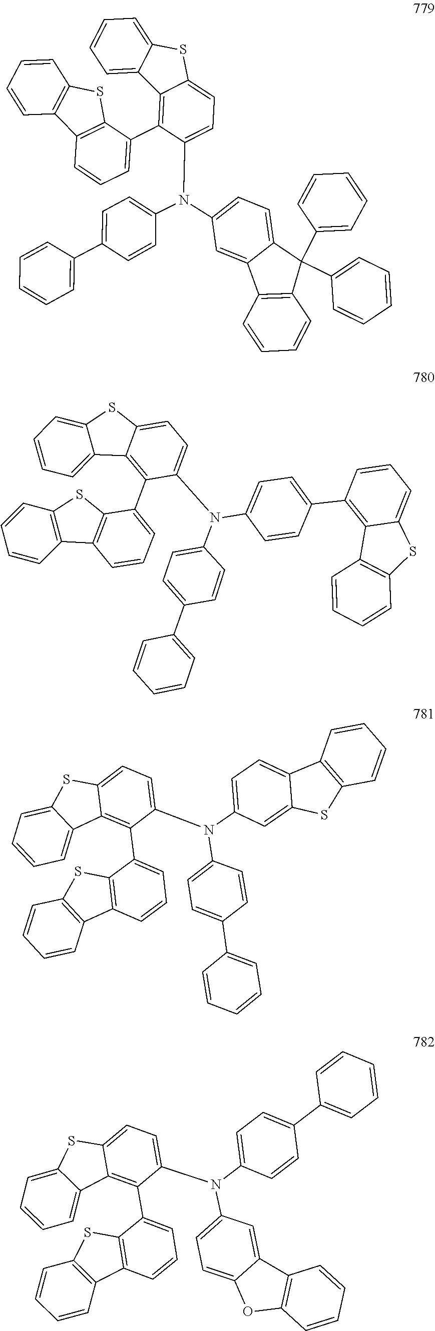 [SMILES: c1ccc(-c2ccc(N(c3ccc4c(c3)-c3ccccc3C4(c3ccccc3)c3ccccc3)c3ccc4sc5ccccc5c4c3-c3cccc4c3sc3ccccc34)cc2)cc1 ; c1ccc(-c2ccc(N(c3ccc(-c4cccc5sc6ccccc6c45)cc3)c3ccc4sc5ccccc5c4c3-c3cccc4c3sc3ccccc34)cc2)cc1 ; c1ccc(-c2ccc(N(c3ccc4c(c3)sc3ccccc34)c3ccc4sc5ccccc5c4c3-c3cccc4c3sc3ccccc34)cc2)cc1 ; c1ccc(-c2ccc(N(c3ccc4oc5ccccc5c4c3)c3ccc4sc5ccccc5c4c3-c3cccc4c3sc3ccccc34)cc2)cc1]

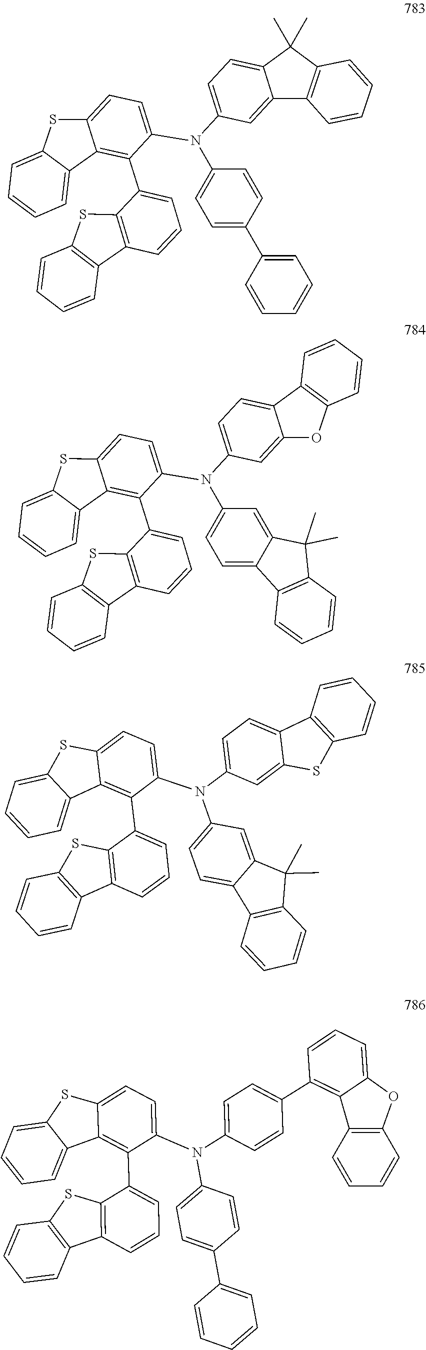

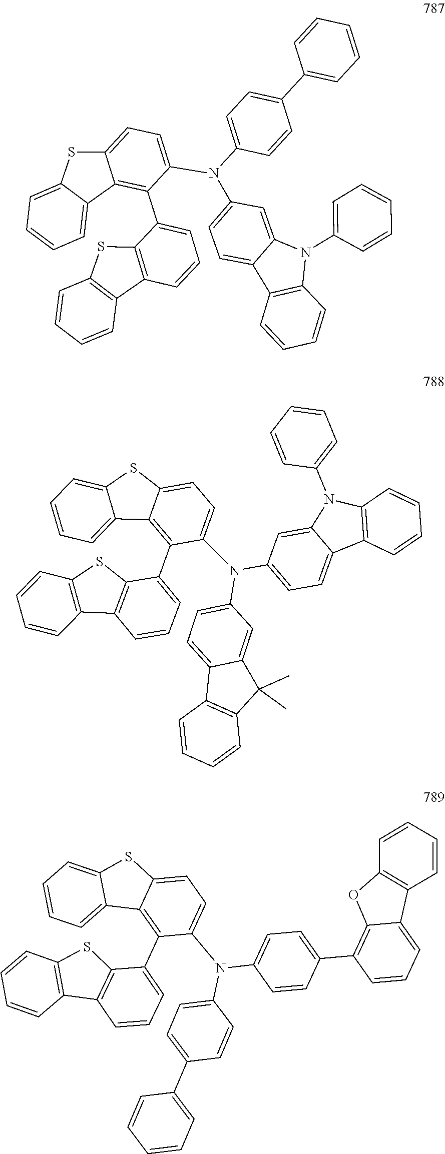 [SMILES: c1ccc(-c2ccc(N(c3ccc4c5ccccc5n(-c5ccccc5)c4c3)c3ccc4sc5ccccc5c4c3-c3cccc4c3sc3ccccc34)cc2)cc1 ; CC1(C)c2ccccc2-c2ccc(N(c3ccc4c5ccccc5n(-c5ccccc5)c4c3)c3ccc4sc5ccccc5c4c3-c3cccc4c3sc3ccccc34)cc21 ; c1ccc(-c2ccc(N(c3ccc(-c4cccc5c4oc4ccccc45)cc3)c3ccc4sc5ccccc5c4c3-c3cccc4c3sc3ccccc34)cc2)cc1]

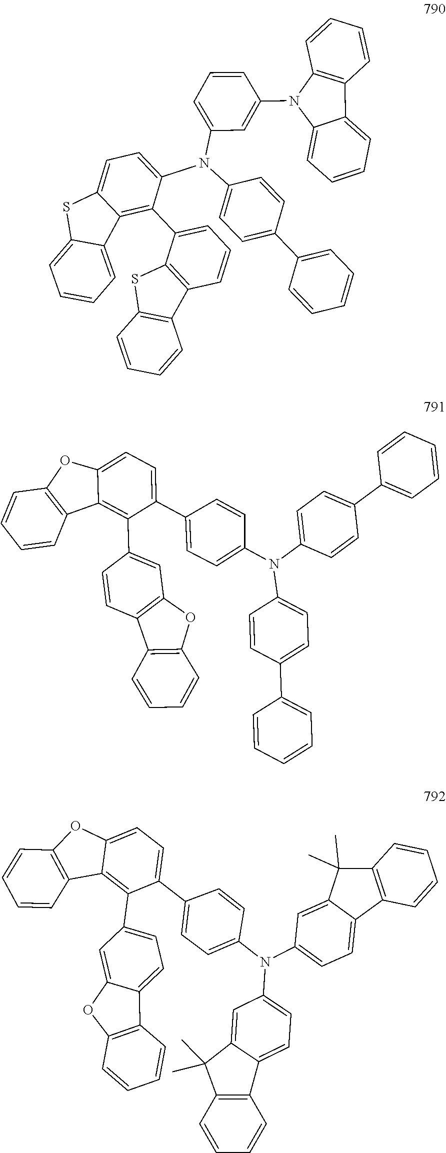 [SMILES: c1ccc(-c2ccc(N(c3cccc(-n4c5ccccc5c5ccccc54)c3)c3ccc4sc5ccccc5c4c3-c3cccc4c3sc3ccccc34)cc2)cc1 ; c1ccc(-c2ccc(N(c3ccc(-c4ccccc4)cc3)c3ccc(-c4ccc5oc6ccccc6c5c4-c4ccc5c(c4)oc4ccccc45)cc3)cc2)cc1 ; CC1(C)c2ccccc2-c2ccc(N(c3ccc(-c4ccc5oc6ccccc6c5c4-c4ccc5c(c4)oc4ccccc45)cc3)c3ccc4c(c3)C(C)(C)c3ccccc3-4)cc21]

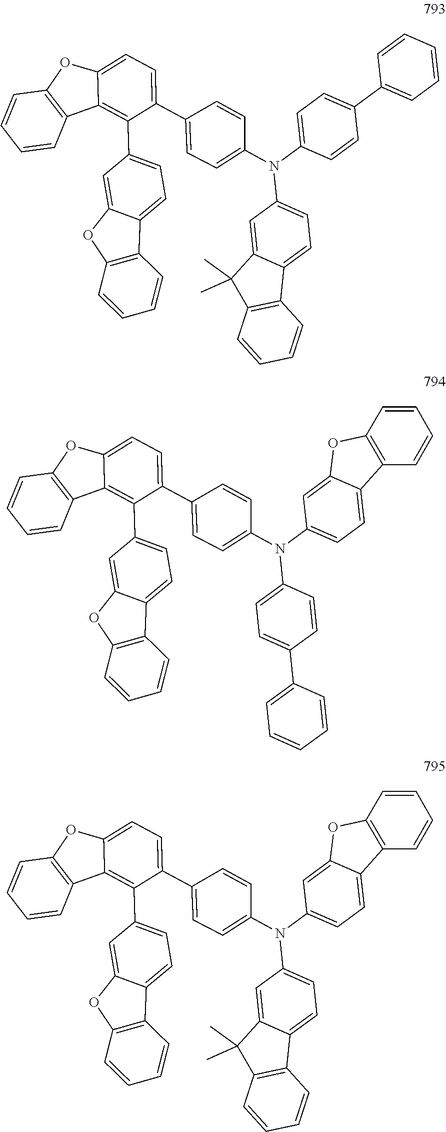 [SMILES: CC1(C)c2ccccc2-c2ccc(N(c3ccc(-c4ccccc4)cc3)c3ccc(-c4ccc5oc6ccccc6c5c4-c4ccc5c(c4)oc4ccccc45)cc3)cc21 ; c1ccc(-c2ccc(N(c3ccc(-c4ccc5oc6ccccc6c5c4-c4ccc5c(c4)oc4ccccc45)cc3)c3ccc4c(c3)oc3ccccc34)cc2)cc1 ; CC1(C)c2ccccc2-c2ccc(N(c3ccc(-c4ccc5oc6ccccc6c5c4-c4ccc5c(c4)oc4ccccc45)cc3)c3ccc4c(c3)oc3ccccc34)cc21]

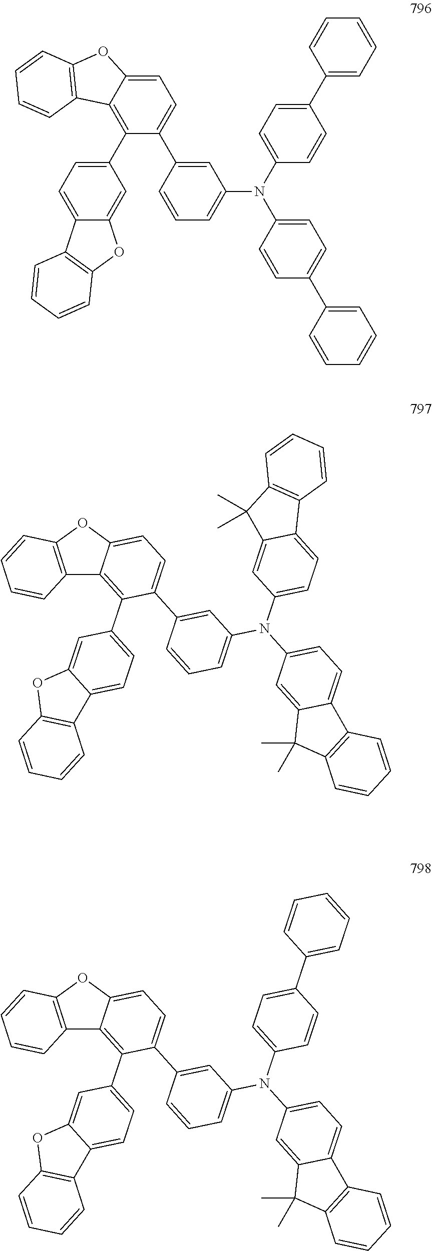 [SMILES: c1ccc(-c2ccc(N(c3ccc(-c4ccccc4)cc3)c3cccc(-c4ccc5oc6ccccc6c5c4-c4ccc5c(c4)oc4ccccc45)c3)cc2)cc1 ; CC1(C)c2ccccc2-c2ccc(N(c3cccc(-c4ccc5oc6ccccc6c5c4-c4ccc5c(c4)oc4ccccc45)c3)c3ccc4c(c3)C(C)(C)c3ccccc3-4)cc21 ; CC1(C)c2ccccc2-c2ccc(N(c3ccc(-c4ccccc4)cc3)c3cccc(-c4ccc5oc6ccccc6c5c4-c4ccc5c(c4)oc4ccccc45)c3)cc21]

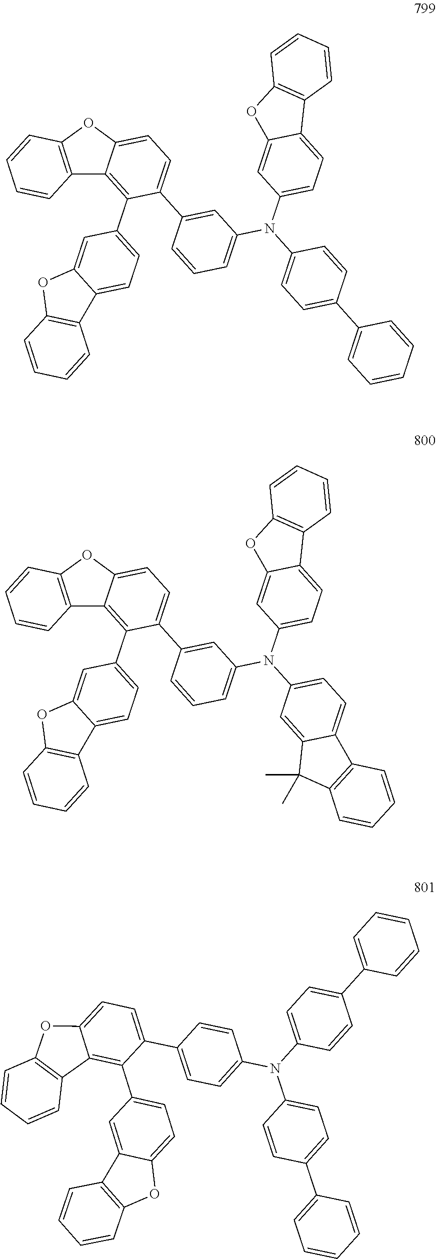 [SMILES: c1ccc(-c2ccc(N(c3cccc(-c4ccc5oc6ccccc6c5c4-c4ccc5c(c4)oc4ccccc45)c3)c3ccc4c(c3)oc3ccccc34)cc2)cc1 ; CC1(C)c2ccccc2-c2ccc(N(c3cccc(-c4ccc5oc6ccccc6c5c4-c4ccc5c(c4)oc4ccccc45)c3)c3ccc4c(c3)oc3ccccc34)cc21 ; c1ccc(-c2ccc(N(c3ccc(-c4ccccc4)cc3)c3ccc(-c4ccc5oc6ccccc6c5c4-c4ccc5oc6ccccc6c5c4)cc3)cc2)cc1]

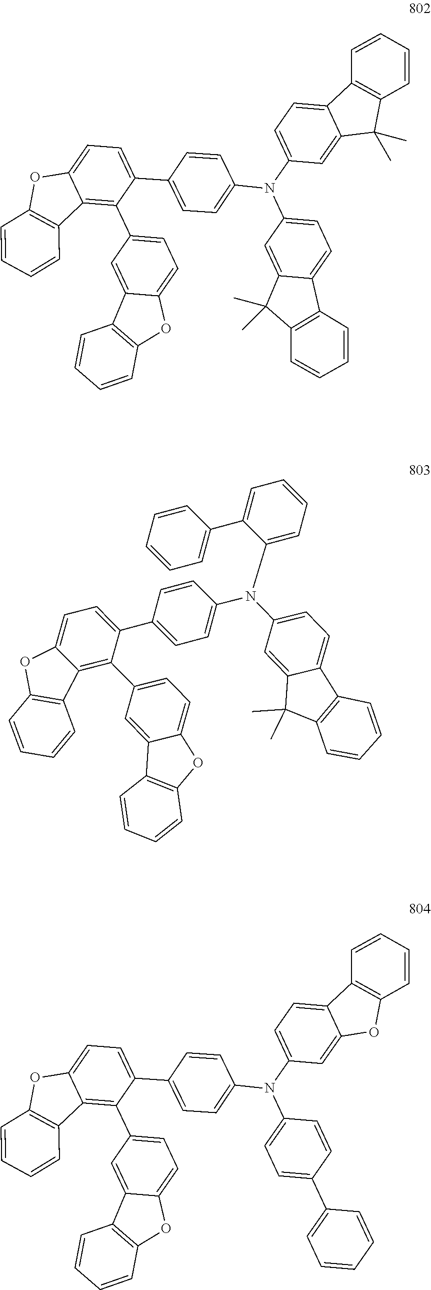 [SMILES: CC1(C)c2ccccc2-c2ccc(N(c3ccc(-c4ccc5oc6ccccc6c5c4-c4ccc5oc6ccccc6c5c4)cc3)c3ccc4c(c3)C(C)(C)c3ccccc3-4)cc21 ; CC1(C)c2ccccc2-c2ccc(N(c3ccc(-c4ccc5oc6ccccc6c5c4-c4ccc5oc6ccccc6c5c4)cc3)c3ccccc3-c3ccccc3)cc21 ; c1ccc(-c2ccc(N(c3ccc(-c4ccc5oc6ccccc6c5c4-c4ccc5oc6ccccc6c5c4)cc3)c3ccc4c(c3)oc3ccccc34)cc2)cc1]

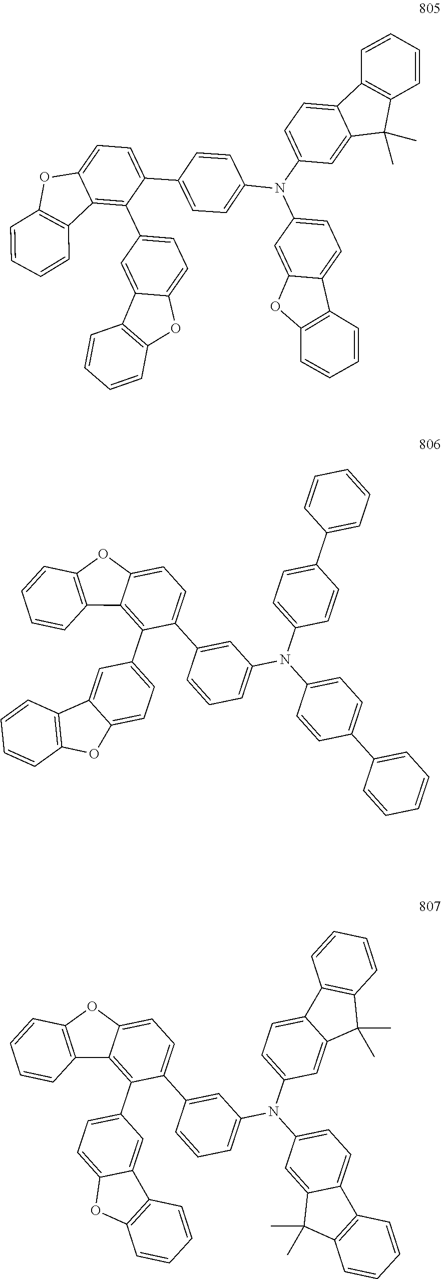 [SMILES: CC1(C)c2ccccc2-c2ccc(N(c3ccc(-c4ccc5oc6ccccc6c5c4-c4ccc5oc6ccccc6c5c4)cc3)c3ccc4c(c3)oc3ccccc34)cc21 ; c1ccc(-c2ccc(N(c3ccc(-c4ccccc4)cc3)c3cccc(-c4ccc5oc6ccccc6c5c4-c4ccc5oc6ccccc6c5c4)c3)cc2)cc1 ; CC1(C)c2ccccc2-c2ccc(N(c3cccc(-c4ccc5oc6ccccc6c5c4-c4ccc5oc6ccccc6c5c4)c3)c3ccc4c(c3)C(C)(C)c3ccccc3-4)cc21]

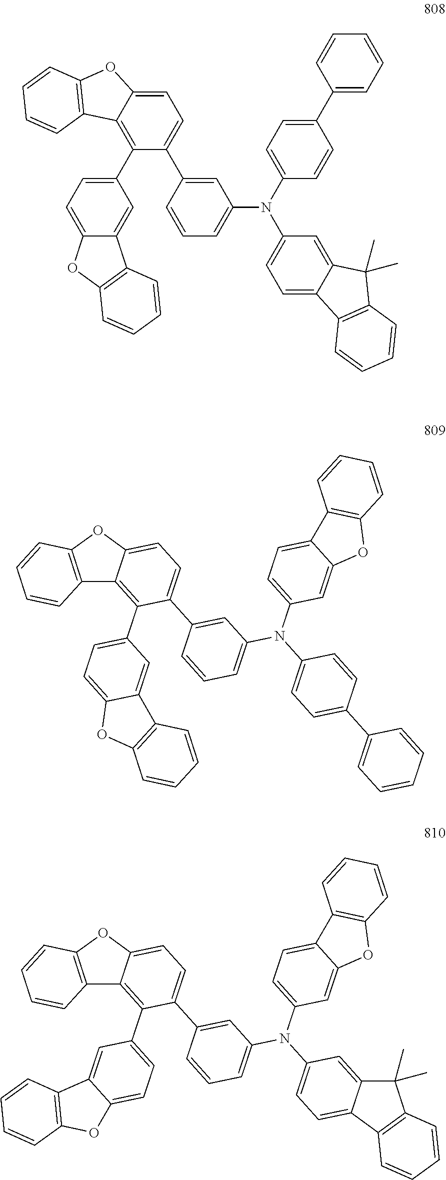 [SMILES: CC1(C)c2ccccc2-c2ccc(N(c3ccc(-c4ccccc4)cc3)c3cccc(-c4ccc5oc6ccccc6c5c4-c4ccc5oc6ccccc6c5c4)c3)cc21 ; c1ccc(-c2ccc(N(c3cccc(-c4ccc5oc6ccccc6c5c4-c4ccc5oc6ccccc6c5c4)c3)c3ccc4c(c3)oc3ccccc34)cc2)cc1 ; CC1(C)c2ccccc2-c2ccc(N(c3cccc(-c4ccc5oc6ccccc6c5c4-c4ccc5oc6ccccc6c5c4)c3)c3ccc4c(c3)oc3ccccc34)cc21]

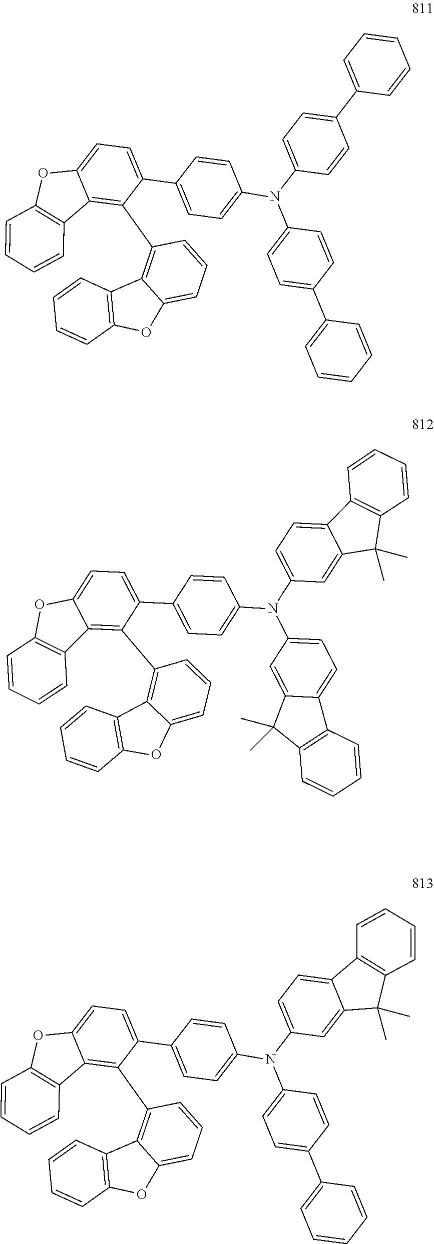 [SMILES: c1ccc(-c2ccc(N(c3ccc(-c4ccccc4)cc3)c3ccc(-c4ccc5oc6ccccc6c5c4-c4cccc5oc6ccccc6c45)cc3)cc2)cc1 ; CC1(C)c2ccccc2-c2ccc(N(c3ccc(-c4ccc5oc6ccccc6c5c4-c4cccc5oc6ccccc6c45)cc3)c3ccc4c(c3)C(C)(C)c3ccccc3-4)cc21 ; CC1(C)c2ccccc2-c2ccc(N(c3ccc(-c4ccccc4)cc3)c3ccc(-c4ccc5oc6ccccc6c5c4-c4cccc5oc6ccccc6c45)cc3)cc21]

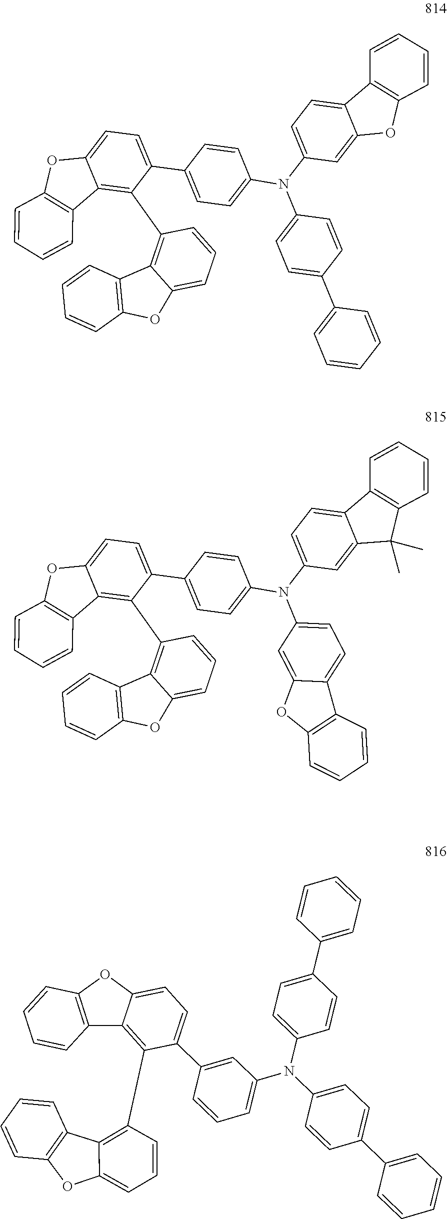 [SMILES: c1ccc(-c2ccc(N(c3ccc(-c4ccc5oc6ccccc6c5c4-c4cccc5oc6ccccc6c45)cc3)c3ccc4c(c3)oc3ccccc34)cc2)cc1 ; CC1(C)c2ccccc2-c2ccc(N(c3ccc(-c4ccc5oc6ccccc6c5c4-c4cccc5oc6ccccc6c45)cc3)c3ccc4c(c3)oc3ccccc34)cc21 ; c1ccc(-c2ccc(N(c3ccc(-c4ccccc4)cc3)c3cccc(-c4ccc5oc6ccccc6c5c4-c4cccc5oc6ccccc6c45)c3)cc2)cc1]

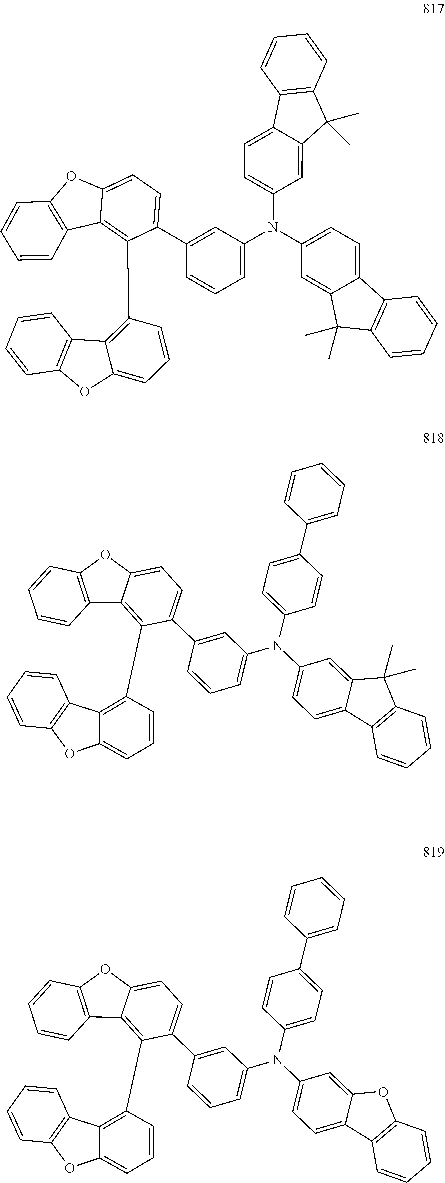 [SMILES: CC1(C)c2ccccc2-c2ccc(N(c3cccc(-c4ccc5oc6ccccc6c5c4-c4cccc5oc6ccccc6c45)c3)c3ccc4c(c3)C(C)(C)c3ccccc3-4)cc21 ; CC1(C)c2ccccc2-c2ccc(N(c3ccc(-c4ccccc4)cc3)c3cccc(-c4ccc5oc6ccccc6c5c4-c4cccc5oc6ccccc6c45)c3)cc21 ; c1ccc(-c2ccc(N(c3cccc(-c4ccc5oc6ccccc6c5c4-c4cccc5oc6ccccc6c45)c3)c3ccc4c(c3)oc3ccccc34)cc2)cc1]

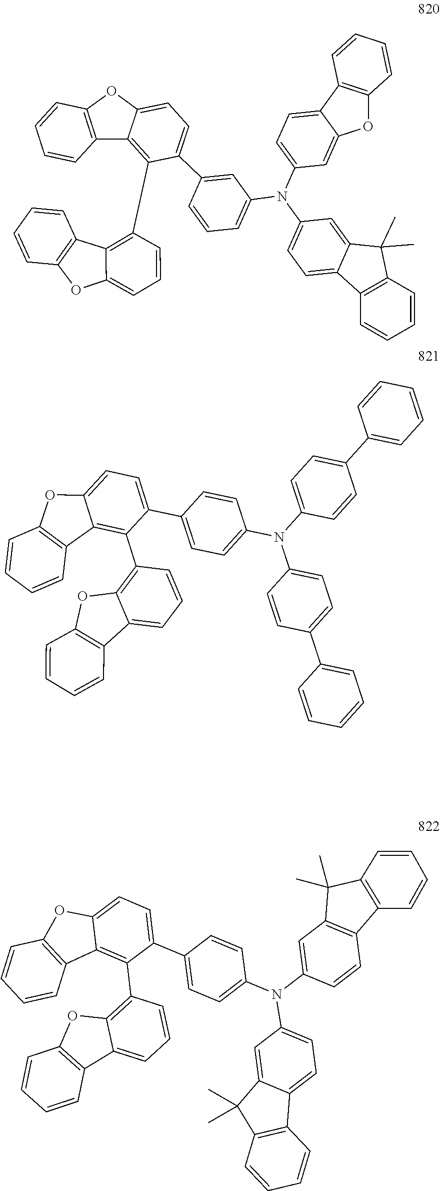

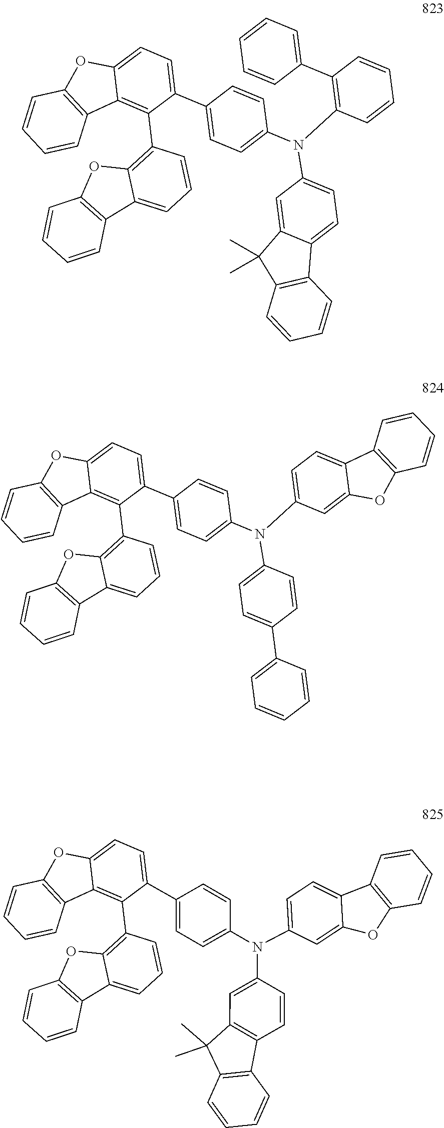 [SMILES: CC1(C)c2ccccc2-c2ccc(N(c3ccc(-c4ccc5oc6ccccc6c5c4-c4cccc5c4oc4ccccc45)cc3)c3ccccc3-c3ccccc3)cc21 ; c1ccc(-c2ccc(N(c3ccc(-c4ccc5oc6ccccc6c5c4-c4cccc5c4oc4ccccc45)cc3)c3ccc4c(c3)oc3ccccc34)cc2)cc1 ; CC1(C)c2ccccc2-c2ccc(N(c3ccc(-c4ccc5oc6ccccc6c5c4-c4cccc5c4oc4ccccc45)cc3)c3ccc4c(c3)oc3ccccc34)cc21]

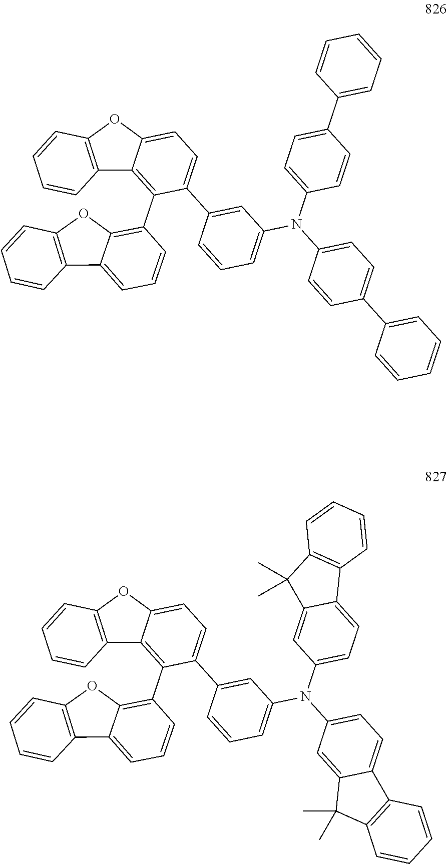 [SMILES: c1ccc(-c2ccc(N(c3ccc(-c4ccccc4)cc3)c3cccc(-c4ccc5oc6ccccc6c5c4-c4cccc5c4oc4ccccc45)c3)cc2)cc1 ; CC1(C)c2ccccc2-c2ccc(N(c3cccc(-c4ccc5oc6ccccc6c5c4-c4cccc5c4oc4ccccc45)c3)c3ccc4c(c3)C(C)(C)c3ccccc3-4)cc21]

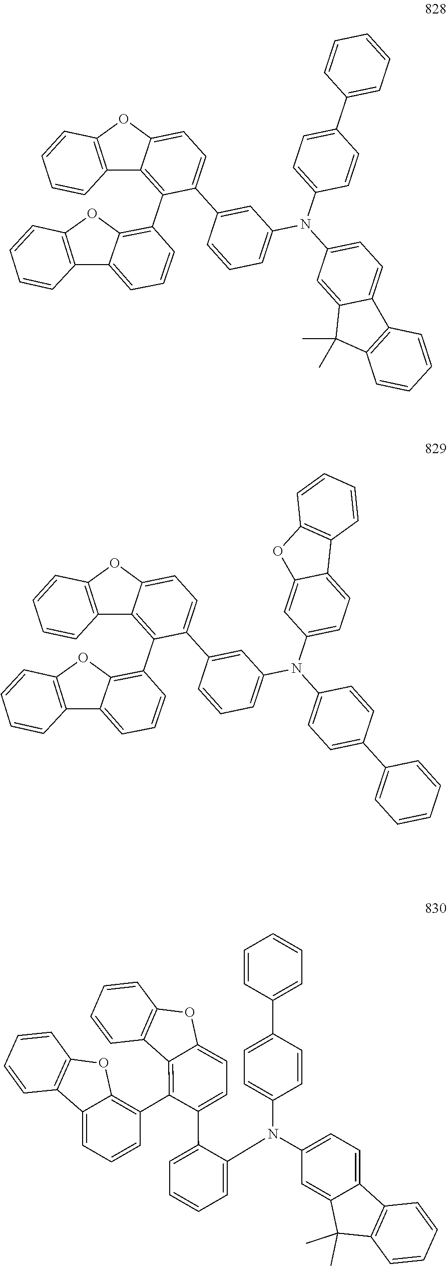 [SMILES: CC1(C)c2ccccc2-c2ccc(N(c3ccc(-c4ccccc4)cc3)c3cccc(-c4ccc5oc6ccccc6c5c4-c4cccc5c4oc4ccccc45)c3)cc21 ; c1ccc(-c2ccc(N(c3cccc(-c4ccc5oc6ccccc6c5c4-c4cccc5c4oc4ccccc45)c3)c3ccc4c(c3)oc3ccccc34)cc2)cc1 ; CC1(C)c2ccccc2-c2ccc(N(c3ccc(-c4ccccc4)cc3)c3ccccc3-c3ccc4oc5ccccc5c4c3-c3cccc4c3oc3ccccc34)cc21]

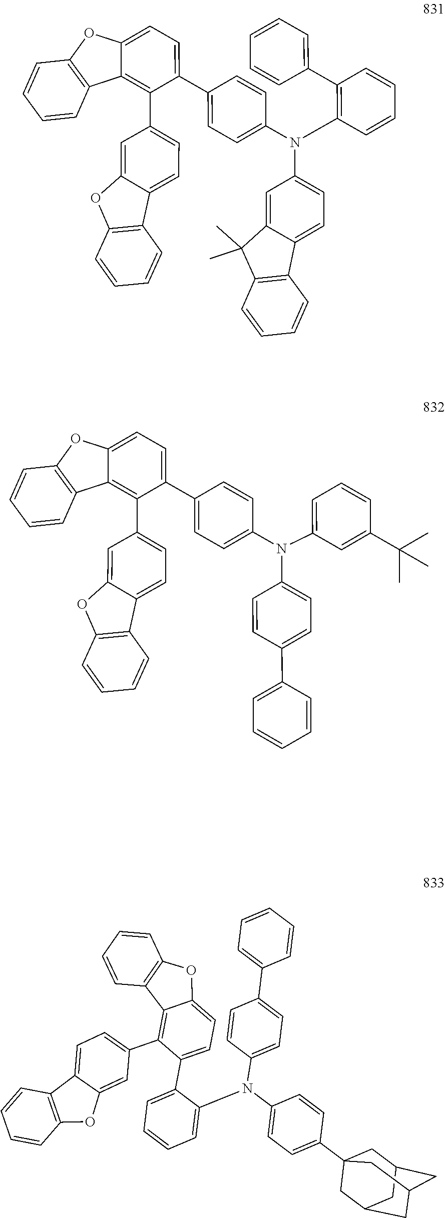 [SMILES: CC1(C)c2ccccc2-c2ccc(N(c3ccc(-c4ccc5oc6ccccc6c5c4-c4ccc5c(c4)oc4ccccc45)cc3)c3ccccc3-c3ccccc3)cc21 ; CC(C)(C)c1cccc(N(c2ccc(-c3ccccc3)cc2)c2ccc(-c3ccc4oc5ccccc5c4c3-c3ccc4c(c3)oc3ccccc34)cc2)c1 ; c1ccc(-c2ccc(N(c3ccc(C45CC6CC(CC(C6)C4)C5)cc3)c3ccccc3-c3ccc4oc5ccccc5c4c3-c3ccc4c(c3)oc3ccccc34)cc2)cc1]

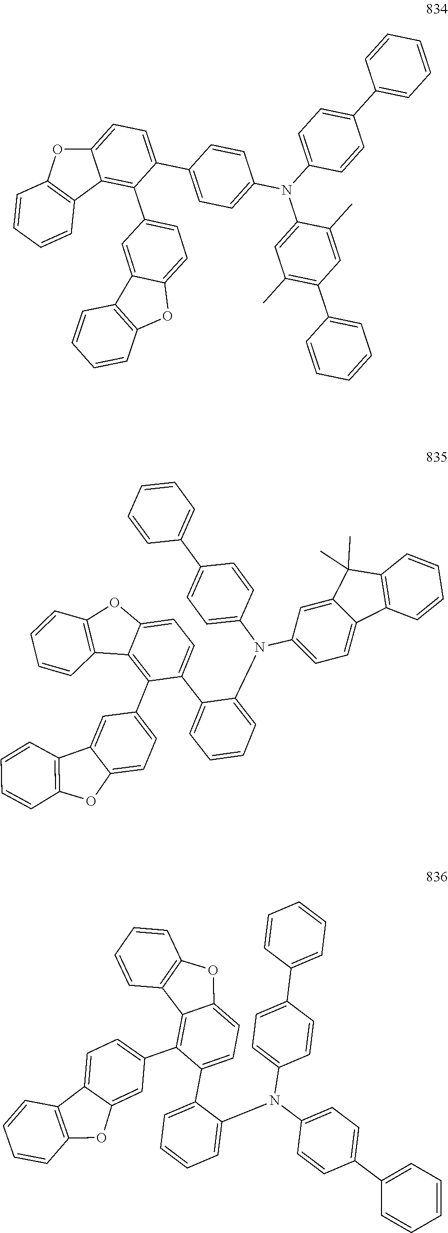 [SMILES: Cc1cc(N(c2ccc(-c3ccccc3)cc2)c2ccc(-c3ccc4oc5ccccc5c4c3-c3ccc4oc5ccccc5c4c3)cc2)c(C)cc1-c1ccccc1 ; CC1(C)c2ccccc2-c2ccc(N(c3ccc(-c4ccccc4)cc3)c3ccccc3-c3ccc4oc5ccccc5c4c3-c3ccc4oc5ccccc5c4c3)cc21 ; c1ccc(-c2ccc(N(c3ccc(-c4ccccc4)cc3)c3ccccc3-c3ccc4oc5ccccc5c4c3-c3ccc4c(c3)oc3ccccc34)cc2)cc1]

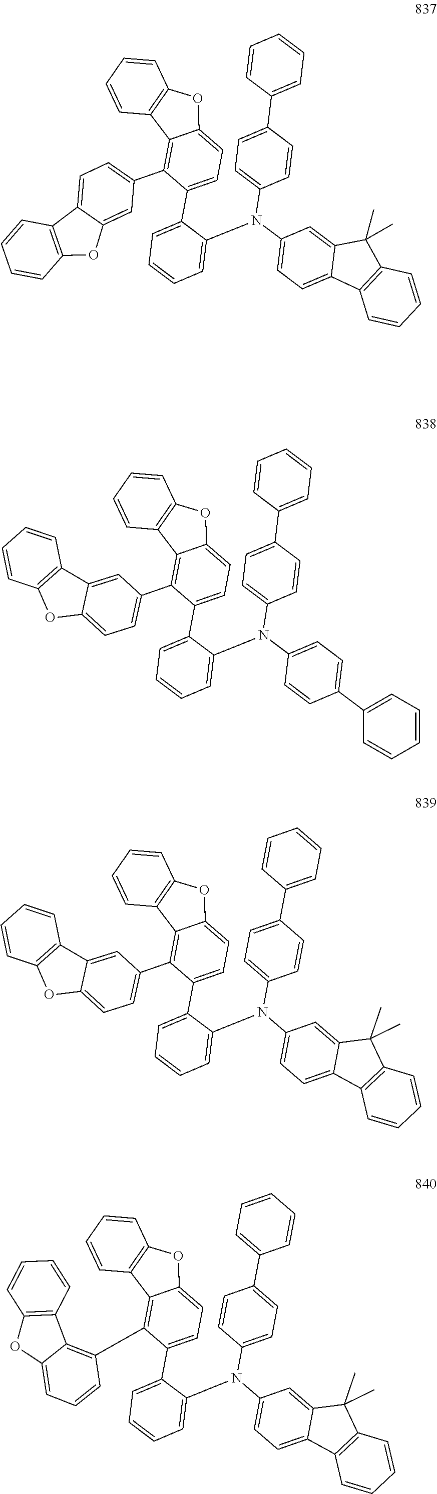 [SMILES: CC1(C)c2ccccc2-c2ccc(N(c3ccc(-c4ccccc4)cc3)c3ccccc3-c3ccc4oc5ccccc5c4c3-c3ccc4c(c3)oc3ccccc34)cc21 ; c1ccc(-c2ccc(N(c3ccc(-c4ccccc4)cc3)c3ccccc3-c3ccc4oc5ccccc5c4c3-c3ccc4oc5ccccc5c4c3)cc2)cc1 ; CC1(C)c2ccccc2-c2ccc(N(c3ccc(-c4ccccc4)cc3)c3ccccc3-c3ccc4oc5ccccc5c4c3-c3ccc4oc5ccccc5c4c3)cc21 ; CC1(C)c2ccccc2-c2ccc(N(c3ccc(-c4ccccc4)cc3)c3ccccc3-c3ccc4oc5ccccc5c4c3-c3cccc4oc5ccccc5c34)cc21]

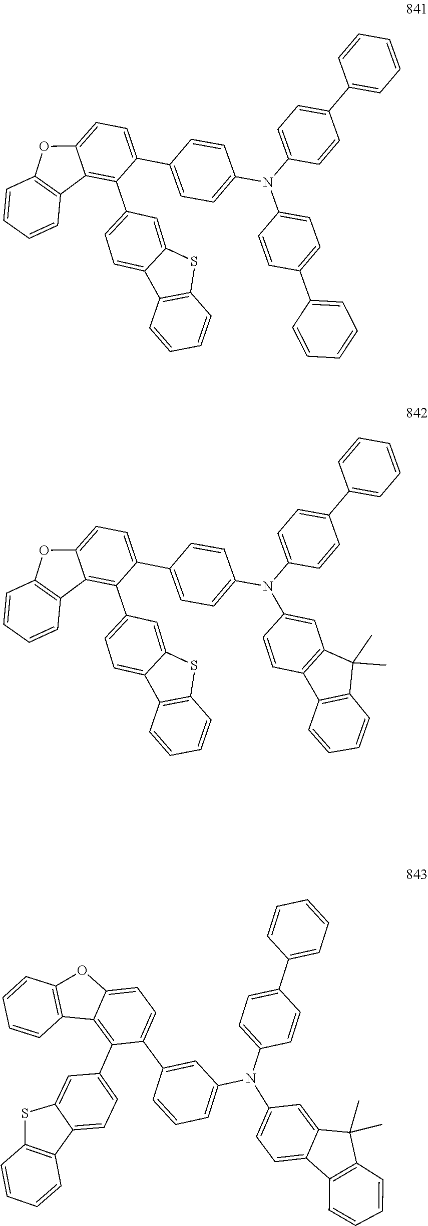 [SMILES: c1ccc(-c2ccc(N(c3ccc(-c4ccccc4)cc3)c3ccc(-c4ccc5oc6ccccc6c5c4-c4ccc5c(c4)sc4ccccc45)cc3)cc2)cc1 ; CC1(C)c2ccccc2-c2ccc(N(c3ccc(-c4ccccc4)cc3)c3ccc(-c4ccc5oc6ccccc6c5c4-c4ccc5c(c4)sc4ccccc45)cc3)cc21 ; CC1(C)c2ccccc2-c2ccc(N(c3ccc(-c4ccccc4)cc3)c3cccc(-c4ccc5oc6ccccc6c5c4-c4ccc5c(c4)sc4ccccc45)c3)cc21]

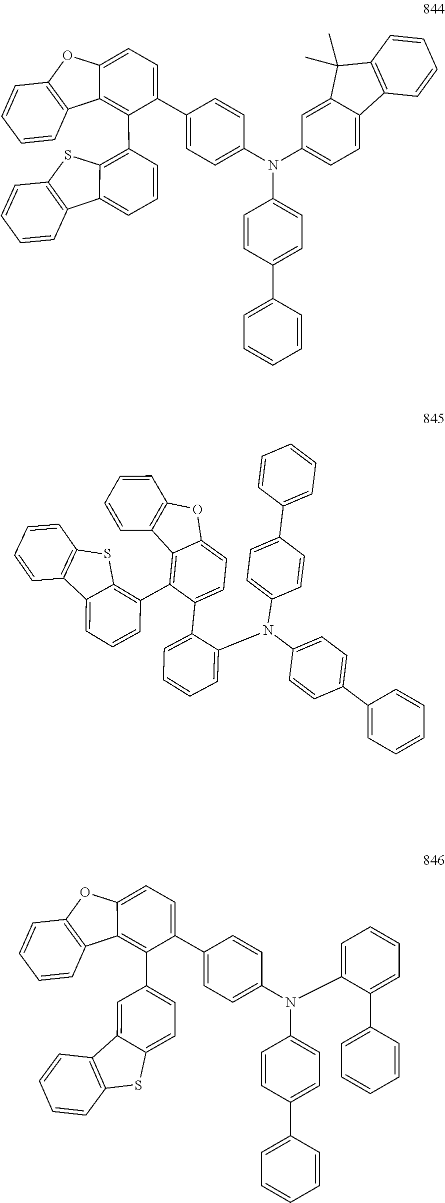 [SMILES: CC1(C)c2ccccc2-c2ccc(N(c3ccc(-c4ccccc4)cc3)c3ccc(-c4ccc5oc6ccccc6c5c4-c4cccc5c4sc4ccccc45)cc3)cc21 ; c1ccc(-c2ccc(N(c3ccc(-c4ccccc4)cc3)c3ccccc3-c3ccc4oc5ccccc5c4c3-c3cccc4c3sc3ccccc34)cc2)cc1 ; c1ccc(-c2ccc(N(c3ccc(-c4ccc5oc6ccccc6c5c4-c4ccc5sc6ccccc6c5c4)cc3)c3ccccc3-c3ccccc3)cc2)cc1]

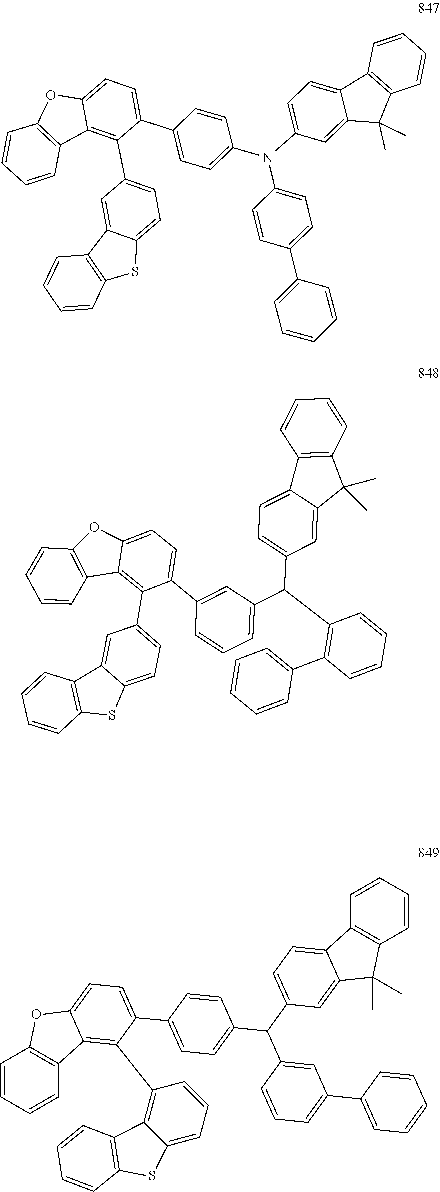 [SMILES: CC1(C)c2ccccc2-c2ccc(N(c3ccc(-c4ccccc4)cc3)c3ccc(-c4ccc5oc6ccccc6c5c4-c4ccc5sc6ccccc6c5c4)cc3)cc21 ; CC1(C)c2ccccc2-c2ccc(C(c3cccc(-c4ccc5oc6ccccc6c5c4-c4ccc5sc6ccccc6c5c4)c3)c3ccccc3-c3ccccc3)cc21 ; CC1(C)c2ccccc2-c2ccc(C(c3ccc(-c4ccc5oc6ccccc6c5c4-c4cccc5sc6ccccc6c45)cc3)c3cccc(-c4ccccc4)c3)cc21]

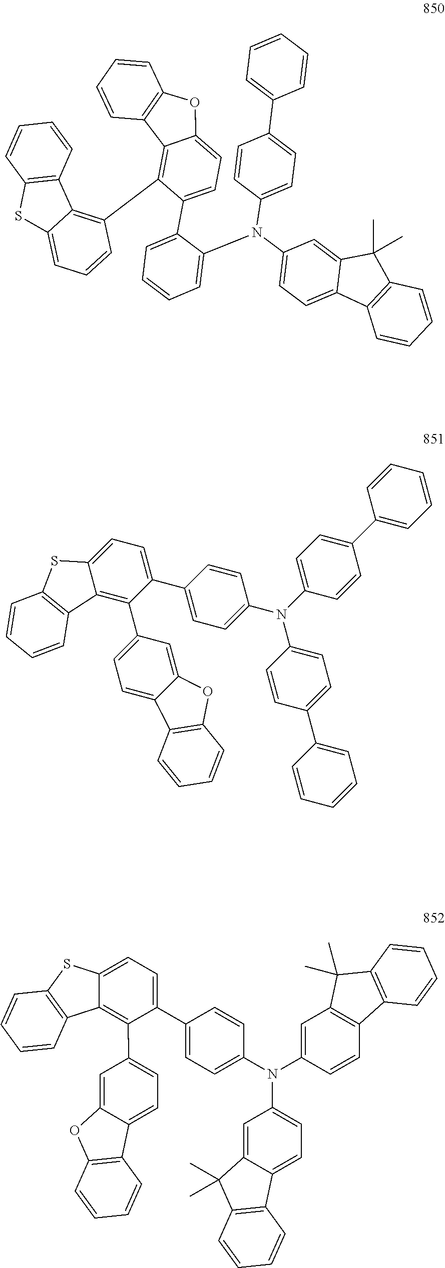 [SMILES: CC1(C)c2ccccc2-c2ccc(N(c3ccc(-c4ccccc4)cc3)c3ccccc3-c3ccc4oc5ccccc5c4c3-c3cccc4sc5ccccc5c34)cc21 ; c1ccc(-c2ccc(N(c3ccc(-c4ccccc4)cc3)c3ccc(-c4ccc5sc6ccccc6c5c4-c4ccc5c(c4)oc4ccccc45)cc3)cc2)cc1 ; CC1(C)c2ccccc2-c2ccc(N(c3ccc(-c4ccc5sc6ccccc6c5c4-c4ccc5c(c4)oc4ccccc45)cc3)c3ccc4c(c3)C(C)(C)c3ccccc3-4)cc21]

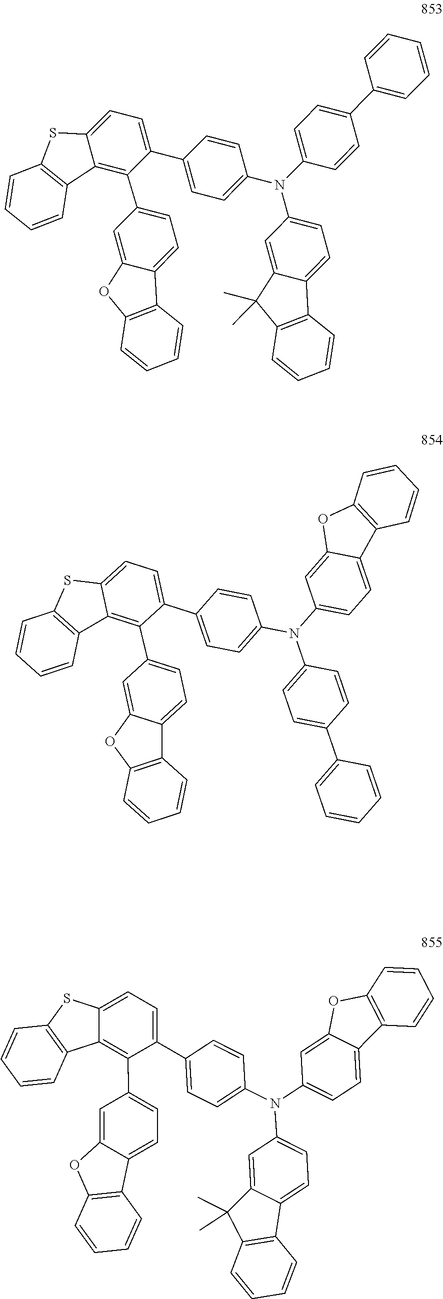 [SMILES: CC1(C)c2ccccc2-c2ccc(N(c3ccc(-c4ccccc4)cc3)c3ccc(-c4ccc5sc6ccccc6c5c4-c4ccc5c(c4)oc4ccccc45)cc3)cc21 ; c1ccc(-c2ccc(N(c3ccc(-c4ccc5sc6ccccc6c5c4-c4ccc5c(c4)oc4ccccc45)cc3)c3ccc4c(c3)oc3ccccc34)cc2)cc1 ; CC1(C)c2ccccc2-c2ccc(N(c3ccc(-c4ccc5sc6ccccc6c5c4-c4ccc5c(c4)oc4ccccc45)cc3)c3ccc4c(c3)oc3ccccc34)cc21]

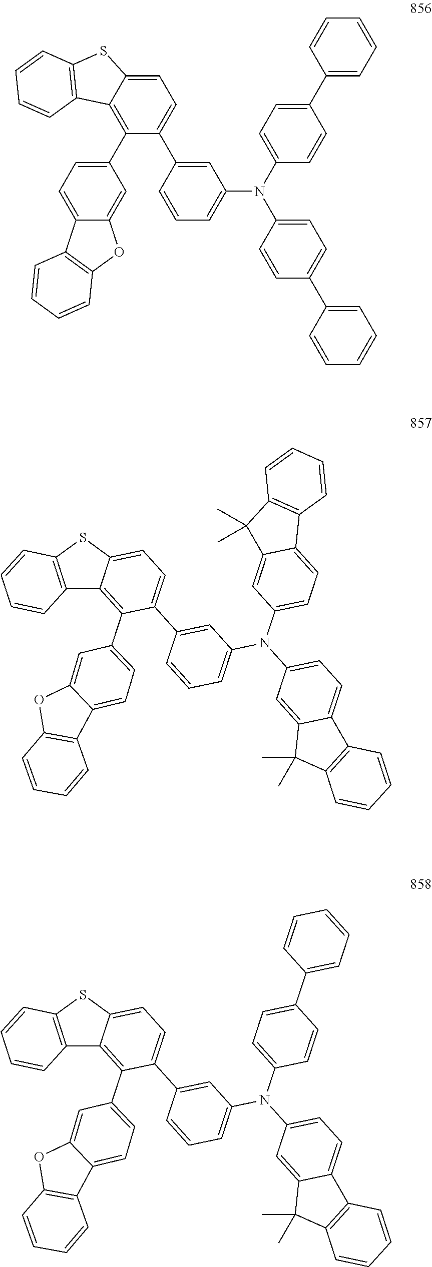 [SMILES: c1ccc(-c2ccc(N(c3ccc(-c4ccccc4)cc3)c3cccc(-c4ccc5sc6ccccc6c5c4-c4ccc5c(c4)oc4ccccc45)c3)cc2)cc1 ; CC1(C)c2ccccc2-c2ccc(N(c3cccc(-c4ccc5sc6ccccc6c5c4-c4ccc5c(c4)oc4ccccc45)c3)c3ccc4c(c3)C(C)(C)c3ccccc3-4)cc21 ; CC1(C)c2ccccc2-c2ccc(N(c3ccc(-c4ccccc4)cc3)c3cccc(-c4ccc5sc6ccccc6c5c4-c4ccc5c(c4)oc4ccccc45)c3)cc21]

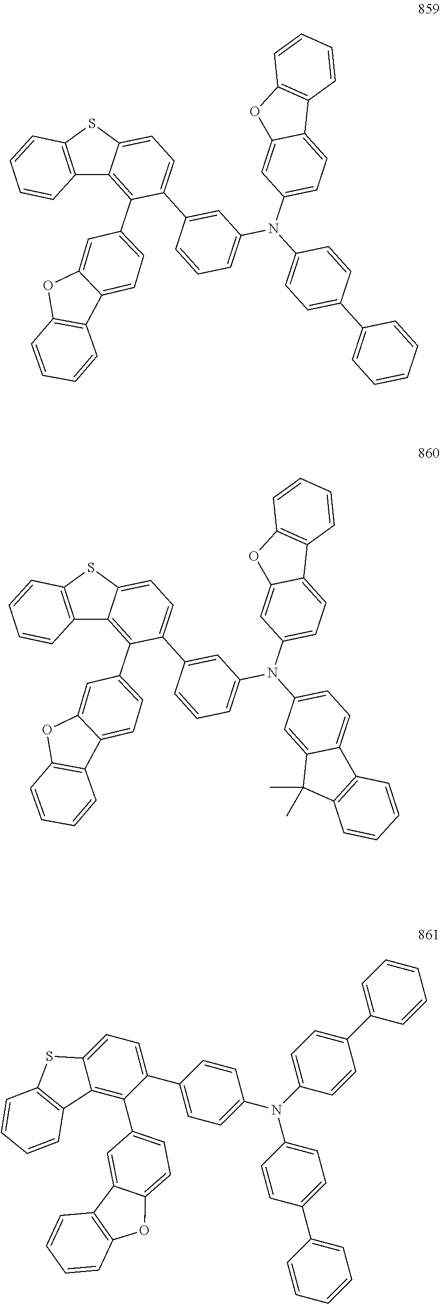 [SMILES: c1ccc(-c2ccc(N(c3cccc(-c4ccc5sc6ccccc6c5c4-c4ccc5c(c4)oc4ccccc45)c3)c3ccc4c(c3)oc3ccccc34)cc2)cc1 ; CC1(C)c2ccccc2-c2ccc(N(c3cccc(-c4ccc5sc6ccccc6c5c4-c4ccc5c(c4)oc4ccccc45)c3)c3ccc4c(c3)oc3ccccc34)cc21 ; c1ccc(-c2ccc(N(c3ccc(-c4ccccc4)cc3)c3ccc(-c4ccc5sc6ccccc6c5c4-c4ccc5oc6ccccc6c5c4)cc3)cc2)cc1]

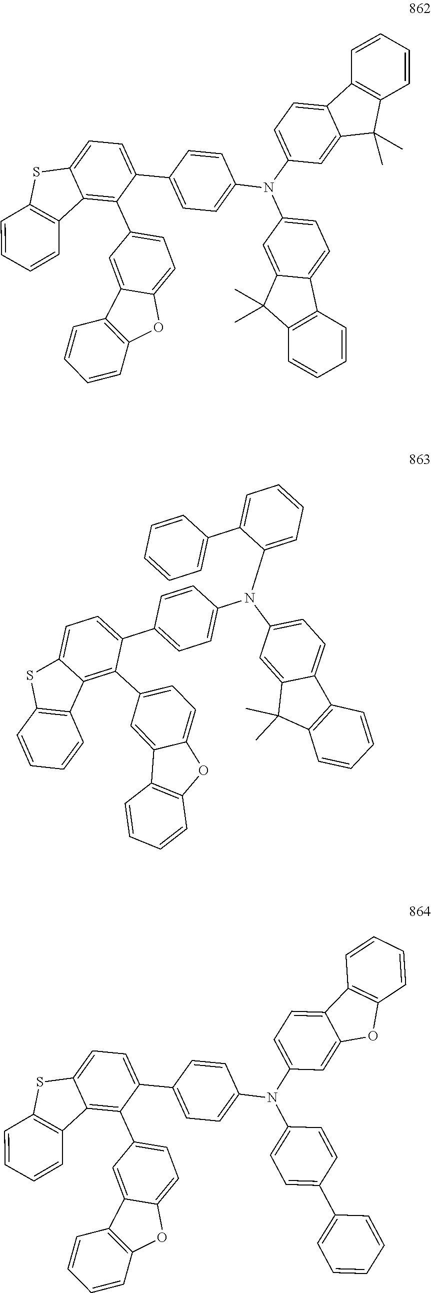 [SMILES: CC1(C)c2ccccc2-c2ccc(N(c3ccc(-c4ccc5sc6ccccc6c5c4-c4ccc5oc6ccccc6c5c4)cc3)c3ccc4c(c3)C(C)(C)c3ccccc3-4)cc21 ; CC1(C)c2ccccc2-c2ccc(N(c3ccc(-c4ccc5sc6ccccc6c5c4-c4ccc5oc6ccccc6c5c4)cc3)c3ccccc3-c3ccccc3)cc21 ; c1ccc(-c2ccc(N(c3ccc(-c4ccc5sc6ccccc6c5c4-c4ccc5oc6ccccc6c5c4)cc3)c3ccc4c(c3)oc3ccccc34)cc2)cc1]

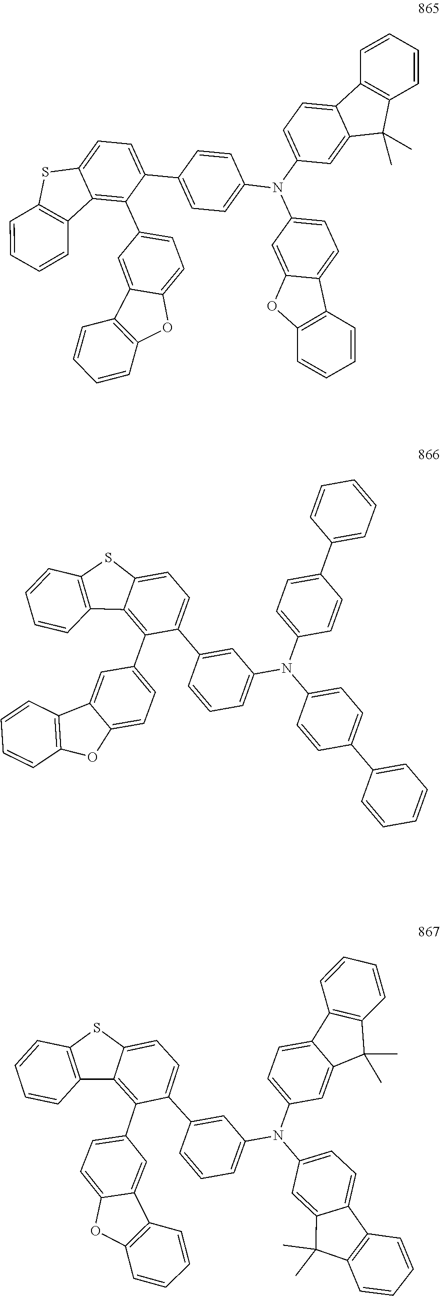 [SMILES: CC1(C)c2ccccc2-c2ccc(N(c3ccc(-c4ccc5sc6ccccc6c5c4-c4ccc5oc6ccccc6c5c4)cc3)c3ccc4c(c3)oc3ccccc34)cc21 ; c1ccc(-c2ccc(N(c3ccc(-c4ccccc4)cc3)c3cccc(-c4ccc5sc6ccccc6c5c4-c4ccc5oc6ccccc6c5c4)c3)cc2)cc1 ; CC1(C)c2ccccc2-c2ccc(N(c3cccc(-c4ccc5sc6ccccc6c5c4-c4ccc5oc6ccccc6c5c4)c3)c3ccc4c(c3)C(C)(C)c3ccccc3-4)cc21]

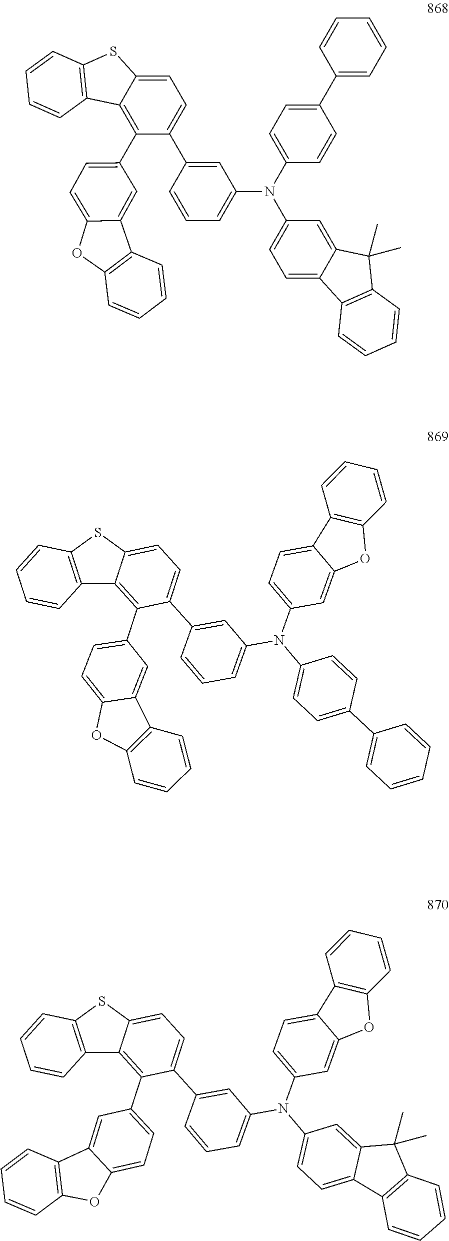 [SMILES: CC1(C)c2ccccc2-c2ccc(N(c3ccc(-c4ccccc4)cc3)c3cccc(-c4ccc5sc6ccccc6c5c4-c4ccc5oc6ccccc6c5c4)c3)cc21 ; c1ccc(-c2ccc(N(c3cccc(-c4ccc5sc6ccccc6c5c4-c4ccc5oc6ccccc6c5c4)c3)c3ccc4c(c3)oc3ccccc34)cc2)cc1 ; CC1(C)c2ccccc2-c2ccc(N(c3cccc(-c4ccc5sc6ccccc6c5c4-c4ccc5oc6ccccc6c5c4)c3)c3ccc4c(c3)oc3ccccc34)cc21]

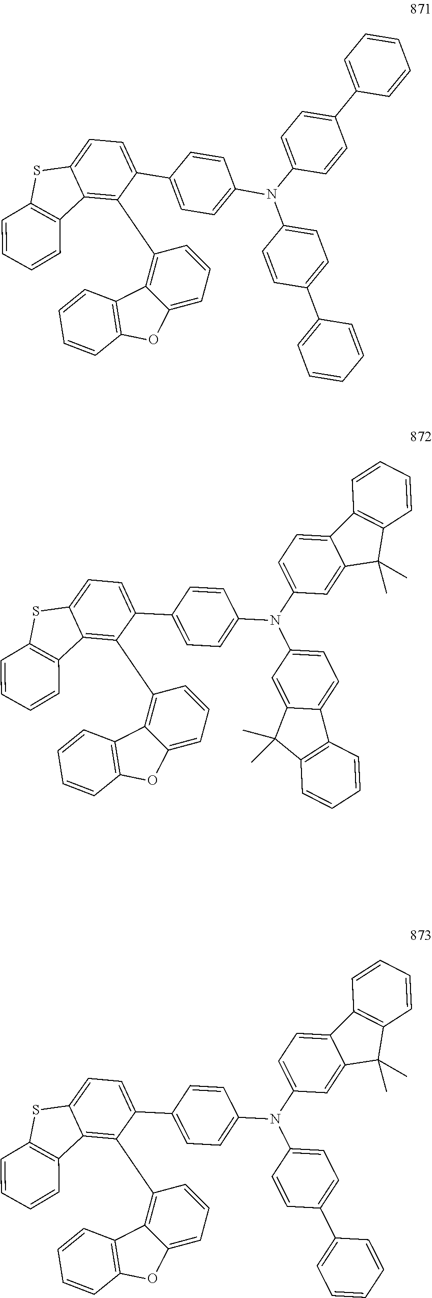 [SMILES: c1ccc(-c2ccc(N(c3ccc(-c4ccccc4)cc3)c3ccc(-c4ccc5sc6ccccc6c5c4-c4cccc5oc6ccccc6c45)cc3)cc2)cc1 ; CC1(C)c2ccccc2-c2ccc(N(c3ccc(-c4ccc5sc6ccccc6c5c4-c4cccc5oc6ccccc6c45)cc3)c3ccc4c(c3)C(C)(C)c3ccccc3-4)cc21 ; CC1(C)c2ccccc2-c2ccc(N(c3ccc(-c4ccccc4)cc3)c3ccc(-c4ccc5sc6ccccc6c5c4-c4cccc5oc6ccccc6c45)cc3)cc21]

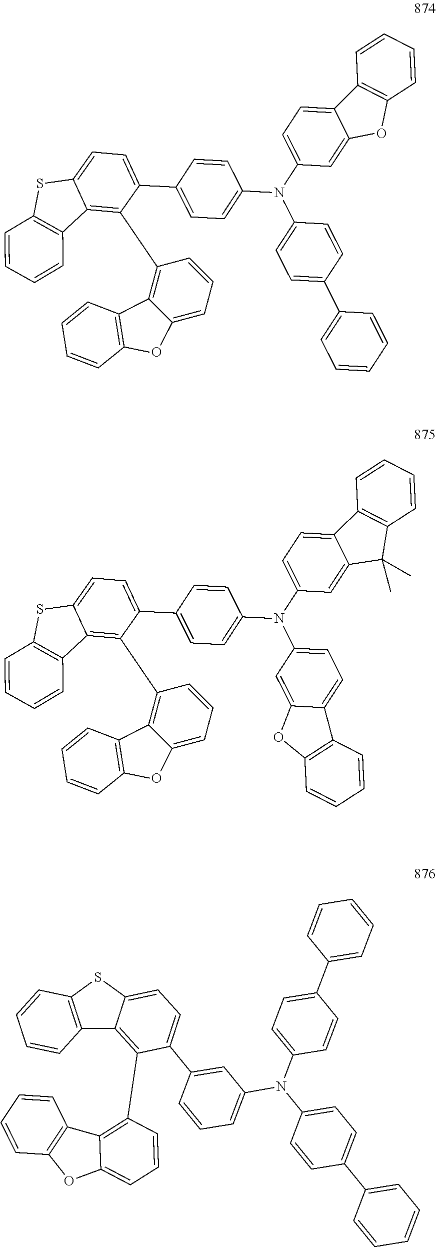 [SMILES: c1ccc(-c2ccc(N(c3ccc(-c4ccc5sc6ccccc6c5c4-c4cccc5oc6ccccc6c45)cc3)c3ccc4c(c3)oc3ccccc34)cc2)cc1 ; CC1(C)c2ccccc2-c2ccc(N(c3ccc(-c4ccc5sc6ccccc6c5c4-c4cccc5oc6ccccc6c45)cc3)c3ccc4c(c3)oc3ccccc34)cc21 ; c1ccc(-c2ccc(N(c3ccc(-c4ccccc4)cc3)c3cccc(-c4ccc5sc6ccccc6c5c4-c4cccc5oc6ccccc6c45)c3)cc2)cc1]

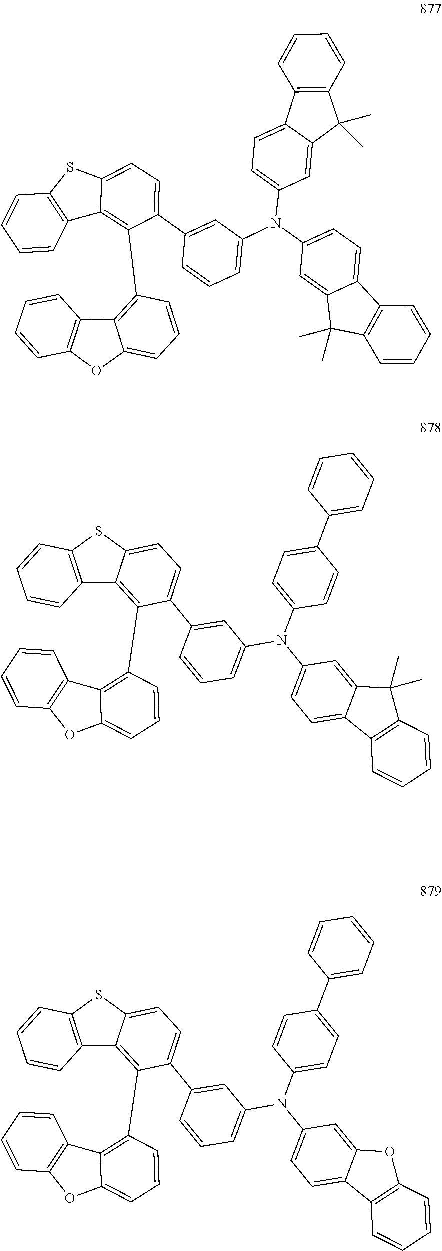 [SMILES: CC1(C)c2ccccc2-c2ccc(N(c3cccc(-c4ccc5sc6ccccc6c5c4-c4cccc5oc6ccccc6c45)c3)c3ccc4c(c3)C(C)(C)c3ccccc3-4)cc21 ; CC1(C)c2ccccc2-c2ccc(N(c3ccc(-c4ccccc4)cc3)c3cccc(-c4ccc5sc6ccccc6c5c4-c4cccc5oc6ccccc6c45)c3)cc21 ; c1ccc(-c2ccc(N(c3cccc(-c4ccc5sc6ccccc6c5c4-c4cccc5oc6ccccc6c45)c3)c3ccc4c(c3)oc3ccccc34)cc2)cc1]

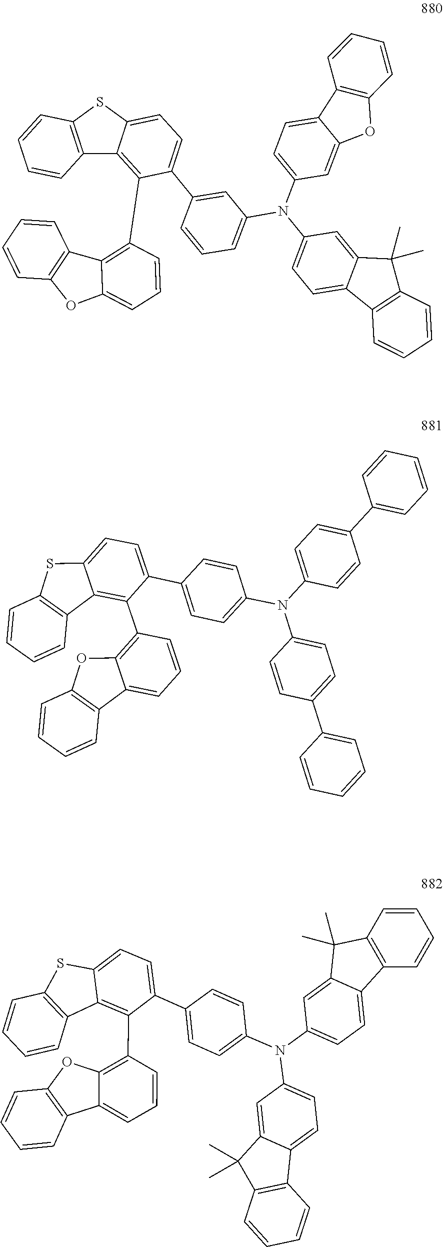 [SMILES: CC1(C)c2ccccc2-c2ccc(N(c3cccc(-c4ccc5sc6ccccc6c5c4-c4cccc5oc6ccccc6c45)c3)c3ccc4c(c3)oc3ccccc34)cc21 ; c1ccc(-c2ccc(N(c3ccc(-c4ccccc4)cc3)c3ccc(-c4ccc5sc6ccccc6c5c4-c4cccc5c4oc4ccccc45)cc3)cc2)cc1 ; CC1(C)c2ccccc2-c2ccc(N(c3ccc(-c4ccc5sc6ccccc6c5c4-c4cccc5c4oc4ccccc45)cc3)c3ccc4c(c3)C(C)(C)c3ccccc3-4)cc21]

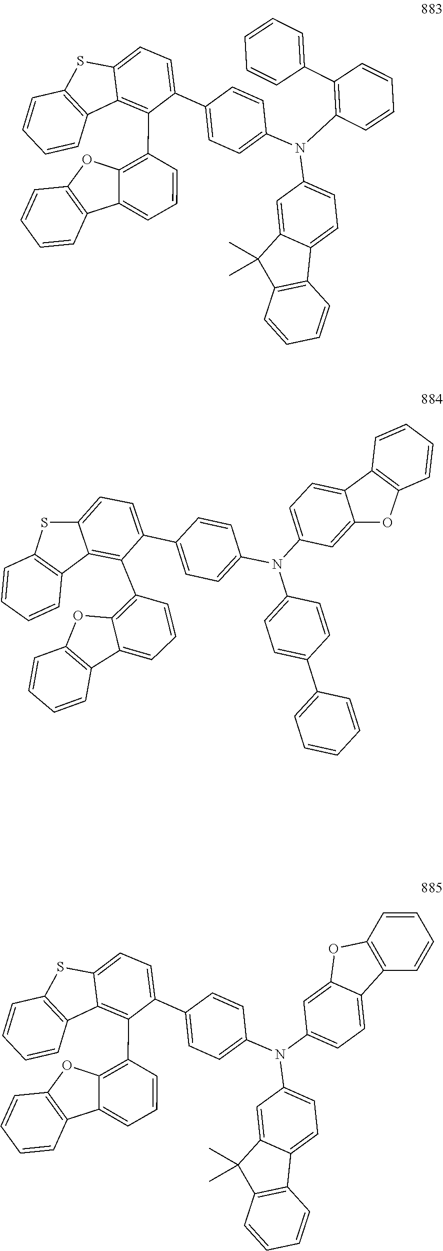 [SMILES: CC1(C)c2ccccc2-c2ccc(N(c3ccc(-c4ccc5sc6ccccc6c5c4-c4cccc5c4oc4ccccc45)cc3)c3ccccc3-c3ccccc3)cc21 ; c1ccc(-c2ccc(N(c3ccc(-c4ccc5sc6ccccc6c5c4-c4cccc5c4oc4ccccc45)cc3)c3ccc4c(c3)oc3ccccc34)cc2)cc1 ; CC1(C)c2ccccc2-c2ccc(N(c3ccc(-c4ccc5sc6ccccc6c5c4-c4cccc5c4oc4ccccc45)cc3)c3ccc4c(c3)oc3ccccc34)cc21]

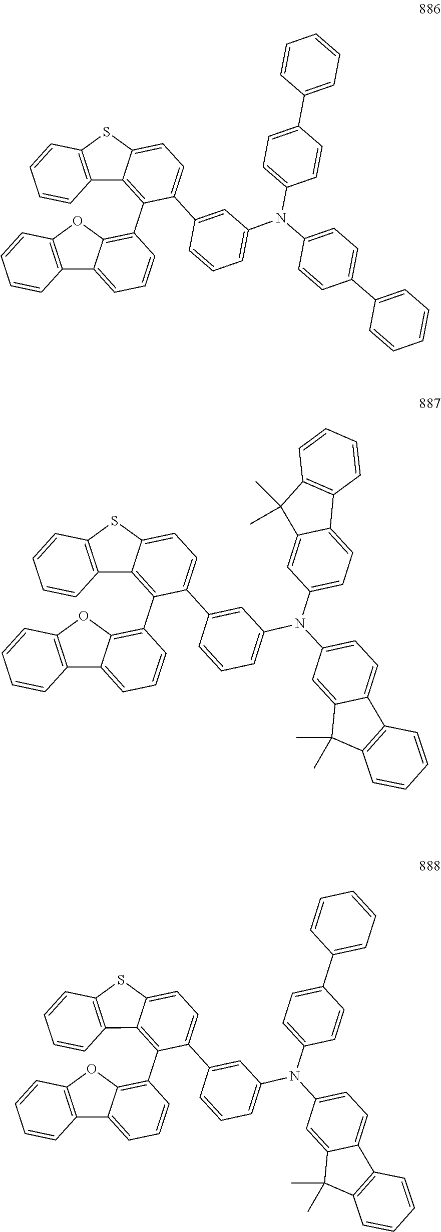 [SMILES: c1ccc(-c2ccc(N(c3ccc(-c4ccccc4)cc3)c3cccc(-c4ccc5sc6ccccc6c5c4-c4cccc5c4oc4ccccc45)c3)cc2)cc1 ; CC1(C)c2ccccc2-c2ccc(N(c3cccc(-c4ccc5sc6ccccc6c5c4-c4cccc5c4oc4ccccc45)c3)c3ccc4c(c3)C(C)(C)c3ccccc3-4)cc21 ; CC1(C)c2ccccc2-c2ccc(N(c3ccc(-c4ccccc4)cc3)c3cccc(-c4ccc5sc6ccccc6c5c4-c4cccc5c4oc4ccccc45)c3)cc21]

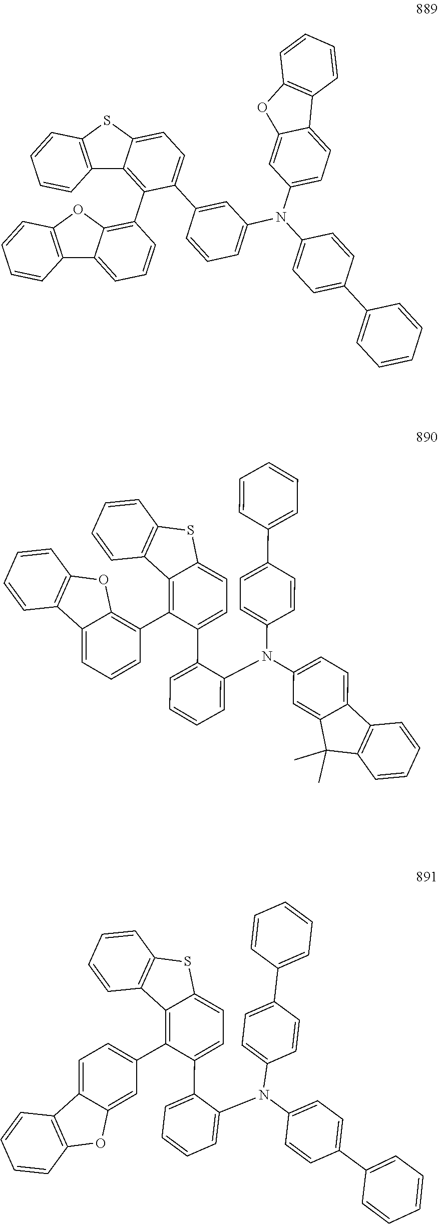 [SMILES: c1ccc(-c2ccc(N(c3cccc(-c4ccc5sc6ccccc6c5c4-c4cccc5c4oc4ccccc45)c3)c3ccc4c(c3)oc3ccccc34)cc2)cc1 ; CC1(C)c2ccccc2-c2ccc(N(c3ccc(-c4ccccc4)cc3)c3ccccc3-c3ccc4sc5ccccc5c4c3-c3cccc4c3oc3ccccc34)cc21 ; c1ccc(-c2ccc(N(c3ccc(-c4ccccc4)cc3)c3ccccc3-c3ccc4sc5ccccc5c4c3-c3ccc4c(c3)oc3ccccc34)cc2)cc1]

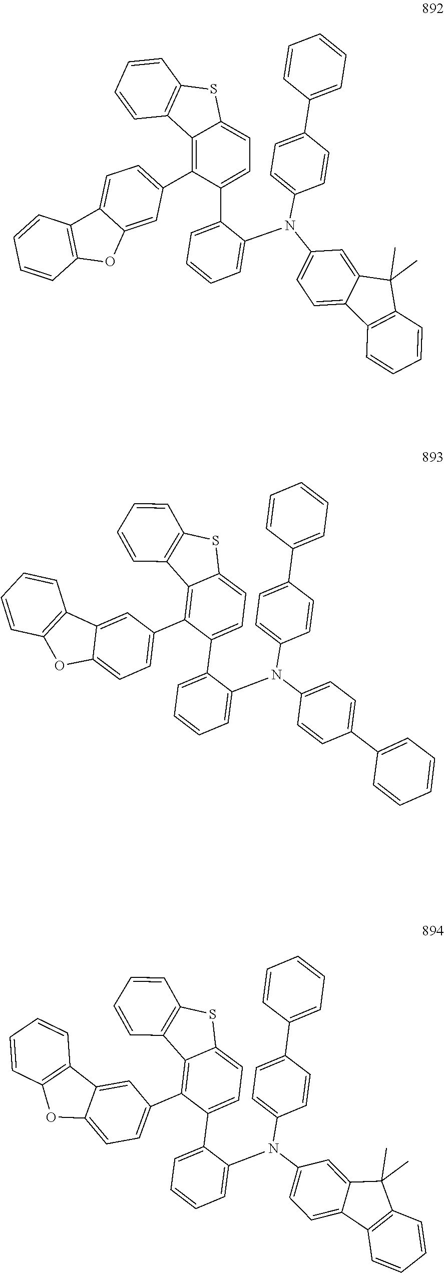 [SMILES: CC1(C)c2ccccc2-c2ccc(N(c3ccc(-c4ccccc4)cc3)c3ccccc3-c3ccc4sc5ccccc5c4c3-c3ccc4c(c3)oc3ccccc34)cc21 ; c1ccc(-c2ccc(N(c3ccc(-c4ccccc4)cc3)c3ccccc3-c3ccc4sc5ccccc5c4c3-c3ccc4oc5ccccc5c4c3)cc2)cc1 ; CC1(C)c2ccccc2-c2ccc(N(c3ccc(-c4ccccc4)cc3)c3ccccc3-c3ccc4sc5ccccc5c4c3-c3ccc4oc5ccccc5c4c3)cc21]

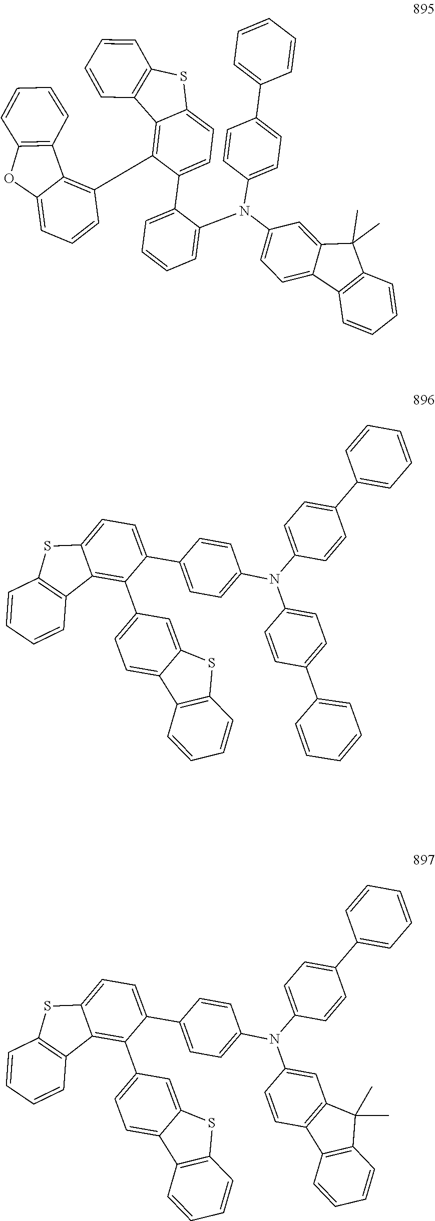 [SMILES: CC1(C)c2ccccc2-c2ccc(N(c3ccc(-c4ccccc4)cc3)c3ccccc3-c3ccc4sc5ccccc5c4c3-c3cccc4oc5ccccc5c34)cc21 ; c1ccc(-c2ccc(N(c3ccc(-c4ccccc4)cc3)c3ccc(-c4ccc5sc6ccccc6c5c4-c4ccc5c(c4)sc4ccccc45)cc3)cc2)cc1 ; CC1(C)c2ccccc2-c2ccc(N(c3ccc(-c4ccccc4)cc3)c3ccc(-c4ccc5sc6ccccc6c5c4-c4ccc5c(c4)sc4ccccc45)cc3)cc21]

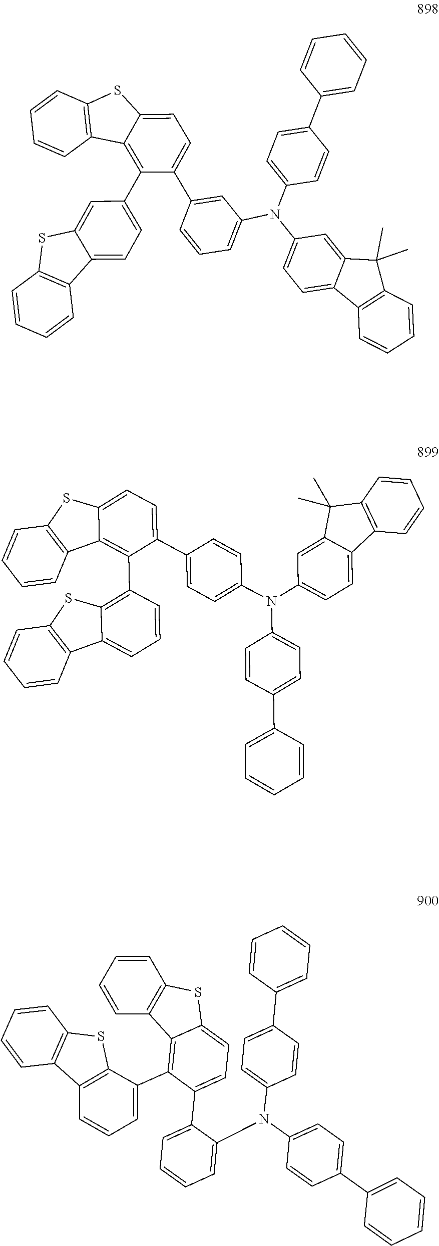 [SMILES: CC1(C)c2ccccc2-c2ccc(N(c3ccc(-c4ccccc4)cc3)c3cccc(-c4ccc5sc6ccccc6c5c4-c4ccc5c(c4)sc4ccccc45)c3)cc21 ; CC1(C)c2ccccc2-c2ccc(N(c3ccc(-c4ccccc4)cc3)c3ccc(-c4ccc5sc6ccccc6c5c4-c4cccc5c4sc4ccccc45)cc3)cc21 ; c1ccc(-c2ccc(N(c3ccc(-c4ccccc4)cc3)c3ccccc3-c3ccc4sc5ccccc5c4c3-c3cccc4c3sc3ccccc34)cc2)cc1]

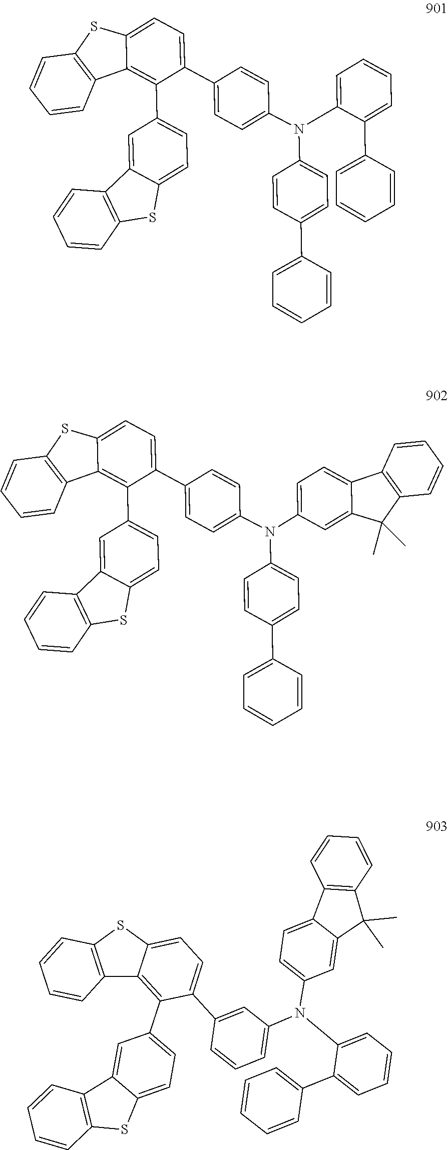 [SMILES: c1ccc(-c2ccc(N(c3ccc(-c4ccc5sc6ccccc6c5c4-c4ccc5sc6ccccc6c5c4)cc3)c3ccccc3-c3ccccc3)cc2)cc1 ; CC1(C)c2ccccc2-c2ccc(N(c3ccc(-c4ccccc4)cc3)c3ccc(-c4ccc5sc6ccccc6c5c4-c4ccc5sc6ccccc6c5c4)cc3)cc21 ; CC1(C)c2ccccc2-c2ccc(N(c3cccc(-c4ccc5sc6ccccc6c5c4-c4ccc5sc6ccccc6c5c4)c3)c3ccccc3-c3ccccc3)cc21]

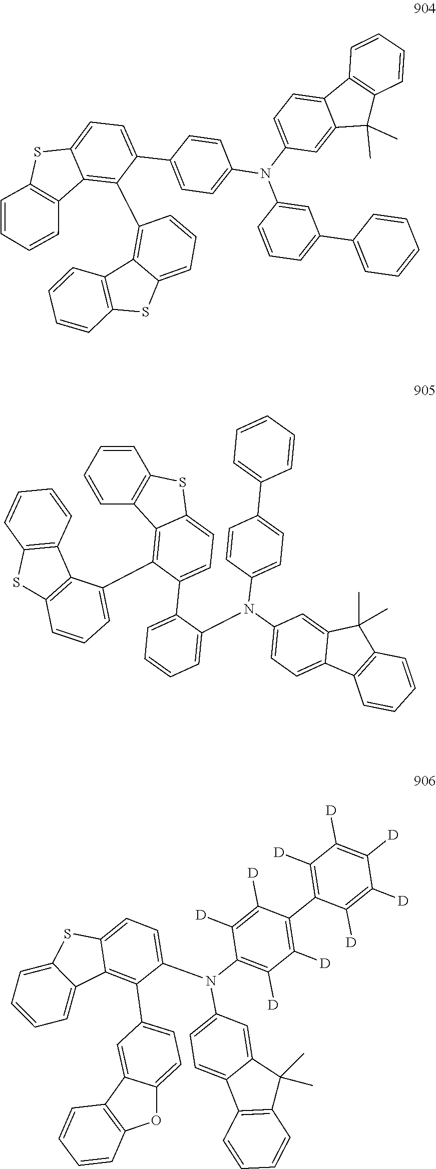 [SMILES: CC1(C)c2ccccc2-c2ccc(N(c3ccc(-c4ccc5sc6ccccc6c5c4-c4cccc5sc6ccccc6c45)cc3)c3cccc(-c4ccccc4)c3)cc21 ; CC1(C)c2ccccc2-c2ccc(N(c3ccc(-c4ccccc4)cc3)c3ccccc3-c3ccc4sc5ccccc5c4c3-c3cccc4sc5ccccc5c34)cc21 ; [2H]c1c([2H])c([2H])c(-c2c([2H])c([2H])c(N(c3ccc4c(c3)C(C)(C)c3ccccc3-4)c3ccc4sc5ccccc5c4c3-c3ccc4oc5ccccc5c4c3)c([2H])c2[2H])c([2H])c1[2H]]

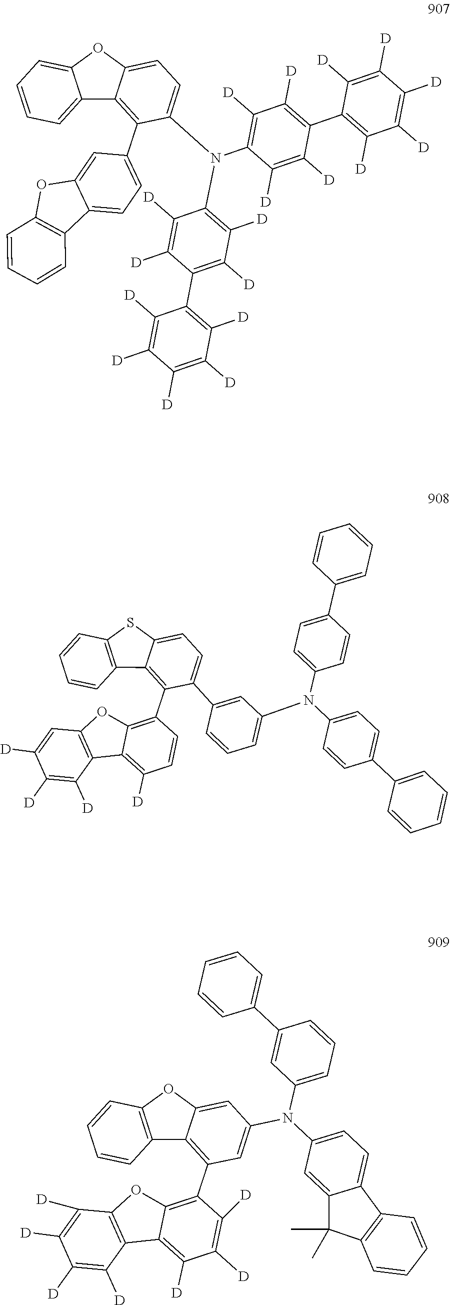 [SMILES: [2H]c1c([2H])c([2H])c(-c2c([2H])c([2H])c(N(c3ccc4oc5ccccc5c4c3-c3ccc4c(c3)oc3ccccc34)c3c([2H])c([2H])c(-c4c([2H])c([2H])c([2H])c([2H])c4[2H])c([2H])c3[2H])c([2H])c2[2H])c([2H])c1[2H] ; [2H]c1cc2oc3c(-c4c(-c5cccc(N(c6ccc(-c7ccccc7)cc6)c6ccc(-c7ccccc7)cc6)c5)ccc5sc6ccccc6c45)ccc([2H])c3c2c([2H])c1[2H] ; [2H]c1c([2H])c([2H])c2c(oc3c(-c4cc(N(c5cccc(-c6ccccc6)c5)c5ccc6c(c5)C(C)(C)c5ccccc5-6)cc5oc6ccccc6c45)c([2H])c([2H])c([2H])c32)c1[2H]]

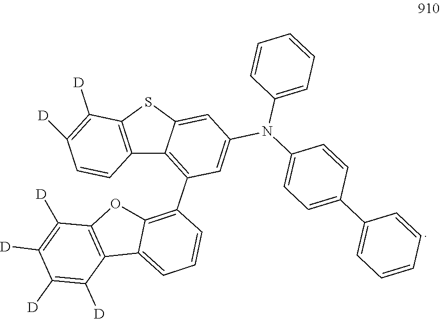 [SMILES: [2H]c1ccc2c(sc3cc(N(c4ccccc4)c4ccc(-c5ccccc5)cc4)cc(-c4cccc5c4oc4c([2H])c([2H])c([2H])c([2H])c45)c32)c1[2H]]

In a second aspect, the present disclosure provides an organic electroluminescent device comprising an anode and a cathode, and a functional layer disposed between the anode and the cathode; wherein the functional layer comprises the arylamine compound described in the first aspect of the present disclosure.

The arylamine compound provided in the present disclosure can be used to form at least one organic film layer in the functional layer, to improve characteristics such as luminous efficiency and lifetime of the organic electroluminescent device.

Optionally, the functional layer comprises a hole auxiliary layer, which contains the arylamine compound. Among them, the hole auxiliary layer can be composed of the arylamine compound provided in the present disclosure or can be composed of the arylamine compound provided in the present disclosure and other materials collectively.

Optionally, the functional layer further comprises a hole transport area, which comprises a hole transport layer (also referred to a first hole transport layer) and a hole auxiliary layer (also referred to a second hole transport layer or a hole adjustment layer). The hole transport layer is located between the anode and the organic light-emitting layer, and the hole adjustment layer is located between the hole transport layer and the organic light-emitting layer. In some embodiments, the hole adjustment layer is composed of the arylamine compound provided in the present disclosure or is composed of the arylamine compound provided in the present disclosure and other materials collectively.

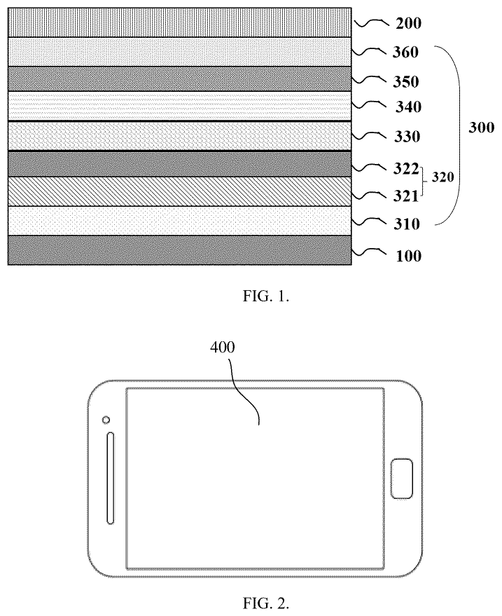

According to a specific embodiment, the organic electroluminescent device as shown in FIG. 1, includes an anode 100, a hole injection layer 310, a hole transport layer 321, a hole auxiliary layer 322, an organic light-emitting layer 330, a hole blocking layer 340, an electron transport layer 350, an electron injection layer 360, and a cathode 200 sequentially stacked.

In the present disclosure, the anode 100 comprises an anode material, which is preferably a high-work function material contributing to the injection of holes into the functional layer. Specific examples of the anode material include: metals such as nickel, platinum, vanadium, chromium, copper, zinc and gold, or alloys thereof; metal oxides such as zinc oxide, indium oxide, indium tin oxide (ITO), and indium zinc oxide (IZO); combinations of metals and oxides, such as ZnO:Al or SnO₂:Sb; or conductive polymers such as poly(3-methylthiophene), poly[3,4-(ethylene-1,2-dioxy)thiophene](PEDT), polypyrrole, and polyaniline, but are not limited thereto. Preferably, a transparent electrode comprising indium tin oxide (ITO) as the anode is included.

In the present disclosure, the hole transport layer and the hole adjustment layer may include one or more hole transport material(s), respectively. The hole transport materials may be selected from a carbazole multimer, a carbazole-linked triarylamine compound, and other types of compounds, and specifically may be selected from the compounds as shown below and any combination thereof:

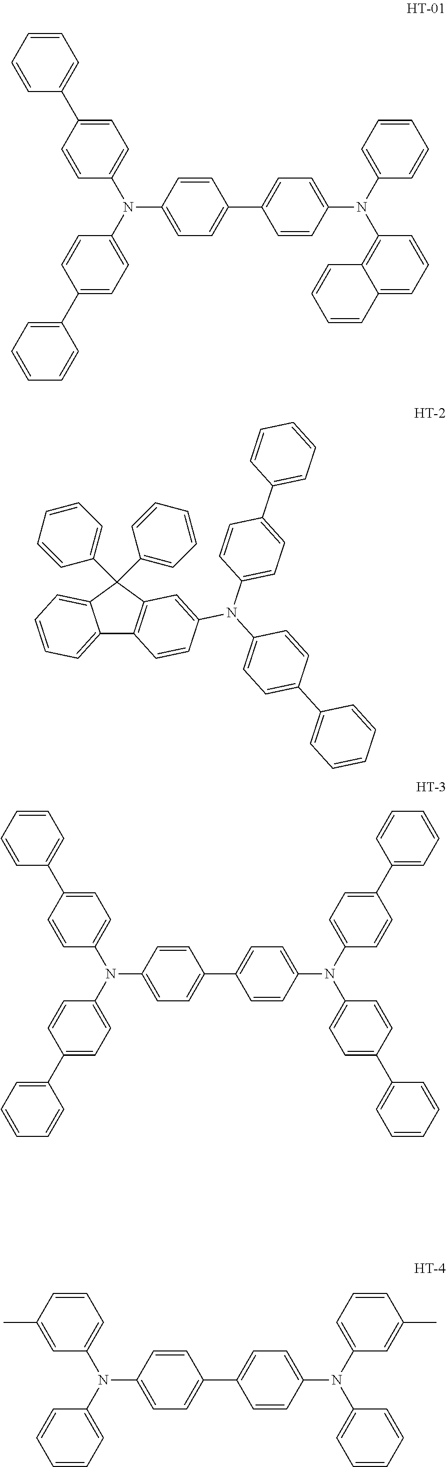

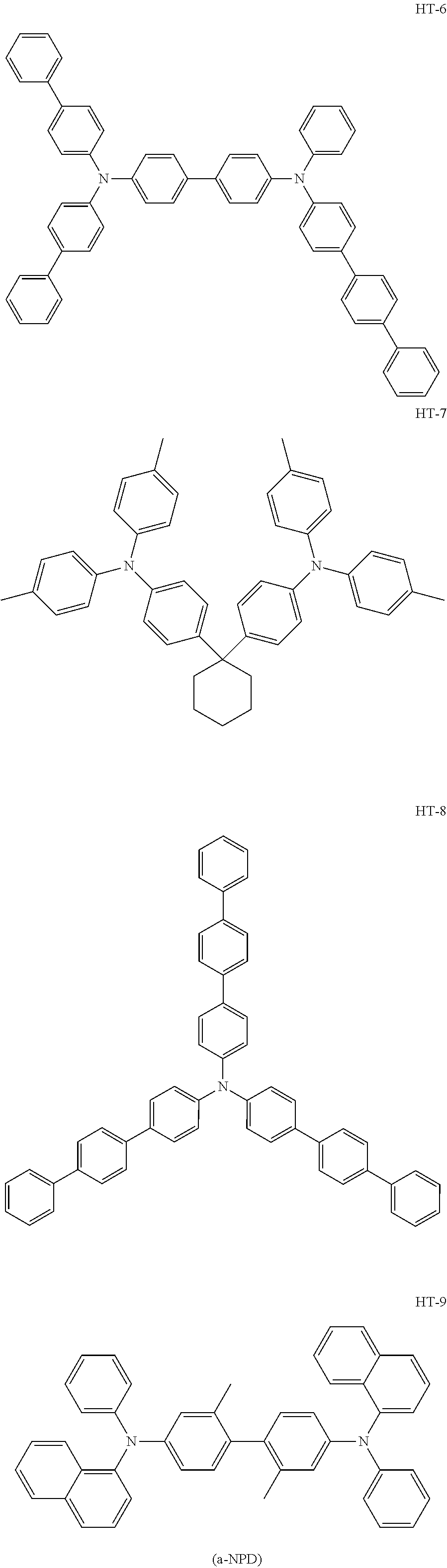

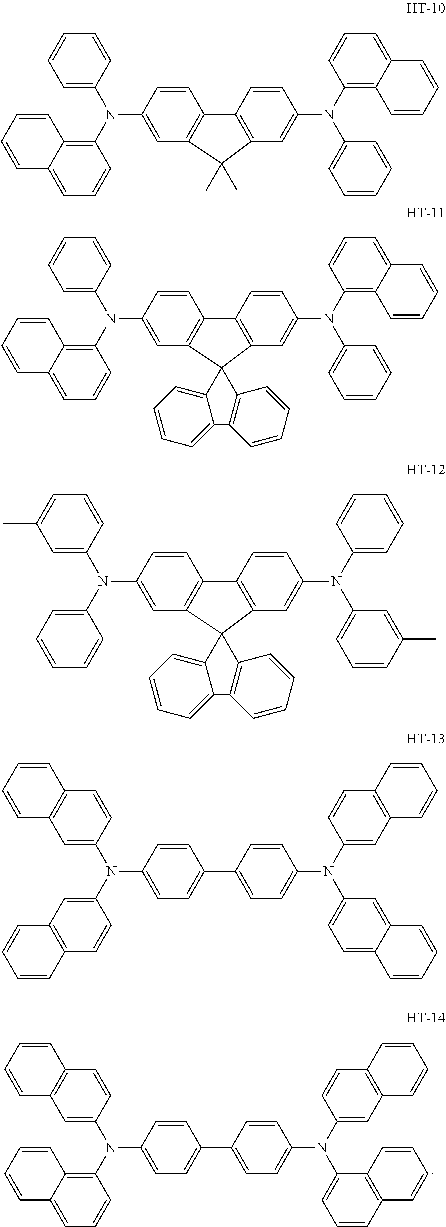

In an embodiment, the hole transport layer 321 is composed of HT-01.

In an embodiment, the hole transport layer 322 is composed of the compounds of the present disclosure.

Optionally, a hole injection layer 310 may be further disposed between the anode 100 and the hole transport layer 321 to enhance the ability to inject holes into the hole transport layer 321. The hole injection layer 310 may choose to use a benzidine derivative, a starburst arylamine-based compound, a phthalocyanine derivative, and other materials, and it is not particularly defined in the present disclosure. For example, the material of the hole injection layer 310 is selected from the compounds as shown below and any combination thereof:

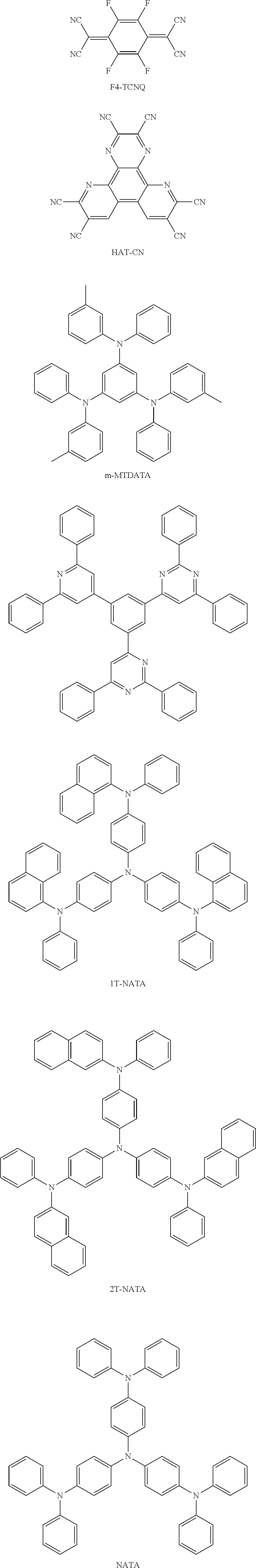

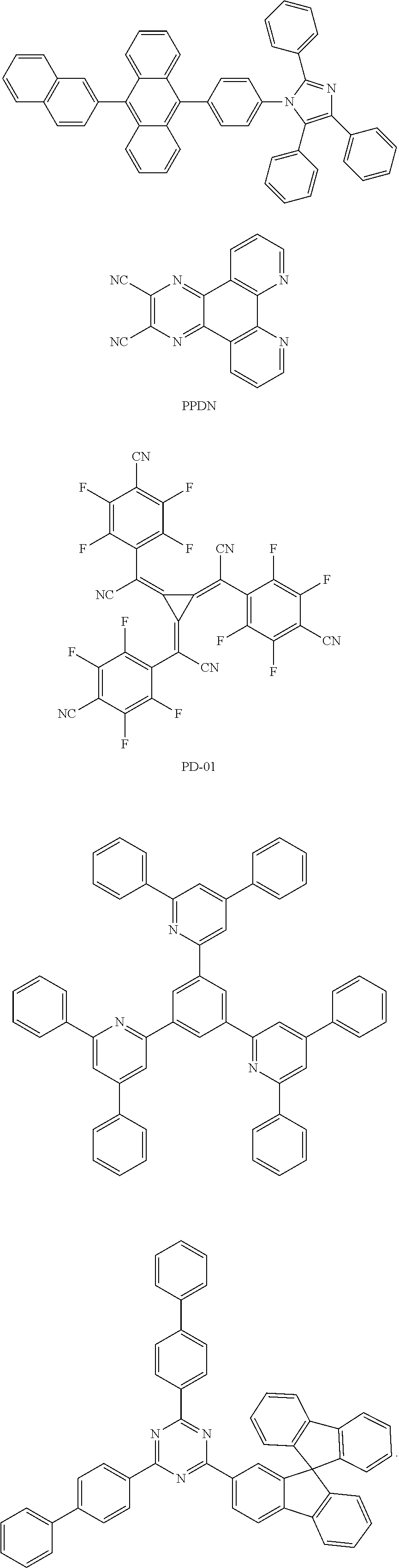

In an embodiment of the present disclosure, the hole injection layer 310 is composed of PD-01 and HT-01.

Optionally, the organic light-emitting layer 330 may be composed of a single luminescent material or may comprise a host material and a guest material. Optionally, the organic light-emitting layer 330 is composed of a host material and a guest material. The holes injected into the organic light-emitting layer 330 and the electrons injected into the organic light-emitting layer 330 can recombine in the organic light-emitting layer 330 to form excitons. The excitons transmit energy to the host material, and the host material transmits the energy to the guest material, thereby enabling the guest material to emit light.

The host material of the organic light-emitting layer 330 may be a metal chelating compound, a stilbene-based derivative, an arylamine derivative, a dibenzofuran derivative, or other types of material. The host material of the organic light-emitting layer 330 may be one compound or a combination of two or more compounds. Optionally, the host material includes the arylamine compound of the present disclosure.

The guest material of the organic light-emitting layer 330 may be a compound with a condensed aryl ring or a derivative thereof, a compound with a heteroaryl ring or a derivative thereof, an arylamine derivative, or other materials, and it is not particularly limited in the present disclosure. The guest material is also known as a doping material or a dopant. The guest material can be classified into a fluorescent dopant and a phosphorescent dopant depending on the type of luminescence. The specific examples of the phosphorescent dopant include but are not limited to,

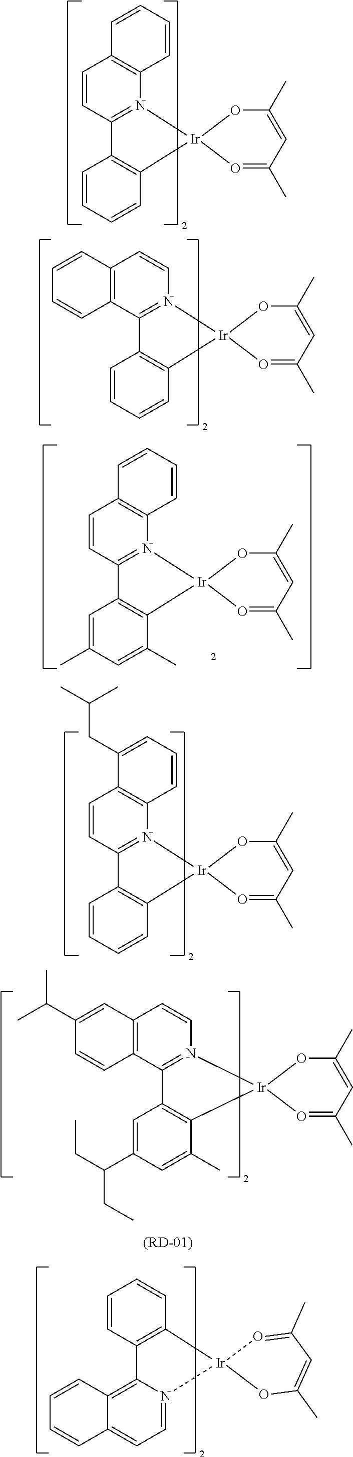

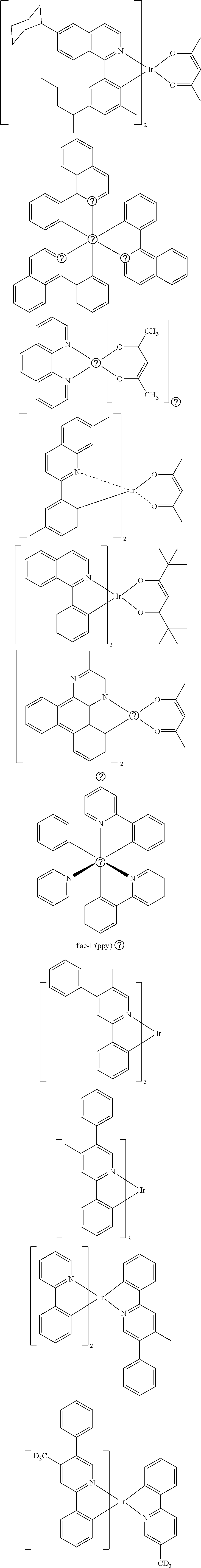

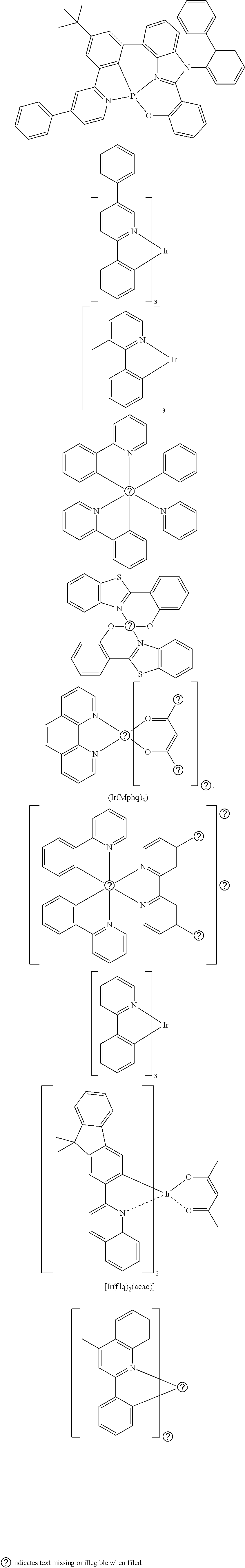

In an embodiment of the present disclosure, the organic electroluminescent device is a red organic electroluminescent device. In a more specific embodiment, the host material of the organic light-emitting layer 330 comprises RH-01

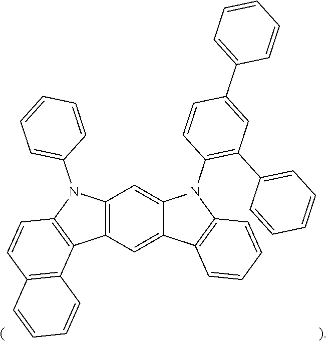 [SMILES: c1ccc(-c2ccc(-n3c4ccccc4c4cc5c6c7ccccc7ccc6n(-c6ccccc6)c5cc43)c(-c3ccccc3)c2)cc1]

The guest material may be, for example, RD-01.

The hole blocking layer 340 may be a single-layer structure or a multi-layer structure, which may comprise one or more hole blocking material(s).

In a more specific embodiment, the material of the hole blocking layer 340 is HB-01

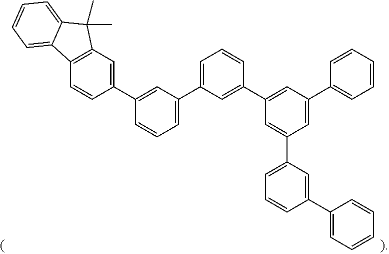 [SMILES: CC1(C)c2ccccc2-c2ccc(-c3cccc(-c4cccc(-c5cc(-c6ccccc6)cc(-c6cccc(-c7ccccc7)c6)c5)c4)c3)cc21]

The electron transport layer 350 may be a single-layer structure or a multi-layer structure, which may comprise one or more electron transport material(s). The electron transport materials may be selected from, but not limited to LiQ, a benzimidazole derivative, an oxadiazole derivative, a quinoxaline derivative, and other electron transport materials, and it is not particularly limited in the present disclosure. The material of the electron transport layer 350 includes, but is not limited to the following compounds:

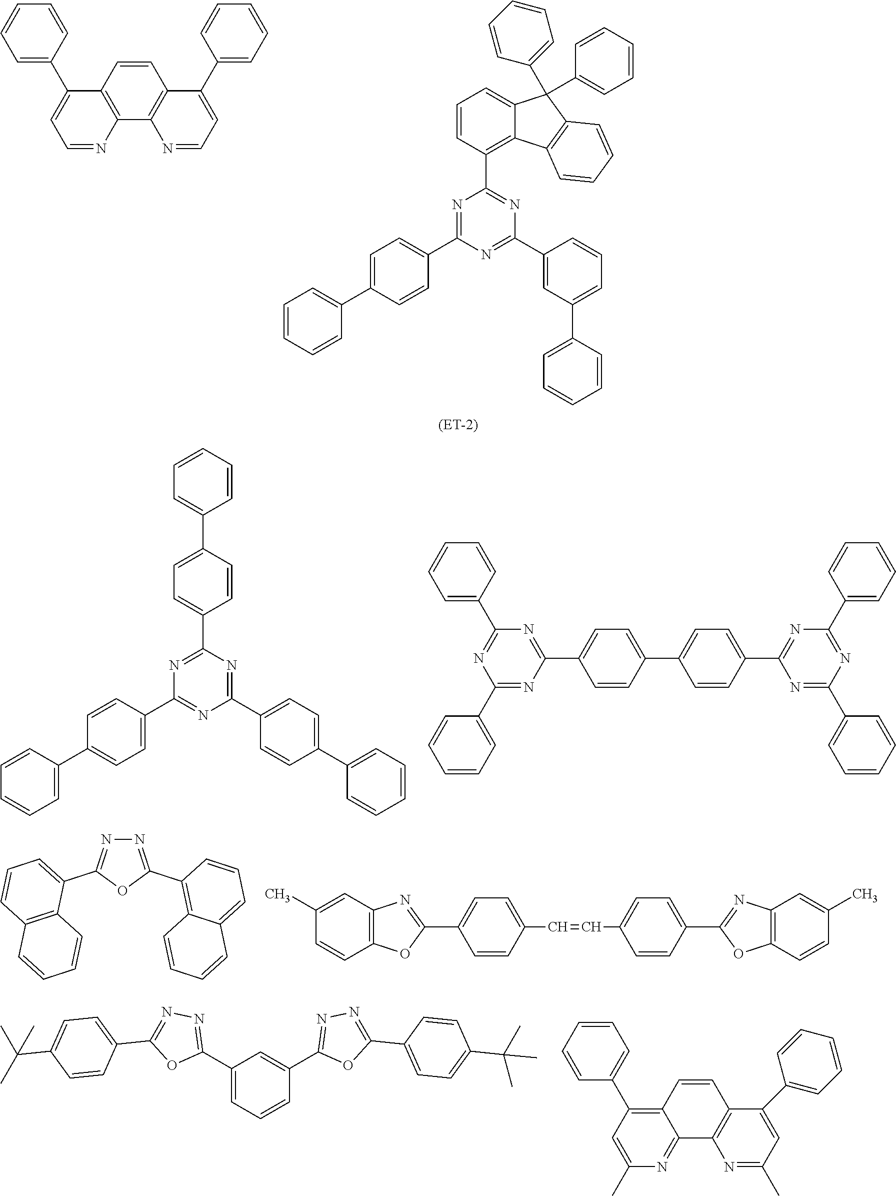

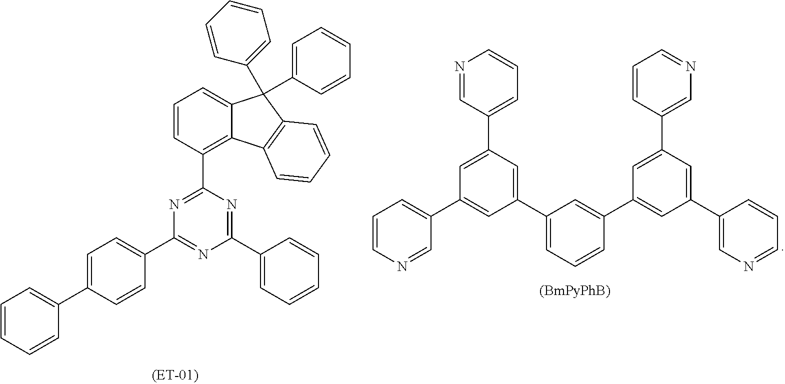 [SMILES: c1ccc(-c2ccc(-c3nc(-c4ccccc4)nc(-c4cccc5c4-c4ccccc4C5(c4ccccc4)c4ccccc4)n3)cc2)cc1 ; c1cncc(-c2cc(-c3cccnc3)cc(-c3cccc(-c4cc(-c5cccnc5)cc(-c5cccnc5)c4)c3)c2)c1]

In an embodiment of the present disclosure, the electron transport layer 350 is composed of ET-01 and LiQ.

In an embodiment, the cathode 200 comprises a cathode material, which is a low-work function material contributing to the injection of electrons into the functional layer. Specific examples of the cathode material include, but are not limited to, metals such as magnesium, calcium, sodium, potassium, titanium, indium, yttrium, lithium, gadolinium, aluminum, silver, tin, and lead, or alloys thereof; or multilayer materials such as LiF/Al, Liq/Al, LiO₂/Al, LiF/Ca, LiF/Al, and BaF₂/Ca. Optionally, a metal electrode comprising magnesium and silver as the cathode is included.

Optionally, an electron injection layer 360 may be further provided between the cathode 200 and the electron transport layer 350 to enhance the ability to inject electrons into the electron transport layer 350. The electron injection layer 360 may comprise an inorganic material such as an alkali metal sulfide and an alkali metal halide or may comprise a complex of an alkali metal and an organic compound. In an embodiment of the present disclosure, the electron injection layer 360 comprises ytterbium (Yb).

In a third aspect, the present disclosure provides an electronic apparatus, comprising the organic electroluminescent device described in the second aspect.

According to an embodiment, the electronic apparatus as shown in FIG. 2 is an electronic apparatus 400 comprising the above-described organic electroluminescent device. The electronic apparatus 400 may be, for example, a display apparatus, a lighting apparatus, an optical communication apparatus, or other types of electronic apparatus, the examples of which may include, but be not limited to, a computer screen, a mobile phone screen, a television, an electronic paper, an emergency lamp, an optical module, etc.

The synthesis method of the arylamine compounds of the present disclosure is described below in conjunction with synthesis examples. However, the present disclosure is not limited by these examples as a result.

### SYNTHESIS EXAMPLES

Professionals in the field to which they pertain should realize that the chemical reactions described in the present disclosure can be used to properly prepare many arylamine compounds in the present disclosure, and other methods for preparing the compounds in the present disclosure are considered to be within the scope of this present disclosure. For example, according to the present disclosure, the synthesis of those non-exemplified compounds can be successfully completed by those skilled in the field through modification methods, such as appropriate protection of interfering groups, by using other known reagents in addition to the ones described in the present disclosure, or by making some conventional modifications to the reaction conditions. The compounds of which the synthetic methods not mentioned in the present disclosure are all raw material products which are commerically available.

### 1. Synthesis of Intermediate IM-A1

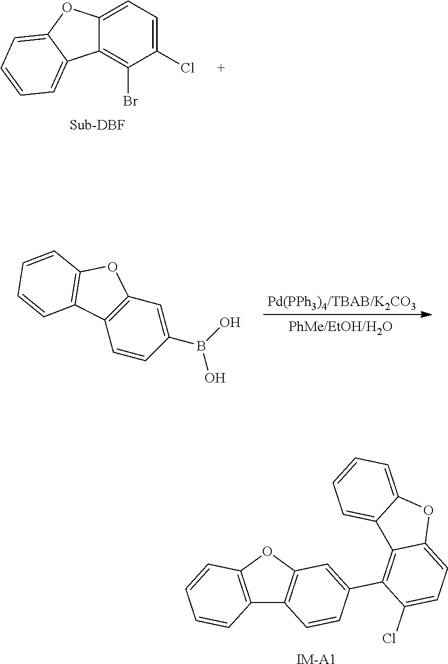

Under nitrogen protection, to a reaction flask was added Sub-DBF (60.0 g, 213.12 mmol), dibenzofuran-3-boronic acid (45.2 g, 213.12 mmol), potassium carbonate (64.8 g, 468.87 mmol), toluene (500 mL), ethanol (250 mL) and water (150 mL). The resulting mixture was stirred and heated to 50° C. to 60° C., followed by addition of tetrakis(triphenylphosphine) palladium (4.92 g, 4.26 mmol) and tetrabutylammonium bromide (TBAB) (13.8 g, 42.62 mmol) rapidly, and then was heated to 70° C. to 75° C. and refluxed for 12 hours. As the reaction was completed, the resulting solution was allowed to cool and then was extracted with dichloromethane. After separation, the organic phase was washed with water until neutral, the organic phase was dried, filtrated, and the filtrate was concentrated in vacuum to obtain a crude product. The crude product was recrystallized with a mixed solvent of ethyl acetate and petroleum ether to LC>98%, and then was dried, to give a white solid IM-A1 (55.65 g, yield 70.8%).

### 2. Synthesis of Intermediate IM-A2

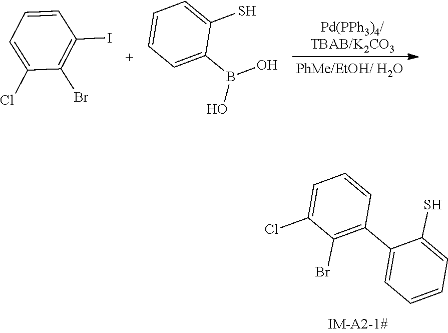

(1) Under nitrogen protection, to a reaction flask was added 2-bromo-1-chloro-3-iodobenzene (80.0 g, 252.09 mmol), o-mercaptophenylboronic acid (38.8 g, 252.09 mmol), potassium carbonate (76.64 g, 554.59 mmol), toluene (650 mL), ethanol (300 mL) and water (150 mL). The resulting mixture was stirred and heated to 50° C. to 60° C., followed by addition of tetrakis(triphenylphosphine) palladium (5.82 g, 5.04 mmol) and tetrabutylammonium bromide (TBAB) (16.25 g, 50.42 mmol) rapidly, and then was heated to 70° C. to 75° C. and refluxed for 12 hours. As the reaction was completed, the resulting solution was allowed to cool and then was extracted with dichloromethane. After separation, the organic phase was washed with water until neutral, followed by being dried, filtered and the filtrate was concentrated in vacuum to yield a crude product. The crude product was recrystallized with a mixed solvent of ethyl acetate and petroleum ether (volume ratio of 1:3) to LC>98%, and then was dried, to give a white solid IM-A2-1 #(49.24 g, yield 65.2%).

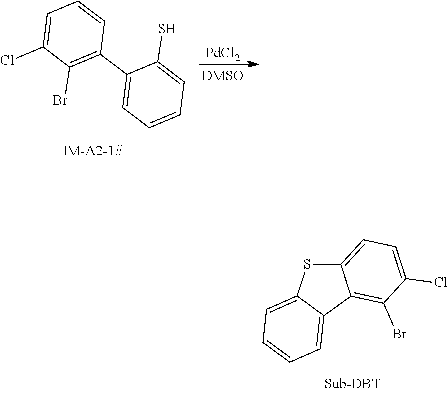

(2) Under nitrogen protection, to a reaction flask was added IM-A2-1 #(48.6 g, 162.21 mmol), palladium dichloride (1.44 g, 8.11 mmol) and dimethyl sulfoxide (400 mL). The resulting mixture was stirred and heated to 140° C. and reacted for 12 hours. The reaction was quenched, and the reaction solution was allowed to cool to 80° C. and was poured into 800 mL of deionized water, followed by being stirred for 15 minutes, and held for 1 hour to precipitate a large amount of solid out. The precipitate was filtered, and the filter cake was rinsed with 300 mL of deionized water for 3 times. The resulting crude product was recrystallized with a mixed solvent of ethyl acetate and n-heptane to yield a white solid Sub-DBT (42.2 g, yield 87.5%).

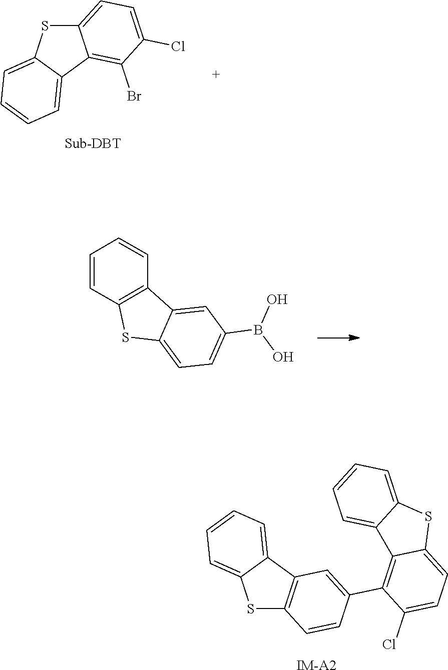

(3) Intermediate IM-A2 was synthesized using the same method as the intermediate IM-A1, except that Sub-DBT was used instead of Sub-DBF, and dibenzothiophene-2-boronic acid was used instead of dibenzofuran-3-boronic acid. All other conditions remained unchanged, yielding intermediate IM-A2 (25.4 g, 69.6% yield).

### 3. Synthesis of Intermediates IM-A3 to IM-A17

Intermediates IM-A3 to IM-A17 listed in Table 1 were synthesized following step (3) of the preparation method of IM-A2, except that raw material 1 was used instead of Sub-DBT, and raw material 2 was used instead of dibenzothiophene-2-boronic acid. The main raw materials used, the synthesized intermediates, and their yields are shown in Table 1.

TABLE 1

| Intermediate No. | Raw Material 1 | Raw Material 2 | Product | Yield/% |
|---|---|---|---|---|
| IM-A3 | 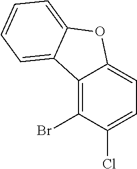 [SMILES: Clc1ccc2oc3ccccc3c2c1Br] | 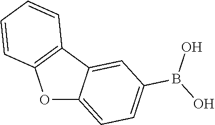 [SMILES: OB(O)c1ccc2oc3ccccc3c2c1] | 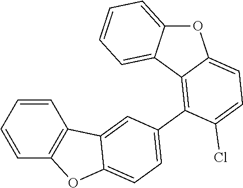 [SMILES: Clc1ccc2oc3ccccc3c2c1-c1ccc2oc3ccccc3c2c1] | 68.3 |
| | Sub-DBF | | | |
| IM-A4 | 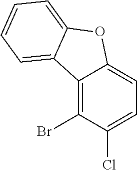 [SMILES: Clc1ccc2oc3ccccc3c2c1Br] | 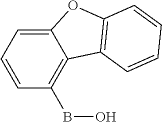 | 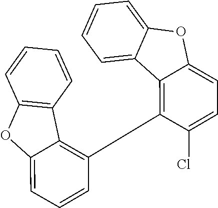 [SMILES: Clc1ccc2oc3ccccc3c2c1-c1cccc2oc3ccccc3c12] | 62.2 |
| | Sub-DBF | | | |
| IM-A5 | 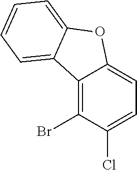 [SMILES: Clc1ccc2oc3ccccc3c2c1Br] | 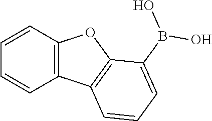 [SMILES: OB(O)c1cccc2c1oc1ccccc12] | 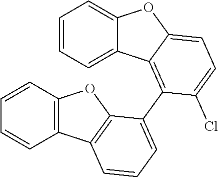 [SMILES: Clc1ccc2oc3ccccc3c2c1-c1cccc2c1oc1ccccc12] | 62.6 |
| | Sub-DBF | | | |
| IM-A6 | 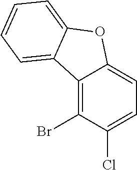 [SMILES: Clc1ccc2oc3ccccc3c2c1Br] | 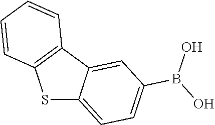 [SMILES: OB(O)c1ccc2sc3ccccc3c2c1] | 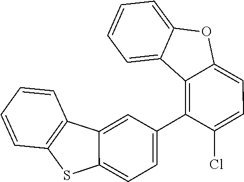 [SMILES: Clc1ccc2oc3ccccc3c2c1-c1ccc2sc3ccccc3c2c1] | 68.1 |
| | Sub-DB | | | |
| IM-A7 | 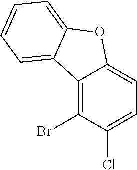 [SMILES: Clc1ccc2oc3ccccc3c2c1Br] | 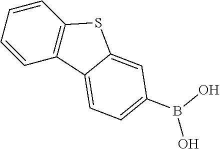 [SMILES: OB(O)c1ccc2c(c1)sc1ccccc12] | 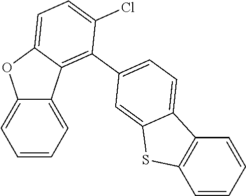 [SMILES: Clc1ccc2oc3ccccc3c2c1-c1ccc2c(c1)sc1ccccc12] | 71.2 |
| | Sub-DB | | | |
| IM-A8 | 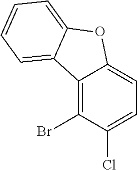 [SMILES: Clc1ccc2oc3ccccc3c2c1Br] | 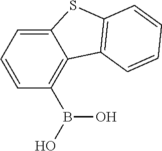 [SMILES: OB(O)c1cccc2sc3ccccc3c12] | 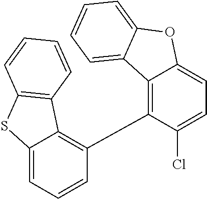 [SMILES: Clc1ccc2oc3ccccc3c2c1-c1cccc2sc3ccccc3c12] | 63.1 |
| | Sub-DB | | | |
| IM-A9 | 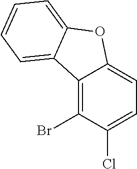 [SMILES: Clc1ccc2oc3ccccc3c2c1Br] | 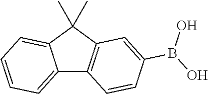 [SMILES: CC1(C)c2ccccc2-c2ccc(B(O)O)cc21] | 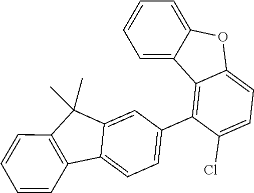 [SMILES: CC1(C)c2ccccc2-c2ccc(-c3c(Cl)ccc4oc5ccccc5c34)cc21] | 60.2 |
| | Sub-DB | | | |
| IM-A10 | 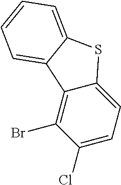 [SMILES: Clc1ccc2sc3ccccc3c2c1Br] | 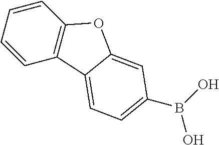 [SMILES: OB(O)c1ccc2c(c1)oc1ccccc12] | 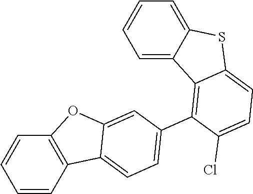 [SMILES: Clc1ccc2sc3ccccc3c2c1-c1ccc2c(c1)oc1ccccc12] | 71.6 |
| | Sub-DBT | | | |
| IM-A11 | 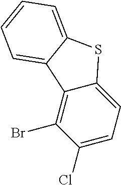 [SMILES: Clc1ccc2sc3ccccc3c2c1Br] | 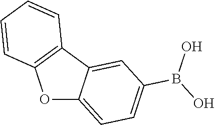 [SMILES: OB(O)c1ccc2oc3ccccc3c2c1] | 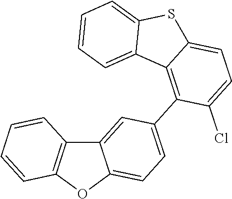 [SMILES: Clc1ccc2sc3ccccc3c2c1-c1ccc2oc3ccccc3c2c1] | 70.5 |
| | Sub-DBT | | | |
| IM-A12 | 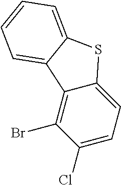 [SMILES: Clc1ccc2sc3ccccc3c2c1Br] | 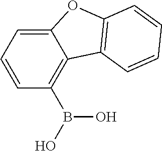 [SMILES: OB(O)c1cccc2oc3ccccc3c12] | 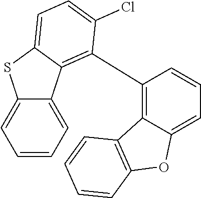 [SMILES: Clc1ccc2sc3ccccc3c2c1-c1cccc2oc3ccccc3c12] | 62.6 |
| | Sub-DBT | | | |
| IM-A13 | 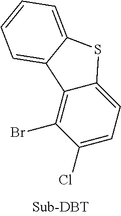 [SMILES: Clc1ccc2sc3ccccc3c2c1Br] | 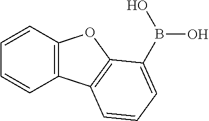 [SMILES: OB(O)c1cccc2c1oc1ccccc12] | 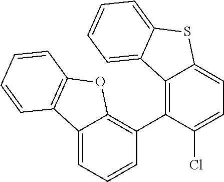 [SMILES: Clc1ccc2sc3ccccc3c2c1-c1cccc2c1oc1ccccc12] | 63.9 |
| IM-A14 | 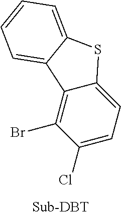 [SMILES: Clc1ccc2sc3ccccc3c2c1Br] | 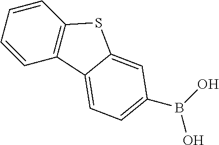 [SMILES: OB(O)c1ccc2c(c1)sc1ccccc12] | 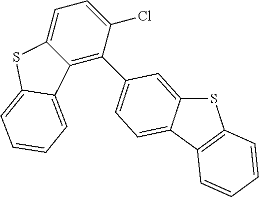 [SMILES: Clc1ccc2sc3ccccc3c2c1-c1ccc2c(c1)sc1ccccc12] | 69.7 |
| IM-A15 | 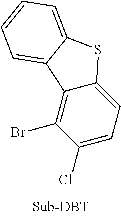 [SMILES: Clc1ccc2sc3ccccc3c2c1Br] | 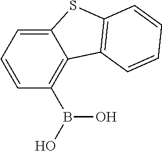 [SMILES: OB(O)c1cccc2sc3ccccc3c12] | 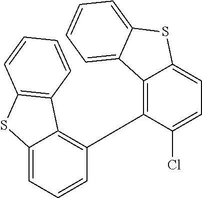 [SMILES: Clc1ccc2sc3ccccc3c2c1-c1cccc2sc3ccccc3c12] | 64.1 |
| IM-A16 | 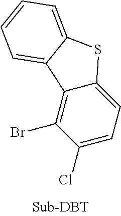 [SMILES: Clc1ccc2sc3ccccc3c2c1Br] | 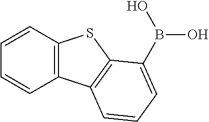 [SMILES: OB(O)c1cccc2c1sc1ccccc12] | 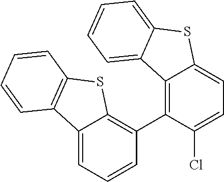 [SMILES: Clc1ccc2sc3ccccc3c2c1-c1cccc2c1sc1ccccc12] | 63.6 |
| IM-A17 | 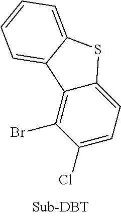 [SMILES: Clc1ccc2sc3ccccc3c2c1Br] | 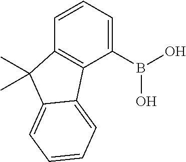 [SMILES: CC1(C)c2ccccc2-c2c(B(O)O)cccc21] | 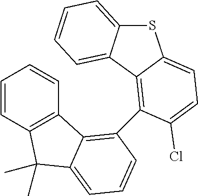 [SMILES: CC1(C)c2ccccc2-c2c(-c3c(Cl)ccc4sc5ccccc5c34)cccc21] | 55.2 |

### 4. Synthesis of Intermediate IM-A18

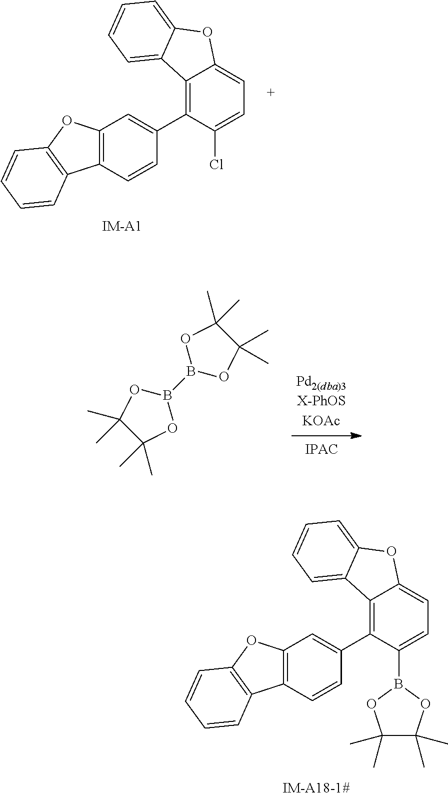

(1) Under nitrogen protection, to a reaction flask was added intermediate IM-A1 (15.0 g, 40.67 mmol), biboronic acid pinacol ester (12.4 g, 48.80 mmol), tris(dibenzylidene acetone) dipalladium (0.74 g, 0.81 mmol), 2-dicyclohexylphosphino-2′,4′,6′-triisopropylbiphenyl (0.78 g, 1.63 mmol), potassium acetate (6.0 g, 61.01 mmol) and isopropyl acetate (IPAC, 150 mL). The resulting mixture was heated to 85° C. to 90° C. under nitrogen protection and stirred for 24 hours, and then was allowed to cool to room temperature to precipitate the product out. The product was filtered out and washed until neutral. The resulting product was dissolved in toluene and passed through a silica gel chromatography column. The solvent was then evaporated under reduced pressure and purified by recrystallization from toluene to yield an intermediate IM-A18-1 #(12.45 g, yield 66.5%).

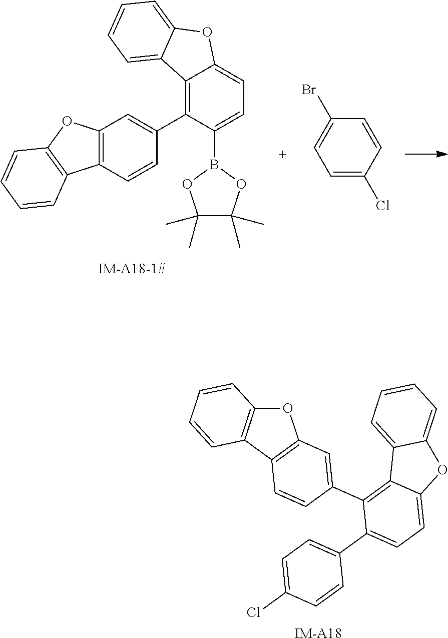

(2) Under nitrogen protection, to a three-neck flask was added the intermediate IM-A18-1 #(12.0 g, 26.07 mmol), p-chlorobromobenzene (4.99 g, 26.07 mmol), tetrabutylammonium bromide (TBAB, 1.68 g, 5.21 mmol), potassium carbonate (7.93 g, 57.35 mmol), toluene (100 mL), ethanol (40 mL) and water (20 mL). The resulting mixture was stirred and heated to 50° C. to 60° C., followed by addition of tetrakis(triphenylphosphine)palladium (0.6 g, 0.52 mmol) rapidly. After addition, the resulting mixture was heated to 70° C. to 75° C. for 12 hours of reaction. As the reaction was completed, the reaction mixture was allowed to cool to room temperature, and extracted with dichloromethane, and the resulting organic phase was washed with water until neutral, followed by being dried, filtered, and the filtrate was concentrated in vacuum to obtain a crude product. The resulting crude product was recrystallized with a mixed solvent of dichloromethane and absolute ethanol (volume ratio of 1:2). Finally, a white solid intermediate IM-A18 (8.2 g, yield 70.7%) was obtained after being dried.

### 5. Synthesis of Intermediates IM-A19 to IM-A27

Intermediates IM-Ay listed in Table 2 were synthesized following the preparation method of IM-A18, except that IM-Ax was used instead of IM-A1 to first synthesize the corresponding product IM-Ay-1 #; then, raw material 3 was used instead of p-chlorobromobenzene to react with IM-Ay-1 # to obtain the product IM-Ay. The main raw materials used, the synthesized intermediates, and their yields are shown in Table 2:

TABLE 2

| Intermediate | Raw Material | Product | | Raw | | |
|---|---|---|---|---|---|---|
| No. | IM-Ax | IM-Ay-1# | Yield/% | Material 3 | Product IM-Ay | Yield/% |
| IM-A19 | 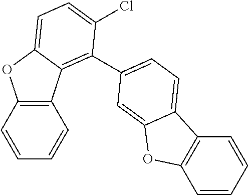 [SMILES: Clc1ccc2oc3ccccc3c2c1-c1ccc2c(c1)oc1ccccc12] | 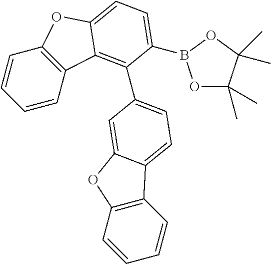 [SMILES: CC1(C)OB(c2ccc3oc4ccccc4c3c2-c2ccc3c(c2)oc2ccccc23)OC1(C)C] | 66.5 | 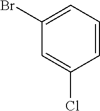 | 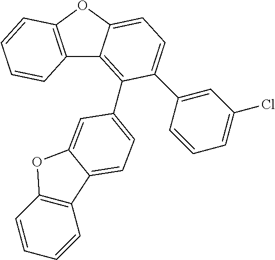 [SMILES: Clc1cccc(-c2ccc3oc4ccccc4c3c2-c2ccc3c(c2)oc2ccccc23)c1] | 68.8 |
| IM-A20 | 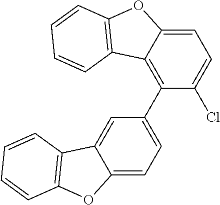 [SMILES: Clc1ccc2oc3ccccc3c2c1-c1ccc2oc3ccccc3c2c1] | 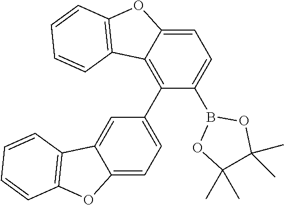 [SMILES: CC1(C)OB(c2ccc3oc4ccccc4c3c2-c2ccc3oc4ccccc4c3c2)OC1(C)C] | 67.8 | 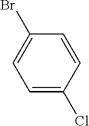 | 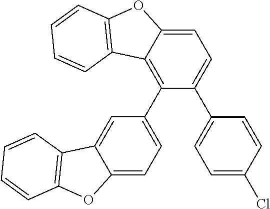 [SMILES: Clc1ccc(-c2ccc3oc4ccccc4c3c2-c2ccc3oc4ccccc4c3c2)cc1] | 72.0 |
| IM-A21 | 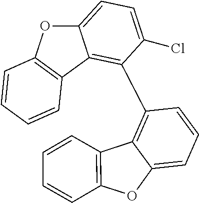 [SMILES: Clc1ccc2oc3ccccc3c2c1-c1cccc2oc3ccccc3c12] | 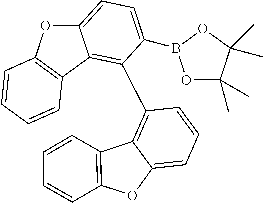 [SMILES: CC1(C)OB(c2ccc3oc4ccccc4c3c2-c2cccc3oc4ccccc4c23)OC1(C)C] | 60.9 | 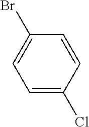 | 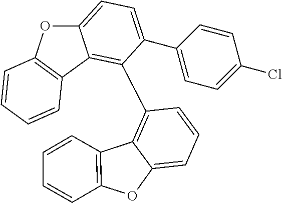 [SMILES: Clc1ccc(-c2ccc3oc4ccccc4c3c2-c2cccc3oc4ccccc4c23)cc1] | 67.7 |
| IM-A22 | 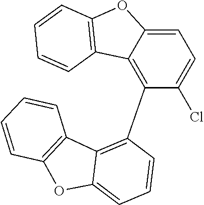 [SMILES: Clc1ccc2oc3ccccc3c2c1-c1cccc2oc3ccccc3c12] | 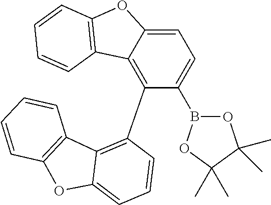 [SMILES: CC1(C)OB(c2ccc3oc4ccccc4c3c2-c2cccc3oc4ccccc4c23)OC1(C)C] | 61.0 | 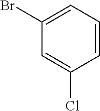 | 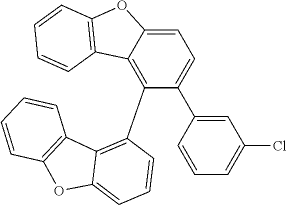 [SMILES: Clc1cccc(-c2ccc3oc4ccccc4c3c2-c2cccc3oc4ccccc4c23)c1] | 65.3 |
| IM-A23 | 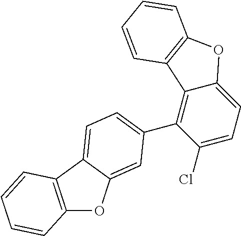 [SMILES: Clc1ccc2oc3ccccc3c2c1-c1ccc2c(c1)oc1ccccc12] | 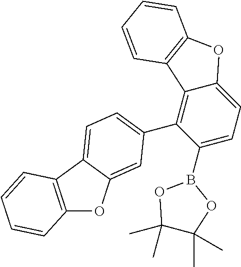 [SMILES: CC1(C)OB(c2ccc3oc4ccccc4c3c2-c2ccc3c(c2)oc2ccccc23)OC1(C)C] | 66.5 | 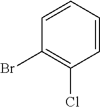 | 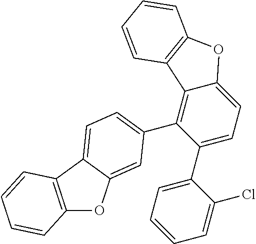 [SMILES: Clc1ccccc1-c1ccc2oc3ccccc3c2c1-c1ccc2c(c1)oc1ccccc12] | 65.8 |
| IM-A24 | 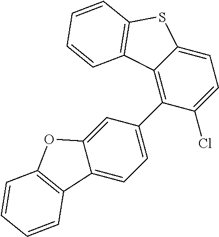 [SMILES: Clc1ccc2sc3ccccc3c2c1-c1ccc2c(c1)oc1ccccc12] | 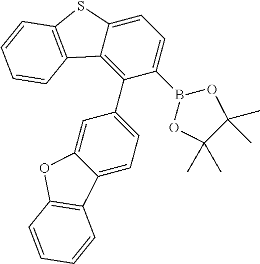 [SMILES: CC1(C)OB(c2ccc3sc4ccccc4c3c2-c2ccc3c(c2)oc2ccccc23)OC1(C)C] | 64.8 | 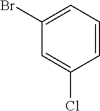 | 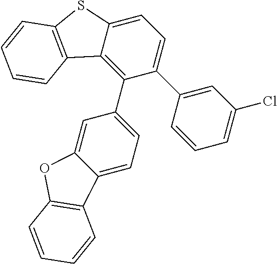 [SMILES: Clc1cccc(-c2ccc3sc4ccccc4c3c2-c2ccc3c(c2)oc2ccccc23)c1] | 70.8 |
| IM-A25 | 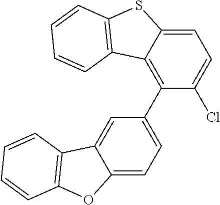 [SMILES: Clc1ccc2sc3ccccc3c2c1-c1ccc2oc3ccccc3c2c1] | 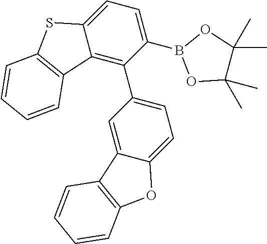 [SMILES: CC1(C)OB(c2ccc3sc4ccccc4c3c2-c2ccc3oc4ccccc4c3c2)OC1(C)C] | 67.2 | 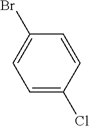 | 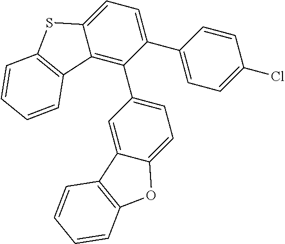 [SMILES: Clc1ccc(-c2ccc3sc4ccccc4c3c2-c2ccc3oc4ccccc4c3c2)cc1] | 72.2 |
| IM-A26 | 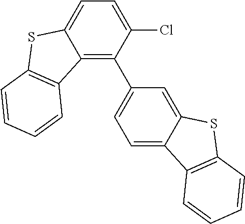 [SMILES: Clc1ccc2sc3ccccc3c2c1-c1ccc2c(c1)sc1ccccc12] | 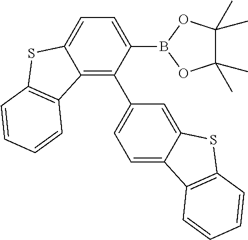 [SMILES: CC1(C)OB(c2ccc3sc4ccccc4c3c2-c2ccc3c(c2)sc2ccccc23)OC1(C)C] | 69.5 | 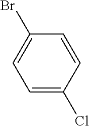 | 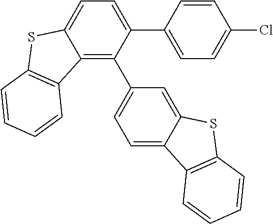 [SMILES: Clc1ccc(-c2ccc3sc4ccccc4c3c2-c2ccc3c(c2)sc2ccccc23)cc1] | 68.9 |
| IM-A27 | 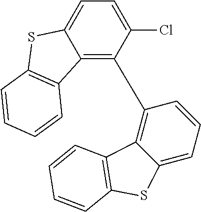 [SMILES: Clc1ccc2sc3ccccc3c2c1-c1cccc2sc3ccccc3c12] | 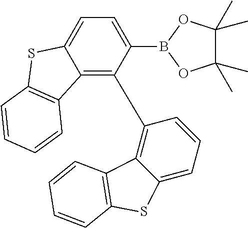 [SMILES: CC1(C)OB(c2ccc3sc4ccccc4c3c2-c2cccc3sc4ccccc4c23)OC1(C)C] | 61.1 | 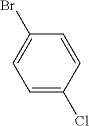 | 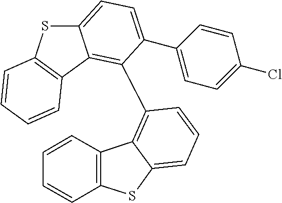 [SMILES: Clc1ccc(-c2ccc3sc4ccccc4c3c2-c2cccc3sc4ccccc4c23)cc1] | 62.5 |

### 6. Synthesis of Intermediate IM-N1

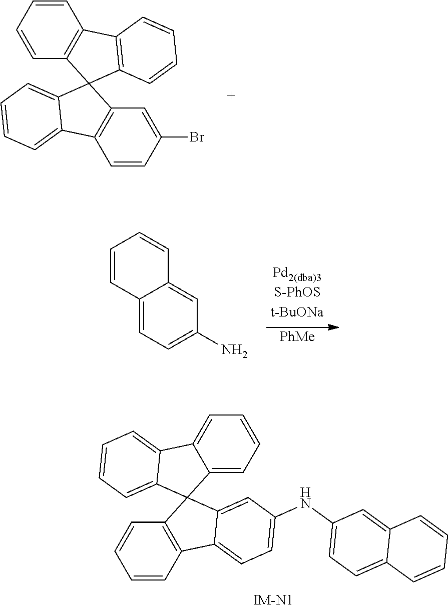 [SMILES: Brc1ccc2c(c1)C1(c3ccccc3-c3ccccc31)c1ccccc1-2 ; Nc1ccc2ccccc2c1 ; "PhMe" ; c1ccc2c(c1)-c1ccccc1C21c2ccccc2-c2ccc(Nc3ccc4ccccc4c3)cc21]

Under nitrogen protection, to a reaction flask was added 2-bromo-9,9-spirobifluorene (8.5 g, 21.50 mmol), 2-naphthylamine (3.1 g, 21.72 mmol), tris(dibenzylidene acetone) dipalladium (0.2 g, 0.22 mmol), 2-dicyclohexylphosphino-2′,6′-dimethoxybiphenyl (0.21 g, 0.43 mmol), sodium tert-butoxide (3.1 g, 32.25 mmol), and toluene (90 mL). The resulting solution was heated to 108° C. under nitrogen protection and stirred for 2 hours. After being cooled to room temperature, the reaction solution was washed with water and then dried on anhydrous magnesium sulfate. The resulting solution was filtered, and the filtrate was concentrated in vacuum to yield a crude product as a yellow solid. The crude product was then purified by recrystallization from toluene to yield IM-N1 (8.0 g, 81.3% yield).

### 7. Synthesis of Intermediates IM-N2 to IM-N12

Intermediates IM-Nx in Table 3 were synthesized following the method of IM-N1, except that raw material 4 was used instead of 2-bromo-9,9-spirobifluorene, and raw material 5 was used instead of 2-naphthylamine. The main raw materials used, the synthesized intermediates, and their yields are shown in Table 3.

TABLE 3

| Raw Material 4 | Raw Material 5 | Product IM-Nx | Yield/% |
|---|---|---|---|
| 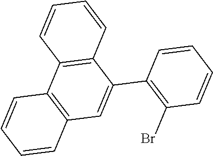 [SMILES: Brc1ccccc1-c1cc2ccccc2c2ccccc12] | 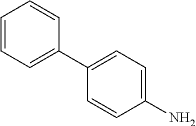 [SMILES: Nc1ccc(-c2ccccc2)cc1] | 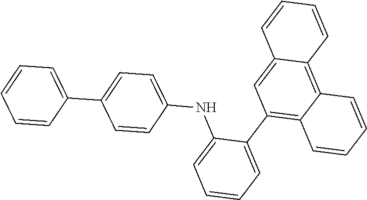 [SMILES: c1ccc(-c2ccc(Nc3ccccc3-c3cc4ccccc4c4ccccc34)cc2)cc1] | 67.2 |
| | | IM-N2 | |
| 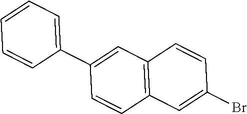 [SMILES: Brc1ccc2cc(-c3ccccc3)ccc2c1] | 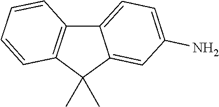 [SMILES: CC1(C)c2ccccc2-c2ccc(N)cc21] | 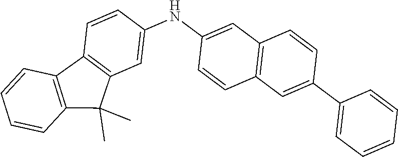 [SMILES: CC1(C)c2ccccc2-c2ccc(Nc3ccc4cc(-c5ccccc5)ccc4c3)cc21] | 72.6 |
| | | IM-N3 | |
| 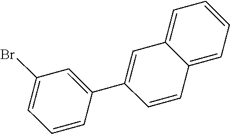 [SMILES: Brc1cccc(-c2ccc3ccccc3c2)c1] | 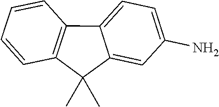 [SMILES: CC1(C)c2ccccc2-c2ccc(N)cc21] | 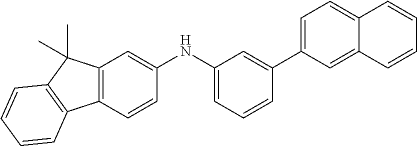 [SMILES: CC1(C)c2ccccc2-c2ccc(Nc3cccc(-c4ccc5ccccc5c4)c3)cc21] | 75.4 |
| | | IM-N4 | |
| 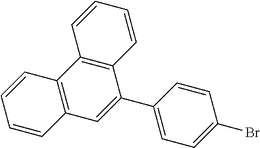 [SMILES: Brc1ccc(-c2cc3ccccc3c3ccccc23)cc1] | 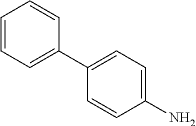 [SMILES: Nc1ccc(-c2ccccc2)cc1] | 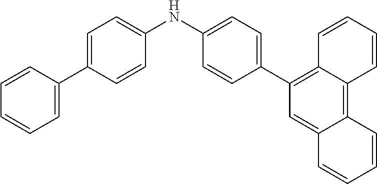 [SMILES: c1ccc(-c2ccc(Nc3ccc(-c4cc5ccccc5c5ccccc45)cc3)cc2)cc1] | 72.5 |
| | | IM-N5 | |
| 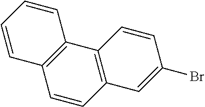 [SMILES: Brc1ccc2c(ccc3ccccc32)c1] | 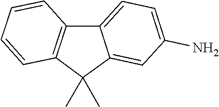 [SMILES: CC1(C)c2ccccc2-c2ccc(N)cc21] | 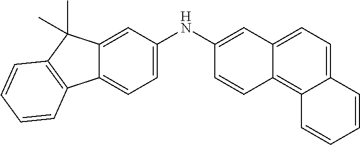 [SMILES: CC1(C)c2ccccc2-c2ccc(Nc3ccc4c(ccc5ccccc54)c3)cc21] | 70.3 |
| | | IM-N12 | |
| 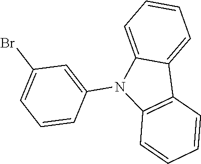 [SMILES: Brc1cccc(-n2c3ccccc3c3ccccc32)c1] | 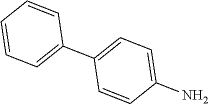 [SMILES: Nc1ccc(-c2ccccc2)cc1] | 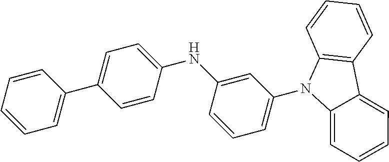 [SMILES: c1ccc(-c2ccc(Nc3cccc(-n4c5ccccc5c5ccccc54)c3)cc2)cc1] | 77.2 |
| | | IM-N6 | |
| 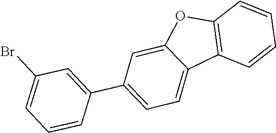 [SMILES: Brc1cccc(-c2ccc3c(c2)oc2ccccc23)c1] | 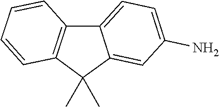 [SMILES: CC1(C)c2ccccc2-c2ccc(N)cc21] | 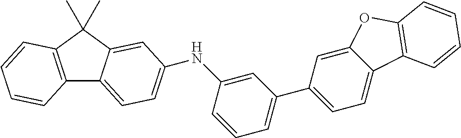 [SMILES: CC1(C)c2ccccc2-c2ccc(Nc3cccc(-c4ccc5c(c4)oc4ccccc45)c3)cc21] | 68.8 |
| | | IM-N7 | |
| 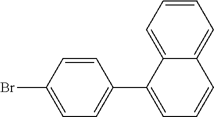 [SMILES: Brc1ccc(-c2cccc3ccccc23)cc1] | 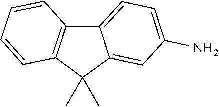 [SMILES: CC1(C)c2ccccc2-c2ccc(N)cc21] | 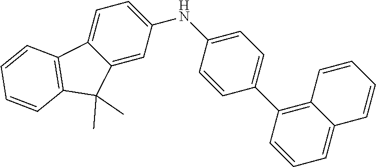 [SMILES: CC1(C)c2ccccc2-c2ccc(Nc3ccc(-c4cccc5ccccc45)cc3)cc21] | 78.5 |
| | | IM-N8 | |
| 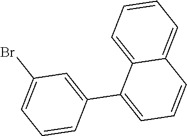 [SMILES: Brc1cccc(-c2cccc3ccccc23)c1] | 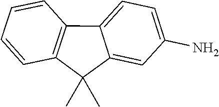 [SMILES: CC1(C)c2ccccc2-c2ccc(N)cc21] | 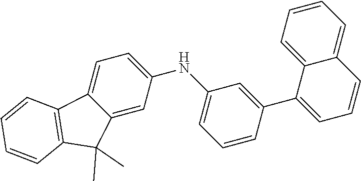 [SMILES: CC1(C)c2ccccc2-c2ccc(Nc3cccc(-c4cccc5ccccc45)c3)cc21] | 68.9 |
| | | IM-N9 | |
| 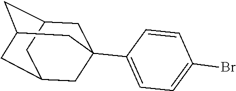 [SMILES: Brc1ccc(C23CC4CC(CC(C4)C2)C3)cc1] | 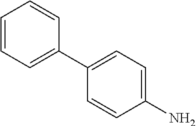 [SMILES: Nc1ccc(-c2ccccc2)cc1] | 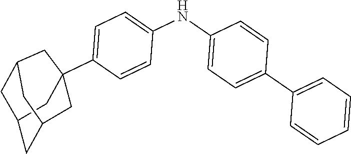 [SMILES: c1ccc(-c2ccc(Nc3ccc(C45CC6CC(CC(C6)C4)C5)cc3)cc2)cc1] | 76.8 |
| | | IM-N10 | |
| 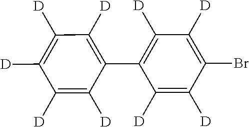 [SMILES: [2H]c1c([2H])c([2H])c(-c2c([2H])c([2H])c(Br)c([2H])c2[2H])c([2H])c1[2H]] | 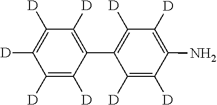 [SMILES: [2H]c1c([2H])c([2H])c(-c2c([2H])c([2H])c(N)c([2H])c2[2H])c([2H])c1[2H]] | 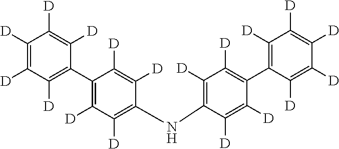 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 72.0 |
| | | IM-N11 | |

### 8. Synthesis Example 1: Synthesis of Compound 8

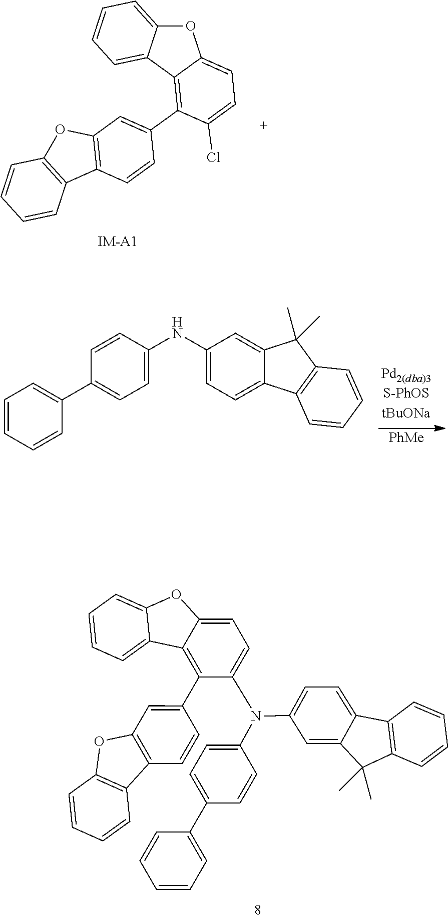

Under nitrogen protection, to a round-bottom flask was added IM-A1 (5.5 g, 14.91 mmol), N-[1,1′-biphenyl-4-yl]-9,9-dimethyl-9H-fluorenyl-2-amine (5.4 g, 14.91 mmol), tris(dibenzylidene acetone) dipalladium (0.14 g, 0.15 mmol), 2-dicyclohexylphosphino-2′,6′-dimethoxybiphenyl (0.12 g, 0.30 mmol), sodium tert-butoxide (2.15 g, 22.37 mmol), and toluene (50 mL) in turn. The resulting solution was heated to 108° C. and stirred for 6 hours. After being cooled to room temperature, the reaction solution was washed with water, and dried on magnesium sulfate. After filtration, the filtrate was concentrated in vacuum to obtain a crude product. The resulting crude product was purified by recrystallization from toluene to yield the compound 8 as a white solid (5.2 g, yield 50.5%), with a mass spectrum (m/z): m/z=694.3 [M+H]⁺.

### 9. Synthesis Example 2 to Synthesis Example 71

The compounds listed in Table 4 were synthesized following the method for synthesizing compound 8, except that raw material 6 was used instead of the intermediate IM-A1, and raw material 7 was used instead of N-[1,1′-biphenyl-4-yl]-9,9-dimethyl-9H-fluorenyl-2-amine. The main raw materials used, the synthesized compounds, their yields, and mass spectra are shown in Table 4.

TABLE 4

| Synthesis | | | | | |
|---|---|---|---|---|---|
| Example | | | | Yield/ | Mass |
| No. | Raw Material 6 | Raw Material 7 | Product | % | Spectra |
| Synthesis Example 2 | 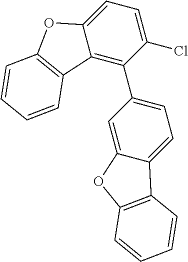 [SMILES: Clc1ccc2oc3ccccc3c2c1-c1ccc2c(c1)oc1ccccc12] | 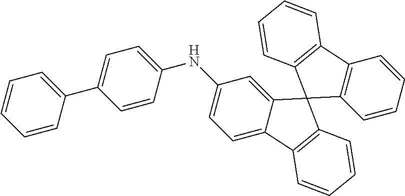 [SMILES: c1ccc(-c2ccc(Nc3ccc4c(c3)C3(c5ccccc5-c5ccccc53)c3ccccc3-4)cc2)cc1] | 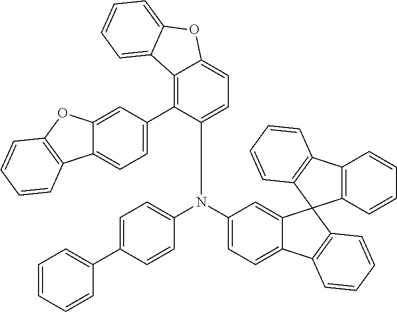 [SMILES: c1ccc(-c2ccc(N(c3ccc4c(c3)C3(c5ccccc5-c5ccccc53)c3ccccc3-4)c3ccc4oc5ccccc5c4c3-c3ccc4c(c3)oc3ccccc34)cc2)cc1] | 39.4 | 816.3 |
| | IM-A1 | CAS:2320493-46-5 | 10 | | |
| Synthesis Example 3 | 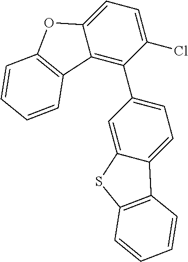 [SMILES: Clc1ccc2oc3ccccc3c2c1-c1ccc2c(c1)sc1ccccc12] | 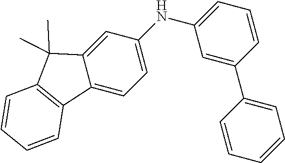 [SMILES: CC1(C)c2ccccc2-c2ccc(Nc3cccc(-c4ccccc4)c3)cc21] | 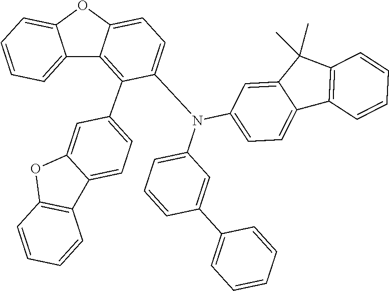 [SMILES: CC1(C)c2ccccc2-c2ccc(N(c3cccc(-c4ccccc4)c3)c3ccc4oc5ccccc5c4c3-c3ccc4c(c3)oc3ccccc34)cc21] | 38.7 | 694.3 |
| | IM-A1 | CAS:1372778-66-9 | 19 | | |
| Synthesis Example 4 | 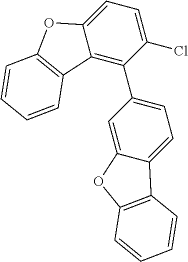 [SMILES: Clc1ccc2oc3ccccc3c2c1-c1ccc2c(c1)oc1ccccc12] | 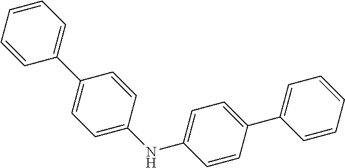 [SMILES: c1ccc(-c2ccc(Nc3ccc(-c4ccccc4)cc3)cc2)cc1] | 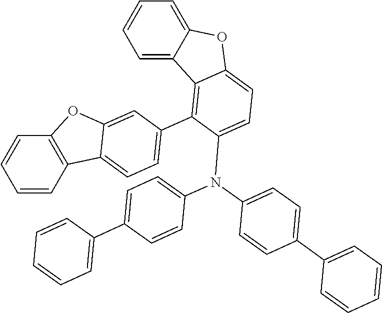 [SMILES: c1ccc(-c2ccc(N(c3ccc(-c4ccccc4)cc3)c3ccc4oc5ccccc5c4c3-c3ccc4c(c3)oc3ccccc34)cc2)cc1] | 51.6 | 654.2 |
| | IM-A1 | CAS:102113-98-4 | 21 | | |
| Synthesis Example 5 | 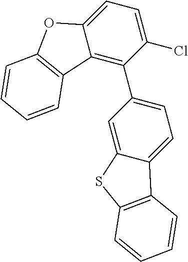 [SMILES: Clc1ccc2oc3ccccc3c2c1-c1ccc2c(c1)sc1ccccc12] | 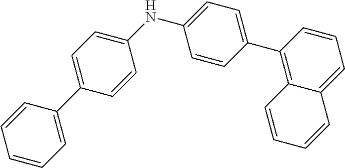 [SMILES: c1ccc(-c2ccc(Nc3ccc(-c4cccc5ccccc45)cc3)cc2)cc1] | 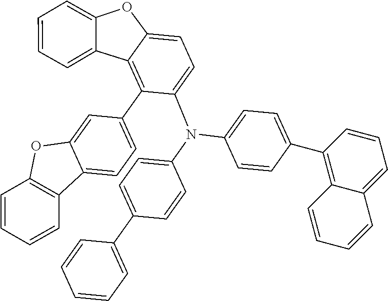 [SMILES: c1ccc(-c2ccc(N(c3ccc(-c4cccc5ccccc45)cc3)c3ccc4oc5ccccc5c4c3-c3ccc4c(c3)oc3ccccc34)cc2)cc1] | 46.1 | 704.2 |
| | IM-A1 | CAS:897921-59-4 | 25 | | |
| Synthesis Example 6 | 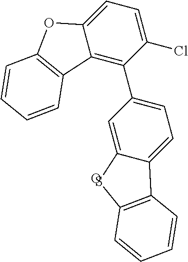 | 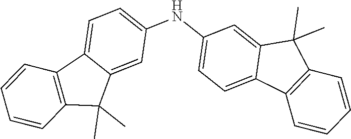 [SMILES: CC1(C)c2ccccc2-c2ccc(Nc3ccc4c(c3)C(C)(C)c3ccccc3-4)cc21] | 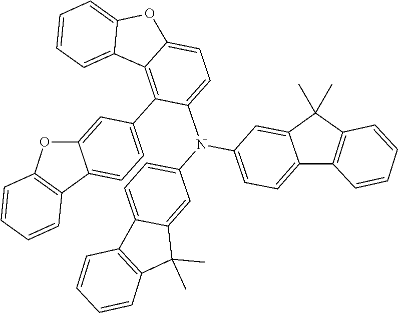 [SMILES: CC1(C)c2ccccc2-c2ccc(N(c3ccc4c(c3)C(C)(C)c3ccccc3-4)c3ccc4oc5ccccc5c4c3-c3ccc4c(c3)oc3ccccc34)cc21] | 41.5 | 734.3 |
| | IM-A1 | CAS:500717-23-7 | 31 | | |
| Synthesis Example 7 | 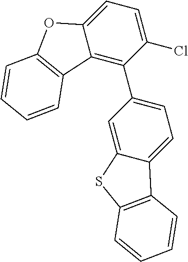 [SMILES: Clc1ccc2oc3ccccc3c2c1-c1ccc2c(c1)sc1ccccc12] | 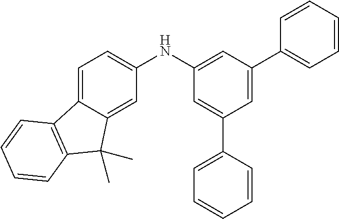 [SMILES: CC1(C)c2ccccc2-c2ccc(Nc3cc(-c4ccccc4)cc(-c4ccccc4)c3)cc21] | 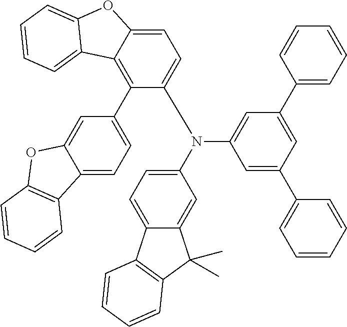 [SMILES: CC1(C)c2ccccc2-c2ccc(N(c3cc(-c4ccccc4)cc(-c4ccccc4)c3)c3ccc4oc5ccccc5c4c3-c3ccc4c(c3)oc3ccccc34)cc21] | 43.0 | 770.3 |
| | IM-A1 | CAS:1372778-68-1 | 40 | | |
| Synthesis Example 8 | 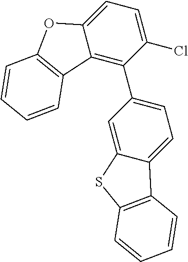 [SMILES: Clc1ccc2oc3ccccc3c2c1-c1ccc2c(c1)sc1ccccc12] | 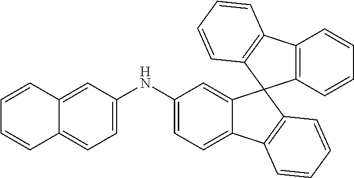 [SMILES: c1ccc2c(c1)-c1ccccc1C21c2ccccc2-c2ccc(Nc3ccc4ccccc4c3)cc21] | 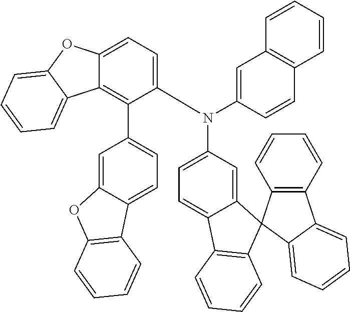 [SMILES: c1ccc2c(c1)-c1ccccc1C21c2ccccc2-c2ccc(N(c3ccc4ccccc4c3)c3ccc4oc5ccccc5c4c3-c3ccc4c(c3)oc3ccccc34)cc21] | 39.9 | 790.3 |
| | IM-A1 | IM-N1 | 44 | | |
| Synthesis Example 9 | 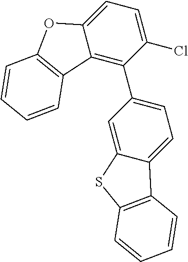 [SMILES: Clc1ccc2oc3ccccc3c2c1-c1ccc2c(c1)sc1ccccc12] | 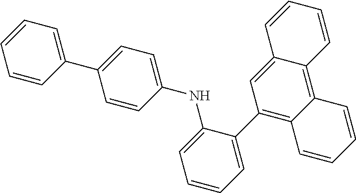 [SMILES: c1ccc(-c2ccc(Nc3ccccc3-c3cc4ccccc4c4ccccc34)cc2)cc1] | 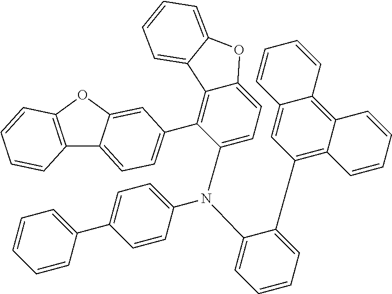 [SMILES: c1ccc(-c2ccc(N(c3ccccc3-c3cc4ccccc4c4ccccc34)c3ccc4oc5ccccc5c4c3-c3ccc4c(c3)oc3ccccc34)cc2)cc1] | 36.2 | 754.3 |
| | IM-A1 | IM-N2 | 48 | | |
| Synthesis Example 10 | 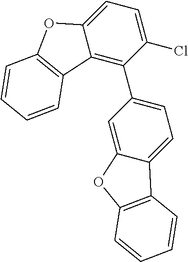 [SMILES: Clc1ccc2oc3ccccc3c2c1-c1ccc2c(c1)oc1ccccc12] | 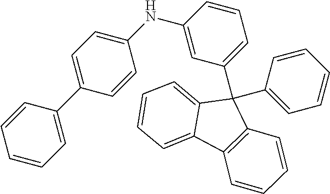 [SMILES: c1ccc(-c2ccc(Nc3cccc(C4(c5ccccc5)c5ccccc5-c5ccccc54)c3)cc2)cc1] | 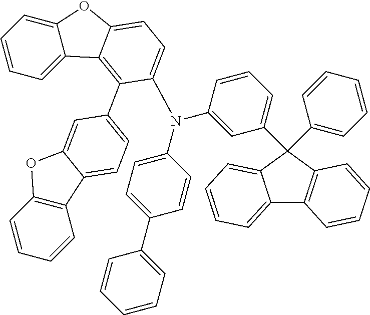 [SMILES: c1ccc(-c2ccc(N(c3cccc(C4(c5ccccc5)c5ccccc5-c5ccccc54)c3)c3ccc4oc5ccccc5c4c3-c3ccc4c(c3)oc3ccccc34)cc2)cc1] | 42.0 | 818.3 |
| | IM-A1 | CAS:2305719-96-2 | 56 | | |
| Synthesis Example 11 | 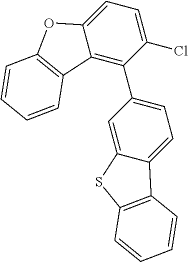 [SMILES: Clc1ccc2oc3ccccc3c2c1-c1ccc2c(c1)sc1ccccc12] | 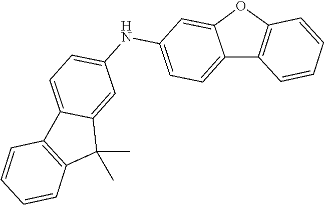 [SMILES: CC1(C)c2ccccc2-c2ccc(Nc3ccc4c(c3)oc3ccccc34)cc21] | 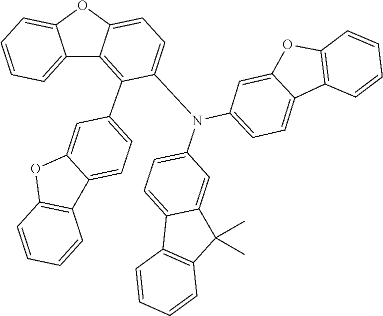 [SMILES: CC1(C)c2ccccc2-c2ccc(N(c3ccc4c(c3)oc3ccccc34)c3ccc4oc5ccccc5c4c3-c3ccc4c(c3)oc3ccccc34)cc21] | 38.6 | 708.2 |
| | IM-A1 | CAS:1427556-50-0 | 64 | | |
| Synthesis Example 12 | 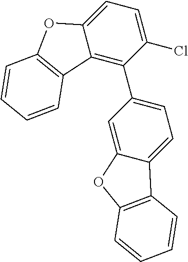 [SMILES: Clc1ccc2oc3ccccc3c2c1-c1ccc2c(c1)oc1ccccc12] | 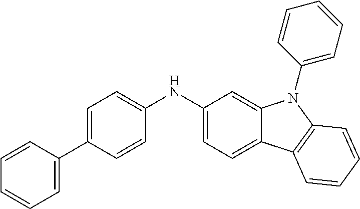 [SMILES: c1ccc(-c2ccc(Nc3ccc4c5ccccc5n(-c5ccccc5)c4c3)cc2)cc1] | 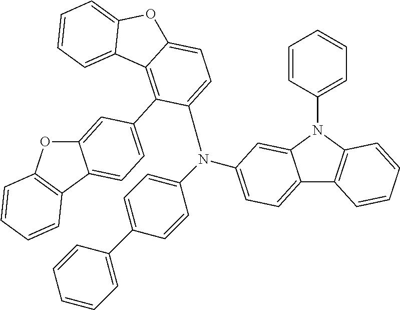 [SMILES: c1ccc(-c2ccc(N(c3ccc4c5ccccc5n(-c5ccccc5)c4c3)c3ccc4oc5ccccc5c4c3-c3ccc4c(c3)oc3ccccc34)cc2)cc1] | 47.3 | 743.3 |
| | IM-A1 | CAS:1427316-58-2 | 67 | | |
| Synthesis Example 13 | 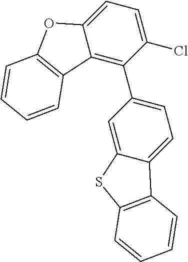 [SMILES: Clc1ccc2oc3ccccc3c2c1-c1ccc2c(c1)sc1ccccc12] | 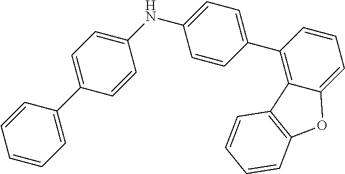 [SMILES: c1ccc(-c2ccc(Nc3ccc(-c4cccc5oc6ccccc6c45)cc3)cc2)cc1] | 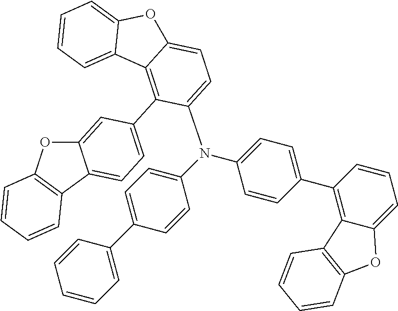 [SMILES: c1ccc(-c2ccc(N(c3ccc(-c4cccc5oc6ccccc6c45)cc3)c3ccc4oc5ccccc5c4c3-c3ccc4c(c3)oc3ccccc34)cc2)cc1] | 41.8 | 744.2 |
| | IM-A1 | CAS:1922919-50-3 | 71 | | |
| Synthesis Example 14 | 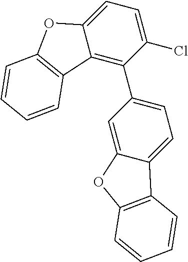 [SMILES: Clc1ccc2oc3ccccc3c2c1-c1ccc2c(c1)oc1ccccc12] | 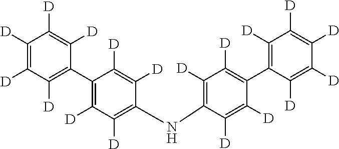 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 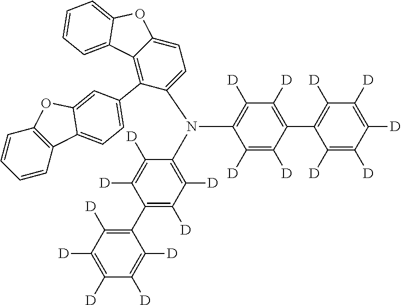 [SMILES: [2H]c1c([2H])c([2H])c(-c2c([2H])c([2H])c(N(c3ccc4oc5ccccc5c4c3-c3ccc4c(c3)oc3ccccc34)c3c([2H])c([2H])c(-c4c([2H])c([2H])c([2H])c([2H])c4[2H])c([2H])c3[2H])c([2H])c2[2H])c([2H])c1[2H]] | 44.8 | 672.4 |
| | IM-A1 | IM-N11 | 218 | | |
| Synthesis Example 15 | 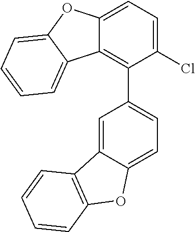 [SMILES: Clc1ccc2oc3ccccc3c2c1-c1ccc2oc3ccccc3c2c1] | 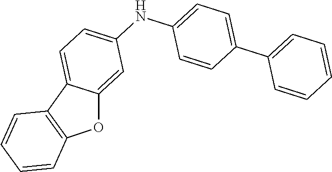 [SMILES: c1ccc(-c2ccc(Nc3ccc4c(c3)oc3ccccc34)cc2)cc1] | 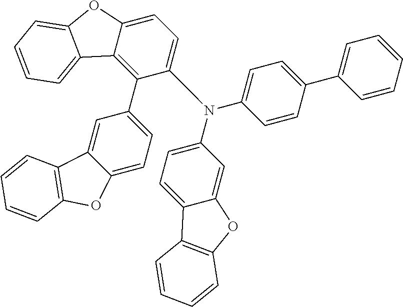 [SMILES: c1ccc(-c2ccc(N(c3ccc4c(c3)oc3ccccc34)c3ccc4oc5ccccc5c4c3-c3ccc4oc5ccccc5c4c3)cc2)cc1] | 44.0 | 668.2 |
| | IM-A3 | CAS:1290039-85-8 | 93 | | |
| Synthesis Example 16 | 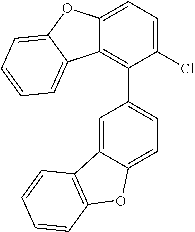 [SMILES: Clc1ccc2oc3ccccc3c2c1-c1ccc2oc3ccccc3c2c1] | 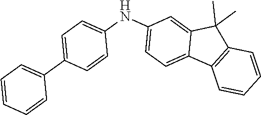 [SMILES: CC1(C)c2ccccc2-c2ccc(Nc3ccc(-c4ccccc4)cc3)cc21] | 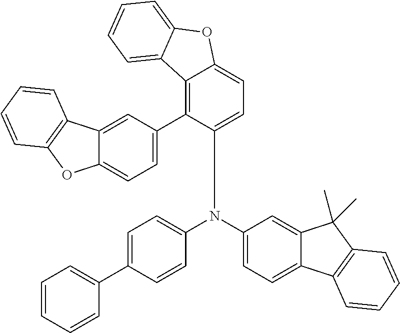 [SMILES: CC1(C)c2ccccc2-c2ccc(N(c3ccc(-c4ccccc4)cc3)c3ccc4oc5ccccc5c4c3-c3ccc4oc5ccccc5c4c3)cc21] | 47.7 | 694.3 |
| | IM-A3 | CAS:897671-69-1 | 94 | | |
| Synthesis Example 17 | 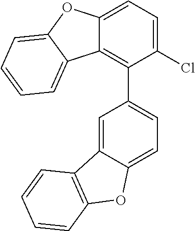 [SMILES: Clc1ccc2oc3ccccc3c2c1-c1ccc2oc3ccccc3c2c1] | 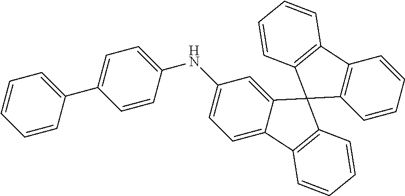 [SMILES: c1ccc(-c2ccc(Nc3ccc4c(c3)C3(c5ccccc5-c5ccccc53)c3ccccc3-4)cc2)cc1] | 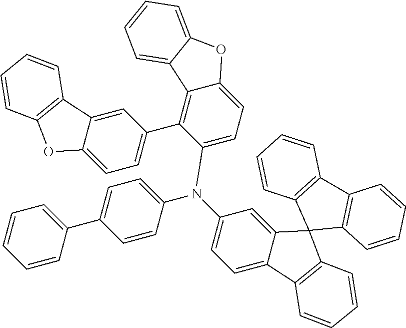 [SMILES: c1ccc(-c2ccc(N(c3ccc4c(c3)C3(c5ccccc5-c5ccccc53)c3ccccc3-4)c3ccc4oc5ccccc5c4c3-c3ccc4oc5ccccc5c4c3)cc2)cc1] | 37.5 | 816.3 |
| | IM-A3 | CAS:1258514-95-2 | 95 | | |
| Synthesis Example 18 | 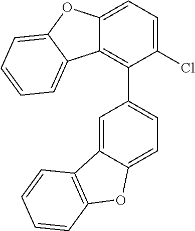 [SMILES: Clc1ccc2oc3ccccc3c2c1-c1ccc2oc3ccccc3c2c1] | 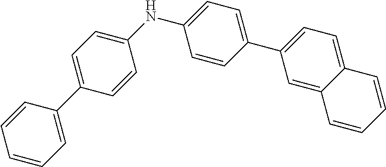 [SMILES: c1ccc(-c2ccc(Nc3ccc(-c4ccc5ccccc5c4)cc3)cc2)cc1] | 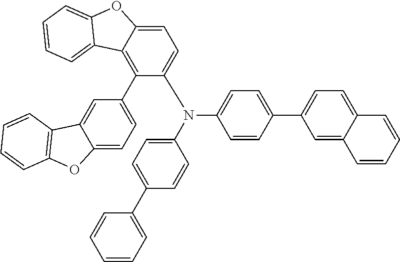 [SMILES: c1ccc(-c2ccc(N(c3ccc(-c4ccc5ccccc5c4)cc3)c3ccc4oc5ccccc5c4c3-c3ccc4oc5ccccc5c4c3)cc2)cc1] | 46.0 | 704.2 |
| | IM-A3 | CAS:897921-60-7 | 111 | | |
| Synthesis Example 19 | 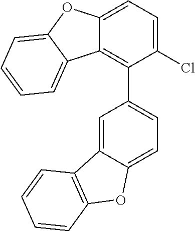 [SMILES: Clc1ccc2oc3ccccc3c2c1-c1ccc2oc3ccccc3c2c1] | 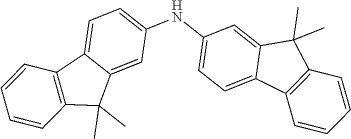 [SMILES: CC1(C)c2ccccc2-c2ccc(Nc3ccc4c(c3)C(C)(C)c3ccccc3-4)cc21] | 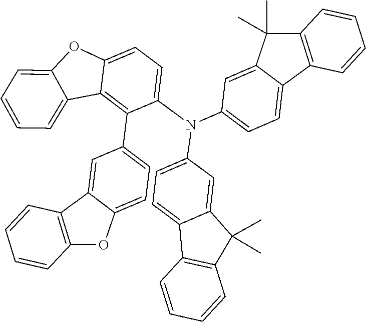 [SMILES: CC1(C)c2ccccc2-c2ccc(N(c3ccc4c(c3)C(C)(C)c3ccccc3-4)c3ccc4oc5ccccc5c4c3-c3ccc4oc5ccccc5c4c3)cc21] | 43.2 | 734.3 |
| | IM-A3 | CAS:500717-23-7 | 116 | | |
| Synthesis Example 20 | 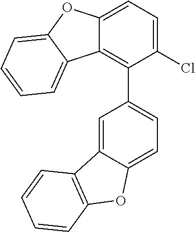 [SMILES: Clc1ccc2oc3ccccc3c2c1-c1ccc2oc3ccccc3c2c1] | 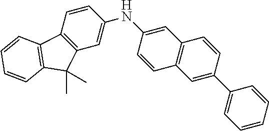 [SMILES: CC1(C)c2ccccc2-c2ccc(Nc3ccc4cc(-c5ccccc5)ccc4c3)cc21] | 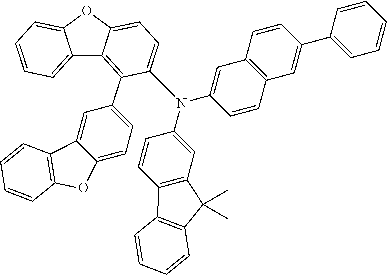 [SMILES: CC1(C)c2ccccc2-c2ccc(N(c3ccc4cc(-c5ccccc5)ccc4c3)c3ccc4oc5ccccc5c4c3-c3ccc4oc5ccccc5c4c3)cc21] | 38.9 | 744.3 |
| | IM-A3 | IM-N3 | 119 | | |
| Synthesis Example 21 | 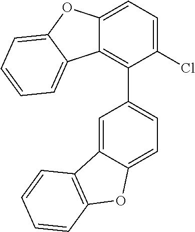 [SMILES: Clc1ccc2oc3ccccc3c2c1-c1ccc2oc3ccccc3c2c1] | 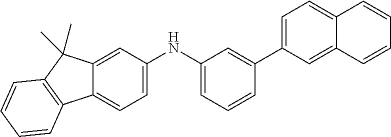 [SMILES: CC1(C)c2ccccc2-c2ccc(Nc3cccc(-c4ccc5ccccc5c4)c3)cc21] | 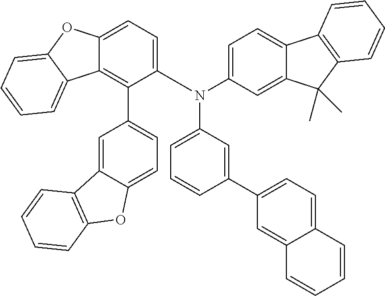 [SMILES: CC1(C)c2ccccc2-c2ccc(N(c3cccc(-c4ccc5ccccc5c4)c3)c3ccc4oc5ccccc5c4c3-c3ccc4oc5ccccc5c4c3)cc21] | 40.8 | 744.3 |
| | IM-A3 | IM-N4 | 123 | | |
| Synthesis Example 22 | 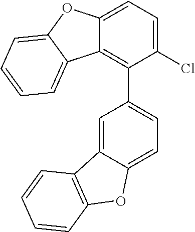 [SMILES: Clc1ccc2oc3ccccc3c2c1-c1ccc2oc3ccccc3c2c1] | 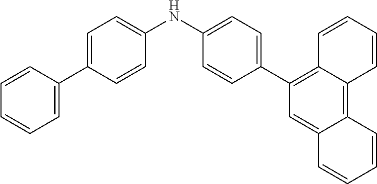 [SMILES: c1ccc(-c2ccc(Nc3ccc(-c4cc5ccccc5c5ccccc45)cc3)cc2)cc1] | 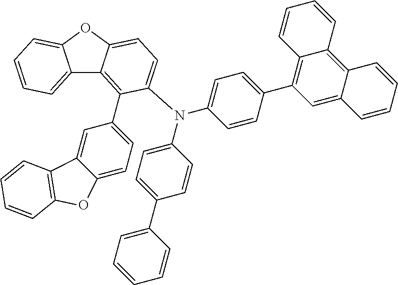 [SMILES: c1ccc(-c2ccc(N(c3ccc(-c4cc5ccccc5c5ccccc45)cc3)c3ccc4oc5ccccc5c4c3-c3ccc4oc5ccccc5c4c3)cc2)cc1] | 46.8 | 754.3 |
| | IM-A3 | IM-N5 | 131 | | |
| Synthesis Example 23 | 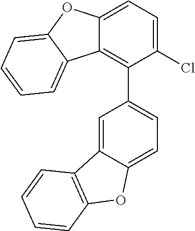 [SMILES: Clc1ccc2oc3ccccc3c2c1-c1ccc2oc3ccccc3c2c1] | 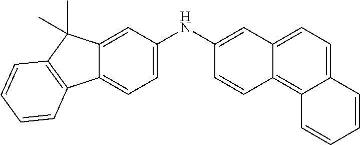 [SMILES: CC1(C)c2ccccc2-c2ccc(Nc3ccc4c(ccc5ccccc54)c3)cc21] | 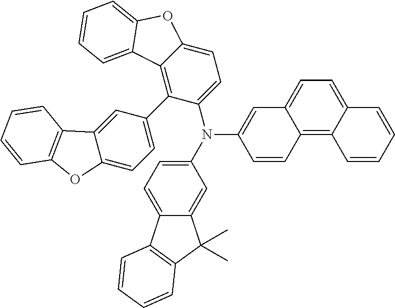 [SMILES: CC1(C)c2ccccc2-c2ccc(N(c3ccc4c(ccc5ccccc54)c3)c3ccc4oc5ccccc5c4c3-c3ccc4oc5ccccc5c4c3)cc21] | 38.5 | 718.3 |
| | IM-A3 | IM-N12 | 140 | | |
| Synthesis Example 24 | 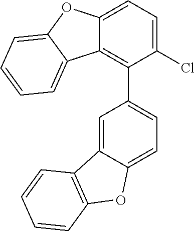 [SMILES: Clc1ccc2oc3ccccc3c2c1-c1ccc2oc3ccccc3c2c1] | 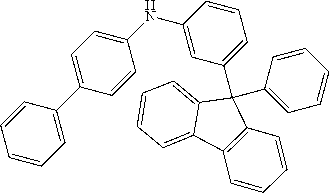 [SMILES: c1ccc(-c2ccc(Nc3cccc(C4(c5ccccc5)c5ccccc5-c5ccccc54)c3)cc2)cc1] | 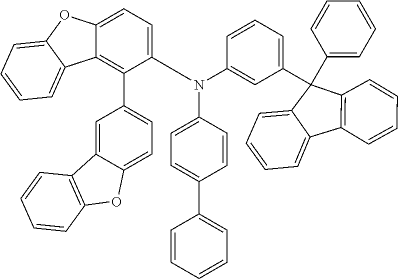 [SMILES: c1ccc(-c2ccc(N(c3cccc(C4(c5ccccc5)c5ccccc5-c5ccccc54)c3)c3ccc4oc5ccccc5c4c3-c3ccc4oc5ccccc5c4c3)cc2)cc1] | 40.1 | 818.3 |
| | IM-A3 | CAS: 2305719-96-2 | 141 | | |
| Synthesis Example 25 | 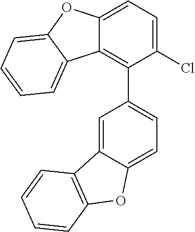 [SMILES: Clc1ccc2oc3ccccc3c2c1-c1ccc2oc3ccccc3c2c1] | 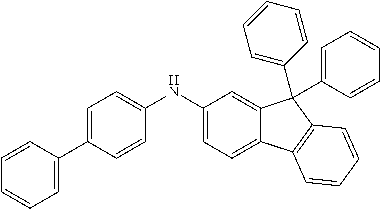 [SMILES: c1ccc(-c2ccc(Nc3ccc4c(c3)C(c3ccccc3)(c3ccccc3)c3ccccc3-4)cc2)cc1] | 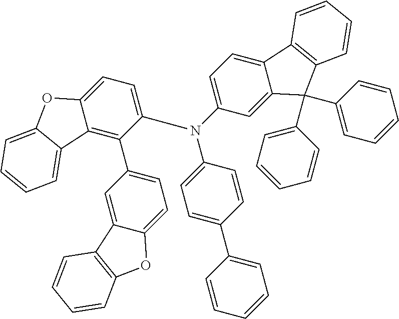 [SMILES: c1ccc(-c2ccc(N(c3ccc4c(c3)C(c3ccccc3)(c3ccccc3)c3ccccc3-4)c3ccc4oc5ccccc5c4c3-c3ccc4oc5ccccc5c4c3)cc2)cc1] | 48.7 | 818.3 |
| | IM-A3 | CAS:1268520-04-2 | 143 | | |
| Synthesis Example 26 | 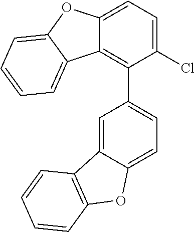 [SMILES: Clc1ccc2oc3ccccc3c2c1-c1ccc2oc3ccccc3c2c1] | 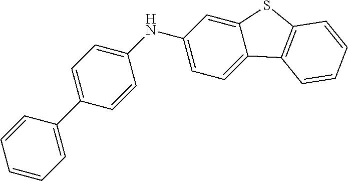 [SMILES: c1ccc(-c2ccc(Nc3ccc4c(c3)sc3ccccc34)cc2)cc1] | 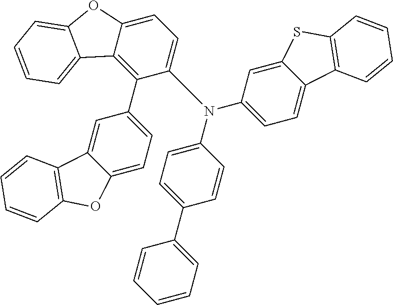 [SMILES: c1ccc(-c2ccc(N(c3ccc4c(c3)sc3ccccc34)c3ccc4oc5ccccc5c4c3-c3ccc4oc5ccccc5c4c3)cc2)cc1] | 45.8 | 684.2 |
| | IM-A3 | CAS:1290039-87-0 | 146 | | |
| Synthesis Example 27 | 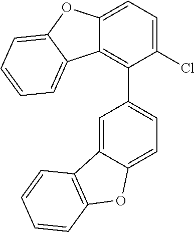 [SMILES: Clc1ccc2oc3ccccc3c2c1-c1ccc2oc3ccccc3c2c1] | 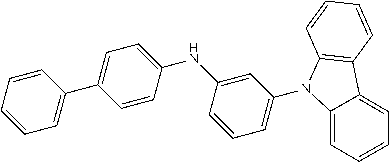 [SMILES: c1ccc(-c2ccc(Nc3cccc(-n4c5ccccc5c5ccccc54)c3)cc2)cc1] | 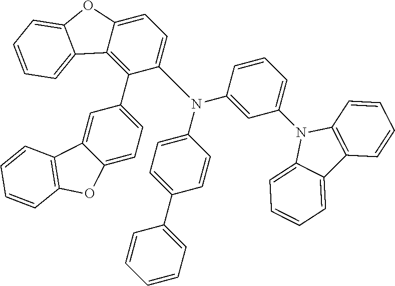 [SMILES: c1ccc(-c2ccc(N(c3cccc(-n4c5ccccc5c5ccccc54)c3)c3ccc4oc5ccccc5c4c3-c3ccc4oc5ccccc5c4c3)cc2)cc1] | 41.4 | 743.3 |
| | IM-A3 | IM-N6 | 155 | | |
| Synthesis Example 28 | 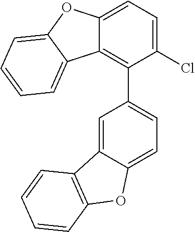 [SMILES: Clc1ccc2oc3ccccc3c2c1-c1ccc2oc3ccccc3c2c1] | 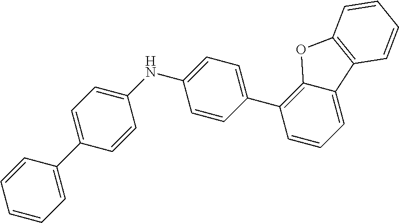 [SMILES: c1ccc(-c2ccc(Nc3ccc(-c4cccc5c4oc4ccccc45)cc3)cc2)cc1] | 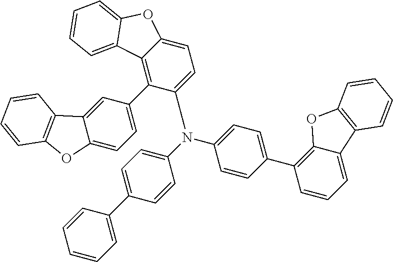 [SMILES: c1ccc(-c2ccc(N(c3ccc(-c4cccc5c4oc4ccccc45)cc3)c3ccc4oc5ccccc5c4c3-c3ccc4oc5ccccc5c4c3)cc2)cc1] | 46.4 | 744.2 |
| | IM-A3 | CAS:955959-89-4 | 157 | | |
| Synthesis Example 29 | 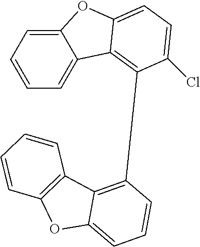 [SMILES: Clc1ccc2oc3ccccc3c2c1-c1cccc2oc3ccccc3c12] | 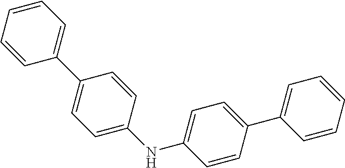 [SMILES: c1ccc(-c2ccc(Nc3ccc(-c4ccccc4)cc3)cc2)cc1] | 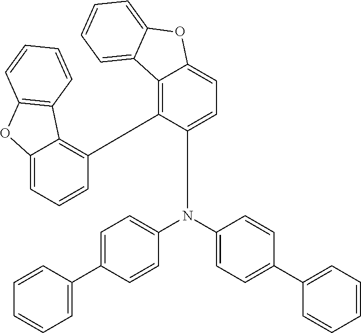 [SMILES: c1ccc(-c2ccc(N(c3ccc(-c4ccccc4)cc3)c3ccc4oc5ccccc5c4c3-c3cccc4oc5ccccc5c34)cc2)cc1] | 40.5 | 654.2 |
| | IM-A4 | CAS:102113-98-4 | 181 | | |
| Synthesis Example 30 | 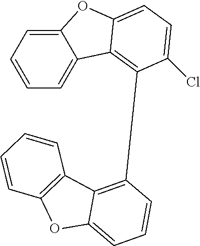 [SMILES: Clc1ccc2oc3ccccc3c2c1-c1cccc2oc3ccccc3c12] | 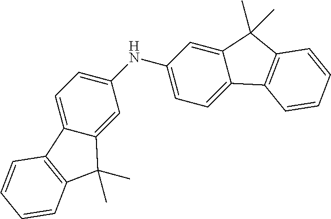 [SMILES: CC1(C)c2ccccc2-c2ccc(Nc3ccc4c(c3)C(C)(C)c3ccccc3-4)cc21] | 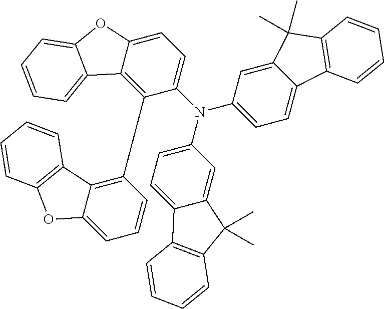 [SMILES: CC1(C)c2ccccc2-c2ccc(N(c3ccc4c(c3)C(C)(C)c3ccccc3-4)c3ccc4oc5ccccc5c4c3-c3cccc4oc5ccccc5c34)cc21] | 36.3 | 734.3 |
| | IM-A4 | CAS:500717-23-7 | 186 | | |
| Synthesis Example 31 | 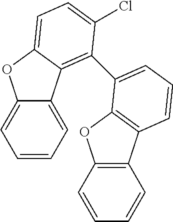 [SMILES: Clc1ccc2oc3ccccc3c2c1-c1cccc2c1oc1ccccc12] | 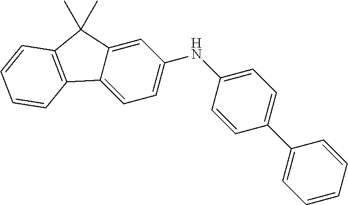 [SMILES: CC1(C)c2ccccc2-c2ccc(Nc3ccc(-c4ccccc4)cc3)cc21] | 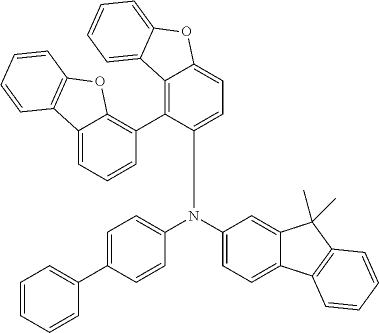 [SMILES: CC1(C)c2ccccc2-c2ccc(N(c3ccc(-c4ccccc4)cc3)c3ccc4oc5ccccc5c4c3-c3cccc4c3oc3ccccc34)cc21] | 40.4 | 694.3 |
| | IM-A5 | CAS:897671-69-1 | 219 | | |
| Synthesis Example 32 | 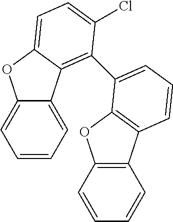 [SMILES: Clc1ccc2oc3ccccc3c2c1-c1cccc2c1oc1ccccc12] | 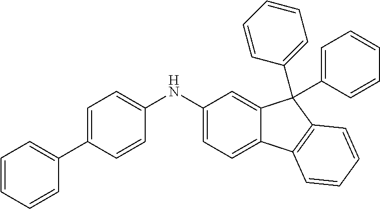 [SMILES: c1ccc(-c2ccc(Nc3ccc4c(c3)C(c3ccccc3)(c3ccccc3)c3ccccc3-4)cc2)cc1] | 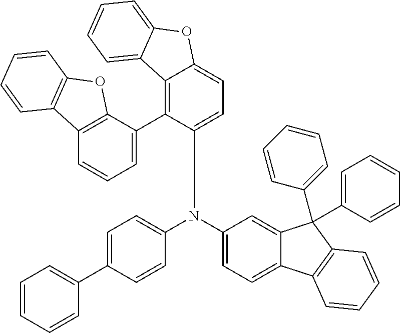 [SMILES: c1ccc(-c2ccc(N(c3ccc4c(c3)C(c3ccccc3)(c3ccccc3)c3ccccc3-4)c3ccc4oc5ccccc5c4c3-c3cccc4c3oc3ccccc34)cc2)cc1] | 36.5 | 818.3 |
| | IM-A5 | CAS:1268520-04-2 | 225 | | |
| Synthesis Example 33 | 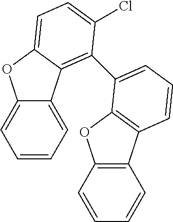 [SMILES: Clc1ccc2oc3ccccc3c2c1-c1cccc2c1oc1ccccc12] | 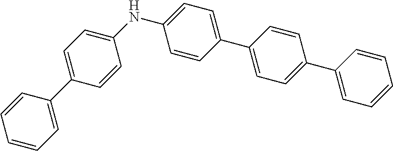 [SMILES: c1ccc(-c2ccc(Nc3ccc(-c4ccc(-c5ccccc5)cc4)cc3)cc2)cc1] | 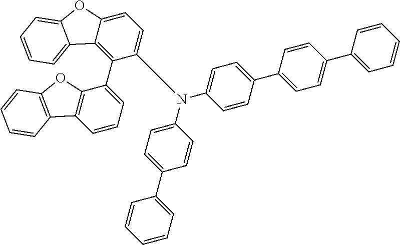 [SMILES: c1ccc(-c2ccc(-c3ccc(N(c4ccc(-c5ccccc5)cc4)c4ccc5oc6ccccc6c5c4-c4cccc5c4oc4ccccc45)cc3)cc2)cc1] | 40.4 | 730.3 |
| | IM-A5 | CAS:897921-63-0 | 251 | | |
| Synthesis Example 34 | 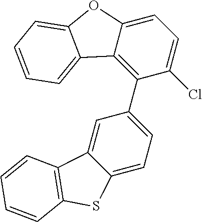 [SMILES: Clc1ccc2oc3ccccc3c2c1-c1ccc2sc3ccccc3c2c1] | 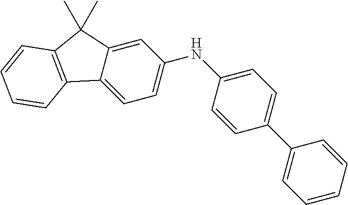 [SMILES: CC1(C)c2ccccc2-c2ccc(Nc3ccc(-c4ccccc4)cc3)cc21] | 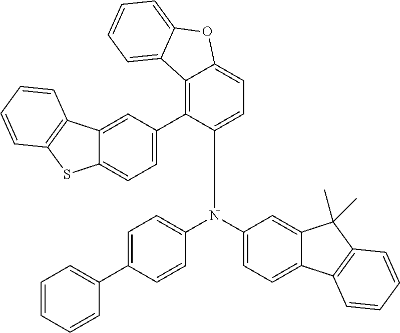 [SMILES: CC1(C)c2ccccc2-c2ccc(N(c3ccc(-c4ccccc4)cc3)c3ccc4oc5ccccc5c4c3-c3ccc4sc5ccccc5c4c3)cc21] | 49.5 | 710.2 |
| | IM-A6 | CAS:897671-69-1 | 259 | | |
| Synthesis Example 35 | 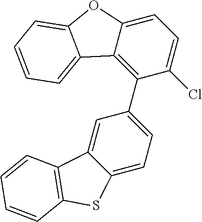 [SMILES: Clc1ccc2oc3ccccc3c2c1-c1ccc2sc3ccccc3c2c1] | 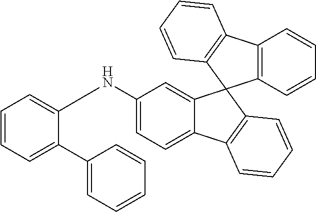 [SMILES: c1ccc(-c2ccccc2Nc2ccc3c(c2)C2(c4ccccc4-c4ccccc42)c2ccccc2-3)cc1] | 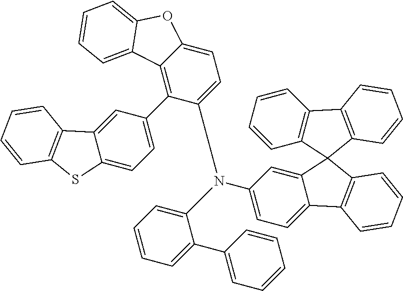 [SMILES: c1ccc(-c2ccccc2N(c2ccc3c(c2)C2(c4ccccc4-c4ccccc42)c2ccccc2-3)c2ccc3oc4ccccc4c3c2-c2ccc3sc4ccccc4c3c2)cc1] | 41.4 | 832.3 |
| | IM-A6 | CAS:1364602-55-0 | 265 | | |
| Synthesis Example 36 | 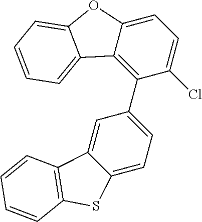 [SMILES: Clc1ccc2oc3ccccc3c2c1-c1ccc2sc3ccccc3c2c1] | 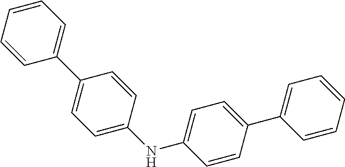 [SMILES: c1ccc(-c2ccc(Nc3ccc(-c4ccccc4)cc3)cc2)cc1] | 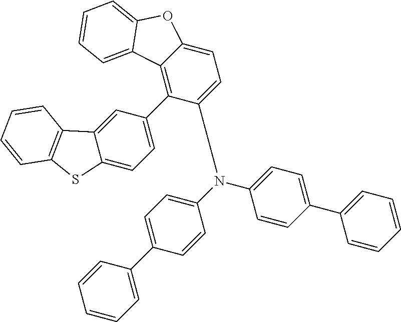 [SMILES: c1ccc(-c2ccc(N(c3ccc(-c4ccccc4)cc3)c3ccc4oc5ccccc5c4c3-c3ccc4sc5ccccc5c4c3)cc2)cc1] | 50.4 | 670.2 |
| | IM-A6 | CAS:102113-98-4 | 266 | | |
| Synthesis Example 37 | 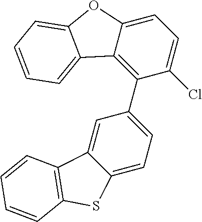 [SMILES: Clc1ccc2oc3ccccc3c2c1-c1ccc2sc3ccccc3c2c1] | 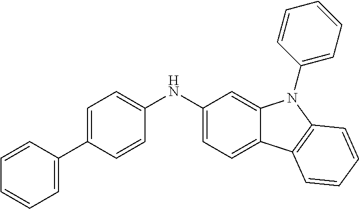 [SMILES: c1ccc(-c2ccc(Nc3ccc4c5ccccc5n(-c5ccccc5)c4c3)cc2)cc1] | 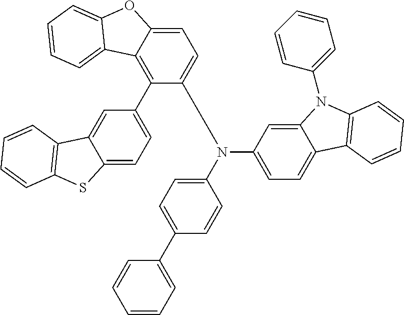 [SMILES: c1ccc(-c2ccc(N(c3ccc4c5ccccc5n(-c5ccccc5)c4c3)c3ccc4oc5ccccc5c4c3-c3ccc4sc5ccccc5c4c3)cc2)cc1] | 48.3 | 759.2 |
| | IM-A6 | CAS:1427316-58-2 | 312 | | |
| Synthesis Example 38 | 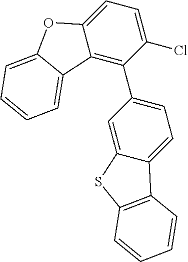 [SMILES: Clc1ccc2oc3ccccc3c2c1-c1ccc2c(c1)sc1ccccc12] | 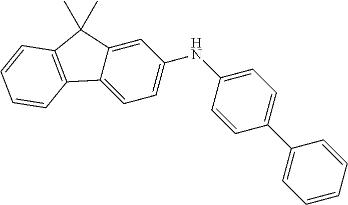 [SMILES: CC1(C)c2ccccc2-c2ccc(Nc3ccc(-c4ccccc4)cc3)cc21] | 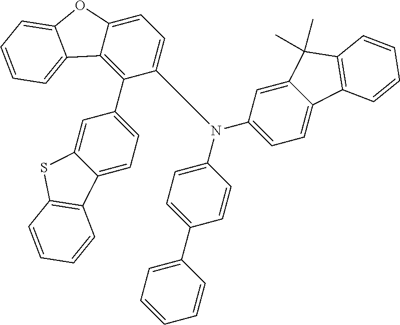 [SMILES: CC1(C)c2ccccc2-c2ccc(N(c3ccc(-c4ccccc4)cc3)c3ccc4oc5ccccc5c4c3-c3ccc4c(c3)sc3ccccc34)cc21] | 43.6 | 710.2 |
| | IM-A7 | CAS:897671-69-1 | 339 | | |
| Synthesis Example 39 | 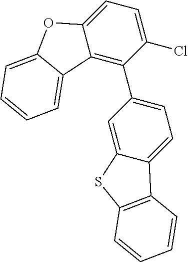 [SMILES: Clc1ccc2oc3ccccc3c2c1-c1ccc2c(c1)sc1ccccc12] | 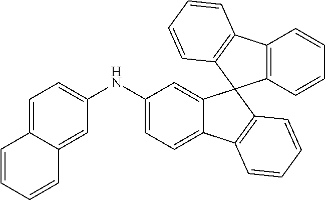 [SMILES: c1ccc2c(c1)-c1ccccc1C21c2ccccc2-c2ccc(Nc3ccc4ccccc4c3)cc21] | 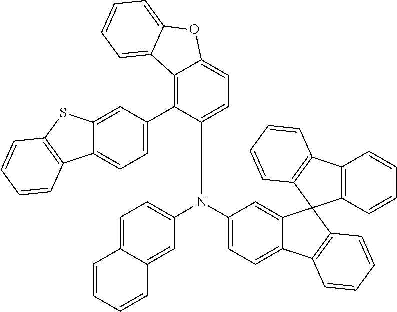 [SMILES: c1ccc2c(c1)-c1ccccc1C21c2ccccc2-c2ccc(N(c3ccc4ccccc4c3)c3ccc4oc5ccccc5c4c3-c3ccc4c(c3)sc3ccccc34)cc21] | 43.7 | 806.2 |
| | IM-A7 | CAS:932731-03-8 | 346 | | |
| Synthesis Example 40 | 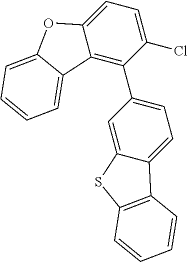 [SMILES: Clc1ccc2oc3ccccc3c2c1-c1ccc2c(c1)sc1ccccc12] | 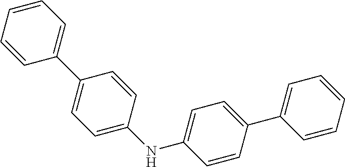 [SMILES: c1ccc(-c2ccc(Nc3ccc(-c4ccccc4)cc3)cc2)cc1] | 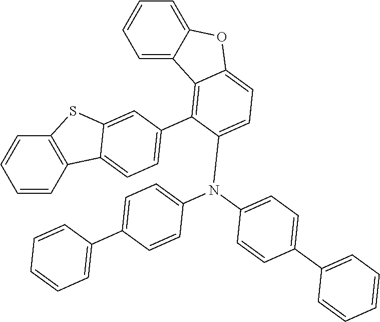 [SMILES: c1ccc(-c2ccc(N(c3ccc(-c4ccccc4)cc3)c3ccc4oc5ccccc5c4c3-c3ccc4c(c3)sc3ccccc34)cc2)cc1] | 41.0 | 670.2 |
| | IM-A7 | CAS:102113-98-4 | 351 | | |
| Synthesis Example 41 | 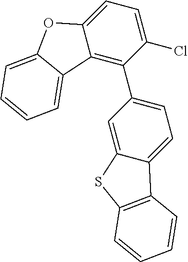 [SMILES: Clc1ccc2oc3ccccc3c2c1-c1ccc2c(c1)sc1ccccc12] | 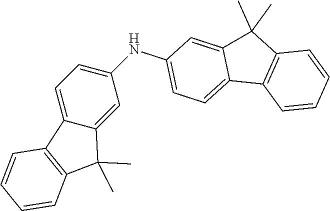 [SMILES: CC1(C)c2ccccc2-c2ccc(Nc3ccc4c(c3)C(C)(C)c3ccccc3-4)cc21] | 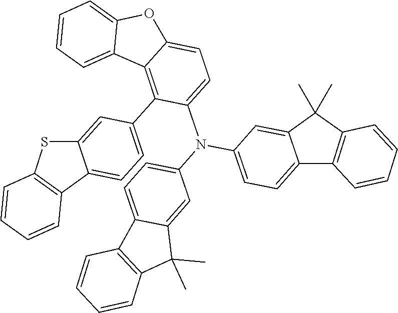 [SMILES: CC1(C)c2ccccc2-c2ccc(N(c3ccc4c(c3)C(C)(C)c3ccccc3-4)c3ccc4oc5ccccc5c4c3-c3ccc4c(c3)sc3ccccc34)cc21] | 36.7 | 750.3 |
| | IM-A7 | CAS:500717-23-7 | 371 | | |
| Synthesis Example 42 | 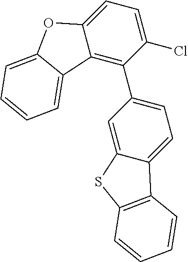 [SMILES: Clc1ccc2oc3ccccc3c2c1-c1ccc2c(c1)sc1ccccc12] | 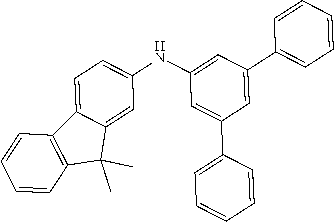 [SMILES: CC1(C)c2ccccc2-c2ccc(Nc3cc(-c4ccccc4)cc(-c4ccccc4)c3)cc21] | 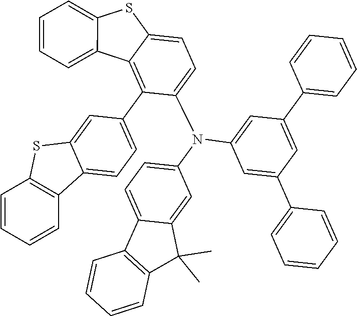 [SMILES: CC1(C)c2ccccc2-c2ccc(N(c3cc(-c4ccccc4)cc(-c4ccccc4)c3)c3ccc4sc5ccccc5c4c3-c3ccc4c(c3)sc3ccccc34)cc21] | 44.9 | 786.3 |
| | IM-A7 | CAS:1372778-68-1 | 380 | | |
| Synthesis Example 43 | 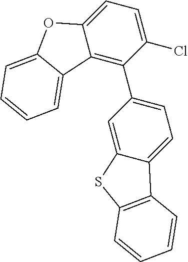 [SMILES: Clc1ccc2oc3ccccc3c2c1-c1ccc2c(c1)sc1ccccc12] | 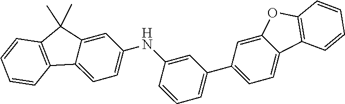 [SMILES: CC1(C)c2ccccc2-c2ccc(Nc3cccc(-c4ccc5c(c4)oc4ccccc45)c3)cc21] | 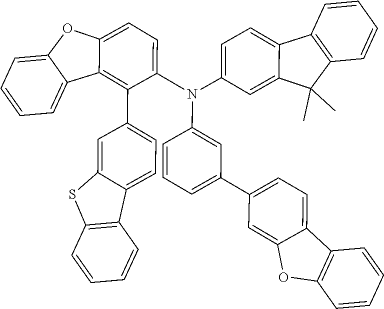 [SMILES: CC1(C)c2ccccc2-c2ccc(N(c3cccc(-c4ccc5c(c4)oc4ccccc45)c3)c3ccc4oc5ccccc5c4c3-c3ccc4c(c3)sc3ccccc34)cc21] | 46.1 | 800.3 |
| | IM-A7 | IM-N7 | 423 | | |
| Synthesis Example 44 | 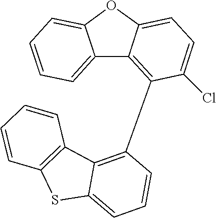 [SMILES: Clc1ccc2oc3ccccc3c2c1-c1cccc2sc3ccccc3c12] | 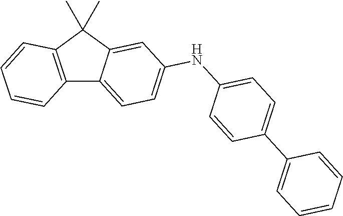 [SMILES: CC1(C)c2ccccc2-c2ccc(Nc3ccc(-c4ccccc4)cc3)cc21] | 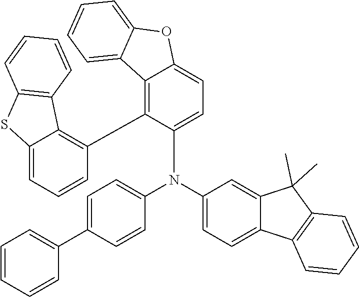 [SMILES: CC1(C)c2ccccc2-c2ccc(N(c3ccc(-c4ccccc4)cc3)c3ccc4oc5ccccc5c4c3-c3cccc4sc5ccccc5c34)cc21] | 40.3 | 710.2 |
| | IM-A8 | CAS:897671-69-1 | 434 | | |
| Synthesis Example 45 | 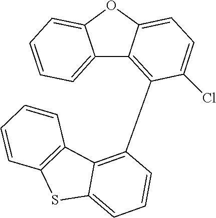 [SMILES: Clc1ccc2oc3ccccc3c2c1-c1cccc2sc3ccccc3c12] | 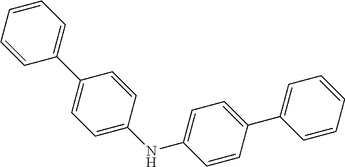 [SMILES: c1ccc(-c2ccc(Nc3ccc(-c4ccccc4)cc3)cc2)cc1] | 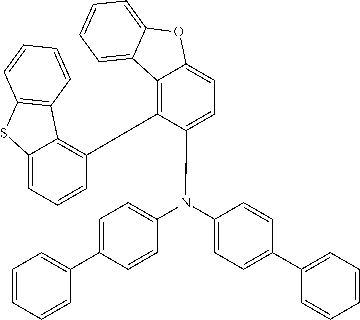 [SMILES: c1ccc(-c2ccc(N(c3ccc(-c4ccccc4)cc3)c3ccc4oc5ccccc5c4c3-c3cccc4sc5ccccc5c34)cc2)cc1] | 42.7 | 670.2 |
| | IM-A8 | CAS:102113-98-4 | 436 | | |
| Synthesis Example 46 | 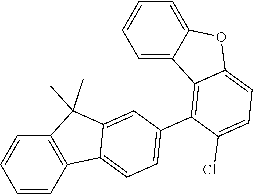 [SMILES: CC1(C)c2ccccc2-c2ccc(-c3c(Cl)ccc4oc5ccccc5c34)cc21] | 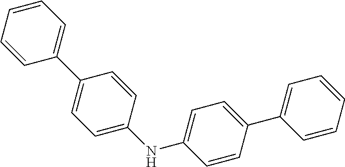 [SMILES: c1ccc(-c2ccc(Nc3ccc(-c4ccccc4)cc3)cc2)cc1] | 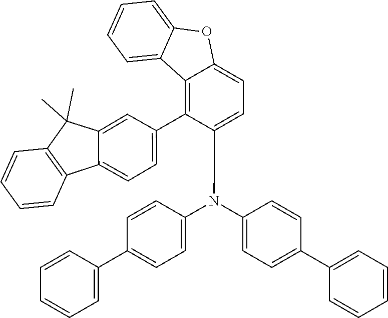 [SMILES: CC1(C)c2ccccc2-c2ccc(-c3c(N(c4ccc(-c5ccccc5)cc4)c4ccc(-c5ccccc5)cc4)ccc4oc5ccccc5c34)cc21] | 39.7 | 680.3 |
| | IM-A9 | CAS:102113-98-4 | 466 | | |
| Synthesis Example 47 | 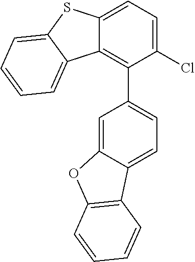 [SMILES: Clc1ccc2sc3ccccc3c2c1-c1ccc2c(c1)oc1ccccc12] | 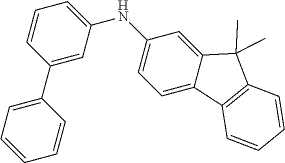 [SMILES: CC1(C)c2ccccc2-c2ccc(Nc3cccc(-c4ccccc4)c3)cc21] | 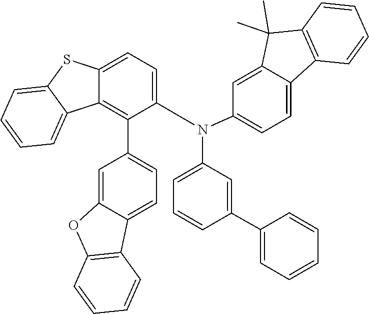 [SMILES: CC1(C)c2ccccc2-c2ccc(N(c3cccc(-c4ccccc4)c3)c3ccc4sc5ccccc5c4c3-c3ccc4c(c3)oc3ccccc34)cc21] | 40.5 | 710.2 |
| | IM-A10 | CAS:1372778-66-9 | 471 | | |
| Synthesis Example 48 | 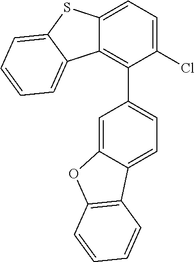 [SMILES: Clc1ccc2sc3ccccc3c2c1-c1ccc2c(c1)oc1ccccc12] | 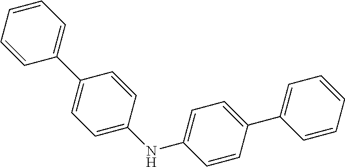 [SMILES: c1ccc(-c2ccc(Nc3ccc(-c4ccccc4)cc3)cc2)cc1] | 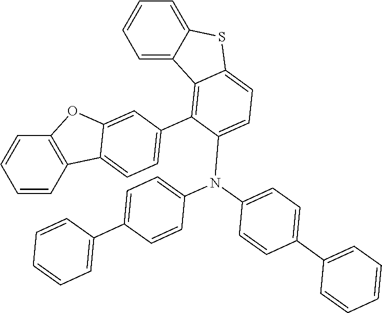 [SMILES: c1ccc(-c2ccc(N(c3ccc(-c4ccccc4)cc3)c3ccc4sc5ccccc5c4c3-c3ccc4c(c3)oc3ccccc34)cc2)cc1] | 49.0 | 670.2 |
| | IM-A10 | CAS:102113-98-4 | 476 | | |
| Synthesis Example 49 | 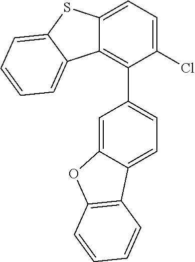 [SMILES: Clc1ccc2sc3ccccc3c2c1-c1ccc2c(c1)oc1ccccc12] | 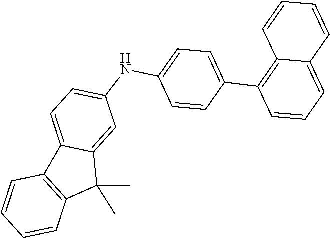 [SMILES: CC1(C)c2ccccc2-c2ccc(Nc3ccc(-c4cccc5ccccc45)cc3)cc21] | 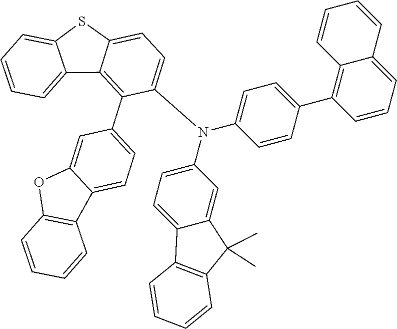 [SMILES: CC1(C)c2ccccc2-c2ccc(N(c3ccc(-c4cccc5ccccc45)cc3)c3ccc4sc5ccccc5c4c3-c3ccc4c(c3)oc3ccccc34)cc21] | 38.1 | 760.3 |
| | IM-A10 | CAS:1438387-72-4 | 490 | | |
| Synthesis Example 50 | 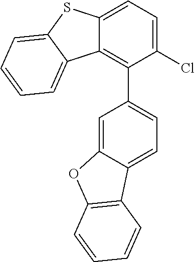 [SMILES: Clc1ccc2sc3ccccc3c2c1-c1ccc2c(c1)oc1ccccc12] | 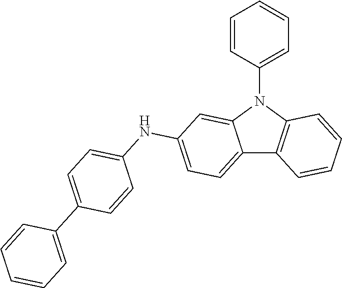 [SMILES: c1ccc(-c2ccc(Nc3ccc4c5ccccc5n(-c5ccccc5)c4c3)cc2)cc1] | 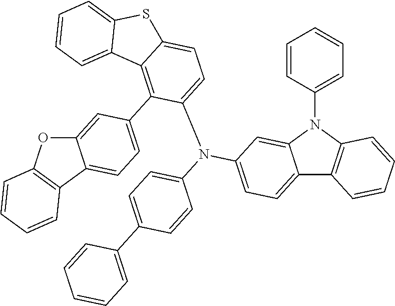 [SMILES: c1ccc(-c2ccc(N(c3ccc4c5ccccc5n(-c5ccccc5)c4c3)c3ccc4sc5ccccc5c4c3-c3ccc4c(c3)oc3ccccc34)cc2)cc1] | 45.6 | 759.2 |
| | IM-A10 | CAS:1427316-58-2 | 507 | | |
| Synthesis Example 51 | 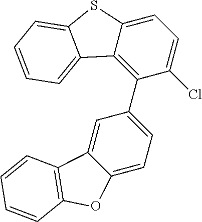 [SMILES: Clc1ccc2sc3ccccc3c2c1-c1ccc2oc3ccccc3c2c1] | 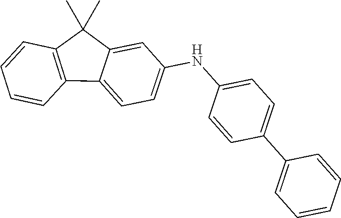 [SMILES: CC1(C)c2ccccc2-c2ccc(Nc3ccc(-c4ccccc4)cc3)cc21] | 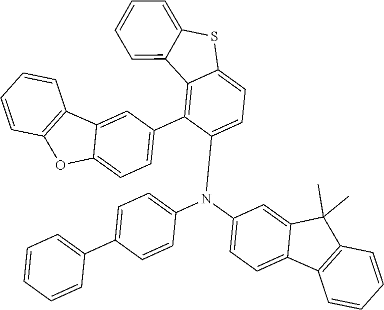 [SMILES: CC1(C)c2ccccc2-c2ccc(N(c3ccc(-c4ccccc4)cc3)c3ccc4sc5ccccc5c4c3-c3ccc4oc5ccccc5c4c3)cc21] | 49.0 | 710.2 |
| | IM-A11 | CAS:897671-69-1 | 534 | | |
| Synthesis Example 52 | 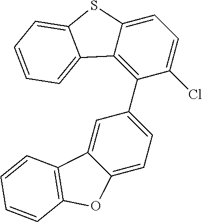 [SMILES: Clc1ccc2sc3ccccc3c2c1-c1ccc2oc3ccccc3c2c1] | 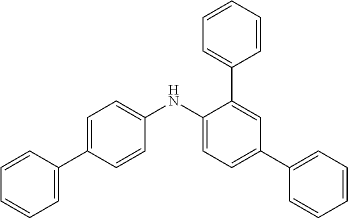 [SMILES: c1ccc(-c2ccc(Nc3ccc(-c4ccccc4)cc3-c3ccccc3)cc2)cc1] | 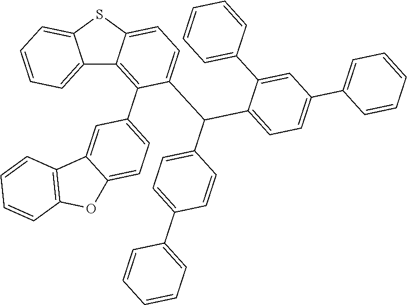 [SMILES: c1ccc(-c2ccc(C(c3ccc(-c4ccccc4)cc3-c3ccccc3)c3ccc4sc5ccccc5c4c3-c3ccc4oc5ccccc5c4c3)cc2)cc1] | 38.9 | 746.2 |
| | IM-A11 | CAS:1547491-85-9 | 549 | | |
| Synthesis Example 53 | 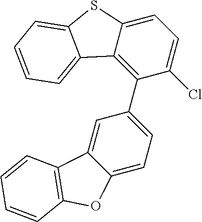 [SMILES: Clc1ccc2sc3ccccc3c2c1-c1ccc2oc3ccccc3c2c1] | 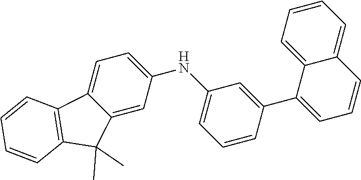 [SMILES: CC1(C)c2ccccc2-c2ccc(Nc3cccc(-c4cccc5ccccc45)c3)cc21] | 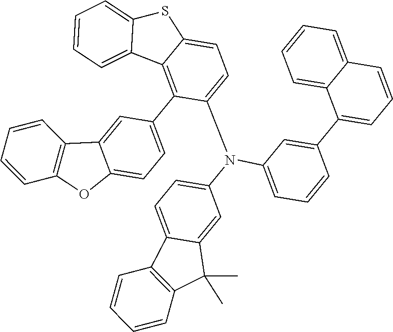 [SMILES: CC1(C)c2ccccc2-c2ccc(N(c3cccc(-c4cccc5ccccc45)c3)c3ccc4sc5ccccc5c4c3-c3ccc4oc5ccccc5c4c3)cc21] | 43.1 | 760.3 |
| | IM-A11 | IM-N9 | 557 | | |
| Synthesis Example 54 | 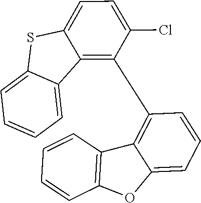 [SMILES: Clc1ccc2sc3ccccc3c2c1-c1cccc2oc3ccccc3c12] | 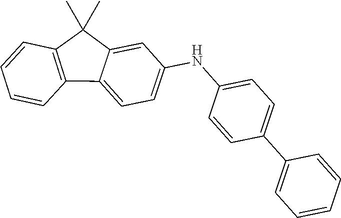 [SMILES: CC1(C)c2ccccc2-c2ccc(Nc3ccc(-c4ccccc4)cc3)cc21] | 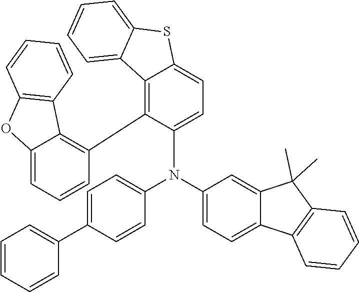 [SMILES: CC1(C)c2ccccc2-c2ccc(N(c3ccc(-c4ccccc4)cc3)c3ccc4sc5ccccc5c4c3-c3cccc4oc5ccccc5c34)cc21] | 38.8 | 710.2 |
| | IM-A12 | CAS:897671-69-1 | 609 | | |
| Synthesis Example 55 | 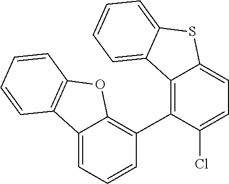 [SMILES: Clc1ccc2sc3ccccc3c2c1-c1cccc2c1oc1ccccc12] | 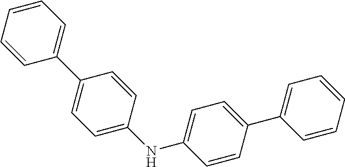 [SMILES: c1ccc(-c2ccc(Nc3ccc(-c4ccccc4)cc3)cc2)cc1] | 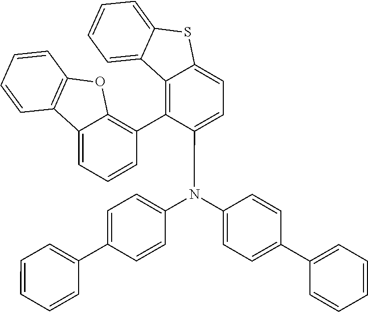 [SMILES: c1ccc(-c2ccc(N(c3ccc(-c4ccccc4)cc3)c3ccc4sc5ccccc5c4c3-c3cccc4c3oc3ccccc34)cc2)cc1] | 37.6 | 670.2 |
| | IM-A13 | CAS:102113-98-4 | 641 | | |
| Synthesis Example 56 | 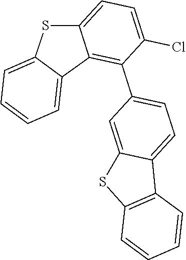 [SMILES: Clc1ccc2sc3ccccc3c2c1-c1ccc2c(c1)sc1ccccc12] | 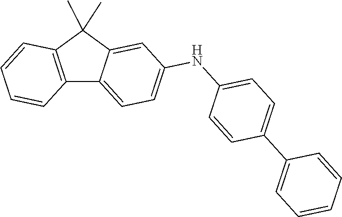 [SMILES: CC1(C)c2ccccc2-c2ccc(Nc3ccc(-c4ccccc4)cc3)cc21] | 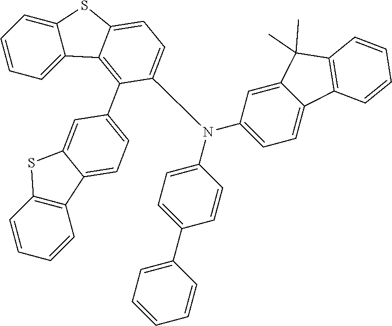 [SMILES: CC1(C)c2ccccc2-c2ccc(N(c3ccc(-c4ccccc4)cc3)c3ccc4sc5ccccc5c4c3-c3ccc4c(c3)sc3ccccc34)cc21] | 39.6 | 726.2 |
| | IM-A14 | CAS:897671-69-1 | 674 | | |
| Synthesis Example 57 | 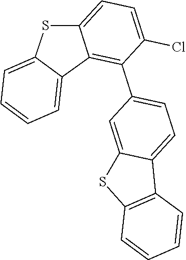 [SMILES: Clc1ccc2sc3ccccc3c2c1-c1ccc2c(c1)sc1ccccc12] | 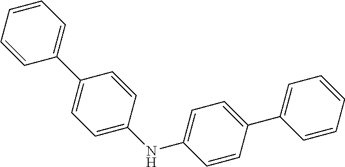 [SMILES: c1ccc(-c2ccc(Nc3ccc(-c4ccccc4)cc3)cc2)cc1] | 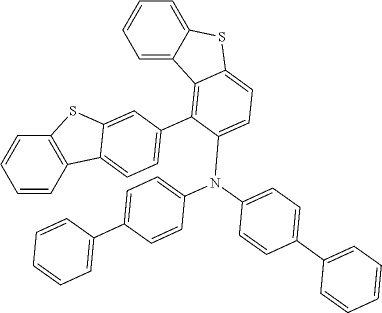 [SMILES: c1ccc(-c2ccc(N(c3ccc(-c4ccccc4)cc3)c3ccc4sc5ccccc5c4c3-c3ccc4c(c3)sc3ccccc34)cc2)cc1] | 48.2 | 686.2 |
| | IM-A14 | CAS:102113-98-4 | 681 | | |
| Synthesis Example 58 | 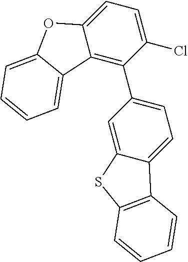 [SMILES: Clc1ccc2oc3ccccc3c2c1-c1ccc2c(c1)sc1ccccc12] | 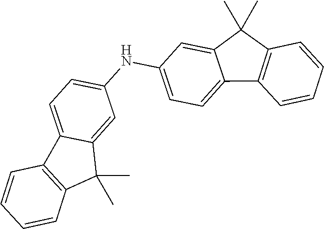 [SMILES: CC1(C)c2ccccc2-c2ccc(Nc3ccc4c(c3)C(C)(C)c3ccccc3-4)cc21] | 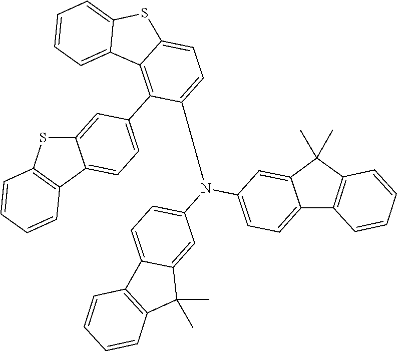 [SMILES: CC1(C)c2ccccc2-c2ccc(N(c3ccc4c(c3)C(C)(C)c3ccccc3-4)c3ccc4sc5ccccc5c4c3-c3ccc4c(c3)sc3ccccc34)cc21] | 41.8 | 766.3 |
| | IM-A14 | CAS:500717-23-7 | 691 | | |
| Synthesis Example 59 | 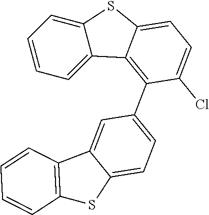 [SMILES: Clc1ccc2sc3ccccc3c2c1-c1ccc2sc3ccccc3c2c1] | 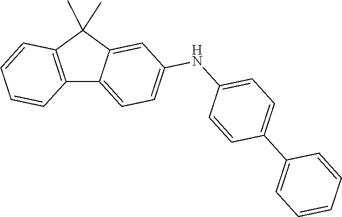 [SMILES: CC1(C)c2ccccc2-c2ccc(Nc3ccc(-c4ccccc4)cc3)cc21] | 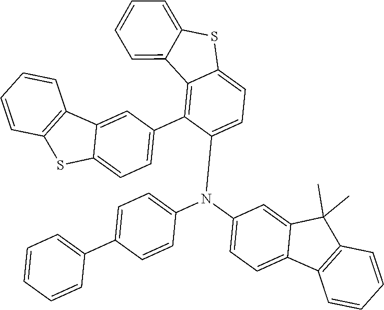 [SMILES: CC1(C)c2ccccc2-c2ccc(N(c3ccc(-c4ccccc4)cc3)c3ccc4sc5ccccc5c4c3-c3ccc4sc5ccccc5c4c3)cc21] | 49.6 | 726.2 |
| | IM-A2 | CAS:897671-69-1 | 704 | | |
| Synthesis Example 60 | 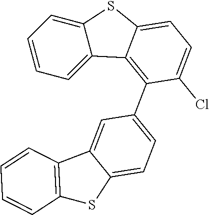 [SMILES: Clc1ccc2sc3ccccc3c2c1-c1ccc2sc3ccccc3c2c1] | 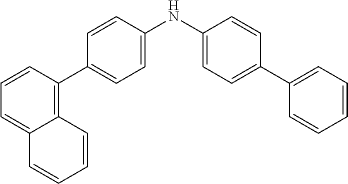 [SMILES: c1ccc(-c2ccc(Nc3ccc(-c4cccc5ccccc45)cc3)cc2)cc1] | 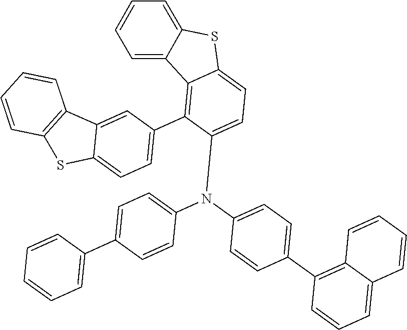 [SMILES: c1ccc(-c2ccc(N(c3ccc(-c4cccc5ccccc45)cc3)c3ccc4sc5ccccc5c4c3-c3ccc4sc5ccccc5c4c3)cc2)cc1] | 47.8 | 736.2 |
| | IM-A2 | CAS:897921-59-4 | 715 | | |
| Synthesis Example 61 | 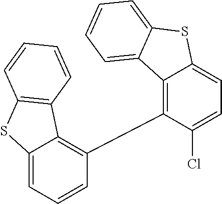 [SMILES: Clc1ccc2sc3ccccc3c2c1-c1cccc2sc3ccccc3c12] | 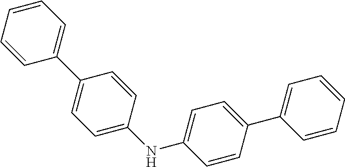 [SMILES: c1ccc(-c2ccc(Nc3ccc(-c4ccccc4)cc3)cc2)cc1] | 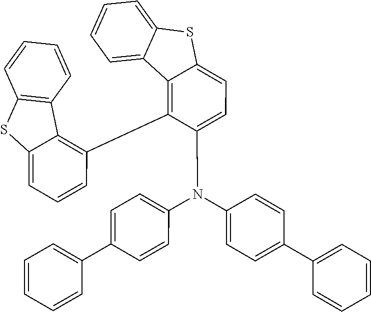 [SMILES: c1ccc(-c2ccc(N(c3ccc(-c4ccccc4)cc3)c3ccc4sc5ccccc5c4c3-c3cccc4sc5ccccc5c34)cc2)cc1] | 43.7 | 686.2 |
| | IM-A15 | CAS:102113-98-4 | 731 | | |
| Synthesis Example 62 | 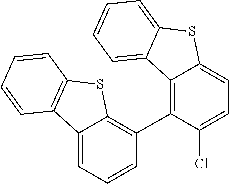 [SMILES: Clc1ccc2sc3ccccc3c2c1-c1cccc2c1sc1ccccc12] | 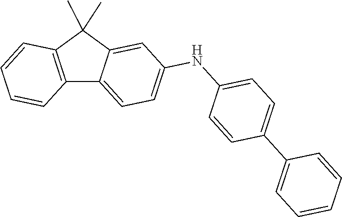 [SMILES: CC1(C)c2ccccc2-c2ccc(Nc3ccc(-c4ccccc4)cc3)cc21] | 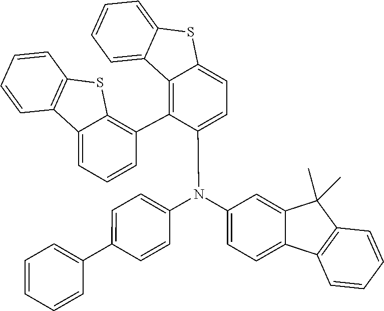 [SMILES: CC1(C)c2ccccc2-c2ccc(N(c3ccc(-c4ccccc4)cc3)c3ccc4sc5ccccc5c4c3-c3cccc4c3sc3ccccc34)cc21] | 39.8 | 726.2 |
| | IM-A16 | CAS:897671-69-1 | 764 | | |
| Synthesis Example 63 | 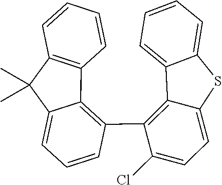 [SMILES: CC1(C)c2ccccc2-c2c(-c3c(Cl)ccc4sc5ccccc5c34)cccc21] | 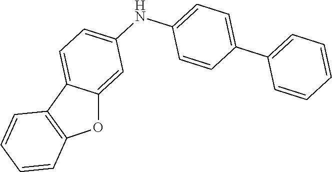 [SMILES: c1ccc(-c2ccc(Nc3ccc4c(c3)oc3ccccc34)cc2)cc1] | 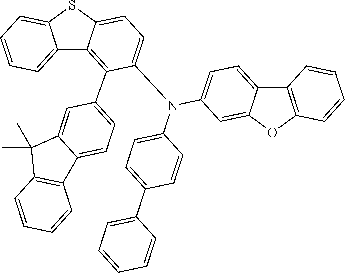 [SMILES: CC1(C)c2ccccc2-c2ccc(-c3c(N(c4ccc(-c5ccccc5)cc4)c4ccc5c(c4)oc4ccccc45)ccc4sc5ccccc5c34)cc21] | 38.6 | 710.2 |
| | IM-A17 | CAS:1290039-85-8 | 755 | | |
| Synthesis Example 64 | 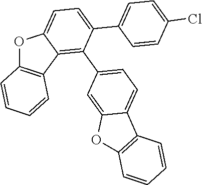 [SMILES: Clc1ccc(-c2ccc3oc4ccccc4c3c2-c2ccc3c(c2)oc2ccccc23)cc1] | 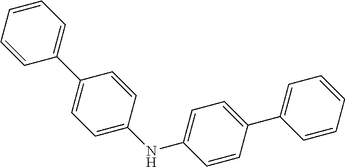 [SMILES: c1ccc(-c2ccc(Nc3ccc(-c4ccccc4)cc3)cc2)cc1] | 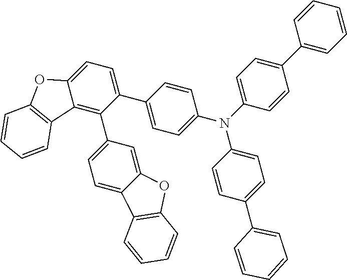 [SMILES: c1ccc(-c2ccc(N(c3ccc(-c4ccccc4)cc3)c3ccc(-c4ccc5oc6ccccc6c5c4-c4ccc5c(c4)oc4ccccc45)cc3)cc2)cc1] | 45.7 | 730.3 |
| | IM-A18 | CAS:102113-98-4 | 791 | | |
| Synthesis Example 65 | 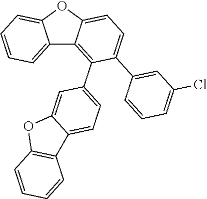 [SMILES: Clc1cccc(-c2ccc3oc4ccccc4c3c2-c2ccc3c(c2)oc2ccccc23)c1] | 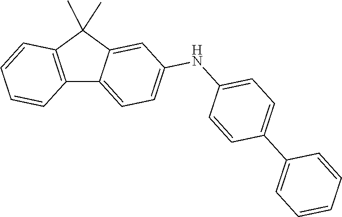 [SMILES: CC1(C)c2ccccc2-c2ccc(Nc3ccc(-c4ccccc4)cc3)cc21] | 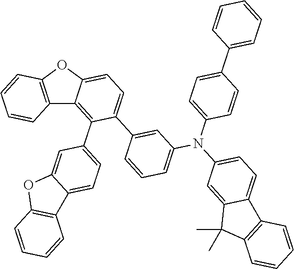 [SMILES: CC1(C)c2ccccc2-c2ccc(N(c3ccc(-c4ccccc4)cc3)c3cccc(-c4ccc5oc6ccccc6c5c4-c4ccc5c(c4)oc4ccccc45)c3)cc21] | 41.8 | 770.3 |
| | IM-A19 | CAS: 897671-69-1 | 798 | | |
| Synthesis Example 66 | 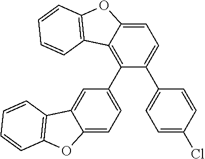 [SMILES: Clc1ccc(-c2ccc3oc4ccccc4c3c2-c2ccc3oc4ccccc4c3c2)cc1] | 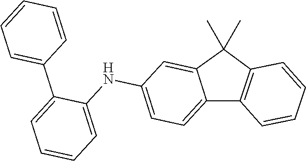 [SMILES: CC1(C)c2ccccc2-c2ccc(Nc3ccccc3-c3ccccc3)cc21] | 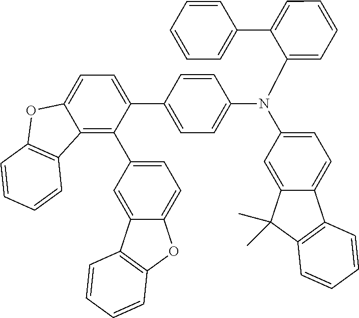 [SMILES: CC1(C)c2ccccc2-c2ccc(N(c3ccc(-c4ccc5oc6ccccc6c5c4-c4ccc5oc6ccccc6c5c4)cc3)c3ccccc3-c3ccccc3)cc21] | 40.9 | 770.3 |
| | IM-A20 | CAS:1198395-24-2 | 803 | | |
| Synthesis Example 67 | 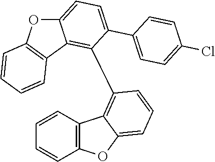 [SMILES: Clc1ccc(-c2ccc3oc4ccccc4c3c2-c2cccc3oc4ccccc4c23)cc1] | 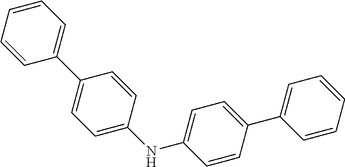 [SMILES: c1ccc(-c2ccc(Nc3ccc(-c4ccccc4)cc3)cc2)cc1] | 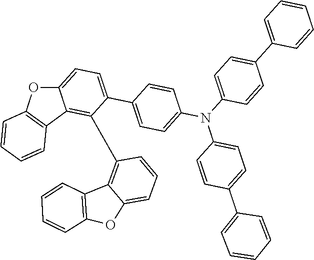 [SMILES: c1ccc(-c2ccc(N(c3ccc(-c4ccccc4)cc3)c3ccc(-c4ccc5oc6ccccc6c5c4-c4cccc5oc6ccccc6c45)cc3)cc2)cc1] | 45.5 | 730.3 |
| | IM-A21 | CAS:102113-98-4 | 811 | | |
| Synthesis Example 68 | 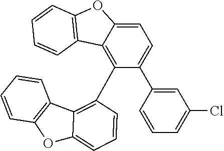 [SMILES: Clc1cccc(-c2ccc3oc4ccccc4c3c2-c2cccc3oc4ccccc4c23)c1] | 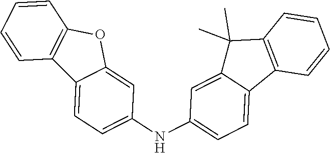 [SMILES: CC1(C)c2ccccc2-c2ccc(Nc3ccc4c(c3)oc3ccccc34)cc21] | 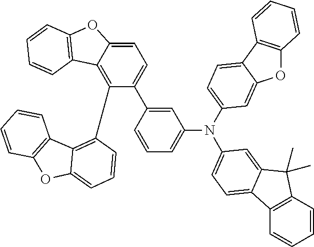 [SMILES: CC1(C)c2ccccc2-c2ccc(N(c3cccc(-c4ccc5oc6ccccc6c5c4-c4cccc5oc6ccccc6c45)c3)c3ccc4c(c3)oc3ccccc34)cc21] | 49.3 | 784.3 |
| | IM-A22 | CAS:1427556-50-0 | 820 | | |
| Synthesis Example 69 | 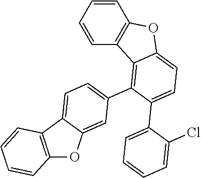 [SMILES: Clc1ccccc1-c1ccc2oc3ccccc3c2c1-c1ccc2c(c1)oc1ccccc12] | 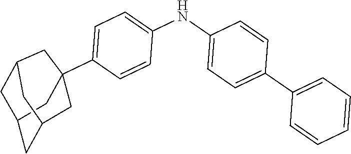 [SMILES: c1ccc(-c2ccc(Nc3ccc(C45CC6CC(CC(C6)C4)C5)cc3)cc2)cc1] | 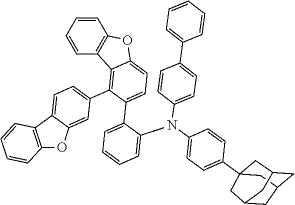 [SMILES: c1ccc(-c2ccc(N(c3ccc(C45CC6CC(CC(C6)C4)C5)cc3)c3ccccc3-c3ccc4oc5ccccc5c4c3-c3ccc4c(c3)oc3ccccc34)cc2)cc1] | 38.3 | 788.4 |
| | IM-A23 | IM-N10 | 833 | | |
| Synthesis Example 70 | 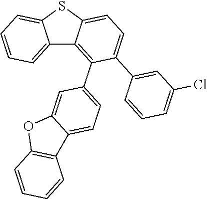 [SMILES: Clc1cccc(-c2ccc3sc4ccccc4c3c2-c2ccc3c(c2)oc2ccccc23)c1] | 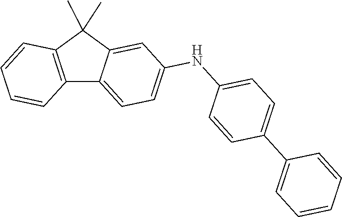 [SMILES: CC1(C)c2ccccc2-c2ccc(Nc3ccc(-c4ccccc4)cc3)cc21] | 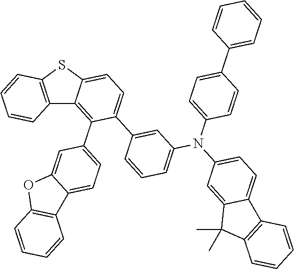 [SMILES: CC1(C)c2ccccc2-c2ccc(N(c3ccc(-c4ccccc4)cc3)c3cccc(-c4ccc5sc6ccccc6c5c4-c4ccc5c(c4)oc4ccccc45)c3)cc21] | 41.9 | 786.3 |
| | IM-A24 | CAS:897671-69-1 | 858 | | |
| Synthesis Example 71 | 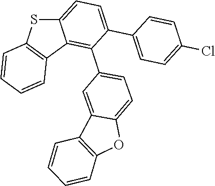 [SMILES: Clc1ccc(-c2ccc3sc4ccccc4c3c2-c2ccc3oc4ccccc4c3c2)cc1] | 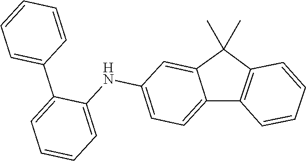 [SMILES: CC1(C)c2ccccc2-c2ccc(Nc3ccccc3-c3ccccc3)cc21] | 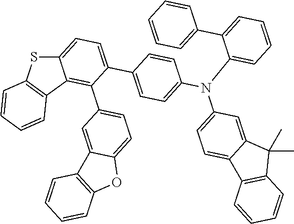 [SMILES: CC1(C)c2ccccc2-c2ccc(N(c3ccc(-c4ccc5sc6ccccc6c5c4-c4ccc5oc6ccccc6c5c4)cc3)c3ccccc3-c3ccccc3)cc21] | 43.2 | 786.3 |
| | IM-A25 | CAS:1198395-24-2 | 863 | | |
| Synthesis Example 72 | 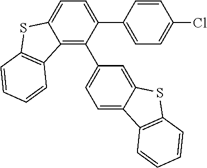 [SMILES: Clc1ccc(-c2ccc3sc4ccccc4c3c2-c2ccc3c(c2)sc2ccccc23)cc1] | 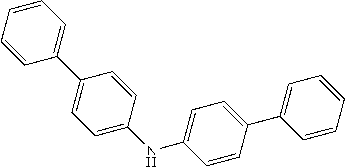 [SMILES: c1ccc(-c2ccc(Nc3ccc(-c4ccccc4)cc3)cc2)cc1] | 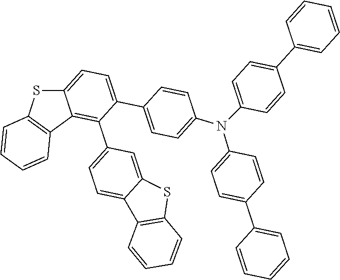 [SMILES: c1ccc(-c2ccc(N(c3ccc(-c4ccccc4)cc3)c3ccc(-c4ccc5sc6ccccc6c5c4-c4ccc5c(c4)sc4ccccc45)cc3)cc2)cc1] | 45.2 | 762.2 |
| | IM-A26 | CAS: 102113-98-4 | 896 | | |
| Synthesis Example 73 | 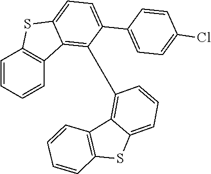 [SMILES: Clc1ccc(-c2ccc3sc4ccccc4c3c2-c2cccc3sc4ccccc4c23)cc1] | 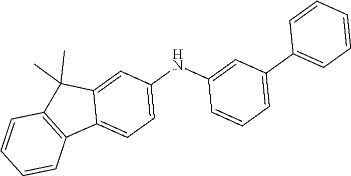 [SMILES: CC1(C)c2ccccc2-c2ccc(Nc3cccc(-c4ccccc4)c3)cc21] | 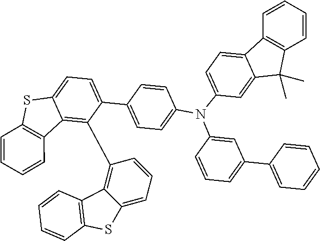 [SMILES: CC1(C)c2ccccc2-c2ccc(N(c3ccc(-c4ccc5sc6ccccc6c5c4-c4cccc5sc6ccccc6c45)cc3)c3cccc(-c4ccccc4)c3)cc21] | 44.6 | 802.3 |
| | IM-A27 | CAS:1372778-66-9 | 904 | | |

### NMR Data of Some Compounds:

NMR data of Compound 8: ¹H-NMR (400 MHz, Methylene-Chloride-D2) δ ppm 8.11 (d, 1H), 8.06 (d, 1H), 7.97 (d, 1H), 7.86 (d, 1H), 7.72 (d, 1H), 7.61-7.50 (m, 9H), 7.47-7.35 (m, 7H), 7.32 (d, 1H), 7.18-7.12 (m, 2H), 6.92 (d, 2H), 6.89 (d, 1H), 6.77 (d, 1H), 6.59 (s, 1H), 1.35 (s, 6H);

NMR data of Compound 715: ¹H-NMR (400 MHz, Methylene-Chloride-D2) δ ppm 8.28 (d, 1H), 8.20 (d, 1H), 8.18 (d, 1H), 8.04-7.99 (m, 3H), 7.89 (d, 1H), 7.81-7.76 (m, 3H), 7.59-7.49 (m, 7H), 7.47 (d, 1H), 7.44-7.39 (m, 5H), 7.35-7.28 (m, 4H), 7.09 (d, 1H), 6.91 (d, 2H), 6.78 (d, 2H), 6.52 (d, 1H);

NMR data of Compound 803: ¹H-NMR (400 MHz, Methylene-Chloride-D2) δ ppm 8.06 (s, 1H), 7.99 (d, 1H), 7.88 (d, 1H), 7.84 (d, 1H), 7.58-7.51 (m, 5H), 7.48-7.34 (m, 11H), 7.29-7.20 (m, 4H), 7.17-7.08 (m, 4H), 6.92 (d, 1H), 6.87 (d, 2H), 6.76 (d, 1H), 6.57 (s, 1H), 1.46 (s, 6H).

### Fabrication and Evaluation of Organic Electroluminescent Device

### Fabrication of Red Organic Electroluminescent Device

### Example 1

First, an anode pretreatment was performed by the following processes: the surface treatment of ITO/Ag/ITO substrate with a thickness of 100 Å, 1000 Å, and 100 Å in sequence was performed using ultraviolet ozone and O₂:N₂ plasma to increase the work function of the anode. The surface of the ITO substrate can also be cleaned with an organic solvent to remove impurities and oil stains thereon.

Compounds PD-01 and HT-01 were deposited by vacuum evaporation on the experimental substrate (anode) to form a hole injection layer (HIL) with a thickness of 110 Å, then compound HT-01 was deposited by vacuum evaporation on the hole injection layer to form a hole transport layer (HTL) with a thickness of 1200 Å.

Compound 8 was deposited by vacuum evaporation on the hole transport layer to form a hole auxiliary layer (also referred to a hole adjustment layer) with a thickness of 85 Å. Then, on the hole adjustment layer, compound RH-01 and compound RD-01 were co-deposited at a deposition rate ratio of 95%:5% to form a red light-emitting layer (EML) with a thickness of 320 Å.

Compound HB-01 was deposited by vacuum evaporation on the light-emitting layer to form a hole blocking layer (HBL) with a thickness of 500 Å. Then, on the hole blocking layer, compound ET-01 and LiQ were mixed at a weight ratio of 1:1 and deposited to form an electron transport layer (ETL) with a thickness of 300 Å. Yb was deposited on the electron transport layer to form an electron injection layer (EIL) with a thickness of 15 Å. Then, magnesium (Mg) and silver (Ag) were co-deposited on the electron injection layer at a deposition rate of 1:9 to form a cathode with a thickness of 10 Å.

In addition, compound CP-01 was deposited by vacuum evaporation on the cathode as a cathode protective layer (CPL) with a thickness of 630 Å, thereby completing the fabrication of an organic electroluminescent device.

### Example 2 to Example 73

Organic electroluminescent devices were fabricated by the same method as Example 1, except that the compounds in the Table 4 were used instead of compound 8 in Example 1, when the hold auxiliary layer was formed.

### Comparative Example 1 to Comparative Example 4

Organic electroluminescent devices were fabricated by the same method as used in Example 1, except that compound A to compound D were used instead of compound 8 in Example 1, when the hold auxiliary layer was formed.

In the preparation of organic electroluminescent devices, the structures of the materials used in the comparative examples and the examples are as follows:

The main material structures used in the above examples and comparative examples are as follows:

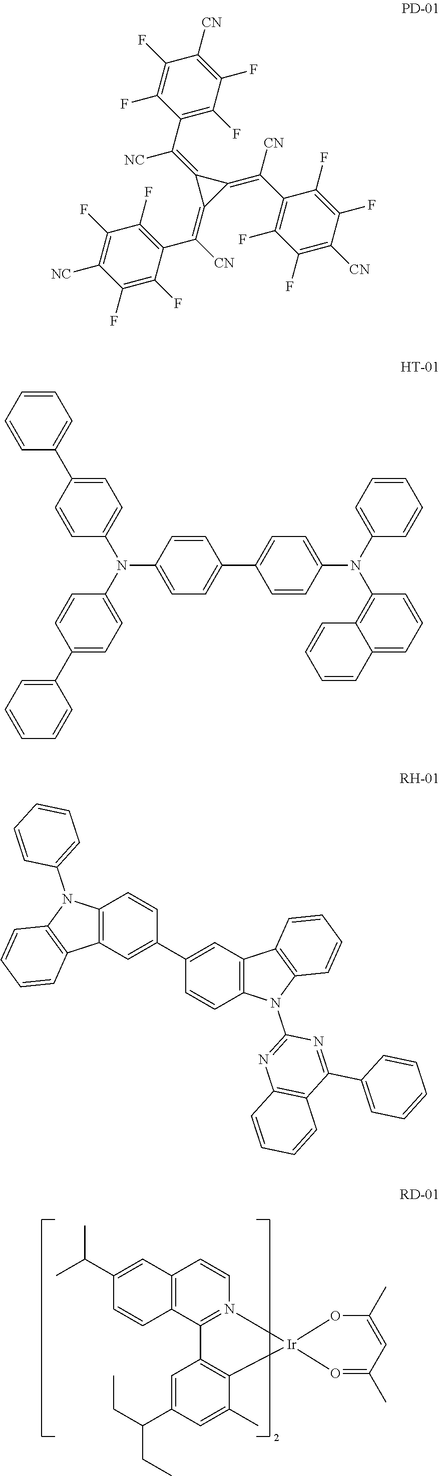

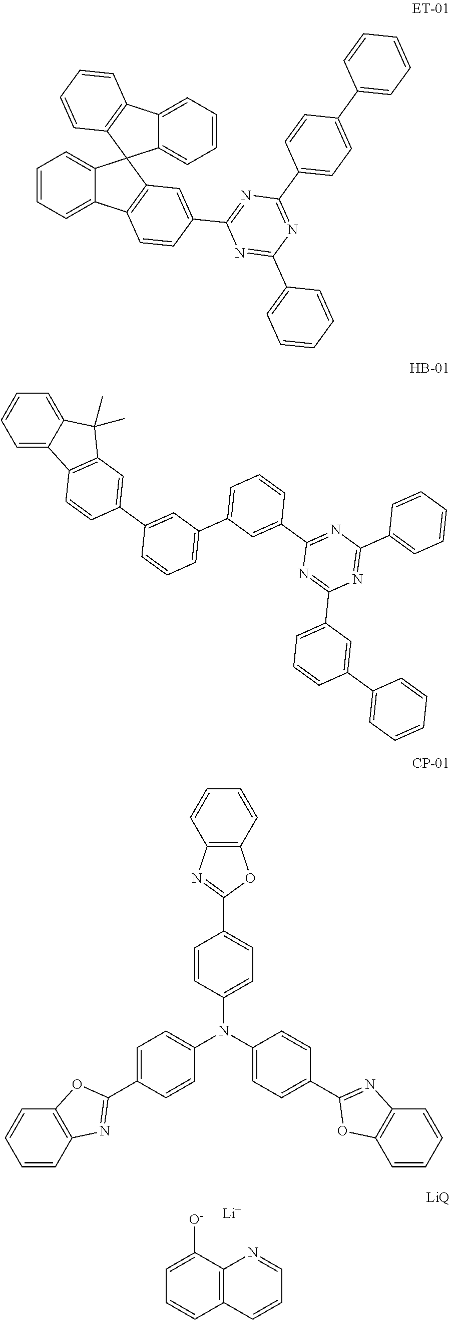 [SMILES: c1ccc(-c2ccc(-c3nc(-c4ccccc4)nc(-c4ccc5c(c4)C4(c6ccccc6-c6ccccc64)c4ccccc4-5)n3)cc2)cc1 ; CC1(C)c2ccccc2-c2ccc(-c3cccc(-c4cccc(-c5nc(-c6ccccc6)nc(-c6cccc(-c7ccccc7)c6)n5)c4)c3)cc21 ; c1ccc2oc(-c3ccc(N(c4ccc(-c5nc6ccccc6o5)cc4)c4ccc(-c5nc6ccccc6o5)cc4)cc3)nc2c1 ; [Li+] ; [O-]c1cccc2cccnc12]

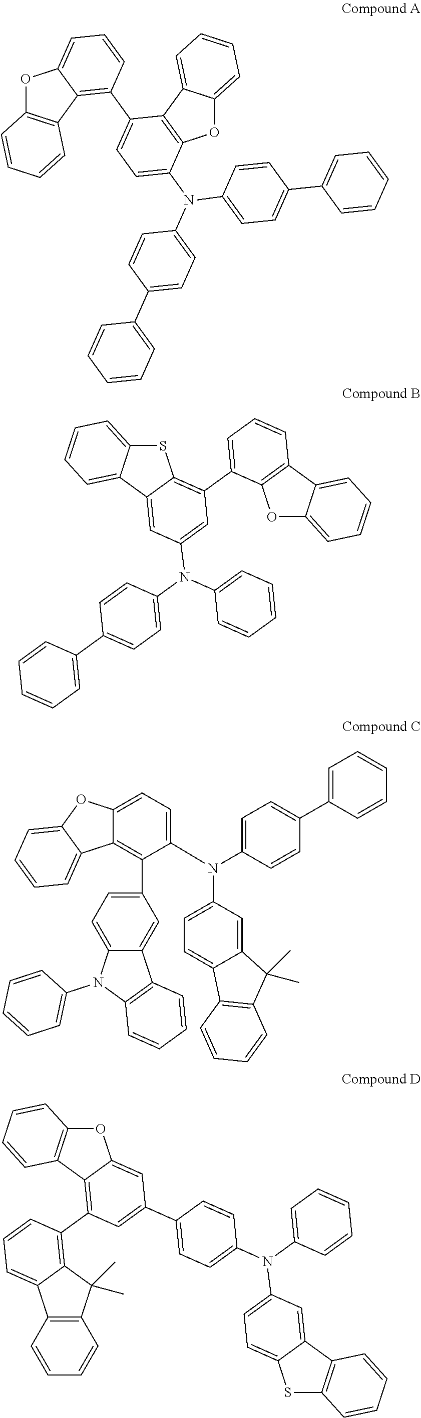

The performance of the organic electroluminescent devices prepared in Examples 1 to 73 and Comparative Examples 1 to 4 was tested. Specifically, the IVL performance of the devices was tested under the condition of 10 mA/cm², and the T95 device lifetime was tested under the condition of 20 mA/cm². The test results are shown in Table 5 below.

TABLE 5

| | | Oper- | | | | |
|---|---|---|---|---|---|---|
| | Hole | ating | Effi- | Color | | T95% |
| Example | Auxiliary | Voltage | ciency | Coordinates | | lifetime |
| No. | Layer | (V) | (Cd/A) | CIEx | CIEy | (hour) |
| Example 1 | Compound 8 | 3.92 | 61.3 | 0.68 | 0.32 | 364 |
| Example 2 | Compound 10 | 3.98 | 60.3 | 0.68 | 0.32 | 355 |
| Example 3 | Compound 19 | 3.96 | 61.0 | 0.68 | 0.32 | 359 |
| Example 4 | Compound 21 | 4.00 | 59.6 | 0.68 | 0.32 | 363 |
| Example 5 | Compound 25 | 3.99 | 61.4 | 0.68 | 0.32 | 370 |
| Example 6 | Compound 31 | 3.98 | 60.0 | 0.68 | 0.32 | 370 |
| Example 7 | Compound 40 | 4.03 | 59.2 | 0.68 | 0.32 | 366 |
| Example 8 | Compound 44 | 3.99 | 59.3 | 0.68 | 0.32 | 354 |
| Example 9 | Compound 48 | 3.97 | 60.4 | 0.68 | 0.32 | 359 |
| Example 10 | Compound 56 | 4.02 | 59.2 | 0.68 | 0.32 | 367 |
| Example 11 | Compound 64 | 3.88 | 59.4 | 0.68 | 0.32 | 356 |
| Example 12 | Compound 67 | 3.99 | 59.3 | 0.68 | 0.32 | 353 |
| Example 13 | Compound 71 | 3.92 | 60.8 | 0.68 | 0.32 | 367 |
| Example 14 | Compound 89 | 3.98 | 60.4 | 0.68 | 0.32 | 370 |
| Example 15 | Compound 93 | 3.90 | 59.9 | 0.68 | 0.32 | 369 |
| Example 16 | Compound 94 | 3.96 | 59.7 | 0.68 | 0.32 | 365 |
| Example 17 | Compound 95 | 4.03 | 59.3 | 0.68 | 0.32 | 355 |
| Example 18 | Compound 111 | 4.00 | 60.5 | 0.68 | 0.32 | 358 |
| Example 19 | Compound 116 | 3.96 | 59.9 | 0.68 | 0.32 | 362 |
| Example 20 | Compound 119 | 3.90 | 59.5 | 0.68 | 0.32 | 359 |
| Example 21 | Compound 123 | 3.89 | 57.2 | 0.68 | 0.32 | 364 |
| Example 22 | Compound 131 | 3.93 | 61.3 | 0.68 | 0.32 | 354 |
| Example 23 | Compound 140 | 3.92 | 60.1 | 0.68 | 0.32 | 355 |
| Example 24 | Compound 141 | 4.05 | 59.7 | 0.68 | 0.32 | 361 |
| Example 25 | Compound 143 | 3.95 | 59.0 | 0.68 | 0.32 | 357 |
| Example 26 | Compound 146 | 3.93 | 59.1 | 0.68 | 0.32 | 365 |
| Example 27 | Compound 155 | 3.90 | 59.9 | 0.68 | 0.32 | 371 |
| Example 28 | Compound 157 | 4.00 | 60.9 | 0.68 | 0.32 | 356 |
| Example 29 | Compound 181 | 3.90 | 59.1 | 0.68 | 0.32 | 360 |
| Example 30 | Compound 186 | 3.92 | 58.4 | 0.68 | 0.32 | 362 |
| Example 31 | Compound 219 | 3.90 | 61.6 | 0.68 | 0.32 | 362 |
| Example 32 | Compound 225 | 4.05 | 59.7 | 0.68 | 0.32 | 368 |
| Example 33 | Compound 251 | 3.89 | 61.4 | 0.68 | 0.32 | 363 |
| Example 34 | Compound 259 | 3.93 | 59.6 | 0.68 | 0.32 | 320 |
| Example 35 | Compound 265 | 4.01 | 60.6 | 0.68 | 0.32 | 316 |
| Example 36 | Compound 266 | 3.94 | 60.0 | 0.68 | 0.32 | 325 |
| Example 37 | Compound 312 | 4.01 | 59.6 | 0.68 | 0.32 | 319 |
| Example 38 | Compound 339 | 3.96 | 59.3 | 0.68 | 0.32 | 323 |
| Example 39 | Compound 346 | 3.89 | 60.9 | 0.68 | 0.32 | 314 |
| Example 40 | Compound 351 | 4.04 | 60.1 | 0.68 | 0.32 | 316 |
| Example 41 | Compound 371 | 3.88 | 61.0 | 0.68 | 0.32 | 322 |
| Example 42 | Compound 380 | 3.94 | 61.3 | 0.68 | 0.32 | 318 |
| Example 43 | Compound 423 | 4.00 | 59.7 | 0.68 | 0.32 | 312 |
| Example 44 | Compound 434 | 3.95 | 59.2 | 0.68 | 0.32 | 317 |
| Example 45 | Compound 436 | 3.98 | 61.4 | 0.68 | 0.32 | 321 |
| Example 46 | Compound 466 | 4.01 | 54.2 | 0.68 | 0.32 | 309 |
| Example 47 | Compound 471 | 3.96 | 59.3 | 0.68 | 0.32 | 374 |
| Example 48 | Compound 476 | 3.90 | 59.9 | 0.68 | 0.32 | 366 |
| Example 49 | Compound 490 | 3.98 | 59.3 | 0.68 | 0.32 | 375 |
| Example 50 | Compound 507 | 3.97 | 61.6 | 0.68 | 0.32 | 369 |
| Example 51 | Compound 534 | 3.96 | 58.9 | 0.68 | 0.32 | 359 |
| Example 52 | Compound 549 | 4.02 | 59.8 | 0.68 | 0.32 | 356 |
| Example 53 | Compound 557 | 3.95 | 59.1 | 0.68 | 0.32 | 370 |
| Example 54 | Compound 609 | 3.93 | 62.0 | 0.68 | 0.32 | 365 |
| Example 55 | Compound 641 | 3.92 | 60.5 | 0.68 | 0.32 | 371 |
| Example 56 | Compound 674 | 3.97 | 59.6 | 0.68 | 0.32 | 312 |
| Example 57 | Compound 681 | 4.00 | 60.3 | 0.68 | 0.32 | 314 |
| Example 58 | Compound 691 | 3.96 | 61.5 | 0.68 | 0.32 | 317 |
| Example 59 | Compound 704 | 4.00 | 59.2 | 0.68 | 0.32 | 318 |
| Example 60 | Compound 715 | 3.91 | 59.4 | 0.68 | 0.32 | 320 |
| Example 61 | Compound 731 | 3.88 | 59.7 | 0.68 | 0.32 | 315 |
| Example 62 | Compound 764 | 3.92 | 59.5 | 0.68 | 0.32 | 316 |
| Example 63 | Compound 755 | 3.98 | 53.8 | 0.68 | 0.32 | 305 |
| Example 64 | Compound 791 | 4.05 | 61.6 | 0.68 | 0.32 | 359 |
| Example 65 | Compound 798 | 4.09 | 62.0 | 0.68 | 0.32 | 362 |
| Example 66 | Compound 803 | 4.07 | 62.8 | 0.68 | 0.32 | 368 |
| Example 67 | Compound 811 | 4.08 | 60.4 | 0.68 | 0.32 | 364 |
| Example 68 | Compound 820 | 4.04 | 60.7 | 0.68 | 0.32 | 372 |
| Example 69 | Compound 833 | 4.04 | 61.5 | 0.68 | 0.32 | 379 |
| Example 70 | Compound 858 | 4.07 | 62.1 | 0.68 | 0.32 | 369 |
| Example 71 | Compound 863 | 4.09 | 63.8 | 0.68 | 0.32 | 373 |
| Example 72 | Compound 896 | 4.00 | 59.4 | 0.68 | 0.32 | 317 |
| Example 73 | Compound 904 | 4.04 | 61.1 | 0.68 | 0.32 | 320 |
| Comparative | Compound A | 4.28 | 47.3 | 0.68 | 0.32 | 233 |
| Example 1 | | | | | | |
| Comparative | Compound B | 4.33 | 45.4 | 0.68 | 0.32 | 225 |
| Example 2 | | | | | | |
| Comparative | Compound C | 4.24 | 48.5 | 0.68 | 0.32 | 258 |
| Example 3 | | | | | | |
| Comparative | Compound D | 4.31 | 46.0 | 0.68 | 0.32 | 243 |
| Example 4 | | | | | | |

Referring to Table 5 above, it can be seen that compared to the organic electroluminescent devices of Comparative Examples 1 to 4, the performance of the organic electroluminescent devices of Examples 1 to 73 has been greatly improved, mainly manifested in an increase in luminous efficiency of at least 10.9% and an increase in T95 lifetime of at least 18.2%.

The preferred embodiments of the present disclosure are described in detail above in conjunction with the accompanying drawings. However, the present disclosure is not limited to the specific details of the above embodiments. Within the scope of the technical concept of the present disclosure, various simple modifications can be made to the technical solutions of the present disclosure, and these simple modifications all fall within the protection scope of the present disclosure.

1. An arylamine compound, having a structure shown in Formula 1: 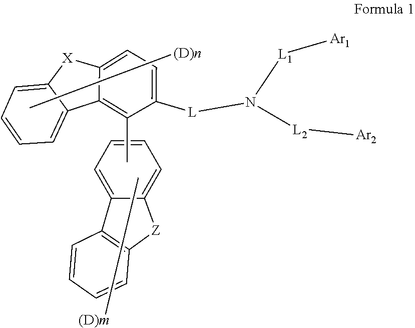
wherein X is selected from O and S; Z is selected from O, S, and C(R₁R₂);
R₁ and R₂ are the same or different, and are each independently selected from an alkyl having 1 to 4 carbon atoms and a deuteroalkyl having 1 to 4 carbon atoms;
m represents the number of D, and m is selected from 0, 1, 2, 3, 4, 5, 6, and 7;
n represents the number of D, and n is selected from 0, 1, 2, 3, 4, 5, and 6;
L is selected from a single bond and a substituted or unsubstituted phenylene;
L₁ and L₂ are the same or different, and are each independently selected from the group consisting of a single bond, a substituted or unsubstituted arylene having 6 to 15 carbon atoms, and a substituted or unsubstituted heteroarylene having 12 to 18 carbon atoms;
the substituent(s) in L, L₁, and L₂ are the same or different, and are each independently selected from the group consisting of a deuterium, a cyano, a halogen group, an alkyl having 1 to 4 carbon atoms, a haloalkyl having 1 to 4 carbon atoms, a deuteroalkyl having 1 to 4 carbon atoms, and a phenyl;
Ar₁ and Ar₂ are the same or different, and are each independently selected from a substituted or unsubstituted aryl having 6 to 40 carbon atoms and a substituted or unsubstituted heteroaryl having 5 to 40 carbon atoms;
the substituent(s) in Ar₁ and Ar₂ are the same or different, and are each independently selected from the group consisting of a deuterium, a cyano, a halogen group, an alkyl having 1 to 10 carbon atoms, a haloalkyl having 1 to 10 carbon atoms, a deuteroalkyl having 1 to 10 carbon atoms, an alkoxy having 1 to 10 carbon atoms, a trialkylsilyl having 3 to 12 carbon atoms, a triphenylsilyl, an aryl having 6 to 20 carbon atoms, a deuteroaryl having 6 to 20 carbon atoms, a heteroaryl having 3 to 20 carbon atoms, a cycloalkyl having 3 to 10 carbon atoms, an alkylthio having 1 to 10 carbon atoms, an aryloxy having 6 to 20 carbon atoms, and an arylthio having 6 to 20 carbon atoms; optionally, any two adjacent substituents form a 5- to 15-membered ring.

2. The arylamine compound according to claim 1, wherein L₁ and L₂ are the same or different, and are each independently selected from the group consisting of a single bond, a substituted or unsubstituted phenylene, a substituted or unsubstituted naphthylene, a substituted or unsubstituted biphenylene, a substituted or unsubstituted phenanthrylene, a substituted or unsubstituted dibenzothienylene, a substituted or unsubstituted dibenzofuranylene, and a substituted or unsubstituted carbazolylene;
optionally, the substituent(s) in L₁ and L₂ are the same or different, and are each independently selected from the group consisting of a deuterium, a fluoro, a cyano, a methyl, an ethyl, an isopropyl, a tert-butyl, a trifluoromethyl, a trideuteromethyl, and a phenyl.

3. The arylamine compound according to claim 1, wherein L is selected from a single bond and the following groups:

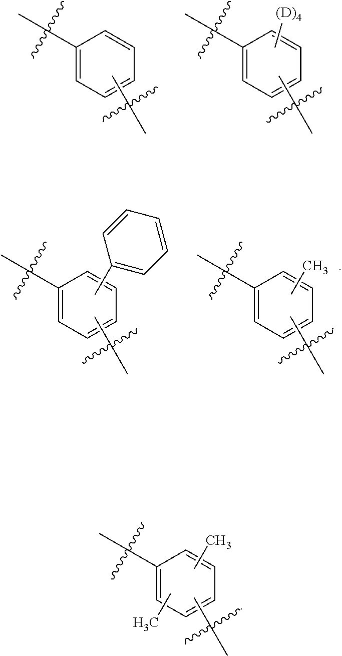

4. The arylamine compound according to claim 1, wherein L₁ and L₂ are each independently selected from a single bond and the group consisting of the following groups:

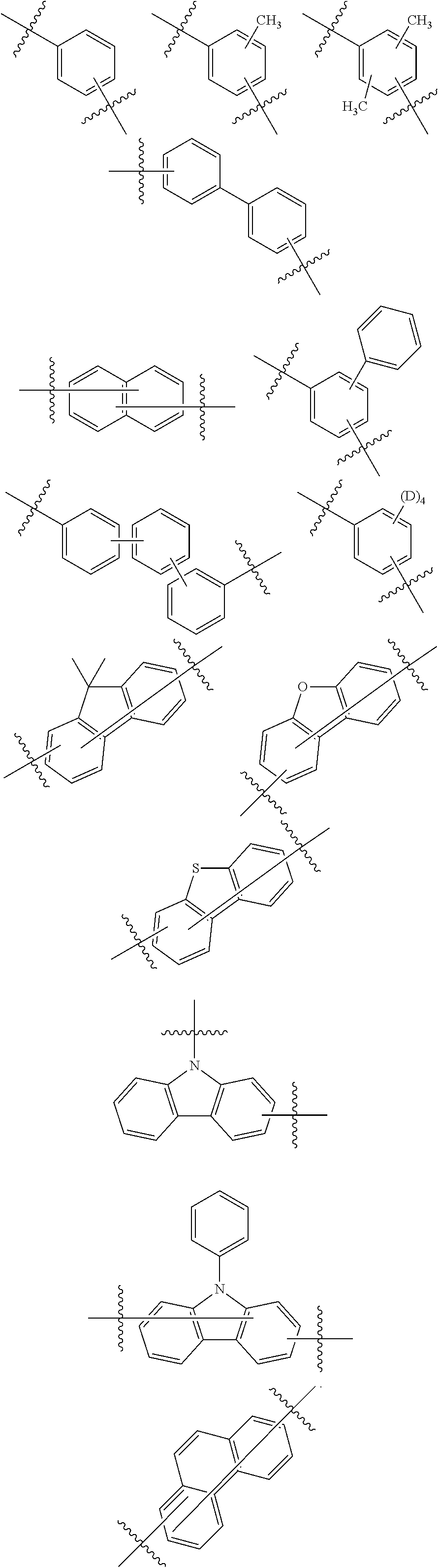

5. The arylamine compound according to claim 1, wherein Ar₁ and Ar₂ are each independently selected from the group consisting of a substituted or unsubstituted phenyl, a substituted or unsubstituted biphenyl, a substituted or unsubstituted terphenyl, a substituted or unsubstituted naphthyl, a substituted or unsubstituted anthryl, a substituted or unsubstituted phenanthryl, a substituted or unsubstituted fluorenyl, a substituted or unsubstituted spirodifluorenyl, a substituted or unsubstituted triphenylene, a substituted or unsubstituted pyrenyl, a substituted or unsubstituted perylenyl, a substituted or unsubstituted dibenzothienyl, a substituted or unsubstituted dibenzofuranyl, a substituted or unsubstituted carbazolyl, a substituted or unsubstituted benzothiazolyl, and a substituted or unsubstituted benzoxazolyl;
optionally, the substituent(s) in Ar₁ and Ar₂ are each independently selected from the group consisting of a deuterium, a fluoro, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a pyridyl, a dibenzofuranyl, a dibenzothienyl, and a carbazolyl; optionally, any two adjacent substituents in Ar₁ and Ar₂ form a benzene ring or a fluorene ring.

6. The arylamine compound according to claim 1, wherein Ar₁ and Ar₂ are each independently selected from the group consisting of the following groups:

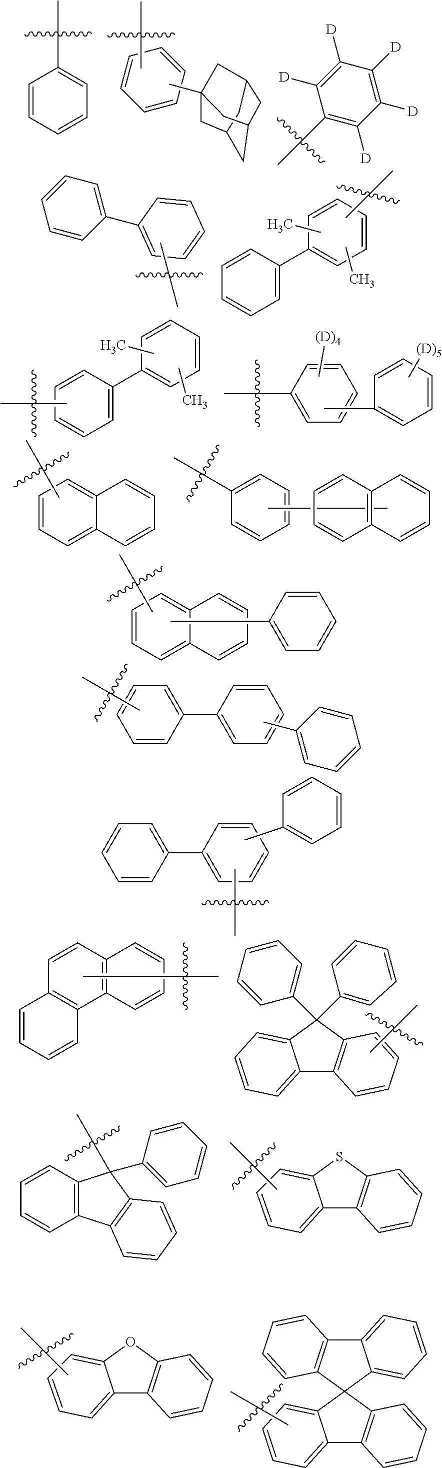

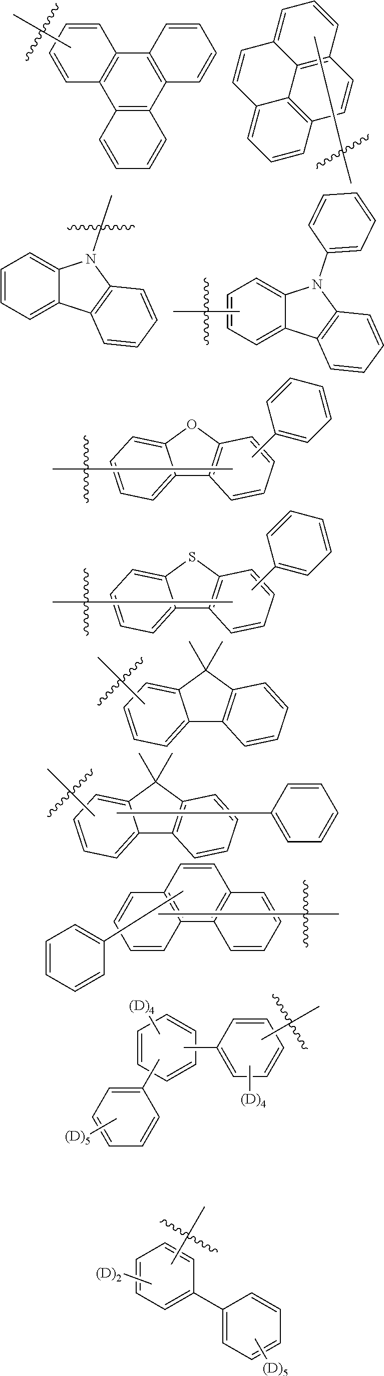

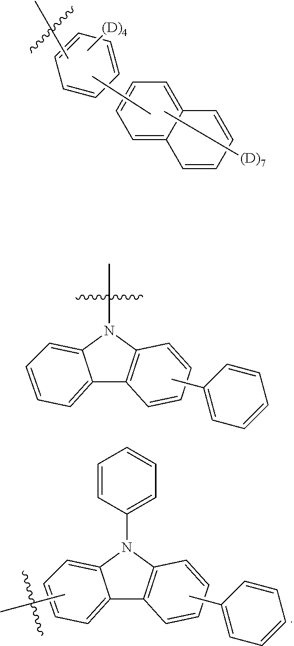

7. The arylamine compound according to claim 1, wherein and 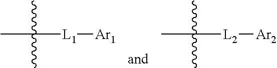
are each independently selected from the following groups:

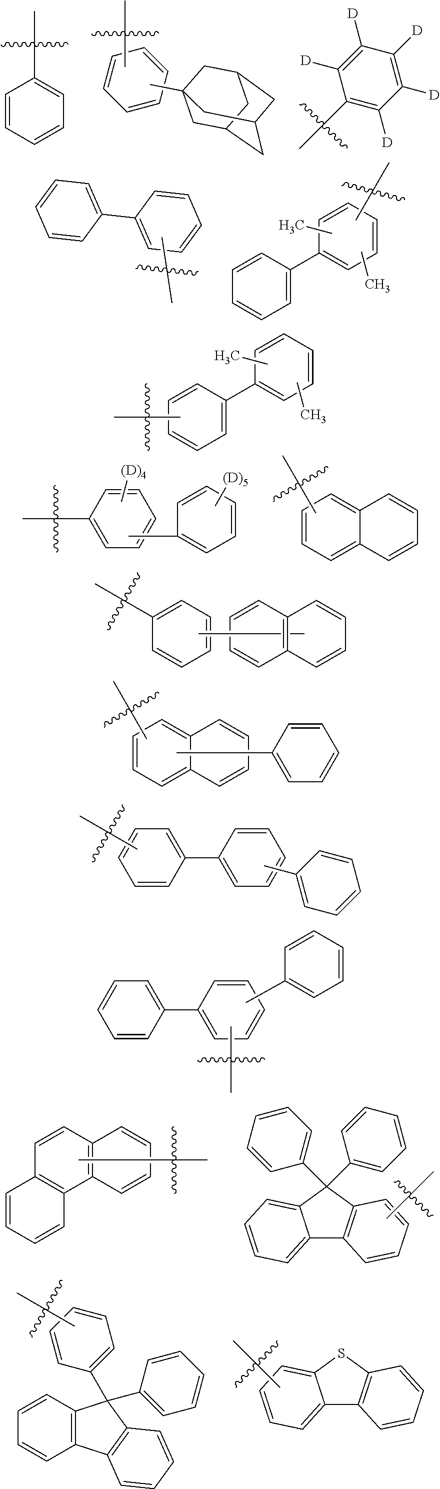

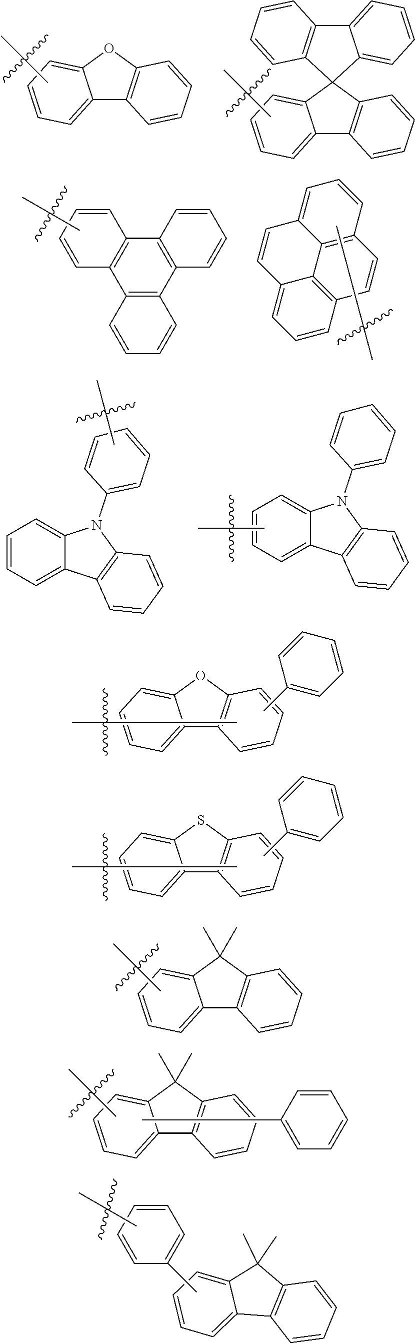

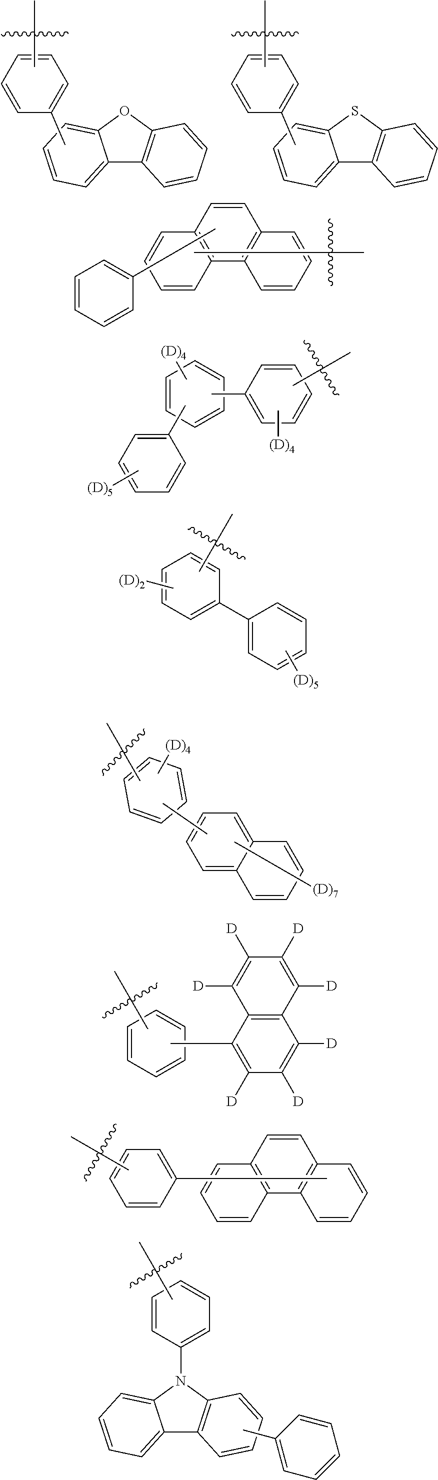

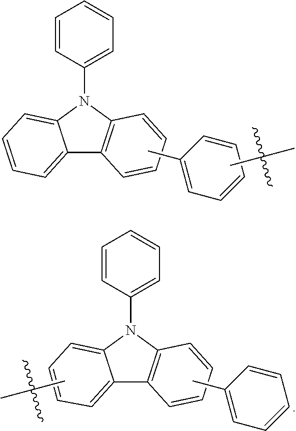

8. The arylamine compound according to claim 1, wherein 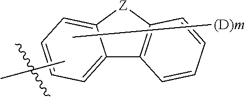
is in Formula 1 is selected from the following groups:

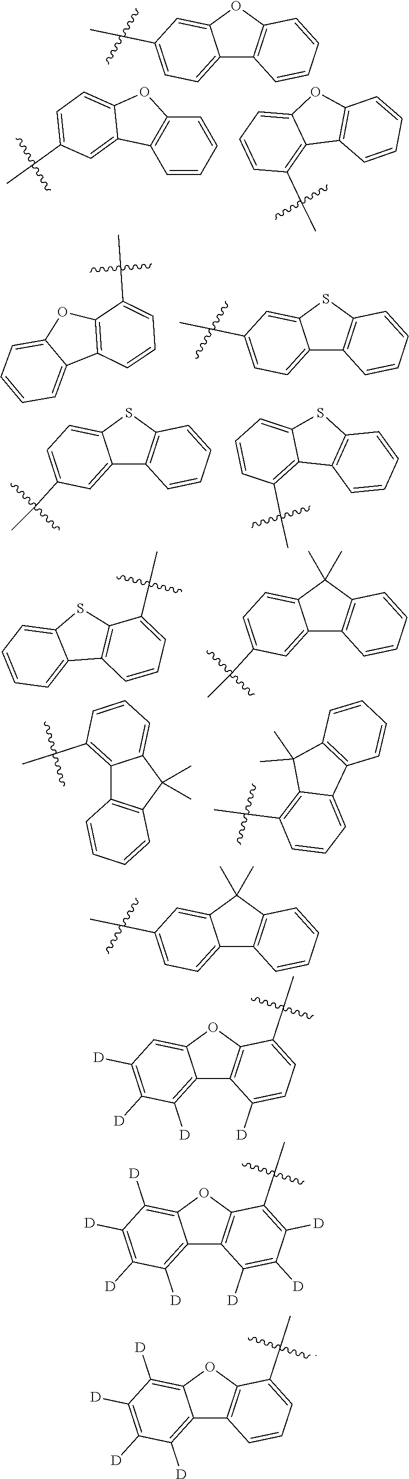

9. The arylamine compound according to claim 1, wherein 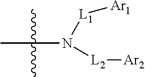
is selected from the following groups:

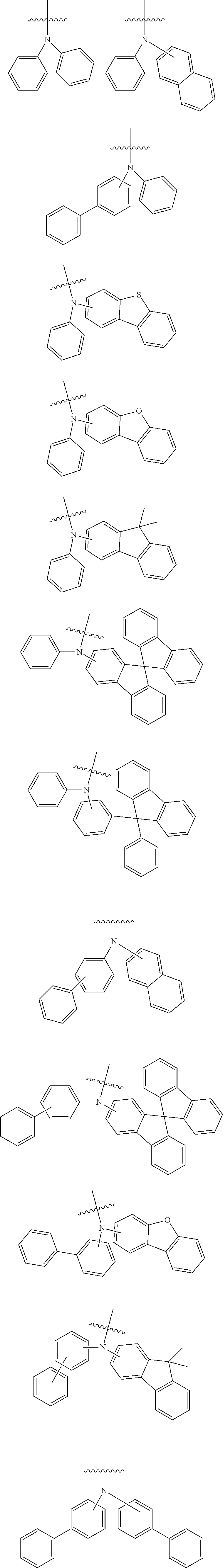

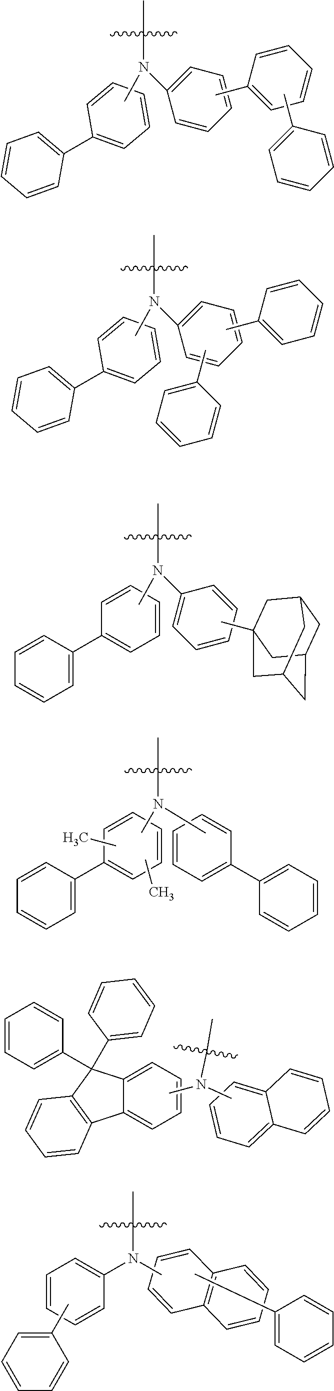

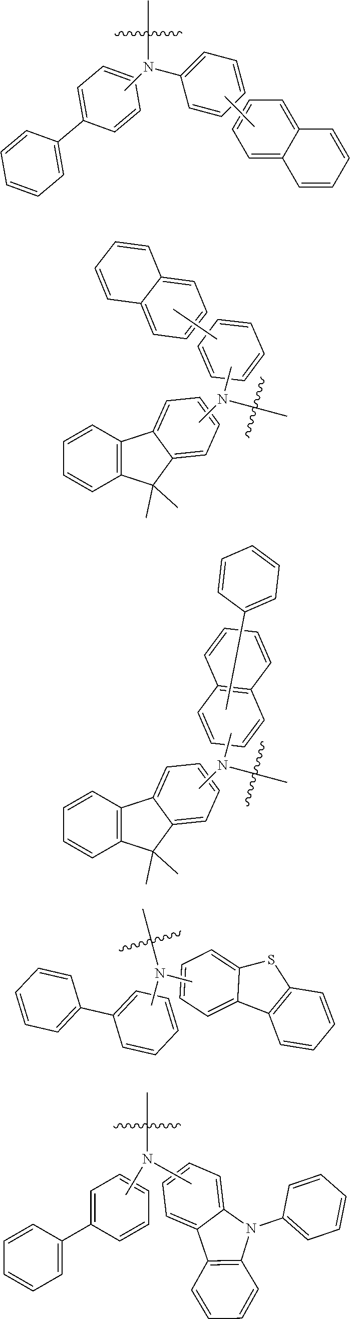

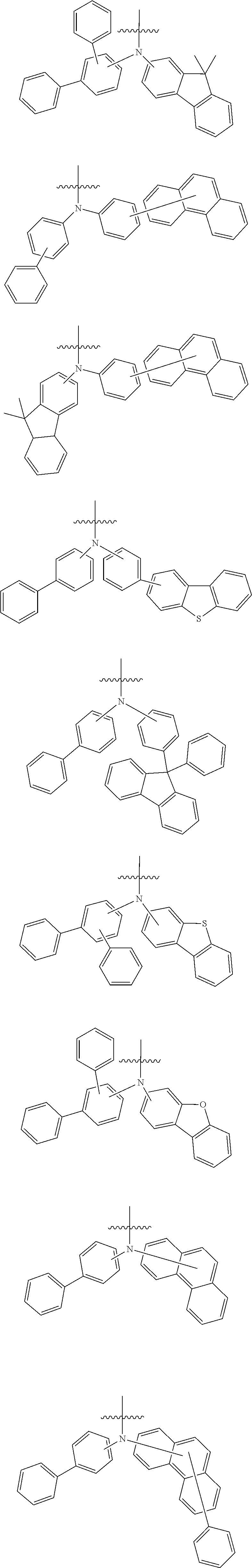

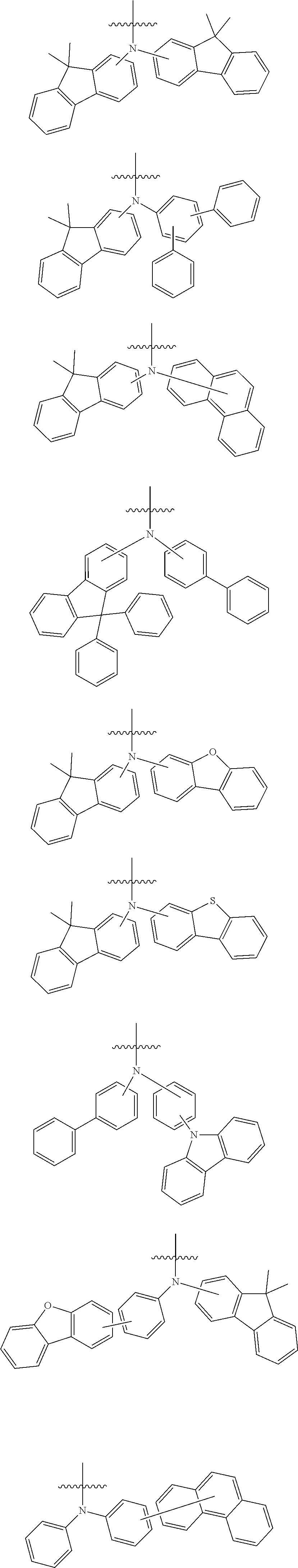

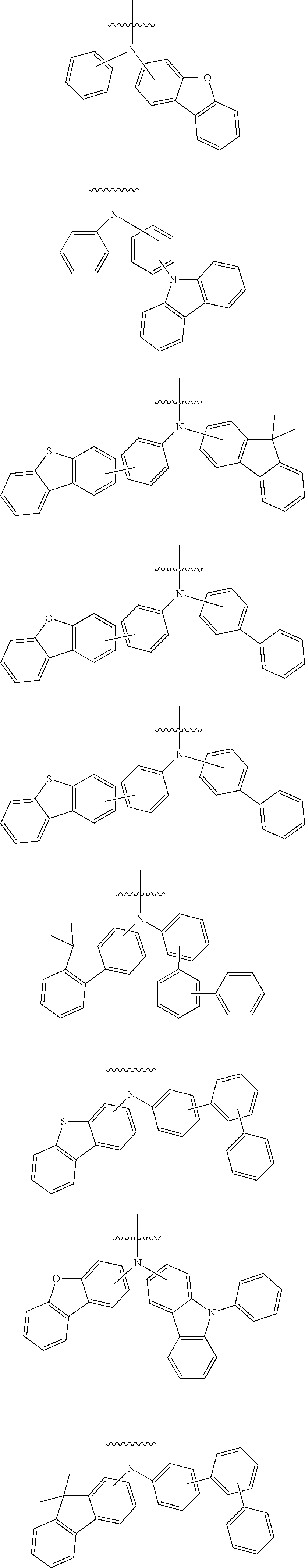

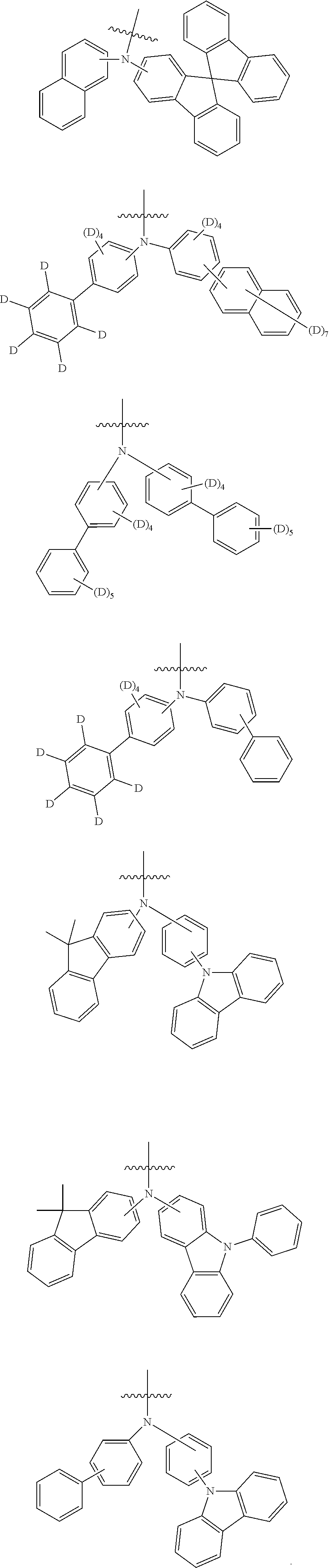

10. The arylamine compound according to claim 1, wherein the arylamine compound is selected from the group consisting of the following compounds:

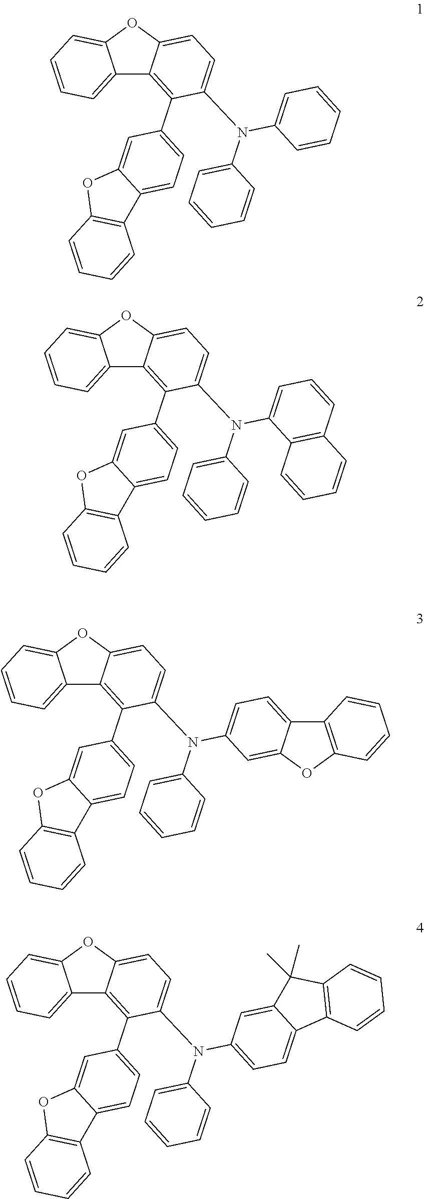

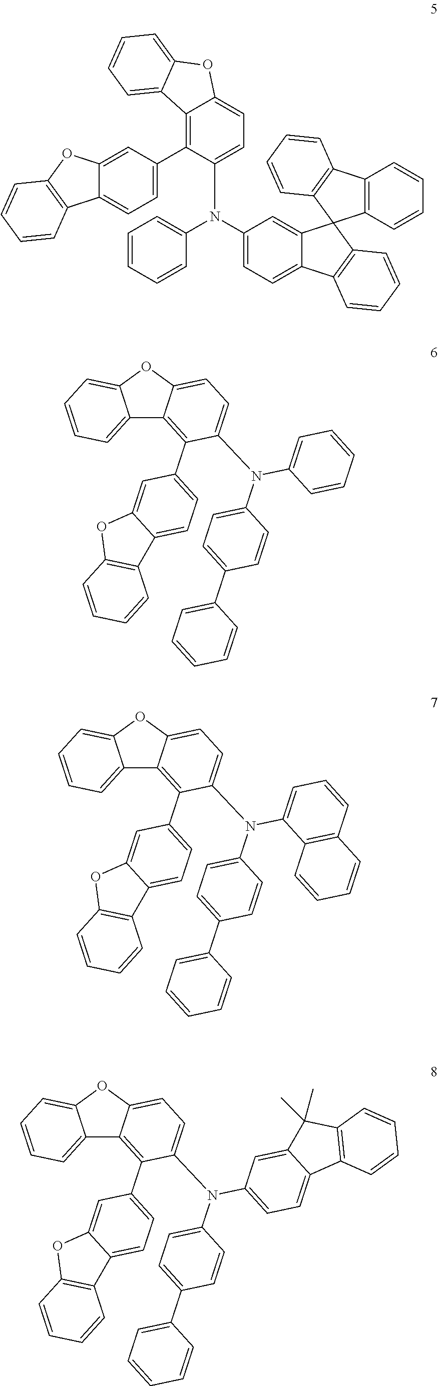 [SMILES: c1ccc(N(c2ccc3c(c2)C2(c4ccccc4-c4ccccc42)c2ccccc2-3)c2ccc3oc4ccccc4c3c2-c2ccc3c(c2)oc2ccccc23)cc1 ; c1ccc(-c2ccc(N(c3ccccc3)c3ccc4oc5ccccc5c4c3-c3ccc4c(c3)oc3ccccc34)cc2)cc1 ; c1ccc(-c2ccc(N(c3ccc4oc5ccccc5c4c3-c3ccc4c(c3)oc3ccccc34)c3cccc4ccccc34)cc2)cc1 ; CC1(C)c2ccccc2-c2ccc(N(c3ccc(-c4ccccc4)cc3)c3ccc4oc5ccccc5c4c3-c3ccc4c(c3)oc3ccccc34)cc21]

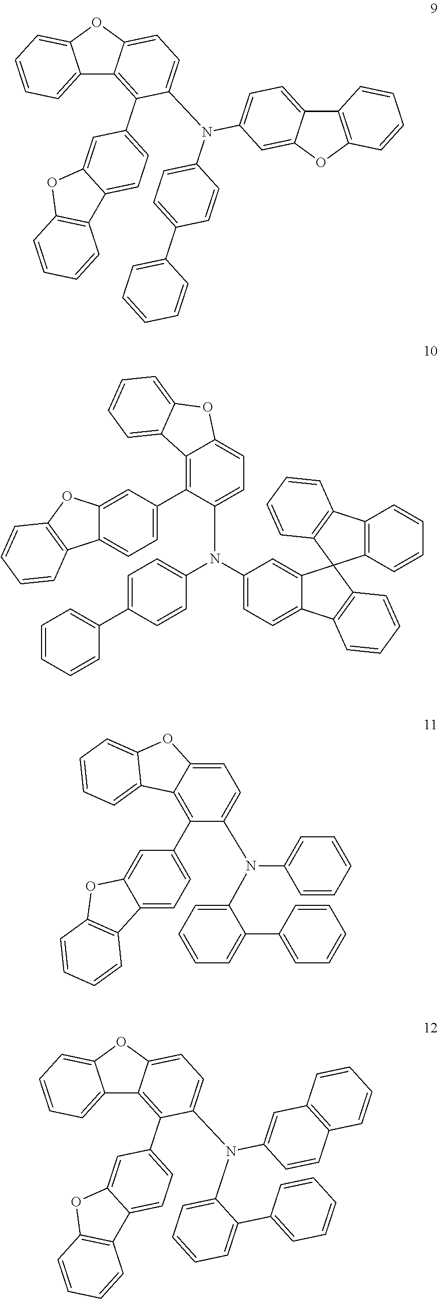 [SMILES: c1ccc(-c2ccc(N(c3ccc4c(c3)oc3ccccc34)c3ccc4oc5ccccc5c4c3-c3ccc4c(c3)oc3ccccc34)cc2)cc1 ; c1ccc(-c2ccc(N(c3ccc4c(c3)C3(c5ccccc5-c5ccccc53)c3ccccc3-4)c3ccc4oc5ccccc5c4c3-c3ccc4c(c3)oc3ccccc34)cc2)cc1 ; c1ccc(-c2ccccc2N(c2ccccc2)c2ccc3oc4ccccc4c3c2-c2ccc3c(c2)oc2ccccc23)cc1 ; c1ccc(-c2ccccc2N(c2ccc3ccccc3c2)c2ccc3oc4ccccc4c3c2-c2ccc3c(c2)oc2ccccc23)cc1]

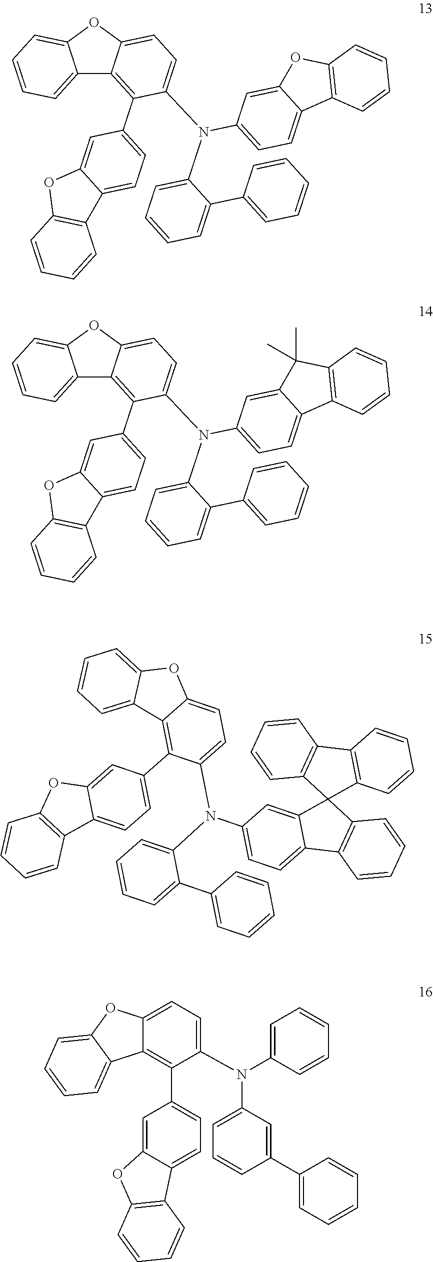 [SMILES: c1ccc(-c2ccccc2N(c2ccc3c(c2)oc2ccccc23)c2ccc3oc4ccccc4c3c2-c2ccc3c(c2)oc2ccccc23)cc1 ; CC1(C)c2ccccc2-c2ccc(N(c3ccccc3-c3ccccc3)c3ccc4oc5ccccc5c4c3-c3ccc4c(c3)oc3ccccc34)cc21 ; c1ccc(-c2ccccc2N(c2ccc3c(c2)C2(c4ccccc4-c4ccccc42)c2ccccc2-3)c2ccc3oc4ccccc4c3c2-c2ccc3c(c2)oc2ccccc23)cc1 ; c1ccc(-c2cccc(N(c3ccccc3)c3ccc4oc5ccccc5c4c3-c3ccc4c(c3)oc3ccccc34)c2)cc1]

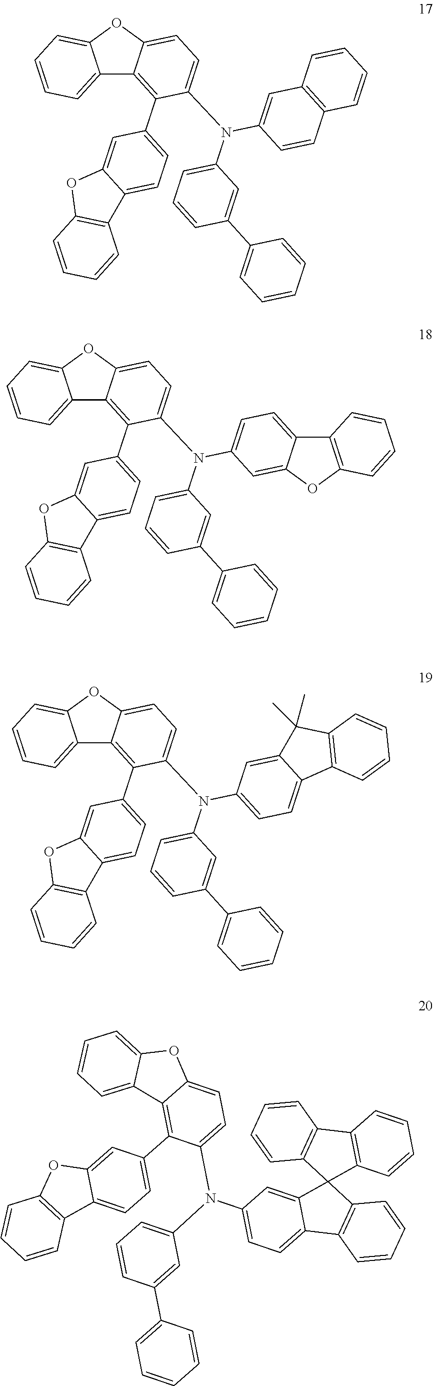 [SMILES: c1ccc(-c2cccc(N(c3ccc4ccccc4c3)c3ccc4oc5ccccc5c4c3-c3ccc4c(c3)oc3ccccc34)c2)cc1 ; c1ccc(-c2cccc(N(c3ccc4c(c3)oc3ccccc34)c3ccc4oc5ccccc5c4c3-c3ccc4c(c3)oc3ccccc34)c2)cc1 ; CC1(C)c2ccccc2-c2ccc(N(c3cccc(-c4ccccc4)c3)c3ccc4oc5ccccc5c4c3-c3ccc4c(c3)oc3ccccc34)cc21 ; c1ccc(-c2cccc(N(c3ccc4c(c3)C3(c5ccccc5-c5ccccc53)c3ccccc3-4)c3ccc4oc5ccccc5c4c3-c3ccc4c(c3)oc3ccccc34)c2)cc1]

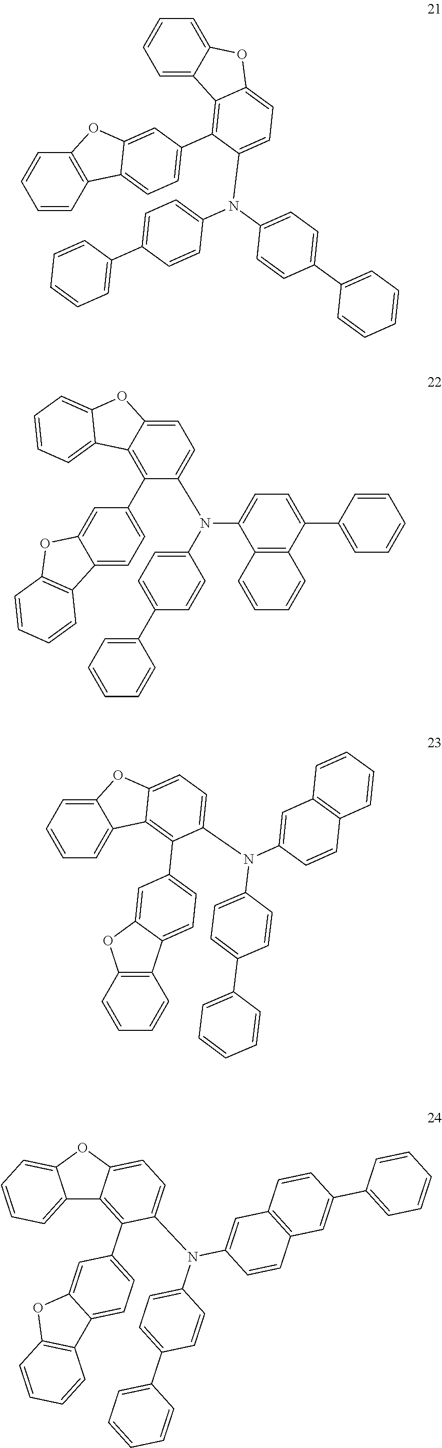 [SMILES: c1ccc(-c2ccc(N(c3ccc(-c4ccccc4)cc3)c3ccc4oc5ccccc5c4c3-c3ccc4c(c3)oc3ccccc34)cc2)cc1 ; c1ccc(-c2ccc(N(c3ccc4oc5ccccc5c4c3-c3ccc4c(c3)oc3ccccc34)c3ccc(-c4ccccc4)c4ccccc34)cc2)cc1 ; c1ccc(-c2ccc(N(c3ccc4ccccc4c3)c3ccc4oc5ccccc5c4c3-c3ccc4c(c3)oc3ccccc34)cc2)cc1 ; c1ccc(-c2ccc(N(c3ccc4cc(-c5ccccc5)ccc4c3)c3ccc4oc5ccccc5c4c3-c3ccc4c(c3)oc3ccccc34)cc2)cc1]

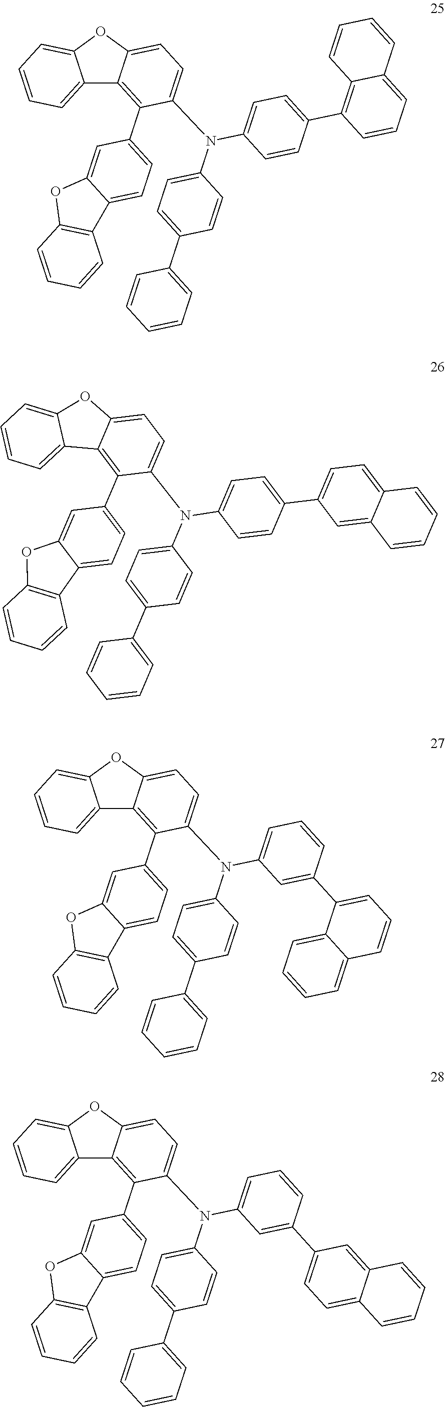 [SMILES: c1ccc(-c2ccc(N(c3ccc(-c4cccc5ccccc45)cc3)c3ccc4oc5ccccc5c4c3-c3ccc4c(c3)oc3ccccc34)cc2)cc1 ; c1ccc(-c2ccc(N(c3ccc(-c4ccc5ccccc5c4)cc3)c3ccc4oc5ccccc5c4c3-c3ccc4c(c3)oc3ccccc34)cc2)cc1 ; c1ccc(-c2ccc(N(c3cccc(-c4cccc5ccccc45)c3)c3ccc4oc5ccccc5c4c3-c3ccc4c(c3)oc3ccccc34)cc2)cc1 ; c1ccc(-c2ccc(N(c3cccc(-c4ccc5ccccc5c4)c3)c3ccc4oc5ccccc5c4c3-c3ccc4c(c3)oc3ccccc34)cc2)cc1]

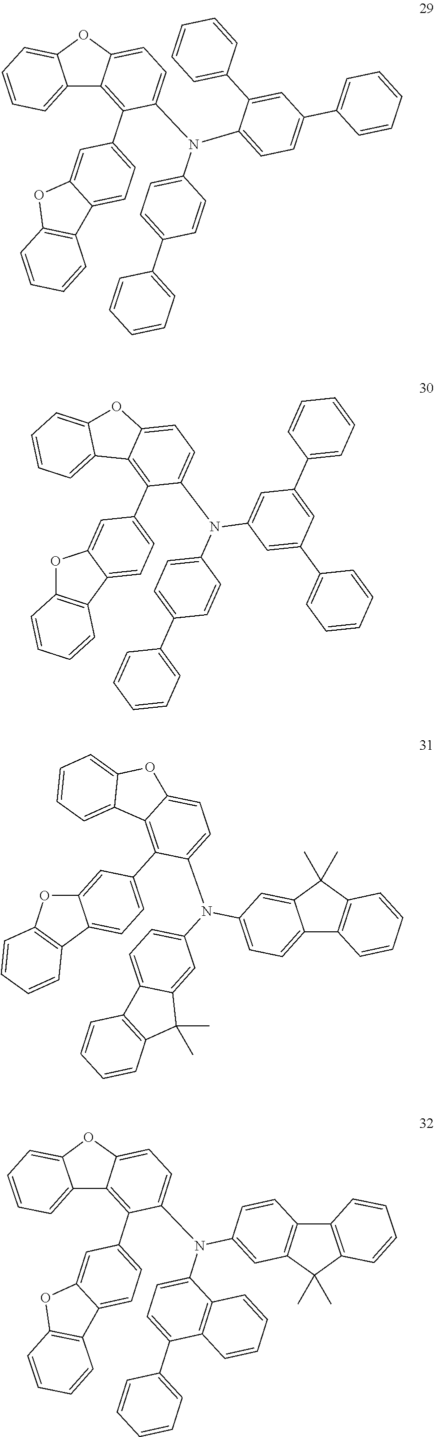 [SMILES: c1ccc(-c2ccc(N(c3ccc(-c4ccccc4)cc3-c3ccccc3)c3ccc4oc5ccccc5c4c3-c3ccc4c(c3)oc3ccccc34)cc2)cc1 ; c1ccc(-c2ccc(N(c3cc(-c4ccccc4)cc(-c4ccccc4)c3)c3ccc4oc5ccccc5c4c3-c3ccc4c(c3)oc3ccccc34)cc2)cc1 ; CC1(C)c2ccccc2-c2ccc(N(c3ccc4c(c3)C(C)(C)c3ccccc3-4)c3ccc4oc5ccccc5c4c3-c3ccc4c(c3)oc3ccccc34)cc21 ; CC1(C)c2ccccc2-c2ccc(N(c3ccc4oc5ccccc5c4c3-c3ccc4c(c3)oc3ccccc34)c3ccc(-c4ccccc4)c4ccccc34)cc21]

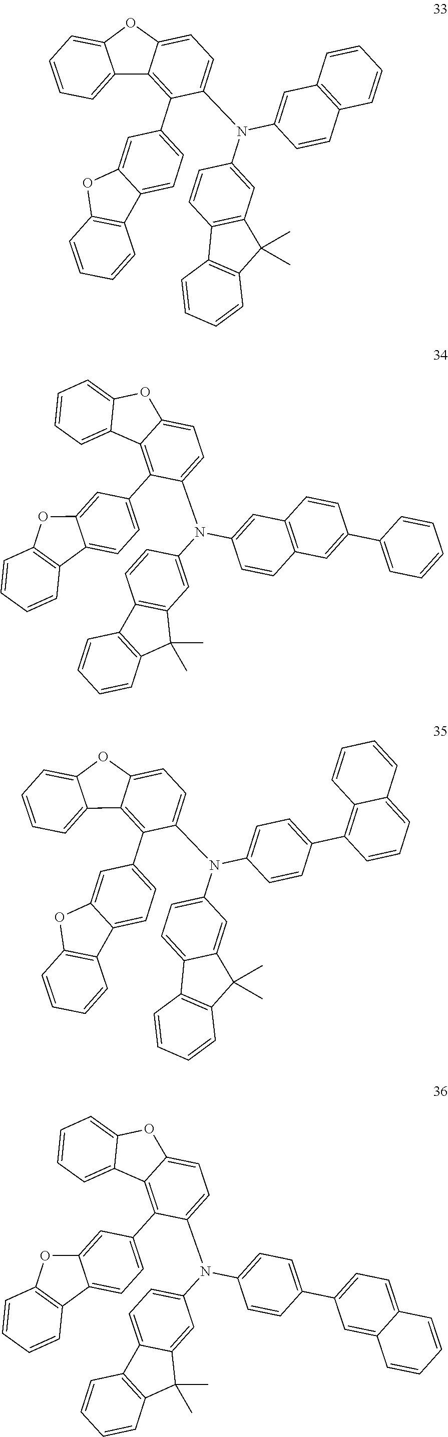 [SMILES: CC1(C)c2ccccc2-c2ccc(N(c3ccc4ccccc4c3)c3ccc4oc5ccccc5c4c3-c3ccc4c(c3)oc3ccccc34)cc21 ; CC1(C)c2ccccc2-c2ccc(N(c3ccc4cc(-c5ccccc5)ccc4c3)c3ccc4oc5ccccc5c4c3-c3ccc4c(c3)oc3ccccc34)cc21 ; CC1(C)c2ccccc2-c2ccc(N(c3ccc(-c4cccc5ccccc45)cc3)c3ccc4oc5ccccc5c4c3-c3ccc4c(c3)oc3ccccc34)cc21 ; CC1(C)c2ccccc2-c2ccc(N(c3ccc(-c4ccc5ccccc5c4)cc3)c3ccc4oc5ccccc5c4c3-c3ccc4c(c3)oc3ccccc34)cc21]

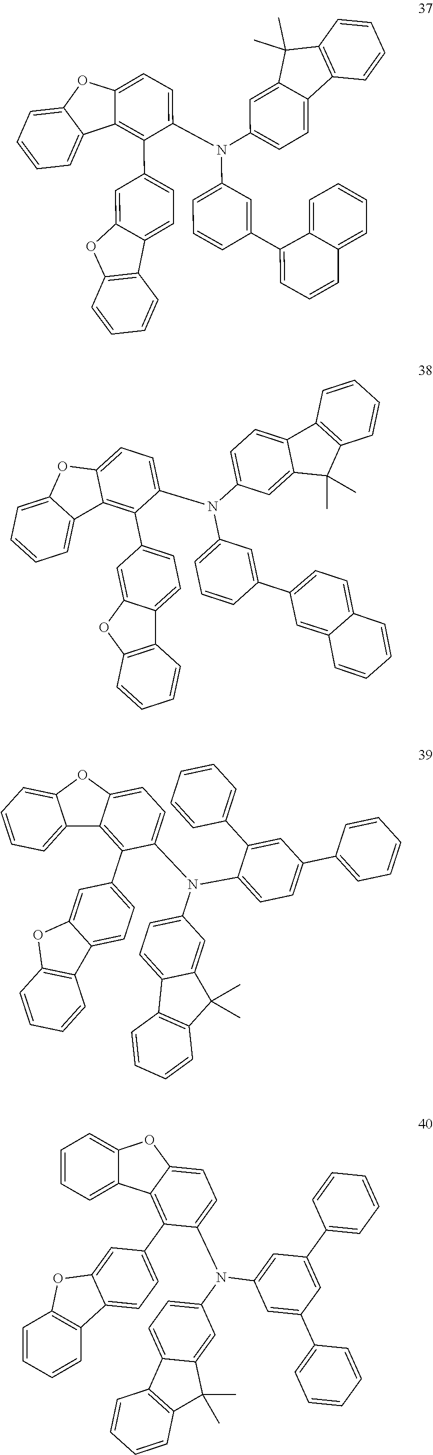 [SMILES: CC1(C)c2ccccc2-c2ccc(N(c3cccc(-c4cccc5ccccc45)c3)c3ccc4oc5ccccc5c4c3-c3ccc4c(c3)oc3ccccc34)cc21 ; CC1(C)c2ccccc2-c2ccc(N(c3cccc(-c4ccc5ccccc5c4)c3)c3ccc4oc5ccccc5c4c3-c3ccc4c(c3)oc3ccccc34)cc21 ; CC1(C)c2ccccc2-c2ccc(N(c3ccc(-c4ccccc4)cc3-c3ccccc3)c3ccc4oc5ccccc5c4c3-c3ccc4c(c3)oc3ccccc34)cc21 ; CC1(C)c2ccccc2-c2ccc(N(c3cc(-c4ccccc4)cc(-c4ccccc4)c3)c3ccc4oc5ccccc5c4c3-c3ccc4c(c3)oc3ccccc34)cc21]

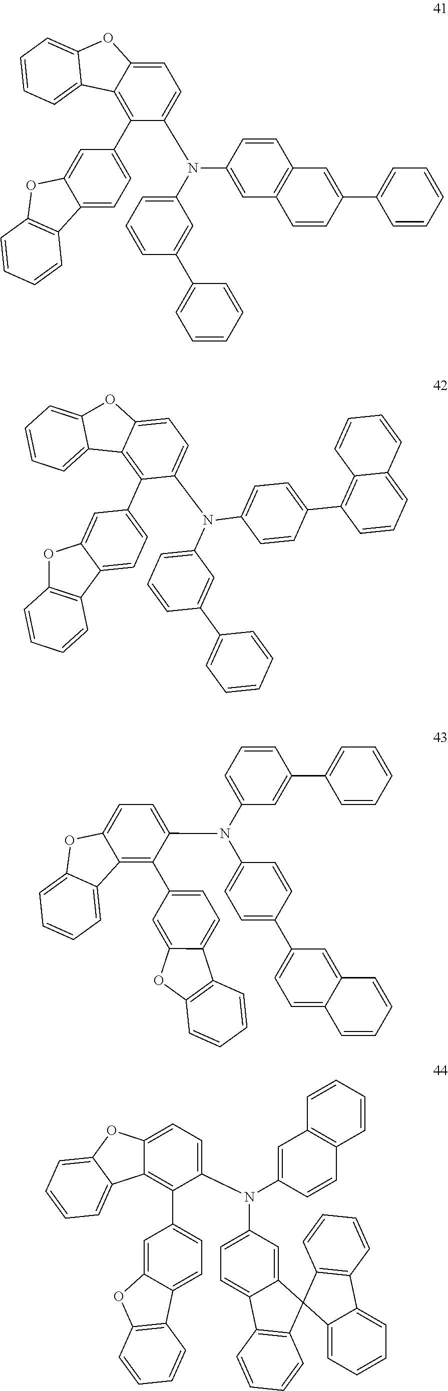

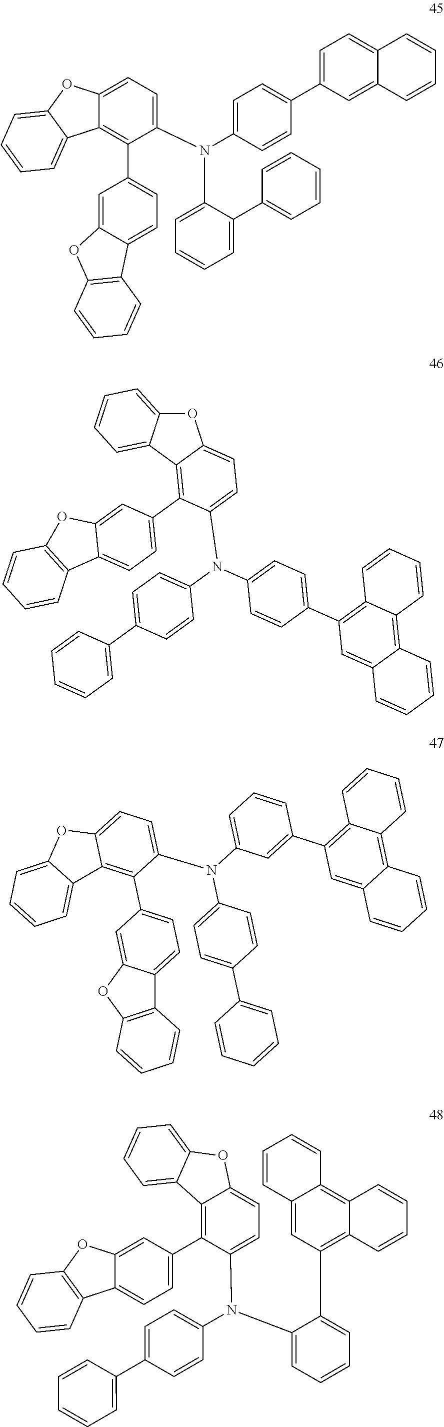 [SMILES: c1ccc(-c2ccccc2N(c2ccc(-c3ccc4ccccc4c3)cc2)c2ccc3oc4ccccc4c3c2-c2ccc3c(c2)oc2ccccc23)cc1 ; c1ccc(-c2ccc(N(c3ccc(-c4cc5ccccc5c5ccccc45)cc3)c3ccc4oc5ccccc5c4c3-c3ccc4c(c3)oc3ccccc34)cc2)cc1 ; c1ccc(-c2ccc(N(c3cccc(-c4cc5ccccc5c5ccccc45)c3)c3ccc4oc5ccccc5c4c3-c3ccc4c(c3)oc3ccccc34)cc2)cc1 ; c1ccc(-c2ccc(N(c3ccccc3-c3cc4ccccc4c4ccccc34)c3ccc4oc5ccccc5c4c3-c3ccc4c(c3)oc3ccccc34)cc2)cc1]

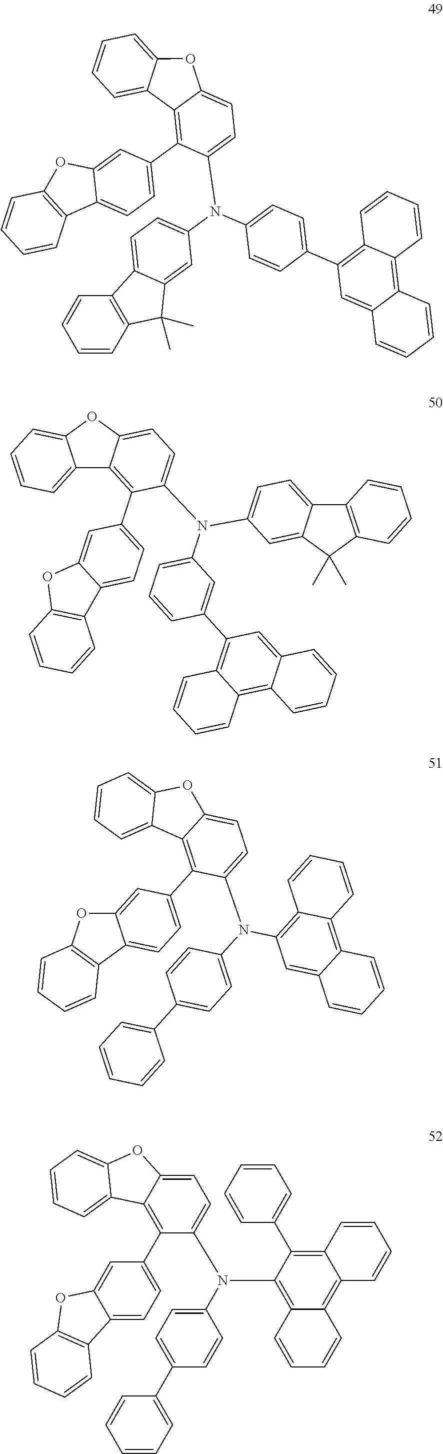 [SMILES: CC1(C)c2ccccc2-c2ccc(N(c3ccc(-c4cc5ccccc5c5ccccc45)cc3)c3ccc4oc5ccccc5c4c3-c3ccc4c(c3)oc3ccccc34)cc21 ; CC1(C)c2ccccc2-c2ccc(N(c3cccc(-c4cc5ccccc5c5ccccc45)c3)c3ccc4oc5ccccc5c4c3-c3ccc4c(c3)oc3ccccc34)cc21 ; c1ccc(-c2ccc(N(c3ccc4oc5ccccc5c4c3-c3ccc4c(c3)oc3ccccc34)c3cc4ccccc4c4ccccc34)cc2)cc1 ; c1ccc(-c2ccc(N(c3ccc4oc5ccccc5c4c3-c3ccc4c(c3)oc3ccccc34)c3c(-c4ccccc4)c4ccccc4c4ccccc34)cc2)cc1]

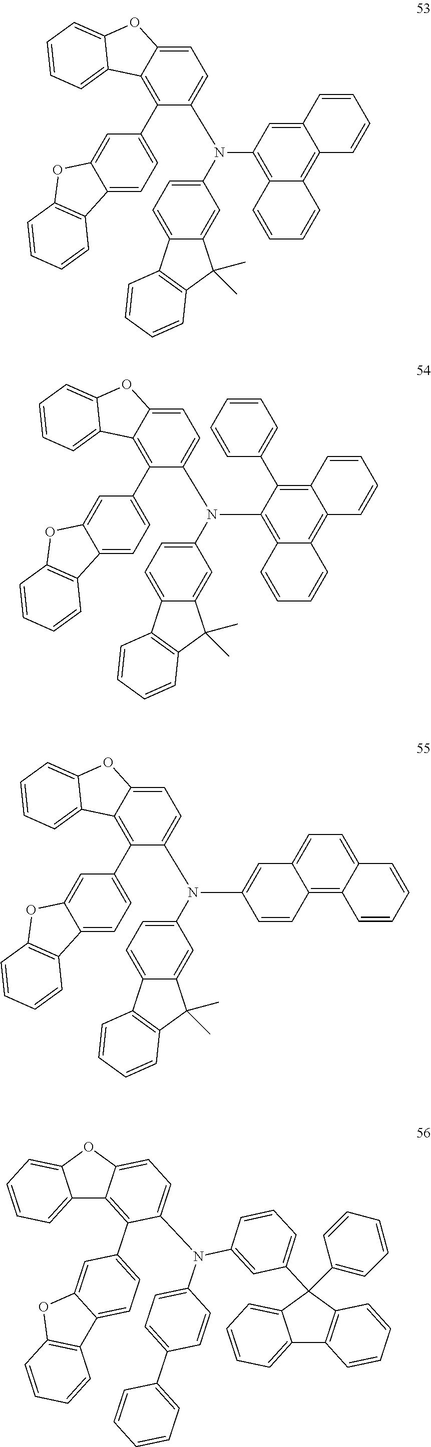 [SMILES: CC1(C)c2ccccc2-c2ccc(N(c3ccc4oc5ccccc5c4c3-c3ccc4c(c3)oc3ccccc34)c3cc4ccccc4c4ccccc34)cc21 ; CC1(C)c2ccccc2-c2ccc(N(c3ccc4oc5ccccc5c4c3-c3ccc4c(c3)oc3ccccc34)c3c(-c4ccccc4)c4ccccc4c4ccccc34)cc21 ; CC1(C)c2ccccc2-c2ccc(N(c3ccc4c(ccc5ccccc54)c3)c3ccc4oc5ccccc5c4c3-c3ccc4c(c3)oc3ccccc34)cc21 ; c1ccc(-c2ccc(N(c3cccc(C4(c5ccccc5)c5ccccc5-c5ccccc54)c3)c3ccc4oc5ccccc5c4c3-c3ccc4c(c3)oc3ccccc34)cc2)cc1]

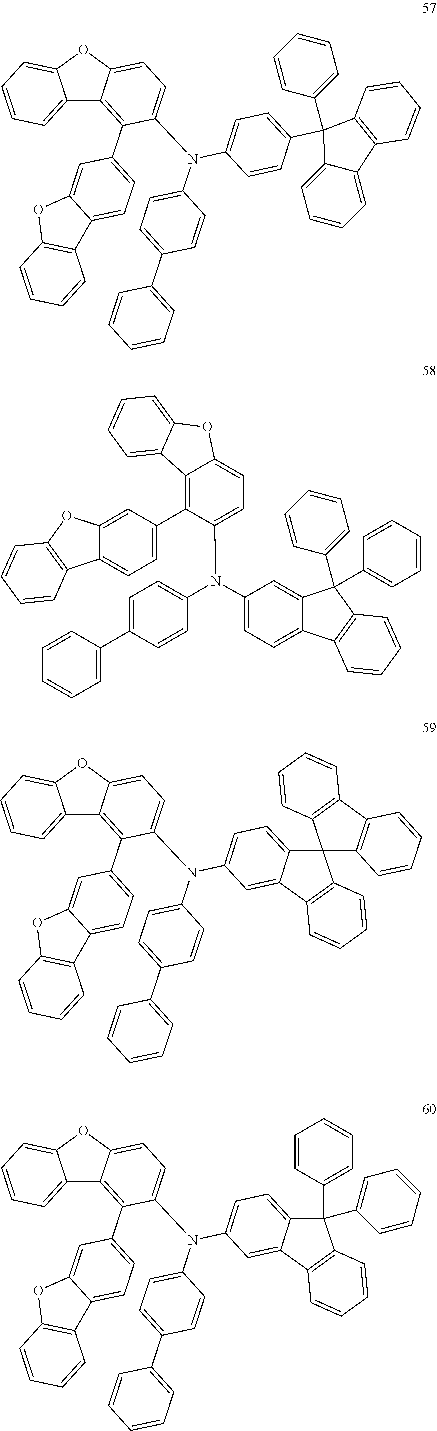 [SMILES: c1ccc(-c2ccc(N(c3ccc(C4(c5ccccc5)c5ccccc5-c5ccccc54)cc3)c3ccc4oc5ccccc5c4c3-c3ccc4c(c3)oc3ccccc34)cc2)cc1 ; c1ccc(-c2ccc(N(c3ccc4c(c3)C(c3ccccc3)(c3ccccc3)c3ccccc3-4)c3ccc4oc5ccccc5c4c3-c3ccc4c(c3)oc3ccccc34)cc2)cc1 ; c1ccc(-c2ccc(N(c3ccc4c(c3)-c3ccccc3C43c4ccccc4-c4ccccc43)c3ccc4oc5ccccc5c4c3-c3ccc4c(c3)oc3ccccc34)cc2)cc1 ; c1ccc(-c2ccc(N(c3ccc4c(c3)-c3ccccc3C4(c3ccccc3)c3ccccc3)c3ccc4oc5ccccc5c4c3-c3ccc4c(c3)oc3ccccc34)cc2)cc1]

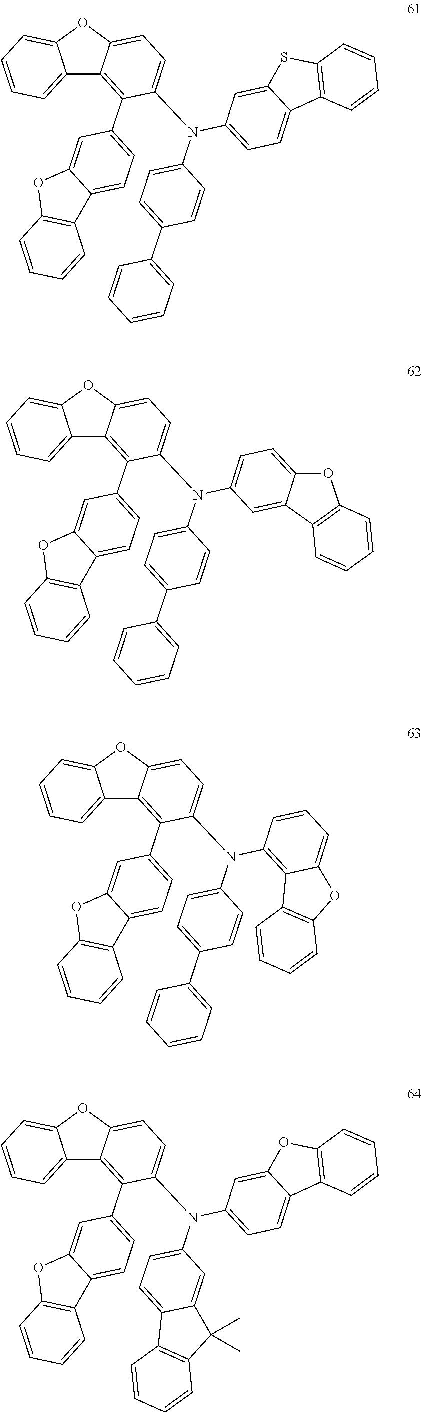 [SMILES: c1ccc(-c2ccc(N(c3ccc4c(c3)sc3ccccc34)c3ccc4oc5ccccc5c4c3-c3ccc4c(c3)oc3ccccc34)cc2)cc1 ; c1ccc(-c2ccc(N(c3ccc4oc5ccccc5c4c3)c3ccc4oc5ccccc5c4c3-c3ccc4c(c3)oc3ccccc34)cc2)cc1 ; c1ccc(-c2ccc(N(c3ccc4oc5ccccc5c4c3-c3ccc4c(c3)oc3ccccc34)c3cccc4oc5ccccc5c34)cc2)cc1 ; CC1(C)c2ccccc2-c2ccc(N(c3ccc4c(c3)oc3ccccc34)c3ccc4oc5ccccc5c4c3-c3ccc4c(c3)oc3ccccc34)cc21]

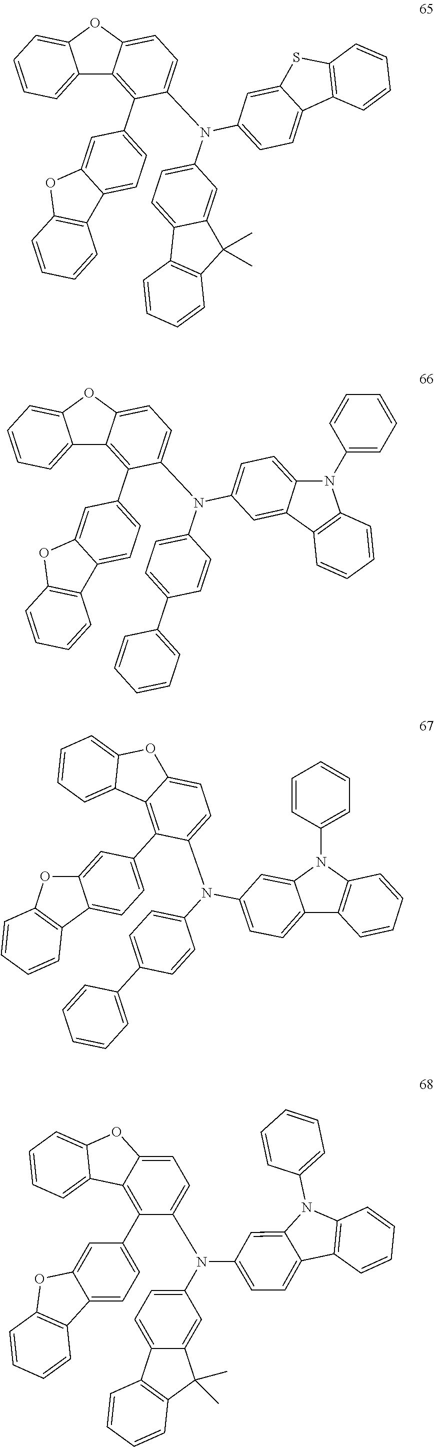 [SMILES: CC1(C)c2ccccc2-c2ccc(N(c3ccc4c(c3)sc3ccccc34)c3ccc4oc5ccccc5c4c3-c3ccc4c(c3)oc3ccccc34)cc21 ; c1ccc(-c2ccc(N(c3ccc4c(c3)c3ccccc3n4-c3ccccc3)c3ccc4oc5ccccc5c4c3-c3ccc4c(c3)oc3ccccc34)cc2)cc1 ; c1ccc(-c2ccc(N(c3ccc4c5ccccc5n(-c5ccccc5)c4c3)c3ccc4oc5ccccc5c4c3-c3ccc4c(c3)oc3ccccc34)cc2)cc1 ; CC1(C)c2ccccc2-c2ccc(N(c3ccc4c5ccccc5n(-c5ccccc5)c4c3)c3ccc4oc5ccccc5c4c3-c3ccc4c(c3)oc3ccccc34)cc21]

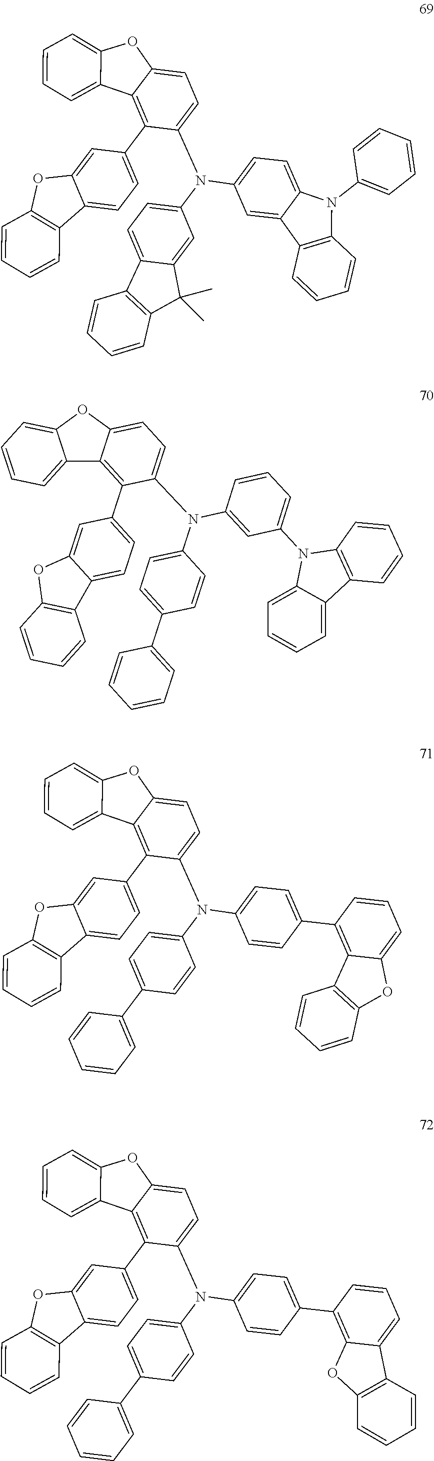 [SMILES: CC1(C)c2ccccc2-c2ccc(N(c3ccc4c(c3)c3ccccc3n4-c3ccccc3)c3ccc4oc5ccccc5c4c3-c3ccc4c(c3)oc3ccccc34)cc21 ; c1ccc(-c2ccc(N(c3cccc(-n4c5ccccc5c5ccccc54)c3)c3ccc4oc5ccccc5c4c3-c3ccc4c(c3)oc3ccccc34)cc2)cc1 ; c1ccc(-c2ccc(N(c3ccc(-c4cccc5oc6ccccc6c45)cc3)c3ccc4oc5ccccc5c4c3-c3ccc4c(c3)oc3ccccc34)cc2)cc1 ; c1ccc(-c2ccc(N(c3ccc(-c4cccc5c4oc4ccccc45)cc3)c3ccc4oc5ccccc5c4c3-c3ccc4c(c3)oc3ccccc34)cc2)cc1]

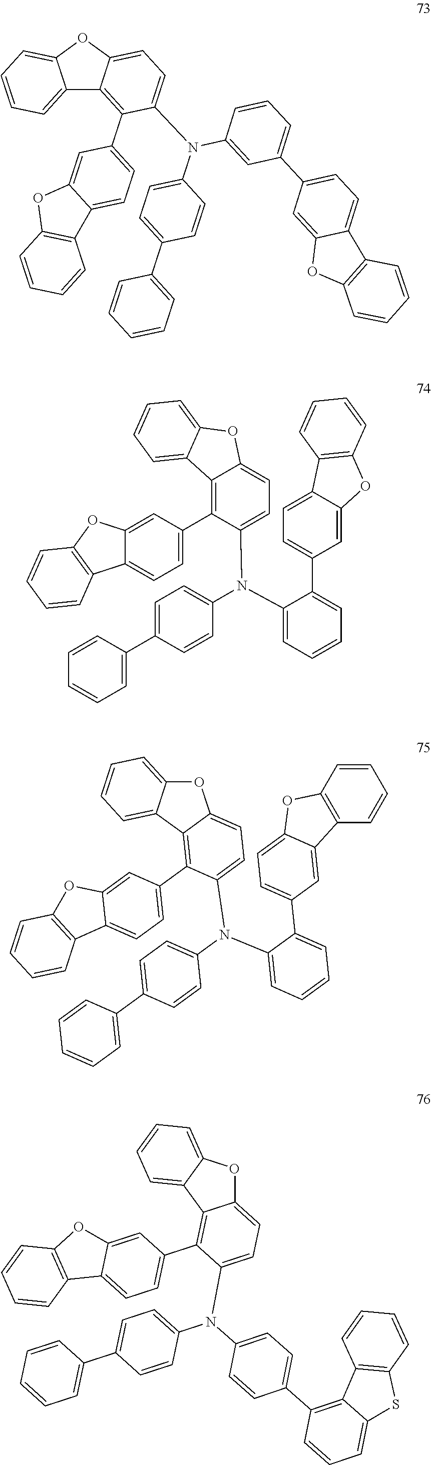 [SMILES: c1ccc(-c2ccc(N(c3cccc(-c4ccc5c(c4)oc4ccccc45)c3)c3ccc4oc5ccccc5c4c3-c3ccc4c(c3)oc3ccccc34)cc2)cc1 ; c1ccc(-c2ccc(N(c3ccccc3-c3ccc4c(c3)oc3ccccc34)c3ccc4oc5ccccc5c4c3-c3ccc4c(c3)oc3ccccc34)cc2)cc1 ; c1ccc(-c2ccc(N(c3ccccc3-c3ccc4oc5ccccc5c4c3)c3ccc4oc5ccccc5c4c3-c3ccc4c(c3)oc3ccccc34)cc2)cc1 ; c1ccc(-c2ccc(N(c3ccc(-c4cccc5sc6ccccc6c45)cc3)c3ccc4oc5ccccc5c4c3-c3ccc4c(c3)oc3ccccc34)cc2)cc1]

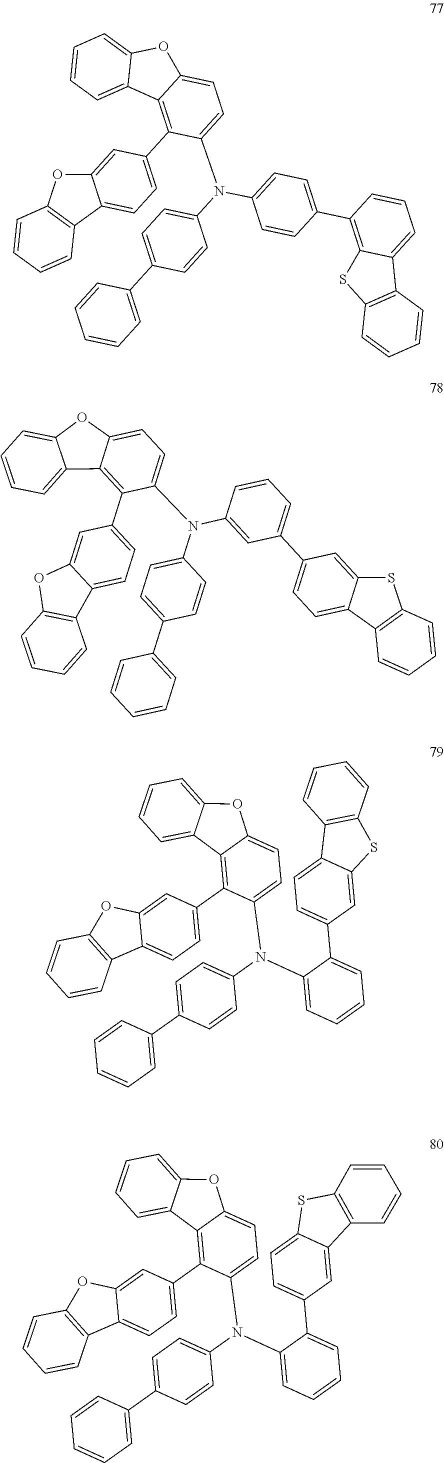 [SMILES: c1ccc(-c2ccc(N(c3ccc(-c4cccc5c4sc4ccccc45)cc3)c3ccc4oc5ccccc5c4c3-c3ccc4c(c3)oc3ccccc34)cc2)cc1 ; c1ccc(-c2ccc(N(c3cccc(-c4ccc5c(c4)sc4ccccc45)c3)c3ccc4oc5ccccc5c4c3-c3ccc4c(c3)oc3ccccc34)cc2)cc1 ; c1ccc(-c2ccc(N(c3ccccc3-c3ccc4c(c3)sc3ccccc34)c3ccc4oc5ccccc5c4c3-c3ccc4c(c3)oc3ccccc34)cc2)cc1 ; c1ccc(-c2ccc(N(c3ccccc3-c3ccc4sc5ccccc5c4c3)c3ccc4oc5ccccc5c4c3-c3ccc4c(c3)oc3ccccc34)cc2)cc1]

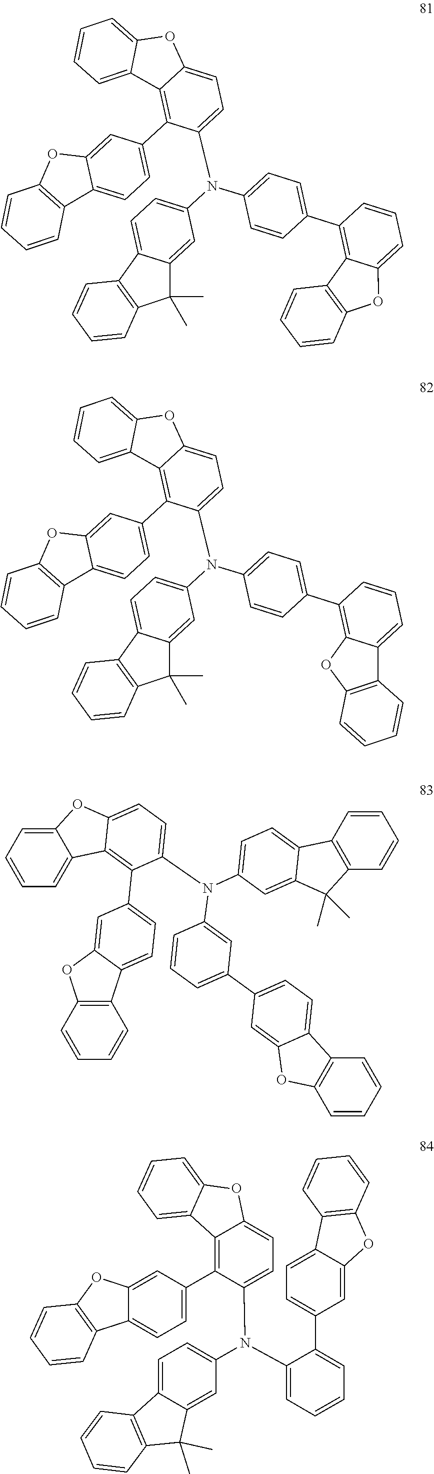

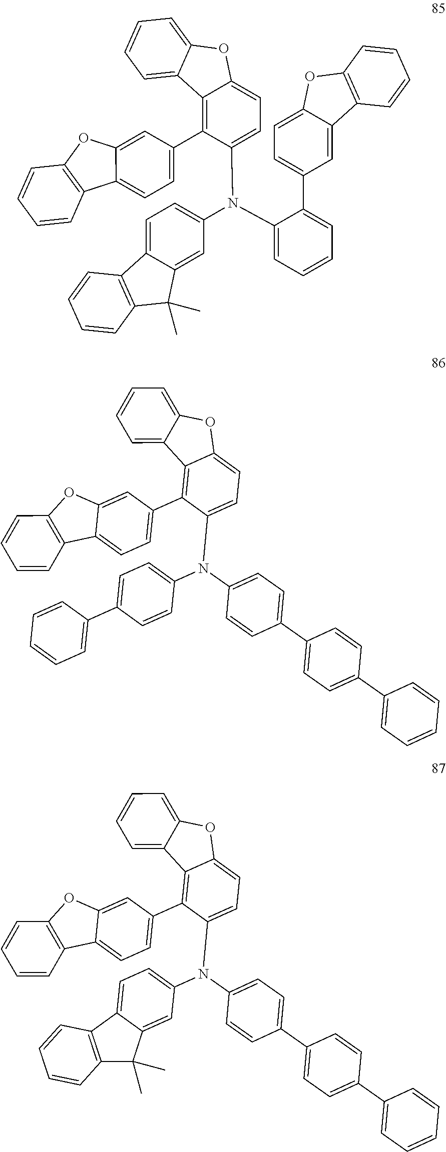 [SMILES: CC1(C)c2ccccc2-c2ccc(N(c3ccccc3-c3ccc4oc5ccccc5c4c3)c3ccc4oc5ccccc5c4c3-c3ccc4c(c3)oc3ccccc34)cc21 ; c1ccc(-c2ccc(-c3ccc(N(c4ccc(-c5ccccc5)cc4)c4ccc5oc6ccccc6c5c4-c4ccc5c(c4)oc4ccccc45)cc3)cc2)cc1 ; CC1(C)c2ccccc2-c2ccc(N(c3ccc(-c4ccc(-c5ccccc5)cc4)cc3)c3ccc4oc5ccccc5c4c3-c3ccc4c(c3)oc3ccccc34)cc21]

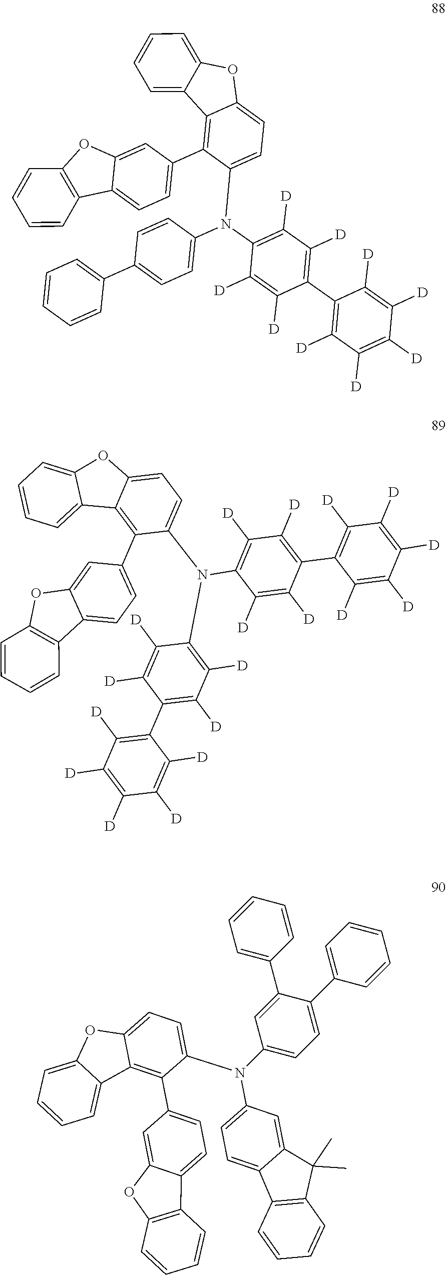 [SMILES: [2H]c1c([2H])c([2H])c(-c2c([2H])c([2H])c(N(c3ccc(-c4ccccc4)cc3)c3ccc4oc5ccccc5c4c3-c3ccc4c(c3)oc3ccccc34)c([2H])c2[2H])c([2H])c1[2H] ; [2H]c1c([2H])c([2H])c(-c2c([2H])c([2H])c(N(c3ccc4oc5ccccc5c4c3-c3ccc4c(c3)oc3ccccc34)c3c([2H])c([2H])c(-c4c([2H])c([2H])c([2H])c([2H])c4[2H])c([2H])c3[2H])c([2H])c2[2H])c([2H])c1[2H] ; CC1(C)c2ccccc2-c2ccc(N(c3ccc(-c4ccccc4)c(-c4ccccc4)c3)c3ccc4oc5ccccc5c4c3-c3ccc4c(c3)oc3ccccc34)cc21]

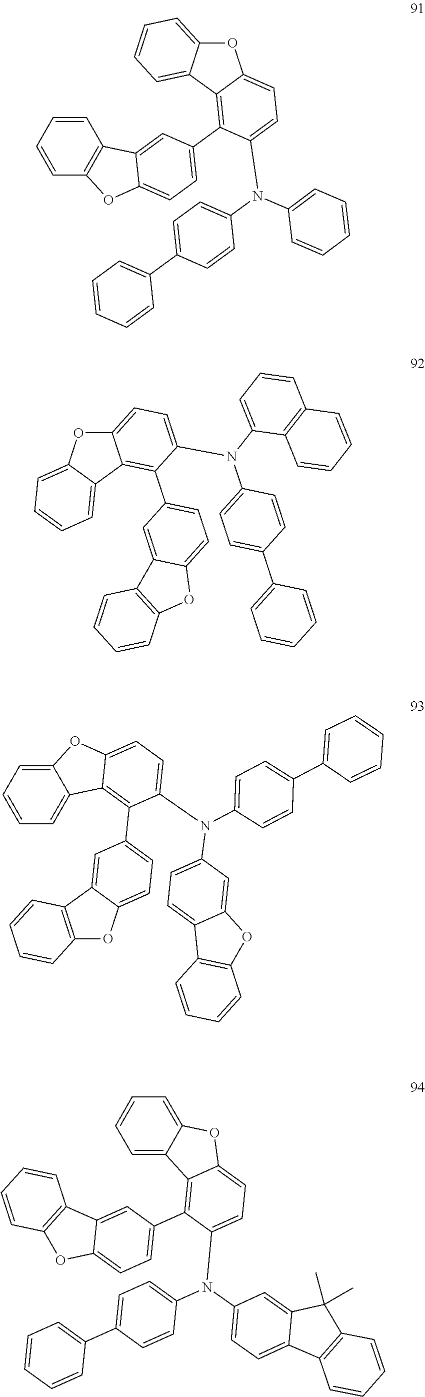 [SMILES: c1ccc(-c2ccc(N(c3ccccc3)c3ccc4oc5ccccc5c4c3-c3ccc4oc5ccccc5c4c3)cc2)cc1 ; c1ccc(-c2ccc(N(c3ccc4oc5ccccc5c4c3-c3ccc4oc5ccccc5c4c3)c3cccc4ccccc34)cc2)cc1 ; c1ccc(-c2ccc(N(c3ccc4c(c3)oc3ccccc34)c3ccc4oc5ccccc5c4c3-c3ccc4oc5ccccc5c4c3)cc2)cc1 ; CC1(C)c2ccccc2-c2ccc(N(c3ccc(-c4ccccc4)cc3)c3ccc4oc5ccccc5c4c3-c3ccc4oc5ccccc5c4c3)cc21]

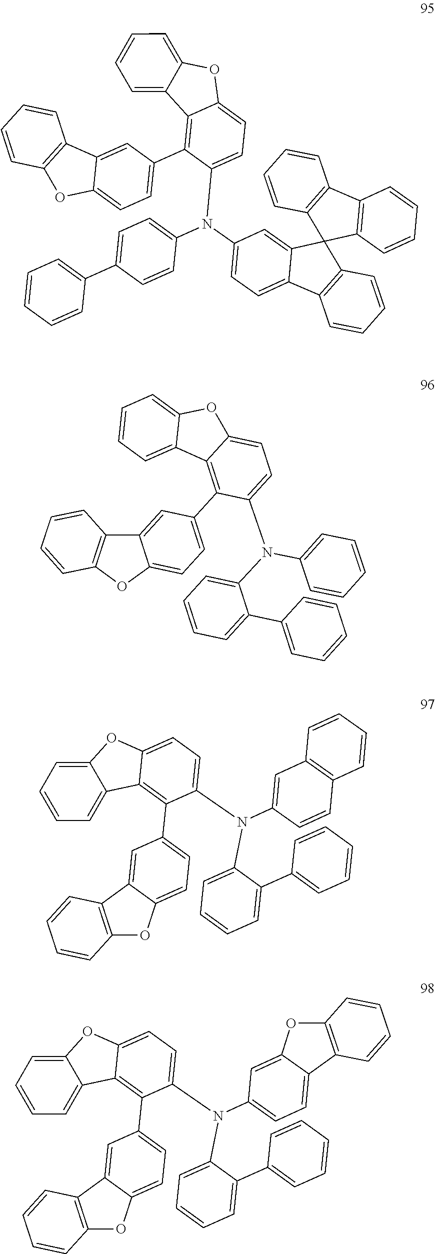 [SMILES: c1ccc(-c2ccc(N(c3ccc4c(c3)C3(c5ccccc5-c5ccccc53)c3ccccc3-4)c3ccc4oc5ccccc5c4c3-c3ccc4oc5ccccc5c4c3)cc2)cc1 ; c1ccc(-c2ccccc2N(c2ccccc2)c2ccc3oc4ccccc4c3c2-c2ccc3oc4ccccc4c3c2)cc1 ; c1ccc(-c2ccccc2N(c2ccc3ccccc3c2)c2ccc3oc4ccccc4c3c2-c2ccc3oc4ccccc4c3c2)cc1 ; c1ccc(-c2ccccc2N(c2ccc3c(c2)oc2ccccc23)c2ccc3oc4ccccc4c3c2-c2ccc3oc4ccccc4c3c2)cc1]

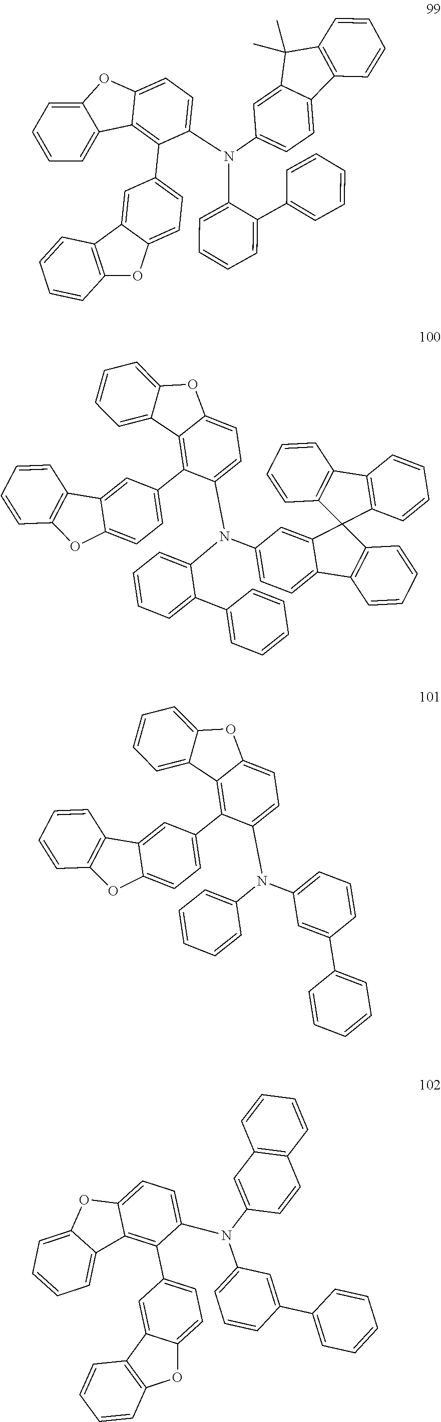 [SMILES: CC1(C)c2ccccc2-c2ccc(N(c3ccccc3-c3ccccc3)c3ccc4oc5ccccc5c4c3-c3ccc4oc5ccccc5c4c3)cc21 ; c1ccc(-c2ccccc2N(c2ccc3c(c2)C2(c4ccccc4-c4ccccc42)c2ccccc2-3)c2ccc3oc4ccccc4c3c2-c2ccc3oc4ccccc4c3c2)cc1 ; c1ccc(-c2cccc(N(c3ccccc3)c3ccc4oc5ccccc5c4c3-c3ccc4oc5ccccc5c4c3)c2)cc1 ; c1ccc(-c2cccc(N(c3ccc4ccccc4c3)c3ccc4oc5ccccc5c4c3-c3ccc4oc5ccccc5c4c3)c2)cc1]

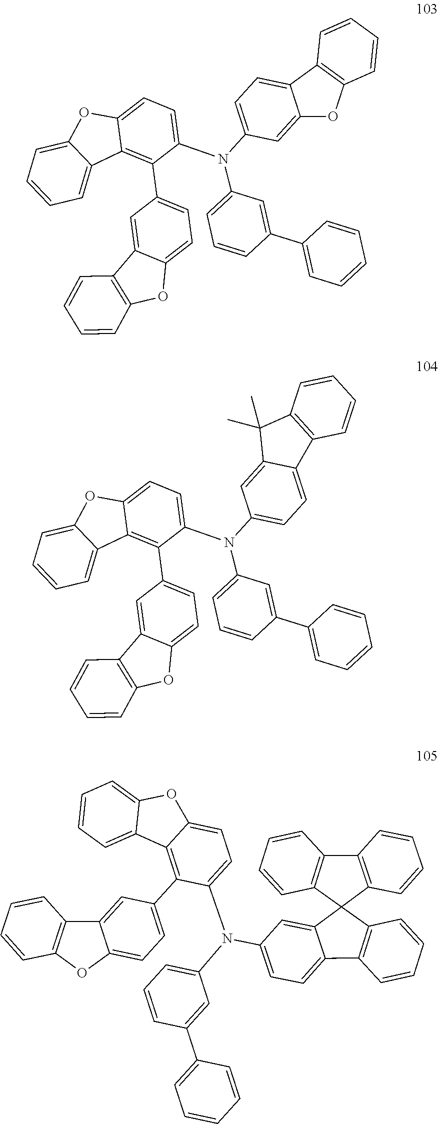 [SMILES: c1ccc(-c2cccc(N(c3ccc4c(c3)oc3ccccc34)c3ccc4oc5ccccc5c4c3-c3ccc4oc5ccccc5c4c3)c2)cc1 ; CC1(C)c2ccccc2-c2ccc(N(c3cccc(-c4ccccc4)c3)c3ccc4oc5ccccc5c4c3-c3ccc4oc5ccccc5c4c3)cc21 ; c1ccc(-c2cccc(N(c3ccc4c(c3)C3(c5ccccc5-c5ccccc53)c3ccccc3-4)c3ccc4oc5ccccc5c4c3-c3ccc4oc5ccccc5c4c3)c2)cc1]

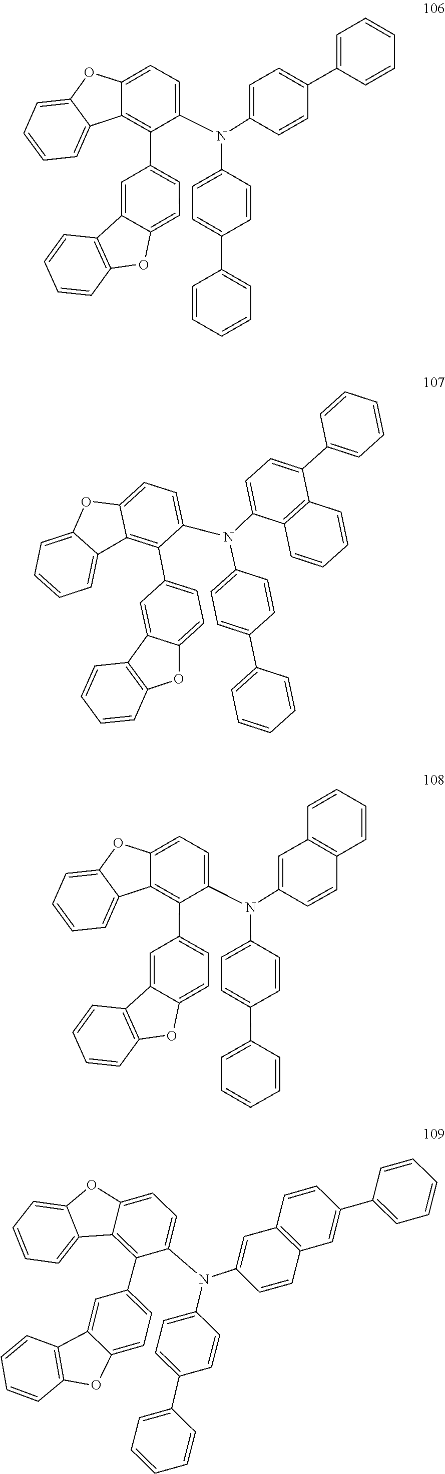 [SMILES: c1ccc(-c2ccc(N(c3ccc(-c4ccccc4)cc3)c3ccc4oc5ccccc5c4c3-c3ccc4oc5ccccc5c4c3)cc2)cc1 ; c1ccc(-c2ccc(N(c3ccc4oc5ccccc5c4c3-c3ccc4oc5ccccc5c4c3)c3ccc(-c4ccccc4)c4ccccc34)cc2)cc1 ; c1ccc(-c2ccc(N(c3ccc4ccccc4c3)c3ccc4oc5ccccc5c4c3-c3ccc4oc5ccccc5c4c3)cc2)cc1 ; c1ccc(-c2ccc(N(c3ccc4cc(-c5ccccc5)ccc4c3)c3ccc4oc5ccccc5c4c3-c3ccc4oc5ccccc5c4c3)cc2)cc1]

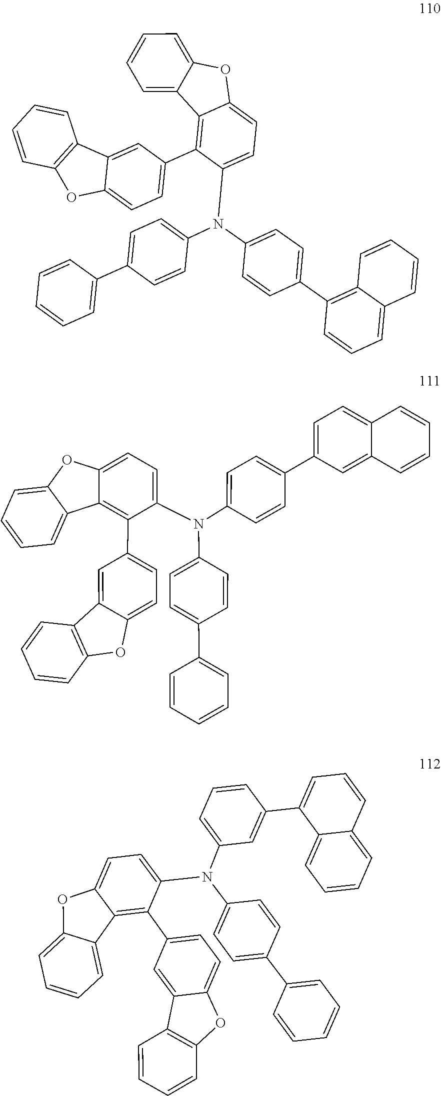 [SMILES: c1ccc(-c2ccc(N(c3ccc(-c4cccc5ccccc45)cc3)c3ccc4oc5ccccc5c4c3-c3ccc4oc5ccccc5c4c3)cc2)cc1 ; c1ccc(-c2ccc(N(c3ccc(-c4ccc5ccccc5c4)cc3)c3ccc4oc5ccccc5c4c3-c3ccc4oc5ccccc5c4c3)cc2)cc1 ; c1ccc(-c2ccc(N(c3cccc(-c4cccc5ccccc45)c3)c3ccc4oc5ccccc5c4c3-c3ccc4oc5ccccc5c4c3)cc2)cc1]

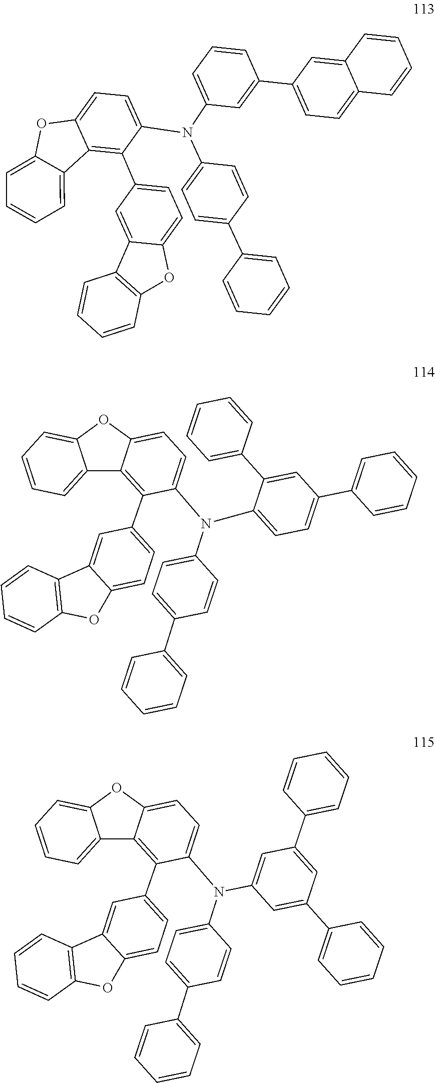 [SMILES: c1ccc(-c2ccc(N(c3cccc(-c4ccc5ccccc5c4)c3)c3ccc4oc5ccccc5c4c3-c3ccc4oc5ccccc5c4c3)cc2)cc1 ; c1ccc(-c2ccc(N(c3ccc(-c4ccccc4)cc3-c3ccccc3)c3ccc4oc5ccccc5c4c3-c3ccc4oc5ccccc5c4c3)cc2)cc1 ; c1ccc(-c2ccc(N(c3cc(-c4ccccc4)cc(-c4ccccc4)c3)c3ccc4oc5ccccc5c4c3-c3ccc4oc5ccccc5c4c3)cc2)cc1]

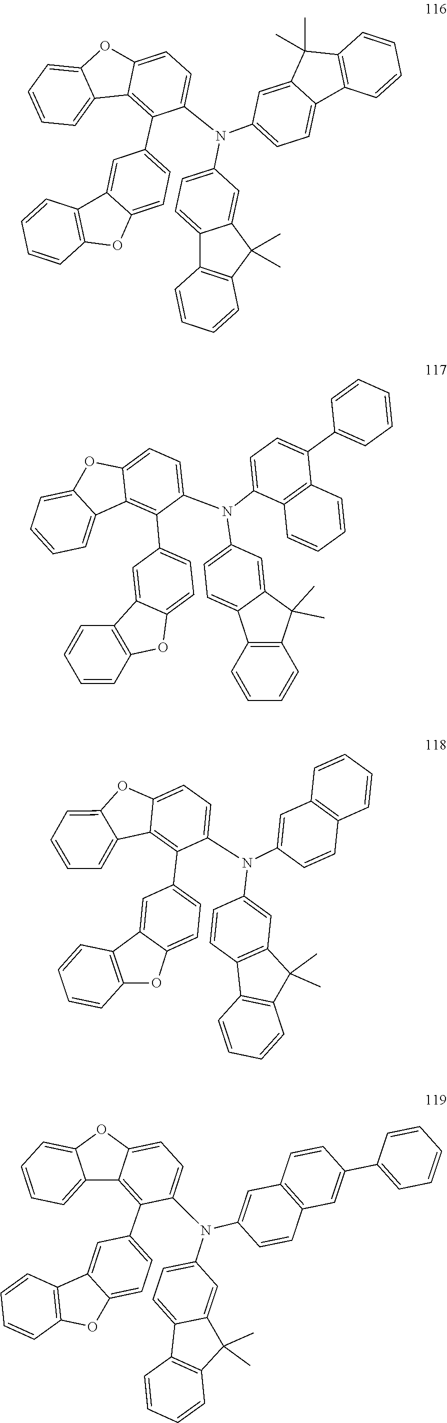 [SMILES: CC1(C)c2ccccc2-c2ccc(N(c3ccc4c(c3)C(C)(C)c3ccccc3-4)c3ccc4oc5ccccc5c4c3-c3ccc4oc5ccccc5c4c3)cc21 ; CC1(C)c2ccccc2-c2ccc(N(c3ccc4oc5ccccc5c4c3-c3ccc4oc5ccccc5c4c3)c3ccc(-c4ccccc4)c4ccccc34)cc21 ; CC1(C)c2ccccc2-c2ccc(N(c3ccc4ccccc4c3)c3ccc4oc5ccccc5c4c3-c3ccc4oc5ccccc5c4c3)cc21 ; CC1(C)c2ccccc2-c2ccc(N(c3ccc4cc(-c5ccccc5)ccc4c3)c3ccc4oc5ccccc5c4c3-c3ccc4oc5ccccc5c4c3)cc21]

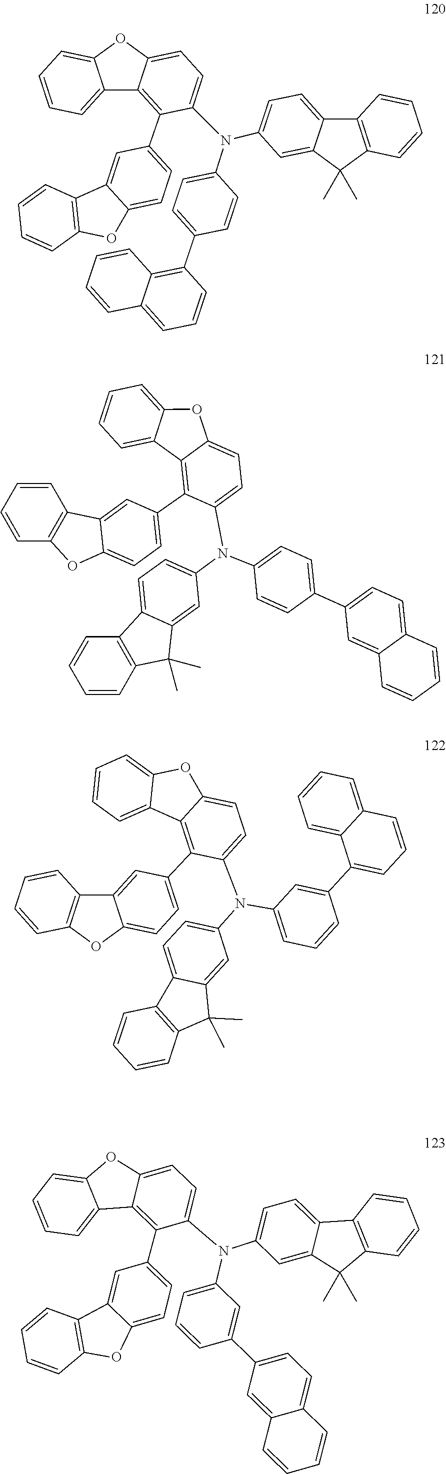 [SMILES: CC1(C)c2ccccc2-c2ccc(N(c3ccc(-c4cccc5ccccc45)cc3)c3ccc4oc5ccccc5c4c3-c3ccc4oc5ccccc5c4c3)cc21 ; CC1(C)c2ccccc2-c2ccc(N(c3ccc(-c4ccc5ccccc5c4)cc3)c3ccc4oc5ccccc5c4c3-c3ccc4oc5ccccc5c4c3)cc21 ; CC1(C)c2ccccc2-c2ccc(N(c3cccc(-c4cccc5ccccc45)c3)c3ccc4oc5ccccc5c4c3-c3ccc4oc5ccccc5c4c3)cc21 ; CC1(C)c2ccccc2-c2ccc(N(c3cccc(-c4ccc5ccccc5c4)c3)c3ccc4oc5ccccc5c4c3-c3ccc4oc5ccccc5c4c3)cc21]

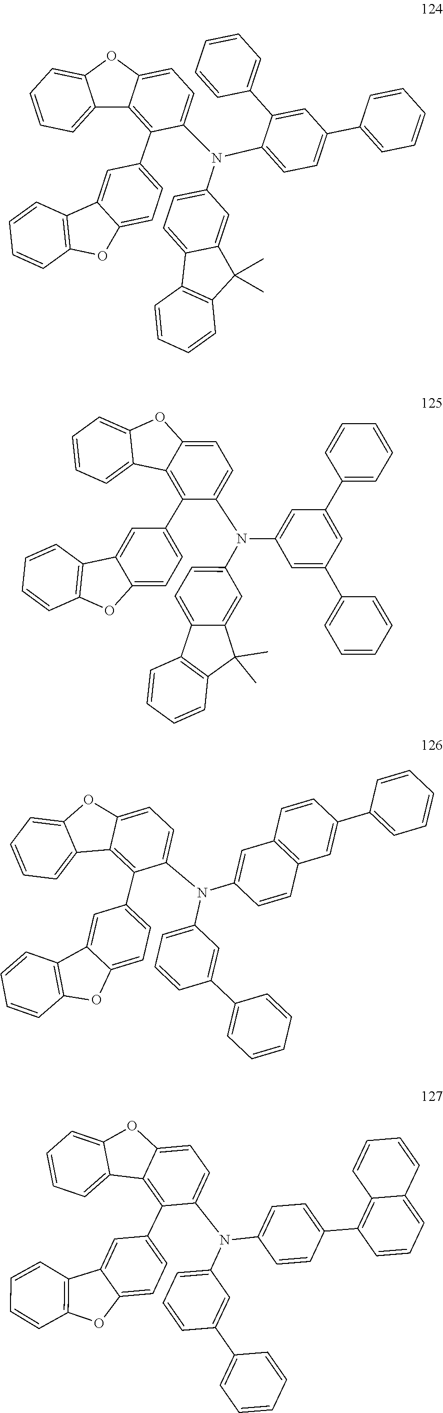 [SMILES: CC1(C)c2ccccc2-c2ccc(N(c3ccc(-c4ccccc4)cc3-c3ccccc3)c3ccc4oc5ccccc5c4c3-c3ccc4oc5ccccc5c4c3)cc21 ; CC1(C)c2ccccc2-c2ccc(N(c3cc(-c4ccccc4)cc(-c4ccccc4)c3)c3ccc4oc5ccccc5c4c3-c3ccc4oc5ccccc5c4c3)cc21 ; c1ccc(-c2cccc(N(c3ccc4cc(-c5ccccc5)ccc4c3)c3ccc4oc5ccccc5c4c3-c3ccc4oc5ccccc5c4c3)c2)cc1 ; c1ccc(-c2cccc(N(c3ccc(-c4cccc5ccccc45)cc3)c3ccc4oc5ccccc5c4c3-c3ccc4oc5ccccc5c4c3)c2)cc1]

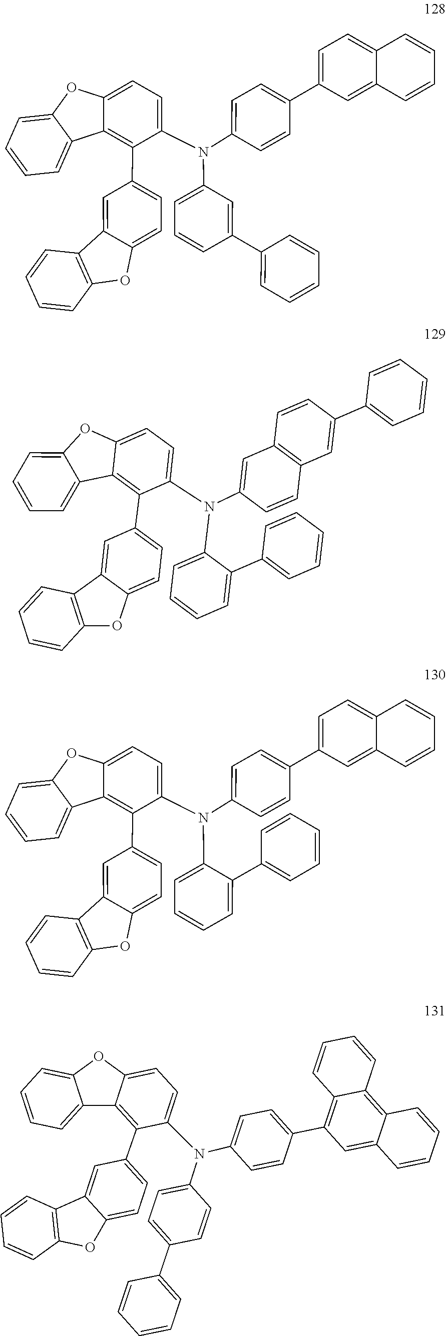 [SMILES: c1ccc(-c2cccc(N(c3ccc(-c4ccc5ccccc5c4)cc3)c3ccc4oc5ccccc5c4c3-c3ccc4oc5ccccc5c4c3)c2)cc1 ; c1ccc(-c2ccc3cc(N(c4ccccc4-c4ccccc4)c4ccc5oc6ccccc6c5c4-c4ccc5oc6ccccc6c5c4)ccc3c2)cc1 ; c1ccc(-c2ccccc2N(c2ccc(-c3ccc4ccccc4c3)cc2)c2ccc3oc4ccccc4c3c2-c2ccc3oc4ccccc4c3c2)cc1 ; c1ccc(-c2ccc(N(c3ccc(-c4cc5ccccc5c5ccccc45)cc3)c3ccc4oc5ccccc5c4c3-c3ccc4oc5ccccc5c4c3)cc2)cc1]

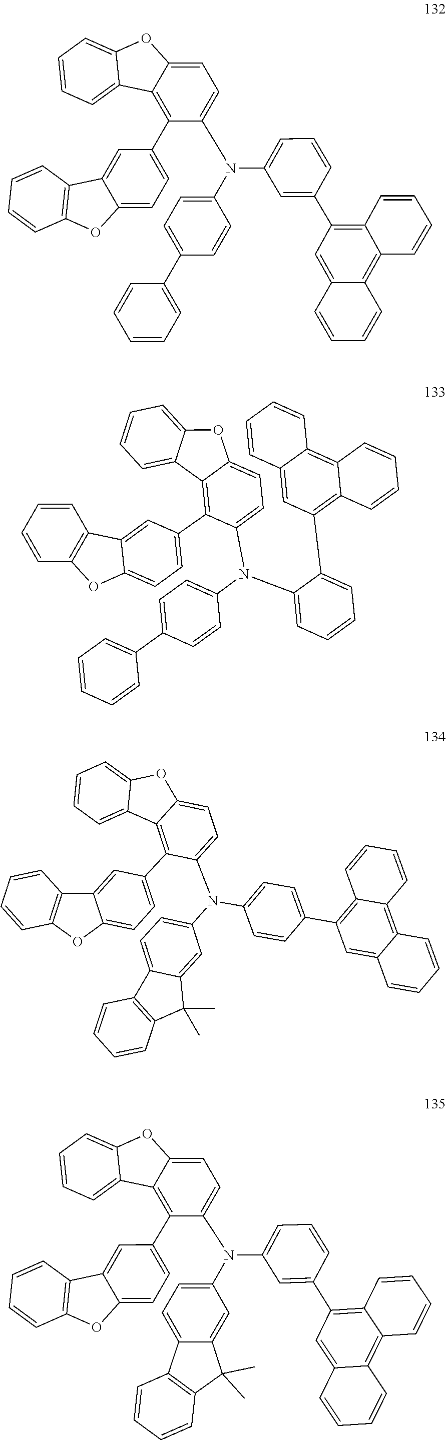 [SMILES: c1ccc(-c2ccc(N(c3cccc(-c4cc5ccccc5c5ccccc45)c3)c3ccc4oc5ccccc5c4c3-c3ccc4oc5ccccc5c4c3)cc2)cc1 ; c1ccc(-c2ccc(N(c3ccccc3-c3cc4ccccc4c4ccccc34)c3ccc4oc5ccccc5c4c3-c3ccc4oc5ccccc5c4c3)cc2)cc1 ; CC1(C)c2ccccc2-c2ccc(N(c3ccc(-c4cc5ccccc5c5ccccc45)cc3)c3ccc4oc5ccccc5c4c3-c3ccc4oc5ccccc5c4c3)cc21 ; CC1(C)c2ccccc2-c2ccc(N(c3cccc(-c4cc5ccccc5c5ccccc45)c3)c3ccc4oc5ccccc5c4c3-c3ccc4oc5ccccc5c4c3)cc21]

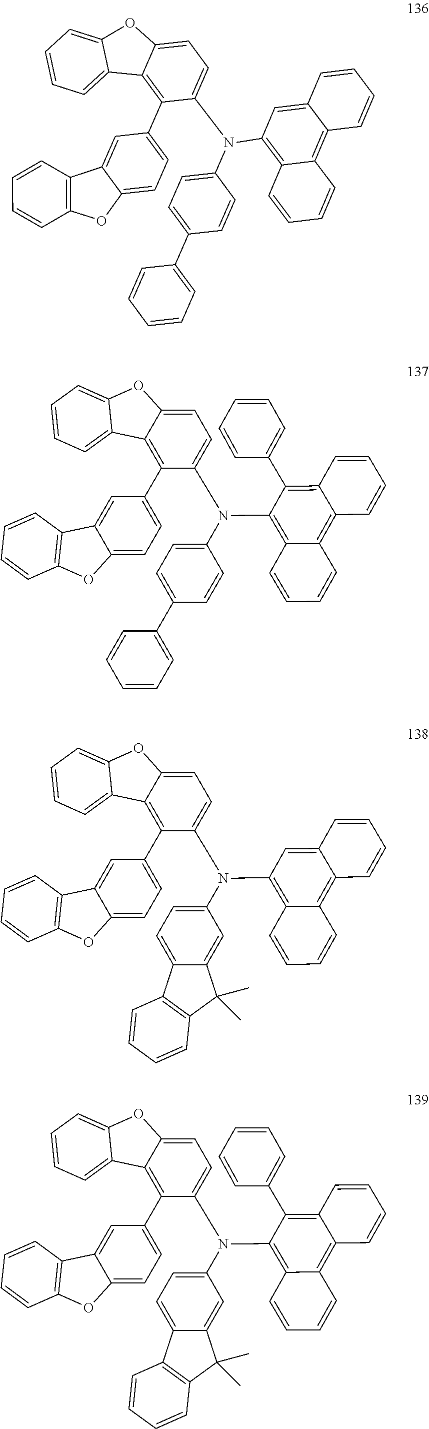 [SMILES: c1ccc(-c2ccc(N(c3ccc4oc5ccccc5c4c3-c3ccc4oc5ccccc5c4c3)c3cc4ccccc4c4ccccc34)cc2)cc1 ; c1ccc(-c2ccc(N(c3ccc4oc5ccccc5c4c3-c3ccc4oc5ccccc5c4c3)c3c(-c4ccccc4)c4ccccc4c4ccccc34)cc2)cc1 ; CC1(C)c2ccccc2-c2ccc(N(c3ccc4oc5ccccc5c4c3-c3ccc4oc5ccccc5c4c3)c3cc4ccccc4c4ccccc34)cc21 ; CC1(C)c2ccccc2-c2ccc(N(c3ccc4oc5ccccc5c4c3-c3ccc4oc5ccccc5c4c3)c3c(-c4ccccc4)c4ccccc4c4ccccc34)cc21]

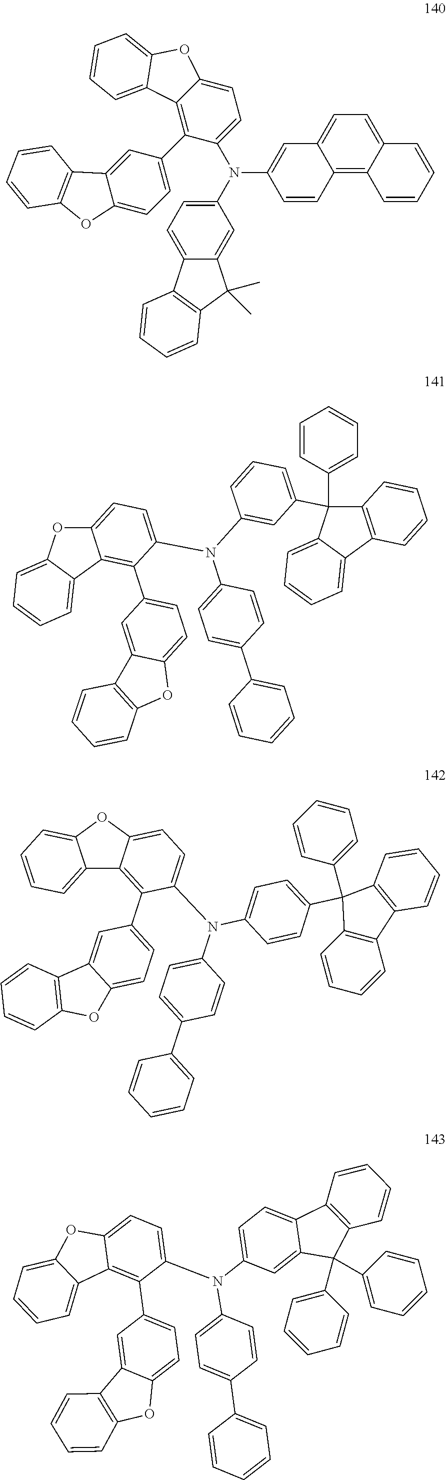 [SMILES: CC1(C)c2ccccc2-c2ccc(N(c3ccc4c(ccc5ccccc54)c3)c3ccc4oc5ccccc5c4c3-c3ccc4oc5ccccc5c4c3)cc21 ; c1ccc(-c2ccc(N(c3cccc(C4(c5ccccc5)c5ccccc5-c5ccccc54)c3)c3ccc4oc5ccccc5c4c3-c3ccc4oc5ccccc5c4c3)cc2)cc1 ; c1ccc(-c2ccc(N(c3ccc(C4(c5ccccc5)c5ccccc5-c5ccccc54)cc3)c3ccc4oc5ccccc5c4c3-c3ccc4oc5ccccc5c4c3)cc2)cc1 ; c1ccc(-c2ccc(N(c3ccc4c(c3)C(c3ccccc3)(c3ccccc3)c3ccccc3-4)c3ccc4oc5ccccc5c4c3-c3ccc4oc5ccccc5c4c3)cc2)cc1]

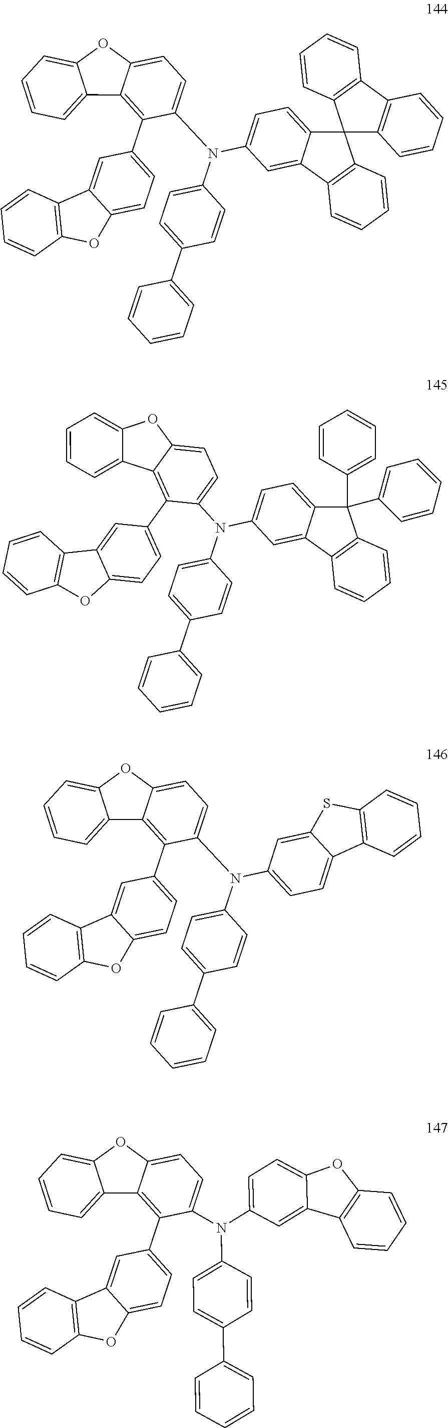 [SMILES: c1ccc(-c2ccc(N(c3ccc4c(c3)-c3ccccc3C43c4ccccc4-c4ccccc43)c3ccc4oc5ccccc5c4c3-c3ccc4oc5ccccc5c4c3)cc2)cc1 ; c1ccc(-c2ccc(N(c3ccc4c(c3)-c3ccccc3C4(c3ccccc3)c3ccccc3)c3ccc4oc5ccccc5c4c3-c3ccc4oc5ccccc5c4c3)cc2)cc1 ; c1ccc(-c2ccc(N(c3ccc4c(c3)sc3ccccc34)c3ccc4oc5ccccc5c4c3-c3ccc4oc5ccccc5c4c3)cc2)cc1 ; c1ccc(-c2ccc(N(c3ccc4oc5ccccc5c4c3)c3ccc4oc5ccccc5c4c3-c3ccc4oc5ccccc5c4c3)cc2)cc1]

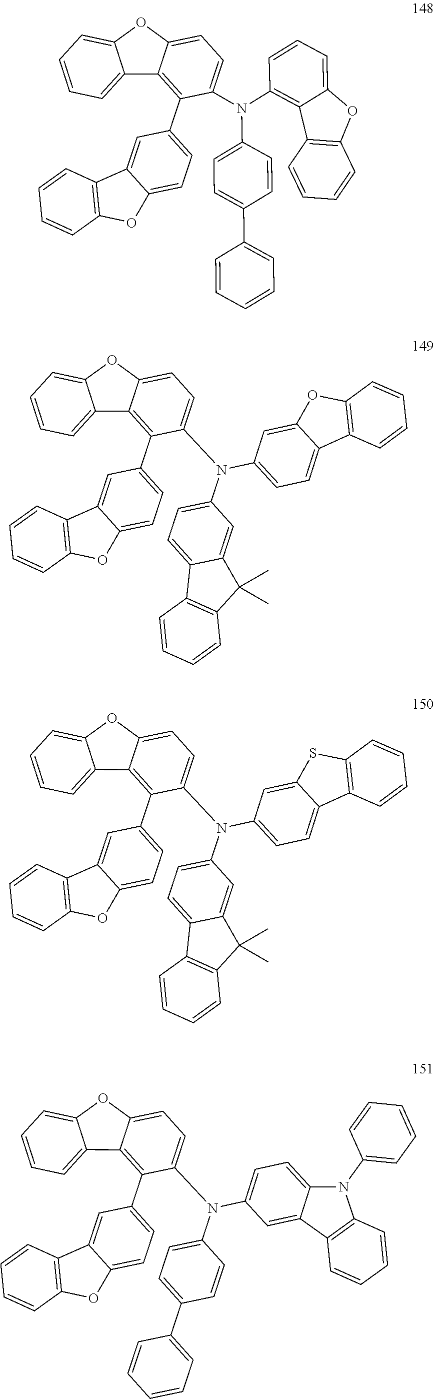 [SMILES: c1ccc(-c2ccc(N(c3ccc4oc5ccccc5c4c3-c3ccc4oc5ccccc5c4c3)c3cccc4oc5ccccc5c34)cc2)cc1 ; CC1(C)c2ccccc2-c2ccc(N(c3ccc4c(c3)oc3ccccc34)c3ccc4oc5ccccc5c4c3-c3ccc4oc5ccccc5c4c3)cc21 ; CC1(C)c2ccccc2-c2ccc(N(c3ccc4c(c3)sc3ccccc34)c3ccc4oc5ccccc5c4c3-c3ccc4oc5ccccc5c4c3)cc21 ; c1ccc(-c2ccc(N(c3ccc4c(c3)c3ccccc3n4-c3ccccc3)c3ccc4oc5ccccc5c4c3-c3ccc4oc5ccccc5c4c3)cc2)cc1]

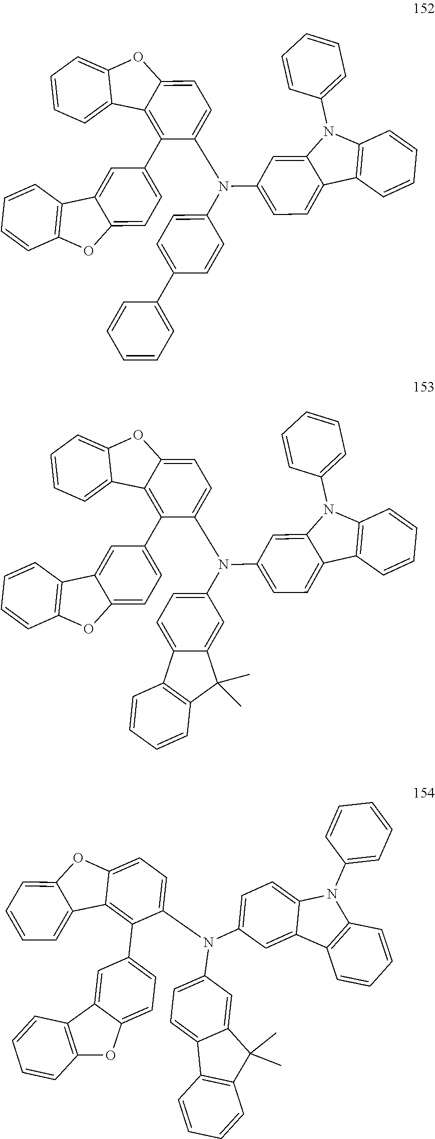 [SMILES: c1ccc(-c2ccc(N(c3ccc4c5ccccc5n(-c5ccccc5)c4c3)c3ccc4oc5ccccc5c4c3-c3ccc4oc5ccccc5c4c3)cc2)cc1 ; CC1(C)c2ccccc2-c2ccc(N(c3ccc4c5ccccc5n(-c5ccccc5)c4c3)c3ccc4oc5ccccc5c4c3-c3ccc4oc5ccccc5c4c3)cc21 ; CC1(C)c2ccccc2-c2ccc(N(c3ccc4c(c3)c3ccccc3n4-c3ccccc3)c3ccc4oc5ccccc5c4c3-c3ccc4oc5ccccc5c4c3)cc21]

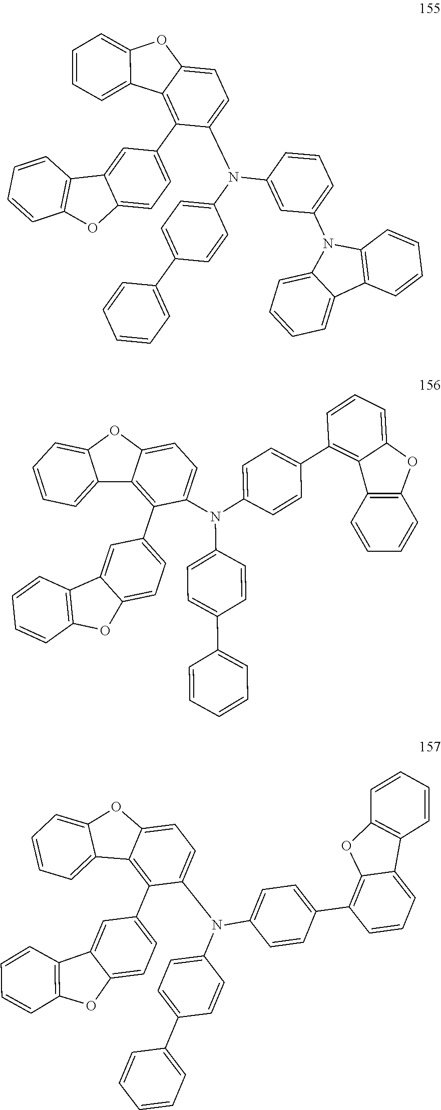 [SMILES: c1ccc(-c2ccc(N(c3cccc(-n4c5ccccc5c5ccccc54)c3)c3ccc4oc5ccccc5c4c3-c3ccc4oc5ccccc5c4c3)cc2)cc1 ; c1ccc(-c2ccc(N(c3ccc(-c4cccc5oc6ccccc6c45)cc3)c3ccc4oc5ccccc5c4c3-c3ccc4oc5ccccc5c4c3)cc2)cc1 ; c1ccc(-c2ccc(N(c3ccc(-c4cccc5c4oc4ccccc45)cc3)c3ccc4oc5ccccc5c4c3-c3ccc4oc5ccccc5c4c3)cc2)cc1]

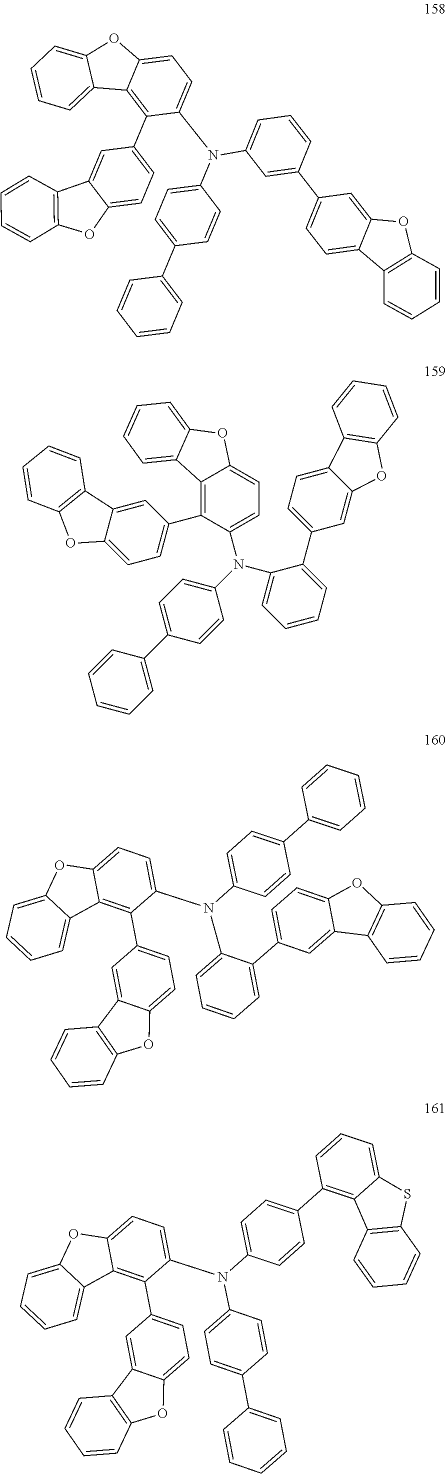 [SMILES: c1ccc(-c2ccc(N(c3cccc(-c4ccc5c(c4)oc4ccccc45)c3)c3ccc4oc5ccccc5c4c3-c3ccc4oc5ccccc5c4c3)cc2)cc1 ; c1ccc(-c2ccc(N(c3ccccc3-c3ccc4c(c3)oc3ccccc34)c3ccc4oc5ccccc5c4c3-c3ccc4oc5ccccc5c4c3)cc2)cc1 ; c1ccc(-c2ccc(N(c3ccccc3-c3ccc4oc5ccccc5c4c3)c3ccc4oc5ccccc5c4c3-c3ccc4oc5ccccc5c4c3)cc2)cc1 ; c1ccc(-c2ccc(N(c3ccc(-c4cccc5sc6ccccc6c45)cc3)c3ccc4oc5ccccc5c4c3-c3ccc4oc5ccccc5c4c3)cc2)cc1]

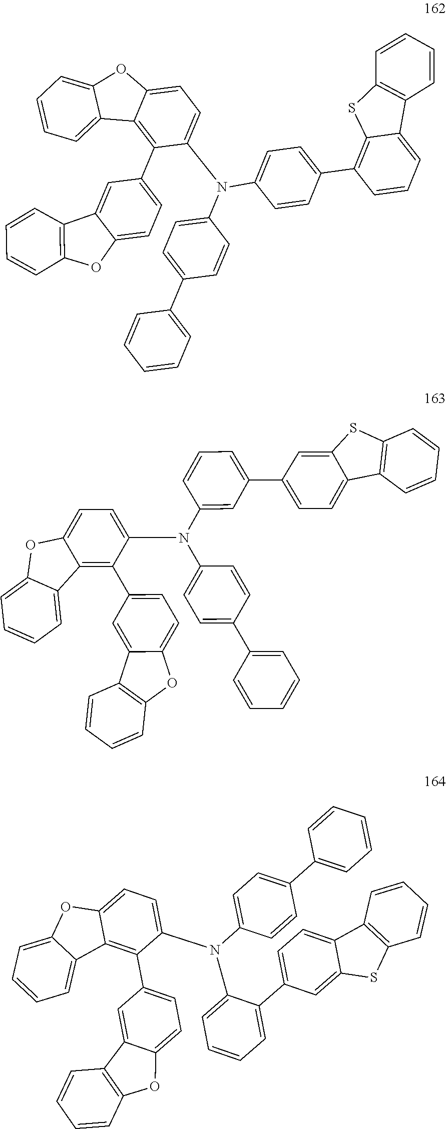 [SMILES: c1ccc(-c2ccc(N(c3ccc(-c4cccc5c4sc4ccccc45)cc3)c3ccc4oc5ccccc5c4c3-c3ccc4oc5ccccc5c4c3)cc2)cc1 ; c1ccc(-c2ccc(N(c3cccc(-c4ccc5c(c4)sc4ccccc45)c3)c3ccc4oc5ccccc5c4c3-c3ccc4oc5ccccc5c4c3)cc2)cc1 ; c1ccc(-c2ccc(N(c3ccccc3-c3ccc4c(c3)sc3ccccc34)c3ccc4oc5ccccc5c4c3-c3ccc4oc5ccccc5c4c3)cc2)cc1]

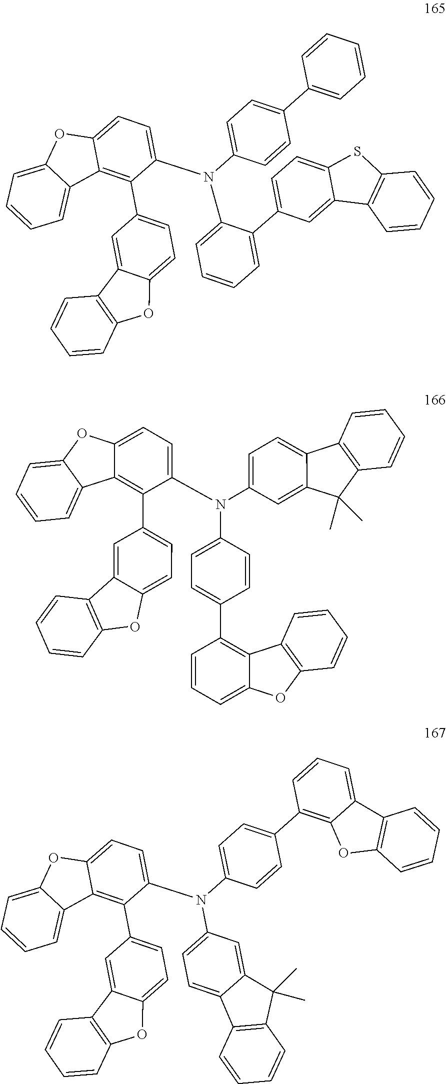 [SMILES: c1ccc(-c2ccc(N(c3ccccc3-c3ccc4sc5ccccc5c4c3)c3ccc4oc5ccccc5c4c3-c3ccc4oc5ccccc5c4c3)cc2)cc1 ; CC1(C)c2ccccc2-c2ccc(N(c3ccc(-c4cccc5oc6ccccc6c45)cc3)c3ccc4oc5ccccc5c4c3-c3ccc4oc5ccccc5c4c3)cc21 ; CC1(C)c2ccccc2-c2ccc(N(c3ccc(-c4cccc5c4oc4ccccc45)cc3)c3ccc4oc5ccccc5c4c3-c3ccc4oc5ccccc5c4c3)cc21]

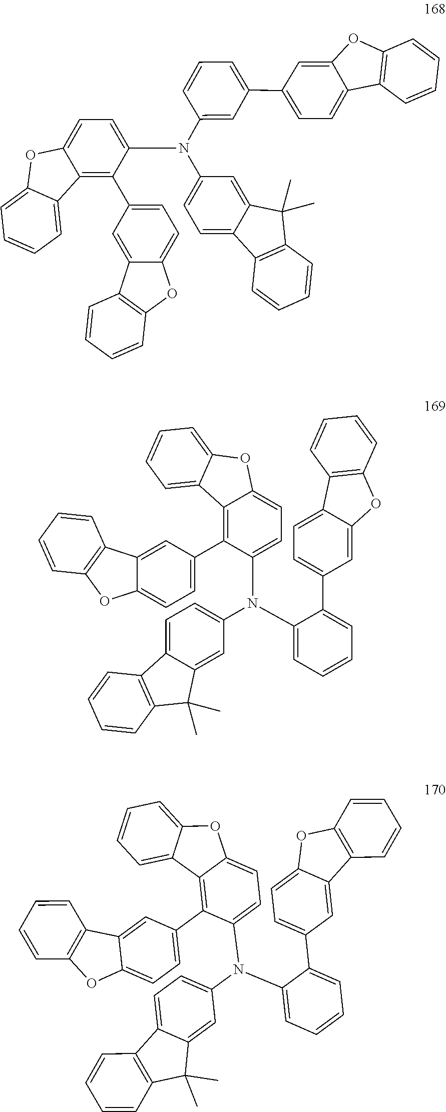 [SMILES: CC1(C)c2ccccc2-c2ccc(N(c3cccc(-c4ccc5c(c4)oc4ccccc45)c3)c3ccc4oc5ccccc5c4c3-c3ccc4oc5ccccc5c4c3)cc21 ; CC1(C)c2ccccc2-c2ccc(N(c3ccccc3-c3ccc4c(c3)oc3ccccc34)c3ccc4oc5ccccc5c4c3-c3ccc4oc5ccccc5c4c3)cc21 ; CC1(C)c2ccccc2-c2ccc(N(c3ccccc3-c3ccc4oc5ccccc5c4c3)c3ccc4oc5ccccc5c4c3-c3ccc4oc5ccccc5c4c3)cc21]

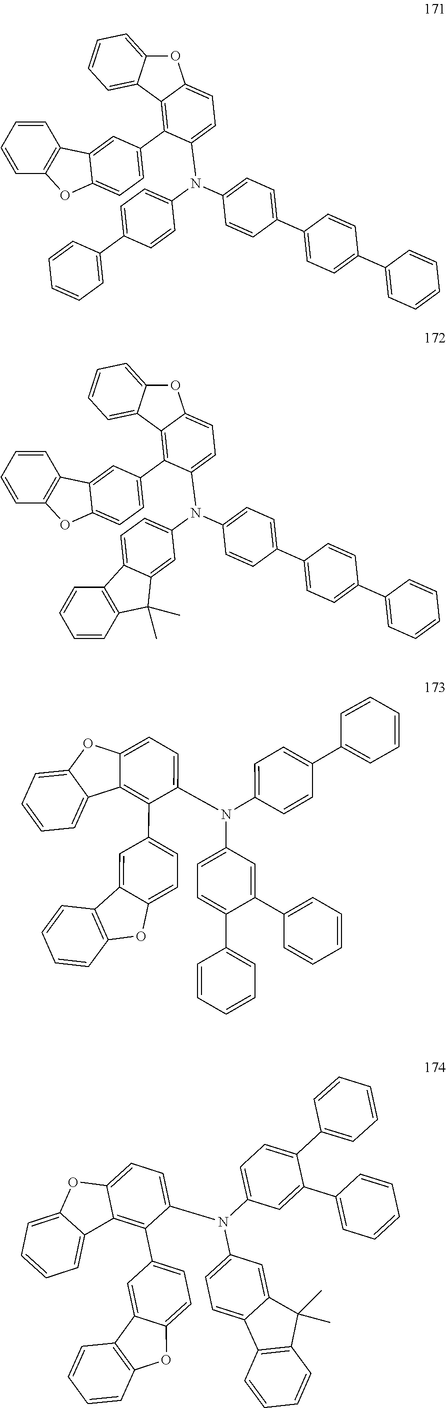 [SMILES: c1ccc(-c2ccc(-c3ccc(N(c4ccc(-c5ccccc5)cc4)c4ccc5oc6ccccc6c5c4-c4ccc5oc6ccccc6c5c4)cc3)cc2)cc1 ; CC1(C)c2ccccc2-c2ccc(N(c3ccc(-c4ccc(-c5ccccc5)cc4)cc3)c3ccc4oc5ccccc5c4c3-c3ccc4oc5ccccc5c4c3)cc21 ; c1ccc(-c2ccc(N(c3ccc(-c4ccccc4)c(-c4ccccc4)c3)c3ccc4oc5ccccc5c4c3-c3ccc4oc5ccccc5c4c3)cc2)cc1 ; CC1(C)c2ccccc2-c2ccc(N(c3ccc(-c4ccccc4)c(-c4ccccc4)c3)c3ccc4oc5ccccc5c4c3-c3ccc4oc5ccccc5c4c3)cc21]

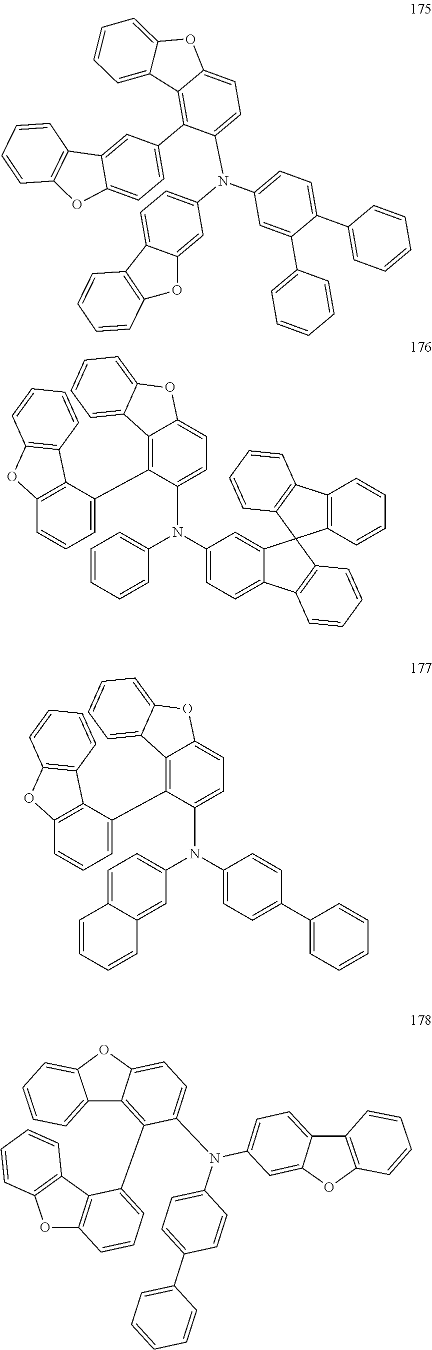

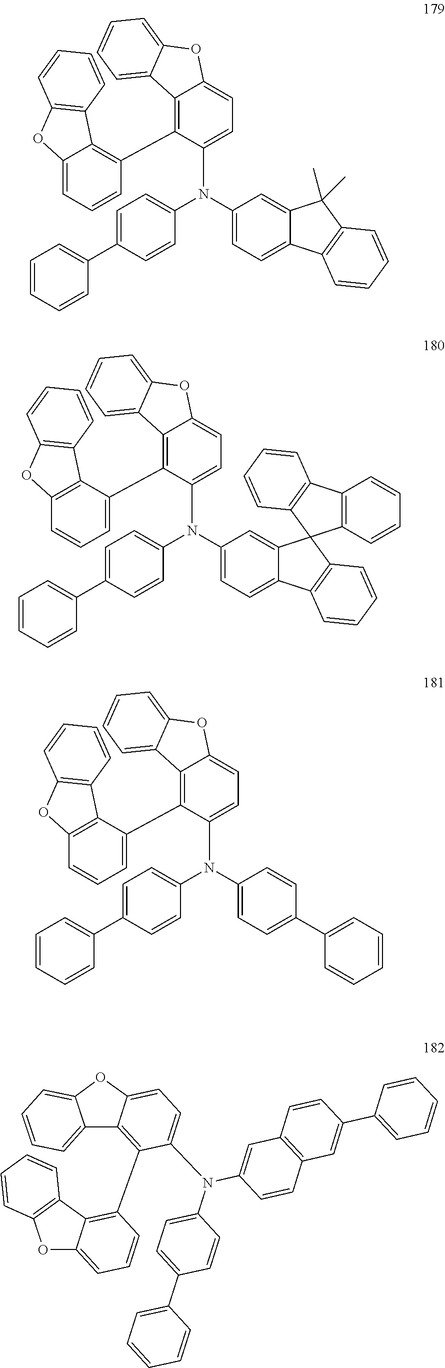 [SMILES: CC1(C)c2ccccc2-c2ccc(N(c3ccc(-c4ccccc4)cc3)c3ccc4oc5ccccc5c4c3-c3cccc4oc5ccccc5c34)cc21 ; c1ccc(-c2ccc(N(c3ccc4c(c3)C3(c5ccccc5-c5ccccc53)c3ccccc3-4)c3ccc4oc5ccccc5c4c3-c3cccc4oc5ccccc5c34)cc2)cc1 ; c1ccc(-c2ccc(N(c3ccc(-c4ccccc4)cc3)c3ccc4oc5ccccc5c4c3-c3cccc4oc5ccccc5c34)cc2)cc1 ; c1ccc(-c2ccc(N(c3ccc4cc(-c5ccccc5)ccc4c3)c3ccc4oc5ccccc5c4c3-c3cccc4oc5ccccc5c34)cc2)cc1]

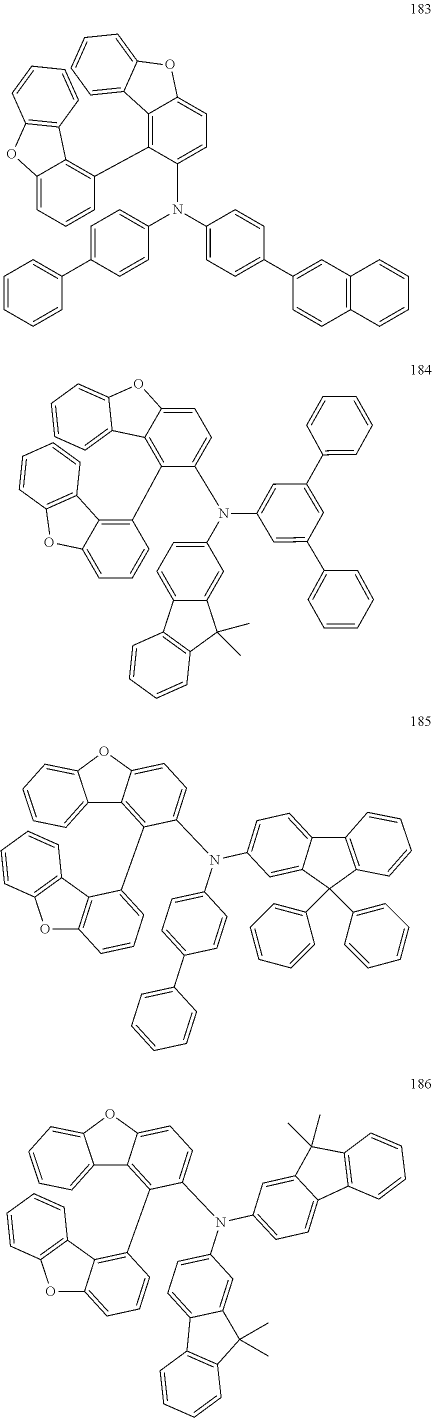 [SMILES: c1ccc(-c2ccc(N(c3ccc(-c4ccc5ccccc5c4)cc3)c3ccc4oc5ccccc5c4c3-c3cccc4oc5ccccc5c34)cc2)cc1 ; CC1(C)c2ccccc2-c2ccc(N(c3cc(-c4ccccc4)cc(-c4ccccc4)c3)c3ccc4oc5ccccc5c4c3-c3cccc4oc5ccccc5c34)cc21 ; c1ccc(-c2ccc(N(c3ccc4c(c3)C(c3ccccc3)(c3ccccc3)c3ccccc3-4)c3ccc4oc5ccccc5c4c3-c3cccc4oc5ccccc5c34)cc2)cc1 ; CC1(C)c2ccccc2-c2ccc(N(c3ccc4c(c3)C(C)(C)c3ccccc3-4)c3ccc4oc5ccccc5c4c3-c3cccc4oc5ccccc5c34)cc21]

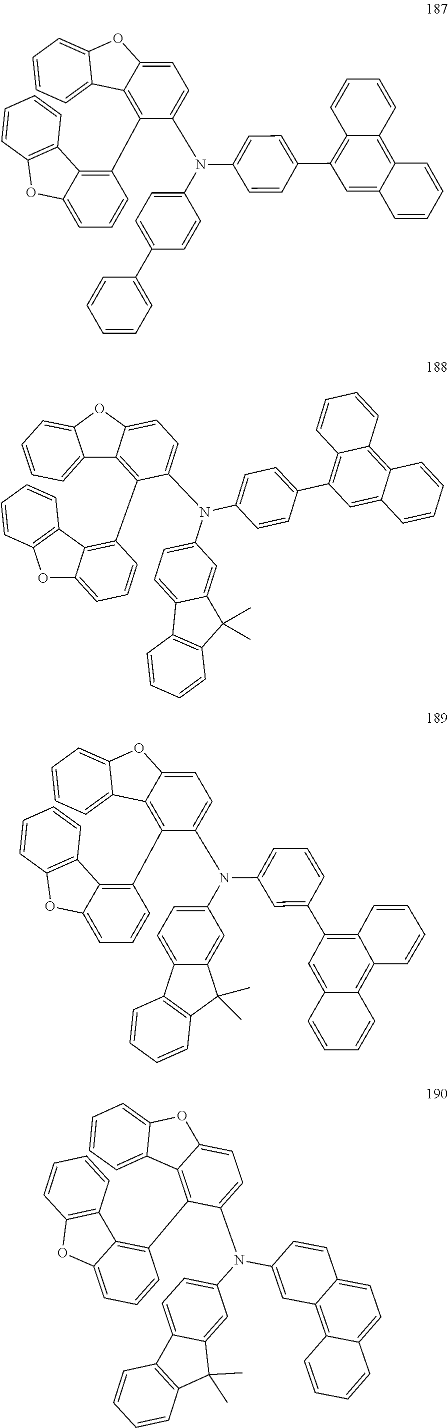 [SMILES: c1ccc(-c2ccc(N(c3ccc(-c4cc5ccccc5c5ccccc45)cc3)c3ccc4oc5ccccc5c4c3-c3cccc4oc5ccccc5c34)cc2)cc1 ; CC1(C)c2ccccc2-c2ccc(N(c3ccc(-c4cc5ccccc5c5ccccc45)cc3)c3ccc4oc5ccccc5c4c3-c3cccc4oc5ccccc5c34)cc21 ; CC1(C)c2ccccc2-c2ccc(N(c3cccc(-c4cc5ccccc5c5ccccc45)c3)c3ccc4oc5ccccc5c4c3-c3cccc4oc5ccccc5c34)cc21 ; CC1(C)c2ccccc2-c2ccc(N(c3ccc4ccc5ccccc5c4c3)c3ccc4oc5ccccc5c4c3-c3cccc4oc5ccccc5c34)cc21]

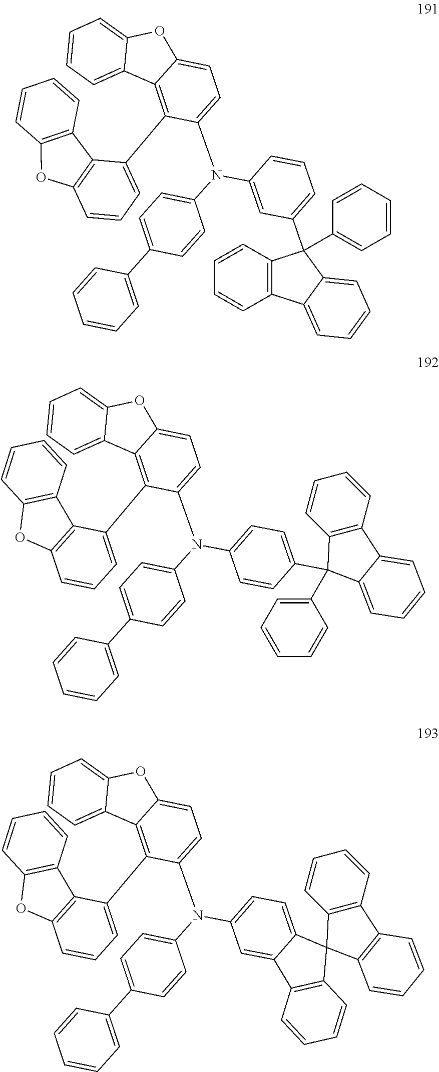 [SMILES: c1ccc(-c2ccc(N(c3cccc(C4(c5ccccc5)c5ccccc5-c5ccccc54)c3)c3ccc4oc5ccccc5c4c3-c3cccc4oc5ccccc5c34)cc2)cc1 ; c1ccc(-c2ccc(N(c3ccc(C4(c5ccccc5)c5ccccc5-c5ccccc54)cc3)c3ccc4oc5ccccc5c4c3-c3cccc4oc5ccccc5c34)cc2)cc1 ; c1ccc(-c2ccc(N(c3ccc4c(c3)-c3ccccc3C43c4ccccc4-c4ccccc43)c3ccc4oc5ccccc5c4c3-c3cccc4oc5ccccc5c34)cc2)cc1]

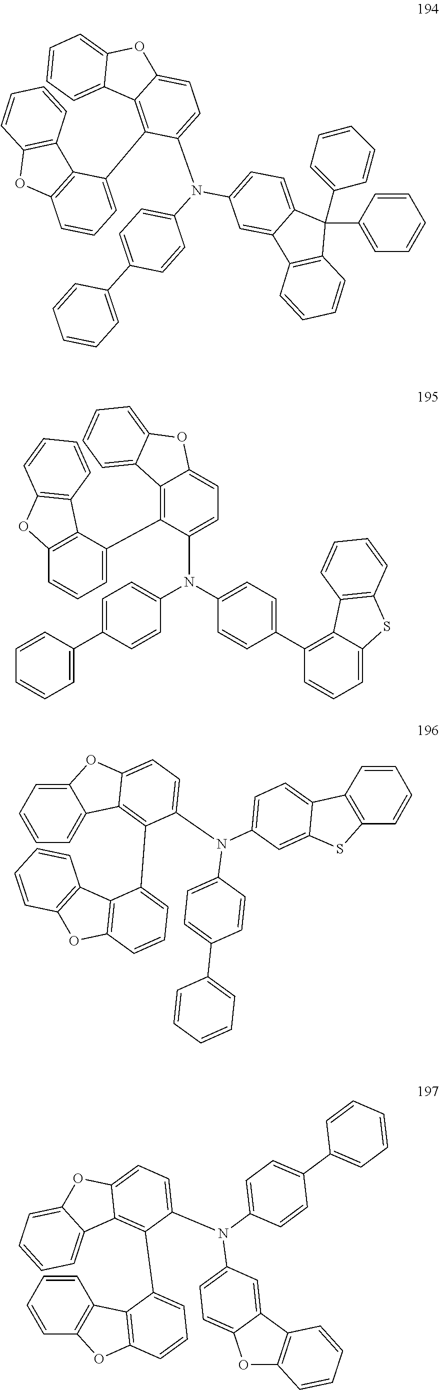 [SMILES: c1ccc(-c2ccc(N(c3ccc4c(c3)-c3ccccc3C4(c3ccccc3)c3ccccc3)c3ccc4oc5ccccc5c4c3-c3cccc4oc5ccccc5c34)cc2)cc1 ; c1ccc(-c2ccc(N(c3ccc(-c4cccc5sc6ccccc6c45)cc3)c3ccc4oc5ccccc5c4c3-c3cccc4oc5ccccc5c34)cc2)cc1 ; c1ccc(-c2ccc(N(c3ccc4c(c3)sc3ccccc34)c3ccc4oc5ccccc5c4c3-c3cccc4oc5ccccc5c34)cc2)cc1 ; c1ccc(-c2ccc(N(c3ccc4oc5ccccc5c4c3)c3ccc4oc5ccccc5c4c3-c3cccc4oc5ccccc5c34)cc2)cc1]

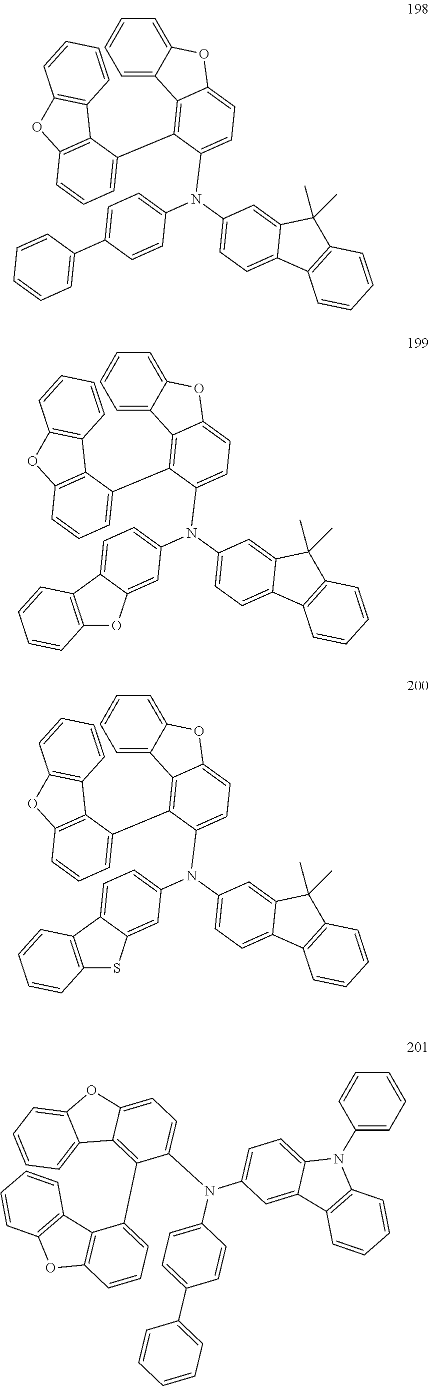 [SMILES: CC1(C)c2ccccc2-c2ccc(N(c3ccc(-c4ccccc4)cc3)c3ccc4oc5ccccc5c4c3-c3cccc4oc5ccccc5c34)cc21 ; CC1(C)c2ccccc2-c2ccc(N(c3ccc4c(c3)oc3ccccc34)c3ccc4oc5ccccc5c4c3-c3cccc4oc5ccccc5c34)cc21 ; CC1(C)c2ccccc2-c2ccc(N(c3ccc4c(c3)sc3ccccc34)c3ccc4oc5ccccc5c4c3-c3cccc4oc5ccccc5c34)cc21 ; c1ccc(-c2ccc(N(c3ccc4c(c3)c3ccccc3n4-c3ccccc3)c3ccc4oc5ccccc5c4c3-c3cccc4oc5ccccc5c34)cc2)cc1]

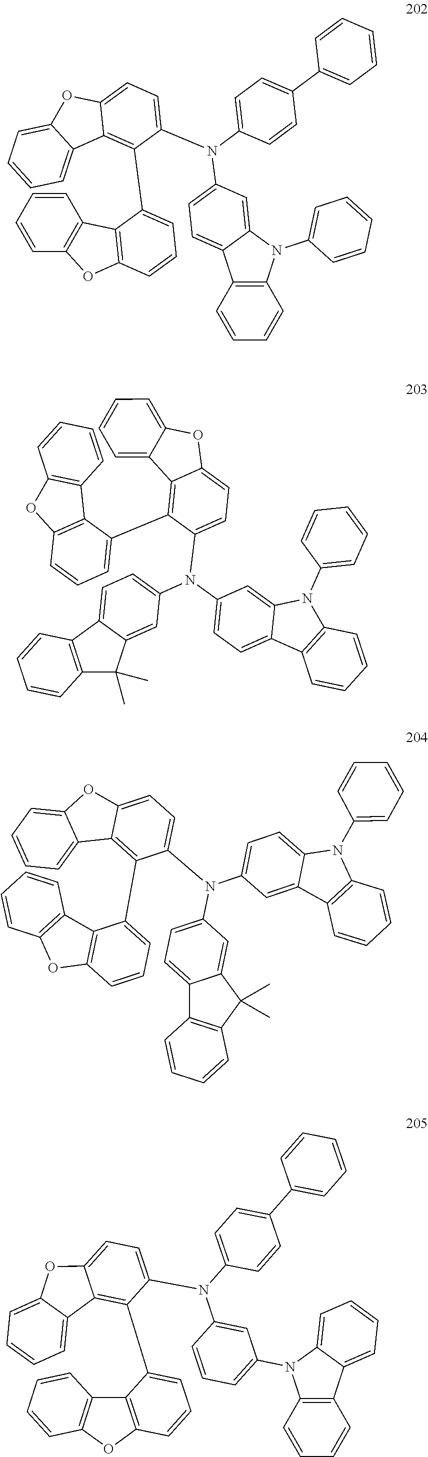 [SMILES: c1ccc(-c2ccc(N(c3ccc4c5ccccc5n(-c5ccccc5)c4c3)c3ccc4oc5ccccc5c4c3-c3cccc4oc5ccccc5c34)cc2)cc1 ; CC1(C)c2ccccc2-c2ccc(N(c3ccc4c5ccccc5n(-c5ccccc5)c4c3)c3ccc4oc5ccccc5c4c3-c3cccc4oc5ccccc5c34)cc21 ; CC1(C)c2ccccc2-c2ccc(N(c3ccc4c(c3)c3ccccc3n4-c3ccccc3)c3ccc4oc5ccccc5c4c3-c3cccc4oc5ccccc5c34)cc21 ; c1ccc(-c2ccc(N(c3cccc(-n4c5ccccc5c5ccccc54)c3)c3ccc4oc5ccccc5c4c3-c3cccc4oc5ccccc5c34)cc2)cc1]

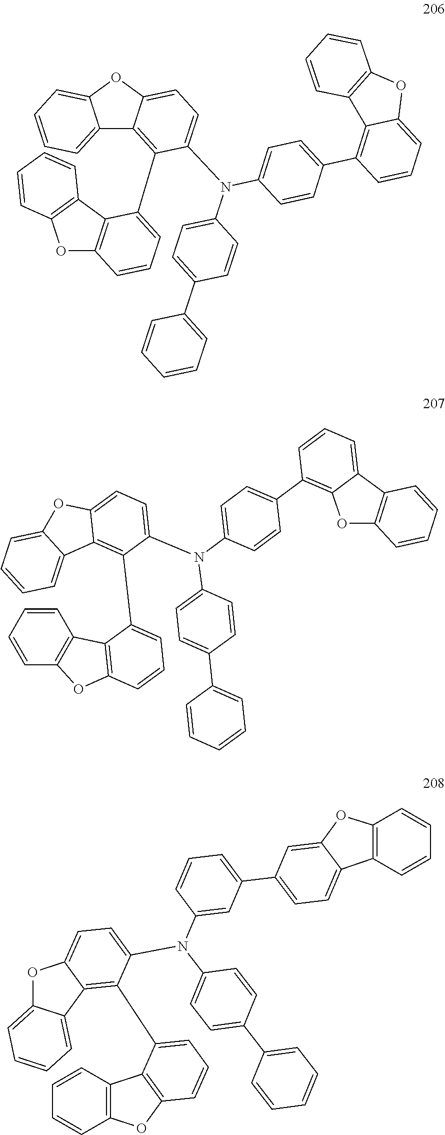 [SMILES: c1ccc(-c2ccc(N(c3ccc(-c4cccc5oc6ccccc6c45)cc3)c3ccc4oc5ccccc5c4c3-c3cccc4oc5ccccc5c34)cc2)cc1 ; c1ccc(-c2ccc(N(c3ccc(-c4cccc5c4oc4ccccc45)cc3)c3ccc4oc5ccccc5c4c3-c3cccc4oc5ccccc5c34)cc2)cc1 ; c1ccc(-c2ccc(N(c3cccc(-c4ccc5c(c4)oc4ccccc45)c3)c3ccc4oc5ccccc5c4c3-c3cccc4oc5ccccc5c34)cc2)cc1]

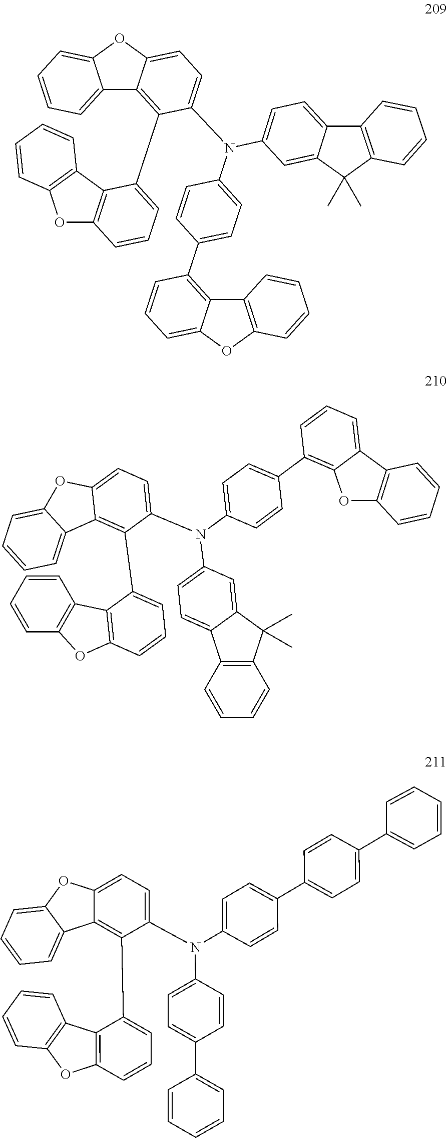 [SMILES: CC1(C)c2ccccc2-c2ccc(N(c3ccc(-c4cccc5oc6ccccc6c45)cc3)c3ccc4oc5ccccc5c4c3-c3cccc4oc5ccccc5c34)cc21 ; CC1(C)c2ccccc2-c2ccc(N(c3ccc(-c4cccc5c4oc4ccccc45)cc3)c3ccc4oc5ccccc5c4c3-c3cccc4oc5ccccc5c34)cc21 ; c1ccc(-c2ccc(-c3ccc(N(c4ccc(-c5ccccc5)cc4)c4ccc5oc6ccccc6c5c4-c4cccc5oc6ccccc6c45)cc3)cc2)cc1]

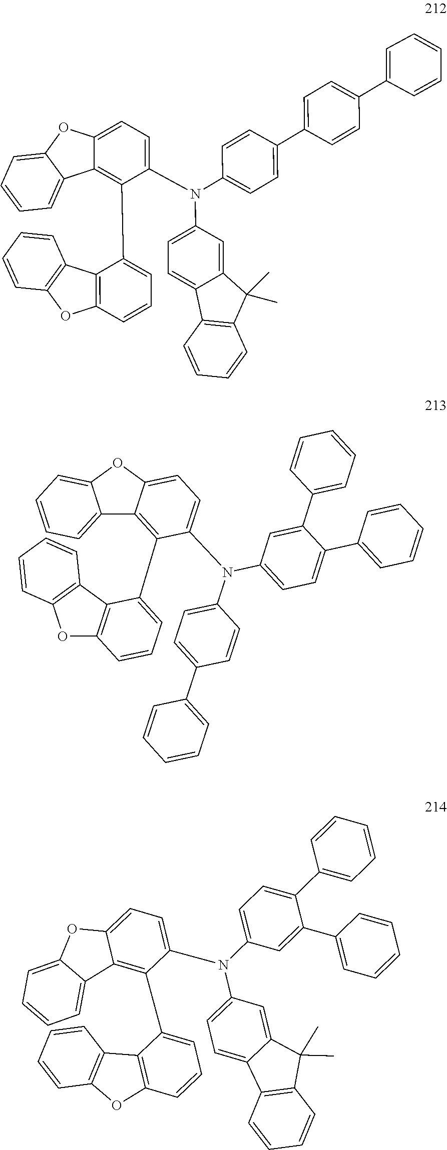 [SMILES: CC1(C)c2ccccc2-c2ccc(N(c3ccc(-c4ccc(-c5ccccc5)cc4)cc3)c3ccc4oc5ccccc5c4c3-c3cccc4oc5ccccc5c34)cc21 ; c1ccc(-c2ccc(N(c3ccc(-c4ccccc4)c(-c4ccccc4)c3)c3ccc4oc5ccccc5c4c3-c3cccc4oc5ccccc5c34)cc2)cc1 ; CC1(C)c2ccccc2-c2ccc(N(c3ccc(-c4ccccc4)c(-c4ccccc4)c3)c3ccc4oc5ccccc5c4c3-c3cccc4oc5ccccc5c34)cc21]

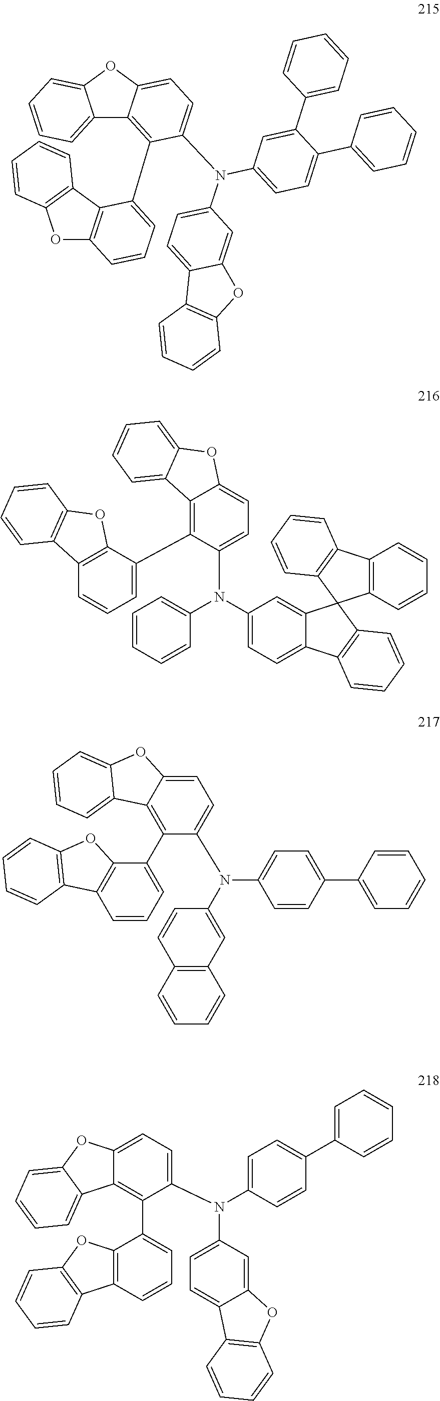 [SMILES: c1ccc(-c2ccc(N(c3ccc4c(c3)oc3ccccc34)c3ccc4oc5ccccc5c4c3-c3cccc4oc5ccccc5c34)cc2-c2ccccc2)cc1 ; c1ccc(N(c2ccc3c(c2)C2(c4ccccc4-c4ccccc42)c2ccccc2-3)c2ccc3oc4ccccc4c3c2-c2cccc3c2oc2ccccc23)cc1 ; c1ccc(-c2ccc(N(c3ccc4ccccc4c3)c3ccc4oc5ccccc5c4c3-c3cccc4c3oc3ccccc34)cc2)cc1 ; c1ccc(-c2ccc(N(c3ccc4c(c3)oc3ccccc34)c3ccc4oc5ccccc5c4c3-c3cccc4c3oc3ccccc34)cc2)cc1]

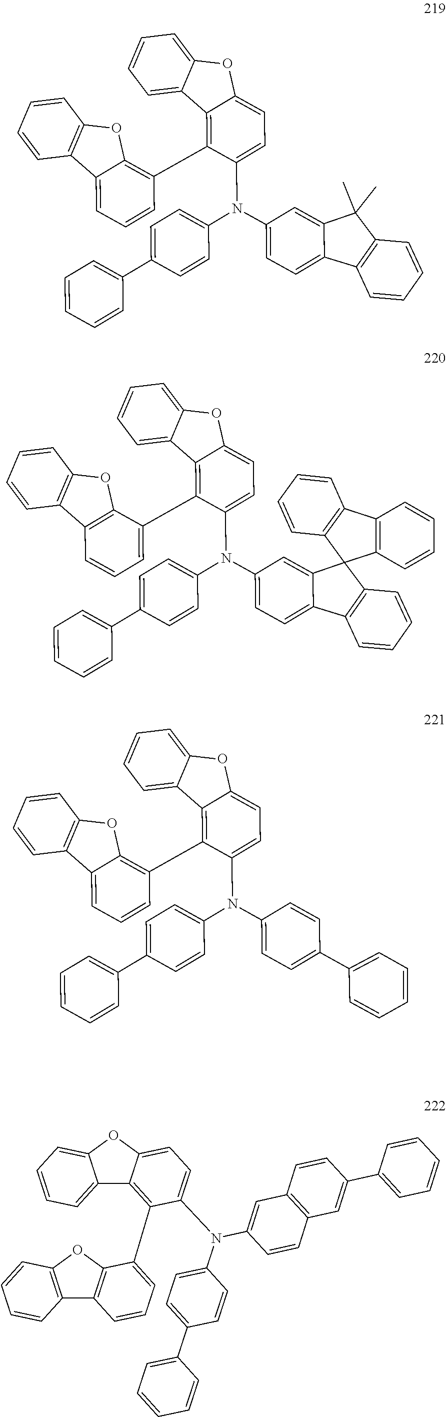 [SMILES: CC1(C)c2ccccc2-c2ccc(N(c3ccc(-c4ccccc4)cc3)c3ccc4oc5ccccc5c4c3-c3cccc4c3oc3ccccc34)cc21 ; c1ccc(-c2ccc(N(c3ccc4c(c3)C3(c5ccccc5-c5ccccc53)c3ccccc3-4)c3ccc4oc5ccccc5c4c3-c3cccc4c3oc3ccccc34)cc2)cc1 ; c1ccc(-c2ccc(N(c3ccc(-c4ccccc4)cc3)c3ccc4oc5ccccc5c4c3-c3cccc4c3oc3ccccc34)cc2)cc1 ; c1ccc(-c2ccc(N(c3ccc4cc(-c5ccccc5)ccc4c3)c3ccc4oc5ccccc5c4c3-c3cccc4c3oc3ccccc34)cc2)cc1]

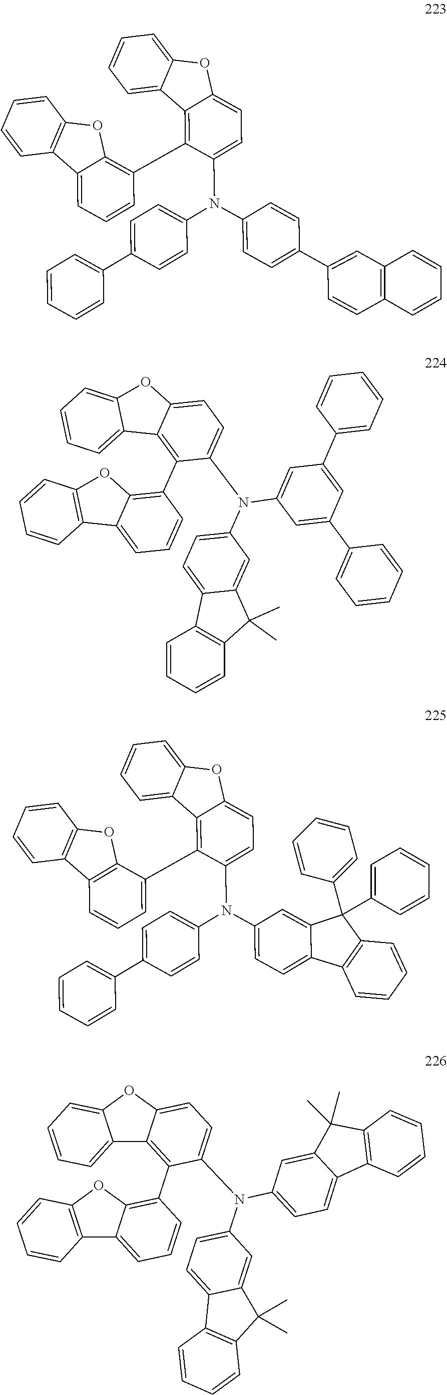 [SMILES: c1ccc(-c2ccc(N(c3ccc(-c4ccc5ccccc5c4)cc3)c3ccc4oc5ccccc5c4c3-c3cccc4c3oc3ccccc34)cc2)cc1 ; CC1(C)c2ccccc2-c2ccc(N(c3cc(-c4ccccc4)cc(-c4ccccc4)c3)c3ccc4oc5ccccc5c4c3-c3cccc4c3oc3ccccc34)cc21 ; c1ccc(-c2ccc(N(c3ccc4c(c3)C(c3ccccc3)(c3ccccc3)c3ccccc3-4)c3ccc4oc5ccccc5c4c3-c3cccc4c3oc3ccccc34)cc2)cc1 ; CC1(C)c2ccccc2-c2ccc(N(c3ccc4c(c3)C(C)(C)c3ccccc3-4)c3ccc4oc5ccccc5c4c3-c3cccc4c3oc3ccccc34)cc21]

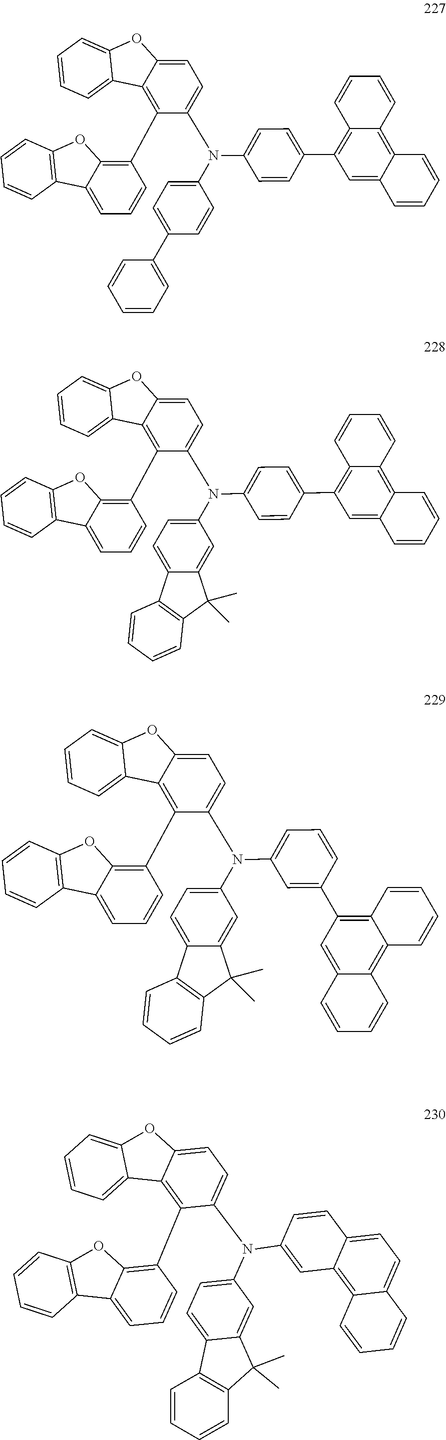 [SMILES: c1ccc(-c2ccc(N(c3ccc(-c4cc5ccccc5c5ccccc45)cc3)c3ccc4oc5ccccc5c4c3-c3cccc4c3oc3ccccc34)cc2)cc1 ; CC1(C)c2ccccc2-c2ccc(N(c3ccc(-c4cc5ccccc5c5ccccc45)cc3)c3ccc4oc5ccccc5c4c3-c3cccc4c3oc3ccccc34)cc21 ; CC1(C)c2ccccc2-c2ccc(N(c3cccc(-c4cc5ccccc5c5ccccc45)c3)c3ccc4oc5ccccc5c4c3-c3cccc4c3oc3ccccc34)cc21 ; CC1(C)c2ccccc2-c2ccc(N(c3ccc4ccc5ccccc5c4c3)c3ccc4oc5ccccc5c4c3-c3cccc4c3oc3ccccc34)cc21]

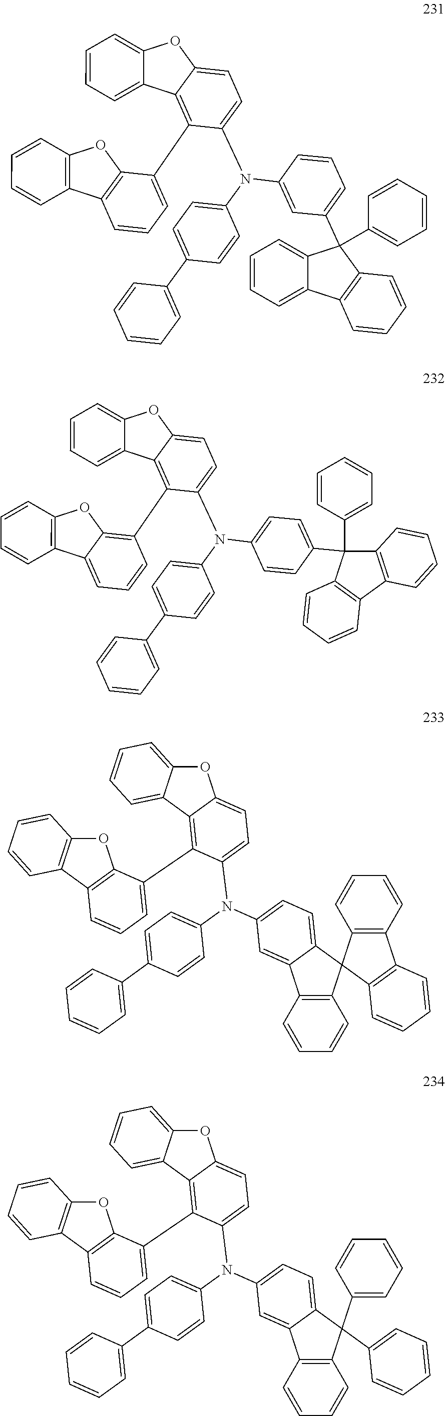 [SMILES: c1ccc(-c2ccc(N(c3cccc(C4(c5ccccc5)c5ccccc5-c5ccccc54)c3)c3ccc4oc5ccccc5c4c3-c3cccc4c3oc3ccccc34)cc2)cc1 ; c1ccc(-c2ccc(N(c3ccc(C4(c5ccccc5)c5ccccc5-c5ccccc54)cc3)c3ccc4oc5ccccc5c4c3-c3cccc4c3oc3ccccc34)cc2)cc1 ; c1ccc(-c2ccc(N(c3ccc4c(c3)-c3ccccc3C43c4ccccc4-c4ccccc43)c3ccc4oc5ccccc5c4c3-c3cccc4c3oc3ccccc34)cc2)cc1 ; c1ccc(-c2ccc(N(c3ccc4c(c3)-c3ccccc3C4(c3ccccc3)c3ccccc3)c3ccc4oc5ccccc5c4c3-c3cccc4c3oc3ccccc34)cc2)cc1]

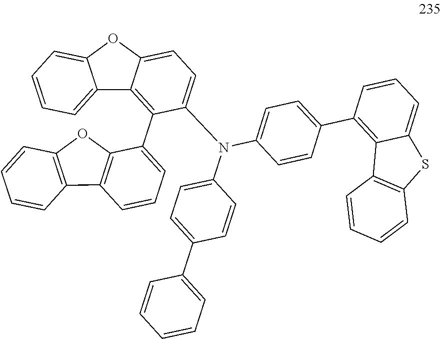 [SMILES: c1ccc(-c2ccc(N(c3ccc(-c4cccc5sc6ccccc6c45)cc3)c3ccc4oc5ccccc5c4c3-c3cccc4c3oc3ccccc34)cc2)cc1]

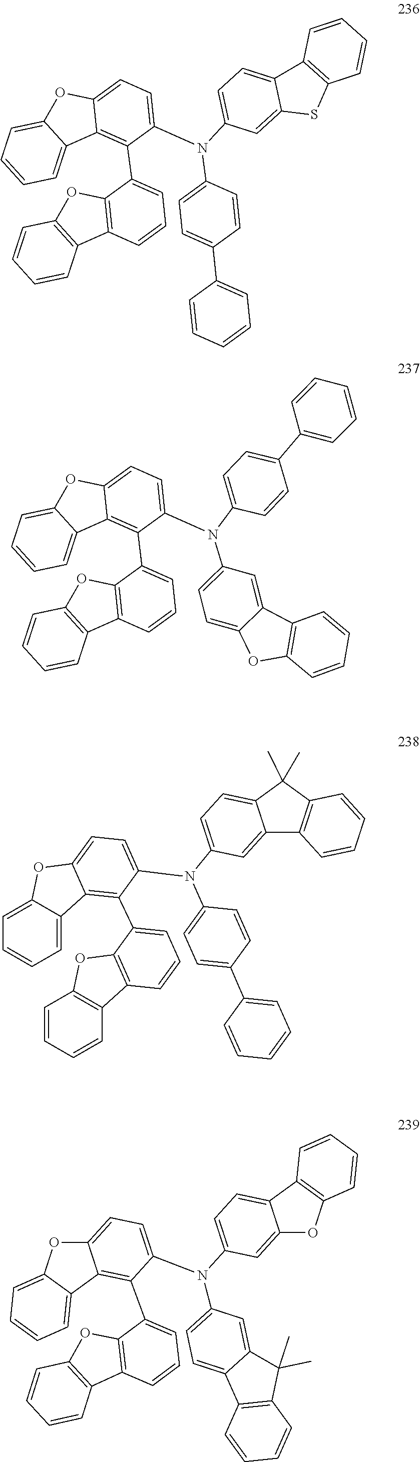 [SMILES: c1ccc(-c2ccc(N(c3ccc4c(c3)sc3ccccc34)c3ccc4oc5ccccc5c4c3-c3cccc4c3oc3ccccc34)cc2)cc1 ; c1ccc(-c2ccc(N(c3ccc4oc5ccccc5c4c3)c3ccc4oc5ccccc5c4c3-c3cccc4c3oc3ccccc34)cc2)cc1 ; CC1(C)c2ccccc2-c2cc(N(c3ccc(-c4ccccc4)cc3)c3ccc4oc5ccccc5c4c3-c3cccc4c3oc3ccccc34)ccc21 ; CC1(C)c2ccccc2-c2ccc(N(c3ccc4c(c3)oc3ccccc34)c3ccc4oc5ccccc5c4c3-c3cccc4c3oc3ccccc34)cc21]

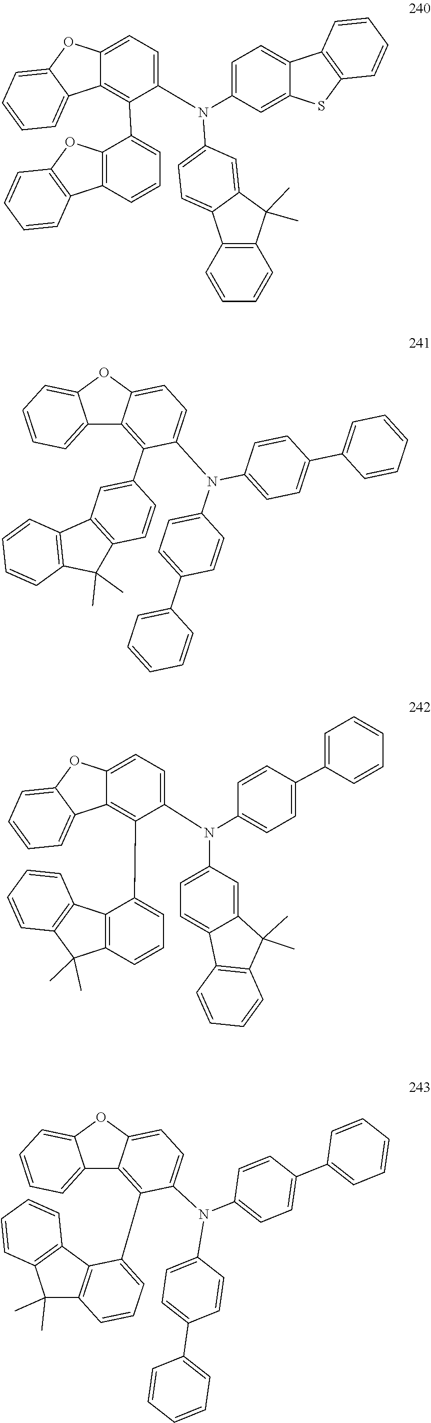 [SMILES: CC1(C)c2ccccc2-c2ccc(N(c3ccc4c(c3)sc3ccccc34)c3ccc4oc5ccccc5c4c3-c3cccc4c3oc3ccccc34)cc21 ; CC1(C)c2ccccc2-c2cc(-c3c(N(c4ccc(-c5ccccc5)cc4)c4ccc(-c5ccccc5)cc4)ccc4oc5ccccc5c34)ccc21 ; CC1(C)c2ccccc2-c2ccc(N(c3ccc(-c4ccccc4)cc3)c3ccc4oc5ccccc5c4c3-c3cccc4c3-c3ccccc3C4(C)C)cc21 ; CC1(C)c2ccccc2-c2c(-c3c(N(c4ccc(-c5ccccc5)cc4)c4ccc(-c5ccccc5)cc4)ccc4oc5ccccc5c34)cccc21]

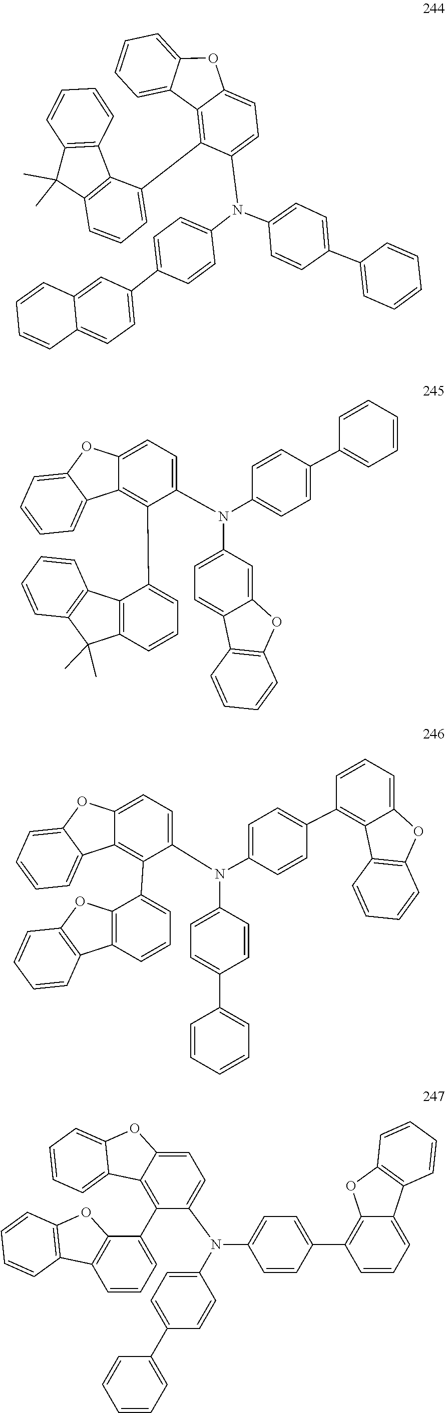 [SMILES: CC1(C)c2ccccc2-c2c(-c3c(N(c4ccc(-c5ccccc5)cc4)c4ccc(-c5ccc6ccccc6c5)cc4)ccc4oc5ccccc5c34)cccc21 ; CC1(C)c2ccccc2-c2c(-c3c(N(c4ccc(-c5ccccc5)cc4)c4ccc5c(c4)oc4ccccc45)ccc4oc5ccccc5c34)cccc21 ; c1ccc(-c2ccc(N(c3ccc(-c4cccc5oc6ccccc6c45)cc3)c3ccc4oc5ccccc5c4c3-c3cccc4c3oc3ccccc34)cc2)cc1 ; c1ccc(-c2ccc(N(c3ccc(-c4cccc5c4oc4ccccc45)cc3)c3ccc4oc5ccccc5c4c3-c3cccc4c3oc3ccccc34)cc2)cc1]

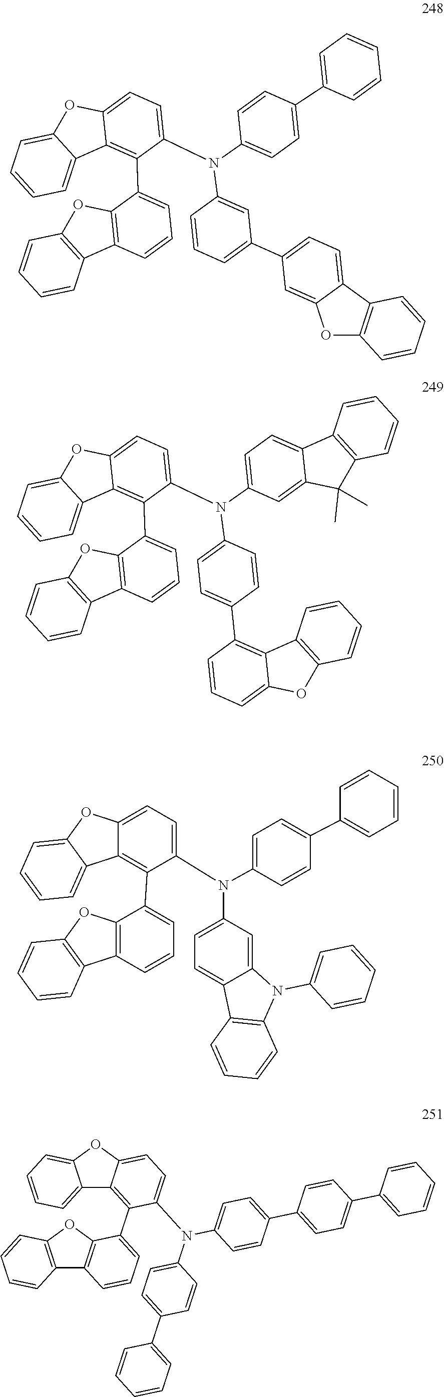

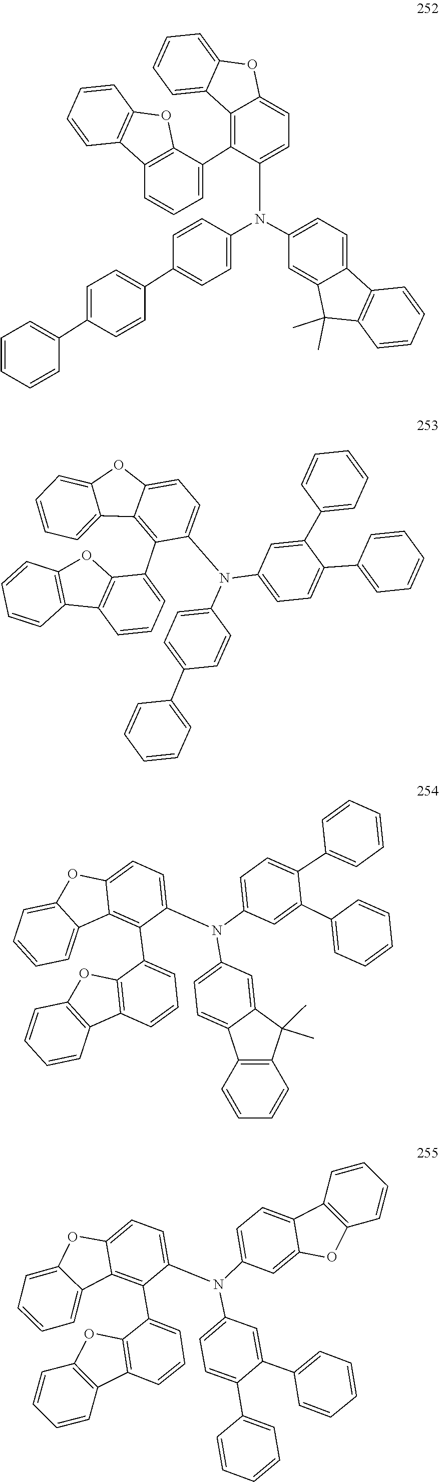 [SMILES: CC1(C)c2ccccc2-c2ccc(N(c3ccc(-c4ccc(-c5ccccc5)cc4)cc3)c3ccc4oc5ccccc5c4c3-c3cccc4c3oc3ccccc34)cc21 ; c1ccc(-c2ccc(N(c3ccc(-c4ccccc4)c(-c4ccccc4)c3)c3ccc4oc5ccccc5c4c3-c3cccc4c3oc3ccccc34)cc2)cc1 ; CC1(C)c2ccccc2-c2ccc(N(c3ccc(-c4ccccc4)c(-c4ccccc4)c3)c3ccc4oc5ccccc5c4c3-c3cccc4c3oc3ccccc34)cc21 ; c1ccc(-c2ccc(N(c3ccc4c(c3)oc3ccccc34)c3ccc4oc5ccccc5c4c3-c3cccc4c3oc3ccccc34)cc2-c2ccccc2)cc1]

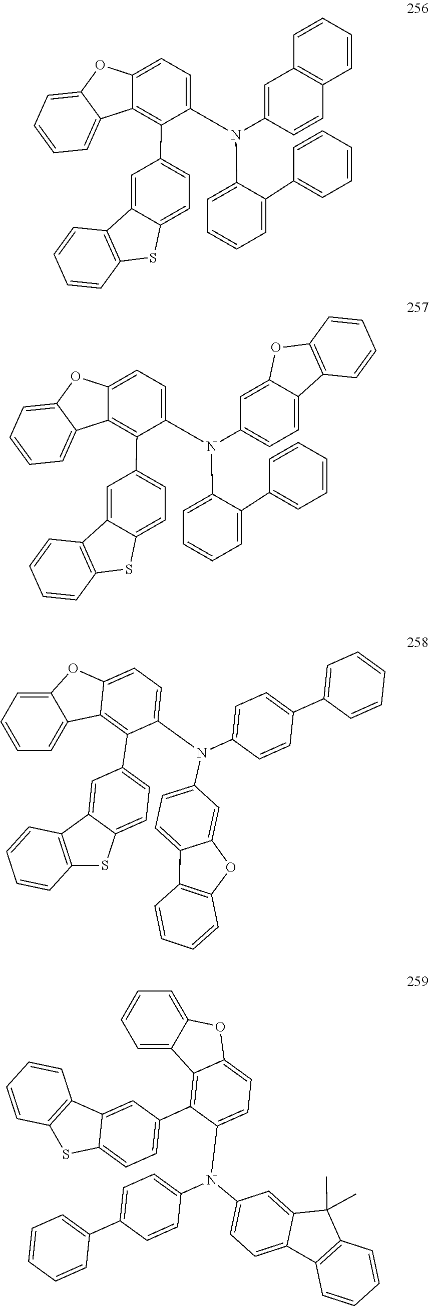 [SMILES: c1ccc(-c2ccccc2N(c2ccc3ccccc3c2)c2ccc3oc4ccccc4c3c2-c2ccc3sc4ccccc4c3c2)cc1 ; c1ccc(-c2ccccc2N(c2ccc3c(c2)oc2ccccc23)c2ccc3oc4ccccc4c3c2-c2ccc3sc4ccccc4c3c2)cc1 ; c1ccc(-c2ccc(N(c3ccc4c(c3)oc3ccccc34)c3ccc4oc5ccccc5c4c3-c3ccc4sc5ccccc5c4c3)cc2)cc1 ; CC1(C)c2ccccc2-c2ccc(N(c3ccc(-c4ccccc4)cc3)c3ccc4oc5ccccc5c4c3-c3ccc4sc5ccccc5c4c3)cc21]

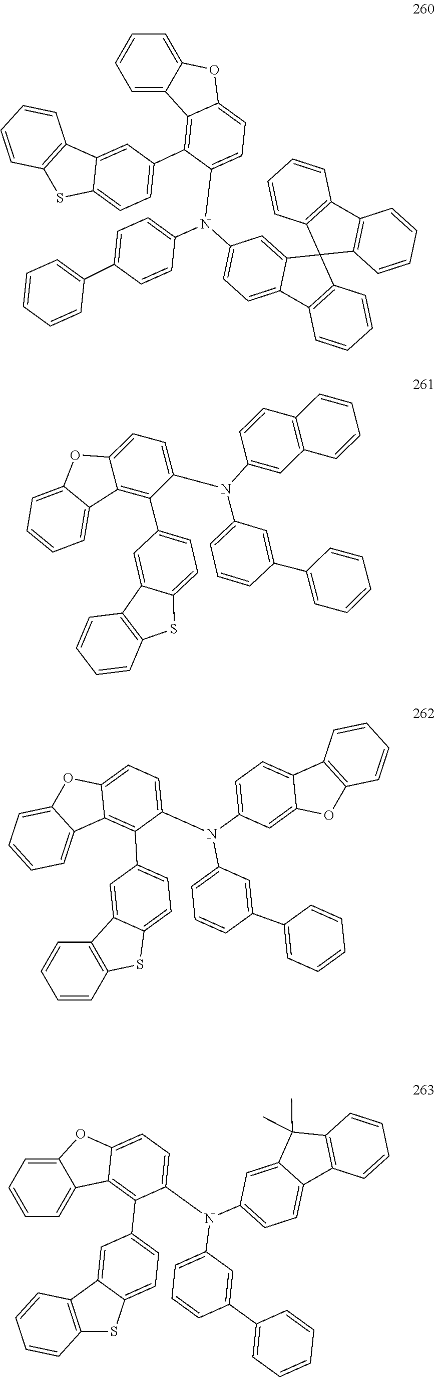 [SMILES: c1ccc(-c2ccc(N(c3ccc4c(c3)C3(c5ccccc5-c5ccccc53)c3ccccc3-4)c3ccc4oc5ccccc5c4c3-c3ccc4sc5ccccc5c4c3)cc2)cc1 ; c1ccc(-c2cccc(N(c3ccc4ccccc4c3)c3ccc4oc5ccccc5c4c3-c3ccc4sc5ccccc5c4c3)c2)cc1 ; c1ccc(-c2cccc(N(c3ccc4c(c3)oc3ccccc34)c3ccc4oc5ccccc5c4c3-c3ccc4sc5ccccc5c4c3)c2)cc1 ; CC1(C)c2ccccc2-c2ccc(N(c3cccc(-c4ccccc4)c3)c3ccc4oc5ccccc5c4c3-c3ccc4sc5ccccc5c4c3)cc21]

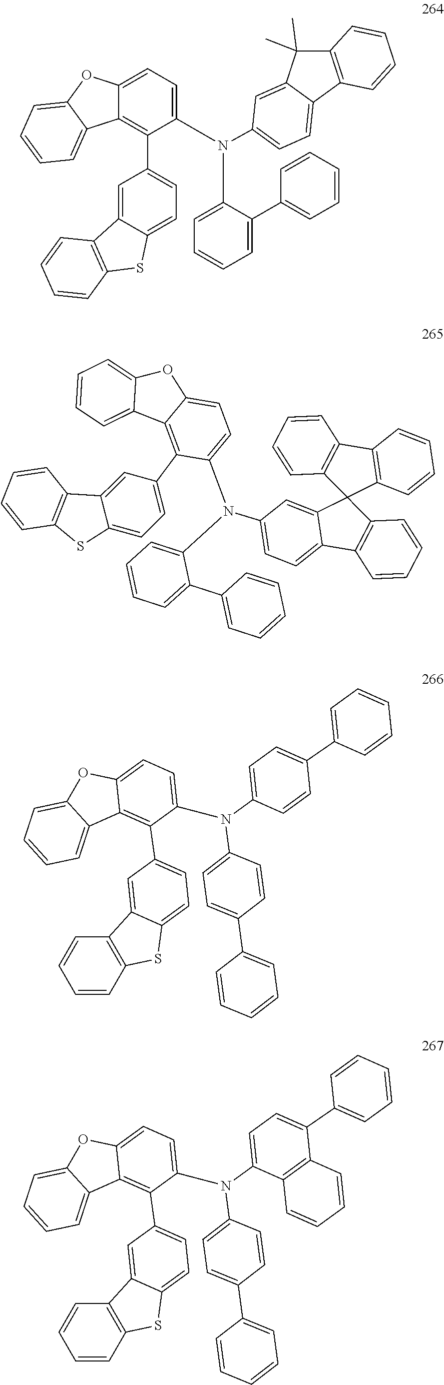 [SMILES: CC1(C)c2ccccc2-c2ccc(N(c3ccccc3-c3ccccc3)c3ccc4oc5ccccc5c4c3-c3ccc4sc5ccccc5c4c3)cc21 ; c1ccc(-c2ccccc2N(c2ccc3c(c2)C2(c4ccccc4-c4ccccc42)c2ccccc2-3)c2ccc3oc4ccccc4c3c2-c2ccc3sc4ccccc4c3c2)cc1 ; c1ccc(-c2ccc(N(c3ccc(-c4ccccc4)cc3)c3ccc4oc5ccccc5c4c3-c3ccc4sc5ccccc5c4c3)cc2)cc1 ; c1ccc(-c2ccc(N(c3ccc4oc5ccccc5c4c3-c3ccc4sc5ccccc5c4c3)c3ccc(-c4ccccc4)c4ccccc34)cc2)cc1]

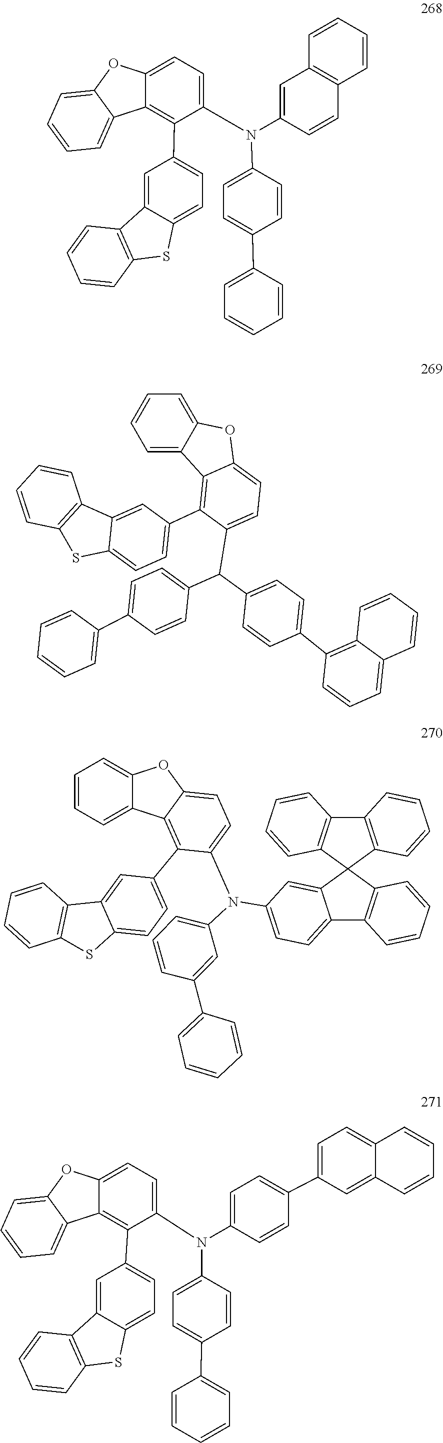 [SMILES: c1ccc(-c2ccc(N(c3ccc4ccccc4c3)c3ccc4oc5ccccc5c4c3-c3ccc4sc5ccccc5c4c3)cc2)cc1 ; c1ccc(-c2ccc(C(c3ccc(-c4cccc5ccccc45)cc3)c3ccc4oc5ccccc5c4c3-c3ccc4sc5ccccc5c4c3)cc2)cc1 ; c1ccc(-c2cccc(N(c3ccc4c(c3)C3(c5ccccc5-c5ccccc53)c3ccccc3-4)c3ccc4oc5ccccc5c4c3-c3ccc4sc5ccccc5c4c3)c2)cc1 ; c1ccc(-c2ccc(N(c3ccc(-c4ccc5ccccc5c4)cc3)c3ccc4oc5ccccc5c4c3-c3ccc4sc5ccccc5c4c3)cc2)cc1]

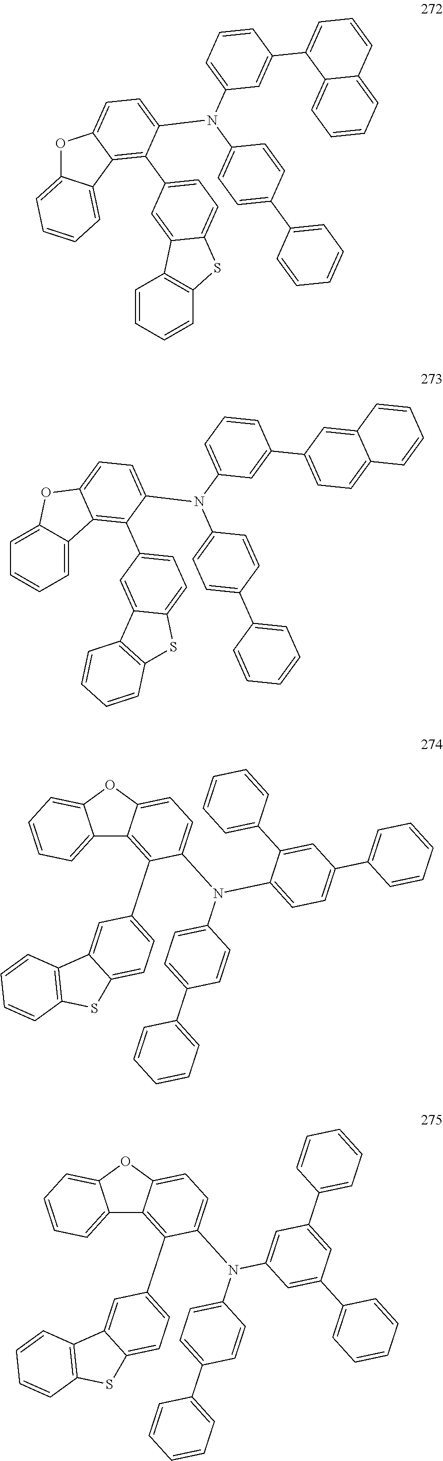 [SMILES: c1ccc(-c2ccc(N(c3cccc(-c4cccc5ccccc45)c3)c3ccc4oc5ccccc5c4c3-c3ccc4sc5ccccc5c4c3)cc2)cc1 ; c1ccc(-c2ccc(N(c3cccc(-c4ccc5ccccc5c4)c3)c3ccc4oc5ccccc5c4c3-c3ccc4sc5ccccc5c4c3)cc2)cc1 ; c1ccc(-c2ccc(N(c3ccc(-c4ccccc4)cc3-c3ccccc3)c3ccc4oc5ccccc5c4c3-c3ccc4sc5ccccc5c4c3)cc2)cc1 ; c1ccc(-c2ccc(N(c3cc(-c4ccccc4)cc(-c4ccccc4)c3)c3ccc4oc5ccccc5c4c3-c3ccc4sc5ccccc5c4c3)cc2)cc1]

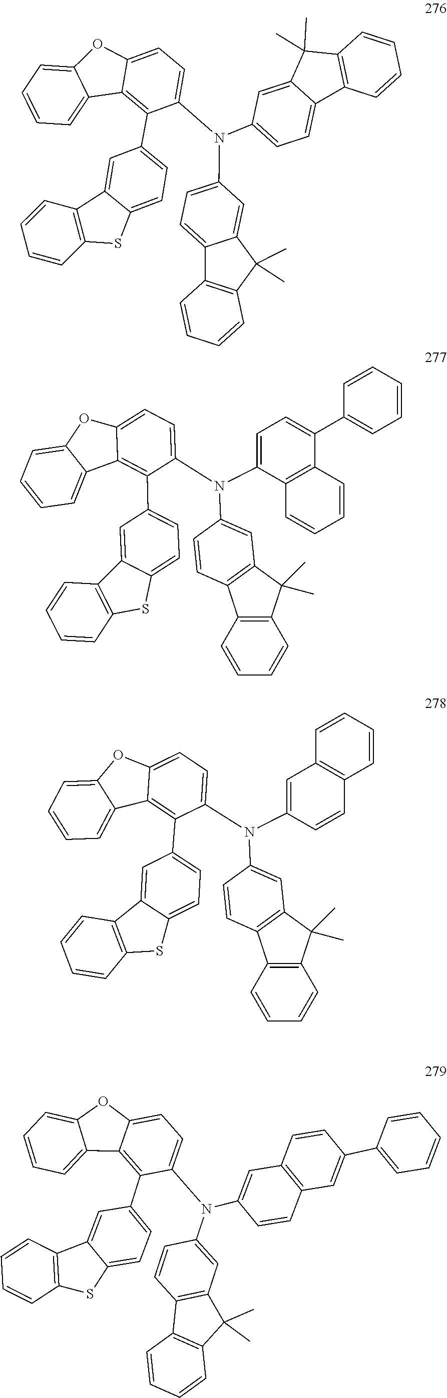 [SMILES: CC1(C)c2ccccc2-c2ccc(N(c3ccc4c(c3)C(C)(C)c3ccccc3-4)c3ccc4oc5ccccc5c4c3-c3ccc4sc5ccccc5c4c3)cc21 ; CC1(C)c2ccccc2-c2ccc(N(c3ccc4oc5ccccc5c4c3-c3ccc4sc5ccccc5c4c3)c3ccc(-c4ccccc4)c4ccccc34)cc21 ; CC1(C)c2ccccc2-c2ccc(N(c3ccc4ccccc4c3)c3ccc4oc5ccccc5c4c3-c3ccc4sc5ccccc5c4c3)cc21 ; CC1(C)c2ccccc2-c2ccc(N(c3ccc4cc(-c5ccccc5)ccc4c3)c3ccc4oc5ccccc5c4c3-c3ccc4sc5ccccc5c4c3)cc21]

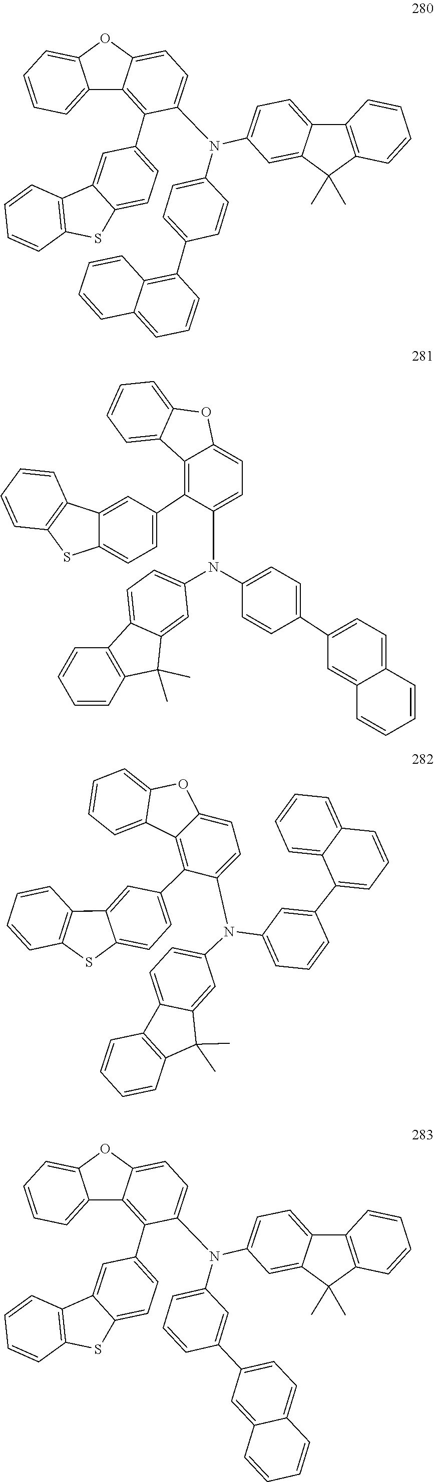

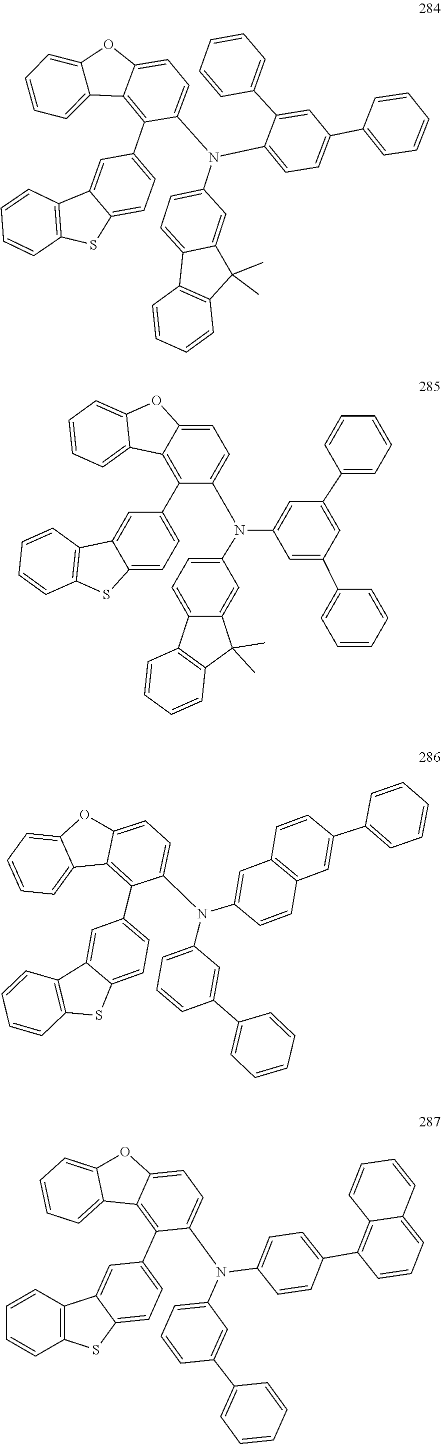 [SMILES: CC1(C)c2ccccc2-c2ccc(N(c3ccc(-c4ccccc4)cc3-c3ccccc3)c3ccc4oc5ccccc5c4c3-c3ccc4sc5ccccc5c4c3)cc21 ; CC1(C)c2ccccc2-c2ccc(N(c3cc(-c4ccccc4)cc(-c4ccccc4)c3)c3ccc4oc5ccccc5c4c3-c3ccc4sc5ccccc5c4c3)cc21 ; c1ccc(-c2cccc(N(c3ccc4cc(-c5ccccc5)ccc4c3)c3ccc4oc5ccccc5c4c3-c3ccc4sc5ccccc5c4c3)c2)cc1 ; c1ccc(-c2cccc(N(c3ccc(-c4cccc5ccccc45)cc3)c3ccc4oc5ccccc5c4c3-c3ccc4sc5ccccc5c4c3)c2)cc1]

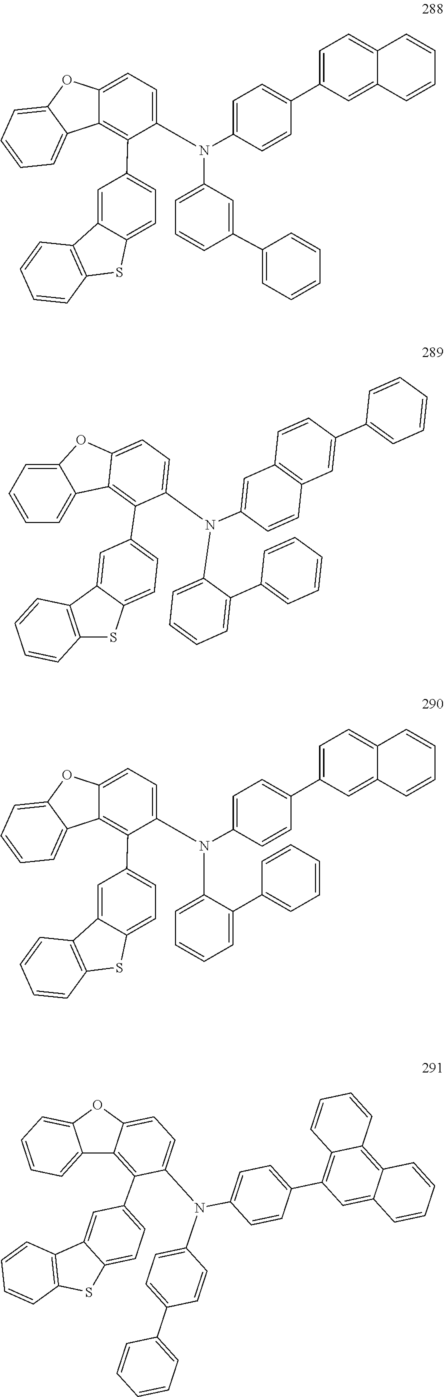 [SMILES: c1ccc(-c2cccc(N(c3ccc(-c4ccc5ccccc5c4)cc3)c3ccc4oc5ccccc5c4c3-c3ccc4sc5ccccc5c4c3)c2)cc1 ; c1ccc(-c2ccc3cc(N(c4ccccc4-c4ccccc4)c4ccc5oc6ccccc6c5c4-c4ccc5sc6ccccc6c5c4)ccc3c2)cc1 ; c1ccc(-c2ccccc2N(c2ccc(-c3ccc4ccccc4c3)cc2)c2ccc3oc4ccccc4c3c2-c2ccc3sc4ccccc4c3c2)cc1 ; c1ccc(-c2ccc(N(c3ccc(-c4cc5ccccc5c5ccccc45)cc3)c3ccc4oc5ccccc5c4c3-c3ccc4sc5ccccc5c4c3)cc2)cc1]

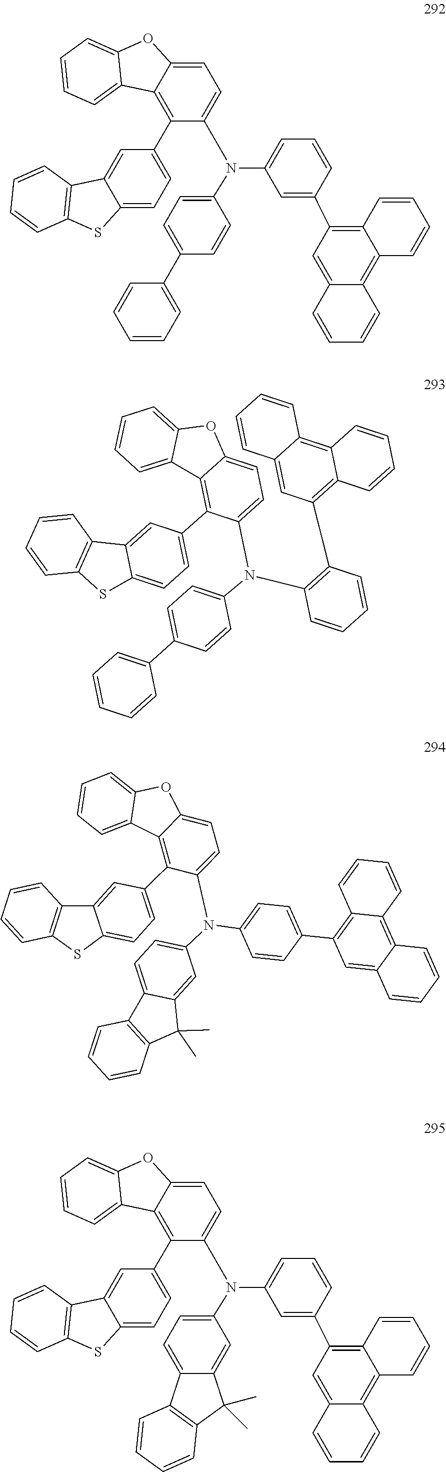 [SMILES: c1ccc(-c2ccc(N(c3cccc(-c4cc5ccccc5c5ccccc45)c3)c3ccc4oc5ccccc5c4c3-c3ccc4sc5ccccc5c4c3)cc2)cc1 ; c1ccc(-c2ccc(N(c3ccccc3-c3cc4ccccc4c4ccccc34)c3ccc4oc5ccccc5c4c3-c3ccc4sc5ccccc5c4c3)cc2)cc1 ; CC1(C)c2ccccc2-c2ccc(N(c3ccc(-c4cc5ccccc5c5ccccc45)cc3)c3ccc4oc5ccccc5c4c3-c3ccc4sc5ccccc5c4c3)cc21 ; CC1(C)c2ccccc2-c2ccc(N(c3cccc(-c4cc5ccccc5c5ccccc45)c3)c3ccc4oc5ccccc5c4c3-c3ccc4sc5ccccc5c4c3)cc21]

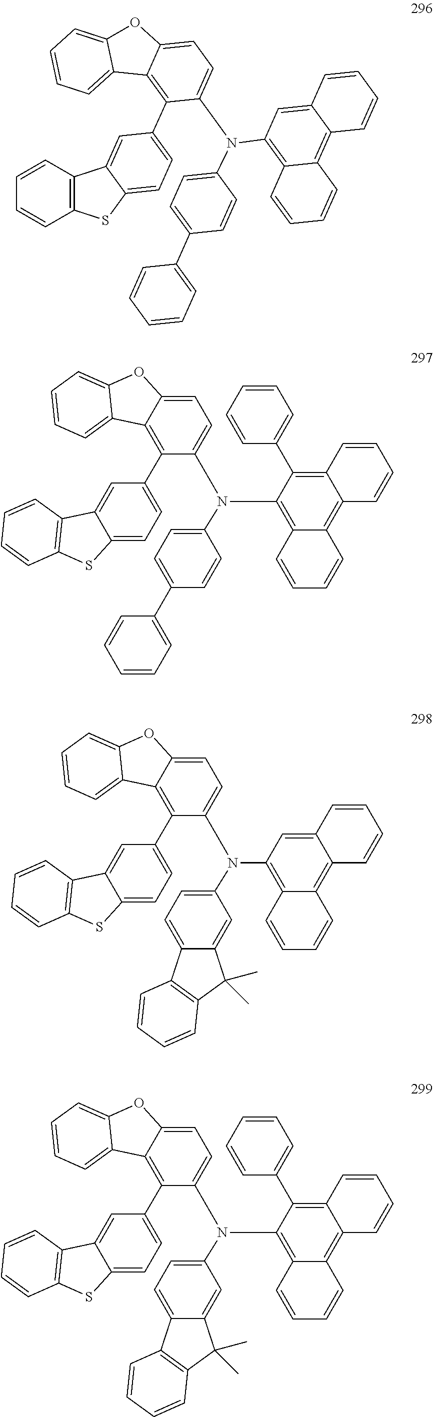 [SMILES: c1ccc(-c2ccc(N(c3ccc4oc5ccccc5c4c3-c3ccc4sc5ccccc5c4c3)c3cc4ccccc4c4ccccc34)cc2)cc1 ; c1ccc(-c2ccc(N(c3ccc4oc5ccccc5c4c3-c3ccc4sc5ccccc5c4c3)c3c(-c4ccccc4)c4ccccc4c4ccccc34)cc2)cc1 ; CC1(C)c2ccccc2-c2ccc(N(c3ccc4oc5ccccc5c4c3-c3ccc4sc5ccccc5c4c3)c3cc4ccccc4c4ccccc34)cc21 ; CC1(C)c2ccccc2-c2ccc(N(c3ccc4oc5ccccc5c4c3-c3ccc4sc5ccccc5c4c3)c3c(-c4ccccc4)c4ccccc4c4ccccc34)cc21]

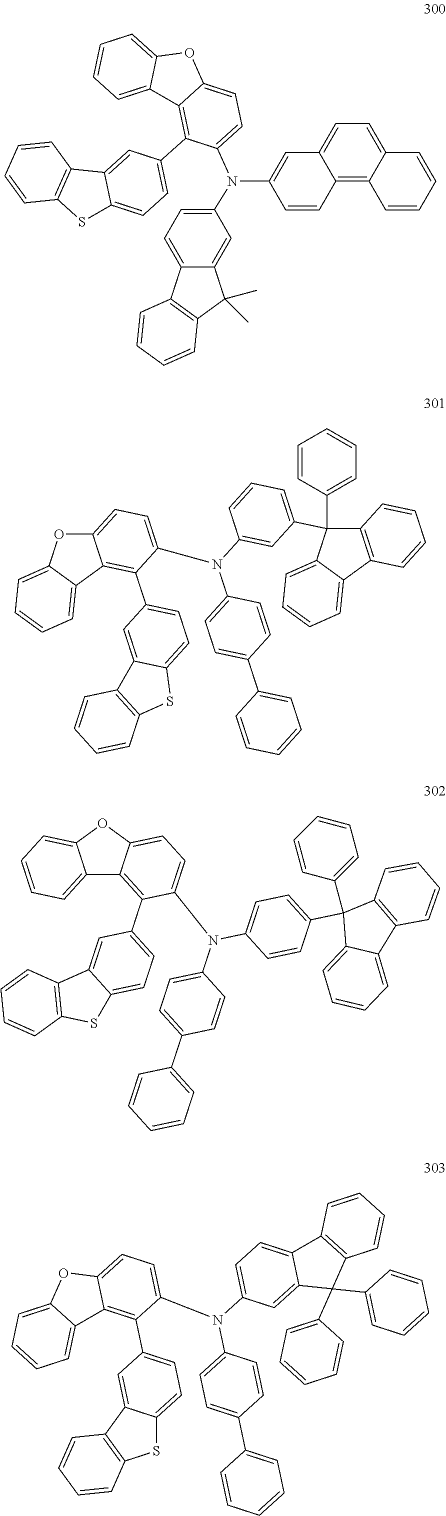 [SMILES: CC1(C)c2ccccc2-c2ccc(N(c3ccc4c(ccc5ccccc54)c3)c3ccc4oc5ccccc5c4c3-c3ccc4sc5ccccc5c4c3)cc21 ; c1ccc(-c2ccc(N(c3cccc(C4(c5ccccc5)c5ccccc5-c5ccccc54)c3)c3ccc4oc5ccccc5c4c3-c3ccc4sc5ccccc5c4c3)cc2)cc1 ; c1ccc(-c2ccc(N(c3ccc(C4(c5ccccc5)c5ccccc5-c5ccccc54)cc3)c3ccc4oc5ccccc5c4c3-c3ccc4sc5ccccc5c4c3)cc2)cc1 ; c1ccc(-c2ccc(N(c3ccc4c(c3)C(c3ccccc3)(c3ccccc3)c3ccccc3-4)c3ccc4oc5ccccc5c4c3-c3ccc4sc5ccccc5c4c3)cc2)cc1]

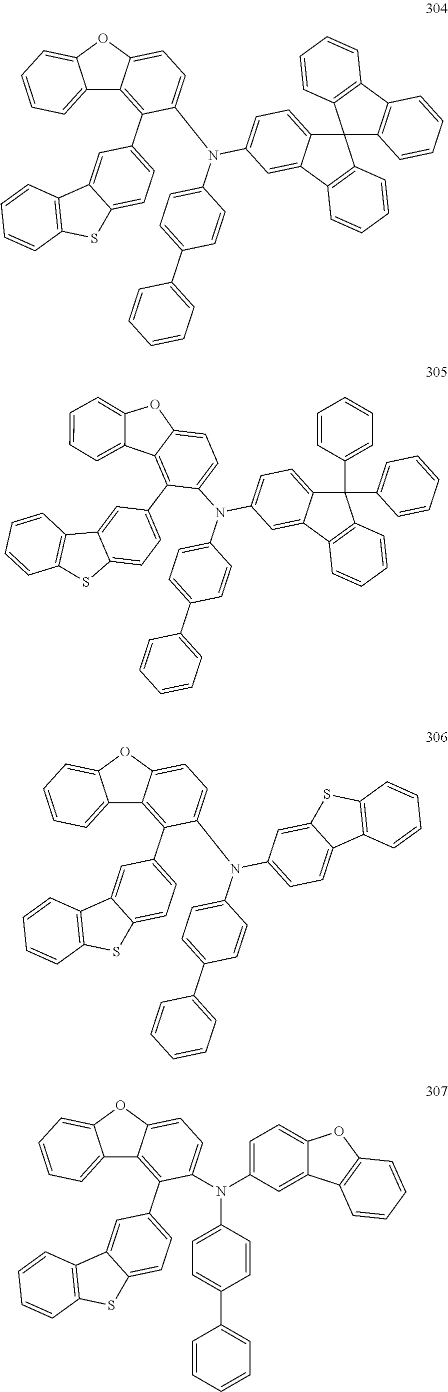 [SMILES: c1ccc(-c2ccc(N(c3ccc4c(c3)-c3ccccc3C43c4ccccc4-c4ccccc43)c3ccc4oc5ccccc5c4c3-c3ccc4sc5ccccc5c4c3)cc2)cc1 ; c1ccc(-c2ccc(N(c3ccc4c(c3)-c3ccccc3C4(c3ccccc3)c3ccccc3)c3ccc4oc5ccccc5c4c3-c3ccc4sc5ccccc5c4c3)cc2)cc1 ; c1ccc(-c2ccc(N(c3ccc4c(c3)sc3ccccc34)c3ccc4oc5ccccc5c4c3-c3ccc4sc5ccccc5c4c3)cc2)cc1 ; c1ccc(-c2ccc(N(c3ccc4oc5ccccc5c4c3)c3ccc4oc5ccccc5c4c3-c3ccc4sc5ccccc5c4c3)cc2)cc1]

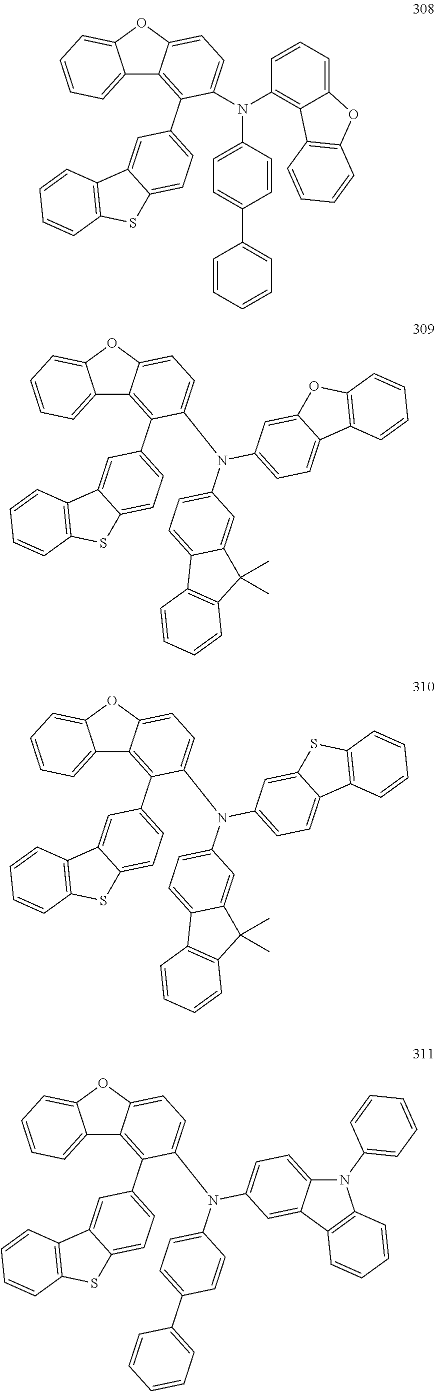 [SMILES: c1ccc(-c2ccc(N(c3ccc4oc5ccccc5c4c3-c3ccc4sc5ccccc5c4c3)c3cccc4oc5ccccc5c34)cc2)cc1 ; CC1(C)c2ccccc2-c2ccc(N(c3ccc4c(c3)oc3ccccc34)c3ccc4oc5ccccc5c4c3-c3ccc4sc5ccccc5c4c3)cc21 ; CC1(C)c2ccccc2-c2ccc(N(c3ccc4c(c3)sc3ccccc34)c3ccc4oc5ccccc5c4c3-c3ccc4sc5ccccc5c4c3)cc21 ; c1ccc(-c2ccc(N(c3ccc4c(c3)c3ccccc3n4-c3ccccc3)c3ccc4oc5ccccc5c4c3-c3ccc4sc5ccccc5c4c3)cc2)cc1]

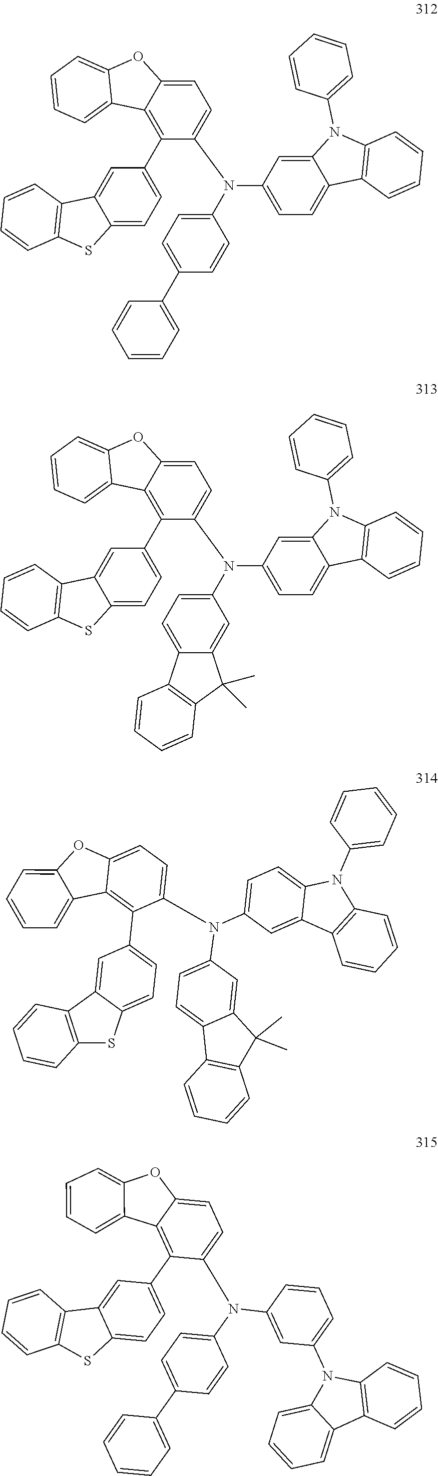 [SMILES: c1ccc(-c2ccc(N(c3ccc4c5ccccc5n(-c5ccccc5)c4c3)c3ccc4oc5ccccc5c4c3-c3ccc4sc5ccccc5c4c3)cc2)cc1 ; CC1(C)c2ccccc2-c2ccc(N(c3ccc4c5ccccc5n(-c5ccccc5)c4c3)c3ccc4oc5ccccc5c4c3-c3ccc4sc5ccccc5c4c3)cc21 ; CC1(C)c2ccccc2-c2ccc(N(c3ccc4c(c3)c3ccccc3n4-c3ccccc3)c3ccc4oc5ccccc5c4c3-c3ccc4sc5ccccc5c4c3)cc21 ; c1ccc(-c2ccc(N(c3cccc(-n4c5ccccc5c5ccccc54)c3)c3ccc4oc5ccccc5c4c3-c3ccc4sc5ccccc5c4c3)cc2)cc1]

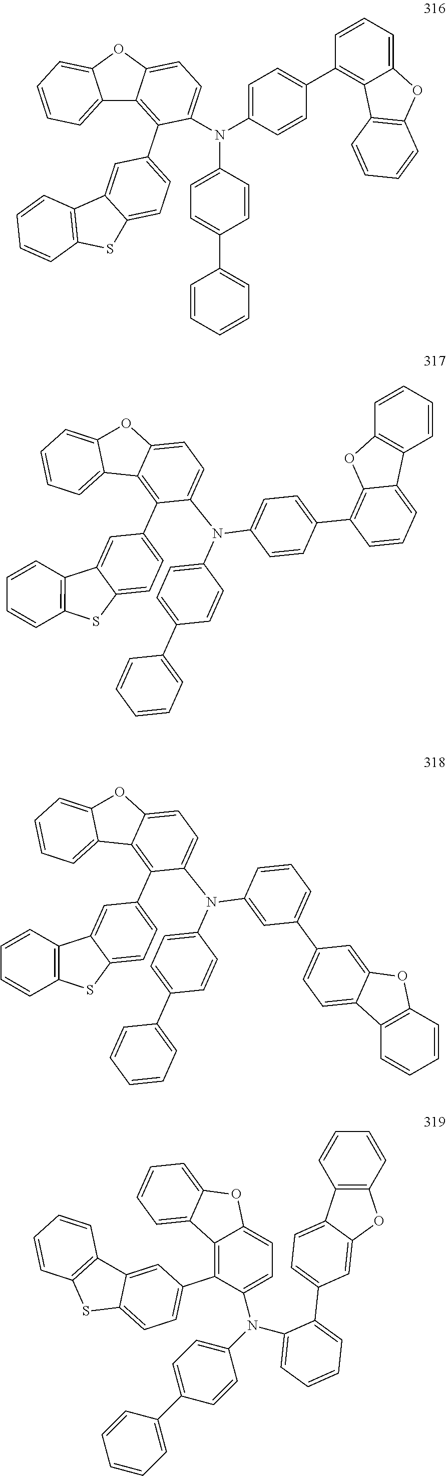 [SMILES: c1ccc(-c2ccc(N(c3ccc(-c4cccc5oc6ccccc6c45)cc3)c3ccc4oc5ccccc5c4c3-c3ccc4sc5ccccc5c4c3)cc2)cc1 ; c1ccc(-c2ccc(N(c3ccc(-c4cccc5c4oc4ccccc45)cc3)c3ccc4oc5ccccc5c4c3-c3ccc4sc5ccccc5c4c3)cc2)cc1 ; c1ccc(-c2ccc(N(c3cccc(-c4ccc5c(c4)oc4ccccc45)c3)c3ccc4oc5ccccc5c4c3-c3ccc4sc5ccccc5c4c3)cc2)cc1 ; c1ccc(-c2ccc(N(c3ccccc3-c3ccc4c(c3)oc3ccccc34)c3ccc4oc5ccccc5c4c3-c3ccc4sc5ccccc5c4c3)cc2)cc1]

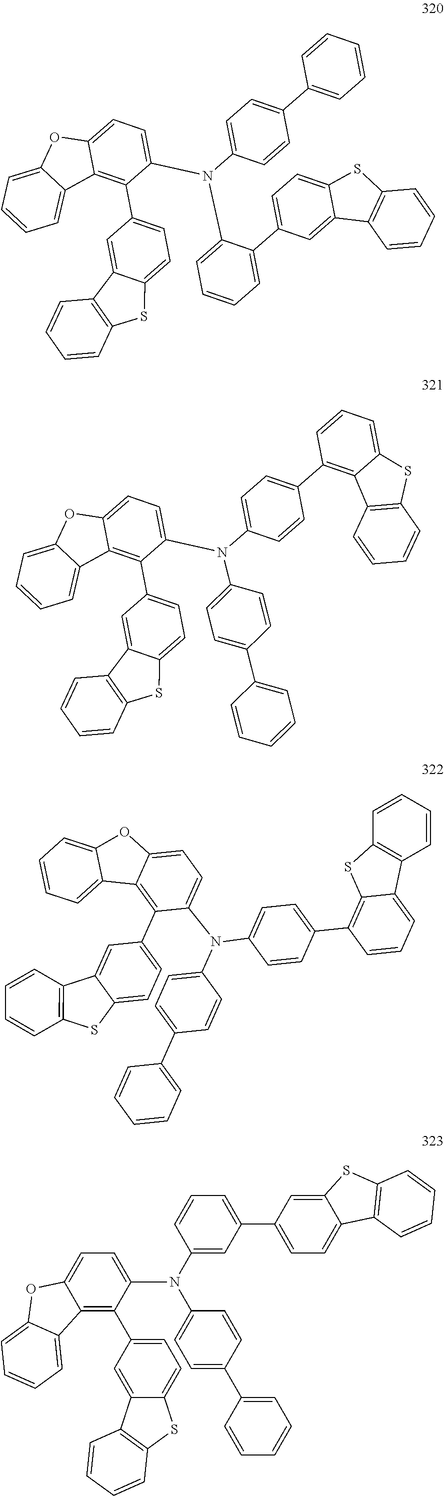 [SMILES: c1ccc(-c2ccc(N(c3ccccc3-c3ccc4sc5ccccc5c4c3)c3ccc4oc5ccccc5c4c3-c3ccc4sc5ccccc5c4c3)cc2)cc1 ; c1ccc(-c2ccc(N(c3ccc(-c4cccc5sc6ccccc6c45)cc3)c3ccc4oc5ccccc5c4c3-c3ccc4sc5ccccc5c4c3)cc2)cc1 ; c1ccc(-c2ccc(N(c3ccc(-c4cccc5c4sc4ccccc45)cc3)c3ccc4oc5ccccc5c4c3-c3ccc4sc5ccccc5c4c3)cc2)cc1 ; c1ccc(-c2ccc(N(c3cccc(-c4ccc5c(c4)sc4ccccc45)c3)c3ccc4oc5ccccc5c4c3-c3ccc4sc5ccccc5c4c3)cc2)cc1]

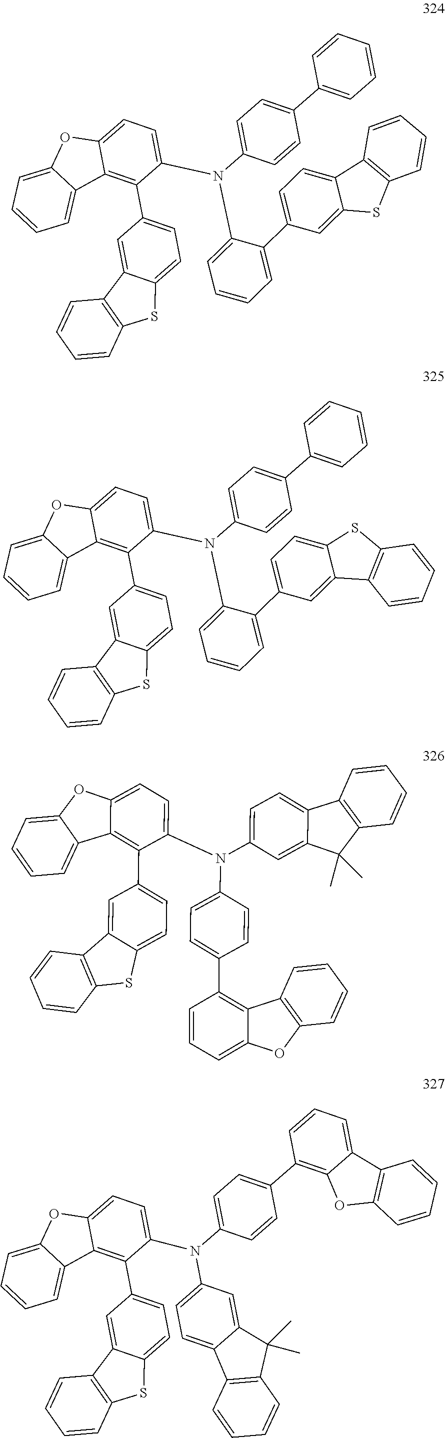 [SMILES: c1ccc(-c2ccc(N(c3ccccc3-c3ccc4c(c3)sc3ccccc34)c3ccc4oc5ccccc5c4c3-c3ccc4sc5ccccc5c4c3)cc2)cc1 ; c1ccc(-c2ccc(N(c3ccccc3-c3ccc4sc5ccccc5c4c3)c3ccc4oc5ccccc5c4c3-c3ccc4sc5ccccc5c4c3)cc2)cc1 ; CC1(C)c2ccccc2-c2ccc(N(c3ccc(-c4cccc5oc6ccccc6c45)cc3)c3ccc4oc5ccccc5c4c3-c3ccc4sc5ccccc5c4c3)cc21 ; CC1(C)c2ccccc2-c2ccc(N(c3ccc(-c4cccc5c4oc4ccccc45)cc3)c3ccc4oc5ccccc5c4c3-c3ccc4sc5ccccc5c4c3)cc21]

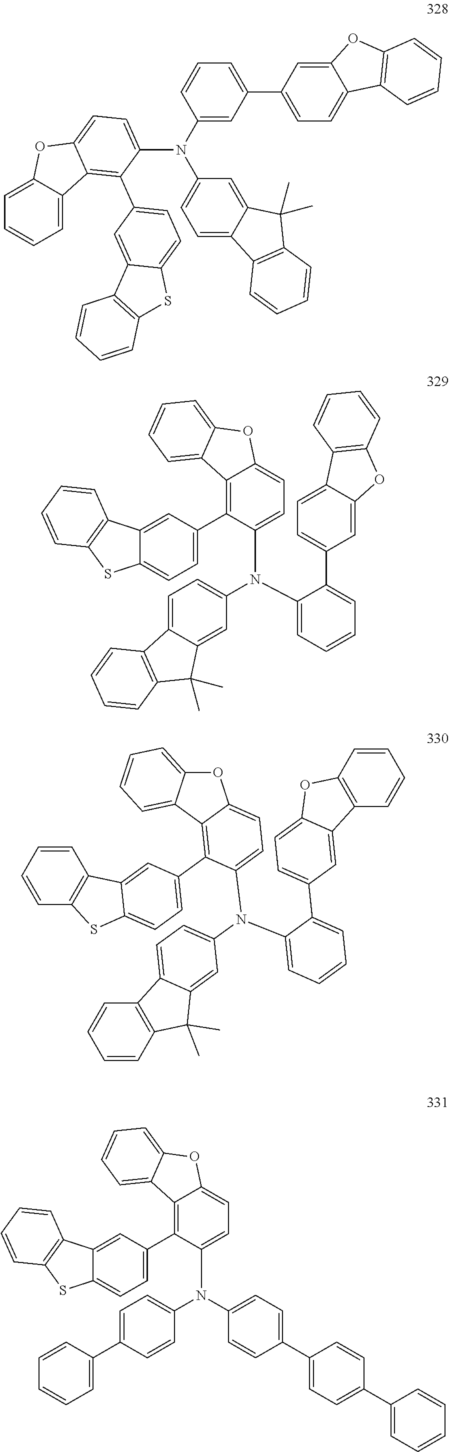

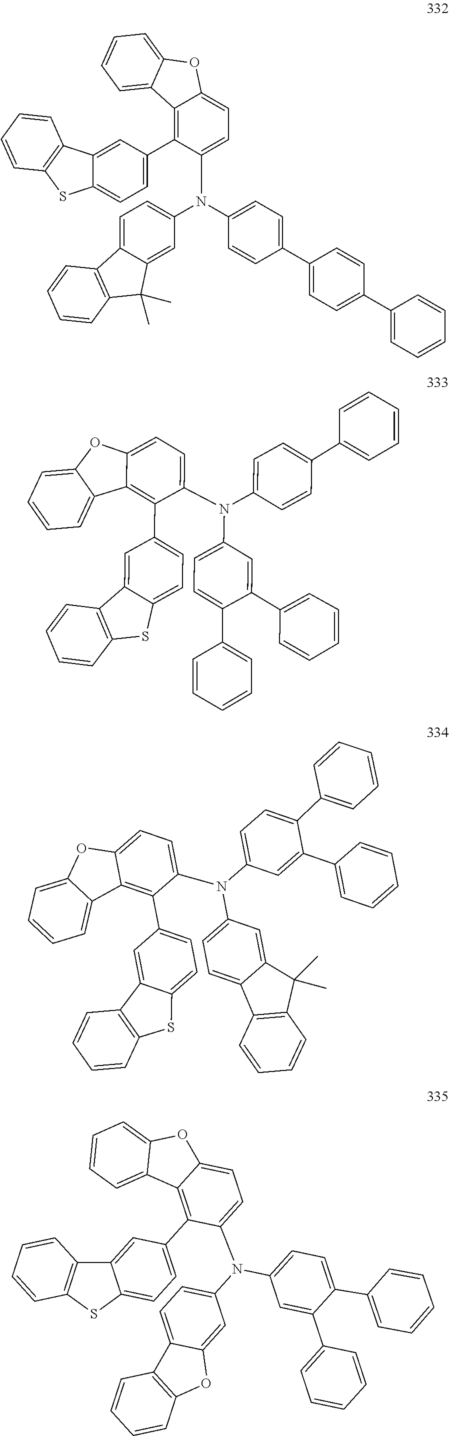 [SMILES: CC1(C)c2ccccc2-c2ccc(N(c3ccc(-c4ccc(-c5ccccc5)cc4)cc3)c3ccc4oc5ccccc5c4c3-c3ccc4sc5ccccc5c4c3)cc21 ; c1ccc(-c2ccc(N(c3ccc(-c4ccccc4)c(-c4ccccc4)c3)c3ccc4oc5ccccc5c4c3-c3ccc4sc5ccccc5c4c3)cc2)cc1 ; CC1(C)c2ccccc2-c2ccc(N(c3ccc(-c4ccccc4)c(-c4ccccc4)c3)c3ccc4oc5ccccc5c4c3-c3ccc4sc5ccccc5c4c3)cc21 ; c1ccc(-c2ccc(N(c3ccc4c(c3)oc3ccccc34)c3ccc4oc5ccccc5c4c3-c3ccc4sc5ccccc5c4c3)cc2-c2ccccc2)cc1]

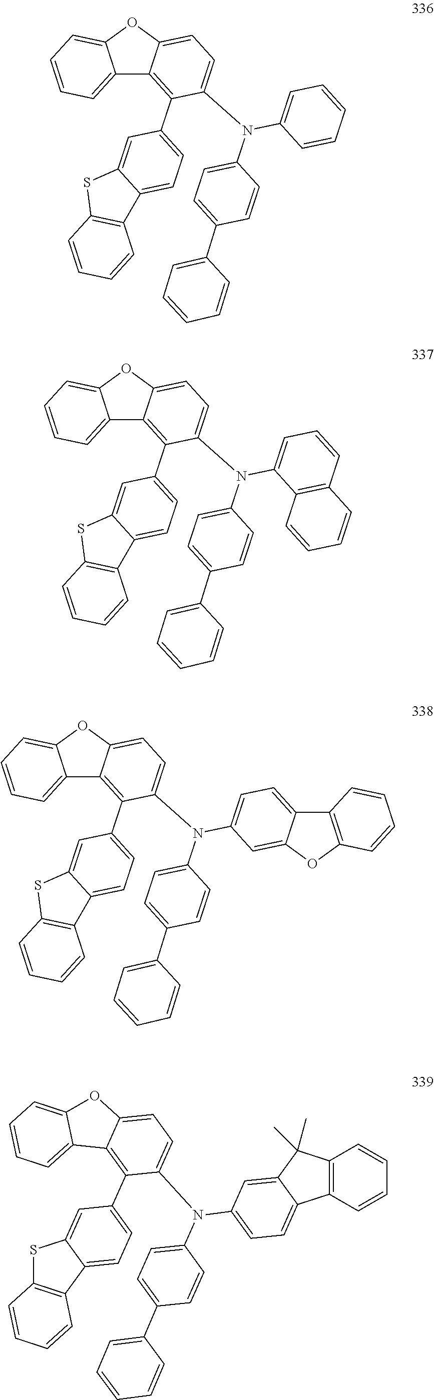 [SMILES: c1ccc(-c2ccc(N(c3ccccc3)c3ccc4oc5ccccc5c4c3-c3ccc4c(c3)sc3ccccc34)cc2)cc1 ; c1ccc(-c2ccc(N(c3ccc4oc5ccccc5c4c3-c3ccc4c(c3)sc3ccccc34)c3cccc4ccccc34)cc2)cc1 ; c1ccc(-c2ccc(N(c3ccc4c(c3)oc3ccccc34)c3ccc4oc5ccccc5c4c3-c3ccc4c(c3)sc3ccccc34)cc2)cc1 ; CC1(C)c2ccccc2-c2ccc(N(c3ccc(-c4ccccc4)cc3)c3ccc4oc5ccccc5c4c3-c3ccc4c(c3)sc3ccccc34)cc21]

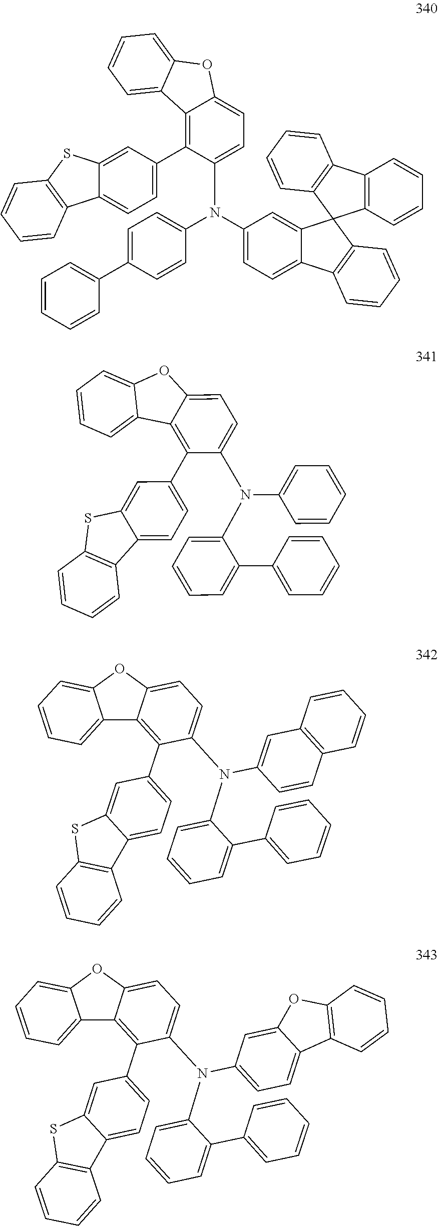

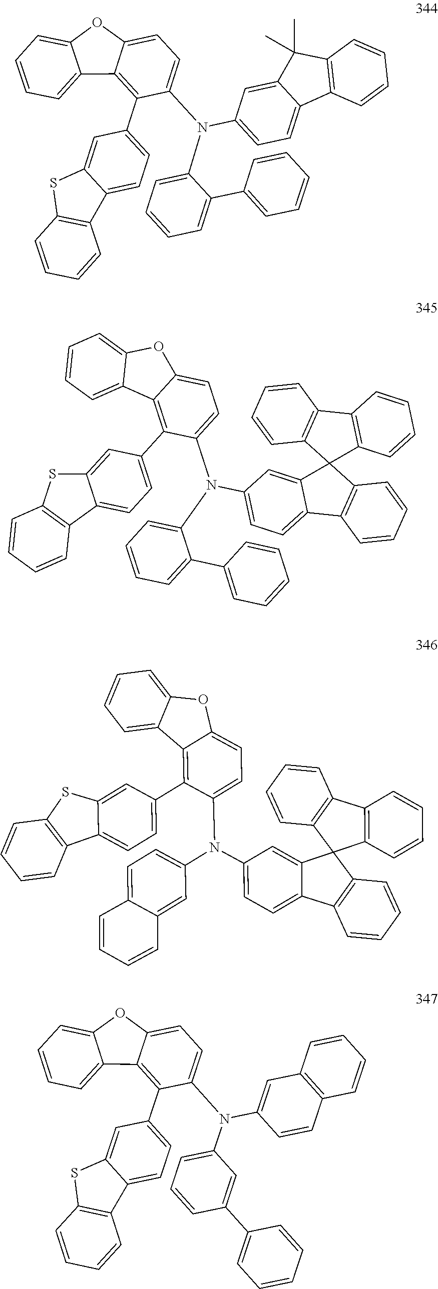 [SMILES: CC1(C)c2ccccc2-c2ccc(N(c3ccccc3-c3ccccc3)c3ccc4oc5ccccc5c4c3-c3ccc4c(c3)sc3ccccc34)cc21 ; c1ccc(-c2ccccc2N(c2ccc3c(c2)C2(c4ccccc4-c4ccccc42)c2ccccc2-3)c2ccc3oc4ccccc4c3c2-c2ccc3c(c2)sc2ccccc23)cc1 ; c1ccc2c(c1)-c1ccccc1C21c2ccccc2-c2ccc(N(c3ccc4ccccc4c3)c3ccc4oc5ccccc5c4c3-c3ccc4c(c3)sc3ccccc34)cc21 ; c1ccc(-c2cccc(N(c3ccc4ccccc4c3)c3ccc4oc5ccccc5c4c3-c3ccc4c(c3)sc3ccccc34)c2)cc1]

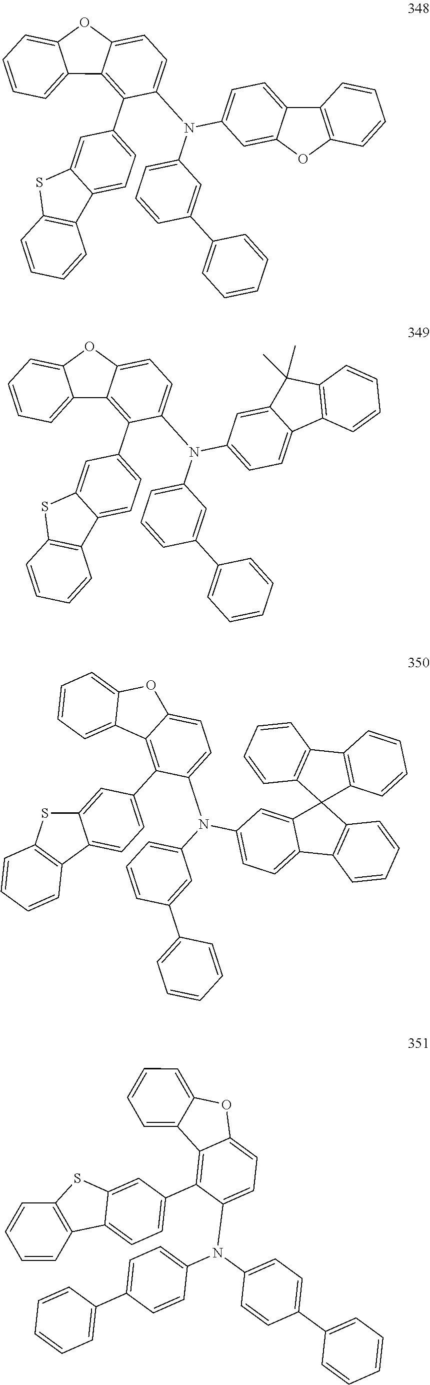 [SMILES: c1ccc(-c2cccc(N(c3ccc4c(c3)oc3ccccc34)c3ccc4oc5ccccc5c4c3-c3ccc4c(c3)sc3ccccc34)c2)cc1 ; CC1(C)c2ccccc2-c2ccc(N(c3cccc(-c4ccccc4)c3)c3ccc4oc5ccccc5c4c3-c3ccc4c(c3)sc3ccccc34)cc21 ; c1ccc(-c2cccc(N(c3ccc4c(c3)C3(c5ccccc5-c5ccccc53)c3ccccc3-4)c3ccc4oc5ccccc5c4c3-c3ccc4c(c3)sc3ccccc34)c2)cc1 ; c1ccc(-c2ccc(N(c3ccc(-c4ccccc4)cc3)c3ccc4oc5ccccc5c4c3-c3ccc4c(c3)sc3ccccc34)cc2)cc1]

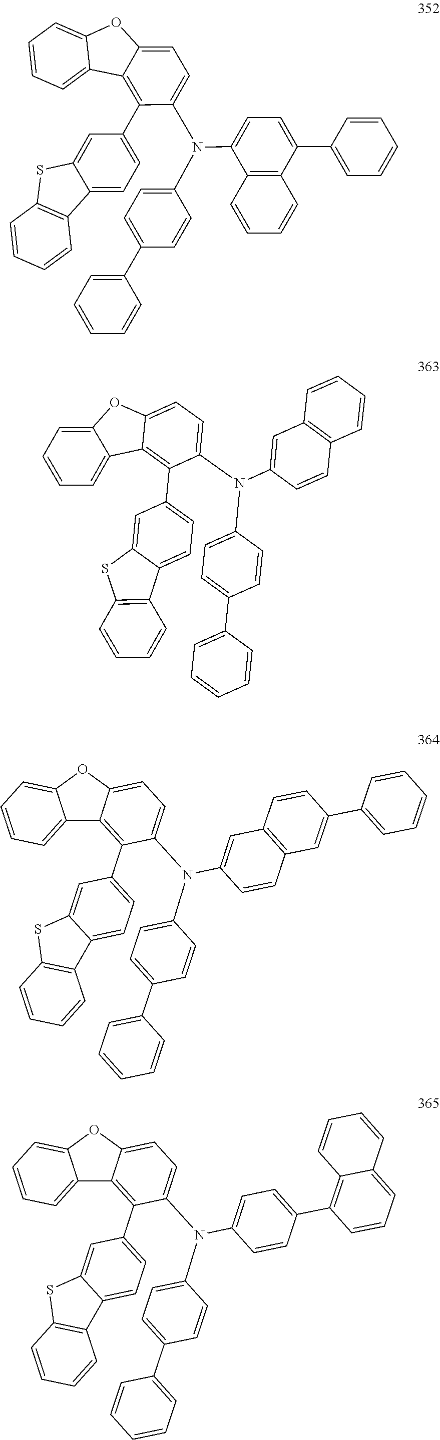 [SMILES: c1ccc(-c2ccc(N(c3ccc4oc5ccccc5c4c3-c3ccc4c(c3)sc3ccccc34)c3ccc(-c4ccccc4)c4ccccc34)cc2)cc1 ; c1ccc(-c2ccc(N(c3ccc4ccccc4c3)c3ccc4oc5ccccc5c4c3-c3ccc4c(c3)sc3ccccc34)cc2)cc1 ; c1ccc(-c2ccc(N(c3ccc4cc(-c5ccccc5)ccc4c3)c3ccc4oc5ccccc5c4c3-c3ccc4c(c3)sc3ccccc34)cc2)cc1 ; c1ccc(-c2ccc(N(c3ccc(-c4cccc5ccccc45)cc3)c3ccc4oc5ccccc5c4c3-c3ccc4c(c3)sc3ccccc34)cc2)cc1]

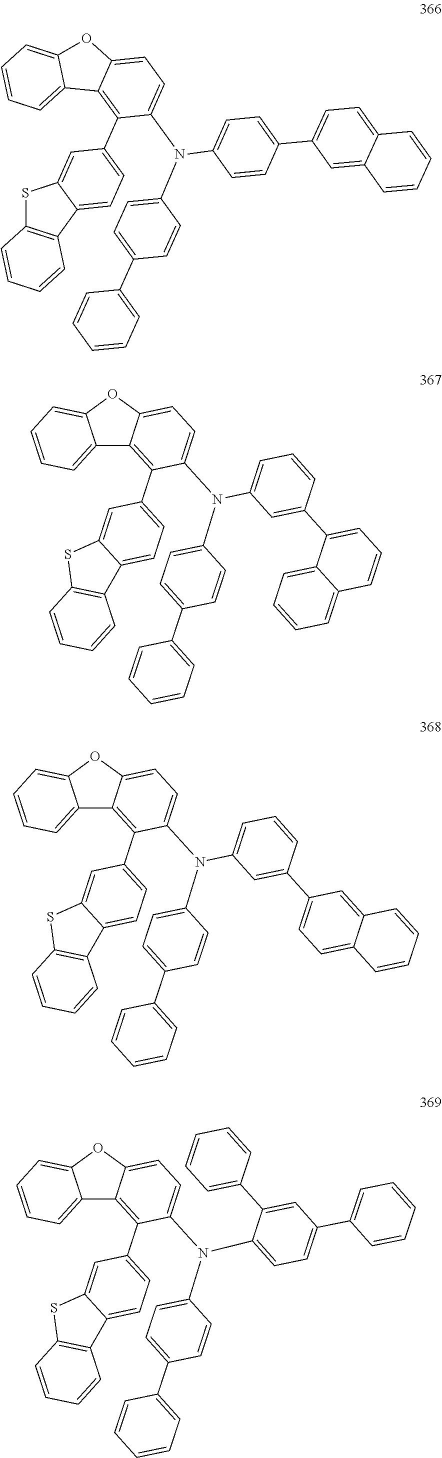 [SMILES: c1ccc(-c2ccc(N(c3ccc(-c4ccc5ccccc5c4)cc3)c3ccc4oc5ccccc5c4c3-c3ccc4c(c3)sc3ccccc34)cc2)cc1 ; c1ccc(-c2ccc(N(c3cccc(-c4cccc5ccccc45)c3)c3ccc4oc5ccccc5c4c3-c3ccc4c(c3)sc3ccccc34)cc2)cc1 ; c1ccc(-c2ccc(N(c3cccc(-c4ccc5ccccc5c4)c3)c3ccc4oc5ccccc5c4c3-c3ccc4c(c3)sc3ccccc34)cc2)cc1 ; c1ccc(-c2ccc(N(c3ccc(-c4ccccc4)cc3-c3ccccc3)c3ccc4oc5ccccc5c4c3-c3ccc4c(c3)sc3ccccc34)cc2)cc1]

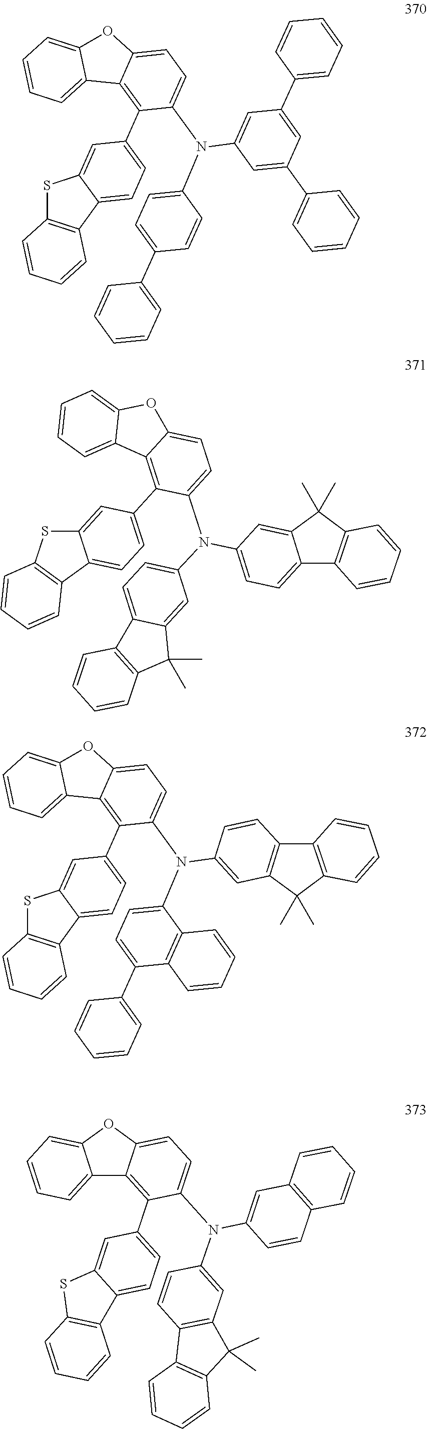

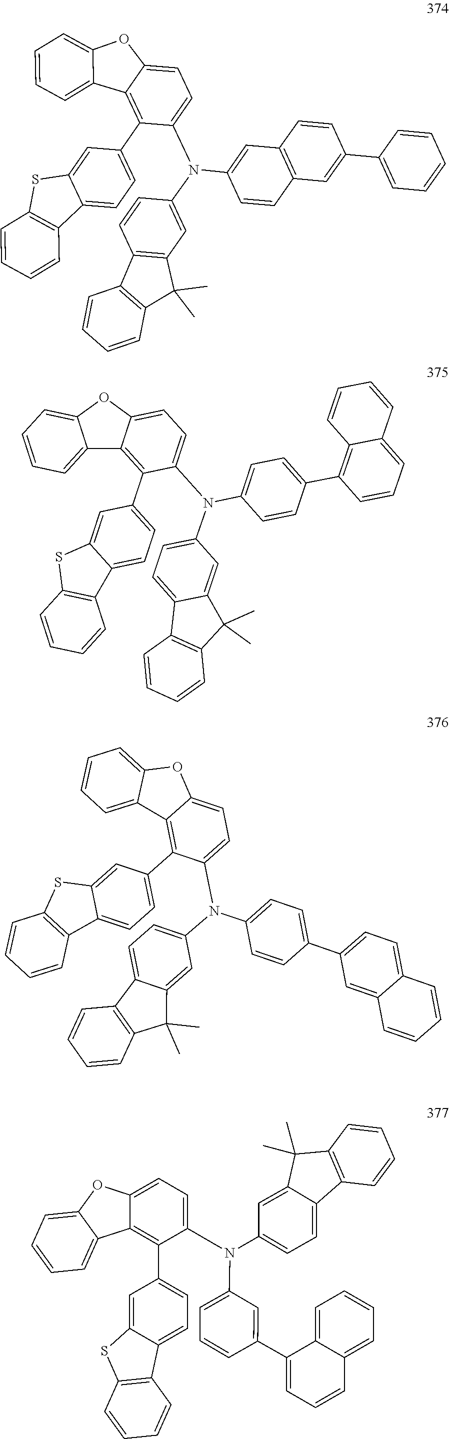

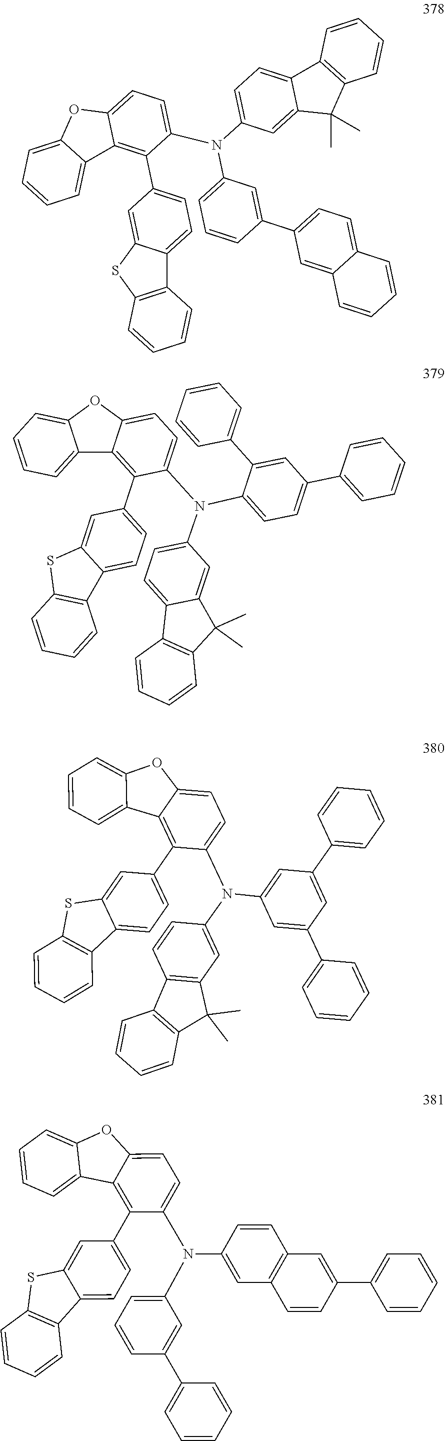 [SMILES: CC1(C)c2ccccc2-c2ccc(N(c3cccc(-c4ccc5ccccc5c4)c3)c3ccc4oc5ccccc5c4c3-c3ccc4c(c3)sc3ccccc34)cc21 ; CC1(C)c2ccccc2-c2ccc(N(c3ccc(-c4ccccc4)cc3-c3ccccc3)c3ccc4oc5ccccc5c4c3-c3ccc4c(c3)sc3ccccc34)cc21 ; CC1(C)c2ccccc2-c2ccc(N(c3cc(-c4ccccc4)cc(-c4ccccc4)c3)c3ccc4oc5ccccc5c4c3-c3ccc4c(c3)sc3ccccc34)cc21 ; c1ccc(-c2cccc(N(c3ccc4cc(-c5ccccc5)ccc4c3)c3ccc4oc5ccccc5c4c3-c3ccc4c(c3)sc3ccccc34)c2)cc1]

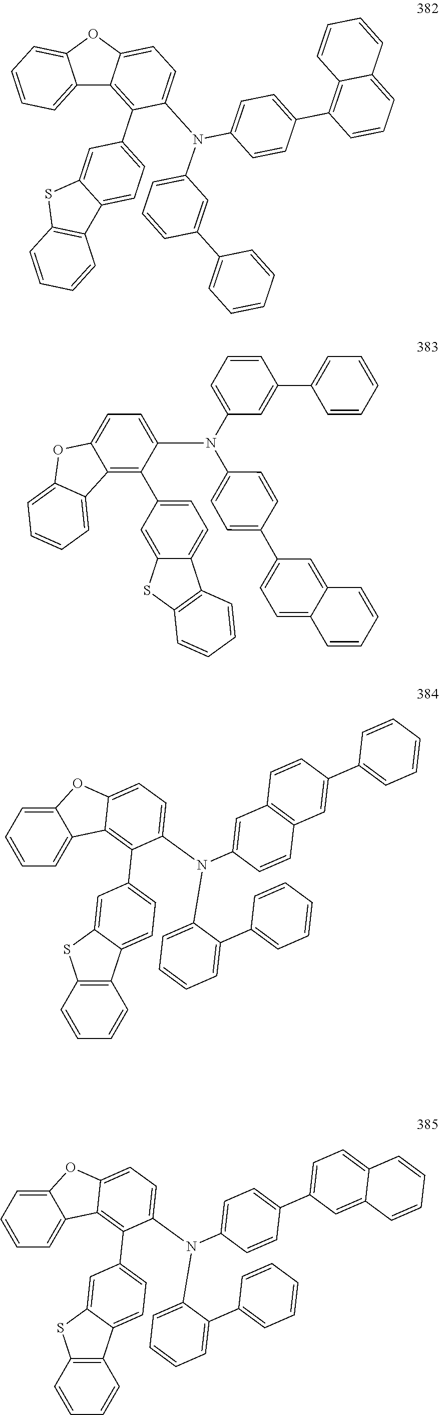

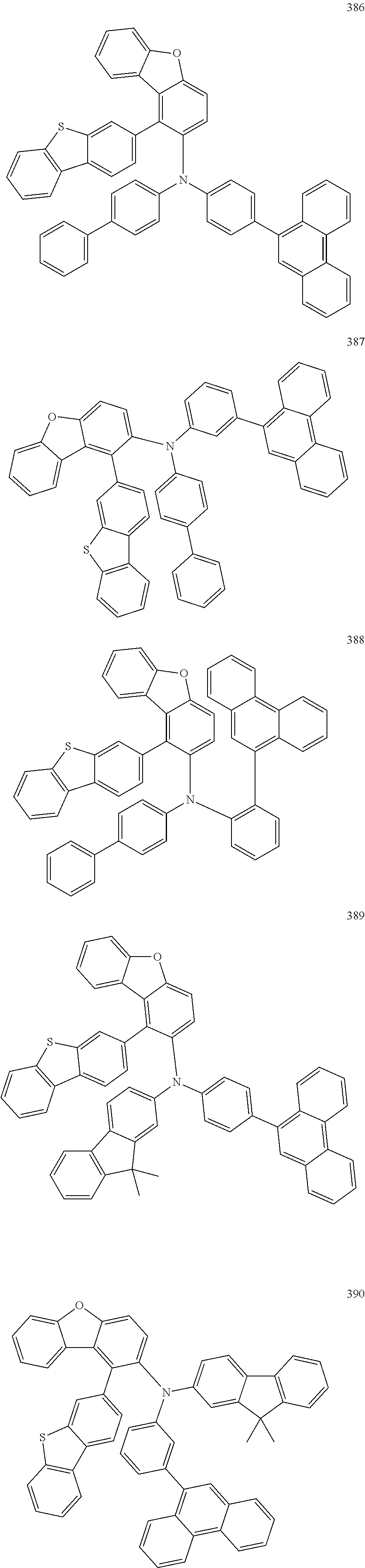

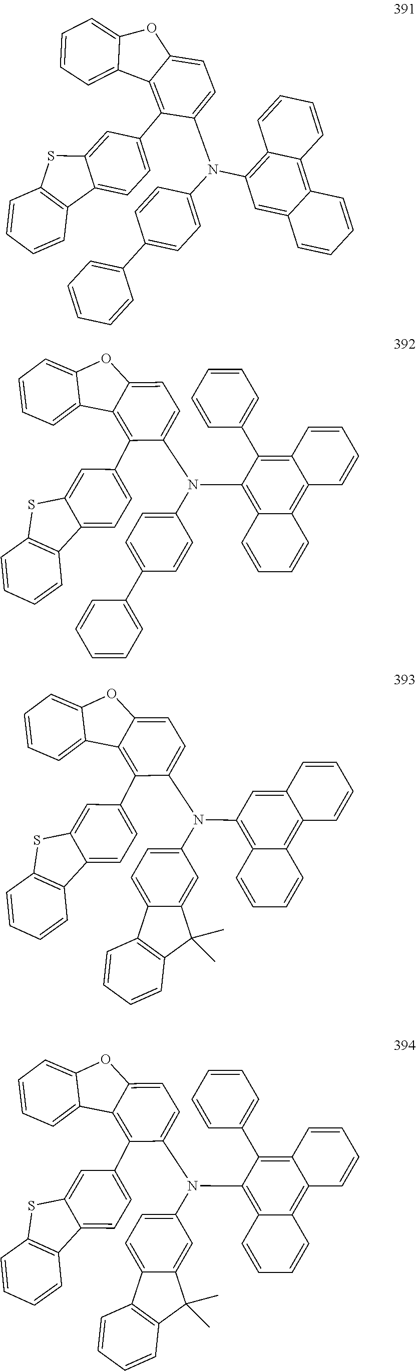

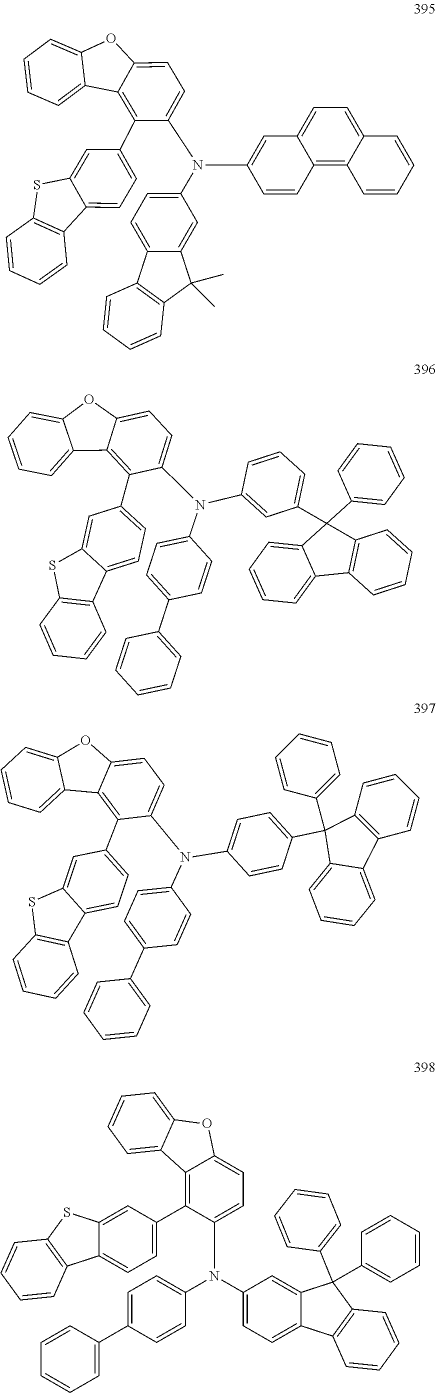 [SMILES: CC1(C)c2ccccc2-c2ccc(N(c3ccc4c(ccc5ccccc54)c3)c3ccc4oc5ccccc5c4c3-c3ccc4c(c3)sc3ccccc34)cc21 ; c1ccc(-c2ccc(N(c3cccc(C4(c5ccccc5)c5ccccc5-c5ccccc54)c3)c3ccc4oc5ccccc5c4c3-c3ccc4c(c3)sc3ccccc34)cc2)cc1 ; c1ccc(-c2ccc(N(c3ccc(C4(c5ccccc5)c5ccccc5-c5ccccc54)cc3)c3ccc4oc5ccccc5c4c3-c3ccc4c(c3)sc3ccccc34)cc2)cc1 ; c1ccc(-c2ccc(N(c3ccc4c(c3)C(c3ccccc3)(c3ccccc3)c3ccccc3-4)c3ccc4oc5ccccc5c4c3-c3ccc4c(c3)sc3ccccc34)cc2)cc1]

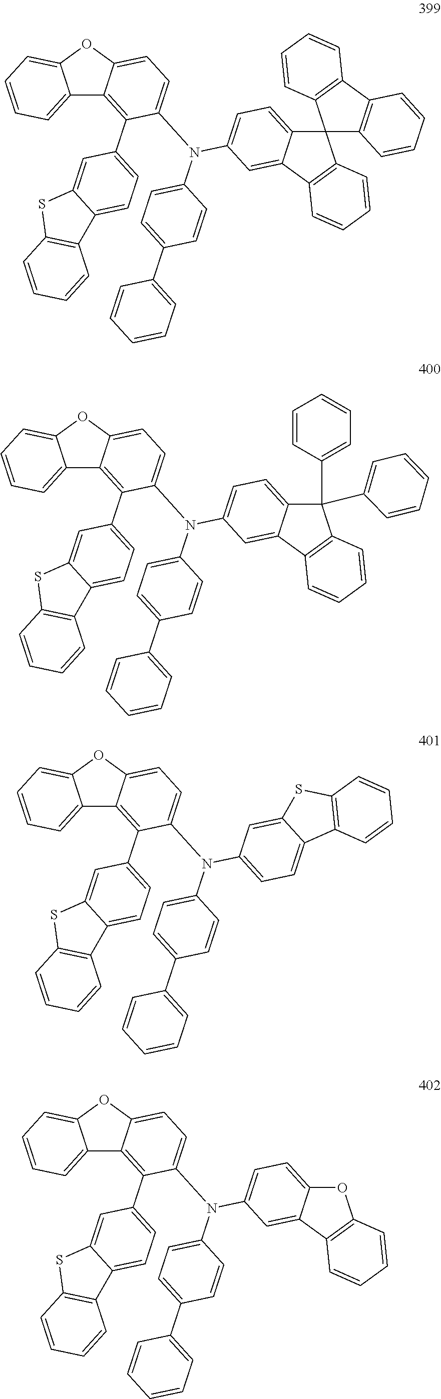 [SMILES: c1ccc(-c2ccc(N(c3ccc4c(c3)-c3ccccc3C43c4ccccc4-c4ccccc43)c3ccc4oc5ccccc5c4c3-c3ccc4c(c3)sc3ccccc34)cc2)cc1 ; c1ccc(-c2ccc(N(c3ccc4c(c3)-c3ccccc3C4(c3ccccc3)c3ccccc3)c3ccc4oc5ccccc5c4c3-c3ccc4c(c3)sc3ccccc34)cc2)cc1 ; c1ccc(-c2ccc(N(c3ccc4c(c3)sc3ccccc34)c3ccc4oc5ccccc5c4c3-c3ccc4c(c3)sc3ccccc34)cc2)cc1 ; c1ccc(-c2ccc(N(c3ccc4oc5ccccc5c4c3)c3ccc4oc5ccccc5c4c3-c3ccc4c(c3)sc3ccccc34)cc2)cc1]

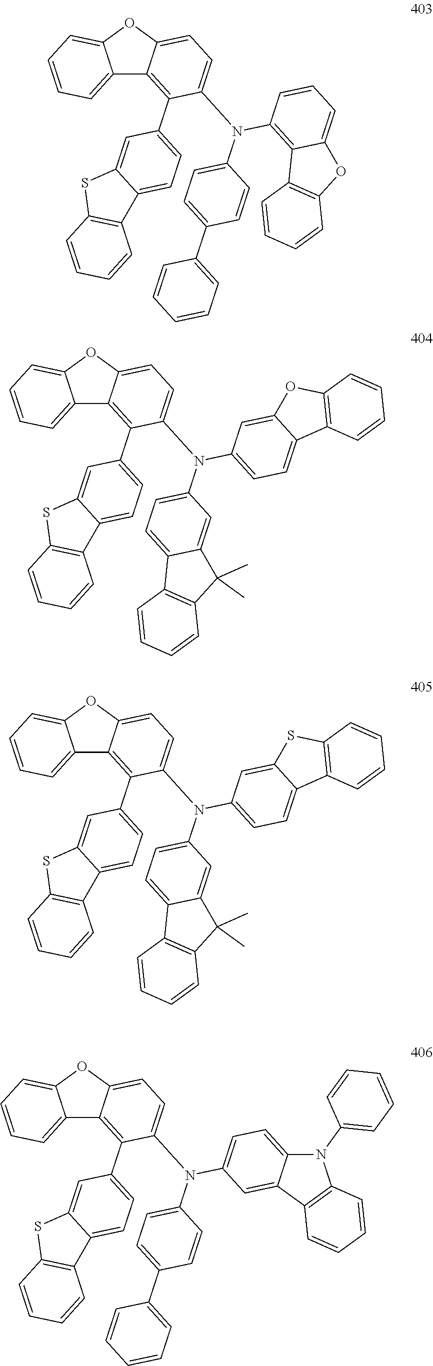 [SMILES: c1ccc(-c2ccc(N(c3ccc4oc5ccccc5c4c3-c3ccc4c(c3)sc3ccccc34)c3cccc4oc5ccccc5c34)cc2)cc1 ; CC1(C)c2ccccc2-c2ccc(N(c3ccc4c(c3)oc3ccccc34)c3ccc4oc5ccccc5c4c3-c3ccc4c(c3)sc3ccccc34)cc21 ; CC1(C)c2ccccc2-c2ccc(N(c3ccc4c(c3)sc3ccccc34)c3ccc4oc5ccccc5c4c3-c3ccc4c(c3)sc3ccccc34)cc21 ; c1ccc(-c2ccc(N(c3ccc4c(c3)c3ccccc3n4-c3ccccc3)c3ccc4oc5ccccc5c4c3-c3ccc4c(c3)sc3ccccc34)cc2)cc1]

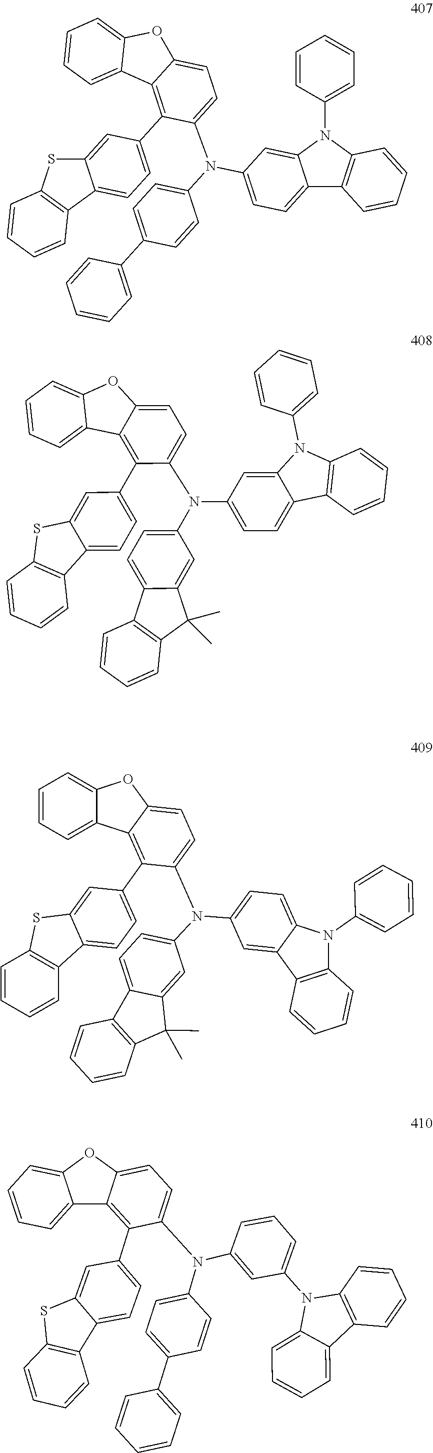 [SMILES: c1ccc(-c2ccc(N(c3ccc4c5ccccc5n(-c5ccccc5)c4c3)c3ccc4oc5ccccc5c4c3-c3ccc4c(c3)sc3ccccc34)cc2)cc1 ; CC1(C)c2ccccc2-c2ccc(N(c3ccc4c5ccccc5n(-c5ccccc5)c4c3)c3ccc4oc5ccccc5c4c3-c3ccc4c(c3)sc3ccccc34)cc21 ; CC1(C)c2ccccc2-c2ccc(N(c3ccc4c(c3)c3ccccc3n4-c3ccccc3)c3ccc4oc5ccccc5c4c3-c3ccc4c(c3)sc3ccccc34)cc21 ; c1ccc(-c2ccc(N(c3cccc(-n4c5ccccc5c5ccccc54)c3)c3ccc4oc5ccccc5c4c3-c3ccc4c(c3)sc3ccccc34)cc2)cc1]

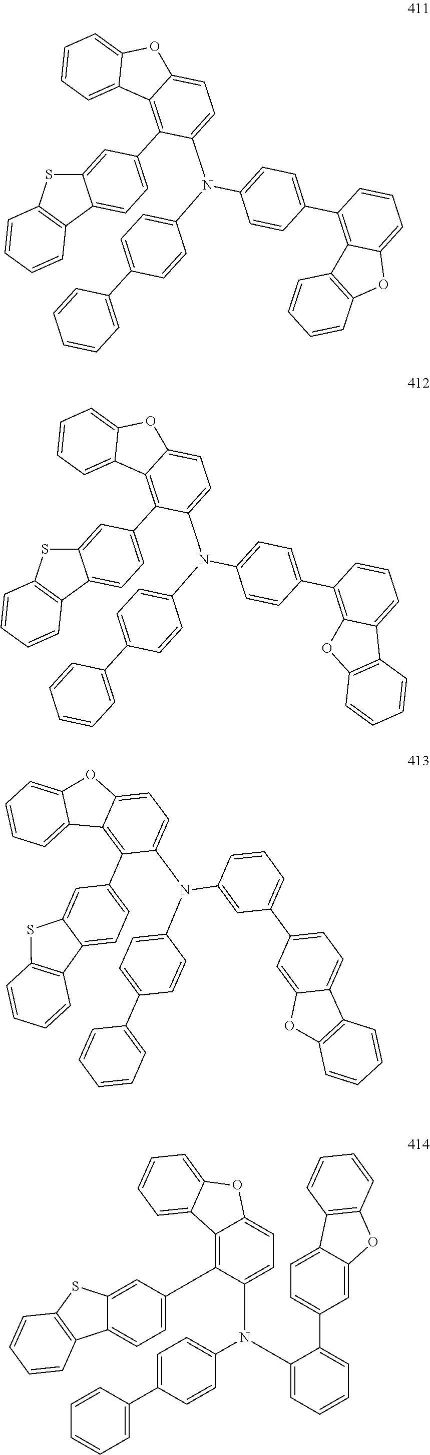 [SMILES: c1ccc(-c2ccc(N(c3ccc(-c4cccc5oc6ccccc6c45)cc3)c3ccc4oc5ccccc5c4c3-c3ccc4c(c3)sc3ccccc34)cc2)cc1 ; c1ccc(-c2ccc(N(c3ccc(-c4cccc5c4oc4ccccc45)cc3)c3ccc4oc5ccccc5c4c3-c3ccc4c(c3)sc3ccccc34)cc2)cc1 ; c1ccc(-c2ccc(N(c3cccc(-c4ccc5c(c4)oc4ccccc45)c3)c3ccc4oc5ccccc5c4c3-c3ccc4c(c3)sc3ccccc34)cc2)cc1 ; c1ccc(-c2ccc(N(c3ccccc3-c3ccc4c(c3)oc3ccccc34)c3ccc4oc5ccccc5c4c3-c3ccc4c(c3)sc3ccccc34)cc2)cc1]

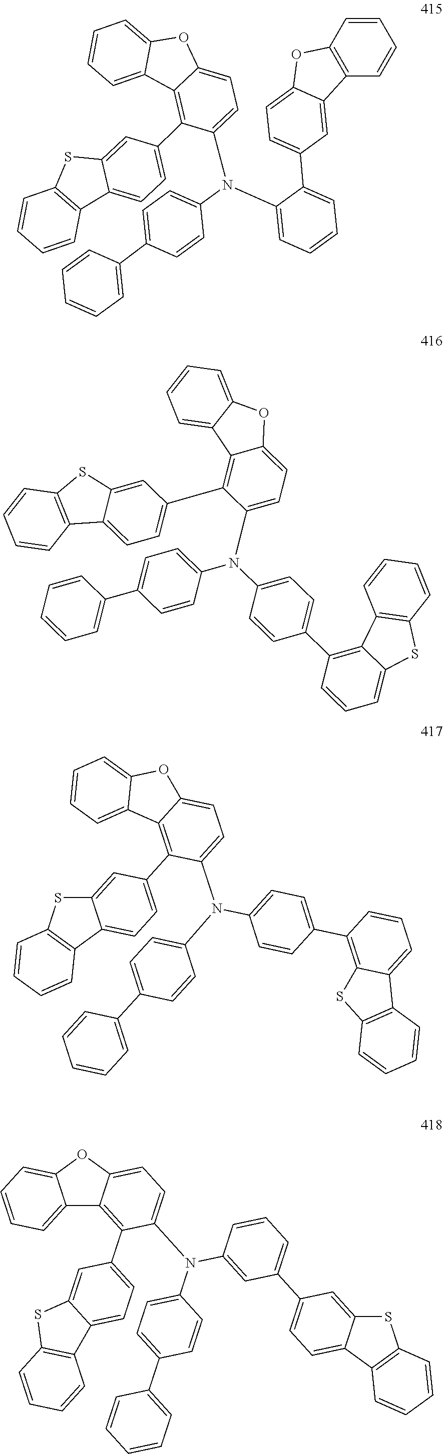 [SMILES: c1ccc(-c2ccc(N(c3ccccc3-c3ccc4oc5ccccc5c4c3)c3ccc4oc5ccccc5c4c3-c3ccc4c(c3)sc3ccccc34)cc2)cc1 ; c1ccc(-c2ccc(N(c3ccc(-c4cccc5sc6ccccc6c45)cc3)c3ccc4oc5ccccc5c4c3-c3ccc4c(c3)sc3ccccc34)cc2)cc1 ; c1ccc(-c2ccc(N(c3ccc(-c4cccc5c4sc4ccccc45)cc3)c3ccc4oc5ccccc5c4c3-c3ccc4c(c3)sc3ccccc34)cc2)cc1 ; c1ccc(-c2ccc(N(c3cccc(-c4ccc5c(c4)sc4ccccc45)c3)c3ccc4oc5ccccc5c4c3-c3ccc4c(c3)sc3ccccc34)cc2)cc1]

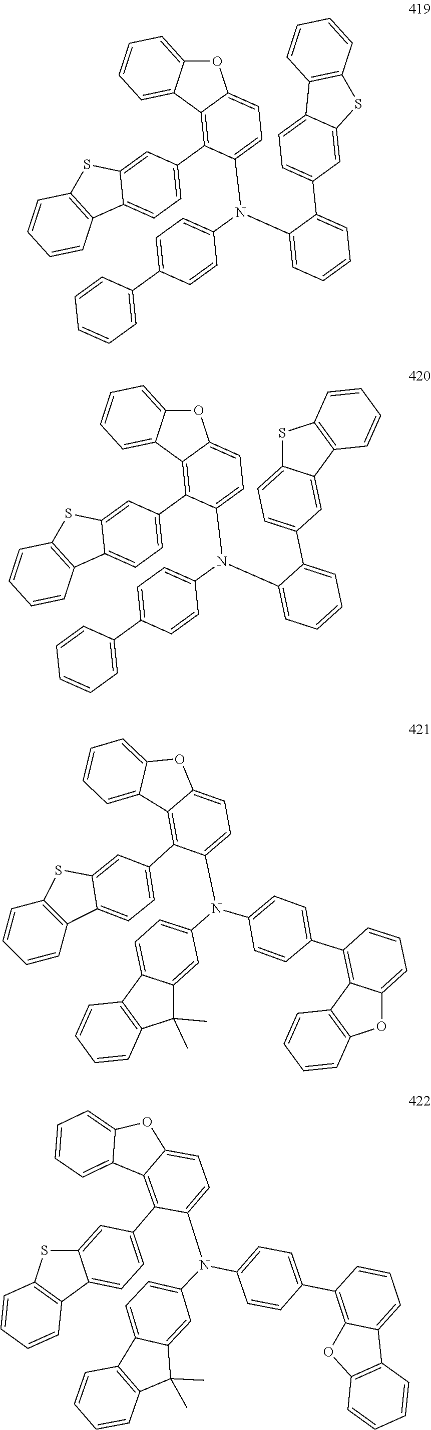 [SMILES: c1ccc(-c2ccc(N(c3ccccc3-c3ccc4c(c3)sc3ccccc34)c3ccc4oc5ccccc5c4c3-c3ccc4c(c3)sc3ccccc34)cc2)cc1 ; c1ccc(-c2ccc(N(c3ccccc3-c3ccc4sc5ccccc5c4c3)c3ccc4oc5ccccc5c4c3-c3ccc4c(c3)sc3ccccc34)cc2)cc1 ; CC1(C)c2ccccc2-c2ccc(N(c3ccc(-c4cccc5oc6ccccc6c45)cc3)c3ccc4oc5ccccc5c4c3-c3ccc4c(c3)sc3ccccc34)cc21 ; CC1(C)c2ccccc2-c2ccc(N(c3ccc(-c4cccc5c4oc4ccccc45)cc3)c3ccc4oc5ccccc5c4c3-c3ccc4c(c3)sc3ccccc34)cc21]

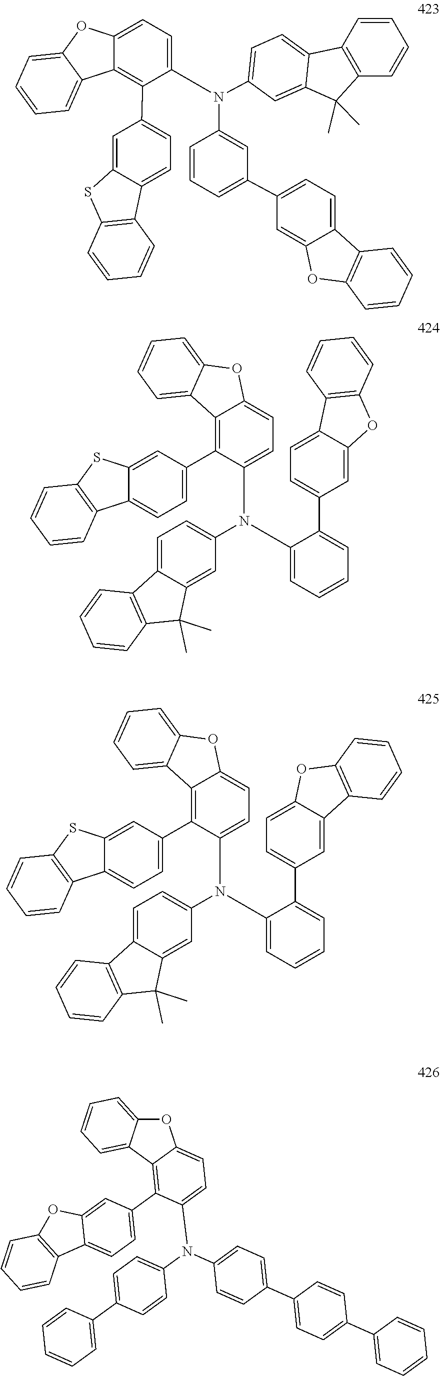

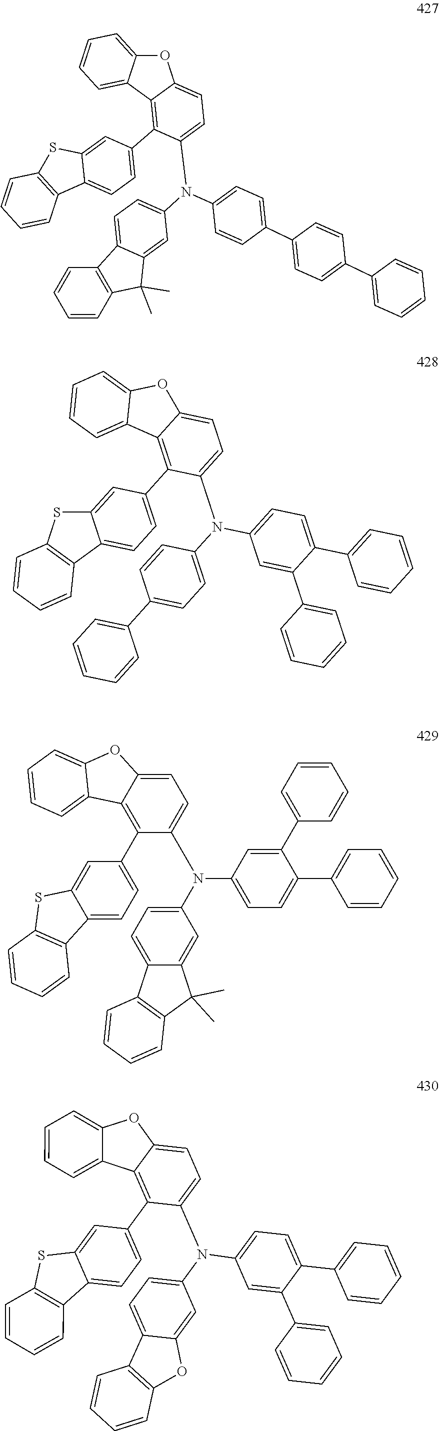 [SMILES: CC1(C)c2ccccc2-c2ccc(N(c3ccc(-c4ccc(-c5ccccc5)cc4)cc3)c3ccc4oc5ccccc5c4c3-c3ccc4c(c3)sc3ccccc34)cc21 ; c1ccc(-c2ccc(N(c3ccc(-c4ccccc4)c(-c4ccccc4)c3)c3ccc4oc5ccccc5c4c3-c3ccc4c(c3)sc3ccccc34)cc2)cc1 ; CC1(C)c2ccccc2-c2ccc(N(c3ccc(-c4ccccc4)c(-c4ccccc4)c3)c3ccc4oc5ccccc5c4c3-c3ccc4c(c3)sc3ccccc34)cc21 ; c1ccc(-c2ccc(N(c3ccc4c(c3)oc3ccccc34)c3ccc4oc5ccccc5c4c3-c3ccc4c(c3)sc3ccccc34)cc2-c2ccccc2)cc1]

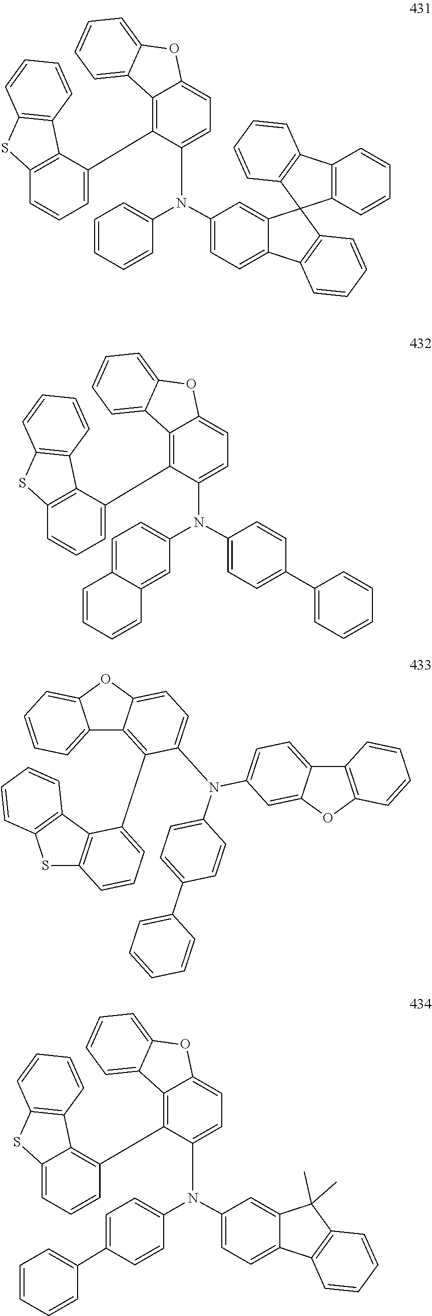

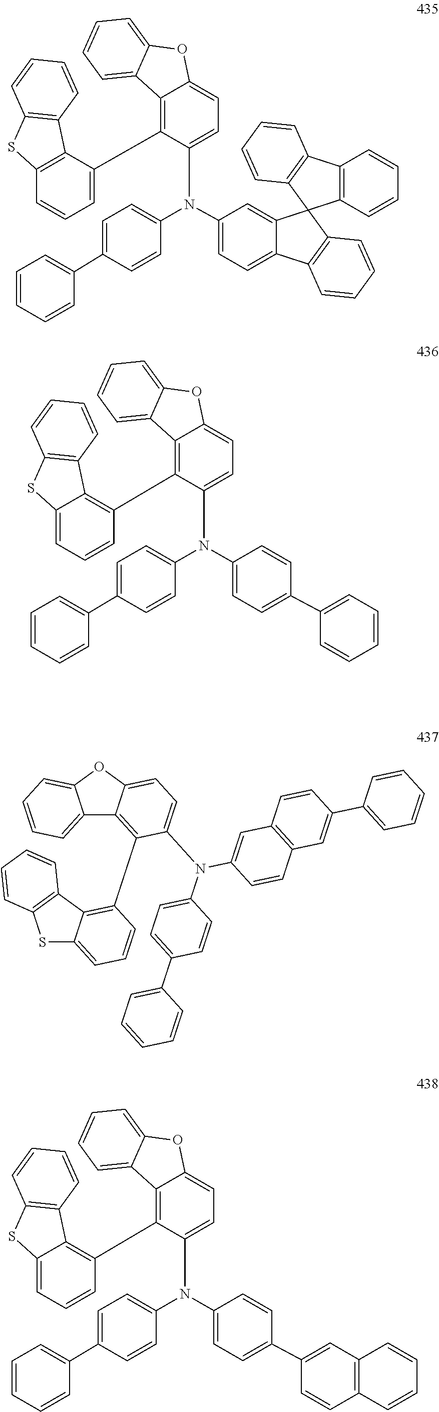 [SMILES: c1ccc(-c2ccc(N(c3ccc4c(c3)C3(c5ccccc5-c5ccccc53)c3ccccc3-4)c3ccc4oc5ccccc5c4c3-c3cccc4sc5ccccc5c34)cc2)cc1 ; c1ccc(-c2ccc(N(c3ccc(-c4ccccc4)cc3)c3ccc4oc5ccccc5c4c3-c3cccc4sc5ccccc5c34)cc2)cc1 ; c1ccc(-c2ccc(N(c3ccc4cc(-c5ccccc5)ccc4c3)c3ccc4oc5ccccc5c4c3-c3cccc4sc5ccccc5c34)cc2)cc1 ; c1ccc(-c2ccc(N(c3ccc(-c4ccc5ccccc5c4)cc3)c3ccc4oc5ccccc5c4c3-c3cccc4sc5ccccc5c34)cc2)cc1]

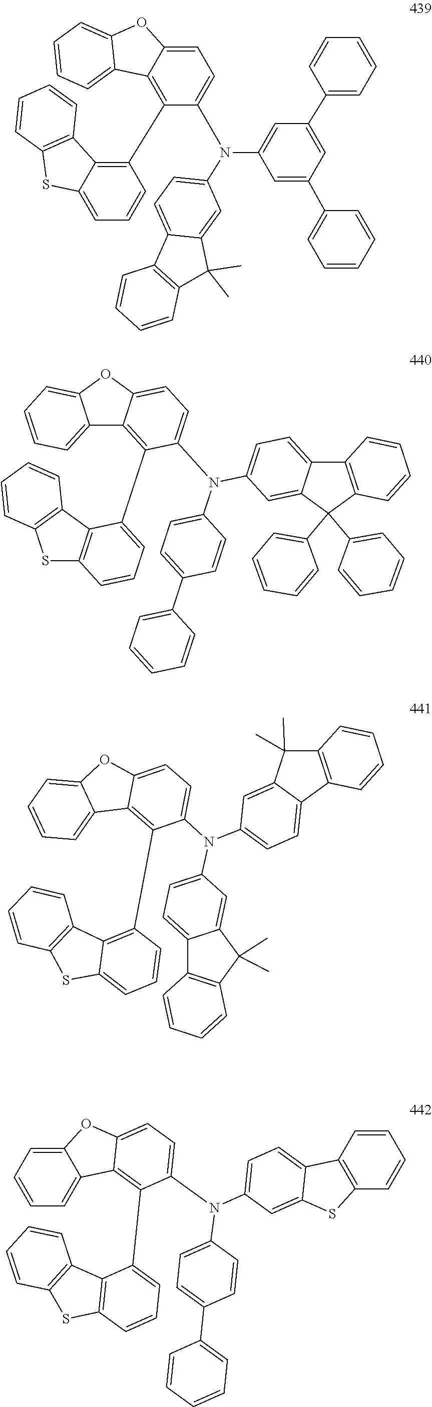 [SMILES: CC1(C)c2ccccc2-c2ccc(N(c3cc(-c4ccccc4)cc(-c4ccccc4)c3)c3ccc4oc5ccccc5c4c3-c3cccc4sc5ccccc5c34)cc21 ; c1ccc(-c2ccc(N(c3ccc4c(c3)C(c3ccccc3)(c3ccccc3)c3ccccc3-4)c3ccc4oc5ccccc5c4c3-c3cccc4sc5ccccc5c34)cc2)cc1 ; CC1(C)c2ccccc2-c2ccc(N(c3ccc4c(c3)C(C)(C)c3ccccc3-4)c3ccc4oc5ccccc5c4c3-c3cccc4sc5ccccc5c34)cc21 ; c1ccc(-c2ccc(N(c3ccc4c(c3)sc3ccccc34)c3ccc4oc5ccccc5c4c3-c3cccc4sc5ccccc5c34)cc2)cc1]

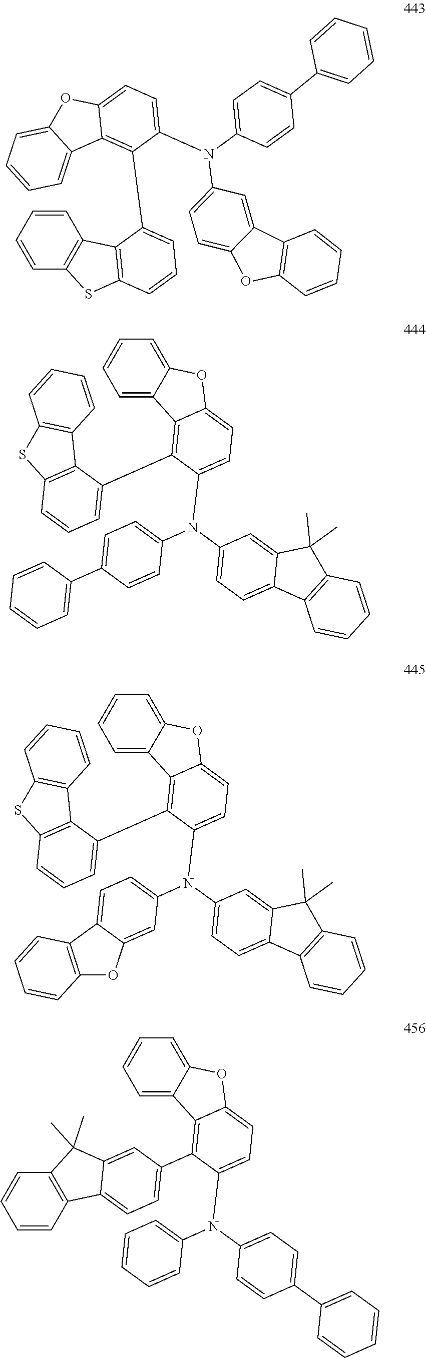

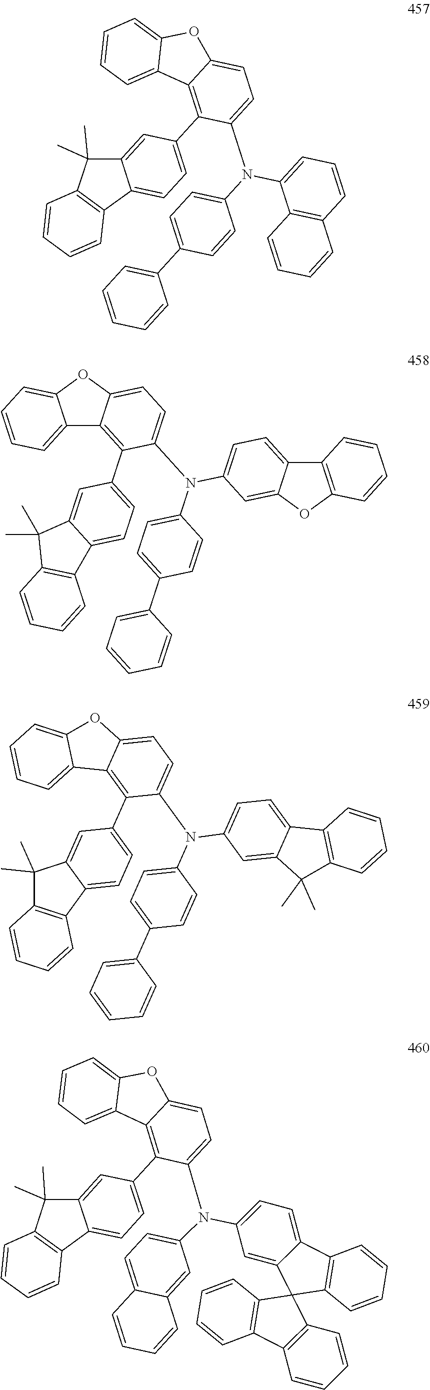

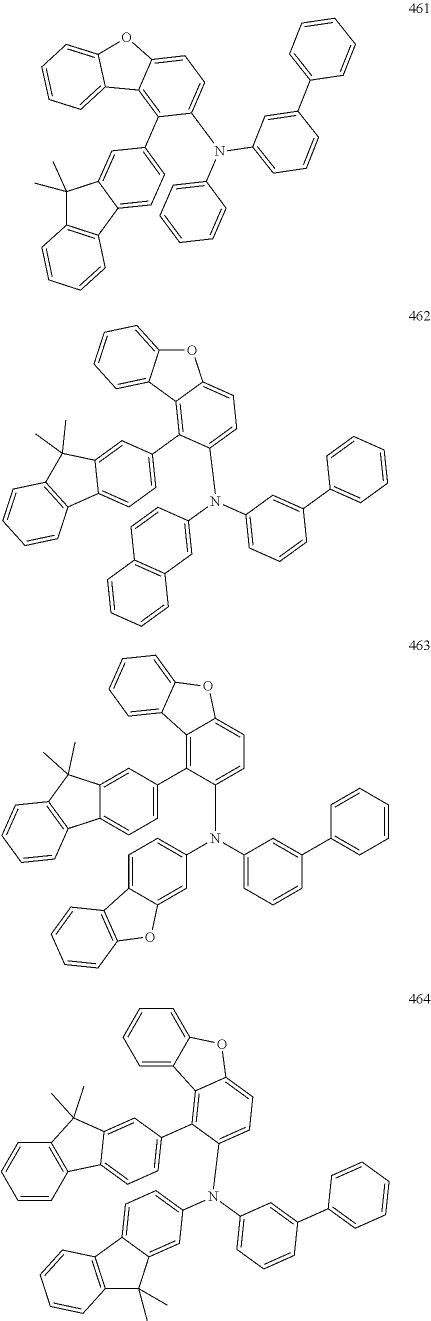

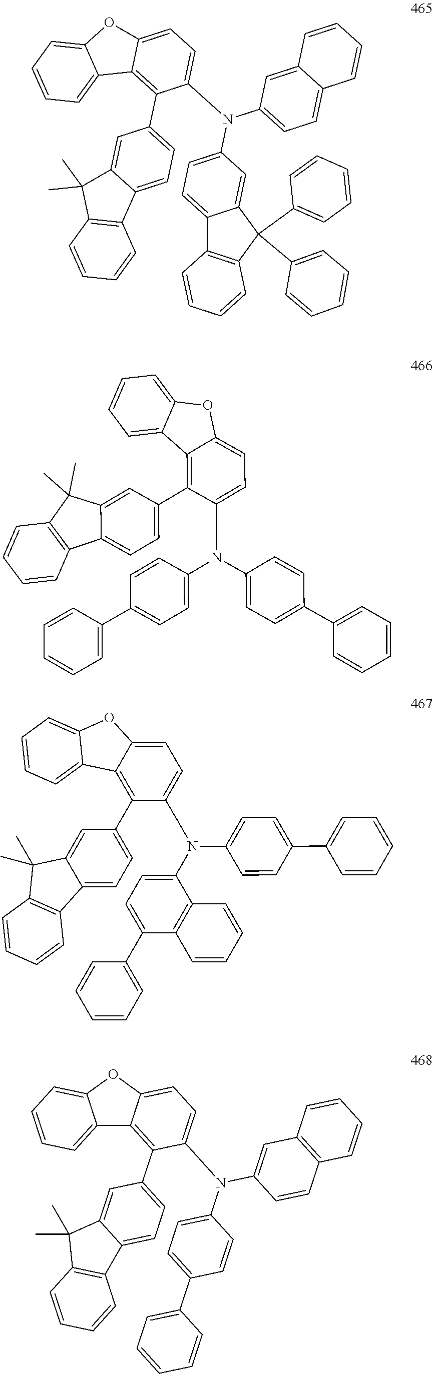 [SMILES: CC1(C)c2ccccc2-c2ccc(-c3c(N(c4ccc5c(c4)C(c4ccccc4)(c4ccccc4)c4ccccc4-5)c4ccc5ccccc5c4)ccc4oc5ccccc5c34)cc21 ; CC1(C)c2ccccc2-c2ccc(-c3c(N(c4ccc(-c5ccccc5)cc4)c4ccc(-c5ccccc5)cc4)ccc4oc5ccccc5c34)cc21 ; CC1(C)c2ccccc2-c2ccc(-c3c(N(c4ccc(-c5ccccc5)cc4)c4ccc(-c5ccccc5)c5ccccc45)ccc4oc5ccccc5c34)cc21 ; CC1(C)c2ccccc2-c2ccc(-c3c(N(c4ccc(-c5ccccc5)cc4)c4ccc5ccccc5c4)ccc4oc5ccccc5c34)cc21]

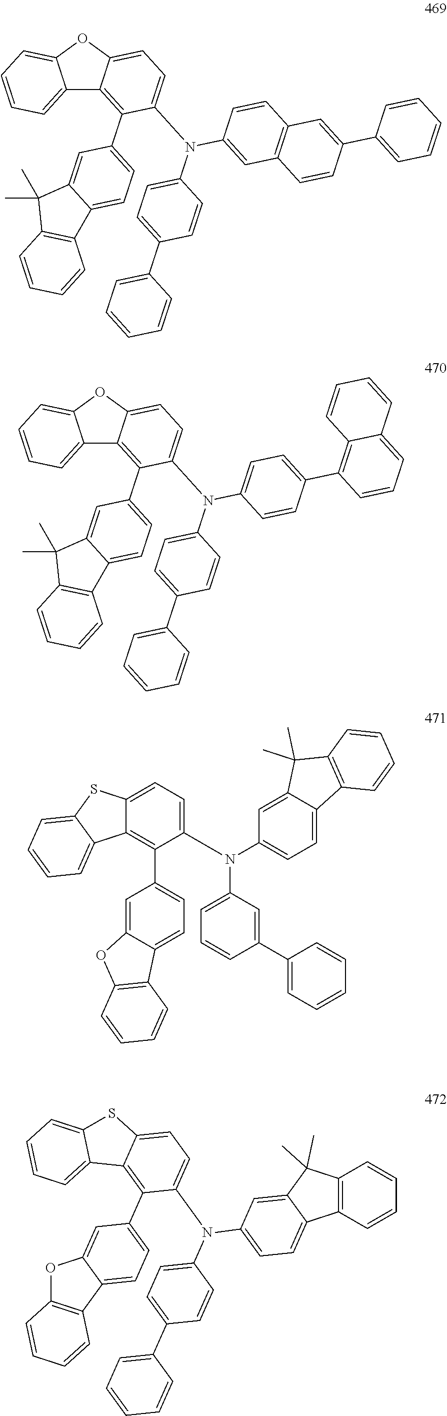 [SMILES: CC1(C)c2ccccc2-c2ccc(-c3c(N(c4ccc(-c5ccccc5)cc4)c4ccc5cc(-c6ccccc6)ccc5c4)ccc4oc5ccccc5c34)cc21 ; CC1(C)c2ccccc2-c2ccc(-c3c(N(c4ccc(-c5ccccc5)cc4)c4ccc(-c5cccc6ccccc56)cc4)ccc4oc5ccccc5c34)cc21 ; CC1(C)c2ccccc2-c2ccc(N(c3cccc(-c4ccccc4)c3)c3ccc4sc5ccccc5c4c3-c3ccc4c(c3)oc3ccccc34)cc21 ; CC1(C)c2ccccc2-c2ccc(N(c3ccc(-c4ccccc4)cc3)c3ccc4sc5ccccc5c4c3-c3ccc4c(c3)oc3ccccc34)cc21]

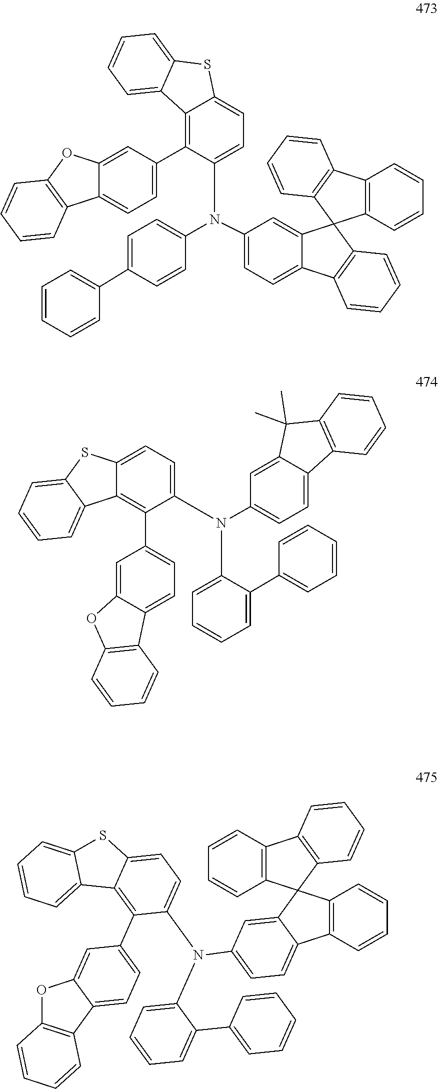 [SMILES: c1ccc(-c2ccc(N(c3ccc4c(c3)C3(c5ccccc5-c5ccccc53)c3ccccc3-4)c3ccc4sc5ccccc5c4c3-c3ccc4c(c3)oc3ccccc34)cc2)cc1 ; CC1(C)c2ccccc2-c2ccc(N(c3ccccc3-c3ccccc3)c3ccc4sc5ccccc5c4c3-c3ccc4c(c3)oc3ccccc34)cc21 ; c1ccc(-c2ccccc2N(c2ccc3c(c2)C2(c4ccccc4-c4ccccc42)c2ccccc2-3)c2ccc3sc4ccccc4c3c2-c2ccc3c(c2)oc2ccccc23)cc1]

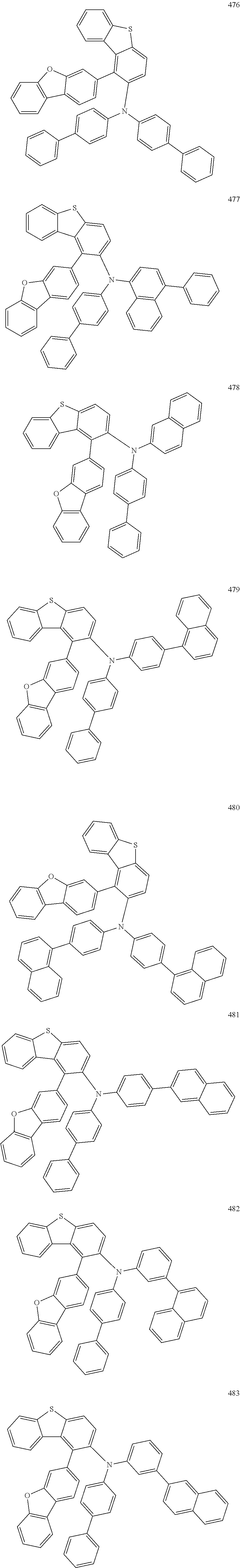

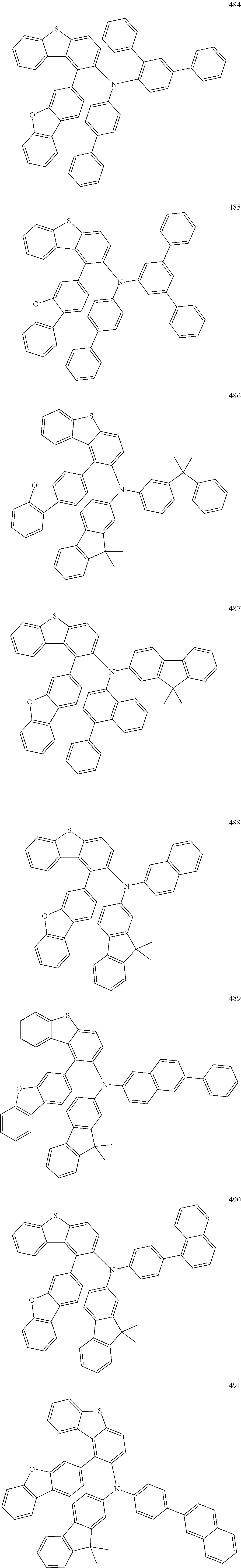

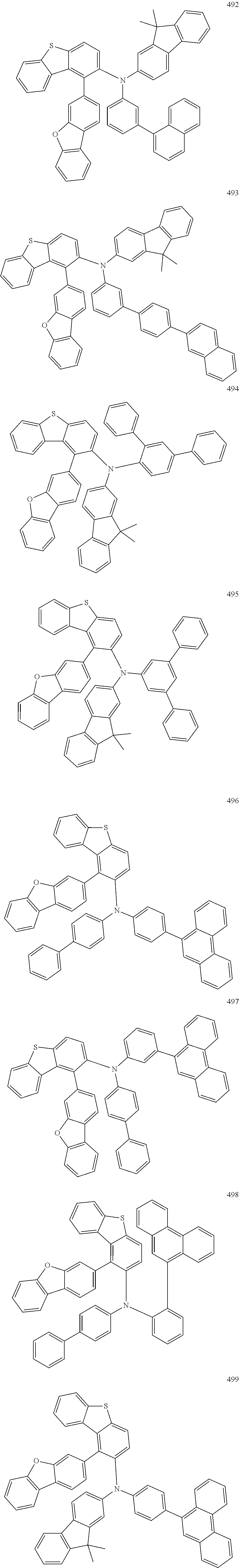

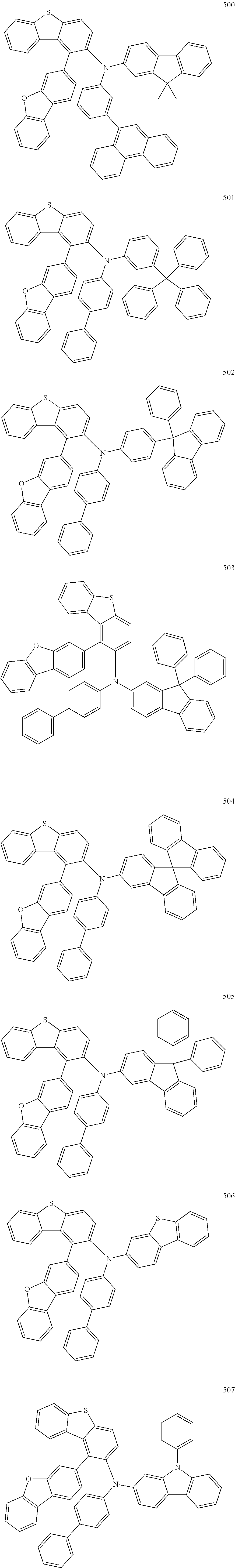

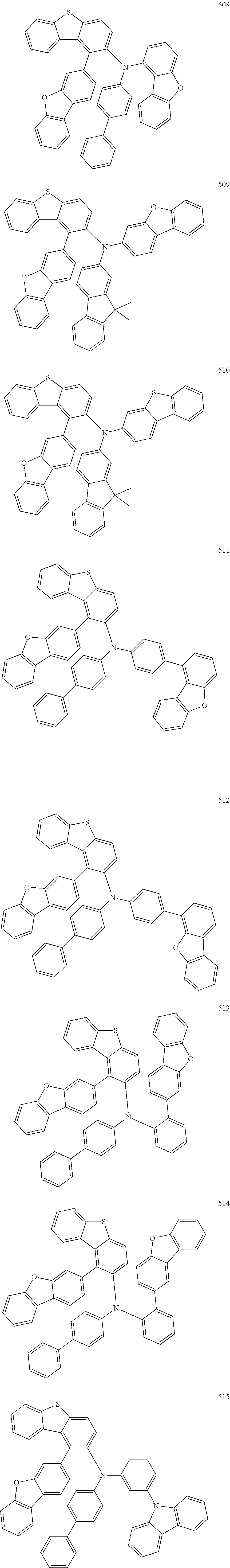

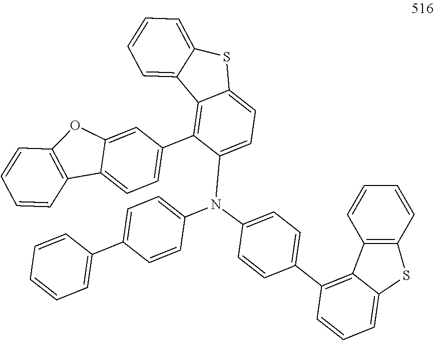 [SMILES: c1ccc(-c2ccc(N(c3ccc(-c4cccc5sc6ccccc6c45)cc3)c3ccc4sc5ccccc5c4c3-c3ccc4c(c3)oc3ccccc34)cc2)cc1]

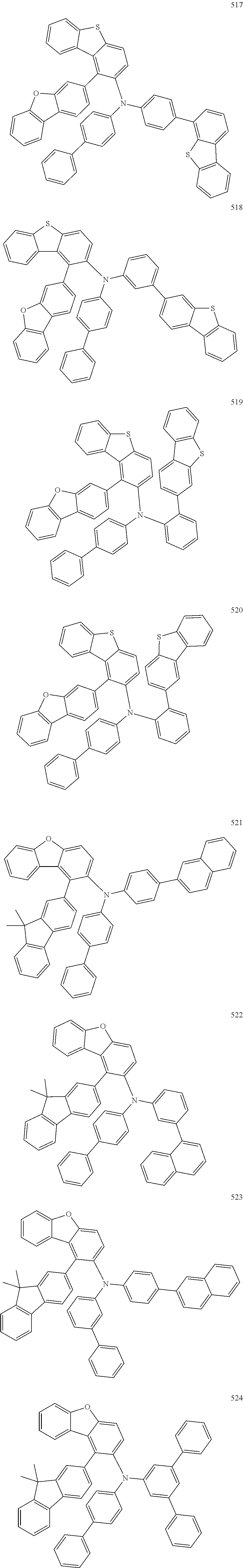

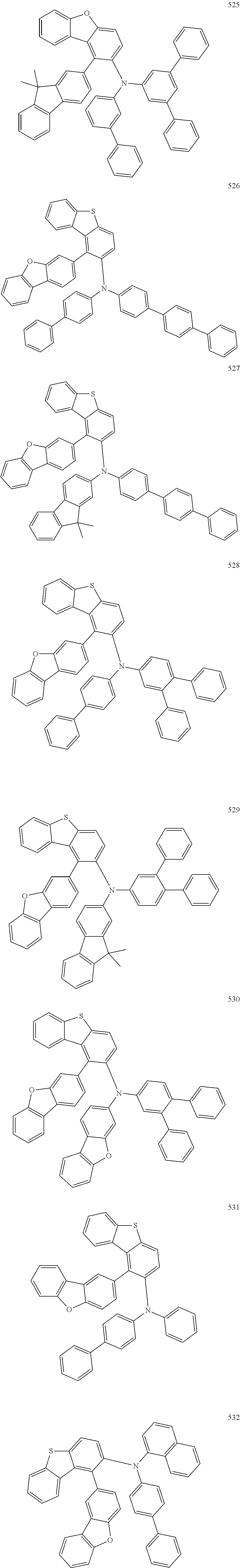

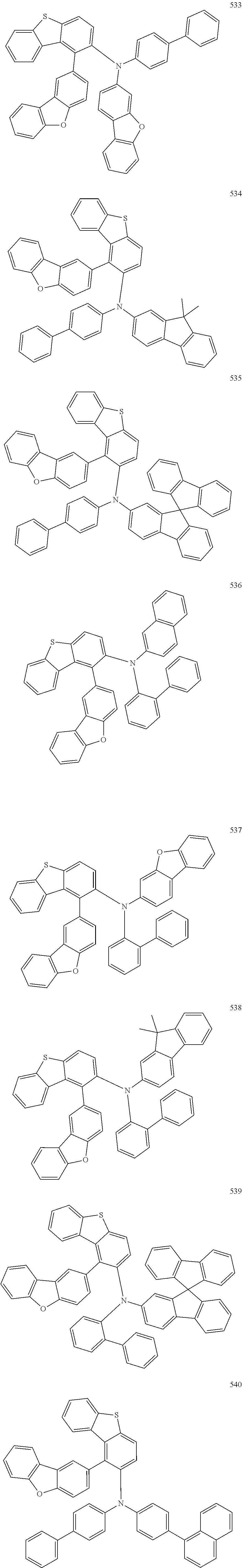

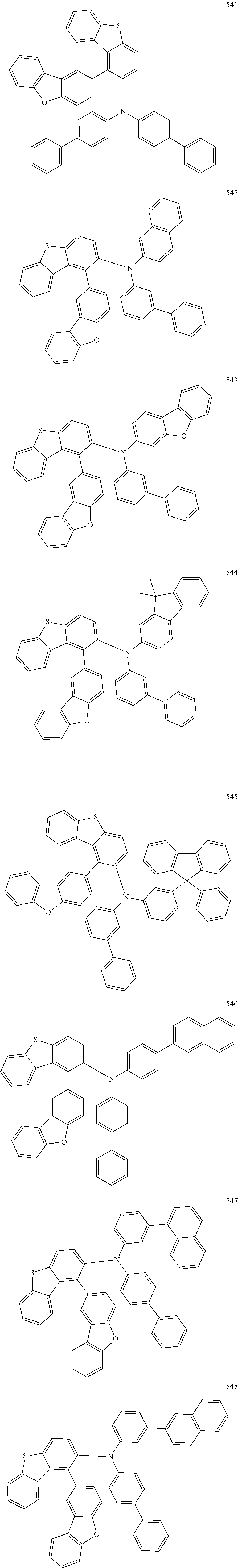

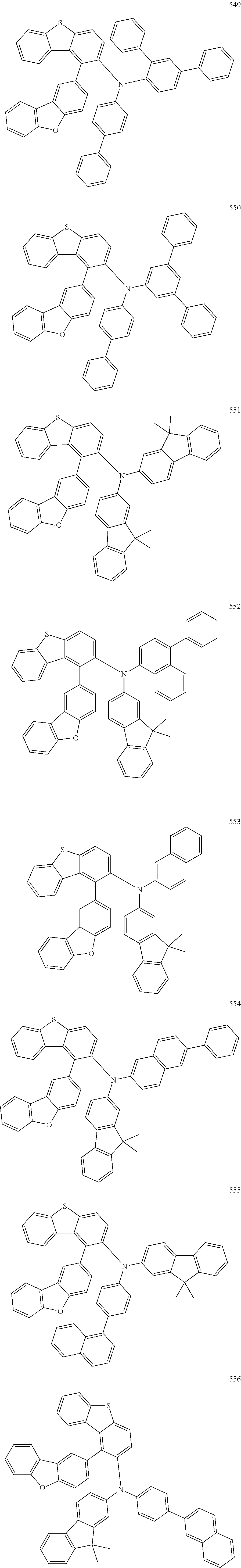

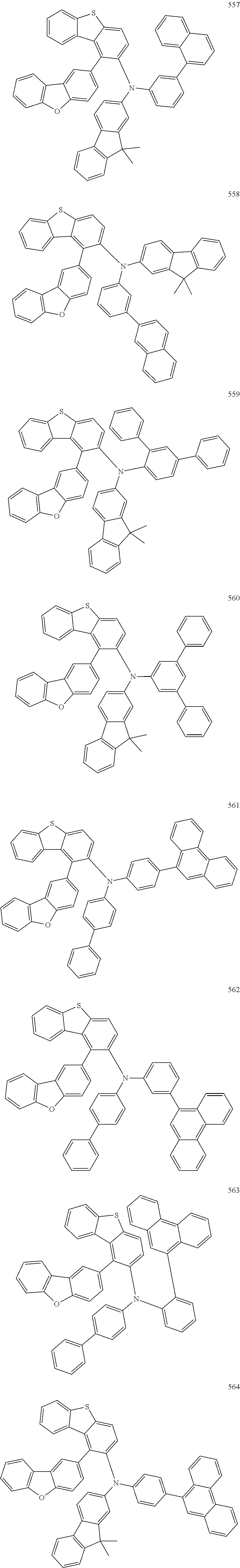

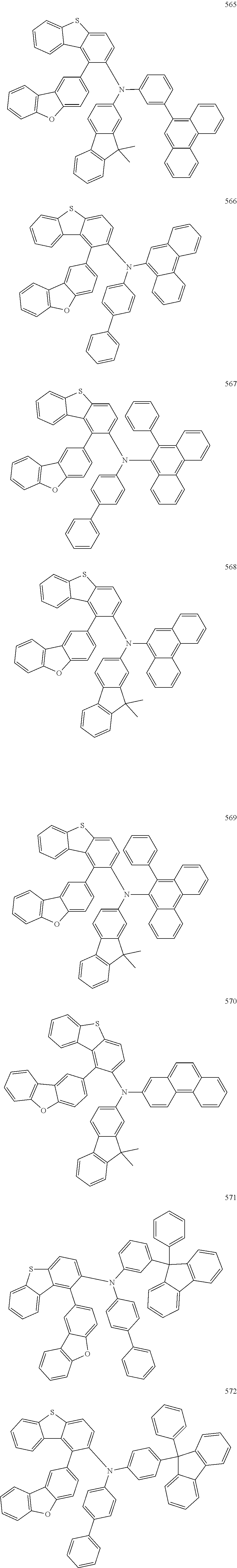

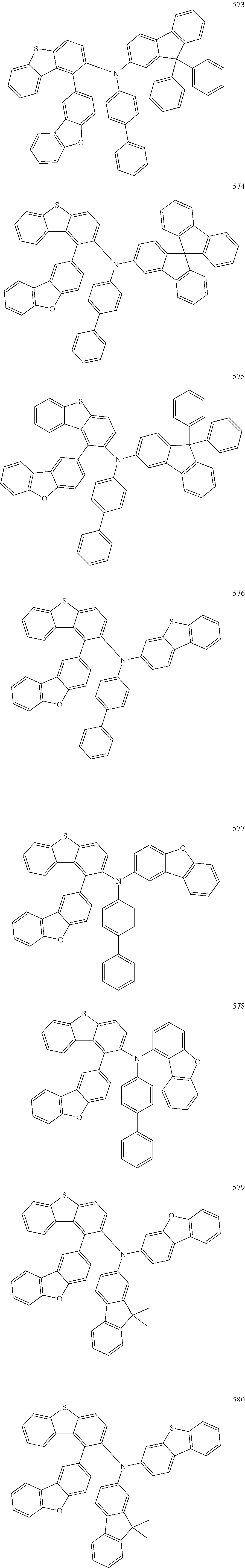

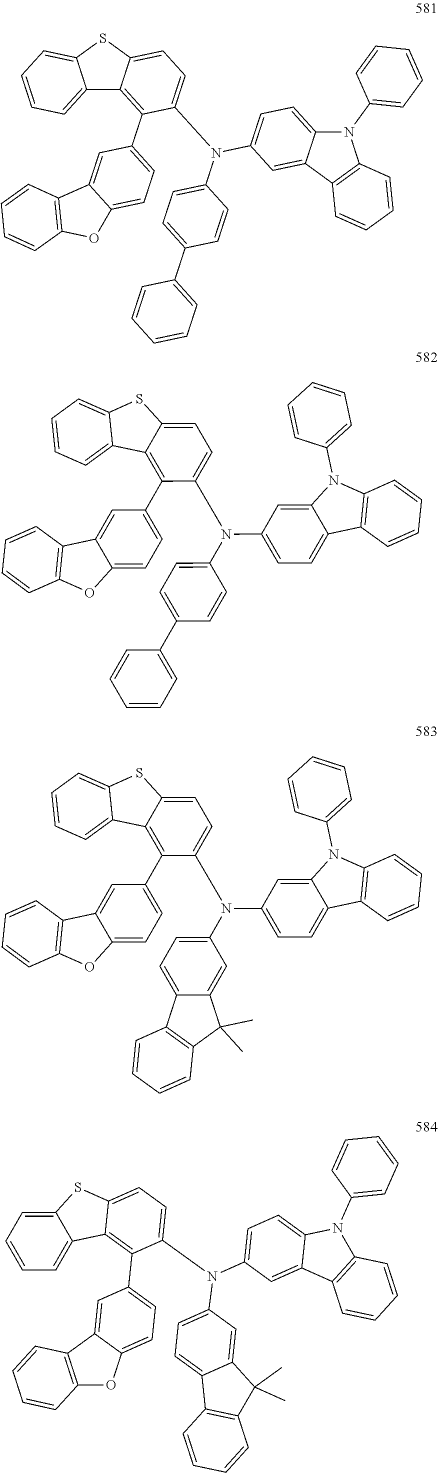 [SMILES: c1ccc(-c2ccc(N(c3ccc4c(c3)c3ccccc3n4-c3ccccc3)c3ccc4sc5ccccc5c4c3-c3ccc4oc5ccccc5c4c3)cc2)cc1 ; c1ccc(-c2ccc(N(c3ccc4c5ccccc5n(-c5ccccc5)c4c3)c3ccc4sc5ccccc5c4c3-c3ccc4oc5ccccc5c4c3)cc2)cc1 ; CC1(C)c2ccccc2-c2ccc(N(c3ccc4c5ccccc5n(-c5ccccc5)c4c3)c3ccc4sc5ccccc5c4c3-c3ccc4oc5ccccc5c4c3)cc21 ; CC1(C)c2ccccc2-c2ccc(N(c3ccc4c(c3)c3ccccc3n4-c3ccccc3)c3ccc4sc5ccccc5c4c3-c3ccc4oc5ccccc5c4c3)cc21]

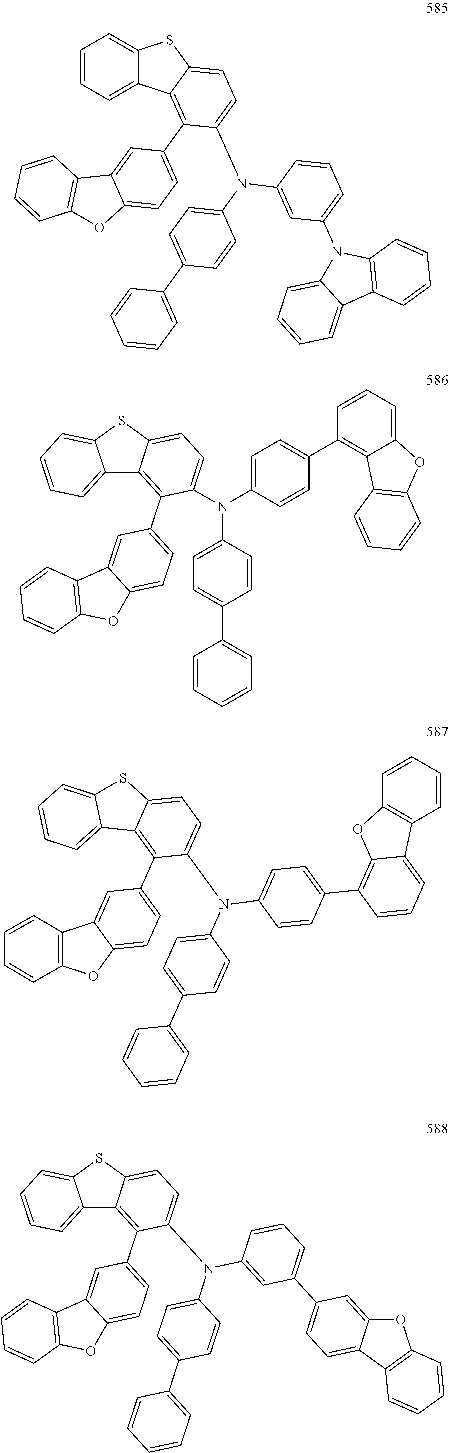 [SMILES: c1ccc(-c2ccc(N(c3cccc(-n4c5ccccc5c5ccccc54)c3)c3ccc4sc5ccccc5c4c3-c3ccc4oc5ccccc5c4c3)cc2)cc1 ; c1ccc(-c2ccc(N(c3ccc(-c4cccc5oc6ccccc6c45)cc3)c3ccc4sc5ccccc5c4c3-c3ccc4oc5ccccc5c4c3)cc2)cc1 ; c1ccc(-c2ccc(N(c3ccc(-c4cccc5c4oc4ccccc45)cc3)c3ccc4sc5ccccc5c4c3-c3ccc4oc5ccccc5c4c3)cc2)cc1 ; c1ccc(-c2ccc(N(c3cccc(-c4ccc5c(c4)oc4ccccc45)c3)c3ccc4sc5ccccc5c4c3-c3ccc4oc5ccccc5c4c3)cc2)cc1]

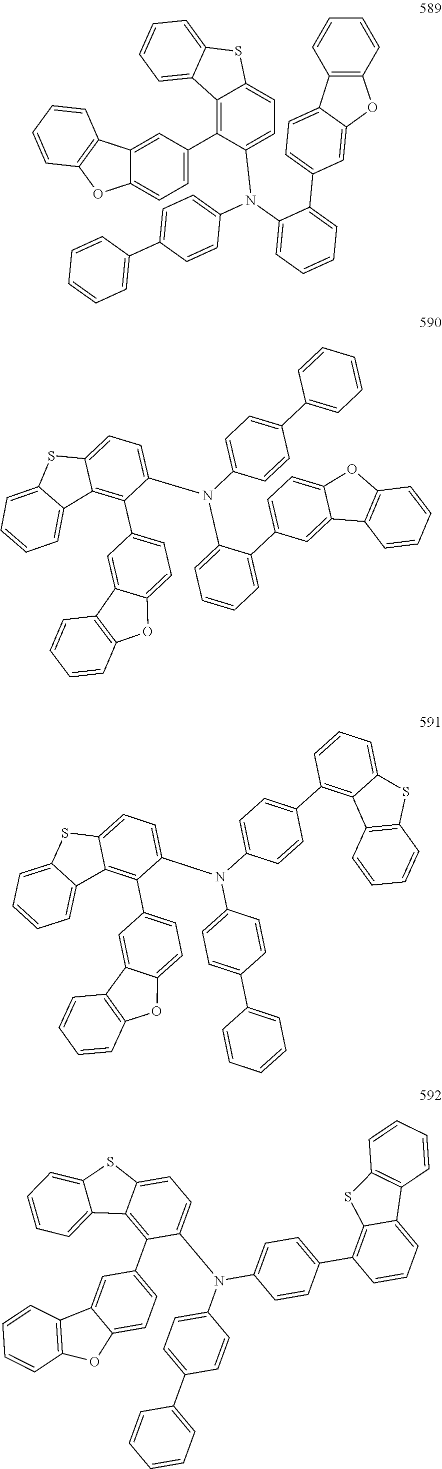 [SMILES: c1ccc(-c2ccc(N(c3ccccc3-c3ccc4c(c3)oc3ccccc34)c3ccc4sc5ccccc5c4c3-c3ccc4oc5ccccc5c4c3)cc2)cc1 ; c1ccc(-c2ccc(N(c3ccccc3-c3ccc4oc5ccccc5c4c3)c3ccc4sc5ccccc5c4c3-c3ccc4oc5ccccc5c4c3)cc2)cc1 ; c1ccc(-c2ccc(N(c3ccc(-c4cccc5sc6ccccc6c45)cc3)c3ccc4sc5ccccc5c4c3-c3ccc4oc5ccccc5c4c3)cc2)cc1 ; c1ccc(-c2ccc(N(c3ccc(-c4cccc5c4sc4ccccc45)cc3)c3ccc4sc5ccccc5c4c3-c3ccc4oc5ccccc5c4c3)cc2)cc1]

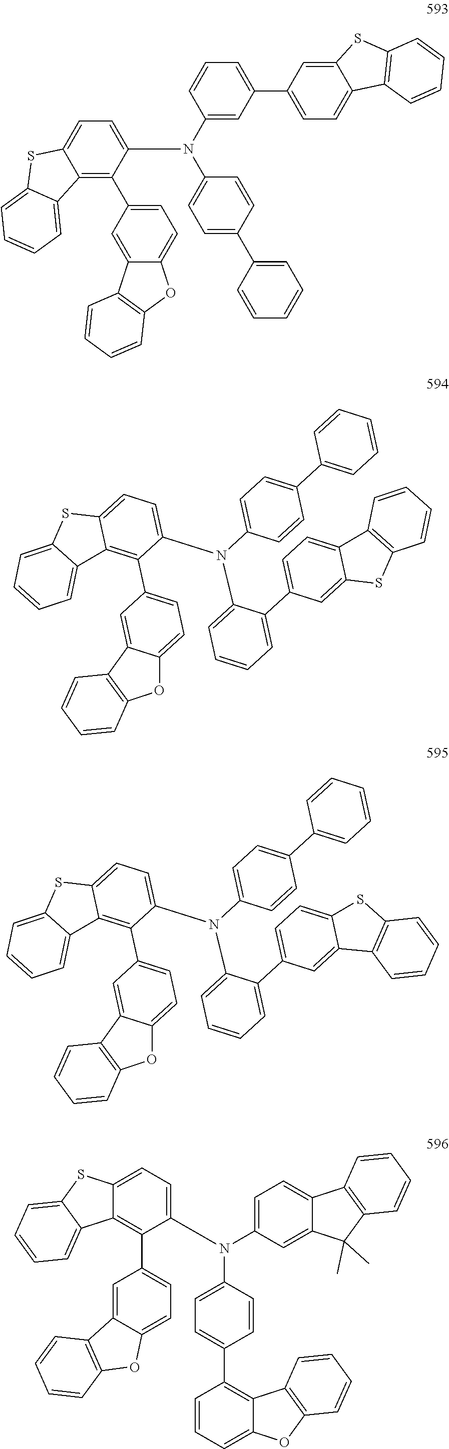 [SMILES: c1ccc(-c2ccc(N(c3cccc(-c4ccc5c(c4)sc4ccccc45)c3)c3ccc4sc5ccccc5c4c3-c3ccc4oc5ccccc5c4c3)cc2)cc1 ; c1ccc(-c2ccc(N(c3ccccc3-c3ccc4c(c3)sc3ccccc34)c3ccc4sc5ccccc5c4c3-c3ccc4oc5ccccc5c4c3)cc2)cc1 ; c1ccc(-c2ccc(N(c3ccccc3-c3ccc4sc5ccccc5c4c3)c3ccc4sc5ccccc5c4c3-c3ccc4oc5ccccc5c4c3)cc2)cc1 ; CC1(C)c2ccccc2-c2ccc(N(c3ccc(-c4cccc5oc6ccccc6c45)cc3)c3ccc4sc5ccccc5c4c3-c3ccc4oc5ccccc5c4c3)cc21]

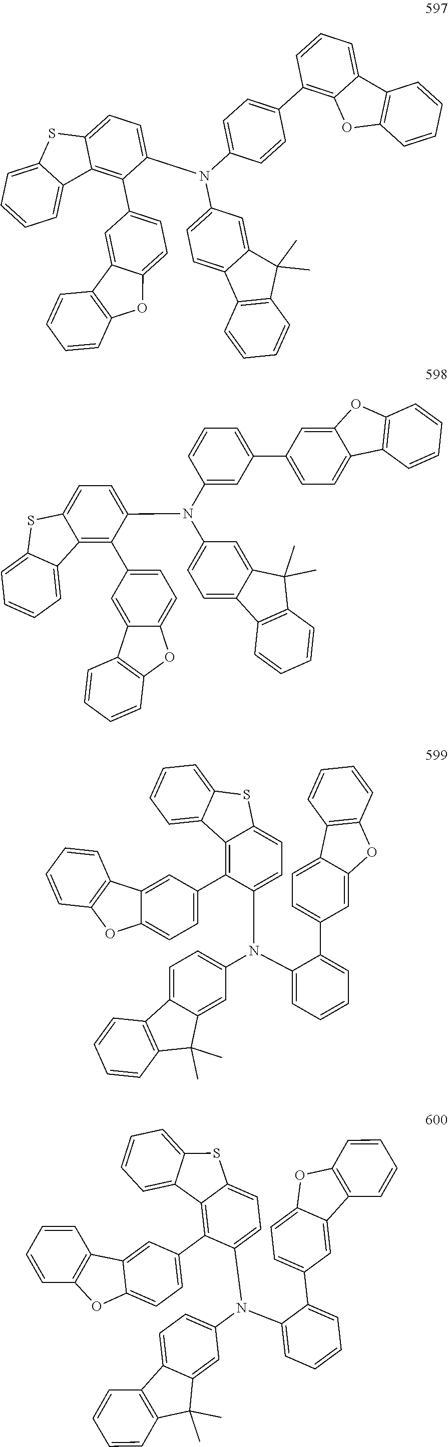 [SMILES: CC1(C)c2ccccc2-c2ccc(N(c3ccc(-c4cccc5c4oc4ccccc45)cc3)c3ccc4sc5ccccc5c4c3-c3ccc4oc5ccccc5c4c3)cc21 ; CC1(C)c2ccccc2-c2ccc(N(c3cccc(-c4ccc5c(c4)oc4ccccc45)c3)c3ccc4sc5ccccc5c4c3-c3ccc4oc5ccccc5c4c3)cc21 ; CC1(C)c2ccccc2-c2ccc(N(c3ccccc3-c3ccc4c(c3)oc3ccccc34)c3ccc4sc5ccccc5c4c3-c3ccc4oc5ccccc5c4c3)cc21 ; CC1(C)c2ccccc2-c2ccc(N(c3ccccc3-c3ccc4oc5ccccc5c4c3)c3ccc4sc5ccccc5c4c3-c3ccc4oc5ccccc5c4c3)cc21]

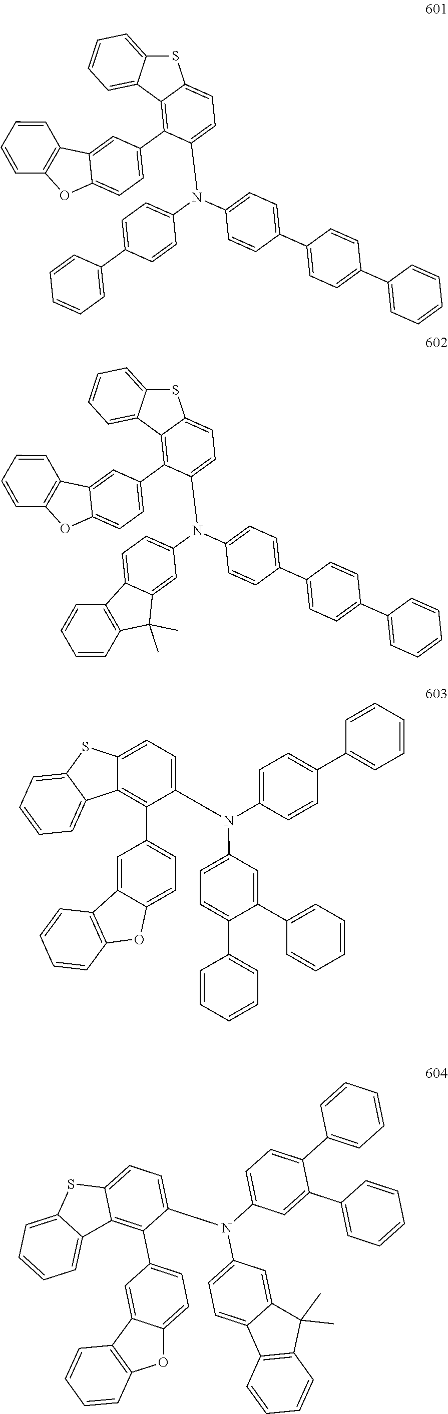 [SMILES: c1ccc(-c2ccc(-c3ccc(N(c4ccc(-c5ccccc5)cc4)c4ccc5sc6ccccc6c5c4-c4ccc5oc6ccccc6c5c4)cc3)cc2)cc1 ; CC1(C)c2ccccc2-c2ccc(N(c3ccc(-c4ccc(-c5ccccc5)cc4)cc3)c3ccc4sc5ccccc5c4c3-c3ccc4oc5ccccc5c4c3)cc21 ; c1ccc(-c2ccc(N(c3ccc(-c4ccccc4)c(-c4ccccc4)c3)c3ccc4sc5ccccc5c4c3-c3ccc4oc5ccccc5c4c3)cc2)cc1 ; CC1(C)c2ccccc2-c2ccc(N(c3ccc(-c4ccccc4)c(-c4ccccc4)c3)c3ccc4sc5ccccc5c4c3-c3ccc4oc5ccccc5c4c3)cc21]

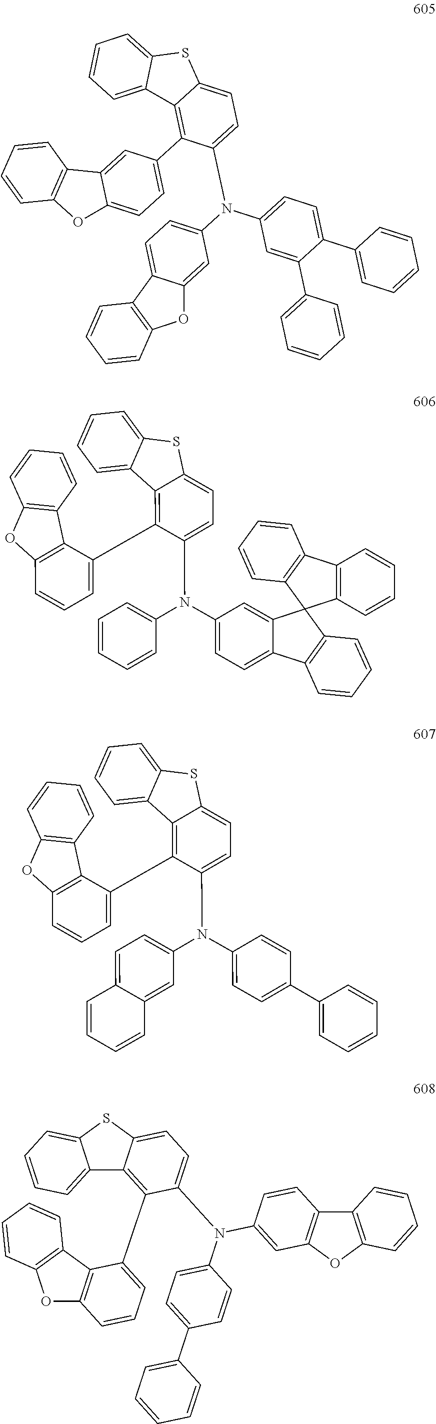 [SMILES: c1ccc(-c2ccc(N(c3ccc4c(c3)oc3ccccc34)c3ccc4sc5ccccc5c4c3-c3ccc4oc5ccccc5c4c3)cc2-c2ccccc2)cc1 ; c1ccc(N(c2ccc3c(c2)C2(c4ccccc4-c4ccccc42)c2ccccc2-3)c2ccc3sc4ccccc4c3c2-c2cccc3oc4ccccc4c23)cc1 ; c1ccc(-c2ccc(N(c3ccc4ccccc4c3)c3ccc4sc5ccccc5c4c3-c3cccc4oc5ccccc5c34)cc2)cc1 ; c1ccc(-c2ccc(N(c3ccc4c(c3)oc3ccccc34)c3ccc4sc5ccccc5c4c3-c3cccc4oc5ccccc5c34)cc2)cc1]

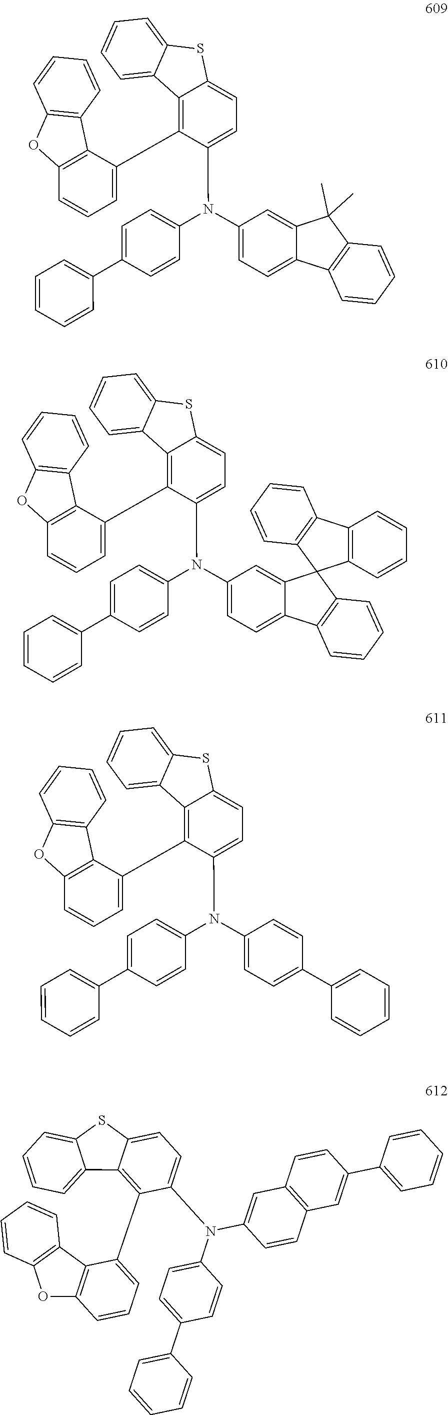 [SMILES: CC1(C)c2ccccc2-c2ccc(N(c3ccc(-c4ccccc4)cc3)c3ccc4sc5ccccc5c4c3-c3cccc4oc5ccccc5c34)cc21 ; c1ccc(-c2ccc(N(c3ccc4c(c3)C3(c5ccccc5-c5ccccc53)c3ccccc3-4)c3ccc4sc5ccccc5c4c3-c3cccc4oc5ccccc5c34)cc2)cc1 ; c1ccc(-c2ccc(N(c3ccc(-c4ccccc4)cc3)c3ccc4sc5ccccc5c4c3-c3cccc4oc5ccccc5c34)cc2)cc1 ; c1ccc(-c2ccc(N(c3ccc4cc(-c5ccccc5)ccc4c3)c3ccc4sc5ccccc5c4c3-c3cccc4oc5ccccc5c34)cc2)cc1]

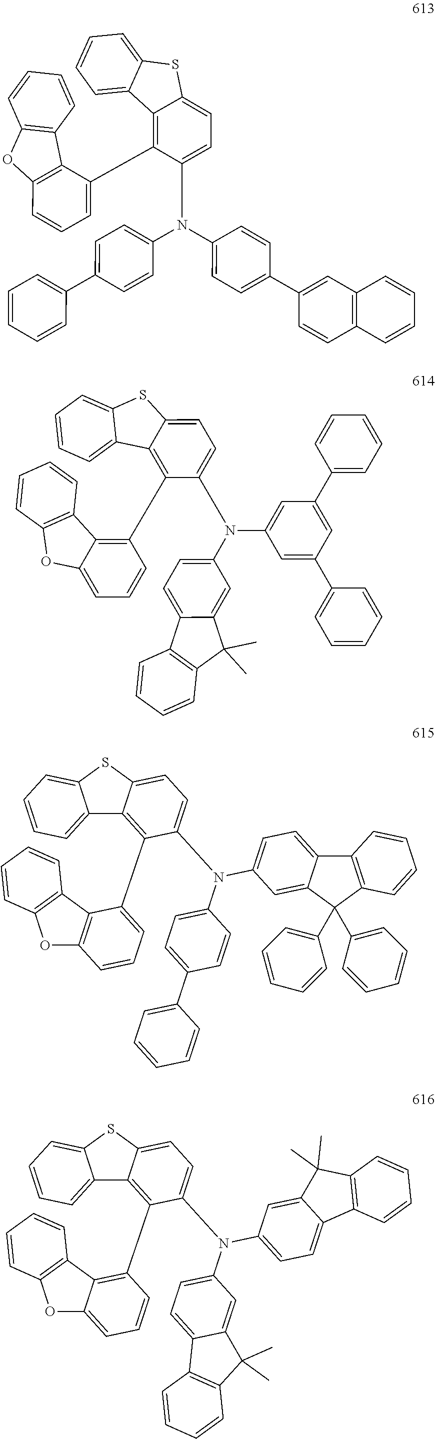 [SMILES: c1ccc(-c2ccc(N(c3ccc(-c4ccc5ccccc5c4)cc3)c3ccc4sc5ccccc5c4c3-c3cccc4oc5ccccc5c34)cc2)cc1 ; CC1(C)c2ccccc2-c2ccc(N(c3cc(-c4ccccc4)cc(-c4ccccc4)c3)c3ccc4sc5ccccc5c4c3-c3cccc4oc5ccccc5c34)cc21 ; c1ccc(-c2ccc(N(c3ccc4c(c3)C(c3ccccc3)(c3ccccc3)c3ccccc3-4)c3ccc4sc5ccccc5c4c3-c3cccc4oc5ccccc5c34)cc2)cc1 ; CC1(C)c2ccccc2-c2ccc(N(c3ccc4c(c3)C(C)(C)c3ccccc3-4)c3ccc4sc5ccccc5c4c3-c3cccc4oc5ccccc5c34)cc21]

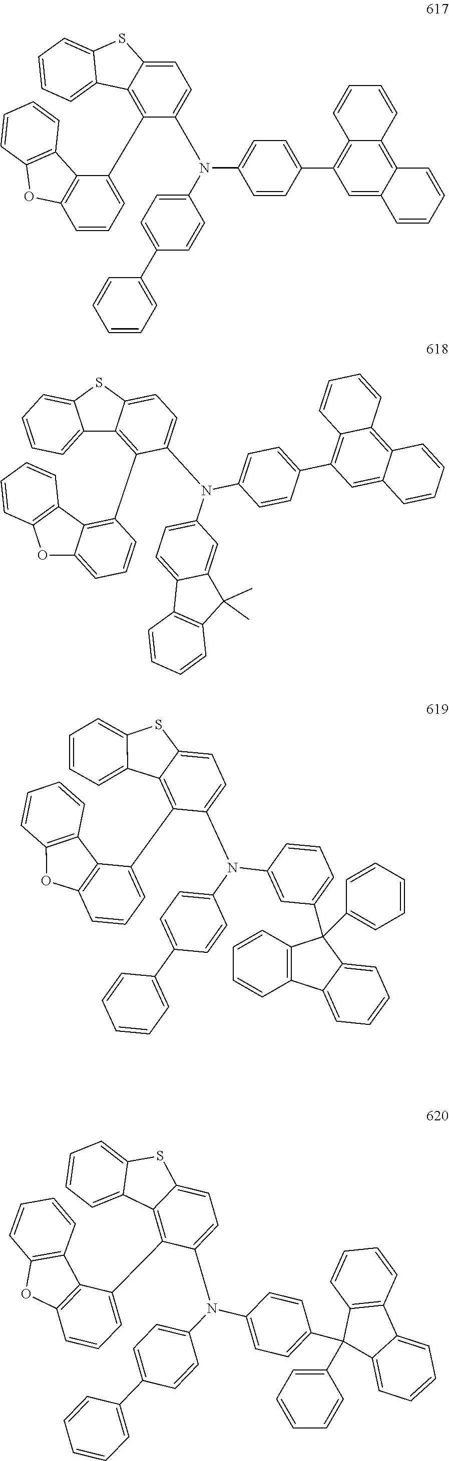 [SMILES: c1ccc(-c2ccc(N(c3ccc(-c4cc5ccccc5c5ccccc45)cc3)c3ccc4sc5ccccc5c4c3-c3cccc4oc5ccccc5c34)cc2)cc1 ; CC1(C)c2ccccc2-c2ccc(N(c3ccc(-c4cc5ccccc5c5ccccc45)cc3)c3ccc4sc5ccccc5c4c3-c3cccc4oc5ccccc5c34)cc21 ; c1ccc(-c2ccc(N(c3cccc(C4(c5ccccc5)c5ccccc5-c5ccccc54)c3)c3ccc4sc5ccccc5c4c3-c3cccc4oc5ccccc5c34)cc2)cc1 ; c1ccc(-c2ccc(N(c3ccc(C4(c5ccccc5)c5ccccc5-c5ccccc54)cc3)c3ccc4sc5ccccc5c4c3-c3cccc4oc5ccccc5c34)cc2)cc1]

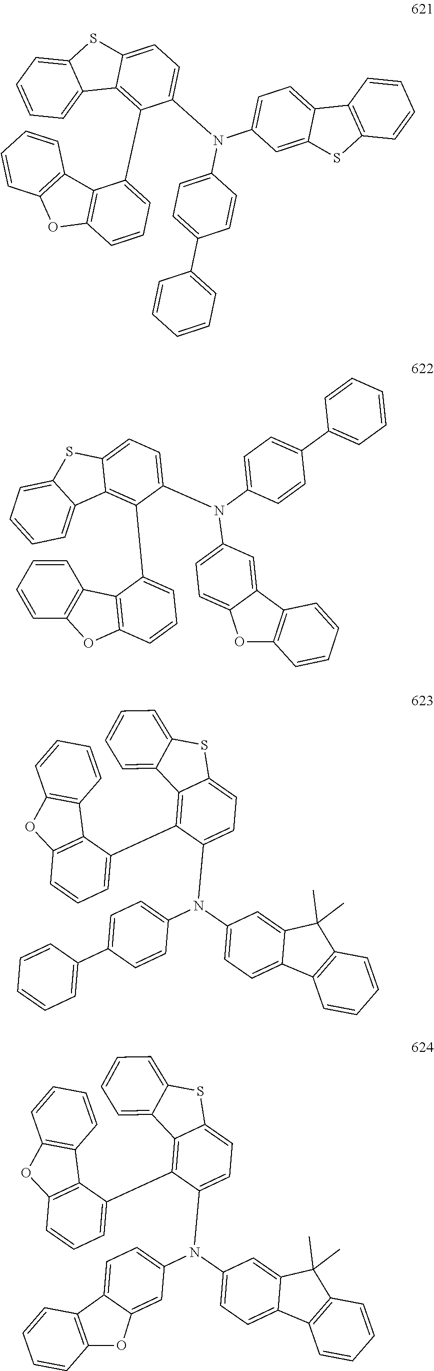 [SMILES: c1ccc(-c2ccc(N(c3ccc4c(c3)sc3ccccc34)c3ccc4sc5ccccc5c4c3-c3cccc4oc5ccccc5c34)cc2)cc1 ; c1ccc(-c2ccc(N(c3ccc4oc5ccccc5c4c3)c3ccc4sc5ccccc5c4c3-c3cccc4oc5ccccc5c34)cc2)cc1 ; CC1(C)c2ccccc2-c2ccc(N(c3ccc(-c4ccccc4)cc3)c3ccc4sc5ccccc5c4c3-c3cccc4oc5ccccc5c34)cc21 ; CC1(C)c2ccccc2-c2ccc(N(c3ccc4c(c3)oc3ccccc34)c3ccc4sc5ccccc5c4c3-c3cccc4oc5ccccc5c34)cc21]

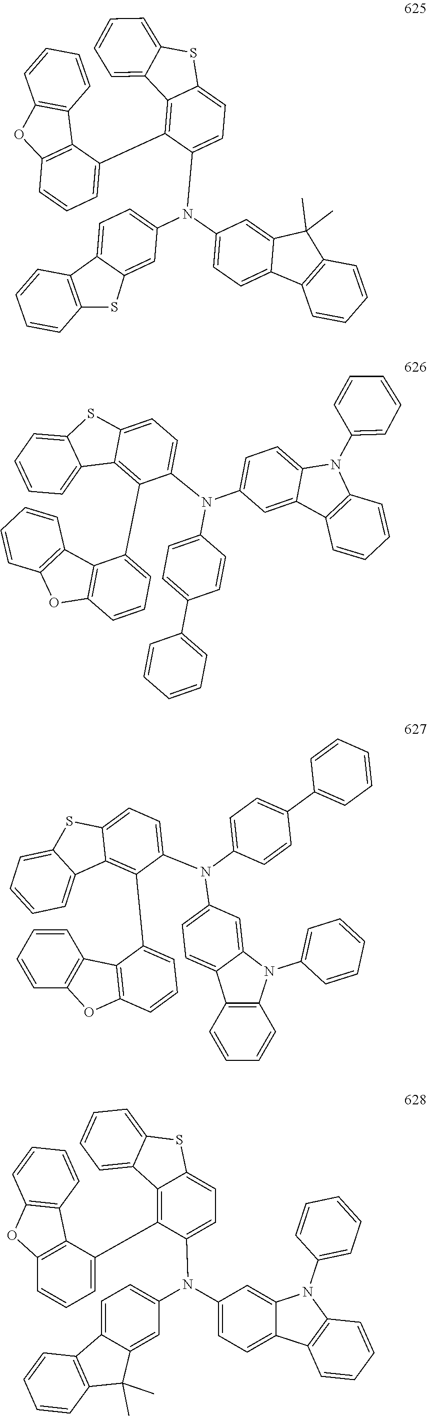 [SMILES: CC1(C)c2ccccc2-c2ccc(N(c3ccc4c(c3)sc3ccccc34)c3ccc4sc5ccccc5c4c3-c3cccc4oc5ccccc5c34)cc21 ; c1ccc(-c2ccc(N(c3ccc4c(c3)c3ccccc3n4-c3ccccc3)c3ccc4sc5ccccc5c4c3-c3cccc4oc5ccccc5c34)cc2)cc1 ; c1ccc(-c2ccc(N(c3ccc4c5ccccc5n(-c5ccccc5)c4c3)c3ccc4sc5ccccc5c4c3-c3cccc4oc5ccccc5c34)cc2)cc1 ; CC1(C)c2ccccc2-c2ccc(N(c3ccc4c5ccccc5n(-c5ccccc5)c4c3)c3ccc4sc5ccccc5c4c3-c3cccc4oc5ccccc5c34)cc21]

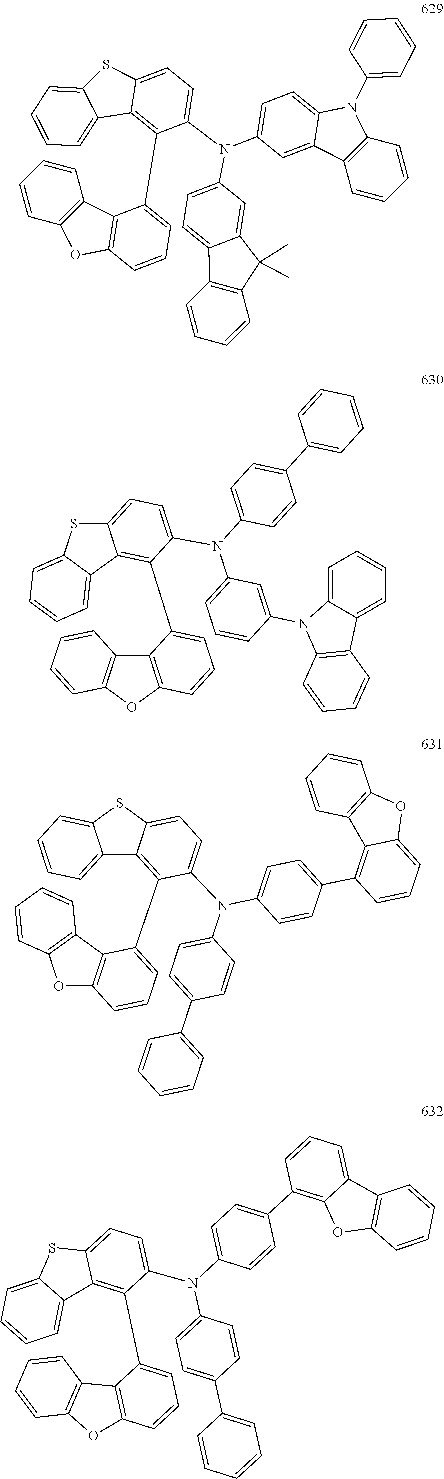

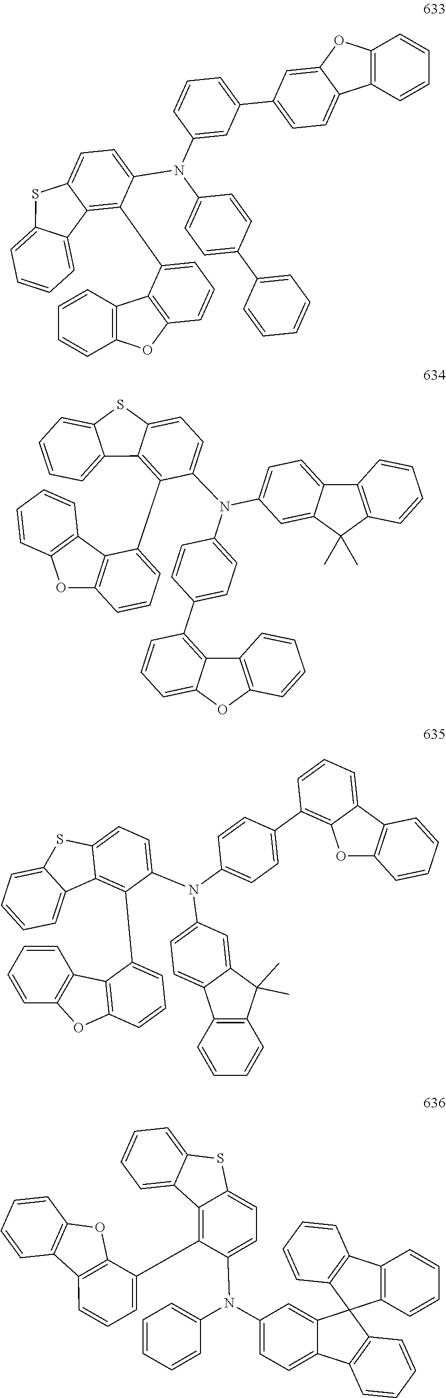 [SMILES: c1ccc(-c2ccc(N(c3cccc(-c4ccc5c(c4)oc4ccccc45)c3)c3ccc4sc5ccccc5c4c3-c3cccc4oc5ccccc5c34)cc2)cc1 ; CC1(C)c2ccccc2-c2ccc(N(c3ccc(-c4cccc5oc6ccccc6c45)cc3)c3ccc4sc5ccccc5c4c3-c3cccc4oc5ccccc5c34)cc21 ; CC1(C)c2ccccc2-c2ccc(N(c3ccc(-c4cccc5c4oc4ccccc45)cc3)c3ccc4sc5ccccc5c4c3-c3cccc4oc5ccccc5c34)cc21 ; c1ccc(N(c2ccc3c(c2)C2(c4ccccc4-c4ccccc42)c2ccccc2-3)c2ccc3sc4ccccc4c3c2-c2cccc3c2oc2ccccc23)cc1]

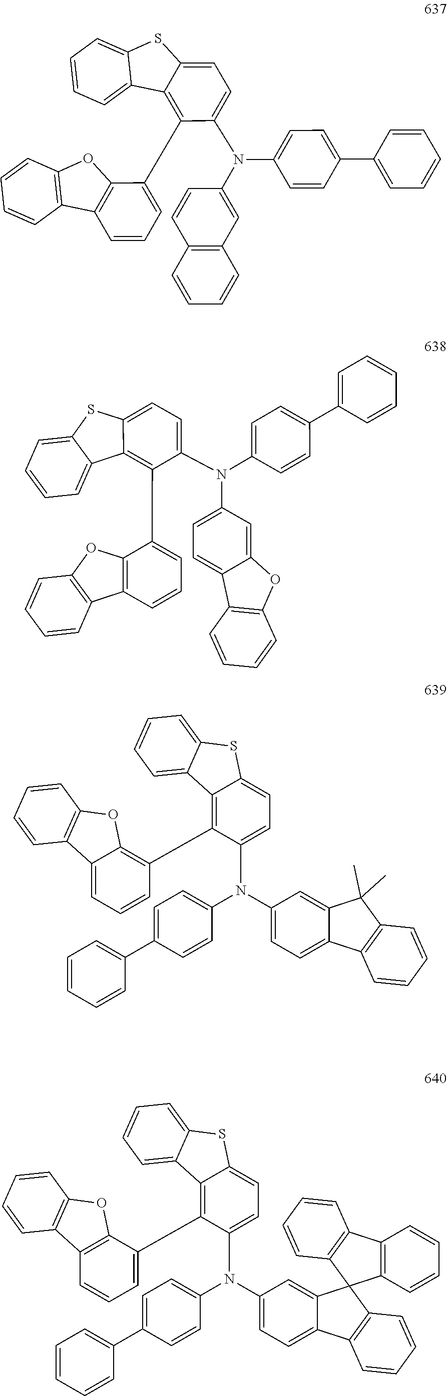 [SMILES: c1ccc(-c2ccc(N(c3ccc4ccccc4c3)c3ccc4sc5ccccc5c4c3-c3cccc4c3oc3ccccc34)cc2)cc1 ; c1ccc(-c2ccc(N(c3ccc4c(c3)oc3ccccc34)c3ccc4sc5ccccc5c4c3-c3cccc4c3oc3ccccc34)cc2)cc1 ; CC1(C)c2ccccc2-c2ccc(N(c3ccc(-c4ccccc4)cc3)c3ccc4sc5ccccc5c4c3-c3cccc4c3oc3ccccc34)cc21 ; c1ccc(-c2ccc(N(c3ccc4c(c3)C3(c5ccccc5-c5ccccc53)c3ccccc3-4)c3ccc4sc5ccccc5c4c3-c3cccc4c3oc3ccccc34)cc2)cc1]

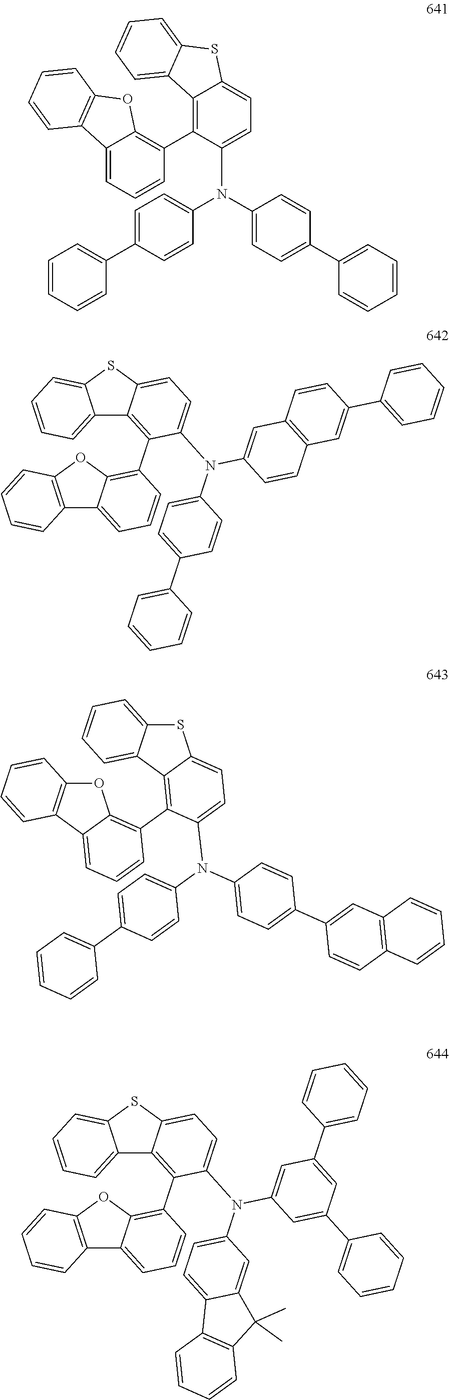 [SMILES: c1ccc(-c2ccc(N(c3ccc(-c4ccccc4)cc3)c3ccc4sc5ccccc5c4c3-c3cccc4c3oc3ccccc34)cc2)cc1 ; c1ccc(-c2ccc(N(c3ccc4cc(-c5ccccc5)ccc4c3)c3ccc4sc5ccccc5c4c3-c3cccc4c3oc3ccccc34)cc2)cc1 ; c1ccc(-c2ccc(N(c3ccc(-c4ccc5ccccc5c4)cc3)c3ccc4sc5ccccc5c4c3-c3cccc4c3oc3ccccc34)cc2)cc1 ; CC1(C)c2ccccc2-c2ccc(N(c3cc(-c4ccccc4)cc(-c4ccccc4)c3)c3ccc4sc5ccccc5c4c3-c3cccc4c3oc3ccccc34)cc21]

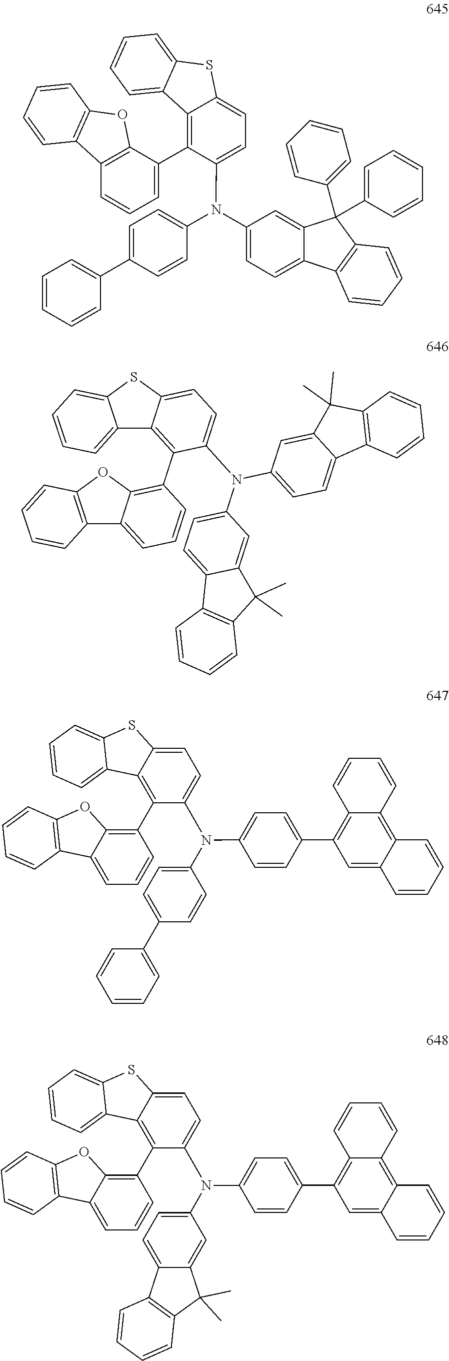 [SMILES: c1ccc(-c2ccc(N(c3ccc4c(c3)C(c3ccccc3)(c3ccccc3)c3ccccc3-4)c3ccc4sc5ccccc5c4c3-c3cccc4c3oc3ccccc34)cc2)cc1 ; CC1(C)c2ccccc2-c2ccc(N(c3ccc4c(c3)C(C)(C)c3ccccc3-4)c3ccc4sc5ccccc5c4c3-c3cccc4c3oc3ccccc34)cc21 ; c1ccc(-c2ccc(N(c3ccc(-c4cc5ccccc5c5ccccc45)cc3)c3ccc4sc5ccccc5c4c3-c3cccc4c3oc3ccccc34)cc2)cc1 ; CC1(C)c2ccccc2-c2ccc(N(c3ccc(-c4cc5ccccc5c5ccccc45)cc3)c3ccc4sc5ccccc5c4c3-c3cccc4c3oc3ccccc34)cc21]

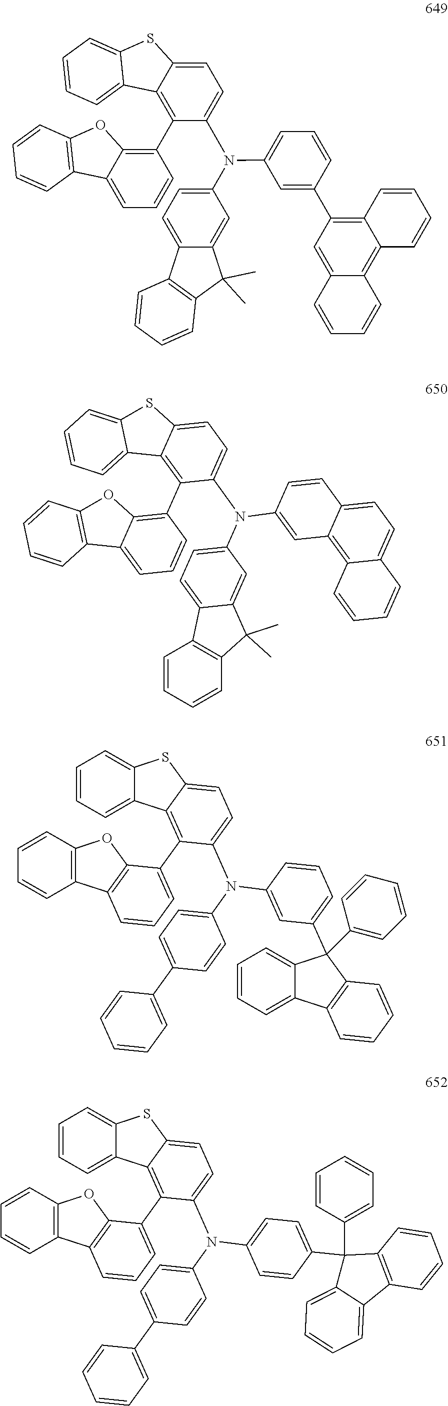 [SMILES: CC1(C)c2ccccc2-c2ccc(N(c3cccc(-c4cc5ccccc5c5ccccc45)c3)c3ccc4sc5ccccc5c4c3-c3cccc4c3oc3ccccc34)cc21 ; CC1(C)c2ccccc2-c2ccc(N(c3ccc4ccc5ccccc5c4c3)c3ccc4sc5ccccc5c4c3-c3cccc4c3oc3ccccc34)cc21 ; c1ccc(-c2ccc(N(c3cccc(C4(c5ccccc5)c5ccccc5-c5ccccc54)c3)c3ccc4sc5ccccc5c4c3-c3cccc4c3oc3ccccc34)cc2)cc1 ; c1ccc(-c2ccc(N(c3ccc(C4(c5ccccc5)c5ccccc5-c5ccccc54)cc3)c3ccc4sc5ccccc5c4c3-c3cccc4c3oc3ccccc34)cc2)cc1]

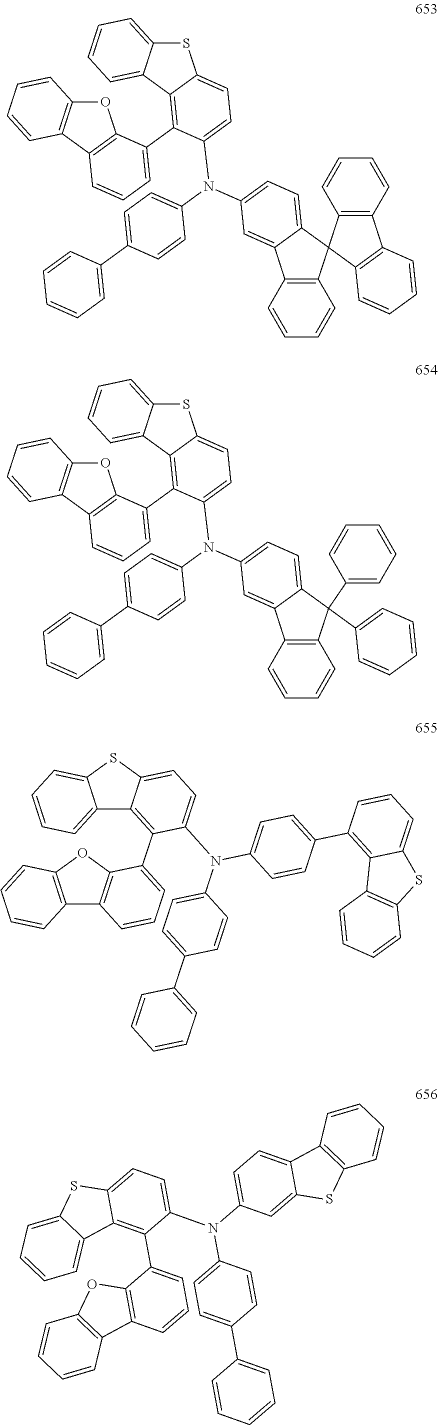 [SMILES: c1ccc(-c2ccc(N(c3ccc4c(c3)-c3ccccc3C43c4ccccc4-c4ccccc43)c3ccc4sc5ccccc5c4c3-c3cccc4c3oc3ccccc34)cc2)cc1 ; c1ccc(-c2ccc(N(c3ccc4c(c3)-c3ccccc3C4(c3ccccc3)c3ccccc3)c3ccc4sc5ccccc5c4c3-c3cccc4c3oc3ccccc34)cc2)cc1 ; c1ccc(-c2ccc(N(c3ccc(-c4cccc5sc6ccccc6c45)cc3)c3ccc4sc5ccccc5c4c3-c3cccc4c3oc3ccccc34)cc2)cc1 ; c1ccc(-c2ccc(N(c3ccc4c(c3)sc3ccccc34)c3ccc4sc5ccccc5c4c3-c3cccc4c3oc3ccccc34)cc2)cc1]

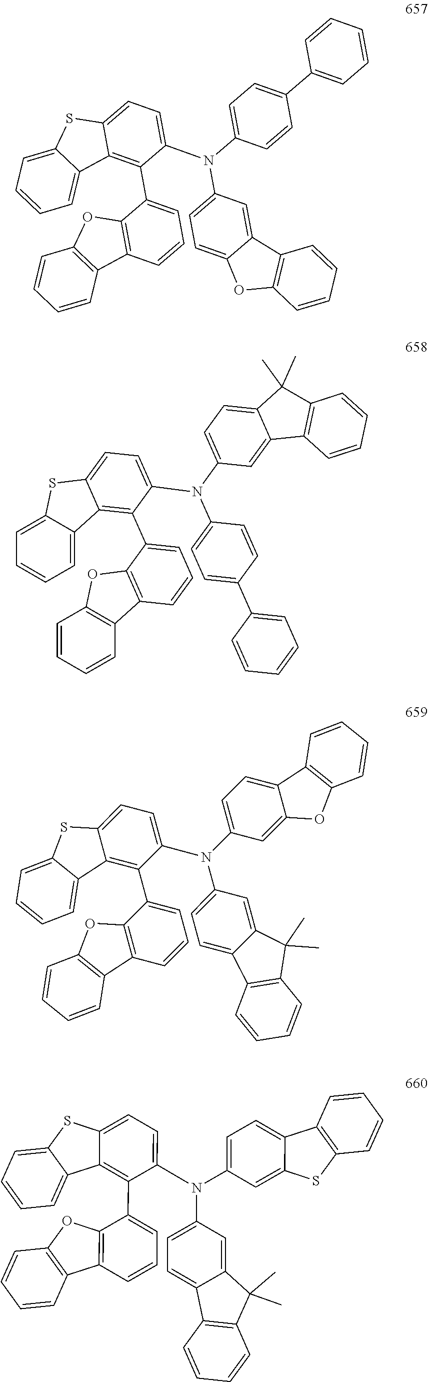

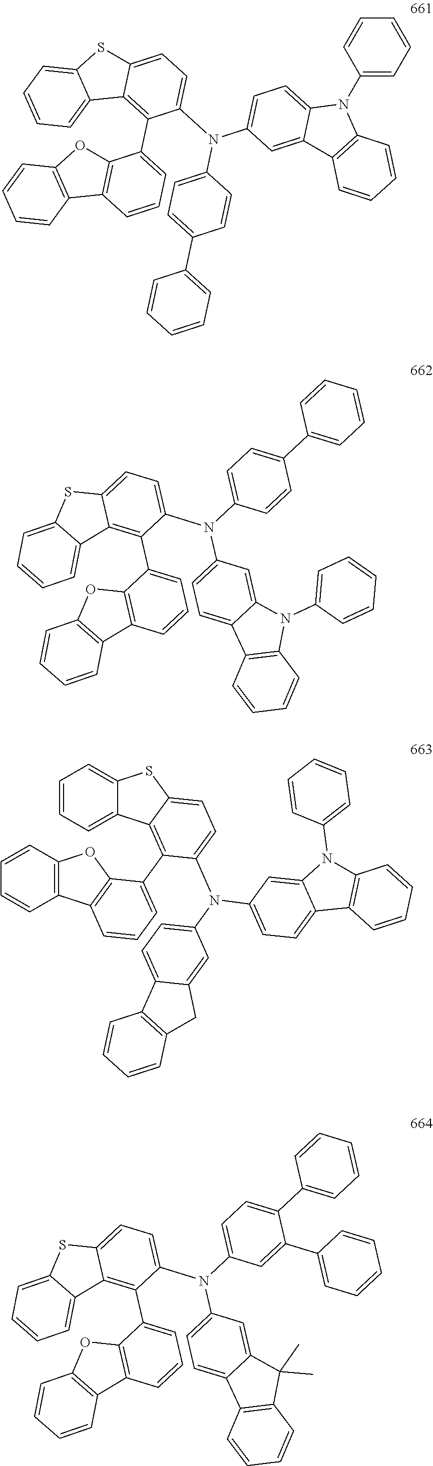 [SMILES: c1ccc(-c2ccc(N(c3ccc4c(c3)c3ccccc3n4-c3ccccc3)c3ccc4sc5ccccc5c4c3-c3cccc4c3oc3ccccc34)cc2)cc1 ; c1ccc(-c2ccc(N(c3ccc4c5ccccc5n(-c5ccccc5)c4c3)c3ccc4sc5ccccc5c4c3-c3cccc4c3oc3ccccc34)cc2)cc1 ; c1ccc(-n2c3ccccc3c3ccc(N(c4ccc5c(c4)Cc4ccccc4-5)c4ccc5sc6ccccc6c5c4-c4cccc5c4oc4ccccc45)cc32)cc1 ; CC1(C)c2ccccc2-c2ccc(N(c3ccc(-c4ccccc4)c(-c4ccccc4)c3)c3ccc4sc5ccccc5c4c3-c3cccc4c3oc3ccccc34)cc21]

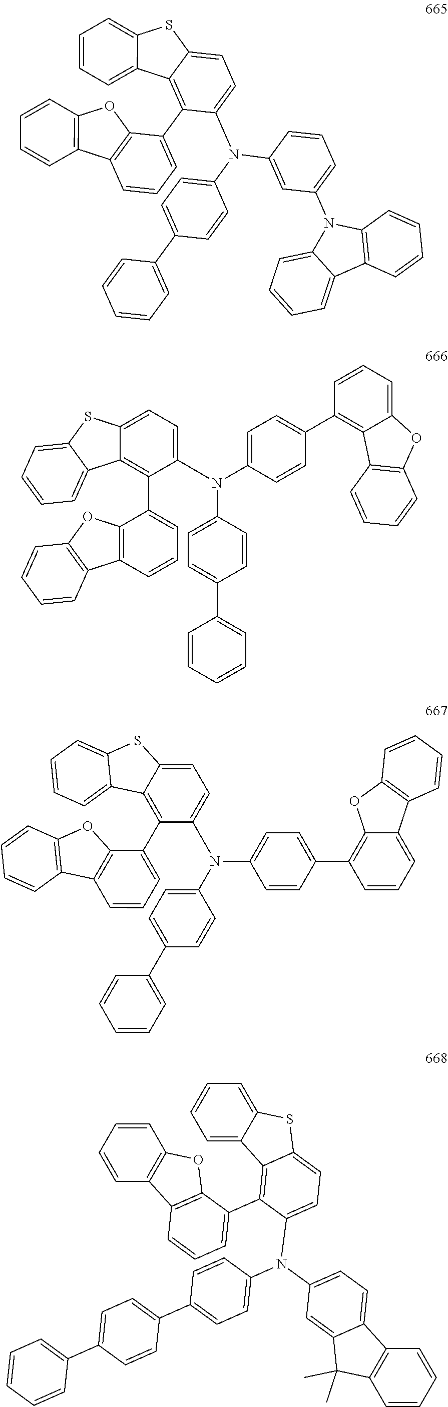 [SMILES: c1ccc(-c2ccc(N(c3cccc(-n4c5ccccc5c5ccccc54)c3)c3ccc4sc5ccccc5c4c3-c3cccc4c3oc3ccccc34)cc2)cc1 ; c1ccc(-c2ccc(N(c3ccc(-c4cccc5oc6ccccc6c45)cc3)c3ccc4sc5ccccc5c4c3-c3cccc4c3oc3ccccc34)cc2)cc1 ; c1ccc(-c2ccc(N(c3ccc(-c4cccc5c4oc4ccccc45)cc3)c3ccc4sc5ccccc5c4c3-c3cccc4c3oc3ccccc34)cc2)cc1 ; CC1(C)c2ccccc2-c2ccc(N(c3ccc(-c4ccc(-c5ccccc5)cc4)cc3)c3ccc4sc5ccccc5c4c3-c3cccc4c3oc3ccccc34)cc21]

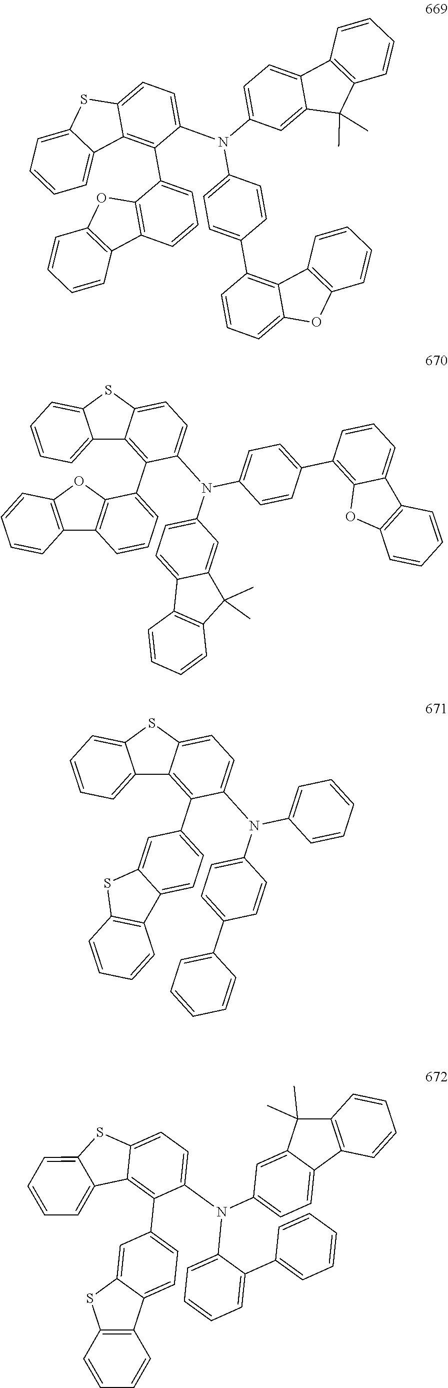 [SMILES: CC1(C)c2ccccc2-c2ccc(N(c3ccc(-c4cccc5oc6ccccc6c45)cc3)c3ccc4sc5ccccc5c4c3-c3cccc4c3oc3ccccc34)cc21 ; CC1(C)c2ccccc2-c2ccc(N(c3ccc(-c4cccc5c4oc4ccccc45)cc3)c3ccc4sc5ccccc5c4c3-c3cccc4c3oc3ccccc34)cc21 ; c1ccc(-c2ccc(N(c3ccccc3)c3ccc4sc5ccccc5c4c3-c3ccc4c(c3)sc3ccccc34)cc2)cc1 ; CC1(C)c2ccccc2-c2ccc(N(c3ccccc3-c3ccccc3)c3ccc4sc5ccccc5c4c3-c3ccc4c(c3)sc3ccccc34)cc21]

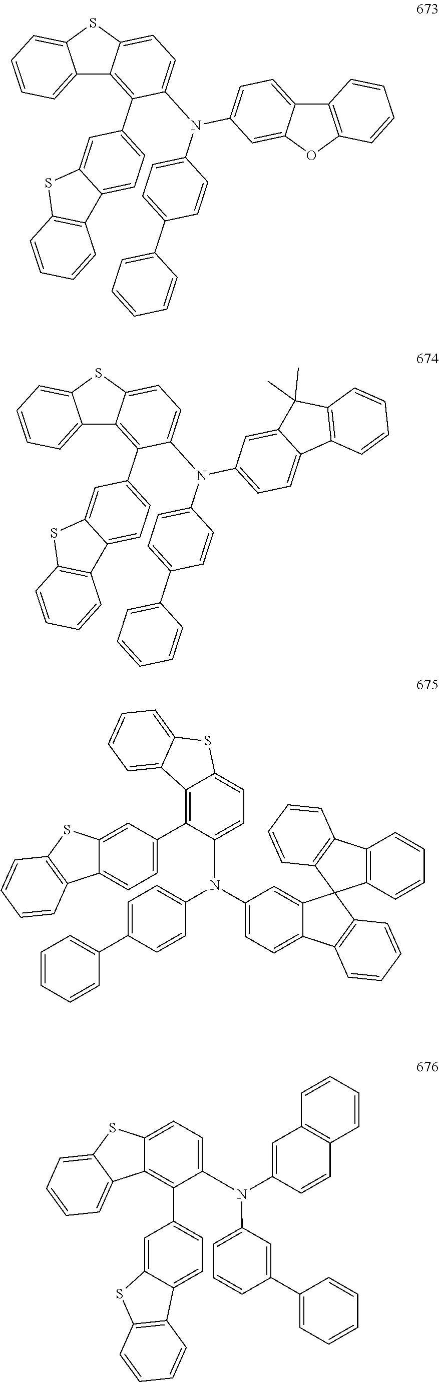

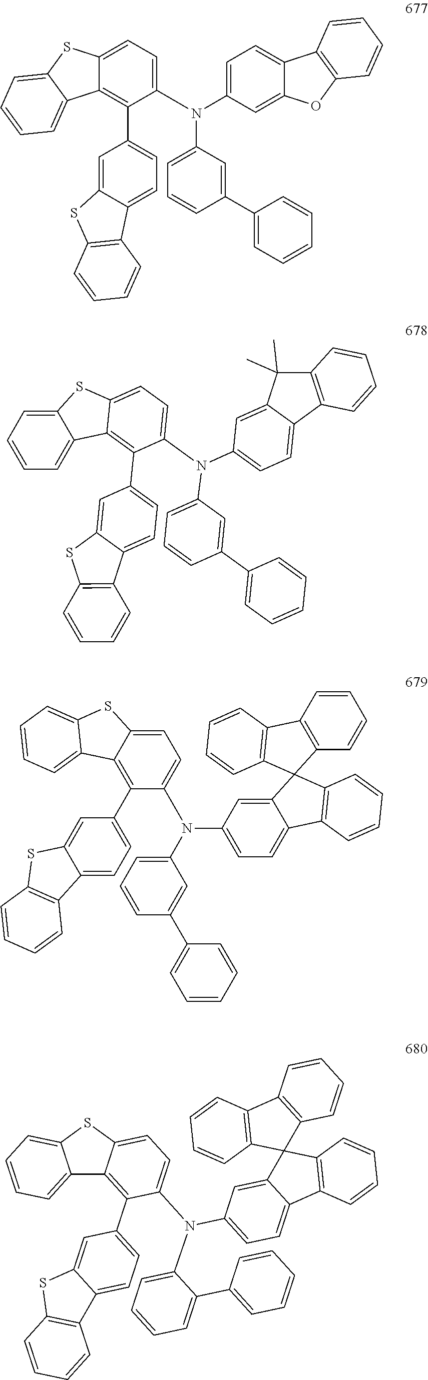 [SMILES: c1ccc(-c2cccc(N(c3ccc4c(c3)oc3ccccc34)c3ccc4sc5ccccc5c4c3-c3ccc4c(c3)sc3ccccc34)c2)cc1 ; CC1(C)c2ccccc2-c2ccc(N(c3cccc(-c4ccccc4)c3)c3ccc4sc5ccccc5c4c3-c3ccc4c(c3)sc3ccccc34)cc21 ; c1ccc(-c2cccc(N(c3ccc4c(c3)C3(c5ccccc5-c5ccccc53)c3ccccc3-4)c3ccc4sc5ccccc5c4c3-c3ccc4c(c3)sc3ccccc34)c2)cc1 ; c1ccc(-c2ccccc2N(c2ccc3c(c2)C2(c4ccccc4-c4ccccc42)c2ccccc2-3)c2ccc3sc4ccccc4c3c2-c2ccc3c(c2)sc2ccccc23)cc1]

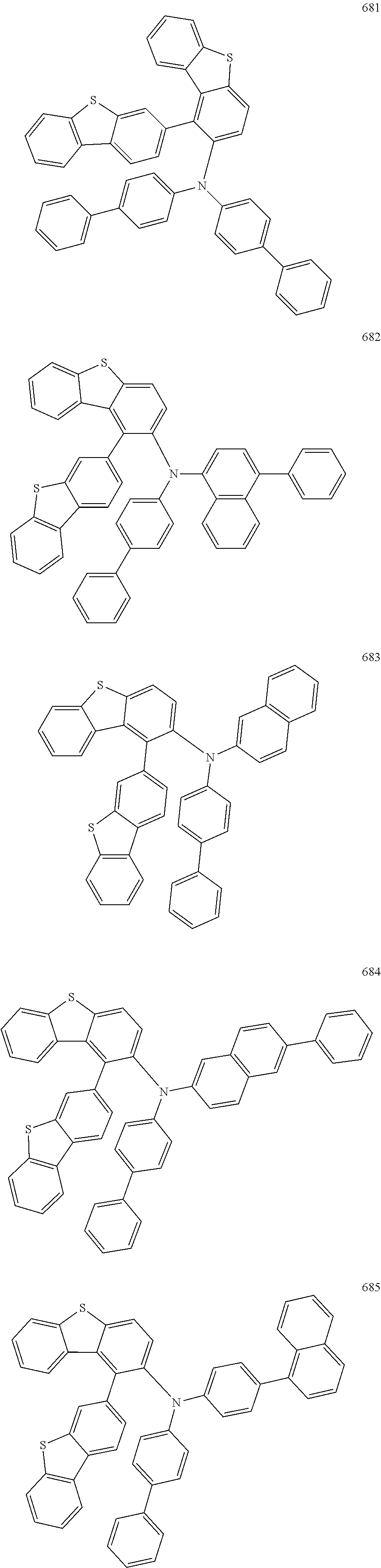 [SMILES: c1ccc(-c2ccc(N(c3ccc(-c4ccccc4)cc3)c3ccc4sc5ccccc5c4c3-c3ccc4c(c3)sc3ccccc34)cc2)cc1 ; c1ccc(-c2ccc(N(c3ccc4sc5ccccc5c4c3-c3ccc4c(c3)sc3ccccc34)c3ccc(-c4ccccc4)c4ccccc34)cc2)cc1 ; c1ccc(-c2ccc(N(c3ccc4ccccc4c3)c3ccc4sc5ccccc5c4c3-c3ccc4c(c3)sc3ccccc34)cc2)cc1 ; c1ccc(-c2ccc(N(c3ccc4cc(-c5ccccc5)ccc4c3)c3ccc4sc5ccccc5c4c3-c3ccc4c(c3)sc3ccccc34)cc2)cc1 ; c1ccc(-c2ccc(N(c3ccc(-c4cccc5ccccc45)cc3)c3ccc4sc5ccccc5c4c3-c3ccc4c(c3)sc3ccccc34)cc2)cc1]

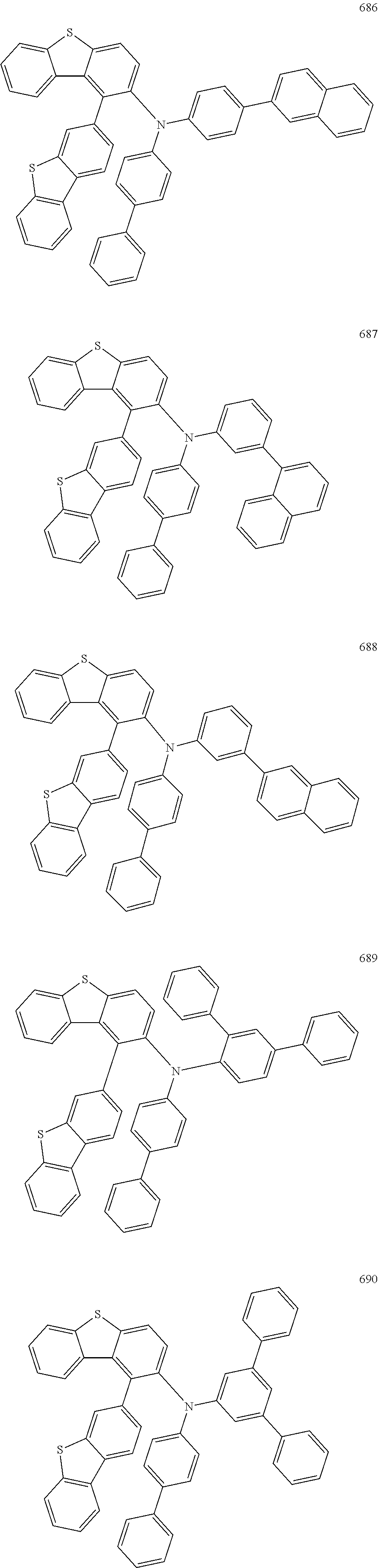 [SMILES: c1ccc(-c2ccc(N(c3ccc(-c4ccc5ccccc5c4)cc3)c3ccc4sc5ccccc5c4c3-c3ccc4c(c3)sc3ccccc34)cc2)cc1 ; c1ccc(-c2ccc(N(c3cccc(-c4cccc5ccccc45)c3)c3ccc4sc5ccccc5c4c3-c3ccc4c(c3)sc3ccccc34)cc2)cc1 ; c1ccc(-c2ccc(N(c3cccc(-c4ccc5ccccc5c4)c3)c3ccc4sc5ccccc5c4c3-c3ccc4c(c3)sc3ccccc34)cc2)cc1 ; c1ccc(-c2ccc(N(c3ccc(-c4ccccc4)cc3-c3ccccc3)c3ccc4sc5ccccc5c4c3-c3ccc4c(c3)sc3ccccc34)cc2)cc1 ; c1ccc(-c2ccc(N(c3cc(-c4ccccc4)cc(-c4ccccc4)c3)c3ccc4sc5ccccc5c4c3-c3ccc4c(c3)sc3ccccc34)cc2)cc1]

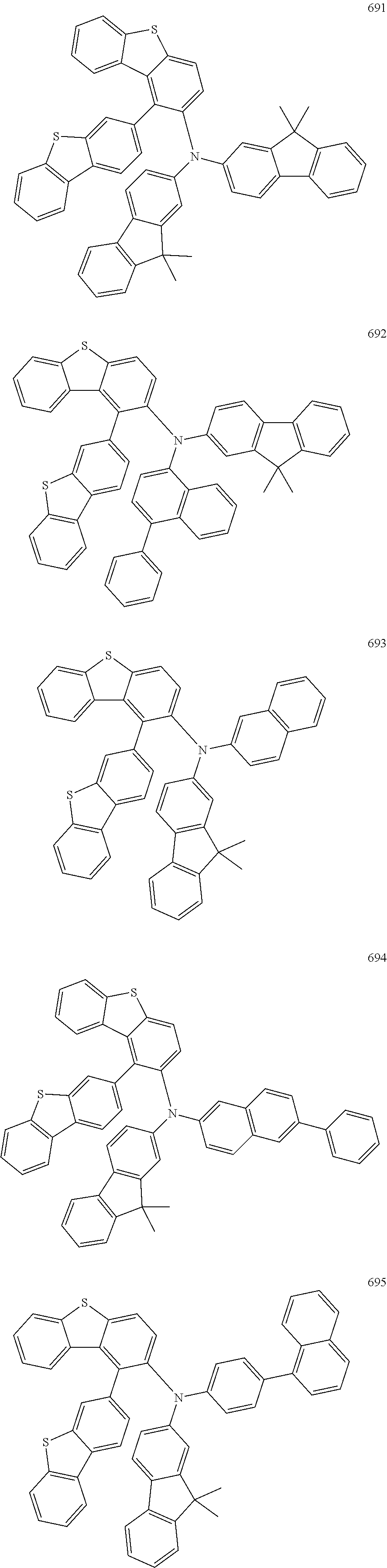 [SMILES: CC1(C)c2ccccc2-c2ccc(N(c3ccc4c(c3)C(C)(C)c3ccccc3-4)c3ccc4sc5ccccc5c4c3-c3ccc4c(c3)sc3ccccc34)cc21 ; CC1(C)c2ccccc2-c2ccc(N(c3ccc4sc5ccccc5c4c3-c3ccc4c(c3)sc3ccccc34)c3ccc(-c4ccccc4)c4ccccc34)cc21 ; CC1(C)c2ccccc2-c2ccc(N(c3ccc4ccccc4c3)c3ccc4sc5ccccc5c4c3-c3ccc4c(c3)sc3ccccc34)cc21 ; CC1(C)c2ccccc2-c2ccc(N(c3ccc4cc(-c5ccccc5)ccc4c3)c3ccc4sc5ccccc5c4c3-c3ccc4c(c3)sc3ccccc34)cc21 ; CC1(C)c2ccccc2-c2ccc(N(c3ccc(-c4cccc5ccccc45)cc3)c3ccc4sc5ccccc5c4c3-c3ccc4c(c3)sc3ccccc34)cc21]

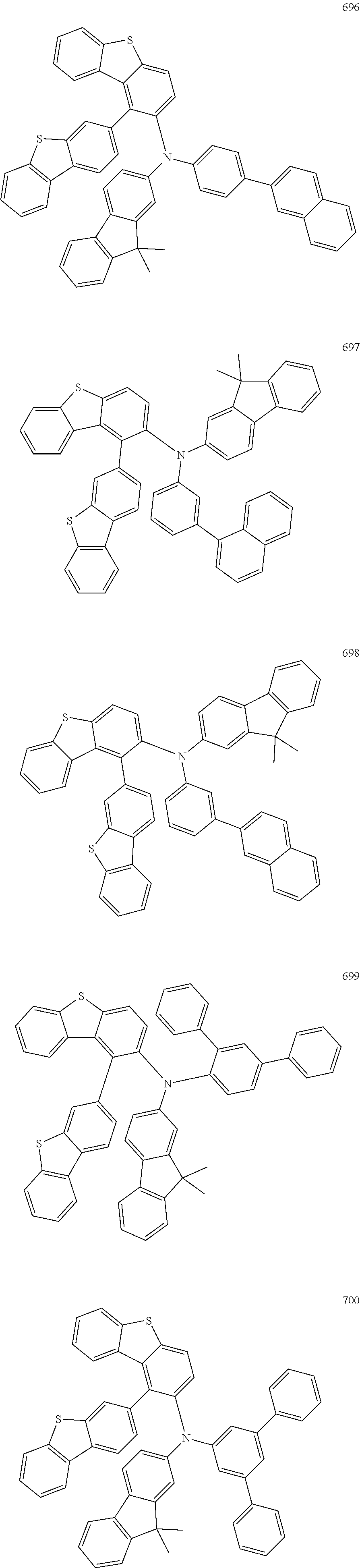 [SMILES: CC1(C)c2ccccc2-c2ccc(N(c3ccc(-c4ccc5ccccc5c4)cc3)c3ccc4sc5ccccc5c4c3-c3ccc4c(c3)sc3ccccc34)cc21 ; CC1(C)c2ccccc2-c2ccc(N(c3cccc(-c4cccc5ccccc45)c3)c3ccc4sc5ccccc5c4c3-c3ccc4c(c3)sc3ccccc34)cc21 ; CC1(C)c2ccccc2-c2ccc(N(c3cccc(-c4ccc5ccccc5c4)c3)c3ccc4sc5ccccc5c4c3-c3ccc4c(c3)sc3ccccc34)cc21 ; CC1(C)c2ccccc2-c2ccc(N(c3ccc(-c4ccccc4)cc3-c3ccccc3)c3ccc4sc5ccccc5c4c3-c3ccc4c(c3)sc3ccccc34)cc21 ; CC1(C)c2ccccc2-c2ccc(N(c3cc(-c4ccccc4)cc(-c4ccccc4)c3)c3ccc4sc5ccccc5c4c3-c3ccc4c(c3)sc3ccccc34)cc21]

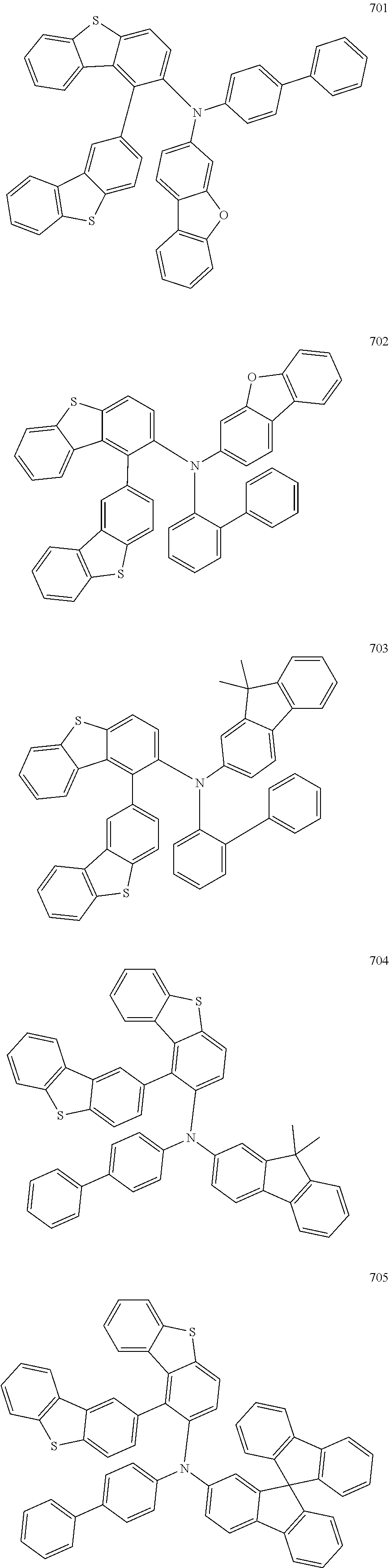 [SMILES: c1ccc(-c2ccc(N(c3ccc4c(c3)oc3ccccc34)c3ccc4sc5ccccc5c4c3-c3ccc4sc5ccccc5c4c3)cc2)cc1 ; c1ccc(-c2ccccc2N(c2ccc3c(c2)oc2ccccc23)c2ccc3sc4ccccc4c3c2-c2ccc3sc4ccccc4c3c2)cc1 ; CC1(C)c2ccccc2-c2ccc(N(c3ccccc3-c3ccccc3)c3ccc4sc5ccccc5c4c3-c3ccc4sc5ccccc5c4c3)cc21 ; CC1(C)c2ccccc2-c2ccc(N(c3ccc(-c4ccccc4)cc3)c3ccc4sc5ccccc5c4c3-c3ccc4sc5ccccc5c4c3)cc21 ; c1ccc(-c2ccc(N(c3ccc4c(c3)C3(c5ccccc5-c5ccccc53)c3ccccc3-4)c3ccc4sc5ccccc5c4c3-c3ccc4sc5ccccc5c4c3)cc2)cc1]

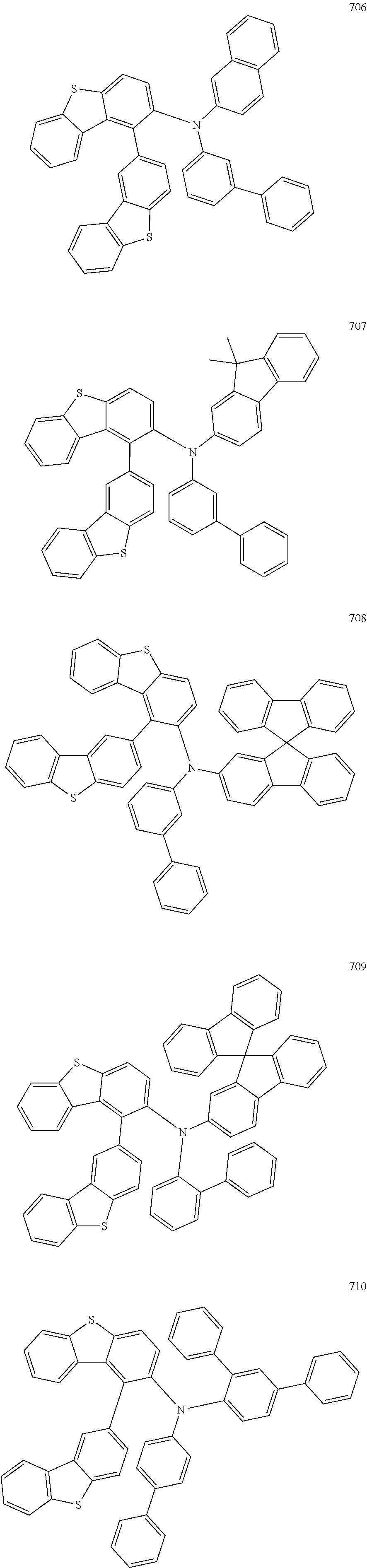 [SMILES: c1ccc(-c2cccc(N(c3ccc4ccccc4c3)c3ccc4sc5ccccc5c4c3-c3ccc4sc5ccccc5c4c3)c2)cc1 ; CC1(C)c2ccccc2-c2ccc(N(c3cccc(-c4ccccc4)c3)c3ccc4sc5ccccc5c4c3-c3ccc4sc5ccccc5c4c3)cc21 ; c1ccc(-c2cccc(N(c3ccc4c(c3)C3(c5ccccc5-c5ccccc53)c3ccccc3-4)c3ccc4sc5ccccc5c4c3-c3ccc4sc5ccccc5c4c3)c2)cc1 ; c1ccc(-c2ccccc2N(c2ccc3c(c2)C2(c4ccccc4-c4ccccc42)c2ccccc2-3)c2ccc3sc4ccccc4c3c2-c2ccc3sc4ccccc4c3c2)cc1 ; c1ccc(-c2ccc(N(c3ccc(-c4ccccc4)cc3-c3ccccc3)c3ccc4sc5ccccc5c4c3-c3ccc4sc5ccccc5c4c3)cc2)cc1]

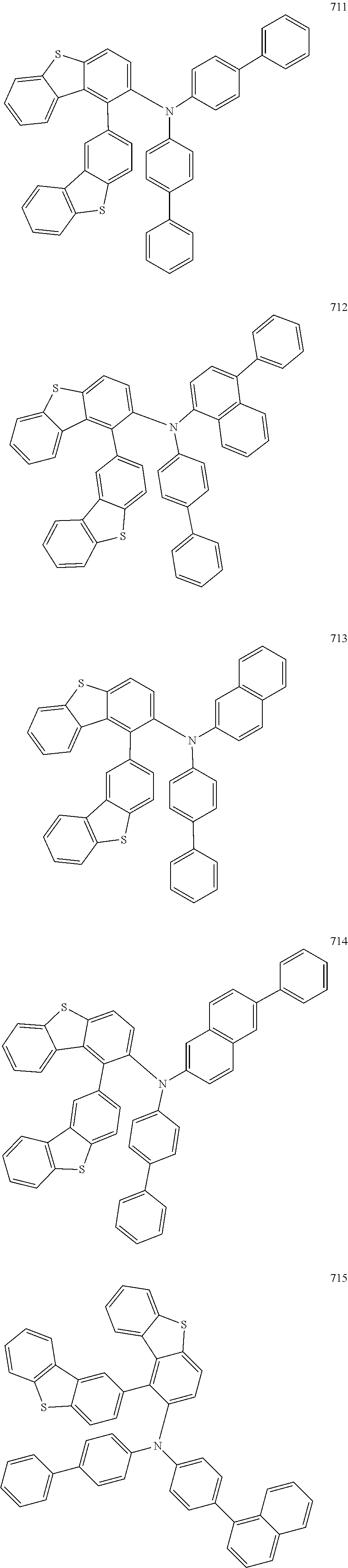 [SMILES: c1ccc(-c2ccc(N(c3ccc(-c4ccccc4)cc3)c3ccc4sc5ccccc5c4c3-c3ccc4sc5ccccc5c4c3)cc2)cc1 ; c1ccc(-c2ccc(N(c3ccc4sc5ccccc5c4c3-c3ccc4sc5ccccc5c4c3)c3ccc(-c4ccccc4)c4ccccc34)cc2)cc1 ; c1ccc(-c2ccc(N(c3ccc4ccccc4c3)c3ccc4sc5ccccc5c4c3-c3ccc4sc5ccccc5c4c3)cc2)cc1 ; c1ccc(-c2ccc(N(c3ccc4cc(-c5ccccc5)ccc4c3)c3ccc4sc5ccccc5c4c3-c3ccc4sc5ccccc5c4c3)cc2)cc1 ; c1ccc(-c2ccc(N(c3ccc(-c4cccc5ccccc45)cc3)c3ccc4sc5ccccc5c4c3-c3ccc4sc5ccccc5c4c3)cc2)cc1]

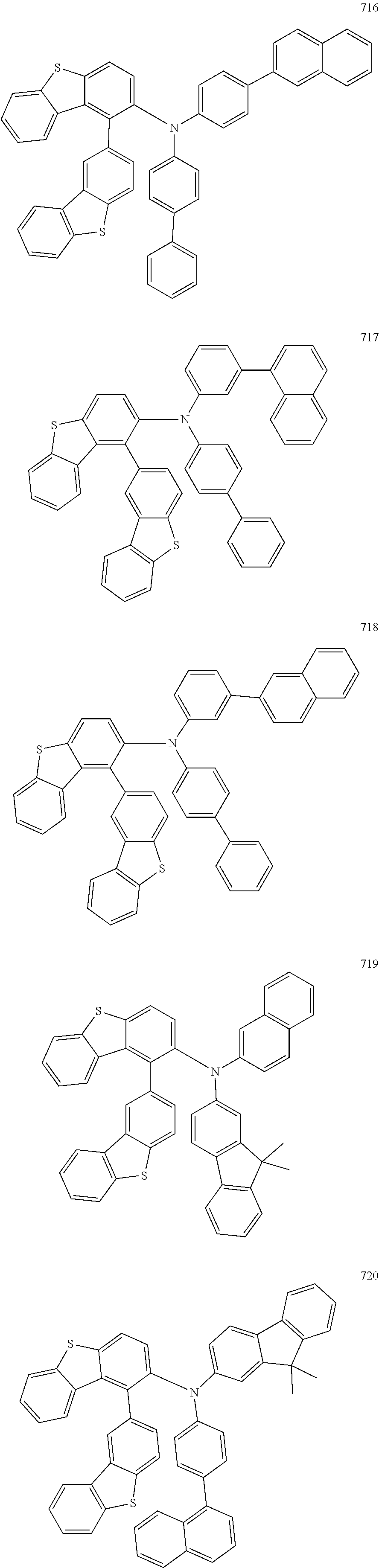 [SMILES: c1ccc(-c2ccc(N(c3ccc(-c4ccc5ccccc5c4)cc3)c3ccc4sc5ccccc5c4c3-c3ccc4sc5ccccc5c4c3)cc2)cc1 ; c1ccc(-c2ccc(N(c3cccc(-c4cccc5ccccc45)c3)c3ccc4sc5ccccc5c4c3-c3ccc4sc5ccccc5c4c3)cc2)cc1 ; c1ccc(-c2ccc(N(c3cccc(-c4ccc5ccccc5c4)c3)c3ccc4sc5ccccc5c4c3-c3ccc4sc5ccccc5c4c3)cc2)cc1 ; CC1(C)c2ccccc2-c2ccc(N(c3ccc4ccccc4c3)c3ccc4sc5ccccc5c4c3-c3ccc4sc5ccccc5c4c3)cc21 ; CC1(C)c2ccccc2-c2ccc(N(c3ccc(-c4cccc5ccccc45)cc3)c3ccc4sc5ccccc5c4c3-c3ccc4sc5ccccc5c4c3)cc21]

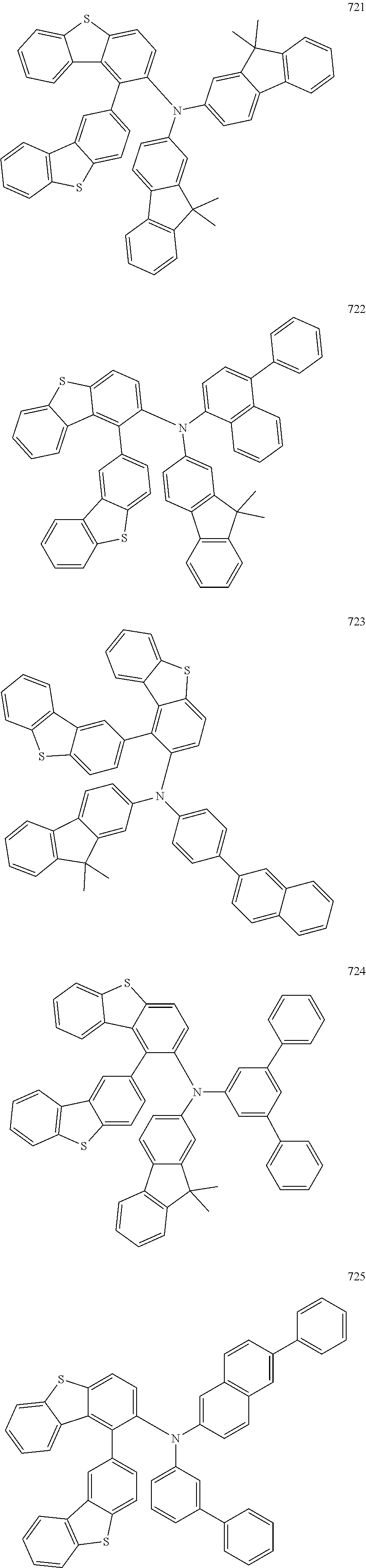 [SMILES: CC1(C)c2ccccc2-c2ccc(N(c3ccc4c(c3)C(C)(C)c3ccccc3-4)c3ccc4sc5ccccc5c4c3-c3ccc4sc5ccccc5c4c3)cc21 ; CC1(C)c2ccccc2-c2ccc(N(c3ccc4sc5ccccc5c4c3-c3ccc4sc5ccccc5c4c3)c3ccc(-c4ccccc4)c4ccccc34)cc21 ; CC1(C)c2ccccc2-c2ccc(N(c3ccc(-c4ccc5ccccc5c4)cc3)c3ccc4sc5ccccc5c4c3-c3ccc4sc5ccccc5c4c3)cc21 ; CC1(C)c2ccccc2-c2ccc(N(c3cc(-c4ccccc4)cc(-c4ccccc4)c3)c3ccc4sc5ccccc5c4c3-c3ccc4sc5ccccc5c4c3)cc21 ; c1ccc(-c2cccc(N(c3ccc4cc(-c5ccccc5)ccc4c3)c3ccc4sc5ccccc5c4c3-c3ccc4sc5ccccc5c4c3)c2)cc1]

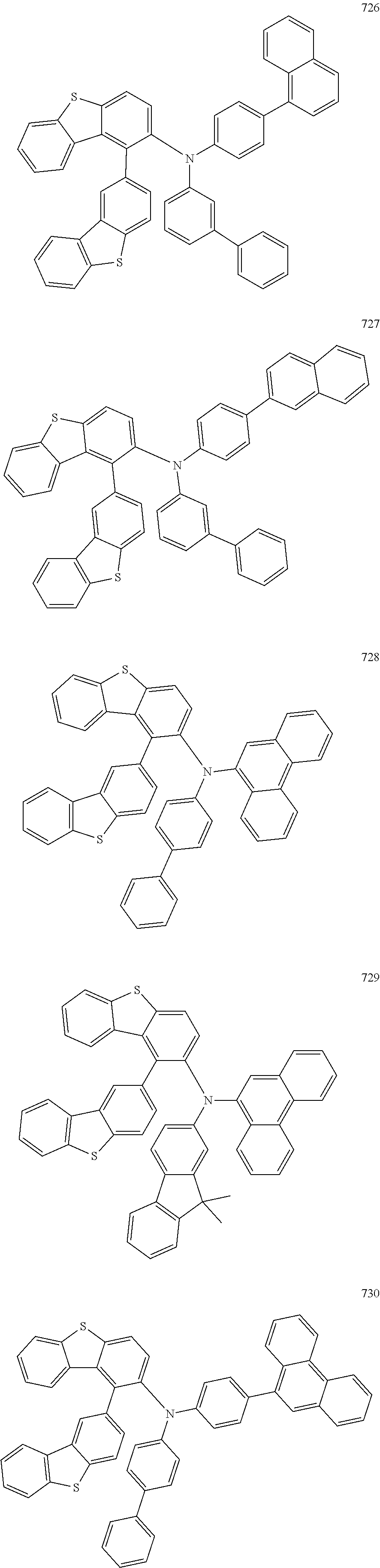 [SMILES: c1ccc(-c2cccc(N(c3ccc(-c4cccc5ccccc45)cc3)c3ccc4sc5ccccc5c4c3-c3ccc4sc5ccccc5c4c3)c2)cc1 ; c1ccc(-c2cccc(N(c3ccc(-c4ccc5ccccc5c4)cc3)c3ccc4sc5ccccc5c4c3-c3ccc4sc5ccccc5c4c3)c2)cc1 ; c1ccc(-c2ccc(N(c3ccc4sc5ccccc5c4c3-c3ccc4sc5ccccc5c4c3)c3cc4ccccc4c4ccccc34)cc2)cc1 ; CC1(C)c2ccccc2-c2ccc(N(c3ccc4sc5ccccc5c4c3-c3ccc4sc5ccccc5c4c3)c3cc4ccccc4c4ccccc34)cc21 ; c1ccc(-c2ccc(N(c3ccc(-c4cc5ccccc5c5ccccc45)cc3)c3ccc4sc5ccccc5c4c3-c3ccc4sc5ccccc5c4c3)cc2)cc1]

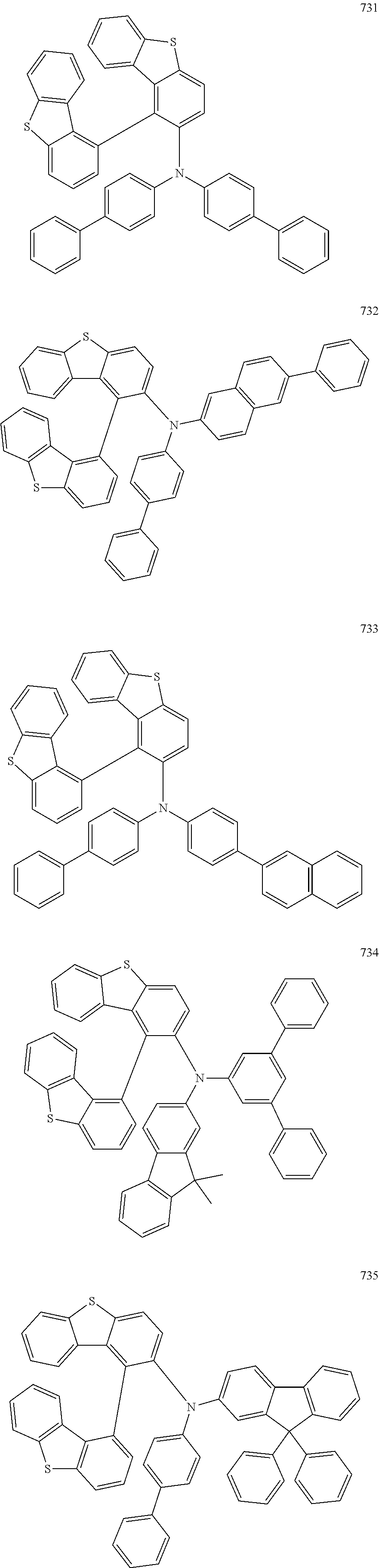 [SMILES: c1ccc(-c2ccc(N(c3ccc(-c4ccccc4)cc3)c3ccc4sc5ccccc5c4c3-c3cccc4sc5ccccc5c34)cc2)cc1 ; c1ccc(-c2ccc(N(c3ccc4cc(-c5ccccc5)ccc4c3)c3ccc4sc5ccccc5c4c3-c3cccc4sc5ccccc5c34)cc2)cc1 ; c1ccc(-c2ccc(N(c3ccc(-c4ccc5ccccc5c4)cc3)c3ccc4sc5ccccc5c4c3-c3cccc4sc5ccccc5c34)cc2)cc1 ; CC1(C)c2ccccc2-c2ccc(N(c3cc(-c4ccccc4)cc(-c4ccccc4)c3)c3ccc4sc5ccccc5c4c3-c3cccc4sc5ccccc5c34)cc21 ; c1ccc(-c2ccc(N(c3ccc4c(c3)C(c3ccccc3)(c3ccccc3)c3ccccc3-4)c3ccc4sc5ccccc5c4c3-c3cccc4sc5ccccc5c34)cc2)cc1]

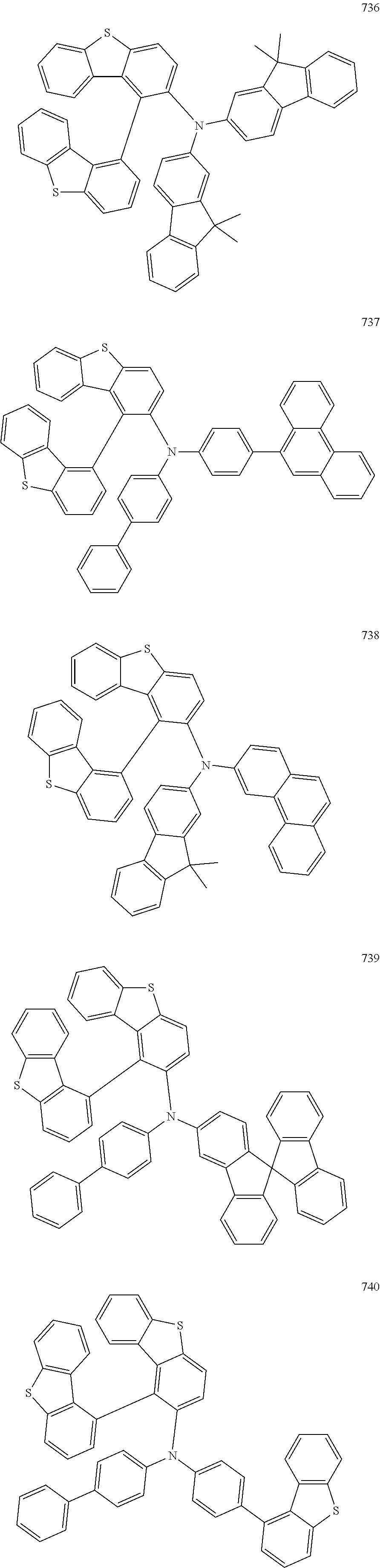 [SMILES: CC1(C)c2ccccc2-c2ccc(N(c3ccc4c(c3)C(C)(C)c3ccccc3-4)c3ccc4sc5ccccc5c4c3-c3cccc4sc5ccccc5c34)cc21 ; c1ccc(-c2ccc(N(c3ccc(-c4cc5ccccc5c5ccccc45)cc3)c3ccc4sc5ccccc5c4c3-c3cccc4sc5ccccc5c34)cc2)cc1 ; CC1(C)c2ccccc2-c2ccc(N(c3ccc4ccc5ccccc5c4c3)c3ccc4sc5ccccc5c4c3-c3cccc4sc5ccccc5c34)cc21 ; c1ccc(-c2ccc(N(c3ccc4c(c3)-c3ccccc3C43c4ccccc4-c4ccccc43)c3ccc4sc5ccccc5c4c3-c3cccc4sc5ccccc5c34)cc2)cc1 ; c1ccc(-c2ccc(N(c3ccc(-c4cccc5sc6ccccc6c45)cc3)c3ccc4sc5ccccc5c4c3-c3cccc4sc5ccccc5c34)cc2)cc1]

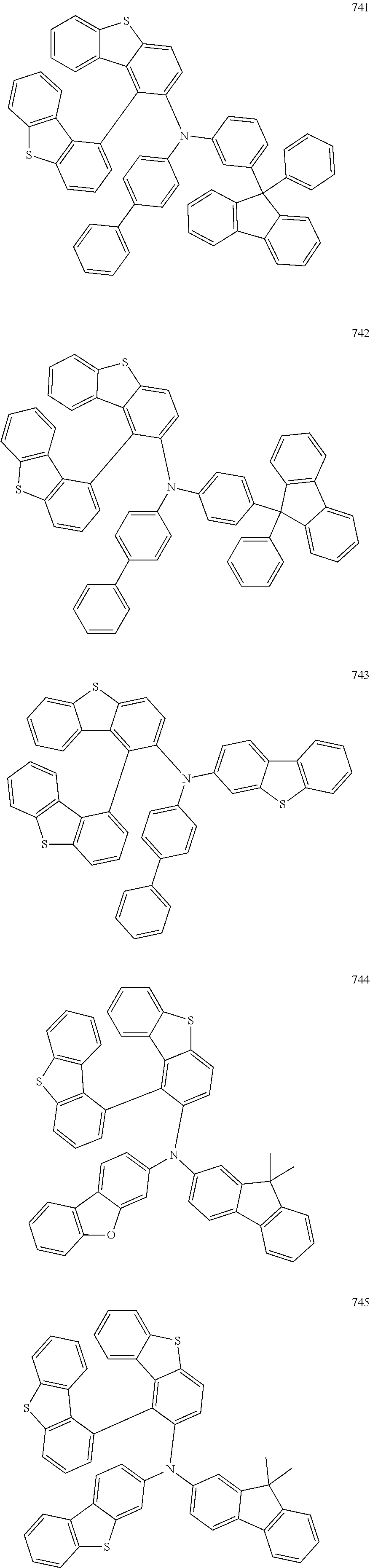 [SMILES: c1ccc(-c2ccc(N(c3cccc(C4(c5ccccc5)c5ccccc5-c5ccccc54)c3)c3ccc4sc5ccccc5c4c3-c3cccc4sc5ccccc5c34)cc2)cc1 ; c1ccc(-c2ccc(N(c3ccc(C4(c5ccccc5)c5ccccc5-c5ccccc54)cc3)c3ccc4sc5ccccc5c4c3-c3cccc4sc5ccccc5c34)cc2)cc1 ; c1ccc(-c2ccc(N(c3ccc4c(c3)sc3ccccc34)c3ccc4sc5ccccc5c4c3-c3cccc4sc5ccccc5c34)cc2)cc1 ; CC1(C)c2ccccc2-c2ccc(N(c3ccc4c(c3)oc3ccccc34)c3ccc4sc5ccccc5c4c3-c3cccc4sc5ccccc5c34)cc21 ; CC1(C)c2ccccc2-c2ccc(N(c3ccc4c(c3)sc3ccccc34)c3ccc4sc5ccccc5c4c3-c3cccc4sc5ccccc5c34)cc21]

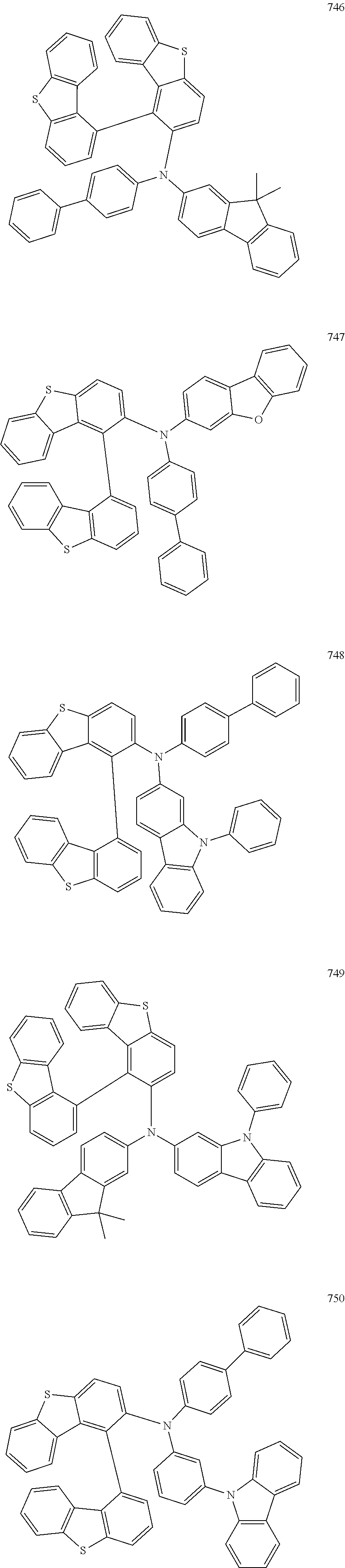 [SMILES: CC1(C)c2ccccc2-c2ccc(N(c3ccc(-c4ccccc4)cc3)c3ccc4sc5ccccc5c4c3-c3cccc4sc5ccccc5c34)cc21 ; c1ccc(-c2ccc(N(c3ccc4c(c3)oc3ccccc34)c3ccc4sc5ccccc5c4c3-c3cccc4sc5ccccc5c34)cc2)cc1 ; c1ccc(-c2ccc(N(c3ccc4c5ccccc5n(-c5ccccc5)c4c3)c3ccc4sc5ccccc5c4c3-c3cccc4sc5ccccc5c34)cc2)cc1 ; CC1(C)c2ccccc2-c2ccc(N(c3ccc4c5ccccc5n(-c5ccccc5)c4c3)c3ccc4sc5ccccc5c4c3-c3cccc4sc5ccccc5c34)cc21 ; c1ccc(-c2ccc(N(c3cccc(-n4c5ccccc5c5ccccc54)c3)c3ccc4sc5ccccc5c4c3-c3cccc4sc5ccccc5c34)cc2)cc1]

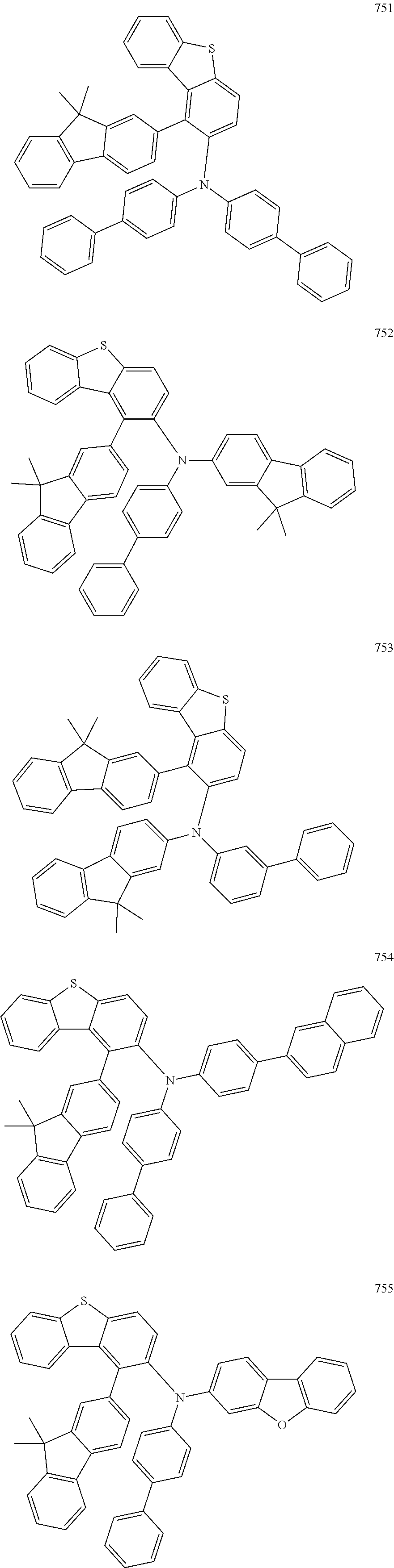 [SMILES: CC1(C)c2ccccc2-c2ccc(-c3c(N(c4ccc(-c5ccccc5)cc4)c4ccc(-c5ccccc5)cc4)ccc4sc5ccccc5c34)cc21 ; CC1(C)c2ccccc2-c2ccc(-c3c(N(c4ccc(-c5ccccc5)cc4)c4ccc5c(c4)C(C)(C)c4ccccc4-5)ccc4sc5ccccc5c34)cc21 ; CC1(C)c2ccccc2-c2ccc(-c3c(N(c4cccc(-c5ccccc5)c4)c4ccc5c(c4)C(C)(C)c4ccccc4-5)ccc4sc5ccccc5c34)cc21 ; CC1(C)c2ccccc2-c2ccc(-c3c(N(c4ccc(-c5ccccc5)cc4)c4ccc(-c5ccc6ccccc6c5)cc4)ccc4sc5ccccc5c34)cc21 ; CC1(C)c2ccccc2-c2ccc(-c3c(N(c4ccc(-c5ccccc5)cc4)c4ccc5c(c4)oc4ccccc45)ccc4sc5ccccc5c34)cc21]

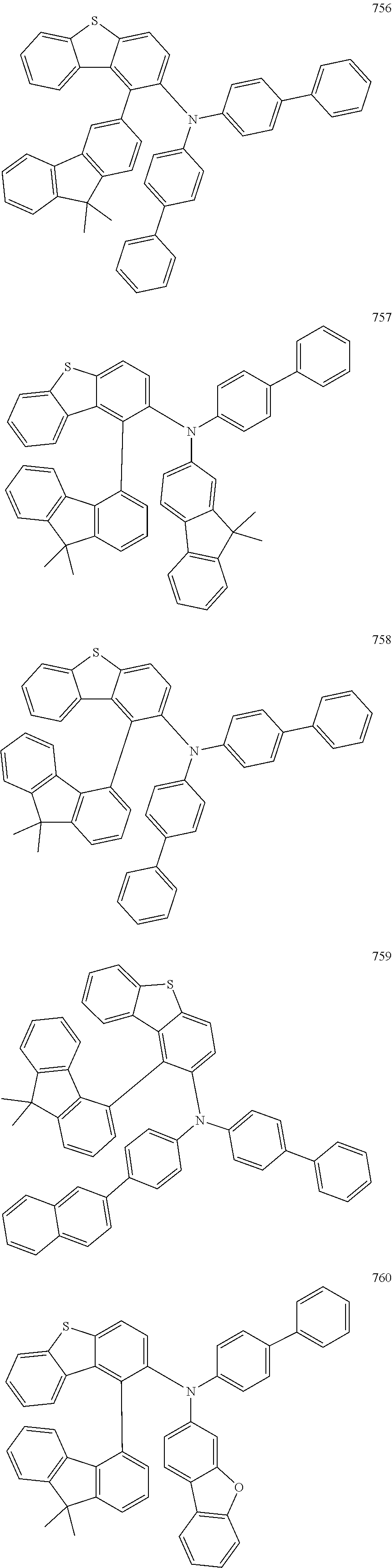 [SMILES: CC1(C)c2ccccc2-c2cc(-c3c(N(c4ccc(-c5ccccc5)cc4)c4ccc(-c5ccccc5)cc4)ccc4sc5ccccc5c34)ccc21 ; CC1(C)c2ccccc2-c2ccc(N(c3ccc(-c4ccccc4)cc3)c3ccc4sc5ccccc5c4c3-c3cccc4c3-c3ccccc3C4(C)C)cc21 ; CC1(C)c2ccccc2-c2c(-c3c(N(c4ccc(-c5ccccc5)cc4)c4ccc(-c5ccccc5)cc4)ccc4sc5ccccc5c34)cccc21 ; CC1(C)c2ccccc2-c2c(-c3c(N(c4ccc(-c5ccccc5)cc4)c4ccc(-c5ccc6ccccc6c5)cc4)ccc4sc5ccccc5c34)cccc21 ; CC1(C)c2ccccc2-c2c(-c3c(N(c4ccc(-c5ccccc5)cc4)c4ccc5c(c4)oc4ccccc45)ccc4sc5ccccc5c34)cccc21]

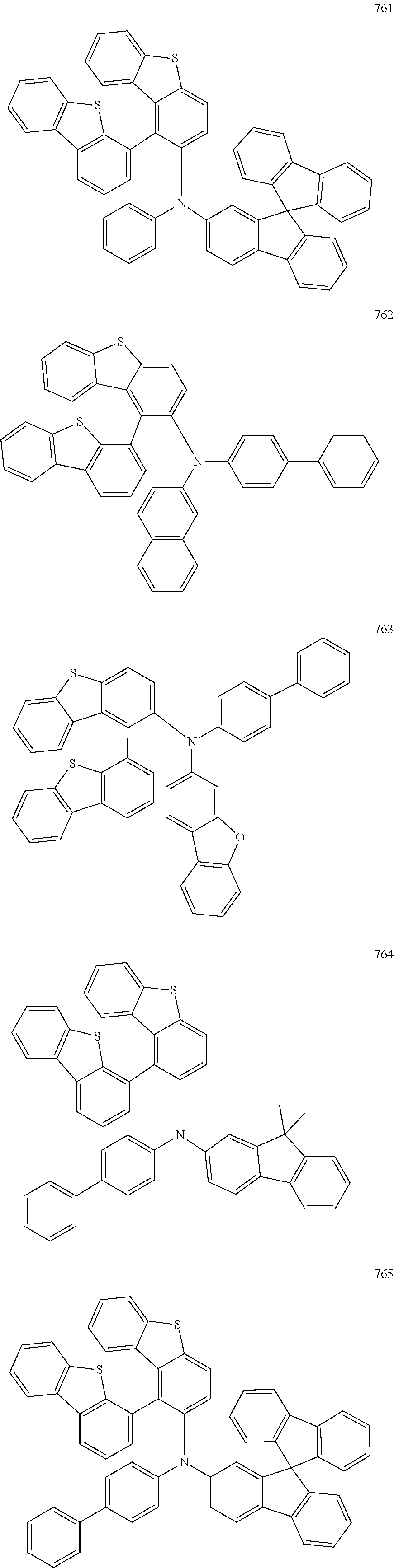 [SMILES: c1ccc(N(c2ccc3c(c2)C2(c4ccccc4-c4ccccc42)c2ccccc2-3)c2ccc3sc4ccccc4c3c2-c2cccc3c2sc2ccccc23)cc1 ; c1ccc(-c2ccc(N(c3ccc4ccccc4c3)c3ccc4sc5ccccc5c4c3-c3cccc4c3sc3ccccc34)cc2)cc1 ; c1ccc(-c2ccc(N(c3ccc4c(c3)oc3ccccc34)c3ccc4sc5ccccc5c4c3-c3cccc4c3sc3ccccc34)cc2)cc1 ; CC1(C)c2ccccc2-c2ccc(N(c3ccc(-c4ccccc4)cc3)c3ccc4sc5ccccc5c4c3-c3cccc4c3sc3ccccc34)cc21 ; c1ccc(-c2ccc(N(c3ccc4c(c3)C3(c5ccccc5-c5ccccc53)c3ccccc3-4)c3ccc4sc5ccccc5c4c3-c3cccc4c3sc3ccccc34)cc2)cc1]

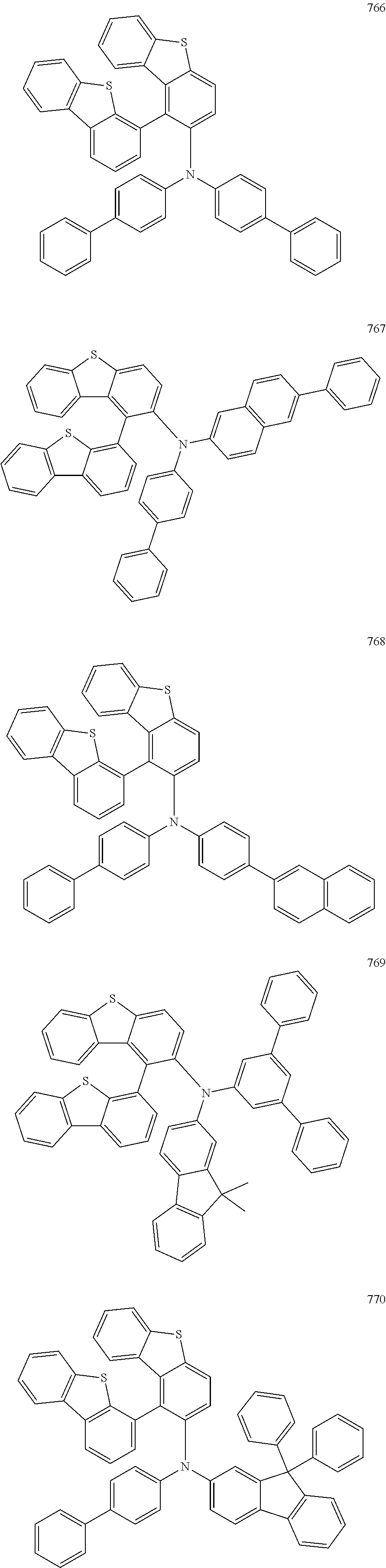 [SMILES: c1ccc(-c2ccc(N(c3ccc(-c4ccccc4)cc3)c3ccc4sc5ccccc5c4c3-c3cccc4c3sc3ccccc34)cc2)cc1 ; c1ccc(-c2ccc(N(c3ccc4cc(-c5ccccc5)ccc4c3)c3ccc4sc5ccccc5c4c3-c3cccc4c3sc3ccccc34)cc2)cc1 ; c1ccc(-c2ccc(N(c3ccc(-c4ccc5ccccc5c4)cc3)c3ccc4sc5ccccc5c4c3-c3cccc4c3sc3ccccc34)cc2)cc1 ; CC1(C)c2ccccc2-c2ccc(N(c3cc(-c4ccccc4)cc(-c4ccccc4)c3)c3ccc4sc5ccccc5c4c3-c3cccc4c3sc3ccccc34)cc21 ; c1ccc(-c2ccc(N(c3ccc4c(c3)C(c3ccccc3)(c3ccccc3)c3ccccc3-4)c3ccc4sc5ccccc5c4c3-c3cccc4c3sc3ccccc34)cc2)cc1]

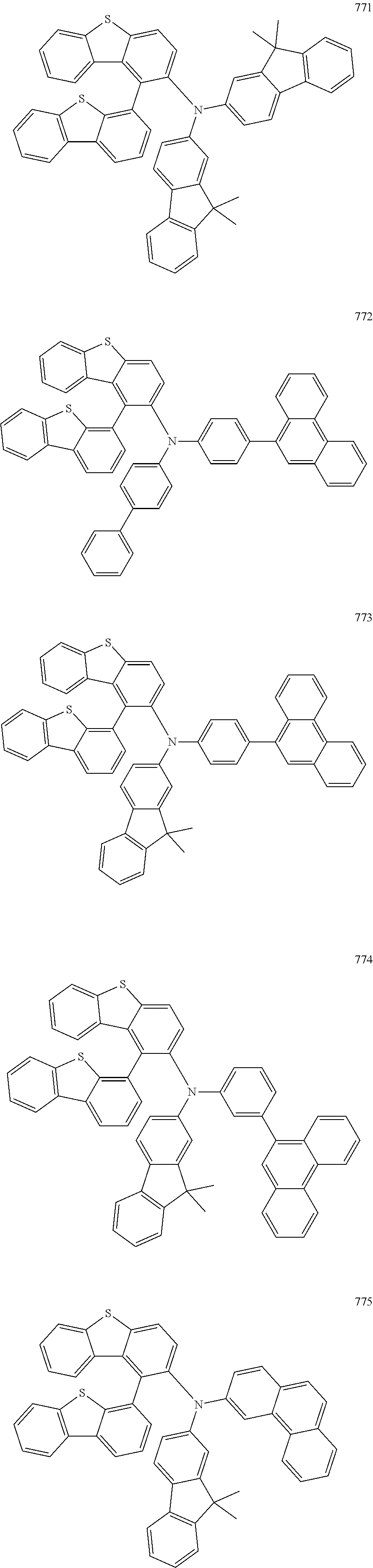 [SMILES: CC1(C)c2ccccc2-c2ccc(N(c3ccc4c(c3)C(C)(C)c3ccccc3-4)c3ccc4sc5ccccc5c4c3-c3cccc4c3sc3ccccc34)cc21 ; c1ccc(-c2ccc(N(c3ccc(-c4cc5ccccc5c5ccccc45)cc3)c3ccc4sc5ccccc5c4c3-c3cccc4c3sc3ccccc34)cc2)cc1 ; CC1(C)c2ccccc2-c2ccc(N(c3ccc(-c4cc5ccccc5c5ccccc45)cc3)c3ccc4sc5ccccc5c4c3-c3cccc4c3sc3ccccc34)cc21 ; CC1(C)c2ccccc2-c2ccc(N(c3cccc(-c4cc5ccccc5c5ccccc45)c3)c3ccc4sc5ccccc5c4c3-c3cccc4c3sc3ccccc34)cc21 ; CC1(C)c2ccccc2-c2ccc(N(c3ccc4ccc5ccccc5c4c3)c3ccc4sc5ccccc5c4c3-c3cccc4c3sc3ccccc34)cc21]

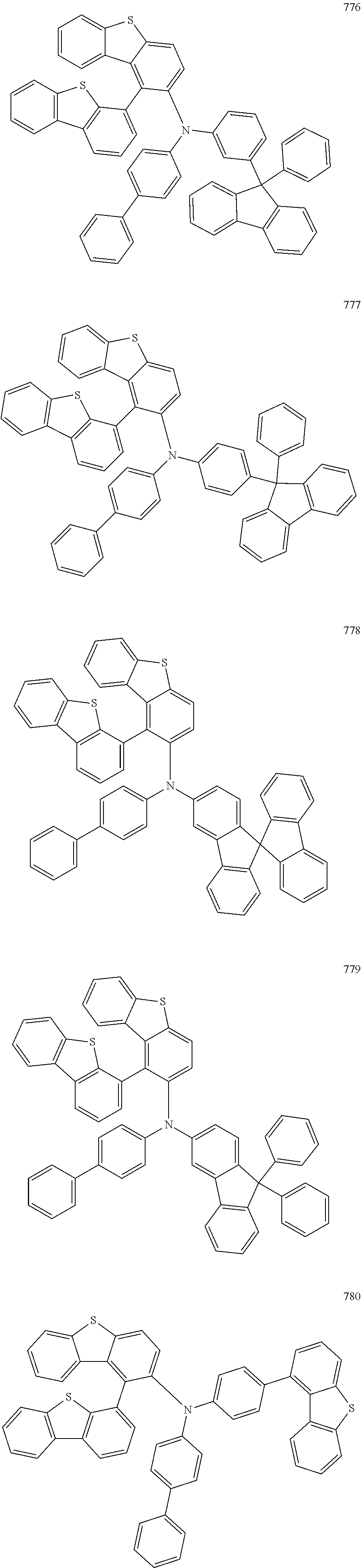

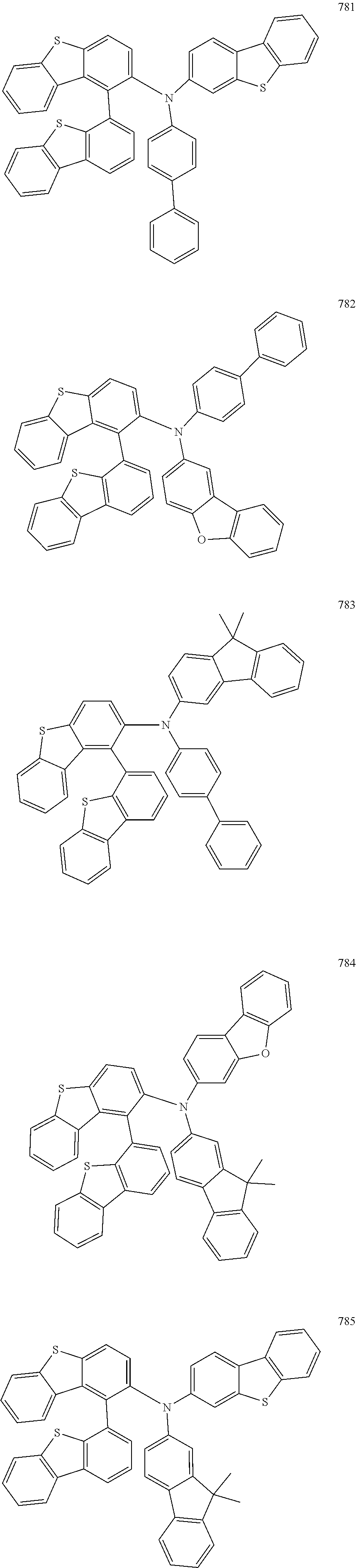 [SMILES: c1ccc(-c2ccc(N(c3ccc4c(c3)sc3ccccc34)c3ccc4sc5ccccc5c4c3-c3cccc4c3sc3ccccc34)cc2)cc1 ; c1ccc(-c2ccc(N(c3ccc4oc5ccccc5c4c3)c3ccc4sc5ccccc5c4c3-c3cccc4c3sc3ccccc34)cc2)cc1 ; CC1(C)c2ccccc2-c2cc(N(c3ccc(-c4ccccc4)cc3)c3ccc4sc5ccccc5c4c3-c3cccc4c3sc3ccccc34)ccc21 ; CC1(C)c2ccccc2-c2ccc(N(c3ccc4c(c3)oc3ccccc34)c3ccc4sc5ccccc5c4c3-c3cccc4c3sc3ccccc34)cc21 ; CC1(C)c2ccccc2-c2ccc(N(c3ccc4c(c3)sc3ccccc34)c3ccc4sc5ccccc5c4c3-c3cccc4c3sc3ccccc34)cc21]

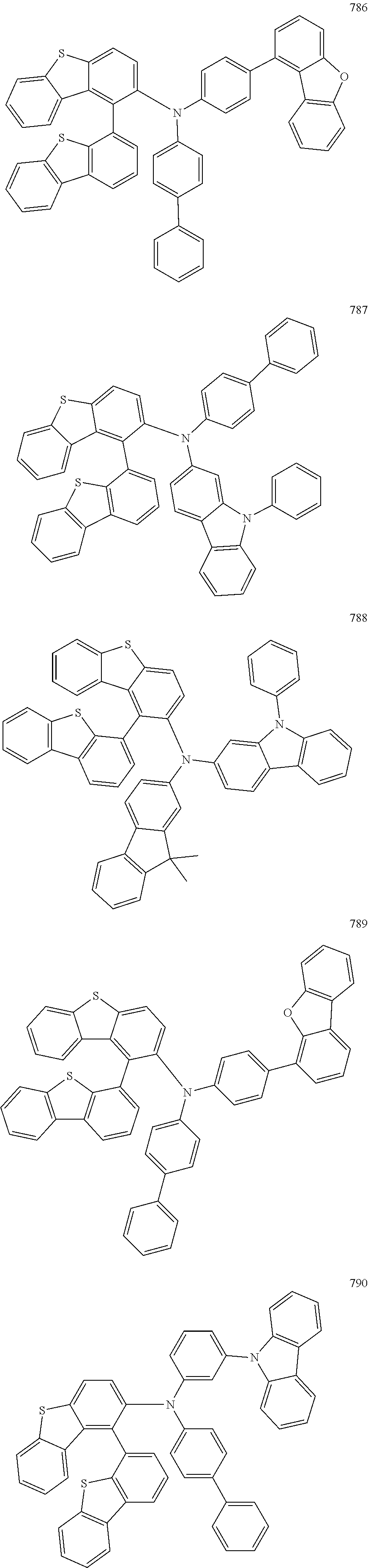 [SMILES: c1ccc(-c2ccc(N(c3ccc(-c4cccc5oc6ccccc6c45)cc3)c3ccc4sc5ccccc5c4c3-c3cccc4c3sc3ccccc34)cc2)cc1 ; c1ccc(-c2ccc(N(c3ccc4c5ccccc5n(-c5ccccc5)c4c3)c3ccc4sc5ccccc5c4c3-c3cccc4c3sc3ccccc34)cc2)cc1 ; CC1(C)c2ccccc2-c2ccc(N(c3ccc4c5ccccc5n(-c5ccccc5)c4c3)c3ccc4sc5ccccc5c4c3-c3cccc4c3sc3ccccc34)cc21 ; c1ccc(-c2ccc(N(c3ccc(-c4cccc5c4oc4ccccc45)cc3)c3ccc4sc5ccccc5c4c3-c3cccc4c3sc3ccccc34)cc2)cc1 ; c1ccc(-c2ccc(N(c3cccc(-n4c5ccccc5c5ccccc54)c3)c3ccc4sc5ccccc5c4c3-c3cccc4c3sc3ccccc34)cc2)cc1]

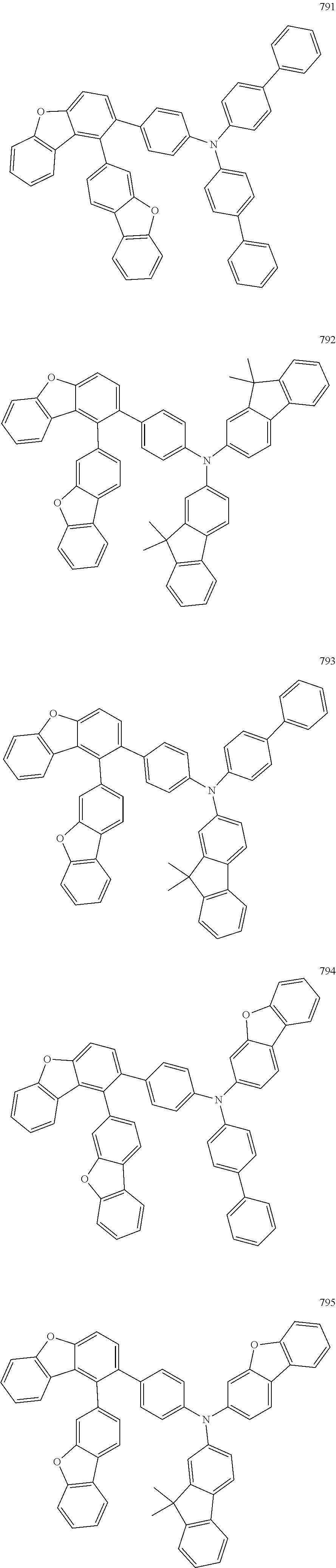

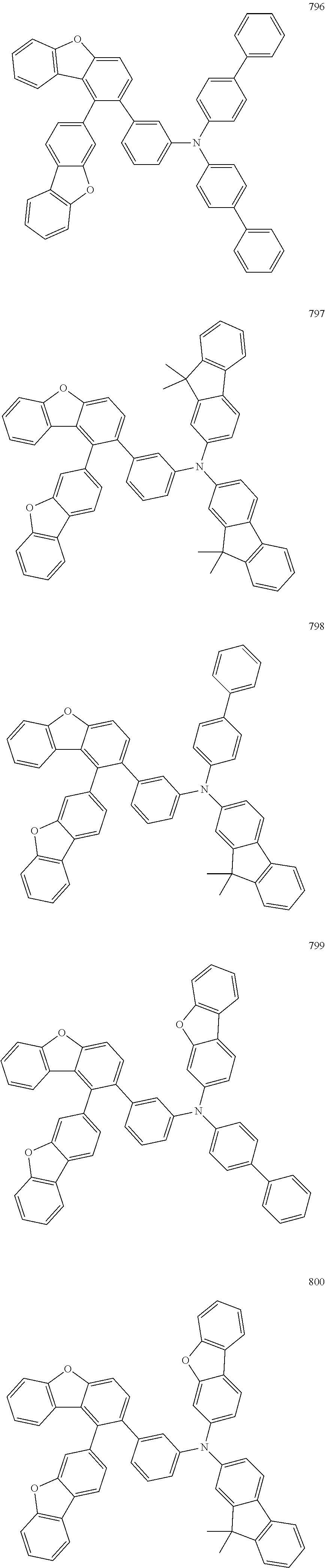

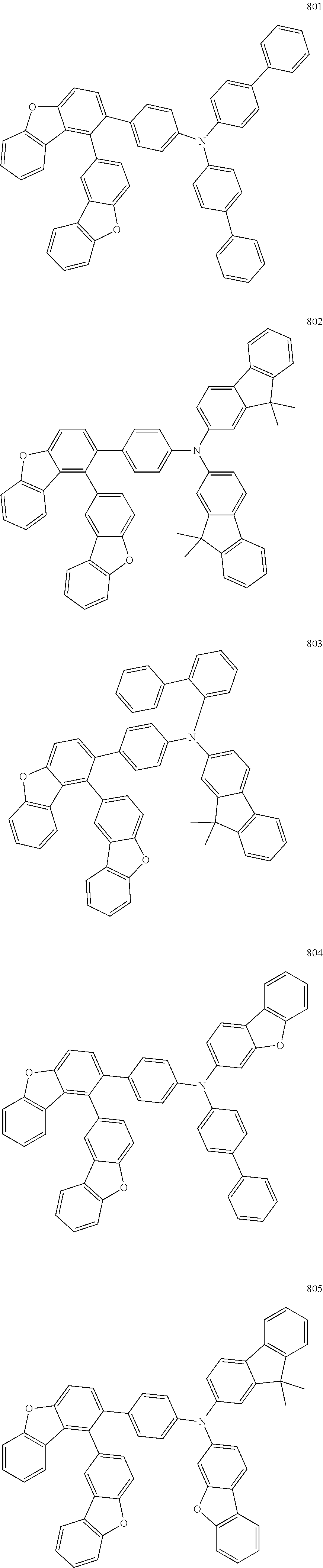

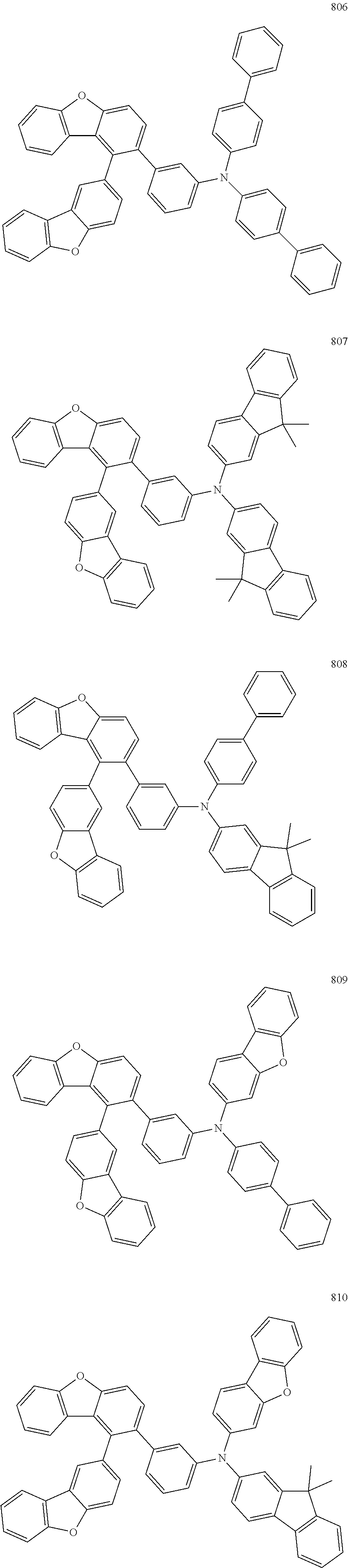

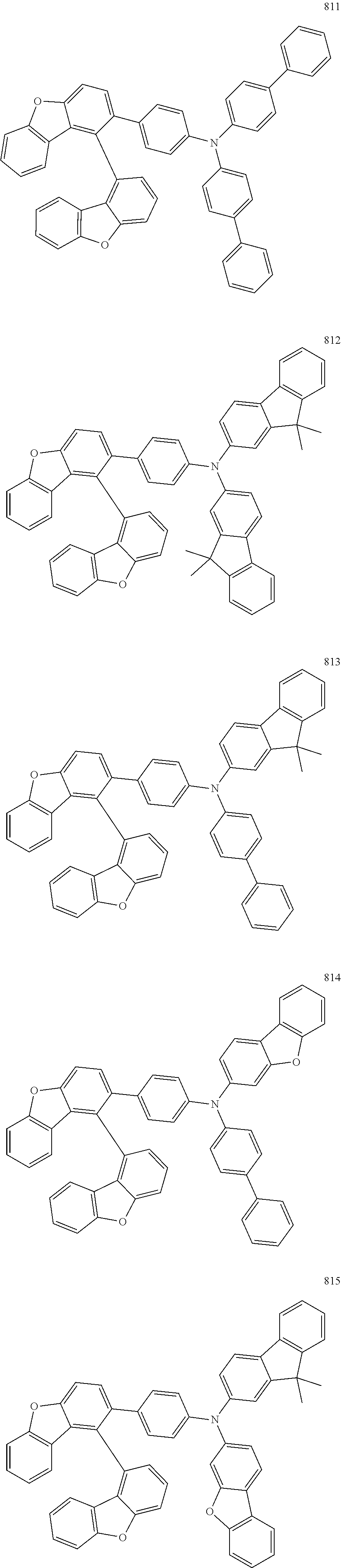

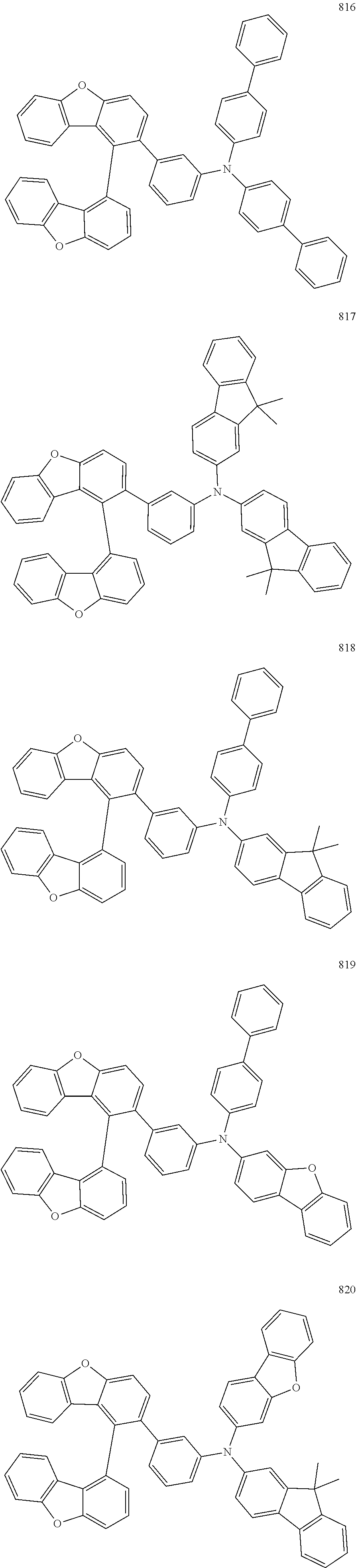

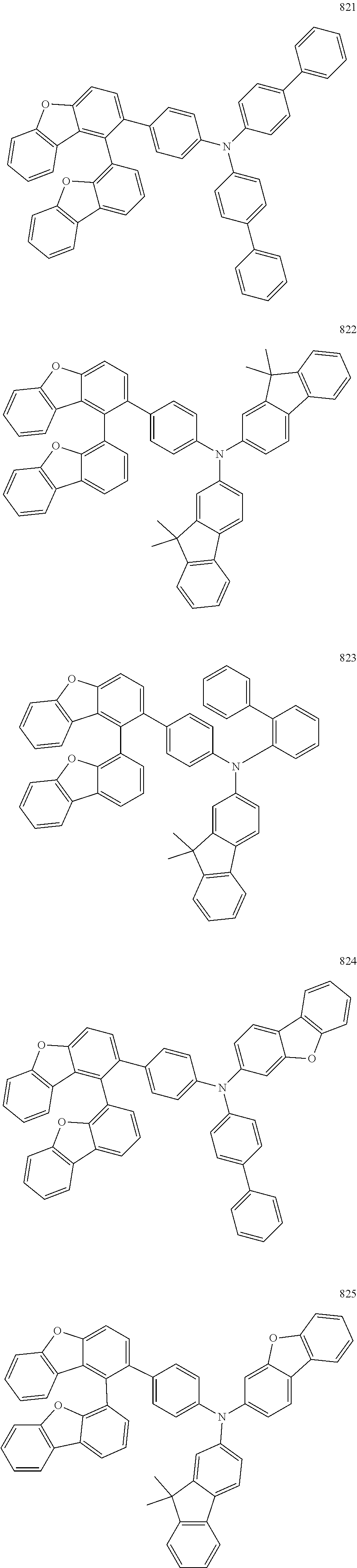

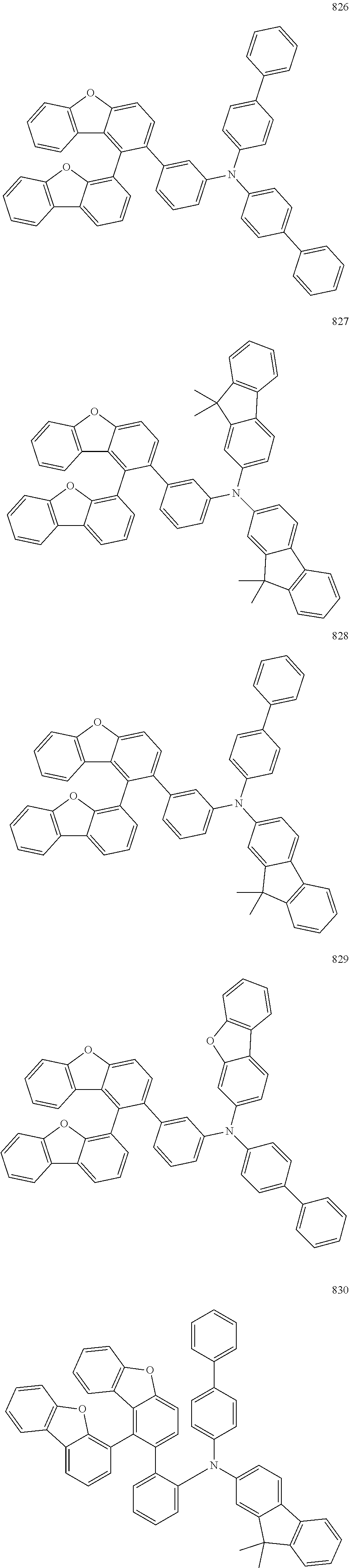

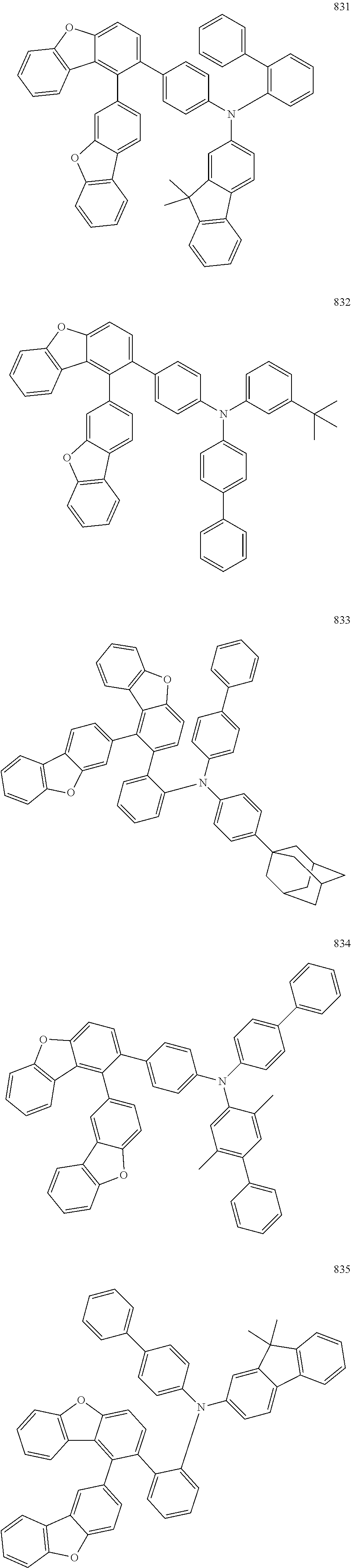 [SMILES: CC1(C)c2ccccc2-c2ccc(N(c3ccc(-c4ccc5oc6ccccc6c5c4-c4ccc5c(c4)oc4ccccc45)cc3)c3ccccc3-c3ccccc3)cc21 ; CC(C)(C)c1cccc(N(c2ccc(-c3ccccc3)cc2)c2ccc(-c3ccc4oc5ccccc5c4c3-c3ccc4c(c3)oc3ccccc34)cc2)c1 ; c1ccc(-c2ccc(N(c3ccc(C45CC6CC(CC(C6)C4)C5)cc3)c3ccccc3-c3ccc4oc5ccccc5c4c3-c3ccc4c(c3)oc3ccccc34)cc2)cc1 ; Cc1cc(N(c2ccc(-c3ccccc3)cc2)c2ccc(-c3ccc4oc5ccccc5c4c3-c3ccc4oc5ccccc5c4c3)cc2)c(C)cc1-c1ccccc1 ; CC1(C)c2ccccc2-c2ccc(N(c3ccc(-c4ccccc4)cc3)c3ccccc3-c3ccc4oc5ccccc5c4c3-c3ccc4oc5ccccc5c4c3)cc21]

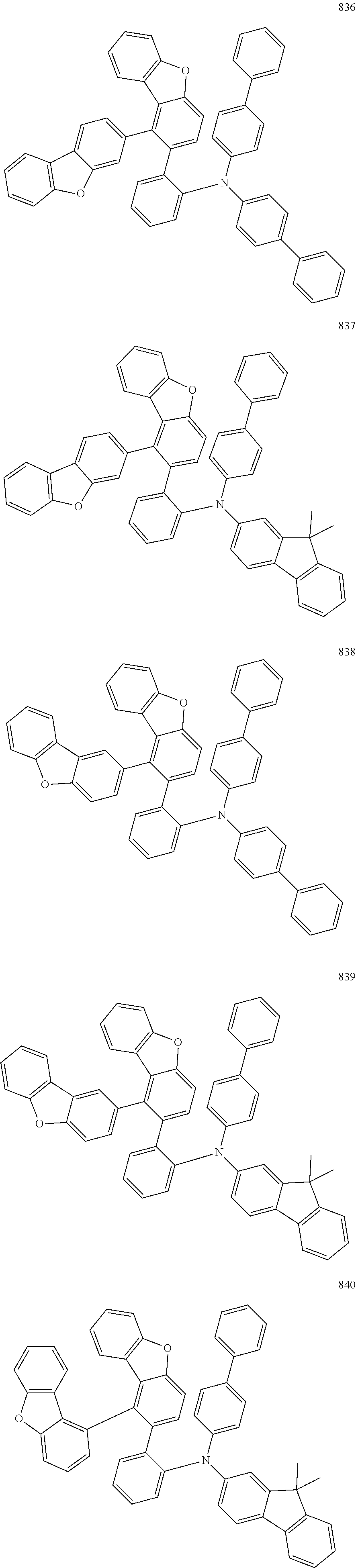 [SMILES: c1ccc(-c2ccc(N(c3ccc(-c4ccccc4)cc3)c3ccccc3-c3ccc4oc5ccccc5c4c3-c3ccc4c(c3)oc3ccccc34)cc2)cc1 ; CC1(C)c2ccccc2-c2ccc(N(c3ccc(-c4ccccc4)cc3)c3ccccc3-c3ccc4oc5ccccc5c4c3-c3ccc4c(c3)oc3ccccc34)cc21 ; c1ccc(-c2ccc(N(c3ccc(-c4ccccc4)cc3)c3ccccc3-c3ccc4oc5ccccc5c4c3-c3ccc4oc5ccccc5c4c3)cc2)cc1 ; CC1(C)c2ccccc2-c2ccc(N(c3ccc(-c4ccccc4)cc3)c3ccccc3-c3ccc4oc5ccccc5c4c3-c3ccc4oc5ccccc5c4c3)cc21 ; CC1(C)c2ccccc2-c2ccc(N(c3ccc(-c4ccccc4)cc3)c3ccccc3-c3ccc4oc5ccccc5c4c3-c3cccc4oc5ccccc5c34)cc21]

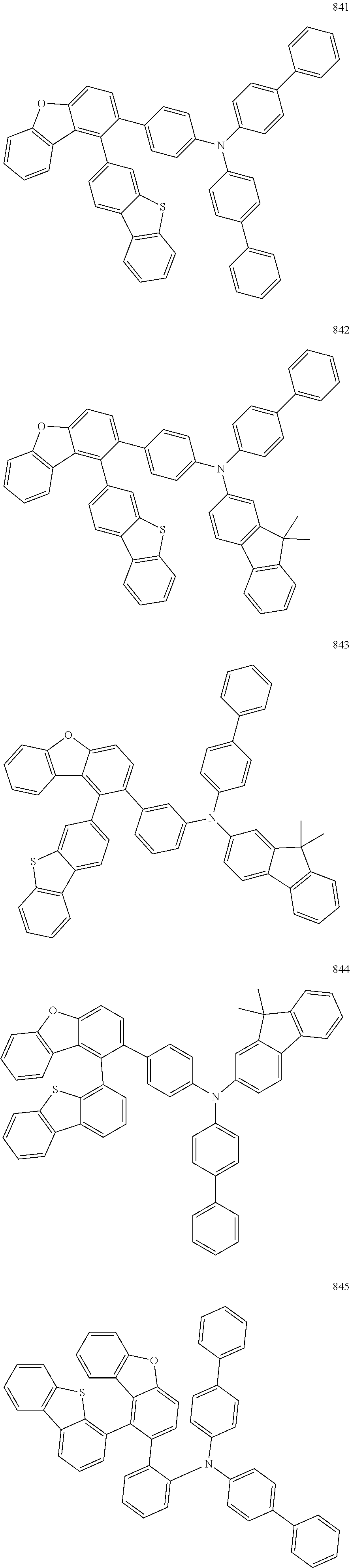 [SMILES: c1ccc(-c2ccc(N(c3ccc(-c4ccccc4)cc3)c3ccc(-c4ccc5oc6ccccc6c5c4-c4ccc5c(c4)sc4ccccc45)cc3)cc2)cc1 ; CC1(C)c2ccccc2-c2ccc(N(c3ccc(-c4ccccc4)cc3)c3ccc(-c4ccc5oc6ccccc6c5c4-c4ccc5c(c4)sc4ccccc45)cc3)cc21 ; CC1(C)c2ccccc2-c2ccc(N(c3ccc(-c4ccccc4)cc3)c3cccc(-c4ccc5oc6ccccc6c5c4-c4ccc5c(c4)sc4ccccc45)c3)cc21 ; CC1(C)c2ccccc2-c2ccc(N(c3ccc(-c4ccccc4)cc3)c3ccc(-c4ccc5oc6ccccc6c5c4-c4cccc5c4sc4ccccc45)cc3)cc21 ; c1ccc(-c2ccc(N(c3ccc(-c4ccccc4)cc3)c3ccccc3-c3ccc4oc5ccccc5c4c3-c3cccc4c3sc3ccccc34)cc2)cc1]

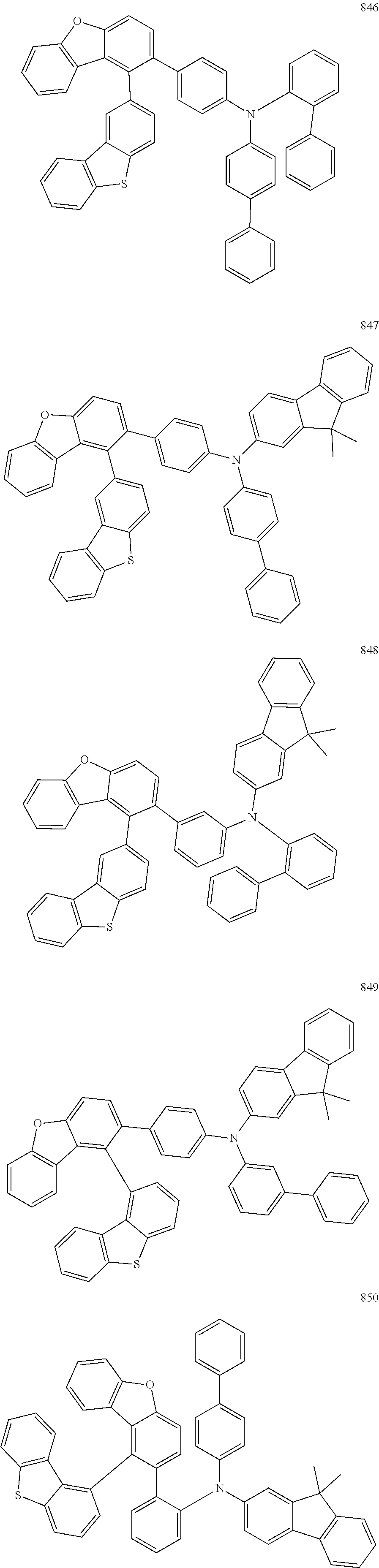

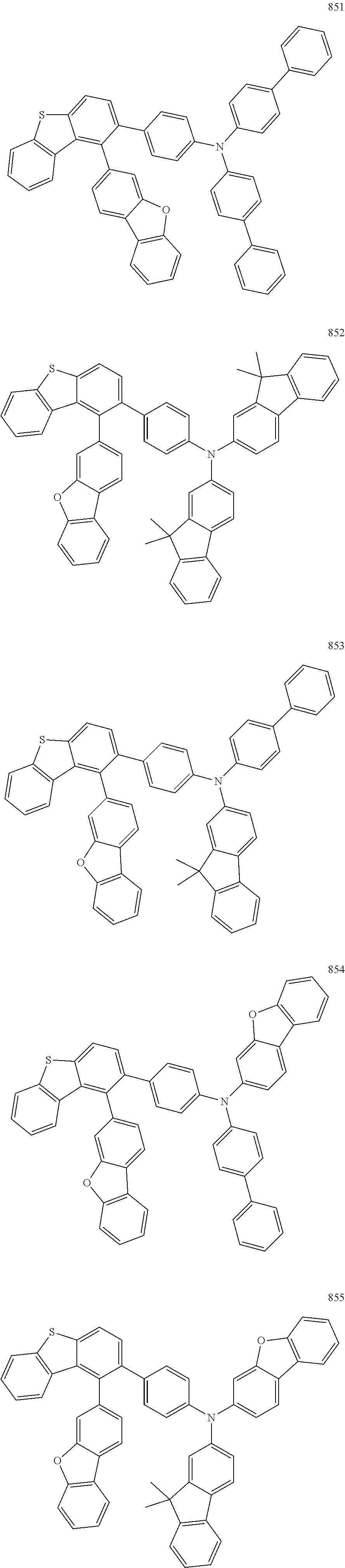

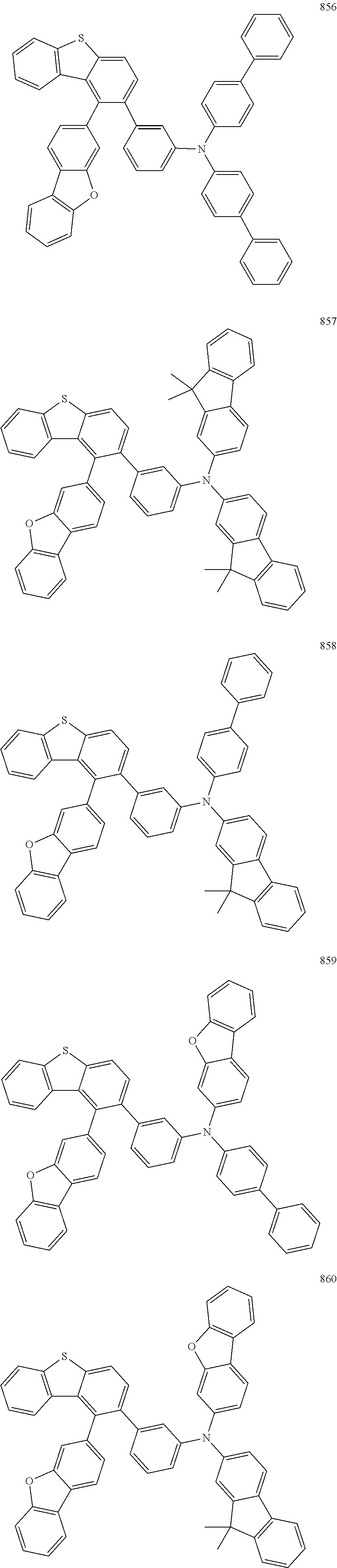

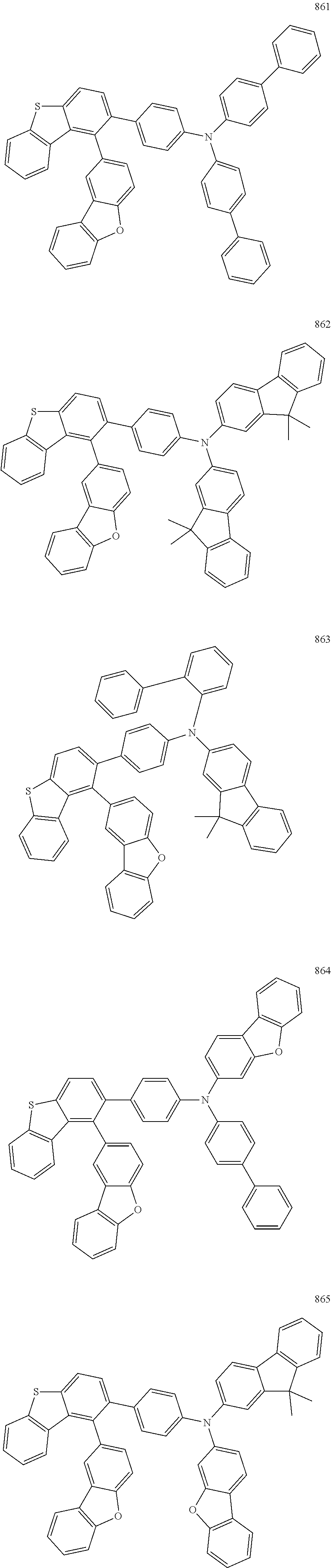

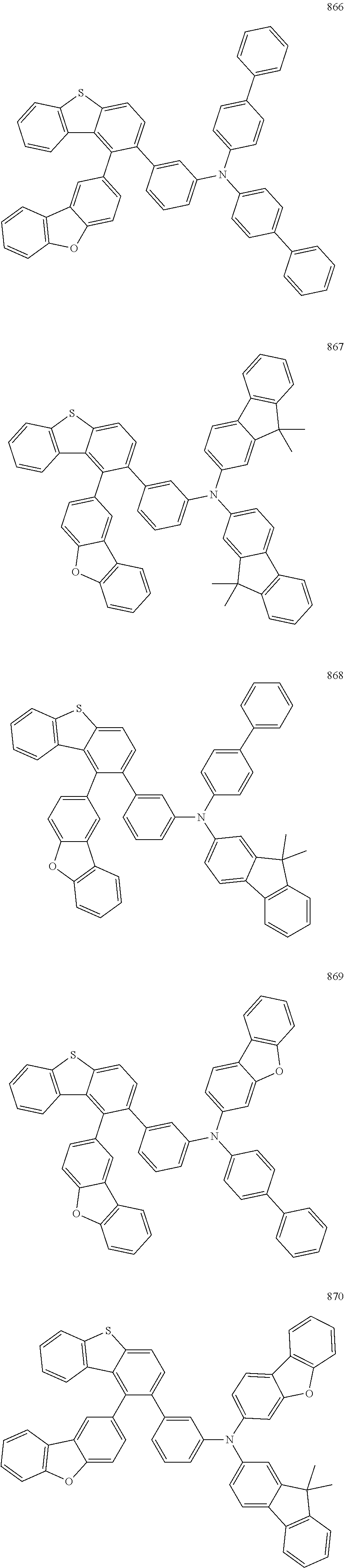

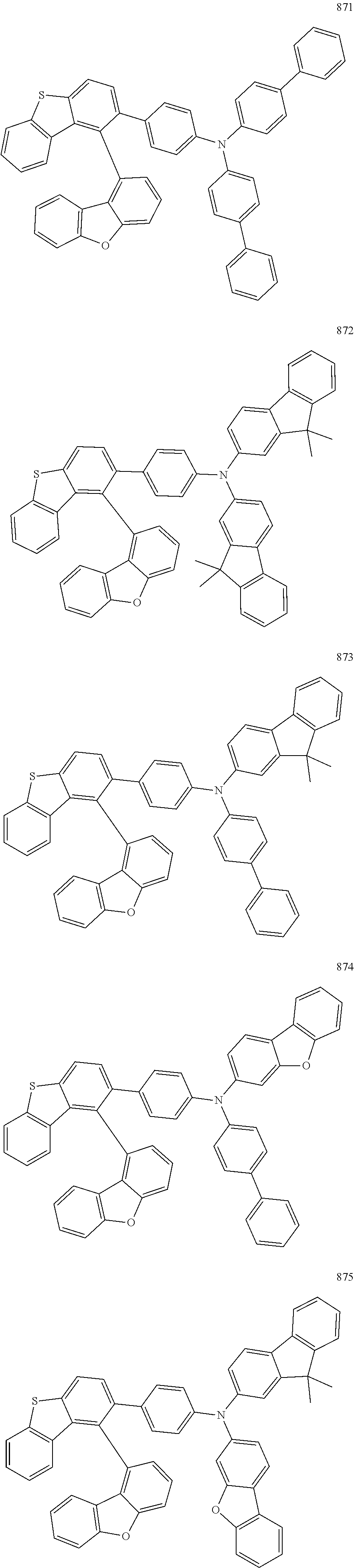

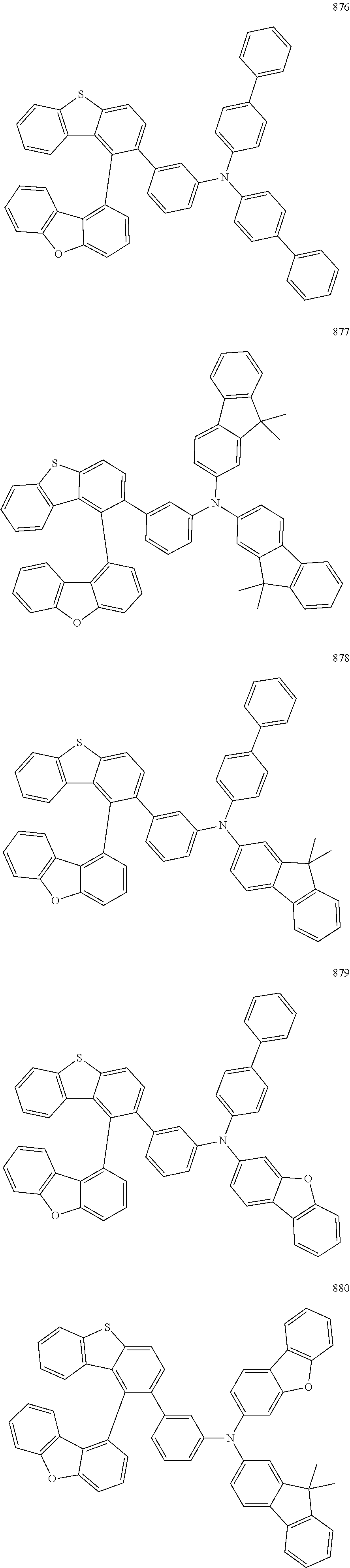

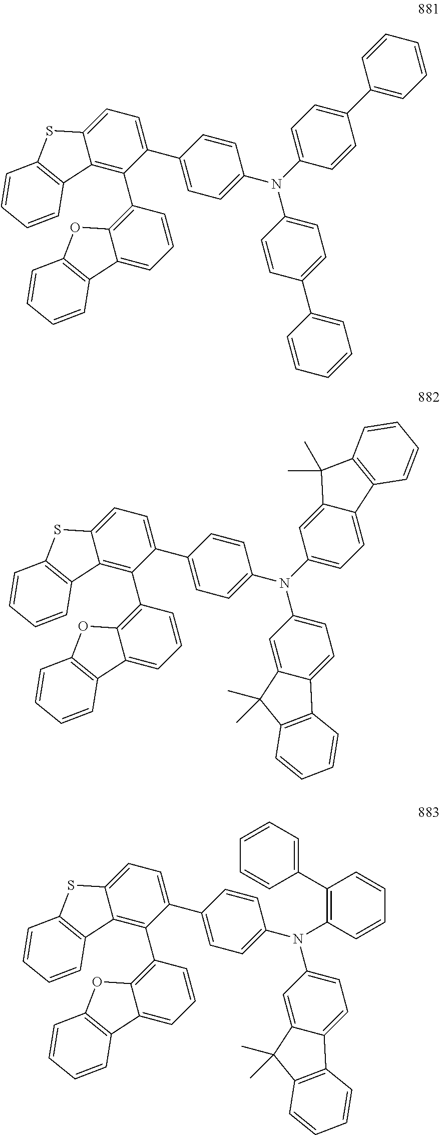 [SMILES: c1ccc(-c2ccc(N(c3ccc(-c4ccccc4)cc3)c3ccc(-c4ccc5sc6ccccc6c5c4-c4cccc5c4oc4ccccc45)cc3)cc2)cc1 ; CC1(C)c2ccccc2-c2ccc(N(c3ccc(-c4ccc5sc6ccccc6c5c4-c4cccc5c4oc4ccccc45)cc3)c3ccc4c(c3)C(C)(C)c3ccccc3-4)cc21 ; CC1(C)c2ccccc2-c2ccc(N(c3ccc(-c4ccc5sc6ccccc6c5c4-c4cccc5c4oc4ccccc45)cc3)c3ccccc3-c3ccccc3)cc21]

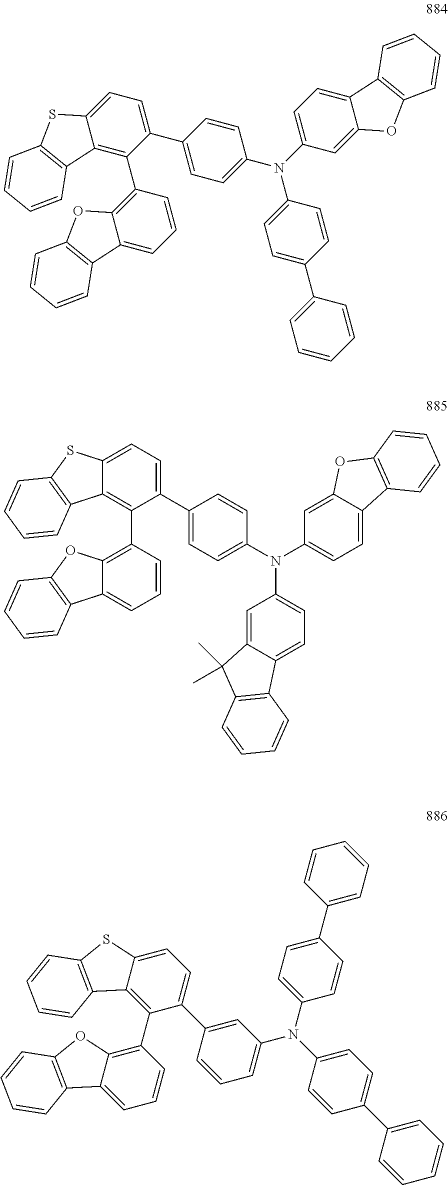 [SMILES: c1ccc(-c2ccc(N(c3ccc(-c4ccc5sc6ccccc6c5c4-c4cccc5c4oc4ccccc45)cc3)c3ccc4c(c3)oc3ccccc34)cc2)cc1 ; CC1(C)c2ccccc2-c2ccc(N(c3ccc(-c4ccc5sc6ccccc6c5c4-c4cccc5c4oc4ccccc45)cc3)c3ccc4c(c3)oc3ccccc34)cc21 ; c1ccc(-c2ccc(N(c3ccc(-c4ccccc4)cc3)c3cccc(-c4ccc5sc6ccccc6c5c4-c4cccc5c4oc4ccccc45)c3)cc2)cc1]

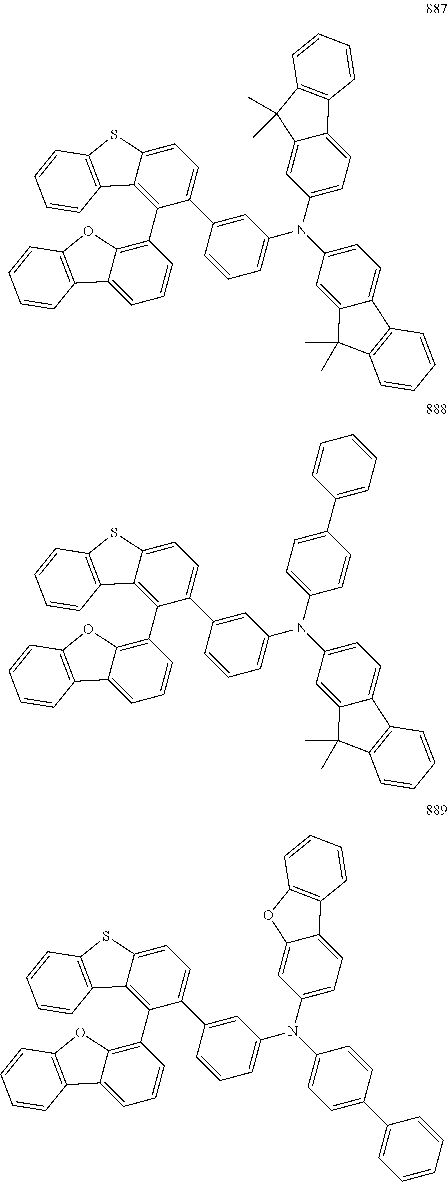 [SMILES: CC1(C)c2ccccc2-c2ccc(N(c3cccc(-c4ccc5sc6ccccc6c5c4-c4cccc5c4oc4ccccc45)c3)c3ccc4c(c3)C(C)(C)c3ccccc3-4)cc21 ; CC1(C)c2ccccc2-c2ccc(N(c3ccc(-c4ccccc4)cc3)c3cccc(-c4ccc5sc6ccccc6c5c4-c4cccc5c4oc4ccccc45)c3)cc21 ; c1ccc(-c2ccc(N(c3cccc(-c4ccc5sc6ccccc6c5c4-c4cccc5c4oc4ccccc45)c3)c3ccc4c(c3)oc3ccccc34)cc2)cc1]

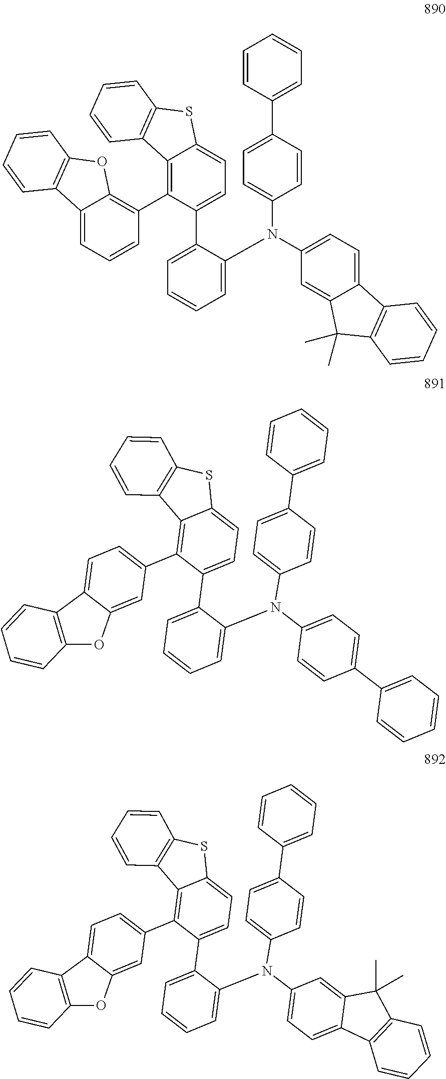 [SMILES: CC1(C)c2ccccc2-c2ccc(N(c3ccc(-c4ccccc4)cc3)c3ccccc3-c3ccc4sc5ccccc5c4c3-c3cccc4c3oc3ccccc34)cc21 ; c1ccc(-c2ccc(N(c3ccc(-c4ccccc4)cc3)c3ccccc3-c3ccc4sc5ccccc5c4c3-c3ccc4c(c3)oc3ccccc34)cc2)cc1 ; CC1(C)c2ccccc2-c2ccc(N(c3ccc(-c4ccccc4)cc3)c3ccccc3-c3ccc4sc5ccccc5c4c3-c3ccc4c(c3)oc3ccccc34)cc21]

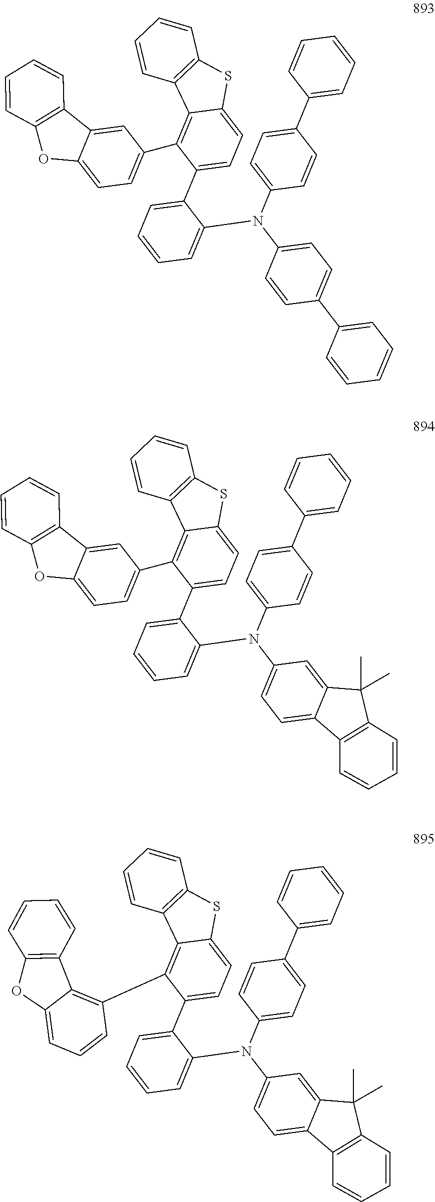 [SMILES: c1ccc(-c2ccc(N(c3ccc(-c4ccccc4)cc3)c3ccccc3-c3ccc4sc5ccccc5c4c3-c3ccc4oc5ccccc5c4c3)cc2)cc1 ; CC1(C)c2ccccc2-c2ccc(N(c3ccc(-c4ccccc4)cc3)c3ccccc3-c3ccc4sc5ccccc5c4c3-c3ccc4oc5ccccc5c4c3)cc21 ; CC1(C)c2ccccc2-c2ccc(N(c3ccc(-c4ccccc4)cc3)c3ccccc3-c3ccc4sc5ccccc5c4c3-c3cccc4oc5ccccc5c34)cc21]

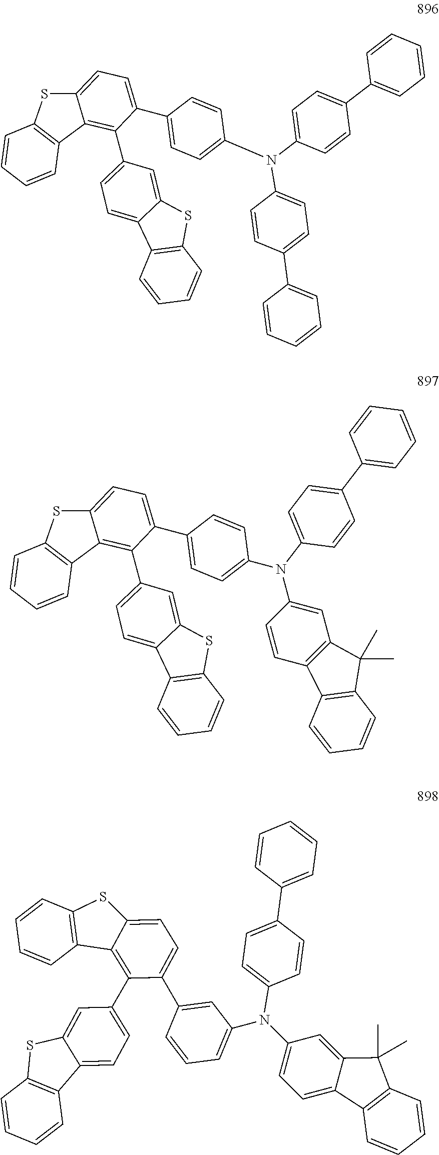 [SMILES: c1ccc(-c2ccc(N(c3ccc(-c4ccccc4)cc3)c3ccc(-c4ccc5sc6ccccc6c5c4-c4ccc5c(c4)sc4ccccc45)cc3)cc2)cc1 ; CC1(C)c2ccccc2-c2ccc(N(c3ccc(-c4ccccc4)cc3)c3ccc(-c4ccc5sc6ccccc6c5c4-c4ccc5c(c4)sc4ccccc45)cc3)cc21 ; CC1(C)c2ccccc2-c2ccc(N(c3ccc(-c4ccccc4)cc3)c3cccc(-c4ccc5sc6ccccc6c5c4-c4ccc5c(c4)sc4ccccc45)c3)cc21]

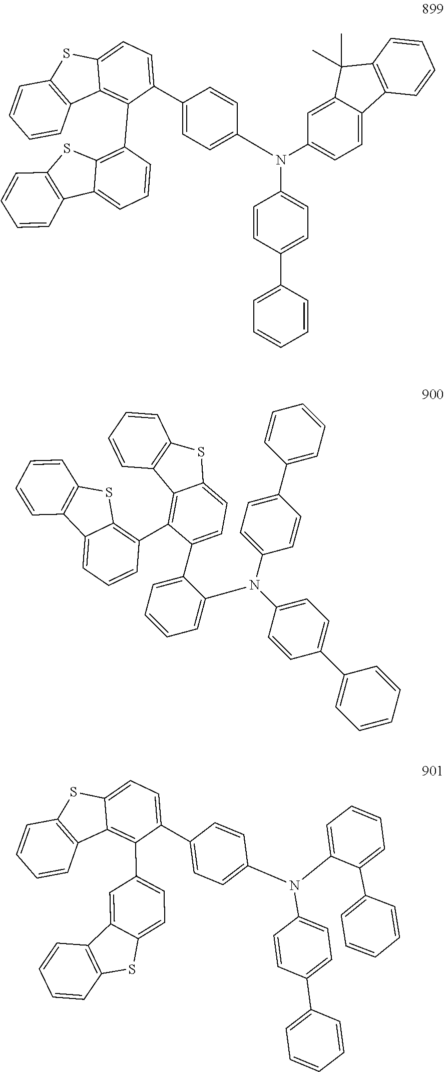 [SMILES: CC1(C)c2ccccc2-c2ccc(N(c3ccc(-c4ccccc4)cc3)c3ccc(-c4ccc5sc6ccccc6c5c4-c4cccc5c4sc4ccccc45)cc3)cc21 ; c1ccc(-c2ccc(N(c3ccc(-c4ccccc4)cc3)c3ccccc3-c3ccc4sc5ccccc5c4c3-c3cccc4c3sc3ccccc34)cc2)cc1 ; c1ccc(-c2ccc(N(c3ccc(-c4ccc5sc6ccccc6c5c4-c4ccc5sc6ccccc6c5c4)cc3)c3ccccc3-c3ccccc3)cc2)cc1]

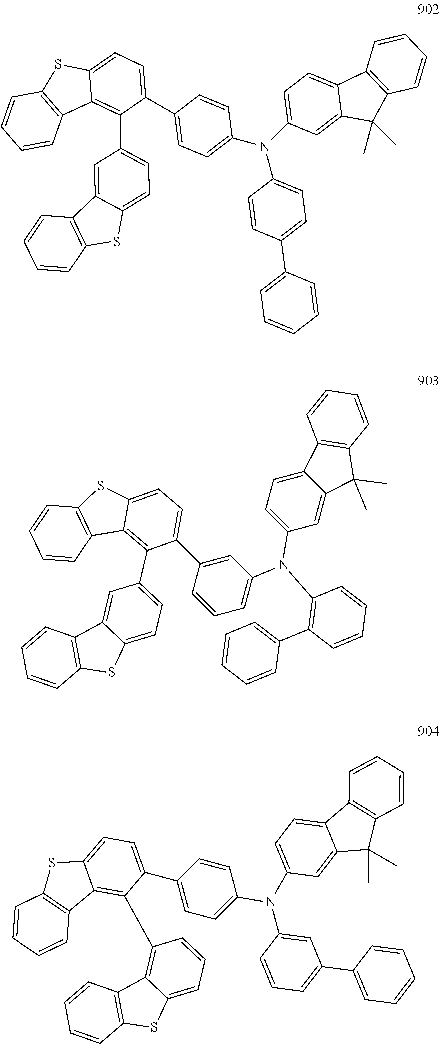 [SMILES: CC1(C)c2ccccc2-c2ccc(N(c3ccc(-c4ccccc4)cc3)c3ccc(-c4ccc5sc6ccccc6c5c4-c4ccc5sc6ccccc6c5c4)cc3)cc21 ; CC1(C)c2ccccc2-c2ccc(N(c3cccc(-c4ccc5sc6ccccc6c5c4-c4ccc5sc6ccccc6c5c4)c3)c3ccccc3-c3ccccc3)cc21 ; CC1(C)c2ccccc2-c2ccc(N(c3ccc(-c4ccc5sc6ccccc6c5c4-c4cccc5sc6ccccc6c45)cc3)c3cccc(-c4ccccc4)c3)cc21]

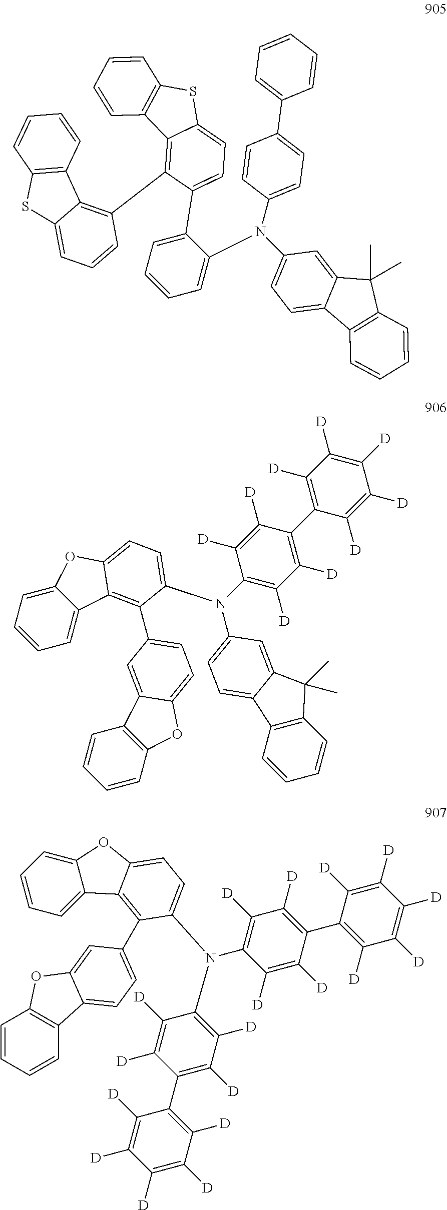 [SMILES: CC1(C)c2ccccc2-c2ccc(N(c3ccc(-c4ccccc4)cc3)c3ccccc3-c3ccc4sc5ccccc5c4c3-c3cccc4sc5ccccc5c34)cc21 ; [2H]c1c([2H])c([2H])c(-c2c([2H])c([2H])c(N(c3ccc4c(c3)C(C)(C)c3ccccc3-4)c3ccc4oc5ccccc5c4c3-c3ccc4oc5ccccc5c4c3)c([2H])c2[2H])c([2H])c1[2H] ; [2H]c1c([2H])c([2H])c(-c2c([2H])c([2H])c(N(c3ccc4oc5ccccc5c4c3-c3ccc4c(c3)oc3ccccc34)c3c([2H])c([2H])c(-c4c([2H])c([2H])c([2H])c([2H])c4[2H])c([2H])c3[2H])c([2H])c2[2H])c([2H])c1[2H]]

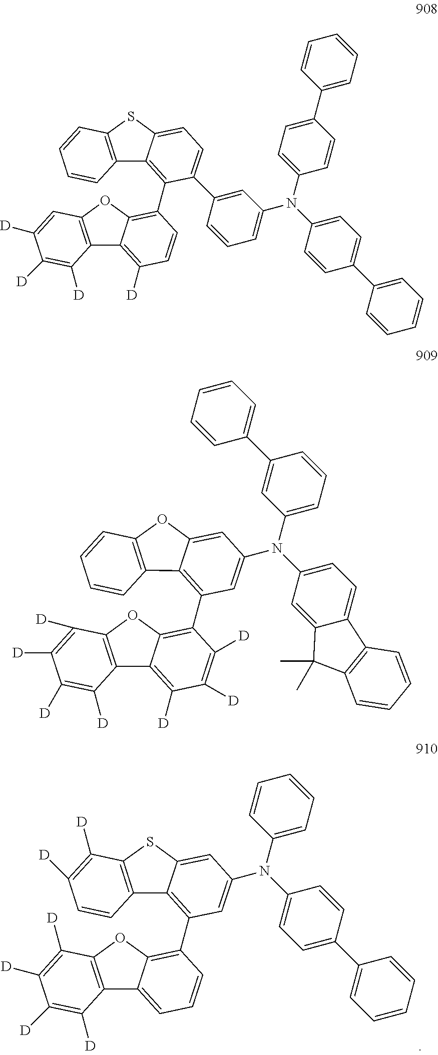 [SMILES: [2H]c1cc2oc3c(-c4c(-c5cccc(N(c6ccc(-c7ccccc7)cc6)c6ccc(-c7ccccc7)cc6)c5)ccc5sc6ccccc6c45)ccc([2H])c3c2c([2H])c1[2H] ; [2H]c1c([2H])c([2H])c2c(oc3c(-c4cc(N(c5cccc(-c6ccccc6)c5)c5ccc6c(c5)C(C)(C)c5ccccc5-6)cc5oc6ccccc6c45)c([2H])c([2H])c([2H])c32)c1[2H] ; [2H]c1ccc2c(sc3cc(N(c4ccccc4)c4ccc(-c5ccccc5)cc4)cc(-c4cccc5c4oc4c([2H])c([2H])c([2H])c([2H])c45)c32)c1[2H]]

11. An organic electroluminescent device, comprising an anode and a cathode disposed opposite to each other, and a functional layer disposed between the anode and the cathode, characterized in that the functional layer comprises the arylamine compound of claim 1.

12. The organic delectroluminescent device according to claim 11, wherein the functional layer comprises a hole auxiliary layer, and the hole auxiliary layer comprises the arylamine compound.

13. An electronic apparatus, comprising the organic electroluminescent device of claim 11.
